(19) United States
(12) Patent Application Publication
(10) Pub. No.: US 2025/0189534 A1
(43) Pub. Date: Jun. 12, 2025

(54) SAMPLE PREPARATION FOR GLYCOPROTEOMIC ANALYSIS THAT INCLUDES DIAGNOSIS OF DISEASE

(71) Applicant: VENN BIOSCIENCES CORPORATION, South San Francisco, CA (US)

(72) Inventors: Paul Oluwaseyi AIYETAN, Frederick, MD (US); Matthew Peter CAMPBELL, Melbourne, Victoria (AU); Xin CONG, South San Francisco, CA (US); Garrett Anders COUTRE, Davis, CA (US); Arshia Bassi DEEP, Union City, CA (US); Monil Dilip GANDHI, South San Francisco, CA (US); Hector Han-Li HUANG, South San Francisco, CA (US); Alan Nicolas MITCHELL, South San Francisco, CA (US); Kenan PANDZA, Pleasant Hill, CA (US); Chad Eagle PICKERING, South San Francisco, CA (US); Flavio SCHWARZ, South San Francisco, CA (US); Daniel SERIE, South San Francisco, CA (US); Apoorva SRINIVASAN, San Francisco, CA (US); Saurabh SRIVASTAVA, South San Francisco, CA (US); Diane Nai-Fan TU, Pacifica, CA (US); Gege XU, South San Francisco, CA (US); Rao Siddhant YADAV, Melbourne, Victoria (AU); Bo ZHOU, Los Angeles, CA (US)

(21) Appl. No.: 18/840,561

(22) PCT Filed: Feb. 24, 2023
(86) PCT No.: PCT/US2023/063298

§ 371 Date: Aug. 22, 2024

Related U.S. Application Data
(60) Provisional application No. 63/313,690, filed on Feb. 24, 2022.
(60) Provisional application No. 63/313,693, filed on Feb. 24, 2022.
(60) Provisional application No. 63/314,274, filed on Feb. 25, 2022.
(60) Provisional application No. 63/314,895, filed on Feb. 28, 2022.
(60) Provisional application No. 63/337,933, filed on May 3, 2022.
(60) Provisional application No. 63/395,714, filed on Aug. 5, 2022.
(60) Provisional application No. 63/396,546, filed on Aug. 9, 2022.
(60) Provisional application No. 63/402,813, filed on Aug. 31, 2022.
(60) Provisional application No. 63/375,836, filed on Sep. 15, 2022.
(60) Provisional application No. 63/477,808, filed on Dec. 29, 2022.

(51) Int. Cl. G01N 33/68; G01N 30/06; G01N 33/543; G16B 40/10; G16H 50/20

(52) U.S. Cl. CPC G01N 33/6848; G01N 30/06; G01N 33/54366; G01N 33/6893; G16B 40/10; G16H 50/20; G01N 2030/067; G01N 2458/15; G01N 2800/50; G01N 2800/52

(57) ABSTRACT

A method, system, and composition related to the preparation of samples for glycoproteomic analysis is described. The sample preparation process can include a proteolytic digestion step followed by a measurement step of the glycopeptide and peptide amounts in the proteolytic digest using a liquid chromatography-mass spectrometry system. Optionally, the sample preparation process can also include the collection of the sample on an absorbent or bibulous member where the proteins and glycoproteins are later extracted and then digested for glycoproteomic analysis. Glycopeptide and peptide measurements of biological samples were analyzed to provide a diagnosis of a disease such as, for example, ovarian cancer or to assess whether a patient with melanoma is likely or not likely to benefit from checkpoint inhibitor therapy.

### CROSS-REFERENCE TO RELATED APPLICATIONS

This application claims the priority benefit of U.S. Provisional Patent Application Ser. No. 63/313,693, filed Feb. 24, 2022 [Attorney Docket No. 16653-30013.00/VENN-00022PR]; U.S. Provisional Patent Application Ser. No. 63/313,690, filed Feb. 24, 2022; [Attorney Docket No. 16653-30016.00/VENN-00026PR]; U.S. Provisional Patent Application Ser. No. 63/314,274, filed Feb. 25, 2022; [Attorney Docket No. 16653-30017.00/VENN-00027PR]; U.S. Provisional Patent Application Ser. No. 63/314,895, filed Feb. 28, 2022; [Attorney Docket No. 16653-30018.00/VENN-00030PR]; U.S. Provisional Patent Application Ser. No. 63/337,933, filed May 3, 2022; [Attorney Docket No. 16653-30021.00/VENN-00035PR]; U.S. Provisional Patent Application Ser. No. 63/395,714, filed Aug. 5, 2022; [Attorney Docket No. VENN.P0020USP1/1001219027/VENN-00046PR]; U.S. Provisional Patent Application Ser. No. 63/402,813, filed Aug. 31, 2022; [Attorney Docket No. VENN.P0020USP2/1001222750/VENN-00046P1]; U.S. Provisional Patent Application Ser. No. 63/477,808, filed Dec. 29, 2022; [Attorney Docket No. VENN-00046P2]; U.S. Provisional Patent Application Ser. No. 63/396,546, filed Aug. 9, 2022; [Attorney Docket No. 16653-30026.00/VENN-00051PR]; and U.S. Provisional Patent Application Ser. No. 63/375,836, filed Sep. 15, 2022; [Attorney Docket No. VENN-00060PR], which are hereby all incorporated by reference herein in their entirety.

### TECHNICAL FIELD

The present disclosure, in certain aspects, is directed to methods and systems, and compositions and information obtained therefrom, for preparing a proteolytic digestion of a sample comprising a glycoprotein and techniques for introducing a proteolytic digestion to a mass spectrometer. In other aspects, the sample was in an absorbent or bibulous member, such as a dried blood spot card, comprising one or more extraction internal standards comprising at least one polypeptide standard deposited thereon prior to deposition of a blood sample. In other aspects, the sample on the absorbent or bibulous member was analyzed for ovarian cancer glycopeptide biomarkers. In other aspects, a blood-derived samples (e.g., plasma) was processed to form fibrinogen-depleted samples before analyzing with liquid chromatography-mass spectrometry. In other aspects, the disclosure relates to methods and systems for analyzing peptide structures for the generation of a composite measure that represents the weighted average of each glycan monomer across glycan species. In other aspects, the composite measure was determined to predict whether a patient is not likely to benefit from checkpoint inhibitor therapy. In other aspects, the disclosure of the systems and methods relate to predicting retention times in mass spectrometry runs related to quantifying or detecting peptides in biological samples.

### BACKGROUND

Post-translational modifications of polypeptides, including polypeptide glycosylation, play vital roles in human physiology and biological signaling. The identification of aberrant glycosylation provides opportunities for early detection, intervention, and treatment of affected subjects. Current biomarker identification methods, such as those developed in the fields of proteomics and genomics, can be used to detect indicators of certain diseases, such as cancer, and to differentiate certain types of cancer from other, non-cancerous diseases. However, the use of glycoproteomic analyses has not previously been used to successfully identify disease processes. Mass spectrometry analysis has the potential to provide in-depth information of glycoproteins although the nature of glycoproteins presents many challenges not currently addressed with conventional sample preparation and mass spectrometry workflows.

For example, heterogeneity of glycosylation poses a big challenge for large-scale serum/plasma glycopeptide identification and quantitation due to low concentration of individual glycopeptides compared to unglycosylated peptides in a proteolytic digest. There is a need in the art for processing techniques to prepare proteolytic digest samples for use in liquid chromatography-mass spectrometry analysis of glycopeptides.

Robust, repeatable, and high-throughput mass spectrometry-bases analyses are challenging in view of sample source and pre-processing heterogeneity, and the diverse array and low abundance of glycosylation biomarkers having diagnostic significance. For example, there are numerous approaches for generating plasma, including those involving the use of different anticoagulants such as Streck, EDTA, Heparin or Li-Heparin, ACD, CPDA, and oxalate fluoride. The result is a non-uniform sample source, which can readily hamper biomarker development and assessment. Furthermore, biomarkers, including unique glycosylation of peptide sequences, are often very low abundance species as compared to high abundant proteins in blood-derived samples (e.g., plasma or serum) making biomarker studies difficult even using mass spectrometry. There is a need in the art for techniques to prepare blood-derived samples for use in mass spectrometry.

LC-MS analysis methods for assessing the state of an individual, such as using one or more biomarkers (e.g., a glycopolypeptide), require samples from the individual. Certain sample types that show promise for providing informative material for a LC-MS analysis are invasive, such as tissue samples (e.g., a tumor tissue), or require special sample handling to maintain the integrity of the components therein, such as liquid blood samples requiring, e.g., inhibition of enzymes and proper shipping and handling conditions.

A variety of proteolytic sample preparation protocols and kits are available for proteomic analysis. For example, RapiGest SF surfactant, S-Trap, and microwave-assisted digestion protocols have shown promise for preparing proteolytic digestions of non-glycosylated polypeptide sample. However, the inventors of the present application assessed such solutions for the analysis of glycoproteins and discovered a number of shortcomings associated with incomplete digestion (e.g., presence of missed cleavages), loss of certain glycopeptides prior to being analyzed by the mass spectrometer, biasing of certain glycopeptides, and poor reproducibility. As the use of glycoproteins in the study of human physiology requires techniques that provide a complete, accurate, quantified, and reproducible analysis of glycoproteins in a sample from an individual, there is a need in the art for new proteolytic digestion and liquid chromatography-mass spectrometry techniques for analyzing sample containing a glycoprotein.

Proteolytic digestion techniques introduce components, such as salts, reagents, and byproducts, that can lead to downstream system-based issues, e.g., contaminated mass spectrometers requiring more frequent cleaning maintenance, clogged or partially clogged components, and analytic issues leading to poor signal, poor reproducibility, and poor quantification. It is desirable to remove these components, but conventional techniques are hampered by loss of sample, especially more hydrophilic polypeptides, e.g., certain glycopeptides. There is a need in the art for new processing techniques to produce a processed sample suitable for use in liquid chromatography-mass spectrometry analysis of a sample containing a glycopolypeptide.

Glycoprotein analysis is fraught with challenges on several levels. For example, a single glycan composition in a peptide can contain a large number of isomeric structures due to different glycosidic linkages, branching patterns, and/or multiple monosaccharides having the same mass. In addition, the presence of multiple glycans that share the same peptide backbone can lead to assay signals from various glycoforms, lowering their individual abundances compared to aglycosylated peptides. Accordingly, the development of algorithms that can identify glycan structures on peptide fragments remains elusive.

In light of the above, there is a need for improved analytical methods that involve site-specific analysis of glycoproteins to obtain information about protein glycosylation patterns, which can in turn provide quantitative information that can be used to identify disease states. For example, there is a need to use such analysis to diagnose and/or treat melanoma.

An approach that is non-invasive, accurate, and reliable and that enables early diagnosis and informs treatment is needed. An approach enabling early diagnosis and informing treatment may help reduce negative health outcomes in patients with melanoma. Such an approach can assist in guiding a patient to an urgency for further testing, for example, or in guiding a medical practitioner in predicting whether a particular treatment (e.g., immunotherapy) may or may not be effective and informing treatment decisions accordingly. Thus, it may be desirable to have methods and systems capable of addressing one or more of the above-identified issues.

### BRIEF SUMMARY

### Section 1—Proteolytic Digestion and LC-MS Analysis Techniques for Samples Containing a Glycosylated Polypeptide

In some aspects, provided herein is a method for performing a liquid chromatography-mass spectrometry analysis of a proteolytic glycopeptide derived from a biological sample comprising a glycoprotein, the method comprising: subjecting the biological sample to a thermal denaturation technique to produce a denatured sample followed by a proteolytic digestion technique to produce a proteolytically digested sample comprising the glycopeptide, wherein the thermal denaturation technique subjects the biological sample to a thermal cycle comprising a thermal treatment of about 60° C. to about 100° C. with a hold time of at least about 1 minute, wherein the lid temperature during the thermal cycle is at least about 2° C. higher than the temperature of the block temperature during the thermal cycle, wherein the proteolytic digestion technique comprises adding an amount of one or more proteolytic enzymes and incubating for a digestion incubation time, and wherein the digestion technique comprises quenching the one or more proteolytic enzymes following the digestion incubation time; introducing the proteolytically digested sample to a liquid chromatography (LC) system of a LC-MS system; and performing a LC separation to introduce the proteolytic glycopeptide to a mass spectrometer (MS) system, wherein the LC separation comprises a period of diversion of an initial eluate comprising a salt, and wherein the LC system comprises a reversed-phase chromatography column.

In some embodiments, the method further comprises subjecting the denatured sample to a reduction technique followed by an alkylation technique prior to the proteolytic digestion technique. In some embodiments, the reduction technique comprises subjecting the denatured sample to a reduction technique to produce a reduced sample, wherein the reduction technique comprises adding an amount of a reducing agent to the denatured sample and incubating for a reducing incubation time. In some embodiments, the alkylation technique comprises subjecting the reduced sample to an alkylation technique to produce an alkylated sample, wherein the alkylation technique comprises adding an amount of an alkylating agent to the reduced sample and incubating substantially in in a low light condition for an alkylation incubation time, and wherein the alkylated technique comprises quenching the alkylating agent following the alkylation incubation time.

In other aspects, provided herein is a method for proteolytically digesting a biological sample comprising a glycoprotein to produce a proteolytic glycopeptide, the method comprising: subjecting the biological sample to a thermal denaturation technique to produce a denatured sample, wherein the thermal denaturation technique comprises subjecting the biological sample to a thermal cycle comprising a thermal treatment of about 60° C. to about 100° C. with a hold time of at least about 1 minute, wherein the lid temperature during the thermal cycle is at least about 2° C. higher than the temperature of the block temperature during the thermal cycle; subjecting the denatured sample to a reduction technique to produce a reduced sample, wherein the reduction technique comprises adding an amount of a reducing agent to the denatured sample and incubating for a reducing incubation time; subjecting the reduced sample to an alkylation technique to produce an alkylated sample, wherein the alkylation technique comprises adding an amount of an alkylating agent to the reduced sample and incubating substantially in the dark or in a low light condition for an alkylation incubation time, and wherein the alkylated technique comprises quenching the alkylating agent following the alkylation incubation time; and subjecting the alkylated sample to a proteolytic digestion technique to produce a proteolytically digested sample comprising the proteolytic glycopeptide, wherein the proteolytic digestion technique comprises adding an amount of one or more proteolytic enzymes and incubating for a digestion incubation time, and wherein the proteolytic digestion technique comprises quenching the one or more proteolytic enzymes following the digestion incubation time.

In some embodiments, the glycopeptide comprises a hydrophilic glycan portion. In some embodiments, the glycopeptide comprises a hydrophobic glycan portion.

In some embodiments, the biological sample is derived from a human. In some embodiments, the biological sample is a blood sample or a derivative thereof. In some embodiments, the biological sample is a plasma sample. In some embodiments, the biological sample is a serum sample. In some embodiments, the biological sample is not subjected to a high-abundant protein depletion technique prior to the thermal denaturation technique.

In some embodiments, the thermal cycle comprises a block set temperature of about 60° C. to about 100° C. with a hold time of at least about 1 minute. In some embodiments, the thermal cycle comprises a block ending temperature of about 15° C. to about 40° C. In some embodiments, the thermal cycle comprises a block starting temperature of about 15° C. to about 50° C. In some embodiments, the thermal cycle is performed in a thermal cycler comprising a lid temperature control element. In some embodiments, the thermal cycle comprises a ramp rate between the block set temperature and the block ending temperature of about 1° C./second to about 10° C./second.

In some embodiments, the proteolytic digestion technique is performed at a temperature of about 20° C. to about 55° C. In some embodiments, the digestion incubation time is at least about 20 minutes. In some embodiments, the proteolytic digestion technique is performed at a temperature of about 37° C. for at least about 12 hours. In some embodiments, the proteolytic digestion technique is performed using a second thermal cycle, wherein the lid temperature during the second thermal cycle is at least about 2° C. higher than the temperature of the block temperature during the second thermal cycle. In some embodiments, the second thermal cycle is performed in a thermal cycler comprising a lid temperature control element. In some embodiments, each of the one or more proteolytic enzymes is selected from the group consisting of trypsin and LysC. In some embodiments, the trypsin is methylated and/or acetylated. In some embodiments, the amount of the one or more proteolytic enzymes is in a proteolytic enzyme concentration to sample protein weight ratio of about 1:20 to about 1:40. In some embodiments, quenching the one or more proteolytic enzymes is performed using an acid. In some embodiments, the acid is formic acid (FA) or trifluoroacetic acid (TFA), or a mixture thereof.

In some embodiments, the reduction technique is performed at a temperature of about 35° C. to about 70° C. In some embodiments, the reduction incubation time is at least about 20 minutes. In some embodiments, the reduction technique is performed at a temperature of about 60° C. for at least about 50 minutes. In some embodiments, the reduction technique is performed using a third thermal cycle, wherein the lid temperature during the third thermal cycle is at least about 2° C. higher than the temperature of the block temperature during the third thermal cycle. In some embodiments, the third thermal cycle is performed in a thermal cycler comprising a lid temperature control element. In some embodiments, the reducing agent is dithiothreitol (DTT) or tris(2-carboxyethyl) phosphine (TCEP). In some embodiments, DTT is added in an amount of about 10 mM to about 100 mM.

In some embodiments, the alkylation technique is performed at a temperature of about 20° C. to about 37° C. In some embodiments, the alkylation incubation time is at least about 5 minutes. In some embodiments, the alkylation technique is performed at a temperature of about 20° C. to about 25° C. for at least about 30 minutes. In some embodiments, the alkylating agent is iodoacetamide (IAA). In some embodiments, IAA is added in an amount of about 10 mM to about 200 mM. In some embodiments, quenching the alkylating agent comprises use of a neutralizing agent. In some embodiments, the neutralizing agent is DTT.

In some embodiments, the proteolytically digested sample is introduced to the LC-MS system without performing an offline desalting technique.

In some embodiments, the period of diversion of the LC separation technique comprises about 1 to about 5 column volumes of the initial eluate that are diverted to waste.

In some embodiments, the LC-MS technique is a high pressure LC-MS technique. In some embodiments, the LC-MS technique comprises multiple reaction monitoring.

In some embodiments, the method further comprises adding a standard to the proteolytically digested sample prior to the LC-MS technique. In some embodiments, the standard is a stable isotope-internal standard (SI-IS) peptide mixture.

In some embodiments, the biological sample is admixed with a buffer prior to the thermal denaturation technique. In some embodiments, the buffer is ammonium bicarbonate. In some embodiments, the proteolytic glycopeptide comprises one or more sialic acid groups.

In some embodiments, the proteolytically digested sample introduced to the liquid chromatography (LC) system comprises one or more of the DTT, the IAA, the iodide, and a disulfide bonded 6-membered ring, wherein the disulfide bonded 6-membered ring is a byproduct of DTT.

### Section 2—Reversed-Phase Proteolytic Digestion Clean-Up Techniques for Samples Containing a Glycosylated Polypeptide

In certain aspects, provided herein is a method for processing a proteolytically digested sample to produce a processed sample suitable for use in a liquid chromatography-mass spectrometry (LC-MS) analysis, wherein the proteolytically digested sample comprises a plurality of proteolytic polypeptides comprising at least one proteolytic glycopeptide, the method comprising: performing one or more of the following: (a) subjecting the proteolytically digested sample to a solid phase extraction column comprising a reversed-phase medium according to one or more conditions to associate at least a portion of the plurality of proteolytic polypeptides with the reversed-phase medium, the one or more conditions comprising: (i) a polypeptide loading amount of about 50% or less of a binding capacity of the reversed-phase medium, wherein the binding capacity of the reversed-phase medium is based on an insulin load having 10% or less breakthrough; or (ii) a polypeptide loading concentration of about 0.6 μg/μL or less; or (b) subjecting the reversed-phase medium comprising the associated proteolytic polypeptides to a wash buffer at a wash flow rate of about 0.1 column volumes/minute to about 2 column volumes/minute; and subjecting the reversed-phase medium comprising the associated proteolytic polypeptides to an elution buffer to produce the processed sample.

In some embodiments, the one or more conditions to associate at least the portion of the plurality of proteolytic polypeptides with the reversed-phase medium comprises the polypeptide loading amount of about 50% or less of the binding capacity of the reversed-phase medium.

In some embodiments, the one or more conditions to associate at least the portion of the plurality of proteolytic polypeptides with the reversed-phase medium comprises the polypeptide loading concentration of about 0.6 μg/μL or less.

In some embodiments, the one or more conditions to associate at least the portion of the plurality of proteolytic polypeptides with the reversed-phase medium comprises the wash flow rate of about 0.1 column volumes/minute to about 2 column volumes/minute.

In some embodiments, the column comprising the reversed-phase material has a medium volume of about 1 to about 10 μL.

In some embodiments, the polypeptide loading amount is about 30 μg to about 200 μg. In some embodiments, the polypeptide loading amount is contained in a solution volume of at least about 100 μL.

In some embodiments, the wash flow rate is about 10 μL/minute or less.

In some embodiments, the reversed-phase medium comprises an alkyl-based moiety covalently bound to a solid phase. In some embodiments, the alkyl-based moiety comprises an octadecyl carbon functional group (C18) covalently bound to the solid phase. In some embodiments, the alkyl-based moiety comprises an octa carbon functional group (C8) covalently bound to the solid phase. In some embodiments, the carbon alkyl-based moiety comprises a tetra carbon functional group (C4) covalently bound to the solid phase. In some embodiments, the solid phase comprises a silica material.

In some embodiments, the reversed-phase medium comprises a hydrophobic polymer material. In some embodiments, the hydrophobic polymer material comprises a phenyl moiety. In some embodiments, the hydrophobic polymer material comprises a reaction product of divinylbenzene. In some embodiments, the hydrophobic polymer material comprises poly(styrene-co-divinylbenzene).

In some embodiments, the method further comprises subjecting the reversed-phase medium comprising the associated proteolytic polypeptides to a wash buffer prior to subjecting the reversed-phase medium to the elution buffer.

In some embodiments, the method further comprises subjecting the processed sample comprising the elution buffer to a drying technique to produce a dried sample.

In some embodiments, the method further comprises reconstituting the dried sample to produce a reconstituted sample and inputting the reconstituted sample into a LC chromatography system of a LC-MS system to obtain mass spectrometry data.

In some embodiments, the method further comprises identifying a polypeptide sequence of a glycopeptide from the mass spectrometry data. In some embodiments, the method further comprising identifying a glycan attachment site of the glycopeptide from the mass spectrometry data. In some embodiments, the method further comprises identifying a glycan structure of the glycopeptide from the mass spectrometry data. In some embodiments, the at least one glycopeptide comprises a glycan structure comprising one or more sialic acid moieties.

In some embodiments, the proteolytically digested sample is obtained from a method for proteolytically digesting a biological sample comprising a glycoprotein.

### Section 3—Absorbent or Bibulous Members Having a Polypeptide Standard and Configured for Deposition of a Blood Sample and LC-MS Analysis of Glycopeptides Therefrom

Provided herein is a method for performing a liquid chromatography-mass spectrometry (LC MS) analysis of a proteolytic glycopeptide derived from a blood sample deposited on a delimited zone of an absorbent or bibulous member wherein the blood sample comprises a plurality of polypeptides comprising at least one glycoprotein, the method comprising: extracting at least a portion of the plurality of polypeptides and one or more extraction internal standards from the blood spot card to obtain an extracted sample, wherein the blood spot card comprises the one or more extraction internal standards prior to deposition of the blood sample within the delimited zone, and wherein at least one of the one or more extraction internal standards comprises a polypeptide standard; subjecting the extracted sample or a derivative thereof to a proteolytic digestion technique to produce a proteolytically digested sample comprising the proteolytic glycopeptide; introducing at least a portion of the proteolytically digested sample to a liquid chromatography (LC) system of a LC-MS system; and performing the LC-MS analysis on at least the proteolytic glycopeptide and the one or more extraction internal standards.

In some embodiments, LC-MS analysis comprises measuring an abundance signal for the proteolytic glycopeptide and an abundance signal for the one or more extraction internal standards. In some embodiments, the LC-MS analysis further comprises calculating a concentration of the proteolytic glycopeptide based on a concentration of the one or more extraction internal standards prior to deposition on the blood spot card, the abundance signal for the proteolytic glycopeptide, and the abundance signal for the one or more extraction internal standards.

In some embodiments, the method comprises determining an extraction efficiency based on the LC-MS analysis of at least one of the one or more extraction internal standards. In some embodiments, the method comprises determining a digestion efficiency based on the LC-MS analysis of at least one of the one or more extraction internal standards. In some embodiments, the method comprises assessing a sample migration pattern based on the LC-MS analysis of at least one of the one or more extraction internal standards. In some embodiments, the one or more extraction internal standards comprise a plurality of polypeptide standards, and wherein at least two of the plurality of polypeptide standards have different amino acid lengths.

In some embodiments, the amino acid lengths of the plurality of polypeptide standards of the one or more extraction internal standards range from 4 amino acid to 1500 amino acids. In some embodiments, the at least one polypeptide standard of the one or more extraction internal standards comprises at least one internal enzymatic cleavage site. In some embodiments, the one or more extraction internal standards comprise a plurality of polypeptide standards, wherein at least two of the plurality of polypeptide standards have different net hydrophobicities as based on a computation tool or partition coefficient analysis. In some embodiments, the plurality of polypeptide standards have different net hydrophobicities comprises a hydrophobicity range of about-0.5 to about 1 according to the Grand average of hydropathicity index (GRAVY).

In some embodiments, the at least one polypeptide standard of the one or more extraction internal standards comprises a C-terminal arginine or lysine. In some embodiments, the at least one polypeptide standard of the one or more extraction internal standards comprises an amino acid sequence that does not have homology to a peptide derived from the human proteome. In some embodiments, the at least one polypeptide standard of the one or more extraction internal standards is a synthetic polypeptide. In some embodiments, the at least one polypeptide standard of the one or more extraction internal standards comprises a stable heavy isotope label. In some embodiments, the at least one polypeptide standard of the one or more extraction internal standards comprises a sequence that is orthogonal to an endogenous polypeptide of an individual from which the blood sample originates. In some embodiments, the at least one polypeptide standard of the one or more extraction internal standards is an analog of an endogenous polypeptide of an individual from which the blood sample originates. In some embodiments, the analog is a stable heavy isotope labeled analog. In some embodiments, the at least one polypeptide standard of the one or more extraction internal standards is a recombinantly expressed polypeptide.

In some embodiments, the at least one polypeptide standard of the one or more extraction internal standards is a glycopolypeptide. In some embodiments, the at least one polypeptide standard of the one or more extraction internal standards is a polypeptide that does not substantially interact with hemoglobin. In some embodiments, the at least one polypeptide standard of the one or more extraction internal standards comprises at least a contiguous 4 amino acid sequence from SEQ ID NOS: 1-7. In some embodiments, the at least one polypeptide standard of the one or more extraction internal standards comprises a sequence is selected from the group consisting of SEQ ID NOS: 8-9

In some embodiments, the bibulous or absorbent member is a blood spot card. In some embodiments, the blood spot card comprises a known amount of each of the one or more extraction internal standards. In some embodiments, the known amount of each of the one or more extraction internal standards is about 0.05 ppm to about 5 ppm. In some embodiments, the one or more extraction internal standards are deposited and dried on the blood spot card within an area having a surface area of about 1,000 mm² or less. In some embodiments, the one or more extraction internal standard are deposited and dried on the blood spot card within the delimited zone.

In some embodiments, extracting the at least the portion of the plurality of polypeptides and the one or more extraction internal standards from the blood spot card comprises: separating one or more portions of the blood spot card from the blood spot card, wherein the one or more portions of the blood spot card comprise at least a portion of the blood sample and the one or more extraction internal standards; extracting at least the portion of the plurality of polypeptides and the one or more extraction internal standards from the one or more portions of the blood spot card into an extraction solution; and precipitating at least the portion of the plurality of polypeptides and the one or more extraction internal standards to obtain the extracted sample.

In some embodiments, the one or more portions of the blood spot card comprises punching the one or more portion of the blood spot card using a punching device. In some embodiments, each of the one or more portions separated from the blood spot card have a surface area of about 2 mm² to about 100 mm².

In some embodiments, precipitating at least the portion of the plurality of polypeptides and the one or more extraction internal standards comprises subjecting the at least the portion of the plurality of polypeptides and the one or more extraction internal standards to ethanol.

In some embodiments, the method further comprises adding a solution to the extracted sample to resolubilize polypeptide content therein prior to subjecting the extracted sample or the derivative thereof to the proteolytic digestion technique. In some embodiments, the proteolytic digestion technique comprises a thermal denaturation technique. In some embodiments, the proteolytic digestion technique further comprises a reduction technique and an alkylation technique. In some embodiments, the proteolytic digestion technique comprises the use of one or more proteases. In some embodiments, the protease is trypsin.

In some embodiments, the LC-MS analysis comprises a multiple-reaction-monitoring (MRM) technique targeting the proteolytic glycopeptide and the one or more extraction internal standards. In some embodiments, the LC-MS analysis comprises a multiple-reaction-monitoring (MRM) technique targeting the one or more quantification internal standards. In some embodiments, the absorbent or bibulous member (such as a blood spot card) comprises a delimited zone having a surface area of about 1,000 mm² or less. In some embodiments, the absorbent or bibulous member (such as a blood spot card) comprises a filter paper material. In some embodiments, the filter paper material comprises a cellulose-based paper. In some embodiments, the filter paper material prevents or reduces sample hemolysis.

In some embodiments, the absorbent or bibulous member (such as a blood spot card) comprises a lateral flow material configured to separate whole blood into a portion of plasma, wherein the whole blood is deposited at the delimited zone and then a liquid portion of the whole blood laterally flows from the delimited zone to a distal zone, wherein the distal zone contains the portion of the plasma.

Also provided herein is an absorbent or bibulous member (such as a blood spot card) comprising one or more extraction internal standard deposited thereon on a delimited zone, wherein the one or more extraction internal standards comprises at least one polypeptide standard, and wherein the absorbent or bibulous member does not comprise a blood sample deposited thereon.

### Section 4—Method of Diagnosing Pelvic Tumors

Provided herein is a method of classifying a biological sample obtained from a subject with respect to a plurality of states associated with a pelvic cancer, the method comprising receiving peptide structure data corresponding to a set of glycoproteins in the biological sample; inputting quantification data identified from the peptide structure data for a set of peptide structures into a machine-learning model trained to identify a disease indicator based on the quantification data, wherein the set of peptide structures comprises at least one peptide structure identified from a plurality of peptide structures in Table 9; identifying, by the machine-learning model, the disease indicator; and classifying the biological sample with respect to a plurality of states associated with pelvic cancer based upon the identified disease indicator.

Also provided herein is a method of detecting the presence of one of a plurality of states associated with a pelvic cancer in a subject, the method comprising receiving peptide structure data corresponding to a set of glycoproteins in a biological sample obtained from a subject, wherein the peptide structure data comprises at least one peptide structure from Table 9; inputting quantification data identified from the peptide structure data for a set of peptide structures into a machine-learning model trained to identify a disease indicator based on the quantification data; and detecting the presence of a corresponding state of the plurality of states associated with the pelvic cancer in response to a determination that the identified disease indicator falls within a selected range associated with the corresponding state.

In some embodiments, the plurality of states comprises at least one of a malignant tumor or a benign tumor. In some embodiments, the machine-learning model comprises a logistic regression model. In some embodiments, the method further comprises administering to the subject an effective amount of a therapeutic agent to treat the pelvic tumor. In some embodiments, the pelvic tumor is ovarian cancer.

In some embodiments, provided herein is a method of treating a pelvic tumor in a subject comprising receiving peptide structure data corresponding to a set of glycoproteins in a biological sample obtained from a subject, wherein the peptide structure data comprises at least one peptide structure from Table 9; inputting quantification data for the at least one peptide structure into a machine-learning model trained to generate a risk score based on the quantification data; outputting, by the machine-learning model, the quantification data using the machine learning model to generate a risk score, administering an effective amount of an agent to treat the pelvic cancer based upon the risk score.

In some embodiments, provided herein is method of determining a diagnosis for a pelvic tumor in a subject comprising receiving peptide structure data corresponding to a set of glycoproteins in a biological sample; inputting quantification data identified from the peptide structure data for a set of peptide structures into a machine-learning model trained to identify a disease indicator based on the quantification data, wherein the set of peptide structure data comprises at least one peptide structure identified from a plurality of peptide structures in Table 9; identifying, by the machine-learning model, the disease indicator; and determining a diagnosis for the pelvic tumor based upon the identified disease indicator. In some embodiments, the diagnosis is the presence of a malignant tumor or a benign tumor.

Also provided herein is a method of treating a pelvic tumor in a subject comprising receiving peptide structure data corresponding to a set of glycoproteins in a biological sample; inputting quantification data identified from the peptide structure data for a set of peptide structures into a machine-learning model trained to identify a disease indicator based on the quantification data, wherein the peptide structure data comprises at least one peptide structure identified from a plurality of peptide structures in Table 9; identifying, by the machine-learning model, the disease indicator; determining a risk score the identified disease indicator; and administering an effective amount of an agent to treat the pelvic tumor based upon the risk score.

In some embodiments, provided herein is a method of treating a pelvic tumor in an individual comprising detecting the presence or amount of at least one peptide structure, wherein the at least one peptide structure comprises at least one peptide structure from Table 9, and administering an effective amount of a therapeutic agent to treat the pelvic tumor based upon the presence or amount of the peptide structure.

In some embodiments, provided herein is a method of diagnosing an individual with a benign or malignant pelvic tumor comprising detecting a presence or amount of at least one peptide structure, wherein the at least one peptide structure comprises at least one peptide structure from Table 9, and diagnosing the individual with a benign or malignant pelvic tumor based upon the presence or amount of the at least one peptide structure.

In some embodiments, provided herein is a method of diagnosing an individual with a pelvic tumor comprising detecting the presence or amount of at least one peptide structure from Table 9; inputting a quantification of the detected at least one peptide structure into a machine-learning model trained to generate a class label, determining if the class label is above or below a threshold for a classification; identifying a diagnostic classification for the individual based on whether the class label is above or below a threshold for the classification; and diagnosing the individual as having a benign or malignant pelvic tumor on the diagnostic classification.

In some embodiments, the method further comprises detecting the presence or amount of at least one peptide structure from Table 9. In some embodiments, the presence or amount of the at least one peptide structure is detected using mass spectrometry or ELISA. In some embodiments, the presence or amount of the at least one peptide structure is detected using MRM mass spectrometry. In some embodiments, the amount of at least one peptide structure is none, or below a detection limit.

In some embodiments, the at least one peptide structure comprises two or more peptide structures identified in Table 9, three or more peptides structures identified in Table 9, four or more peptide structure identified in Table 9, five or more peptide structures identified in Table 9, six or more peptide structures identified in Table 9, seven or more peptide structures identified in Table 9, or eight or more peptide structure identified in Table 9. In some embodiments, the at least one peptide structure comprises the sequence set forth in SEQ ID NOs: 35-51. In some embodiments, the at least one peptide structure comprises the sequence set forth in SEQ ID NOs: 35-42. In some embodiments, the at least one peptide structure comprises the sequence set forth in SEQ ID NOs: 43-51. In some embodiments, the at least one peptide structure comprises the sequence set forth in SEQ ID NOs: 35-40.

In some embodiments, the biological sample is a blood sample, a serum sample, or tumor tissue. In some embodiments, the biological sample is the blood sample, wherein the blood sample is deposited on a delimited zone of an absorbent or bibulous member comprising a plurality of polypeptides comprising at least one glycoprotein.

In some embodiments, the method further comprises extracting at least a portion of the plurality of polypeptides and one or more extraction internal standards from the absorbent or bibulous member to obtain an extracted sample, wherein the absorbent or bibulous member comprises the one or more extraction internal standards prior to deposition of the blood sample within the delimited zone, and wherein at least one of the one or more extraction internal standards comprises a polypeptide standard; subjecting the extracted sample or a derivative thereof to a proteolytic digestion technique to produce a proteolytically digested sample comprising the proteolytic glycopeptide; introducing at least a portion of the proteolytically digested sample to a liquid chromatography (LC) system of a LC-MS system; and performing the LC-MS analysis on at least the proteolytic glycopeptide and the one or more extraction internal standards, wherein the at least one proteolytic glycopeptide comprises at least one peptide structure set forth in Table 9.

Also provided herein is a method for performing a liquid chromatography-mass spectrometry (LC-MS) analysis of a proteolytic glycopeptide derived from a blood sample from an individual deposited on a delimited zone of a blood spot card, the method comprising obtaining a blood spot card comprising a blood sample from the individual deposited thereon, wherein the blood spot card comprises one or more extraction internal standards deposited and dried prior to deposition of the blood sample on the blood spot card, and wherein the blood spot card comprising the blood sample contains at least a portion of the blood sample and the one or more extraction internal standards in an overlapping area of the blood spot card; extracting at least a portion of the plurality of polypeptides and the one or more extraction internal standards from the blood spot card to obtain an extracted sample; subjecting the extracted sample or a derivative thereof to a proteolytic digestion technique to produce a proteolytically digested sample comprising the proteolytic glycopeptide; introducing at least a portion of the proteolytically digested sample to a liquid chromatography (LC) system of a LC-MS system; and performing an LC-MS analysis to quantify one or more biomarkers of ovarian cancer and the one or more extraction internal standards, wherein the one or more biomarkers comprise a polypeptide comprising a sequence of any of SEQ ID NOs: 35-51, and wherein at least one of the one or more biomarkers is a glycopeptide. In some embodiments, the at least one polypeptide standard of the one or more extraction internal standards comprises at least a contiguous 4 amino acid sequence from SEQ ID NOs: 14-20. In some embodiments, the at least one polypeptide standard of the one or more extraction internal standards comprises a sequence is selected from the group consisting of SEQ ID NOs: 21-22. In some embodiments, wherein the absorbent or bibulous member comprises a known amount of each of the one or more extraction internal standards.

In some embodiments, extracting the at least the portion of the plurality of polypeptides and the one or more extraction internal standards from the absorbent or bibulous member comprises separating one or more portions of the absorbent or bibulous member from the absorbent or bibulous member, wherein the one or more portions of the absorbent or bibulous member comprise at least a portion of the blood sample and the one or more extraction internal standards; extracting at least the portion of the plurality of polypeptides and the one or more extraction internal standards from the one or more portions of the absorbent or bibulous member into an extraction solution; and precipitating at least the portion of the plurality of polypeptides and the one or more extraction internal standards to obtain the extracted sample.

In some embodiments, the precipitating at least the portion of the plurality of polypeptides and the one or more extraction internal standards comprises subjecting at least the portion of the plurality of polypeptides and the one or more extraction internal standards to either ethanol, methanol, or acetone.

In some embodiments, the method, further comprises adding a solution to the extracted sample to resolubilize polypeptide content therein prior to subjecting the extracted sample or the derivative thereof to the proteolytic digestion technique. In some embodiments, the proteolytic digestion technique comprises a thermal denaturation technique. In some embodiments, the proteolytic digestion technique further comprises a reduction technique and an alkylation technique. In some embodiments, the proteolytic digestion technique comprises the use of one or more proteases. In some embodiments, the protease is trypsin.

In some embodiments, the absorbent or bibulous member comprises a filter paper material. In some embodiments, the filter paper material comprises a cellulose-based paper. In some embodiments, the filter paper material prevents or reduces sample hemolysis.

In some embodiments, the absorbent or bibulous member comprises a lateral flow material configured to separate whole blood into a portion of plasma, wherein the whole blood is deposited at the delimited zone and then a liquid portion of the whole blood laterally flows from the delimited zone to a distal zone, wherein the distal zone contains the portion of the plasma.

Also provided herein is a method of training a model to diagnose a subject with one of a plurality of states associated with a pelvic tumor, the method comprising receiving quantification data for a panel of peptide structures for a plurality of subjects diagnosed with the plurality of states associated with a pelvic tumor wherein the panel of peptide structures comprises at least one peptide structure set forth in Table 9; and training a machine-learning model to determine a state of the plurality of states a biological sample from the subject based on the quantification data.

In some embodiments, the quantification data comprises at least one of an abundance, a relative abundance, a normalized abundance, a relative quantity, an adjusted quantity, a normalized quantity, a relative concentration, an adjusted concentration, or a normalized concentration.

In some embodiments, the machine-learning model is trained using random forest or logical progression training methods.

In some embodiments, training the machine-learning model to determine the state of the plurality of states comprises training the machine-learning model to generate a class label for the state of the plurality of states.

In some embodiments, the machine-learning model comprises a logistic regression model.

In some embodiments, at least one of the peptide structures comprises a glycopeptide.

Also provided herein is a composition comprising one or more peptide structure from Table 9.

Also provided herein is a composition comprising one or more peptides comprising the sequence set forth in SEQ ID NOs: 35-51.

### Section 5—HILIC Enrichment Sample Preparation for Quantitative Mass Spectrometry

In certain aspects, provided is a method for processing a proteolytic digest sample for use in a liquid chromatography-mass spectrometry (LC-MS) analysis, wherein the proteolytic digest sample comprises a plurality of proteolytically digested peptides comprising at least one proteolytically digested glycopeptide, the method comprising: (A) loading a hydrophilic interaction liquid chromatography (HILIC) load derived from the proteolytic digest sample to a solid phase extraction column comprising a HILIC medium according to one or more conditions to associate the at least one proteolytically digested glycopeptide with the HILIC medium, the one or more conditions comprising: (1) the loading of the HILIC load to the solid phase extraction column is initiated when the HILIC medium is in a dry state; (2) the HILIC load loaded to the solid phase extraction column has an amount of the plurality of proteolytically digested peptides characterized by one or both of: (a) a ratio of the weight of the plurality of proteolytically digested peptides over the weight of the HILIC medium in the dry state of at least about 0.06; and/or (b) a ratio of the weight of the plurality of proteolytically digested peptides relative to the bed volume of the HILIC medium in the dry state of at least about 40 μg/μl; or (3) the HILIC load loaded to the solid phase extraction column has a concentration of an organic solvent of at least about 70% (v/v); and (B) subjecting the HILIC medium to an elution liquid to obtain a HILIC eluate comprising the at least one proteolytically digested glycopeptide.

In some embodiments, the one or more loading conditions comprise the loading of the HILIC load to the solid phase extraction column being initiated when the HILIC medium is in the dry state.

In some embodiments, the HILIC load is characterized by having a ratio of the weight of the plurality of proteolytically digested peptides over the weight of the HILIC medium in the dry state of at least about 0.06. In some embodiments, the HILIC load is characterized by having a ratio of the weight of the plurality of proteolytically digested peptides relative to the bed volume of the HILIC medium in the dry state of at least about 40 μg/μl. In some embodiments, the weight of the HILIC medium in the dry state is about 3 mg or the bed volume of the HILIC medium in the dry state is about 5 μL. In some embodiments, the HILIC load is characterized by having a ratio of the weight of the plurality of proteolytically digested peptides over the weight of the HILIC medium in the dry state of about 0.1. In some embodiments, the HILIC load is characterized by having a ratio of the weight of the plurality of proteolytically digested peptides relative to the bed volume of the HILIC medium in the dry state of about 60 μg/μl. In some embodiments, the HILIC load is characterized by having a ratio of the weight of the plurality of proteolytically digested peptides over the weight of the HILIC medium in the dry state of about 0.2. In some embodiments, the HILIC load is characterized by having a ratio of the weight of the plurality of proteolytically digested peptides relative to the bed volume of the HILIC medium in the dry state of about 120 μg/μl.

In some embodiments, the one or more conditions comprise the HILIC load loaded to the solid phase extraction column having a concentration of the organic solvent of at least about 70% (v/v).

In some embodiments, the HILIC medium comprises less than about 5% (v/v) of a liquid at the initiation of the loading of the HILIC load to the solid phase extraction column. In some embodiments, wherein, at the initiation of the loading of the HILIC load to the HILIC medium of the solid phase extraction column, the HILIC medium is not equilibrated with an equilibration liquid.

In some embodiments, the HILIC load comprises an amount of the plurality of proteolytically digested peptides of at least about 200 μg.

In some embodiments, the concentration of the organic solvent in the HILIC load is at least about 80% (v/v). In some embodiments, the organic solvent comprises an aprotic solvent miscible in water. In some embodiments, the organic solvent is selected from the group consisting of acetonitrile, ethanol, methanol, tetrahydrofuran, and dioxane, or a combination thereof.

In some embodiments, the method further comprises obtaining the HILIC load. In some embodiments, the obtaining the HILIC load comprises reducing a liquid content from the proteolytic digest sample without substantial loss of the plurality of proteolytically digested peptides in the proteolytic digest sample. In some embodiments, the reducing the liquid content from the proteolytic digested sample comprises performing a peptide concentrating technique with the proteolytically digested sample to obtain a precursor of the HILIC load such that (a) the precursor can be reconstituted with a reconstitution liquid comprising the organic solvent to obtain the HILIC load having a volume of 220 μL or less and a concentration of the organic solvent of at least about 70% (v/v); and (b) the resulting HILIC load comprises an amount of the plurality of proteolytically digested peptides of at least about 200 μg.

In some embodiments, the method further comprises: reducing a liquid content from the proteolytic digest sample to form a dried proteolytic digest sample; and reconstituting the dried proteolytic digest sample with a reconstitution liquid comprising the organic solvent to produce the HILIC load such that (a) the HILIC load has a volume of 220 μL or less and a concentration of the organic solvent of at least about 70% (v/v); and (b) the HILIC load has an amount of the plurality of proteolytic peptides of at least about 200 μg.

In some embodiments, the reconstituting the dried proteolytic digest sample comprises: mixing the dried proteolytic digest sample with an amount of water to form a water mixture: sonicating the water mixture with a sonicator; mixing the water mixture with an amount of trifluoracetic acid (TFA) and acetonitrile (ACN), wherein the amount of TFA and ACN are such that the final concentration of TFA is 1% (v/v) and the final concentration of ACN is 80% (v/v); and sonicating the water mixture having the amount of TFA and ACN with a sonicator to produce the HILIC load. In some embodiments, the sonicating the water mixture with the sonicator comprises a water-based dissolution cycle, wherein the water-based dissolution cycle is repeated about 2 times to about 5 times, and wherein for each of the water-based dissolution cycles, the sonicating the water mixture is performed for about 5 minutes and a water reservoir of the sonicator is configured with ice to cool the water reservoir. In some embodiments, the sonicating the water mixture having the amount of TFA and ACN with the sonicator comprises an organic-based dissolution cycle, wherein the organic-based dissolution cycle is repeated about 2 times to about 3 times, and wherein for each of the organic-based dissolution cycles, the sonicating is performed for about 4 minutes and a water reservoir of the sonicator is configured with ice to cool the water reservoir.

In some embodiments, the reducing the liquid content from the proteolytic digest sample comprises removing all or substantially all of the liquid content therefrom.

In some embodiments, the peptide concentrating technique comprises a vacuum evaporation technique or a lyophilization technique.

In some embodiments, the volume of the HILIC load is 220 μL or less.

In some embodiments, the HILIC medium comprises a solid phase or a solid phase comprising a polar functional moiety. In some embodiments, the solid phase comprises a silica material. In some embodiments, the polar functional moiety comprises one or more of an amino group, a cyano group, a carbamoyl group, an aminoalkyl group, alkylamide group, or a combination thereof.

In some embodiments, the method further comprises performing a washing step after loading the HILIC load to the solid phase extraction column and prior to the subjecting the HILIC medium to the elution liquid, wherein the washing step comprises subjecting the HILIC medium to a wash liquid.

In some embodiments, the method further comprises collecting the HILIC eluate, or a fraction thereof, from the solid phase extraction column, wherein the HILIC eluate comprises the at least one proteolytically digested glycopeptide. In some embodiments, after the collecting the HILIC eluate from the solid phase extraction column, the method further comprises reducing a liquid content of the collected HILIC eluate.

In some embodiments, the method further comprises subjecting the HILIC eluate to a peptide concentrating technique to produce a dried HILIC eluate.

In some embodiments, the method further comprises reconstituting the dried HILIC eluate to form a sample suitable for introduction to the LC-MS system.

In some embodiments, the method further comprises injecting the sample suitable for introduction to the LC-MS system into the LC-MS system.

In some embodiments, the method further comprises performing a mass spectrometry technique to obtain mass spectrometry data.

In some embodiments, the method further comprises identifying a peptide sequence of a glycopeptide from the mass spectrometry data.

In some embodiments, the method further comprises identifying a glycan attachment site of the glycopeptide from the mass spectrometry data.

In some embodiments, the method further comprises identifying a glycan structure of the glycopeptide from the mass spectrometry data.

In some embodiments, the at least one glycopeptide comprises a glycan structure comprising one or more sialic acid moieties.

In some embodiments, the proteolytic digest sample is obtained from a method for proteolytically digesting a biological sample comprising a glycoprotein.

In some embodiments, wherein a glycopeptide concentration for a glycopeptide derived from the proteolytic digest sample is enriched by a factor of 30 or greater with respect to a peptide concentration, wherein the peptide concentration represents an amount of a peptide that is associated with the same protein as the glycopeptide.

In some embodiments, the method further comprises: measuring a first plurality of peak area values for a first panel of glycopeptides; measuring a second plurality of peak area values for a second panel of unglycosylated peptides wherein each of the unglycosylated peptides of the second panel corresponds to each of the glycopeptides of the first panel by being attached to a same protein molecule before a proteolytic digestion; calculating a plurality of ratios by dividing each of the first plurality of peak area values with each of the second plurality of peak area values, respectively; and determining a median ratio from the plurality of ratios, wherein the median ratio is greater than 30.

### Section 6—Fibrinogen-Depletion and Use Thereof in Glycoproteomic Analysis

In certain aspects, provided herein is a method of processing a blood-derived sample obtained from an individual for a glycoproteomic mass spectrometry (MS) technique, the method comprising: (a) admixing the blood-derived sample with one or more defibrination factors to promote formation of a fibrin clot, the one or more defibrination factors comprises one or more members selected from the group consisting of: a clotting co-factor; a clotting enzyme; and a clotting activator and/or an exogenous surface aggregation agent; (b) separating the formed fibrin clot from the admixed blood-derived sample to obtain a fibrinogen-depleted sample; and (c) subjecting the fibrinogen-depleted sample to one or more MS preparation techniques to produce a test sample for the glycoproteomic mass spectrometry technique.

In some embodiments, the one or more defibrination factors comprises a clotting co-factor. In some embodiments, the clotting co-factor comprises a divalent cation. In some embodiments, the clotting co-factor comprises the divalent cation, and wherein the divalent cation is Ca²⁺, Mg²⁺, Zn²⁺, or Cu²⁺, or any combination thereof. In some embodiments, the divalent cation is Ca²⁺. In some embodiments, the clotting co-factor is calcium chloride, calcium acetate, calcium carbonate, calcium citrate, or calcium gluconate, or any combination thereof. In some embodiments, following admixing with the blood-derived sample, the clotting co-factor has a concentration of about 5 mM to about 25 mM.

In some embodiments, the one or more defibrination factors comprises a clotting enzyme. In some embodiments, the clotting enzyme is thrombin. In some embodiments, following admixing with the blood-derived sample, the clotting enzyme has a concentration of about 1 unit/mL to 10 units/mL.

In some embodiments, the one or more defibrination factors comprises a clotting activator and/or the exogenous surface aggregation agent. In some embodiments, the clotting activator and/or the exogenous surface aggregation agent is an exogenous surface aggregation agent. In some embodiments, the exogenous surface aggregation agent comprises Kaolin. In some embodiments, the clotting activator and/or the exogenous surface aggregation agent is a clotting activator and exogenous surface aggregation agent. In some embodiments, the clotting activator and exogenous surface aggregation agent comprises a material having pores with an average size of about 2 nm to about 60 nm. In some embodiments, the clotting activator and exogenous surface aggregation agent comprises a silica particle. In some embodiments, the silica particle has a pore size ranging from about 2 to about 60 nm. In some embodiments, the clotting activator and/or the exogenous surface aggregation agent is admixed with the blood-derived sample at an amount of about 50 μg to about 500 μg per 40 μL of the blood-derived sample.

In some embodiments, the one or more defibrination factors comprise the clotting co-factor and the clotting enzyme.

In some embodiments, the one or more defibrination factors comprise the clotting co-factor and the clotting activator and/or the exogenous surface aggregation agent.

In some embodiments, the one or more defibrination factors comprise the clotting enzyme and the clotting activator and/or the exogenous surface aggregation agent.

In some embodiments, the one or more defibrination factors comprise the clotting co-factor, the clotting enzyme, and the clotting activator and/or the exogenous surface aggregation agent.

In some embodiments, more than one defibrination factor is admixed with the blood-derived sample sequentially. In some embodiments, more than one defibrination factor is admixed with the blood-derived sample simultaneously. In some embodiments, at least one of the one or more defibrination factors is added to a vessel containing the blood-derived sample. In some embodiments, the blood-derived sample is added to a vessel containing at least one of the one or more defibrination factors.

In some embodiments, the method further comprises an incubation period following the admixing of the blood-derived sample with one or more defibrination factors. In some embodiments, the incubation period is about 1 minute to about 30 minutes.

In some embodiments, the separating the formed fibrin clot to obtain the fibrinogen-depleted sample comprises subjecting the admixed blood-derived sample with the one or more defibrination factors to a centrifugation technique and/or a filtration technique. In some embodiments, the separating the formed fibrin clot to obtain the fibrinogen-depleted sample comprises subjecting the admixed blood-derived sample with the one or more defibrination factors to a supernatant collection technique.

In some embodiments, the fibrinogen-depleted sample is depleted of at least about 80% of the fibrinogen as compared to the blood-derived sample.

In some embodiments, the fibrinogen-depleted sample is depleted of at least about 99% of the fibrinogen as compared to the blood-derived sample.

In some embodiments, the blood-derived sample is a plasma sample. In some embodiments, the plasma sample has been treated with an anticoagulant. In some embodiments, the plasma sample has been treated with any one or more of the following: a citrate, an ACD (anticoagulant citrate dextrose), Streck, EDTA (ethylenediaminetetraacetic acid), Heparin or Li-Heparin, oxalate fluoride, or a citrate phosphate dextrose adenine (CPDA). In some embodiments, the blood-derived sample is a serum sample. In some embodiments, the blood-derived sample is obtained from a mammal, such as a human. In some embodiments, the blood-derived sample is from a single individual, such as a single human. In some embodiments, the blood-derived sample is from a single draw from an individual. In some embodiments, the blood-derived sample is a pooled sample, such as from one or more draws from an individual and/or from one or more individuals.

In some embodiments, the one or more MS preparation techniques comprises subjecting the fibrinogen-depleted sample, or a derivative thereof, to a thermal denaturation technique. In some embodiments, the one or more MS preparation techniques comprises subjecting the fibrinogen-depleted sample, or a derivative thereof, to a proteolytic digestion technique. In some embodiments, the proteolytic digestion technique comprises the use of one or more proteases. In some embodiments, the proteolytic digestion technique comprises the use of trypsin. In some embodiments, the one or more proteases are present at a weight ratio of about 1:30 or less, relative to polypeptide content of the fibrinogen-depleted sample, or a derivative thereof. In some embodiments, the one or more MS preparation techniques comprises subjecting the fibrinogen-depleted sample, or a derivative thereof, to a desalting technique. In some embodiments, the method further comprises performing a glycoproteomic mass spectrometry technique. In some embodiments, the glycoproteomic mass spectrometry technique comprises a liquid chromatography-mass spectrometry (MS) (LC-MS) technique. In some embodiments, the LC-MS technique comprises a period of diversion of an initial eluate comprising a salt. In some embodiments, the glycoproteomic mass spectrometry technique comprises a multiple-reaction-monitoring (MRM) technique targeting a glycopeptide.

In certain aspects, provided herein is a method of preparing a plasma sample obtained from an individual (such as a human) for a glycoproteomic mass spectrometry technique, the method comprising: (a) admixing the plasma sample with defibrination factors to promote formation of a fibrin clot, the defibrination factors comprising: a clotting co-factor; a clotting enzyme; and a clotting activator and/or an exogenous surface aggregation agent; (b) separating the formed fibrin clot from the admixed plasma sample to obtain a fibrinogen-depleted sample; and (c) subjecting the fibrinogen-depleted sample to one or more MS preparation techniques to produce a test sample for the glycoproteomic mass spectrometry technique. In some embodiments, following admixing with the blood-derived sample: the clotting co-factor comprises Ca2+ at a concentration of about 5 mM to about 25 mM; the clotting enzyme comprises thrombin at a concentration of about 1 unit/mL to 10 units/mL; and the clotting activator and/or the exogenous surface aggregation agent is in an amount of about 50 μg to about 500 μg per 40 μL of the blood-derived sample.

In certain aspects, provided herein is a defibrination composition comprising: a clotting co-factor; a clotting enzyme; and a clotting activator and/or an exogenous surface aggregation agent.

In certain aspects, provided herein is a vessel (such as a sample tube) comprising any defibrination composition described herein.

### Section 7—Methods and Systems for Analyzing Site-Specific Monomer Composition

Aspects of the present disclosure are based, at least in part, on the development of methods and systems for analysis of site-specific glycan monomer composition, as well as on the discovery that such analysis can be used to predict, diagnose, prognose, and/or inform treatment of one or more disease states such as melanoma. Accordingly, aspects of the disclosure are directed to methods for analyzing a set of peptide structures for calculating one or more monomer weight scores. Also disclosed are methods for classifying a biological sample comprising analyzing monomer weight scores to generate a disease indicator and generating a diagnosis or prognosis output based on the disease indicator. Further disclosed are treatment methods comprising treatment of a melanoma subject with immunotherapy (e.g., immune checkpoint blockade therapy such as ipilimumab, nivolumab, and/or pembrolizumab) based on analysis of monomer weight scores from a biological sample from the subject.

Disclosed herein, in some aspects, is a method for analyzing a set of peptide structures comprising a linking site, the method comprising: A) calculating a site occupancy score, for a given peptide structure at the linking site, as a function of an adjusted-raw abundance value for the given peptide structure and a sum of a set of adjusted-raw abundance values of the set of peptide structures; and B) calculating a monomer weight score as a sum of the site occupancy score and a multiplier, wherein the multiplier is the number of a specific monomer in the set of peptide structures at the linking site. In some aspects, the method further comprises, prior to (A), receiving a set of raw abundance values of the set of peptide structures and normalizing the set of raw abundance values to a corresponding reference run to generate the set of adjusted-raw abundance values. In some aspects, the method further comprises, prior to (B), calculating a peptide structure monomer weight score as a function of the site occupancy score and the number of a specific monomer for the given peptide structure. In some aspects, the monomer weight score is a function of the peptide structure monomer weight score and the site occupancy score. In some aspects, the set of peptide structures is from a biological sample from a subject. In some aspects, the biological sample comprises serum or plasma samples. In some aspects, the reference run comprises serum or plasma samples. In some aspects, the method further comprises correlating the monomer weight score with an indication or disease state to determine a hazard ratio for the indication or disease state, wherein the hazard ratio is used to update a risk profile of the subject for the indication or disease state. In some aspects, the method further comprises generating a diagnosis output for the indication or disease state for the subject, using a predictive model, as a function of the monomer weight score, wherein the diagnosis output is one of a predictive probability or a risk score. In some aspects, further comprising calculating a site occupancy score, for a given peptide structure at the linking site, as the quotient of the adjusted-raw abundance value for the given peptide structure over the sum of the set of adjusted-raw abundance values. In some aspects, the method further comprises calculating a peptide structure monomer weight score as a product of the site occupancy score and the number of specific monomers for the given peptide structure. In some aspects, the method further comprises calculating a monomer weight score for the subject as a sum of peptide structure monomer weight scores for each peptide structure at the linking site. In some aspects, the method further comprises generating a diagnosis output, based on the monomer weight score, for an indication or disease state, wherein the diagnosis output classifies the biological sample as evidencing a state associated with a disease state progression and/or responsiveness to a specific therapy. In some aspects, the set of raw abundance values is generated using multiple reaction monitoring mass spectrometry (MRM-MS). In some aspects, the method further comprises generating a diagnosis output based on the monomer weight score for an indication or disease state, and generating a treatment output based on at least one of the diagnosis output. In some aspects, the treatment output comprises at least one of an identification of a treatment to treat the subject or a treatment plan. In some aspects, the treatment comprises at least one of radiation therapy, chemoradiotherapy, surgery, immunotherapy, hormone therapy, or a targeted drug therapy. In some aspects, the treatment comprises immunotherapy, wherein the immunotherapy is immune checkpoint blockade therapy. In some aspects, the immune checkpoint blockade therapy comprises ipilimumab, nivolumab, and/or pembrolizumab. In some aspects, the method further comprises generating a diagnosis output, wherein generating the diagnosis output comprises: generating a report identifying that the biological sample evidences the indication or disease state. In some aspects, the specific monomer is selected from the group consisting of hexose, HexNac, fucose, and sialic acid. In some aspects, the specific monomer is selected from the group consisting of glucose, mannose, galactose, GlcNAc, GalNAc, fucose, NeuGc, and NeuAc. In some aspects, the method further comprises calculating a second monomer weight score as a sum of the site occupancy score and a second multiplier, wherein the second multiplier is the number of a second monomer in the set of peptide structures at the linking site, wherein the second monomer is different from the specific monomer. In some aspects, the method further comprises calculating a plurality of additional monomer weight scores as functions of the site occupancy score and a plurality of additional multipliers, wherein the plurality of additional multipliers are the number of a plurality of additional monomers in the set of peptide structures at the linking site.

Disclosed herein, in some aspects, is a method of classifying a biological sample with respect to risk of melanoma progression and/or responsiveness to immune checkpoint inhibitor therapy, the method comprising: A) analyzing one or more monomer weight scores of a set of peptide structures from a biological sample from the subject using a machine learning model to generate a disease indicator; and B) generating a diagnosis output based on the disease indicator that classifies the biological sample as evidencing a state associated with melanoma progression and/or responsiveness to immune checkpoint inhibitory therapy. In some aspects, the method further comprises receiving a set of raw abundance values of the set of peptide structures and normalizing the set of raw abundance values to a corresponding reference run to generate the set of adjusted-raw abundance values. In some aspects, the method further comprises calculating a site occupancy score, for a given peptide structure at the linking site, as the function of the adjusted-raw abundance value for the given peptide structure and the sum of the set of adjusted-raw abundance values. In some aspects, the method further comprises calculating a site occupancy score, for a given peptide structure at the linking site, as the quotient of the adjusted-raw abundance value for the given peptide structure over the sum of the set of adjusted-raw abundance values calculating a peptide structure monomer weight score as a function of the site occupancy score and the number of specific monomers for the given peptide structure. In some aspects, the method further comprises calculating a peptide structure monomer weight score as a product of the site occupancy score and the number of specific monomers for the given peptide structure. In some aspects, the method further comprises calculating a monomer weight score of the one or more monomer weight scores as a sum of peptide structure monomer weight scores for each peptide structure at the linking site. In some aspects, the set of peptide structures comprises post translationally modified (PTM) peptides and/or non-PTM peptides. In some aspects, the monomer is selected from the group consisting of hexose, HexNac, fucose, and sialic acid. In some aspects, the monomer is selected from the group consisting of glucose, mannose, galactose, GlcNAc, GalNAc, fucose, NeuGc, and NeuAc. In some aspects, the set of peptides structures comprises glycosylated peptides and non-glycosylated peptides. In some aspects, the biological sample comprises serum or plasma samples. In some aspects, the reference run comprises serum or plasma samples. In some aspects, the method further comprises treating the biological sample to form a prepared sample comprising the set of peptide structures, the set of peptide structures comprising a set of post translationally modified (PTM) peptides and/or non-PTM peptides; detecting a set of product ions associated with each structure of the set of post translationally modified (PTM) peptides and/or non-PTM peptides, and generating the set of raw abundance values for the set of product ions. In some aspects, the analyzing further comprises: correlating the monomer weight score with a melanoma disease state to determine a hazard ratio for the melanoma disease state, wherein the hazard ratio is used to update a risk profile of the subject for the melanoma disease state. In some aspects, the method further comprises generating a diagnosis output based on the disease indicator that classifies the biological sample as evidencing a state associated with melanoma progression and/or responsiveness to immune checkpoint inhibitory therapy, wherein the diagnosis output is one of a predictive probability or a risk score. In some aspects, the set of raw abundance values is generated using multiple reaction monitoring mass spectrometry (MRM-MS). In some aspects, the method further comprises generating a treatment output based on at least one of the diagnosis output. In some aspects, the treatment output comprises at least one of an identification of a treatment to treat the subject or a treatment plan. In some aspects, the treatment comprises at least one of radiation therapy, chemoradiotherapy, surgery, hormone therapy, or a targeted drug therapy. In some aspects, generating the diagnosis output comprises: generating a report identifying that the biological sample evidences the indication or disease state. In some aspects, the one or more monomer weight scores correspond to at least one site monomer identified in Table 16. In some aspects, the one or more monomer weight scores correspond to at least one site monomer identified in Table 17. In some aspects, the one or more monomer weight scores correspond to at least one site monomer identified in Table 18. In some aspects, the method further comprises training the at least one supervised machine learning model using training data, wherein the training data comprises a plurality of peptide structure profiles for a plurality of subjects and a plurality of subject diagnoses for the plurality of subjects. In some aspects, the plurality of subject diagnoses is selected from the group consisting of a positive diagnosis for any subject of the plurality of subjects determined to have a melanoma disease state, a negative diagnosis for any subject of the plurality of subjects determined not to have a melanoma disease state, a positive diagnosis for any subject of the plurality of subjects determined to be likely to benefit from immune checkpoint inhibitory therapy, and a negative diagnosis for any subject of the plurality of subjects determined to be unlikely to benefit from immune checkpoint inhibitory therapy. In some aspects, the plurality of subjects are separated into classes of positive and negative diagnoses using a concordance index as a cutoff between positive and negative diagnoses. In some aspects, the method further comprises performing a differential expression analysis using the training data to compare a first portion of the plurality of subjects with the positive diagnosis for melanoma disease state or subjects unlikely to benefit from immune checkpoint inhibitory therapy, versus a second portion of the plurality of subjects having the negative diagnosis for melanoma disease state or subjects likely to benefit from immune checkpoint inhibitory therapy; and identifying a training group of peptide structures based on the differential expression analysis for use as prognostic markers for the melanoma disease state and/or responsiveness to immune checkpoint inhibitory therapy; and forming the training data based on the training group of peptide structures identified. In some aspects, the at least one supervised machine learning model comprises a logistic regression model, and wherein the at least one supervised learning model compares the negative diagnosis versus the positive diagnosis, wherein the comparison can be at least one non-melanoma state vs at least one melanoma state, or the comparison can be at least one positive response to immune checkpoint inhibitory therapy vs at least one negative response to immune checkpoint inhibitory therapy.

Disclosed herein, in some aspects, is a method of treating melanoma in a subject, the method comprising: A) analyzing one or more monomer weight scores corresponding to at least one site monomer identified in Table 16 using a machine learning model to generate a diagnosis output that classifies the biological sample as evidencing a state associated with melanoma progression, and B) administering a therapeutically effective amount of a treatment for melanoma. In some aspects, the method further comprises receiving a set of raw abundance values of the set of peptide structures and normalizing the set of raw abundance values to a corresponding reference run to generate the set of adjusted-raw abundance values. In some aspects, the method further comprises calculating a site occupancy score, for a given peptide structure at the linking site, as the function of the adjusted-raw abundance value for the given peptide structure and the sum of the set of adjusted-raw abundance values. In some aspects, the method further comprises calculating a site occupancy score, for a given peptide structure at the linking site, as the quotient of the adjusted-raw abundance value for the given peptide structure over the sum of the set of adjusted-raw abundance values. In some aspects, the method further comprises calculating a peptide structure monomer weight score as a function of the site occupancy score and the number of specific monomers for the given peptide structure. In some aspects, the method further comprises calculating a peptide structure monomer weight score as a product of the site occupancy score and the number of specific monomers for the given peptide structure. In some aspects, the method further comprises calculating the a monomer weight score of the one or more monomer weight scores as a sum of peptide structure monomer weight scores for each peptide structure at the linking site. In some aspects, the set of peptide structures comprises post translationally modified (PTM) peptides and/or non-PTM peptides. In some aspects, the monomer is selected from the group consisting of hexose, HexNac, fucose, and sialic acid. In some aspects, the monomer is selected from the group consisting of glucose, mannose, galactose, GlcNAc, GalNAc, fucose, NeuGc, and NeuAc. In some aspects, the set of peptides structures comprises glycosylated peptides and non-glycosylated peptides. In some aspects, the biological sample comprises serum or plasma samples. In some aspects, the reference run comprises serum or plasma samples. In some aspects, the method further comprises treating the biological sample to form a prepared sample comprising the set of peptide structures, the set of peptide structures comprising a set of post translationally modified (PTM) peptides and/or non-PTM peptides; detecting a set of product ions associated with each structure of the set of post translationally modified (PTM) peptides and/or non-PTM peptides, and generating the set of raw abundance values for the set of product ions. In some aspects, the analyzing further comprises: correlating the one or more monomer weight scores with a melanoma disease state to determine a hazard ratio for the melanoma disease state, wherein the hazard ratio is used to update a risk profile of the subject for the melanoma disease state. In some aspects, the method further comprises generating a diagnosis output based on a disease indicator that classifies the biological sample as evidencing a state associated with melanoma progression, wherein the diagnosis output is one of a predictive probability or a risk score. In some aspects, the set of raw abundance values is generated using multiple reaction monitoring mass spectrometry (MRM-MS). In some aspects, the treatment comprises at least one of radiation therapy, chemoradiotherapy, immunotherapy, surgery, hormone therapy, or a targeted drug therapy. In some aspects, the treatment comprises immunotherapy, wherein the immunotherapy is immune checkpoint blockade therapy. In some aspects, the immune checkpoint blockade therapy comprises ipilimumab, nivolumab, and/or pembrolizumab. In some aspects, generating the diagnosis output comprises: generating a report identifying that the biological sample evidences the indication or disease state. In some aspects, the one or more monomer weight scores correspond to at least one site monomer identified in Table 17. In some aspects, the one or more monomer weight scores correspond to at least one site monomer identified in Table 18. In some aspects, the method further comprises training the at least one supervised machine learning model using training data, wherein the training data comprises a plurality of peptide structure profiles for a plurality of subjects and a plurality of subject diagnoses for the plurality of subjects. In some aspects, the plurality of subject diagnoses is selected from the group consisting of a positive diagnosis for any subject of the plurality of subjects determined to have a melanoma disease state, a negative diagnosis for any subject of the plurality of subjects determined not to have a melanoma disease state, a positive diagnosis for any subject of the plurality of subjects determined to be likely to benefit from immune checkpoint inhibitory therapy, and a negative diagnosis for any subject of the plurality of subjects determined to be unlikely to benefit from immune checkpoint inhibitory therapy. In some aspects, the plurality of subjects are separated into classes of positive and negative diagnoses using a concordance index as a cutoff between positive and negative diagnoses. In some aspects, the method further comprises performing a differential expression analysis using the training data to compare a first portion of the plurality of subjects with the positive diagnosis for melanoma disease state or subjects unlikely to benefit from immune checkpoint inhibitory therapy, versus a second portion of the plurality of subjects having the negative diagnosis for melanoma disease state or subjects likely to benefit from immune checkpoint inhibitory therapy; and identifying a training group of peptide structures based on the differential expression analysis for use as prognostic markers for the melanoma disease state and/or responsiveness to immune checkpoint inhibitory therapy; and forming the training data based on the training group of peptide structures identified. In some aspects, the at least one supervised machine learning model comprises a logistic regression model, and wherein the at least one supervised learning model compares the negative diagnosis versus the positive diagnosis, wherein the comparison can be at least one non-melanoma state vs at least one melanoma state, or the comparison can be at least one positive response to immune checkpoint inhibitory therapy vs at least one negative response to immune checkpoint inhibitory therapy. Also disclosed is a system comprising one or more data processors; and a non-transitory computer readable storage medium containing instructions which, when executed on the one or more data processors, cause the one or more data processors to perform part or all of a method disclosed herein. Further disclosed is a computer-program product tangibly embodied in a non-transitory machine-readable storage medium, including instructions configured to cause one or more data processors to perform part or all of a method disclosed herein.

Disclosed herein, in some aspects, is a method of monitoring a subject for a melanoma, the method comprising: receiving first monomer weight score data for a first biological sample obtained from a subject at a first timepoint; analyzing the first monomer weight score data using at least one supervised machine learning model to generate a first disease indicator based on at least one site monomer selected from a group of site monomers identified in Table 16, wherein the group of site monomers in Table 16 comprises a group of site monomers having monomer weight scores associated with melanoma; receiving second monomer weight score data of a second biological sample obtained from the subject at a second timepoint; analyzing the second monomer weight score data using the at least one supervised machine learning model to generate a second disease indicator based on the at least one site monomer selected from the group of site monomers identified in Table 16; and generating a diagnosis output based on the first disease indicator and the second disease indicator. In some aspects, generating the diagnosis output comprises: comparing the second disease indicator to the first disease indicator. In some aspects, the first disease indicator indicates that the first biological sample evidences a negative diagnosis for melanoma and the second biological sample evidences a positive diagnosis for melanoma. In some aspects, the first disease indicator indicates that the first biological sample evidences a melanoma that is not responsive to immunotherapy and the second biological sample evidences a melanoma that is responsive to immunotherapy. In some aspects, the at least one supervised machine learning model comprises a logistic regression model, and wherein the at least one supervised learning model compares negative diagnoses versus positive diagnoses, wherein the comparison can be at least one healthy state versus melanoma generally, healthy state versus immunotherapy responsive melanoma, or immunotherapy nonresponsive melanoma versus immunotherapy responsive melanoma. In some aspects, the at least one site monomer comprises at least one site monomer identified in Table 18. In some aspects, the at least one site monomer comprises at all site monomers identified in Table 18.

Disclosed herein, in some aspects, is a method of treating melanoma in a subject, the method comprising: determining a monomer weight score for at least one site monomer identified in Table 16 in a biological sample from the subject using a multiple reaction monitoring mass spectrometry (MRM-MS) system; analyzing the monomer weight score using at least one machine learning model to generate a disease indicator; generating a diagnosis output based on the disease indicator that classifies the biological sample as evidencing that the patient has melanoma; and administering to the subject a therapeutically effective amount of a melanoma therapy.

Disclosed herein, in some aspects, is a method of treating melanoma in a subject, the method comprising: determining a monomer weight score for at least one site monomer identified in Table 16 in a biological sample from the subject using a multiple reaction monitoring mass spectrometry (MRM-MS) system; analyzing the monomer weight score using at least one machine learning model to generate a disease indicator; generating a diagnosis output based on the disease indicator that classifies the biological sample as evidencing that the melanoma is sensitive to immunotherapy; and administering to the subject a therapeutically effective amount of immunotherapy.

Disclosed herein, in some aspects, is a method of treating melanoma in a subject, the method comprising: determining a monomer weight score for at least one site monomer identified in Table 16 in a biological sample from the subject using a multiple reaction monitoring mass spectrometry (MRM-MS) system; analyzing the monomer weight score using at least one machine learning model to generate a disease indicator; generating a diagnosis output based on the disease indicator that classifies the biological sample as evidencing that the melanoma is sensitive to immunotherapy; and administering to the subject a therapeutically effective amount of immunotherapy.

Disclosed herein, in some aspects, is a method of predicting a risk for melanoma in a subject, the method comprising: determining a monomer weight score for at least one site monomer identified in Table 16 in a biological sample from the subject using a multiple reaction monitoring mass spectrometry (MRM-MS) system; analyzing the monomer weight score using at least one machine learning model to generate a disease indicator; and generating a diagnosis output based on the disease indicator that classifies the biological sample as evidencing that the patient has a risk for melanoma.

Disclosed herein, in some aspects, is a method of predicting immunotherapy sensitivity, the method comprising: determining a monomer weight score for at least one site monomer identified in Table 16 in a biological sample from the subject using a multiple reaction monitoring mass spectrometry (MRM-MS) system; analyzing the monomer weight score using at least one machine learning model to generate a disease indicator; and generating a diagnosis output based on the disease indicator that classifies the biological sample as evidencing that the patient has a risk for melanoma.

In one aspect, a system is described according to various embodiments. In various embodiments, the system comprises one or more data processors and a non-transitory computer readable storage medium containing instructions which, when executed on the one or more data processors, cause the one or more data processors to perform part or all of any one or more of the methods described herein.

In one aspect, disclosed is a computer-program product tangibly embodied in a non-transitory machine-readable storage medium, including instructions configured to cause one or more data processors to perform part or all of any one or more of the methods described herein.

### Section 8—Predicting Peptide Retention Time in Mass Spectrometry

In some embodiments, methods for predicting retention times of peptides include: accessing a feature set corresponding to a peptide, wherein the feature set represents peptide sequence data of the peptide and corresponding physicochemical features; sending the feature set as an input into a neural network, the neural network comprising: (1) a plurality of 1DCNN layers, (2) one or more BiLSTM layers, and (3) a multi-head attention layer; and obtaining, as an output from the neural network, a predicted retention time for the peptide corresponding to an estimated retention time for the peptide in a liquid chromatography mass spectrometry (LC-MS) run. Systems and media may be configured to perform the disclosed methods.

In some embodiments, the neural network may further comprise a flatten and dense layer as a final output layer. In some embodiments, the feature set for a peptide is generated by: encoding a peptide sequence of the peptide to generate a matrix representation of the peptide; compressing the matrix representation to a vector representation; and concatenating, to the vector representation, one or more corresponding physiochemical features that are determined to be associated with the peptide or peptide sequence. In some embodiments, generating the feature set further comprises normalizing the concatenated vector representation between 0 and 1.

In some embodiments, the peptide sequence data is encoded using one-hot encoding. In these embodiments, the matrix representation may comprise: 20 columns corresponding to 20 unique amino acids, and n rows, wherein each row corresponds to a position in a sequence of the corresponding peptide, and wherein n corresponds to a length of the corresponding peptide.

In some embodiments, the peptide sequence data is encoded using BLOSUM 62. In these embodiments, the encoding may generate a matrix comprising: 20 columns corresponding to 20 unique amino acids; 3 columns corresponding to 3 special amino acid characters; 1 column corresponding to a translation stop.

In some embodiments, methods for training a neural network for predicting retention times of peptides include: accessing a plurality of feature sets corresponding to a plurality of peptides, wherein the feature set represents peptide sequence data of the peptide and corresponding physicochemical features; creating a training set comprising a subset of feature sets from the plurality of feature sets; and training a neural network using the training set, the neural network comprising: (1) a plurality of 1DCNN layers, (2) one or more BiLSTM layers, and (3) a multi-head attention layer. Systems and media may be configured to perform the disclosed methods.

In some embodiments, the training may further include creating a validation set comprising a subset of feature sets from the plurality of feature sets; sending the validation set through the neural network; and evaluating the outputs. In some embodiments, the training set comprises 80% of the plurality of feature sets and the validation set comprises 30% of the plurality of feature sets.

### BRIEF DESCRIPTION OF THE DRAWINGS

FIGS. 1A-1C show schematics describing exemplary mass spectrometry-related workflows. FIG. 1A shows a schematic of an example mass spectrometry workflow, from sample collection to data analysis, for glycoproteins. FIG. 1B shows a schematic of certain proteolytic digestion method steps, including denaturation, reduction, alkylation, and proteolytic digestion. FIG. 1C shows a schematic of an example analysis system, including aspects directed to quantification, quality control, and peak integration and data normalization.

FIGS. 2A and 2B show Coomassie stained gel of sample digested with different proteolytic techniques.

FIGS. 3A and 3B show schematics of liquid chromatography systems for sample loading and diversion to waste (FIG. 3A) and sample elution to the mass spectrometer (MS; FIG. 3B).

FIG. 4 shows peak area plots for two glycopeptides as measured (i) without a desalting step and with a chromatographic diversion step, and (ii) with a desalting step.

FIG. 5 shows a plot of the measured false discovery rate of various glycopeptides both (i) without a desalting step and with a chromatographic diversion step, and (ii) with a desalting step.

FIG. 6 shows a plot of peak areas of a species of a glycopeptide measured from sample digestions performed using different amounts of trypsin.

FIGS. 7A and 7B show unity plots comparing various lots and protease configurations for serum samples.

FIGS. 8A and 8B show unity plots comparing various lots and protease configurations for plasma samples.

FIGS. 9A and 9B show unity plots comparing reduction techniques.

FIG. 10 shows a plot of signal response relative to protease quenching time using formic acid.

FIG. 11A shows a plot of CV % of detected peak area from analyses of peptides and glycopeptides performed using specified techniques and sample loading amounts. FIGS. 11B and 11C show plots of log₂ difference for sialyated glycopeptide species having the specified number of terminal sialic acid moieties as assessed for specified sample loading amounts.

FIGS. 12A and 12B show plots of CV % for a control (C) workflow and workflows 1-7 for non-glycosylated peptides (FIG. 12A) and glycopeptides (FIG. 12B). FIGS. 12C and 12D show unity plots comparing various workflows.

FIGS. 13A and 13B show plots of log₂ difference for sialyated glycopeptide species having the specified number of terminal sialic acid moieties as assessed via an AssayMap C18 clean-up taught herein using a 60 μg sample loading amount (FIG. 13A) and an AssayMap RP-S sample clean-up taught herein using a 60 μg sample loading amount (FIG. 13B).

FIG. 14A shows a schematic of an absorbent or bibulous member, such as a blood spot card 1400. FIG. 14B shows a schematic of an absorbent or bibulous member comprising a lateral flow element 1450.

FIG. 15A shows the correlation comparison of peptide abundance for venipuncture serum (HuSer) and finger-prick capillary serum processed from capillary blood (HuCSer). FIG. 15B shows the associated CV values for this same data set.

FIG. 16A shows the correlation comparison of peptide abundance for finger-prick capillary serum (HuCSer) and serum separated from finger-prick blood on Hema Spot membrane (HEMA) and FIG. 16B shows the correlation comparison of peptide abundance for venipuncture serum (HuSer) and serum dried on a dried blood spot card (DSS). FIG. 16C shows the associated CV values HEMA and DSS from this same data set.

FIG. 17A shows the CV comparison for DBS extracted samples and capillary serum processed samples of the clinical trial patient samples. FIGS. 17B and 17C show the correlation of peptide abundance for DBS extracted samples and serum processed samples for a benign pelvic tumor subject (#11) and malignant tumor subject (#26).

FIG. 17D shows PCA clustering of DBS and serum results, wherein each analysis demonstrates the ability to discriminate between benign samples and malignant samples.

FIG. 18 shows the correlation between serum and DBS of glycopeptides and peptides.

FIG. 19 shows a workflow schematic of certain aspects of mass spectrometry-based methodology relevant to the methods taught herein.

FIG. 20 shows a workflow schematic of certain aspects of mass spectrometry-based methodology relevant to the methods taught herein.

FIG. 21 shows a plot of coefficient of variation (CV) from an LC-MS analysis of a 1ss and 2ss serum sample.

FIG. 22 shows a plot of the ratio signal from glycopeptides (AUC) over peptides identified from the same proteins as the identified glycopeptides (AUC) from an LC-MS analysis of a 1ss and 2ss serum sample

FIG. 23 shows a plot of coefficient of variation (CV) from an LC-MS analysis of samples obtained using 80% ACN and 70% ACN HILIC load conditions.

FIG. 24 shows a plot of peak area measurements for a glycopeptide (ATL3_1330_5402-366.1000+) from replicates without HILIC enrichment and a HILIC processing technique taught herein.

FIG. 25 shows a plot of peak area measurements for an unglycosylated peptide (TGLQEVENVK) from replicates without HILIC enrichment and a HILIC processing technique taught herein.

FIG. 26 shows a workflow schematic of certain aspects of mass spectrometry-based methodology relevant to the methods taught herein.

FIG. 27 shows an exemplary workflow for defibrination treatment of plasma samples.

FIG. 28 shows fibrinogen concentration of Na-citrated plasma samples that have been treated with defibrination reagents quantified via a human fibrinogen ELISA assay.

FIG. 29 shows fibrinogen concentration of a variety of different plasma type samples that have been treated with defibrination reagents quantified via a human fibrinogen ELISA assay.

FIG. 30 shows the average relative abundance quantified via LC-MS of A, B, and G fibrinogen peptides for different Na-citrated plasma samples that have been treated with defibrination reagents.

FIGS. 31A and 31B shows a correlation plot of log 2 (abundance) of peptide structures quantified via LC-MS between C-T-K treated defibrinated plasma vs. each of mock treated serum (FIG. 31A) and mock treated plasma (FIG. 31B).

FIG. 32 shows a correlation plot of log₂ (abundance) of peptide structures quantified via LC-MS between C-K treated defibrinated plasma vs. mock treated serum.

FIG. 33 shows a correlation plot of log₂ (abundance) of peptide structures quantified via LC-MS between C treated defibrinated plasma vs. mock treated serum.

FIG. 34 shows a correlation plot of log₂ (abundance) of peptide structures quantified via LC-MS between T treated defibrinated plasma vs. mock treated serum.

FIG. 35 shows fibrinogen concentration of a variety of different plasma type samples that have been treated with defibrination reagents, including silica particles, quantified via a human fibrinogen ELISA assay.

FIG. 36 is a flowchart of a process for analyzing a set of peptide structures in a biological sample in accordance with one or more embodiments.

FIG. 37 is a flowchart of a process for classifying a biological sample with respect to risk of melanoma progression and/or responsiveness to immune checkpoint inhibitor therapy in accordance with one or more embodiments.

FIG. 38 is a flowchart of a process for treating melanoma in a subject in accordance with one or more embodiments.

FIG. 39 is a flowchart of a process for monitoring a subject for melanoma in accordance with one or more embodiments.

FIG. 40 is a schematic example of a process for determining a monomer weight score.

FIG. 41 is a hazard ratio plot showing hazard ratios for each shown site monomer with regards to progression free survival (PFS) in melanoma patients. Filled in diamonds indicate site monomers corresponding to hazard ratios having FDR<0.05.

FIG. 42 is a Kaplan-Meier curve showing progression-free survival of patients in the training cohort characterized as more likely to benefit from immunotherapy or less likely to benefits from immunotherapy, determined based on monomer weight features CFAH_882_fuco and HPT_184_fuco.

FIG. 43 is a Kaplan-Meier curve showing progression-free survival of patients in the validation cohort characterized as more likely to benefit from immunotherapy or less likely to benefits from immunotherapy, determined based on site monomers CFAH_882_fuco and HPT_184_fuco.

FIG. 44 is a Kaplan-Meier curve showing progression-free survival of patients in the test cohort characterized as more likely to benefit from immunotherapy or less likely to benefits from immunotherapy, determined based on site monomers CFAH_882_fuco and HPT_184_fuco.

FIG. 45 is a hazard ratio plot showing hazard ratios for each shown site monomer with regards to progression free survival (PFS) in melanoma patients. Filled in diamonds indicate site monomers corresponding to hazard ratios having FDR<0.05.

FIG. 46A shows Kaplan-Meier curves of various event occurrences in the discovery cohort.

FIG. 46B shows Kaplan-Meier curves of OS and censoring distributions in the discovery and external validation cohorts.

FIG. 47A to 47E show Kaplan-Meier curves stratified by classifier prediction where FIG. 47A-D are for the discovery cohort and FIG. 47E are for the external validation cohort.

FIG. 48A to 48E show that fucosylation signatures in peripheral blood N-glycoproteins are associated with reduced clinical benefit. FIGS. 48A1 and 48A2 are charts of glycopeptides with differential expression, based on relative abundance measurements, in responders compared to non-responders (p<0.05) were classified based on the glycan structure (FIG. 48A1 for fucose and FIG. 48A2 for sialic acid). N-linked glycopeptides separated in two groups based on the presence or absence of fucose that strongly associated with response to treatment (p<0.0001), whereas the number of sialic acid residues did not associate with response. HR, hazard ratio. FIG. 48B shows a chart indicating that di-sialylated O-glycopeptides are enriched in samples with reduced survival (p=0.14). FIG. 48C is a chart showing the effect of site occupancy on protein function in relation to treatment. Lack of a glycan on site N70 of alpha1-antitrypsin (A1AT_N70 NG) is associated with favorable response, whereas absence of glycosylation at the site N1424 of alpha2-microglobulin is associated with poorer responses. The 4-digit number describes glycans composition (number of hexoses, HexNAc, fucose and sialic acid, respectively). FIG. 48D is a chart of hazard ratios of 51 fucose-specific monomer weight features derived from N-glycopeptides sorted by age- and sex-adjusted Cox regression FDR. Hazard ratios of features that achieved FDR<0.05 are filled-in diamonds. FIG. 48E1 to 48E4 show four Kaplan-Meier curves showing performance of repeated five-fold cross-validated LASSO-regularized Cox regression-based classifier using 11 fucose-specific features derived from N-glycopeptides that achieved FDR<0.05 in age- and sex-adjusted Cox regression analysis.

FIG. 49A to 49D show Kaplan-Meier curves of OS in the discovery cohort stratified by melanoma subtype (FIG. 49A), LDH category (FIG. 49B), ECOG performance status (FIG. 49C), and BRAF status (FIG. 49D), respectively.

FIGS. 50A and 50B show Kaplan-Meier curves stratified by early failure (EF, progression and death within 6 months of treatment start, n=40) and sustained controls (SC, progression and death-free beyond 3 years of treatment; n=56) in the discovery cohort. “Other” defines intermediate phenotypes (n=106). FIG. 50A has PFS on the y-axis and FIG. 50B has OS on the y-axis.

FIG. 51A to 51C show Kaplan-Meier curves in the full discovery cohort stratified by classifier prediction and one of LDH category (FIG. 51A), ECOG performance status (FIG. 51B), and BRAF status (FIG. 51C).

FIG. 52 illustrates an example workflow for generating training sets for training a machine learning model for predicting retention times for peptides.

FIG. 53 illustrates the retention time distribution of a particular peptide from serum using the workflow illustrated in FIG. 52.

FIG. 54 illustrates an example LC-MS workflow and data extraction steps that may be employed.

FIG. 55 illustrates an example workflow for predicting retention times based on human serum samples as described herein.

FIG. 56 illustrates a number of different architectures that were attempted for creating a model for predicting peptide retention times.

FIGS. 57A-57B illustrate plots of R2 and R2 Adjusted scores received using the various architectures noted in FIG. 56.

FIG. 58A illustrate an example method for predicting a retention time for a peptide.

FIG. 58B illustrates an example method for training a neural network configured to predict retention times of peptides.

FIG. 59 illustrates an example computer system that may be used to perform one or more steps of one or more methods described or illustrated herein.

FIG. 60 is a block diagram of an analysis system in accordance with one or more embodiments.

FIG. 61 is a block diagram of a computer system in accordance with various embodiments.

### DETAILED DESCRIPTION

Provided herein, in certain aspects, are methods for proteolytically digesting a biological sample comprising a glycoprotein to produce one or more proteolytic glycopeptides, wherein the method comprises use of a thermal denaturation technique. In other aspects, provided herein are methods of performing a liquid chromatography-mass spectrometry (LC-MS) analysis of one or more proteolytic glycopeptides derived from a sample comprising a glycoprotein, including using the proteolytic digestion techniques described herein, wherein the LC-MS technique comprises use of a buffer salt or salt diversion step to eliminate the need for any other online or offline desalting steps. A buffer salt is a salt that is generally resistant to pH change whereas a salt can more generally be any charged ionic species that can potentially contaminate a MS. The disclosure of the present application is based on the inventors' unique perspective and unexpected findings regarding proteolytic digestion techniques and LC-MS techniques providing an improved analysis of glycoproteins and glycopeptides. Specifically, as taught herein, it was unexpectedly found that the use of a thermal denaturation technique enabled more complete digestion of a sample containing glycoproteins. Such thermal denaturation techniques can be performed with control of the temperature of a sample container lid to reduce sample loss via condensation, thereby allowing for improved quantification accuracy and reproducibility. The thermal denaturation techniques developed by the inventors can be performed in a thermocycler, which improves accuracy, reproducibility, and automation of the methods taught herein. Moreover, the resulting proteolytically digested sample was compatible with downstream LC-MS techniques comprising a buffer salt or salt diversion step. Reversed-phase liquid chromatography techniques are well suited to the hydrophilic-hydrophobic characteristic range of non-glycosylated polypeptides, and find use in sample clean-up steps and in chromatography to separate polypeptide species introduced to a mass spectrometer. Typically, polypeptide species have sufficient hydrophobicity to bind to sample phase extraction material based on C18 allowing for a simple desalting step. However, in some embodiments, the glycan structure of a glycopeptide can dramatically adjust the overall behavior of a glycopeptide on a reversed-phase material (e.g., C18) as compared to the non-glycosylated version of the glycopeptide. For example, glycopeptides comprising one or more sialic acid moieties have an increased hydrophilic characteristic and are often lost in conventional desalting techniques because they do not efficiently bind to reverse phase materials. In addition, the use of surfactants for helping digestion can also contribute to the decomposition of sialic acids. Typically, the surfactant needs to be removed from proteolytic digests with a solid phase extraction material before injection into a LC-MS and the acid eluting conditions cause sialic acid decomposition. Such loss results in decreased accuracy of results and quantification. The LC-MS techniques taught herein eliminate the need to perform independent desalting steps, and instead use a buffer salt or salt diversion step during the LC-MS technique to reduce salts introduced to the mass spectrometer while reducing glycopeptides lost due to sample handling. It is worthwhile to note that relatively higher salt concentration can cause a need to perform more frequent maintenance with a MS system where the salt residue needs to be removed through a cleaning process. This cleaning process reduces the overall sample throughput with a MS system since it will be inoperable during the maintenance process. In summary, the methods taught herein provide surprising improvements in the degree of completion of proteolytic digestion, capture of a broader class of glycopeptides that can then be analyzed by the mass spectrometer, reduced biasing of identified and quantified glycopeptides, and improved reproducibility. Such results represent a significant advancement in the ability to use glycoproteins in the study of human physiology.

Thus, in some aspects, provided herein is a method for performing a liquid chromatography-mass spectrometry analysis of a proteolytic glycopeptide derived from a biological sample comprising a glycoprotein, the method comprising: subjecting the biological sample to a thermal denaturation technique to produce a denatured sample followed by a proteolytic digestion technique to produce a proteolytically digested sample comprising the glycopeptide, wherein the thermal denaturation technique subjects the biological sample to a thermal cycle comprising a thermal treatment of about 60° C. to about 100° C., such as about 90° C. to about 100° C., with a hold time of at least about 1 minute, wherein the lid temperature during the thermal cycle is at least about 2° C. higher than the temperature of the block temperature during the thermal cycle, wherein the proteolytic digestion technique comprises adding an amount of one or more proteolytic enzymes and incubating for a digestion incubation time, and wherein the digestion technique comprises quenching the one or more proteolytic enzymes following the digestion incubation time; introducing the proteolytically digested sample to a liquid chromatography (LC) system of a LC-MS system; and performing a LC separation to introduce the proteolytic glycopeptide to a mass spectrometer (MS) system, wherein the LC separation comprises a period of diversion of an initial eluate comprising a buffer salt or salt, and wherein the LC system comprises a reversed-phase chromatography column.

In other aspects, provided herein is a method for proteolytically digesting a biological sample comprising a glycoprotein to produce a proteolytic glycopeptide, the method comprising: subjecting the biological sample to a thermal denaturation technique to produce a denatured sample, wherein the thermal denaturation technique comprises subjecting the biological sample to a thermal cycle comprising a thermal treatment of about 60° C. to about 100° C. with a hold time of at least about 1 minute, wherein the lid temperature during the thermal cycle is at least about 2° C. higher than the temperature of the block temperature during the thermal cycle; subjecting the denatured sample to a reduction technique to produce a reduced sample, wherein the reduction technique comprises adding an amount of a reducing agent to the denatured sample and incubating for a reducing incubation time; subjecting the reduced sample to an alkylation technique to produce an alkylated sample, wherein the alkylation technique comprises adding an amount of an alkylating agent to the reduced sample and incubating substantially in the dark or in a low light condition for an alkylation incubation time, and wherein the alkylated technique comprises quenching the alkylating agent following the alkylation incubation time; and subjecting the alkylated sample to a proteolytic digestion technique to produce a proteolytically digested sample comprising the proteolytic glycopeptide, wherein the proteolytic digestion technique comprises adding an amount of one or more proteolytic enzymes and incubating for a digestion incubation time, and wherein the proteolytic digestion technique comprises quenching the one or more proteolytic enzymes following the digestion incubation time.

The embodiments described herein recognize that glycoproteomics is an emerging field that can be used in the overall diagnosis and/or treatment of subjects with various types of diseases. Glycoproteomics aims to determine the positions, identities, and quantities of glycans and glycosylated proteins in a given sample (e.g., blood sample, serum sample, cell, tissue, etc.). Protein glycosylation is one of the most common and most complex forms of post-translational protein modification, and can affect protein structure, conformation, and function. For example, glycoproteins may play crucial roles in important biological processes such as cell signaling, host-pathogen interactions, and immune response and disease. Glycoproteins may therefore be important to diagnosing different types of diseases.

Although protein glycosylation provides useful information about cancer and other diseases, analysis of protein glycosylation may be difficult as the glycan typically cannot be traced back to the protein site of origin with currently available methodologies. Glycoprotein analysis can be challenging in general due to several reasons. For example, a single glycan composition in a peptide may contain a large number of isomeric structures because of different glycosidic linkages, branching, and many monosaccharides having the same mass. Further, the presence of multiple glycans that share the same peptide sequence may cause the mass spectrometry (MS) signal to split into various glycoforms, lowering their individual abundances compared to the peptides that are not glycosylated (aglycosylated peptides).

However, to understand various disease conditions and to diagnose and prognose certain diseases, such as melanoma, more accurately, it may be important to perform analysis of glycoproteins and to identify not only the glycan but also the linking site (e.g., the amino acid residue of attachment) within the protein. Thus, there is a need to provide a method for site-specific glycoprotein analysis to obtain detailed information about protein glycosylation patterns that may be able to provide information about a disease state (e.g., a melanoma disease state). This information can be used to distinguish the disease state from other states, diagnose a subject as having or not having the disease state, determine a likelihood that a subject has the disease state, determine the responsiveness of a disease to a particular treatment, or a combination thereof. For example, such analysis may be useful in diagnosing a melanoma disease state for a subject (e.g., a negative diagnosis for the melanoma disease state, a positive diagnosis for the melanoma disease state). Sample collection and analysis can be collected at different time points for comparing melanoma disease states over time for a subject. For example, the negative diagnosis may include a healthy state. An example of the positive diagnosis includes the subject suffering from melanoma. A diagnosis can also assess a malignancy status of a previously identified melanoma. Further, a prognosis can assess whether a melanoma is or is not responsive to (or likely to be responsive to) a particular therapy such as immunotherapy (e.g., immune checkpoint inhibitors such as ipilimumab, nivolumab, and/or pembrolizumab).

Accordingly, the embodiments described herein provide various methods and systems for analyzing proteins in subjects and, in particular, glycoproteins. In one or more embodiments, one or more machine learning models are trained to analyze peptide structure data, monomer weight data, or a combination thereof and generate a disease indicator that provides information relating to one or more diseases. For example, in various embodiments, the peptide structure data comprises quantification metrics (e.g., abundance or concentration data) for peptide structures. A peptide structure may be defined by an aglycosylated peptide sequence (e.g., a peptide or peptide fragment of a larger parent protein) or a glycosylated peptide sequence. A glycosylated peptide sequence (also referred to as a glycopeptide structure) may be a peptide sequence having a glycan structure that is attached to a linking site (e.g., an amino acid residue) of the peptide sequence, which may occur via, for example, a particular atom of the amino acid residue). Non-limiting examples of glycosylated peptides include N-linked glycopeptides and O-linked glycopeptides. In some aspects, the monomer weight data comprises one or more monomer weight scores for one or more linker sites. One or more monomer weight scores may be used to generate a disease indicator.

The embodiments described herein recognize that the abundance of one or more monomer type at one or more particular linker sites may be used to determine the likelihood of that subject evidencing a melanoma disease state. Certain peptide structures and monomer weights that are associated with a melanoma disease state may be more relevant to that disease state than other peptide structures that are also associated with that disease state.

Analyzing the abundance of peptide structures and glycosylated peptide structures in a biological sample, along with the monomer weights obtained from analysis of such peptide structures, may provide a more accurate way in which to distinguish a positive melanoma disease state (e.g., a state including the presence of melanoma) from a negative melanoma disease state (e.g., healthy state, an absence of melanoma, etc.). Additionally or alternatively, the disclosed methods may provide a more accurate way in which to predict the responsiveness of a melanoma to immunotherapy (or other) treatment. This type of analysis may be more conducive to generating accurate diagnoses and/or prognoses as compared to glycoprotein analysis that focuses on analyzing glycoproteins that are too large to be resolved via mass spectrometry. Further, with glycoproteins, there may be too many potential proteoforms to consider. Still further, analysis of peptide structure data in the manner described by the various embodiments herein may be more conducive to generating accurate diagnoses as compared to glycomic analysis that provides little to no information about what proteins and to which amino acid residue sites various glycan structures attach.

Further, the methods, systems, and compositions provided by the embodiments described herein may enable an earlier, more accurate and/or less invasive diagnosis of melanoma in a subject as compared to currently available diagnostic modalities (e.g., biopsies, imaging, biochemical tests) used for determining whether immunotherapy (e.g., immune checkpoint inhibitors such as ipilimumab, nivolumab, and/or pembrolizumab) is indicated.

The description below provides exemplary implementations of the methods and systems described herein for analysis of peptide structures and for research, diagnosis, and/or treatment of melanoma. Various examples implement the methods and systems described herein as a screening tool. Descriptions and examples of various terms, as used herein, are provided in the following section.

### A. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polypeptide” and “protein,” as used herein, may be used interchangeably to refer to a polymer comprising amino acid residues, and are not limited to a minimum length. Such polymers may contain natural or non-natural amino acid residues, or combinations thereof, and include, but are not limited to, peptides, polypeptides, oligopeptides, dimers, trimers, and multimers of amino acid residues. Full-length polypeptides or proteins, and fragments thereof, are encompassed by this definition. The terms also include modified species thereof, e.g., post-translational modifications of one or more residues, for example, methylation, phosphorylation glycosylation, sialylation, or acetylation.

The term “glycoprotein,” as used herein, generally refers to a protein having at least one glycan residue bonded thereto. In some embodiments, a glycopeptide, as used herein, refers to a fragment of a glycoprotein, such as obtained from digestion of the glycoprotein.

The term “glycopeptide” or “glycopolypeptide” as used herein, generally refer to a peptide or polypeptide comprising at least one glycan residue. In various embodiments, glycopeptides comprise carbohydrate moieties (e.g., one or more glycans) covalently attached to a side chain of an amino acid residue.

The term “glycopeptide fragment” or “glycosylated peptide fragment” or “glycopeptide” as used herein, generally refers to a glycosylated peptide (or glycopeptide) having an amino acid sequence that is the same as part (but not all) of the amino acid sequence of the glycosylated protein from which the glycosylated peptide is obtained, e.g., ion fragmentation within a MRM-MS instrument. MRM refers to multiple-reaction-monitoring. Unless specified otherwise, within the specification, “glycopeptide fragments” or “fragments of a glycopeptide” refer to the fragments produced directly by using a mass spectrometer optionally after the glycoprotein has been digested enzymatically to produce the glycopeptides.

The terms “glycan” or “polysaccharide,” as used herein, both generally refer to a carbohydrate residue of a glycoconjugate, such as the carbohydrate portion of a glycopeptide, glycoprotein, glycolipid, or proteoglycan. Glycans can include monosaccharides.

The term “linking site” or “glycosylation site” (or, in some cases, simply “site”) as used herein generally refers to the location where a sugar molecule of a glycan or glycan structure is directly bound (e.g., covalently bound) to an amino acid of a peptide, a polypeptide, or a protein. For example, the linking site may be an amino acid residue and a glycan structure may be linked via an atom of the amino acid residue. Non-limiting examples of types of glycosylation can include N-linked glycosylation, O-linked glycosylation, C-linked glycosylation, S-linked glycosylation, and glycation.

The term “amino acid,” as used herein, generally refers to any organic compound that includes an amino group (e.g., —NH2), a carboxyl group (—COOH), and a side chain group (R) which varies based on a specific amino acid. Amino acids can be linked using peptide bonds.

The term “denaturation,” or grammatical equivalents thereof, as used herein, generally refers to any molecule that loses quaternary structure, tertiary structure, and secondary structure which is present in their native state. Non-limiting examples include proteins or nucleic acids being exposed to an external compound or environmental condition such as acid, base, temperature, pressure, and/or radiation.

The term “reduction,” or grammatical equivalents thereof, as used herein, generally refers to the gain of an electron by a substance. In various embodiments, reduction may be used to break disulfide bonds between two cysteines.

The term “alkylation,” or grammatical equivalents thereof, as used herein, generally refers to the transfer of an alkyl group from one molecule to another. In various embodiments, alkylation is used to react with reduced cysteines to prevent the re-formation of disulfide bonds after reduction has been performed.

The terms “digestion” or “enzymatic digestion,” as used herein, generally refers to a biological process that employs enzymes to break specific amino acid peptide bonds. For example, digesting a peptide includes contacting the peptide with an digesting enzyme, e.g., trypsin to produce fragments of the glycopeptide. In some examples, a protease enzyme is used to digest a glycopeptide. The term “protease” refers to an enzyme that performs proteolysis or breakdown of large peptides into smaller polypeptides or individual amino acids. Examples of a protease include, but are not limited to, one or more of a serine protease, threonine protease, cysteine protease, aspartate protease, glutamic acid protease, metalloprotease, asparagine peptide lyase, and any combinations of the foregoing. Enzymatic digestion may be used in preparation for mass spectrometry using trypsin digestion protocols. Proteins may be digested using other proteases in preparation for mass spectrometry if access is limited to cleavage sites.

As used herein, an “internal standard,” may refer to something that can be contained (e.g., spiked-in) in the same sample as a target glycopeptide analyte undergoing mass spectrometry analysis. Internal standards can be used for calibration purposes. Additionally, internal standards can be used in the systems and method described herein. In some aspects, an internal standard can be selected based on similarity m/z and or retention times and can be a “surrogate” if a specific standard is too costly or unavailable. Internal standards can be heavy labeled or non-heavy labeled.

The term “liquid chromatography,” as used herein, generally refers to a technique used to separate a sample into parts, such as spatial separate along a chromatography column. Liquid chromatography can be used to separate, identify, and quantify components.

The term “mass spectrometry,” as used herein, generally refers to an analytical technique used to identify molecules. In various embodiments described herein, mass spectrometry can be involved in characterization and sequencing of proteins.

The term “m/z” or “mass-to-charge ratio” as used herein, generally refers to an output value from a mass spectrometry instrument. In various embodiments, m/z can represent a relationship between the mass of a given ion and the number of elementary charges that it carries. The “m” in m/z stands for mass and the “z” stands for charge. In some embodiments, m/z can be displayed on an x-axis of a mass spectrum.

As used herein, a “transition,” may refer to or identify a peptide structure. In some embodiments, a transition can refer to the specific pair of m/z values associated with a precursor ion and a product or fragment ion.

The terms “biological sample,” as used herein, generally refers to a specimen taken by sampling so as to be representative of the source of the specimen, typically, from a subject. A biological sample can be representative of an organism as a whole, specific tissue, cell type, or category or sub-category of interest. In some embodiments, the biological sample comprises a glycopolypeptide, such as a glycoprotein.

The terms “biological sample,” “biological specimen,” or “biospecimen” as used herein, generally refers to a specimen taken by sampling so as to be representative of the source of the specimen, typically, from a subject. A biological sample can be representative of an organism as a whole, specific tissue, cell type, or category or sub-category of interest. Biological samples may include, but are not limited to stool, synovial fluid, whole blood, blood serum, blood plasma, urine, sputum, tissue, saliva, tears, spinal fluid, tissue section(s) obtained by biopsy; cell(s) that are placed in or adapted to tissue culture; sweat, mucous, gastric fluid, abdominal fluid, amniotic fluid, cyst fluid, peritoneal fluid, pancreatic juice, breast milk, lung lavage, marrow, gastric acid, bile, semen, pus, aqueous humor, transudate, and the like including derivatives, portions and combinations of the foregoing. In some examples, biological samples include, but are not limited, to stool, biopsy, blood and/or plasma. In some examples, biological samples include, but are not limited, to urine or stool. Biological samples include, but are not limited, to biopsy. Biological samples include, but are not limited, to tissue dissections and tissue biopsies. Biological samples include, but are not limited, any derivative or fraction of the aforementioned biological samples. The biological sample can include a macromolecule. The biological sample can include a small molecule. The biological sample can include a virus. The biological sample can include a cell or derivative of a cell. The biological sample can include an organelle. The biological sample can include a cell nucleus. The biological sample can include a rare cell from a population of cells. The biological sample can include any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological sample can include a constituent of a cell. The biological sample can include nucleotides (e.g., ssDNA, dsDNA, RNA), organelles, amino acids, peptides, proteins, carbohydrates, glycoproteins, or any combination thereof. The biological sample can include a matrix (e.g., a gel or polymer matrix) comprising a cell or one or more constituents from a cell (e.g., cell bead), such as DNA, RNA, organelles, proteins, or any combination thereof, from the cell. The biological sample may be obtained from a tissue of a subject. The biological sample can include a hardened cell. Such hardened cells may or may not include a cell wall or cell membrane. The biological sample can include one or more constituents of a cell but may not include other constituents of the cell. An example of such constituents may include a nucleus or an organelle. The biological sample may include a live cell. The live cell can be capable of being cultured.

The term “blood sample,” as used herein, generally refer to a whole blood specimen taken from an individual. In some embodiments, the absorbent or bibulous member may separate components of the blood sample, such as to produce a serum sample or a plasma sample, wherein such produced samples may be referred to herein as a portion of the blood sample. In some embodiments, the blood sample comprises a glycopolypeptide, such as a glycoprotein.

The term “biomarker,” as used herein, generally refers to any measurable substance taken as a sample from a subject whose presence is indicative of some phenomenon. Non-limiting examples of such phenomenon can include a disease state, a condition, or exposure to a compound or environmental condition. In various embodiments described herein, biomarkers may be used for diagnostic purposes (e.g., to diagnose a disease state, a health state, an asymptomatic state, a symptomatic state, etc.). The term “biomarker” may be used interchangeably with the term “marker.”

The term “denatured protein,” as used herein, generally refers to a protein that loses quaternary structure, tertiary structure, and secondary structure which is present in their native state.

The term “peptide,” as used herein, generally refers to amino acids linked by peptide bonds. Peptides can include amino acid chains between 10 and 50 residues. Peptides can include amino acid chains shorter than 10 residues, including, oligopeptides, dipeptides, tripeptides, and tetrapeptides. Peptides can include chains longer than 50 residues and may be referred to as “polypeptides” or “proteins.”

The term “sequence,” as used herein, generally refers to a biological sequence including one-dimensional monomers that can be assembled to generate a polymer. Non-limiting examples of sequences include nucleotide sequences (e.g., ssDNA, dsDNA, and RNA), amino acid sequences (e.g., proteins, peptides, and polypeptides), and carbohydrates (e.g., compounds including Cₘ (H₂O) ₙ).

As used herein, “abundance,” may refer to a quantitative value generated using mass spectrometry. In various embodiments, the quantitative value may relate to an amount of a particular peptide structure (e.g., biomarker) present in a biological sample. In some embodiments, the amount may be in relation to other structures present in the sample (e.g., relative abundance). In some embodiments, the quantitative value may comprise an amount of an ion produced using mass spectrometry. In some embodiments, the quantitative value may be associated with an m/z value (e.g., abundance on x-axis and m/z on y-axis). In other embodiments, the quantitative value may be expressed in atomic mass units.

As used herein, “relative abundance,” may refer to a comparison of two or more abundances. In various embodiments, the comparison may comprise comparing one peptide structure to a total number of peptide structures. In some embodiments, the comparison may comprise comparing one peptide glycoform (e.g., two identical peptides differing by one or more glycans) to a set of peptide glycoforms. In some embodiments, the comparison may comprise comparing a number of ions having a particular m/z ratio by a total number of ions detected. In various embodiments, a relative abundance can be expressed as a ratio. In other embodiments, a relative abundance can be expressed as a percentage. Relative abundance can be presented on a y-axis of a mass spectrum plot.

As used herein, a “subject” or an “individual,” which are terms that are used interchangeably, is a mammal. In some embodiments, a “mammal” includes humans, non-human primates, domestic and farm animals, and zoo, sports, or pet animals, such as dogs, horses, rabbits, cattle, pigs, hamsters, gerbils, mice, ferrets, rats, cats, monkeys, etc. In some embodiments, the subject or individual is human.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For instance, where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure. In some embodiments, two opposing and open ended ranges are provided for a feature, and in such description it is envisioned that combinations of those two ranges are provided herein. For example, in some embodiments, it is described that a feature is greater than about 10 units, and it is described (such as in another sentence) that the feature is less than about 20 units, and thus, the range of about 10 units to about 20 units is described herein.

The term “about” as used herein refers to the usual error range for the respective value readily known in this technical field. Reference to “about” a value or parameter herein includes (and describes) variations that are directed to that value or parameter per se. For example, description referring to “about X” includes description of “X.”

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, including in the appended claims, the singular forms “a,” “or,” and “the” include plural referents unless the context clearly dictates otherwise. For example, “a” or “an” means “at least one” or “one or more.” It is understood that aspects and variations described herein include embodiments “consisting” and/or “consisting essentially of” such aspects and variations.

The use of the term “or” in the claims is used to mean “and/or” unless explicitly indicated to refer to alternatives only or the alternatives are mutually exclusive, although the disclosure supports a definition that refers to only alternatives and “and/or.” For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment. As used herein “another” may mean at least a second or more.

The term “ones” means more than one.

As used herein, the term “plurality” may be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in various embodiments.

“Treating” or treatment of a disease or condition refers to executing a protocol, which may include administering one or more drugs to an individual, such as a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutically effective” as used throughout this application refers to anything that promotes or enhances the well-being of the subject with respect to the medical treatment of this condition. This includes, but is not limited to, a reduction in the frequency or severity of one or more signs or symptoms of a disease, including melanoma.

The term “disease state” as used herein, generally refers to a condition that affects the structure or function of an organism. Non-limiting examples of causes of disease states may include pathogens, immune system dysfunctions, cell damage caused by aging, cell damage caused by other factors (e.g., trauma and cancer). Disease states can include any state of a disease whether symptomatic or asymptomatic. Disease states can include disease stages of a disease progression. Disease states can cause minor, moderate, or severe disruptions in structure or function of an organism (e.g., a subject).

The term “fragment,” as used herein, generally refers to an ion fragmentation process which occurs in a MRM-MS instrument. Fragmenting may produce various fragments having the same mass but varying with respect to their charge, e.g., some biomarkers described herein produce more than one product m/z.

The term “glycopeptide structure monomer weight score,” (also “peptide structure monomer weight score,” used interchangeably) as used herein, generally refers to a value calculated as a function of a site occupancy score of a given peptide structure at a given site and the number of a specific monomer (e.g., specific monosaccharide) for the given glycopeptide structure. In some cases, a glycopeptide structure monomer weight score is a product of the site occupancy score and the number of a specific monomer for the given glycopeptide structure. Thus, as one example, a glycopeptide structure monomer weight score for glycan 5402 at site 33 of the AGP1 protein is the product of the number of a particular type of monomer (e.g., hexose) on that structure and the site occupancy of the 5402 structure at that site. A “glycopeptide structure monomer weight score” may, in some embodiments, be described in terms of a particular type of monomer and/or a particular type of peptide structure. For example a specific glycopeptide structure monomer weight score may be a glycan 5402 hexose weight score, i.e., a glycopeptide structure monomer weight score calculated as a product of the number of hexose molecules on glycan 5402 (i.e., 5) and the site occupancy of glycan 5402 at a particular site.

The term “monomer weight score,” as used herein, generally refers to a value calculated as a sum of individual glycopeptide structure monomer weight scores for all peptide structures at a particular site. In some embodiments, the monomer weight score is a sum of the individual glycopeptide structure monomer weight scores for all peptide structures at a particular site. Thus, as one example, a monomer weight score for hexose at site 33 of the protein AGP1 is a sum of the individual hexose weight scores for each glycan at site 33.

The term “monomer,” as used herein, generally refers to a single or type of unit of a glycan structure. In some aspects, the term “monomer” describes a monosaccharide. Examples of monomers include hexose (e.g., mannose or galactose), HexNac (e.g., GlcNAc or GalNAc), fucose, sialic acid (e.g., NeuAc), mannose, galactose, GlcNAc, and GalNAc.

The term “patient,” as used herein, generally refers to a mammalian subject. The mammal can be a human, or an animal including, but not limited to an equine, porcine, canine, feline, ungulate, and primate animal. In one embodiment, the individual is a human. The methods and uses described herein are useful for both medical and veterinary uses. A “patient” is a human subject unless specified to the contrary.

The term “site monomer,” (also “monomer weight feature”), as used herein, generally refers to a single type of glycan monomer at a particular glycopeptide structure site. Types of glycan monomers include, for example, hexose (i.e., mannose and galactose; referred to herein in some aspect as “hex”), HexNac (i.e., GlcNAc and GalNAc; referred to herein in some aspect as “hexnac”), fucose (i.e., deoxyhexose; referred to herein in some aspect as “fuco”), and sialic acid (i.e., NeuAc; referred to herein in some aspect as “sial”). Thus, as one example, site monomers for site 33 of protein AGP1 include AGP1_33_hex (i.e., a hexose monomer at site 33 of AGP1), AGP1_33_hexnac (i.e., a GlcNAc or GalNAc monomer at site 33 of AGP1), AGP1_33_fuco (i.e., a focuse monomer at site 33 of AGP1), and AGP1_33_sial (i.e., a sialic acid monomer at site 33 of AGP1).

The term “training data,” as used herein generally refers to data that can be input into models, statistical models, algorithms and any system or process able to use existing data to make predictions.

As used herein, a “model” may include one or more algorithms, one or more mathematical techniques, one or more machine learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming. A machine learning algorithm may include a parametric model, a nonparametric model, a deep learning model, a neural network, a linear discriminant analysis model, a quadratic discriminant analysis model, a support vector machine, a random forest algorithm, a nearest neighbor algorithm, a combined discriminant analysis model, a k-means clustering algorithm, a supervised model, an unsupervised model, logistic regression model, a multivariable regression model, a penalized multivariable regression model, or another type of model.

As used herein, an “artificial neural network” or “neural network” (NN) may refer to mathematical algorithms or computational models that mimic an interconnected group of artificial nodes or neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways: when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

As used herein, a “target glycopeptide analyte,” may refer to a peptide structure (e.g., glycosylated or aglycosylated/non-glycosylated), a fraction of a peptide structure, a sub-structure (e.g., a glycan or a glycosylation site) of a peptide structure, a product of one or more of the above listed structures and sub-structures, associated detection molecules (e.g., signal molecule, label, or tag), or an amino acid sequence that can be measured by mass spectrometry.

As used herein, a “peptide data set,” may be used interchangeably with “peptide structure data” and can refer to any data of or relating to a peptide from a resulting mass spectrometry run. A peptide data set can comprise data obtained from a sample or biological sample using mass spectrometry. A peptide dataset can comprise data relating to an external standard, data relating to an internal standard, and data relating to a target glycopeptide analyte of a sample. A peptide data set can result from analysis originating from a single run. In some embodiments, the peptide data set can include raw abundance and mass to charge ratios for one or more peptides.

As used herein, “a transition,” may refer to or identify a peptide structure. In some embodiments, a transition can refer to the specific pair of m/z values associated with a precursor ion and a product or fragment ion.

As used herein, a “non-glycosylated endogenous peptide” (“NGEP”) may refer to a peptide structure that does not comprise a glycan molecule. In various embodiments, an NGEP and a target glycopeptide analyte can originate from the same subject. In various embodiments, an NGEP and a target glycopeptide analyte may be derived from the same protein sequence. In some embodiments, the NGEP and the target glycopeptide analyte may be derived from or include the same peptide sequence. In various embodiments, an NGEP can be labeled with an isotope in preparation for mass spectrometry analysis.

As used herein, “abundance,” may refer to a quantitative value generated using mass spectrometry. In various embodiments, the quantitative value may relate to the amount of a particular peptide structure. In some embodiments, the quantitative value may comprise an amount of an ion produced using mass spectrometry. In some embodiments, the quantitative value may be expressed as an m/z value. In other embodiments, the quantitative value may be expressed in atomic mass units.

As used herein, “relative abundance,” may refer to a comparison of two or more abundances. In various embodiments, the comparison may comprise comparing one peptide structure to a total number of peptide structures. In some embodiments, the comparison may comprise comparing one peptide glycoform (e.g., two identical peptides differing by one or more glycans) to a set of peptide glycoforms. In some embodiments, the comparison may comprise comparing a number of ions having a particular m/z ratio by a total number of ions detected. In various embodiments, a relative abundance can be expressed as a ratio. In other embodiments, a relative abundance can be expressed as a percentage. Relative abundance can be presented on a y-axis of a mass spectrum plot.

As used herein, the term “glycan” refers to the carbohydrate residue of a glycoconjugate, such as the carbohydrate portion of a glycopeptide, glycoprotein, glycolipid, or proteoglycan. Glycans can be monomers or polymers of sugar residues, but typically contain at least three sugars, and can be linear or branched. A glycan may include natural sugar residues (e.g., glucose, N-acetylglucosamine, N-acetylneuraminic acid, galactose, mannose, fucose, hexose, arabinose, ribose, xylose, etc.) and/or modified sugars (e.g., 2′-fluororibose, 2′-deoxyribose, phosphomannose, 6′-sulfo N-acetylglucosamine, etc). The term “glycan” includes homo and heteropolymers of sugar residues. The term encompasses free glycans, including glycans that have been cleaved or otherwise released from a glycoconjugate. Glycan structures (as compared to glycan data formats or representations) are described by a glycan reference code number, and also illustrated in International PCT Patent Application No. PCT/US2020/016286, filed Jan. 31, 2020, which is herein incorporated by reference in its entirety for all purposes.

“Glycomolecule” as used herein includes glycans and glycoconjugates. A glycoconjugate is a molecule that includes a glycan, such as, but not limited to, glycopeptides, glycoproteins, glycolipids, glycoRNA, glycoDNA, etc. Glycomolecule includes fragments of glycoconjugates. As used herein, the term “glycopeptide,” refers to a peptide having at least one glycan residue covalently bonded thereto. A glycopeptide can be an intact protein (e.g., a glycoprotein) or any fragment thereof that has at least one glycan residue covalently bonded thereto.

As used herein, the term “glycoform” refers to a unique primary, secondary, tertiary, and quaternary structure of a protein with an attached glycan of a specific structure.

As used herein, the phrase “glycosylated peptides,” refers to a peptide bonded to a glycan. Glycosylate peptides include peptides that have been covalently modified by glycosylation to become bonded to a glycan.

As used herein, the phrase “glycopeptide fragment” or “glycosylated peptide fragment” or “glycopeptide” refers to a glycosylated peptide (or glycopeptide) having an amino acid sequence that is the same as part (but not all) of the amino acid sequence of the glycosylated protein (or glycoprotein) from which the glycosylated peptide (or glycopeptide) is obtained, e.g., ion fragmentation within a MRM-MS instrument. MRM refers to multiple-reaction-monitoring. Unless specified otherwise, within the specification, “glycopeptide fragments” or “fragments of a glycopeptide” refer to the fragments produced directly by using a mass spectrometer optionally after the glycoprotein has been digested enzymatically to produce the glycopeptides.

As used herein, the phrase “glycoprotein” refers to the glycosylated protein from which the glycosylated peptide is obtained. “Glycoprotein” refers to a protein that contains a peptide backbone covalently linked to one or more sugar moieties (i.e., glycans). As is understood by those skilled in the art, the peptide backbone typically comprises a linear chain of amino acid residues. The sugar moiety(ies) may be in the form of monosaccharides, disaccharides, oligosaccharides, and/or polysaccharides. The sugar moiety(ies) may comprise a single unbranched chain of sugar residues or may comprise one or more branched chains. In certain embodiments, sugar moieties may include sulfate and/or phosphate groups. Alternatively or additionally, sugar moieties may include acetyl, glycolyl, propyl or other alkyl modifications. In certain embodiments, glycoproteins contain O-linked sugar moieties; in certain embodiments, glycoproteins contain N-linked sugar moieties.

As used herein, the phrase “multiple reaction monitoring mass spectrometry (MRM-MS),” refers to a highly sensitive and selective method for the targeted quantification of glycans and peptides in biological samples. Unlike traditional mass spectrometry, MRM-MS is highly selective (targeted), allowing researchers to fine tune an instrument to specifically look for certain peptides' fragments of interest. MRM allows for greater sensitivity, specificity, speed, and quantitation of peptides' fragments of interest, such as a potential biomarker. MRM-MS involves using one or more of a triple quadrupole (QQQ) mass spectrometer and a quadrupole time-of-flight (qTOF) mass spectrometer.

As used herein, the phrase “multiple-reaction-monitoring (MRM) transition,” refers to the mass to charge (m/z) peaks or signals observed when a glycopeptide, or a fragment thereof, is detected by MRM-MS. The MRM transition is detected as the transition of the precursor and product ion.

“Representation” or “format” as used herein with reference to a glycan refers to any linear string of characters intended to convey compositional and/or structural features of a glycan. A glycan representation can be a string that includes symbols and/or alphanumerical characters. In some cases, a glycan representation or format can be constructed using pre-defined rules for representing the compositional and/or structural features of a glycan. For example, a glycan representation in an example format as disclosed herein is N(3)H(3)F(1)A(0). This glycan representation indicates a glycan composed of 3 N-acetyl acetylhexosamine molecules (N), 3 hexose molecules (H), 1 fucose molecule (F), and 0 N-acetylneuraminic acid (A)

As used herein, “platform-specific glycan format” refers to any glycan format that is associated with one or more specific glycomolecule search engines, e.g., one or more specific glycomolecule search engines. A platform-specific glycan format can be used by, or be compatible with, the glycomolecule search engine. In some cases, the platform-specific glycan format is a conventional glycan format used by, or compatible with, conventional glycan databases and/or conventional glycan or glycopeptide search engines. Non-limiting examples of conventional glycan formats include formats used by PGLYCO3, BYONIC and METAMORPHEUS.

“Search engine” refers to any computer-implemented program configured to receive a query (e.g., an input string) and implement algorithms to identify entries within one or more databases that provide a match to the query that meets certain predefined and/or user-specified criteria. A search engine is typically associated with its own proprietary glycan database and can rely on one or more statistical tests to determine the quality of any given match, and provide a confidence score that reflects the quality of a match. A “glycomolecule search engine” refers to any search engine for identifying glycans, glycopeptides, glycolipids, etc., in sample data. Non-limiting examples of glycomolecule or glycopeptide search engines include PGLYCO3, BYONIC and METAMORPHEUS.

Those skilled in the art will recognize that several embodiments are possible within the scope and spirit of the present disclosure. The following description illustrates the disclosure and, of course, should not be construed in any way as limiting the scope of the inventions described herein.

### B. Example Mass Spectrometry and Sample Preparation Workflow

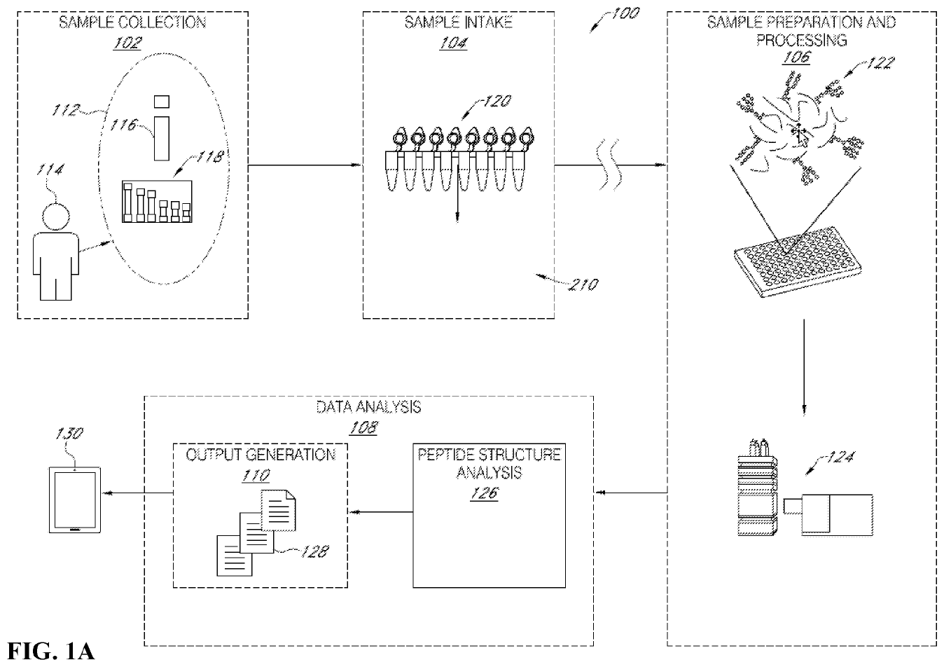

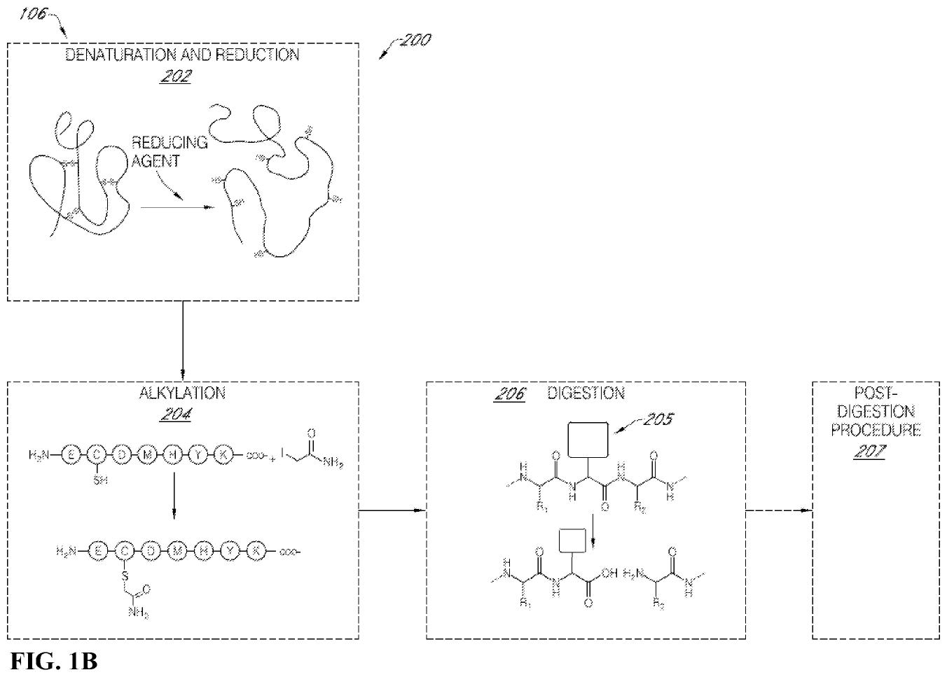

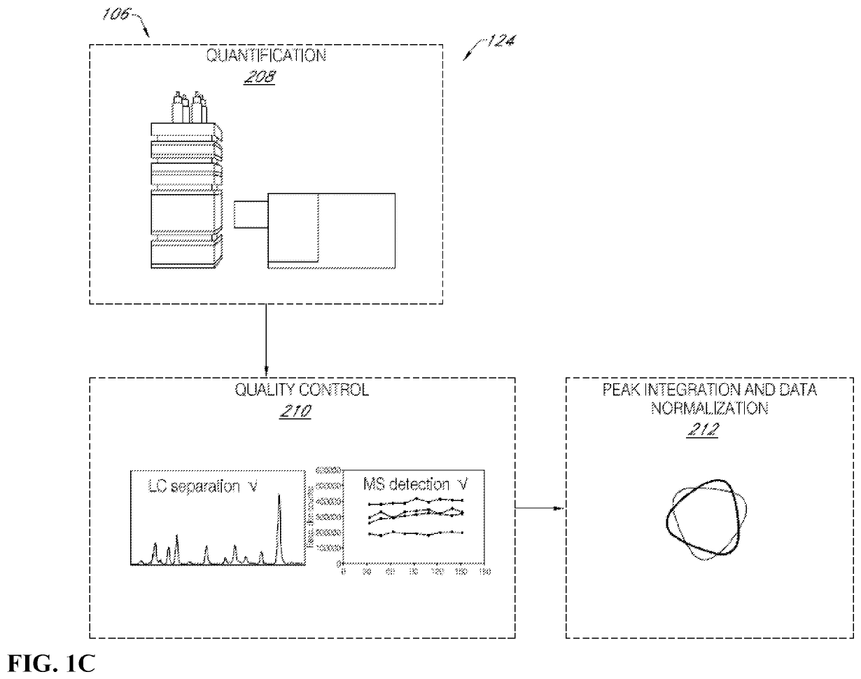

For purposes of orientation and illustration of the description herein, provided in this section are example aspects of sample preparation and mass spectrometry workflows (FIGS. 1A-1C) for analyzing the composition of a peptide and/or glycopeptide using a mass spectrometer. Subsequent sections are provided with more details regarding certain inventive features related to methods for proteolytically digesting a biological sample comprising a glycoprotein, methods of performing a LC-MS analysis of a proteolytic glycopeptide, and mass spectrometry workflows involving any combination of elements thereof.

FIG. 1A is a schematic of an example workflow 100 for a peptide structure analysis, including of glycopeptides. The workflow 100 may include various operations including, for example, sample collection 102, sample intake 104, sample preparation and mass spectrometry processing 106, and data analysis 108.

Sample collection 102 may include, for example, obtaining a biological sample 112 from an individual 114. A biological sample 112 may take the form of a specimen obtained via one or more sampling methods. A biological sample 112 may be representative of an individual 114 as a whole or of a specific tissue, cell type, or other category or sub-category of interest. In some embodiments, the biological sample 112 includes a whole blood sample 116 obtained via a blood draw. In some embodiments, the biological sample 112 includes set of aliquoted samples 118 that include, for example, a serum sample, a plasma sample, a blood cell (e.g., white blood cell (WBC), red blood cell (RBC) sample, another type of sample, or a combination thereof. In some embodiments, the biological sample 112 is a plasma sample from the individual 114. In some embodiments, the biological sample 112 is a serum sample from the individual 114. In some embodiments, the biological sample 112 may include nucleotides (e.g., ssDNA, dsDNA, RNA), organelles, amino acids, peptides, proteins, carbohydrates, glycoproteins, or any combination thereof.

In various embodiments, a single run can analyze a sample (e.g., the sample including a peptide analyte), an external standard (e.g., an NGEP of a serum sample), and an internal standard. As such, abundance or raw abundance for the external standard, the internal standard, and target glycopeptide analyte can be determined by mass spectrometry in the same run.

In various embodiments, external standards may be analyzed prior to analyzing samples. In various embodiments, the external standards can be run independently between the samples. In some embodiments, external standards can be analyzed after every 1, 2, 3, 4, 5, 6, 7, 8, 9, 10, 11, 12, 13, 14, 15, 16, 17, 18, 19, 20, or more experiments. In various embodiments, external standard data can be used in some or all of the normalization systems and methods described herein. In additional embodiments, blank samples may be processed to prevent column fouling.

Sample intake 104 may include one or more various operations such as, for example, aliquoting, labeling, registering, processing, storing, thawing, and/or other types of operations involved with preparing a sample for sample preparation and mass spectrometry processing.

Sample preparation and mass spectrometry processing 106 may include, for example, one or more operations to form set of peptide structures 122, such as a proteolytic peptide and/or a proteolytic glycopeptide. In some embodiments, the sample preparation includes subjecting a biological sample to a proteolytic digestion. Mass spectrometry processing 124 may include, for example, liquid chromatography, introducing species from the sample, and/or derived therefrom, to a mass spectrometer, and data acquisition, such as using a multiple reaction monitoring (MRM) technique. MRM is a mass spectrometry method in which a precursor ion of a particular m/z value, including window thereof, (e.g., peptide analyte) is selected in the first quadrupole (Q1) and transmitted to the second quadrupole (Q2) for fragmentation. The resulting product ions are then transmitted to the third quadrupole (Q3), which detects only product ions with selected predefined m/z values. In some embodiments, the predefined m/z value, including window thereof, selected in the first quadrupole and a predefined m/z value, including window thereof, may be expressed as a MRM transition. Dynamic MRM (dMRM) is a variant of MRM. In dynamic MRM mode, MRM transition lists are scheduled throughout an LC/MS run based on the retention time window for each analyte. In this way, analytes are only monitored while they are eluting from the LC and therefore the MS scan time is not wasted by monitoring the analytes when they are not expected.

Data analysis 108 may include, for example, peptide structure analysis 126, e.g., determining the amino acid sequence of a peptide, determining a site of a post-translational modification, and/or determining a glycan composition and/or structure. In some embodiments, data analysis 108 also includes output generation 110. In some embodiments, output generation 110 may be considered a separate operation from data analysis 108. Output generation 110 may include, for example, generating final output 128 based on the results of peptide structure analysis 126. In some embodiments, the final output 128 may be used for one or more downstream purposes, such as research, diagnosis, and/or treatment, and may be sent to a remote system 130.

In certain aspects, the workflow 100 may optionally exclude one or more of the operations described herein and/or may optionally include one or more other steps or operations other than those described herein (e.g., in addition to and/or instead of those described herein).

FIG. 1B is a schematic of an example workflow 200 for certain sample preparation techniques 106, some of which may be optionally used in methods provided herein. In some embodiments, the workflow 200 comprises a denaturation step 202, such as to unfold and/or linearize a polypeptide to expose one or more cleavage sites. In some embodiments, the workflow 200 comprises a reduction step 202, such as to cleave disulfide bonds. In some embodiments, the workflow 200 comprises an alkylation technique 204, such as to modify cysteine residues to prevent reformation of a disulfide bond. In some embodiments, the workflow 200 comprises a protease digestion technique 206, such as to produce proteolytic peptides, including proteolytic glycopeptides. Box 205 can represent the R group of an amino acid such as, for example, an R group of arginine or lysine that typically will direct a tryptic cleavage. In some embodiments, the workflow 200 may comprise a post-digestion procedure 207, such as any of a desalting technique, addition of a standard, aliquoting, and/or preparation for a mass spectrometry analysis.

FIG. 1C is a schematic of an example workflow for certain mass spectrometry processing techniques 106, some of which may be optionally used in methods provided herein. In some embodiments, the workflow comprises a quantification technique 208 using a mass spectrometer, such as a liquid chromatography-mass spectrometry system. In some embodiments, the workflow comprises a quality control technique 210 configured to optimize data quality. In some embodiments, measures can be put in place allowing only errors within acceptable ranges outside of an expected value. In some embodiments, employing statistical models (e.g., using Westgard rules) can assist in quality control 210. For example, quality control 210 may include, for example, assessing the retention time and abundance of representative peptide structures (e.g., glycosylated and/or aglycosylated) and spiked-in internal standards, in either every sample, or in each quality control sample (e.g., pooled serum digest). In some embodiments, the workflow comprises a peak integration and normalization technique 212 to process the data that has been generated and transform the data into a format for analysis. For example, peak integration and normalization 212 may include converting abundance data for various product ions that were detected for a selected peptide structure into a single quantification metric (e.g., a relative quantity, an adjusted quantity, a normalized quantity, a relative concentration, an adjusted concentration, a normalized concentration, etc.) for that peptide structure. In some embodiments, peak integration and normalization 212 may be performed using one or more of the techniques described in U.S. Patent Publication No. 2020/0372973A1 and/or US Patent Publication No. 2020/0240996A1, the disclosures of which are incorporated by reference herein in their entireties.

### Section 1—Proteolytic Digestion and LC-MS Analysis Techniques for Samples Containing a Glycosylated PolypeptideC. Methods for Proteolytically Digesting a Biological Sample Comprising a Glycoprotein

In certain aspects, provided herein are methods of proteolytically digesting a biological sample comprising a glycoprotein, the methods comprising subjecting the biological sample to a thermal denaturation technique. Proteases are enzymes that cleave polypeptides at, generally, specific cleavage motifs. For example, trypsin is a serine protease that generally cleaves polypeptides at the carboxyl side (C-terminal side) of lysine and arginine residues. A glycan of a glycopeptide may present a steric hindrance to a protease, thereby inhibiting complete protease digestion of a biological sample comprising a glycoprotein. Without being bound to this theory, it is believed that the methods taught herein improve polypeptide unfolding, such as linearization, and provide protease access to cleavage sites thereby providing methods for more complete proteolytic digestion of glycoproteins.

In some aspects, provided is a method comprising subjecting a biological sample to a thermal denaturation technique to produce a denatured sample.

In other aspects, provided is a method comprising subjecting a biological sample to a thermal denaturation technique to produce a denatured sample followed by a proteolytic digestion technique to produce a proteolytically digested sample comprising a proteolytic glycopeptide. In some embodiments, the method comprises quenching one or more proteases used in a proteolytic digestion technique prior to a downstream technique, such as LC-MS.

In other aspects, provided herein is a method comprising: subjecting a biological sample to a thermal denaturation technique to produce a denatured sample; subjecting the denatured sample to a reduction technique to produce a reduced sample; subjecting the reduced sample to an alkylation technique to produce an alkylated sample; and subjecting the alkylated sample to a proteolytic digestion technique to produce a proteolytically digested sample comprising the proteolytic glycopeptide. In some embodiments, the method comprises quenching an alkylating agent used in the alkylation technique prior to subjecting an alkylated sample to a proteolytic digestion technique. In some embodiments, the method comprises quenching one or more proteases used in a proteolytic digestion technique prior to a downstream technique, such as LC-MS.

In some embodiments, the proteolytic digestion methods described herein produce a proteolytic digestion sample, including one comprising a proteolytic glycopeptide, having a digestion completion rate of at least about 70%, such as at least about any of 75%, 80%, 85%, 90%, 91%, 92%, 93%, 94%, 95%, 96%, 97%, 98%, or 99%. In some embodiments, the proteolytic digestion methods described herein produce a proteolytic digestion sample, including one comprising a proteolytic glycopeptide, wherein the sample volume loss is 10% or less, such as 9% or less, 8% or less, 7% or less, 6% or less, 5% or less, 4% or less, 3% or less, 2% or less, or 1% or less, based on all volumes added in producing the proteolytic digestion sample.

In the following sections, additional description of the various aspects of the proteolytic digestion techniques is provided. Such description in a modular fashion is not intended to limit the scope of the disclosure, and based on the teachings provided herein one of ordinary skill in the art will readily appreciate that certain modules can be integrated, at least in part. The section heading used herein are for organizational purposes only and are not to be construed as limiting the subject matter described.

### I. Thermal Denaturation Techniques

In certain aspects, the methods provided herein comprise performing a thermal denaturation technique. Thermal denaturation techniques, generally speaking, change certain polypeptides conformational structures, such as by unfolding and/or linearizing a polypeptide, to enable protease access to cleavage sites. Thermal denaturation techniques described herein comprise subjecting a sample, or a derivative thereof (e.g., a sample diluted with a buffer), to a thermal treatment of about 60° C. to about 100° C. for thermal denaturation incubation time of at least about 1 minute. In some embodiments, the thermal denaturation technique is not performed concurrently with a chemical denaturation technique, such as using high concentrations of denaturing agent, e.g., 6M urea. In some embodiments, the method does not include use of a chemical denaturation technique.

In some embodiments, the thermal denaturation incubation time is performed at a temperature of about 60° C. to about 100° C., such as any of about 70° C. to about 100° C., about 80° C. to about 100° C., about 90° C. to about 100° C., about 95° C. to about 100° C., or about 85° C. to about 95° C. In some embodiments, the thermal denaturation incubation time is performed at a temperature of at least about 60° C., such as at least about any of 65° C., 70° C., 75° C., 80° C., 85° C., 90° C., 95° C., or 100° C. In some embodiments, the thermal denaturation incubation time is performed at a temperature of about 100° C. or less, such as about any of 95° C. or less, 90° C. or less, 85° C. or less, 80° C. or less, 75° C. or less, 70° C. or less, 65° C. or less, 60° C. or less. In some embodiments, the thermal denaturation incubation time is performed at a temperature of about any of 60° C., 65° C., 70° C., 75° C., 80° C., 85° C., 90° C., 91° C., 92° C., 93° C., 94° C., 95° C., 96° C., 97° C., 98° C., 99° C., or 100° C.

In some embodiments, the thermal denaturation incubation time is about 1 minute to about 15 minutes, such as any of about 1 minute to about 5 minutes, about 1 minute to about 10 minutes, about 2.5 minutes to about 7.5 minutes, or about 5 minutes to about 15 minutes. In some embodiments, the thermal denaturation incubation time is at least about 1 minute, such as at least about any of 1.5 minutes, 2 minutes, 2.5 minutes, 3 minutes, 3.5 minutes, 4 minutes, 4.5 minutes, 5 minutes, 5.5 minutes, 6 minutes, 6.5 minutes, 7 minutes, 7.5 minutes, 8 minutes, 8.5 minutes, 9 minutes, 9.5 minutes, 10 minutes, 11 minutes, 12 minutes, 13 minutes, 14 minutes, or 15 minutes. In some embodiments, the thermal denaturation incubation time is about 15 minutes or less, such as about any of 14 minutes or less, 13 minutes or less, 12 minutes or less, 11 minutes or less, 10 minutes or less, 9.5 minutes or less, 9 minutes or less, 8.5 minutes or less, 8 minutes or less, 7.5 minutes or less, 7 minutes or less, 6.5 minutes or less, 6 minutes or less, 5.5 minutes or less, 5 minutes or less, 4.5 minutes or less, 4 minutes or less, 3.5 minutes or less, 3 minutes or less, 2.5 minutes or less, 2 minutes or less, 1.5 minutes or less, or 1 minute or less. In some embodiments, the thermal denaturation incubation time is about any of 1 minute, 1.5 minutes, 2 minutes, 2.5 minutes, 3 minutes, 3.5 minutes, 4 minutes, 4.5 minutes, 5 minutes, 5.5 minutes, 6 minutes, 6.5 minutes, 7 minutes, 7.5 minutes, 8 minutes, 8.5 minutes, 9 minutes, 9.5 minutes, 10 minutes, 11 minutes, 12 minutes, 13 minutes, 14 minutes, or 15 minutes.

In some embodiments, the thermal denaturation technique comprises a thermal denaturation incubation time of about 1 minute to about 15 minutes, such as about any of 1 minute, 2 minutes, 3 minutes, 4 minutes, 5 minutes, 6 minutes, 7 minutes, 8 minutes, 9 minutes 10 minutes, 11 minutes, 12 minutes, 13 minutes, 14 minutes, or 15 minutes, wherein the thermal denaturation incubation is performed at a temperature of about 60° C. to about 100° C., such about any of 60° C., 65° C., 70° C., 75° C., 80° C., 85° C., 90° C., 91° C., 92° C., 93° C., 94° C., 95° C., 96° C., 97° C., 98° C., 99° C., or 100° C.

The thermal denaturation incubation temperature can be controlled by numerous techniques and combinations thereof. In some embodiments, the thermal denaturation incubation temperature is controlled by a water bath. In some embodiments, the thermal denaturation incubation temperature is controlled by a heat block. In some embodiments, the thermal denaturation incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the thermal denaturation incubation time temperature are controlled by a technique described herein, such as cooling at room temperature or via the thermocycler.

In some embodiments, the thermal denaturation technique comprises subjecting a sample, or a derivative thereof, e.g., a sample diluted in a buffer, to a thermal cycle. In some embodiments, the thermal cycle comprises subjecting the sample, or a derivative thereof, to one or more of: (a) a block starting temperature (b) block set temperature (the temperature for the thermal denaturation incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 60° C. to about 100° C., including about any of 60° C., 65° C., 70° C., 75° C., 80° C., 85° C., 90° C., 91° C., 92° C., 93° C., 94° C., 95° C., 96° C., 97° C., 98° C., 99° C., or 100° C. In some embodiments, the thermal cycle comprises: (a) a set block temperature of about 60° C. to about 100° C., including about any of 60° C., 65° C., 70° C., 75° C., 80° C., 85° C., 90° C., 91° C., 92° C., 93° C., 94° C., 95° C., 96° C., 97° C., 98° C., 99° C., or 100° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 50° C., such as any of 15° C. to about 25° C., about 20° C. to about 30° C., or about 20° C. to about 25° C., including about any of 20° C., 21° C., 22° C., 23° C., 24° C., 25° C., (b) a set block temperature of about 60° C. to about 100° C., such about any of 60° C., 65° C., 70° C., 75° C., 80° C., 85° C., 90° C., 91° C., 92° C., 93° C., 94° C., 95° C., 96° C., 97° C., 98° C., 99° C., or 100° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C. In some embodiments, the lid temperature during the thermal cycle may be the same respective temperature of the block during the thermal cycle or a temperature greater than the temperature of the block during the thermal cycle.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

In some embodiments, the method further comprises admixing an amount of a biological sample a buffer prior to the thermal denaturation technique (e.g., the buffered sample is subjected to a thermal denaturation technique described herein). In some embodiment, the amount (as assessed based on the final concentration in the sample containing solution containing solution) of the buffer is about 1 mM to about 100 mM, such as any of about 20 mM to about 80 mM, about 30 mM to about 70 mM, or about 40 mM to about 60 mM. In some embodiment, the amount of the buffer is about any of 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some embodiments, the buffer is selected from the group consisting of ammonium bicarbonate, ammonium acetate, ammonium formate, triethylammonium bicarbonate, and Tris-HCl, or any combination thereof.

In some embodiments, the method further comprises determining the protein concentration in a biological sample or a derivative thereof.

### II. Reduction Techniques

In certain aspects, the methods provided herein comprise performing a reduction technique. In some embodiments, the reduction technique is performed on a sample, or a derivative thereof, following thermal denaturation. Reduction techniques, generally speaking, reduce (e.g., cleave) disulfide linkages between cysteine residues of one or more polypeptides to reduce the presence of polypeptide conformations that inhibit or prevent protease cleavage of the one or more polypeptides. Reduction techniques described herein comprise subjecting a sample, or a derivative thereof (e.g., a denatured sample), to an amount of a reducing agent and incubating for a reducing incubation time performed at a temperature or range thereof. In some embodiments, the reducing agent is dithiothreitol (DTT), tris(2-carboxyethyl) phosphine (TCEP), beta-mercaptoethanol (BME), or a cysteine, or any mixture thereof.

In some embodiments, the amount (as assessed based on the final concentration in the sample containing solution containing solution) of a reducing agent, e.g., DTT, used in a reduction technique is about 1 mM to about 100 mM, such as any of about 1 mM to about 40 mM, about 1 mM to about 30 mM, about 5 mM to about 25 mM, about 10 mM, to about 20 mM, 20 mM to about 80 mM, about 30 mM to about 70 mM, or about 40 mM to about 60 mM. In some embodiments, the amount of reducing agent used in a reduction technique is at least about 1 mM, such as at least about any of 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some embodiments, the amount of reducing agent used in a reduction technique is about 100 mM or less, such as about any of 95 mM or less, 90 mM or less, 85 mM or less, 80 mM or less, 75 mM or less, 70 mM or less, 65 mM or less, 60 mM or less, 55 mM or less, 50 mM or less, 45 mM or less, 40 mM or less, 35 mM or less, 30 mM or less, 25 mM or less, 20 mM or less, 19 or less, 18 or less, 17 or less, 16 or less, 15 or less, 14 or less, 13 or less, 12 or less, 11 or less, 10 or less, 9 or less, 8 or less, 7 or less, 6 or less, 5 or less, 4 or less, 3 or less, 2 or less, or 1 or less. In some embodiments, the amount of reducing agent used in a reduction technique is about any of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In some embodiments, the reduction incubation time is about 10 minutes to about 120 minutes, such as any of about 30 minutes to about 60 minutes, about 40 minutes to about 60 minutes, about 45 minutes to about 55 minutes. In some embodiments, the reduction incubation time is at least about 20 minutes, such as at least about any of 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes. In some embodiments, the reduction incubation time is about 120 minutes or less, such as about any of 115 minutes or less, 110 minutes or less, 105 minutes or less, 100 minutes or less, 95 minutes or less, 90 minutes or less, 85 minutes or less, 80 minutes or less, 75 minutes or less, 70 minutes or less, 65 minutes or less, 60 minutes or less, 55 minutes or less, 50 minutes or less, 45 minutes or less, 40 minutes or less, 35 minutes or less, 30 minutes or less, or 25 minutes or less. In some embodiments, the reduction incubation time is about any of 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes.

In some embodiments, the reduction incubation time is performed at a temperature of about 20° C. to about 100° C., such as any of about 40° C. to about 80° C., about 50° C. to about 70° C., about 50° C. to about 60° C., about 55° C. to about 65° C., or about 60° C. to about 70° C. In some embodiments, the reduction incubation time is performed at a temperature of at least about 20° C., such as at least about any of 25° C., 30° C., 35° C., 40° C., 45° C., 50° C., 55° C., 60° C., 65° C., 70° C., 75° C., 80° C., 85° C., 90° C., or 95° C. In some embodiments, the reduction incubation time is performed at a temperature of about 95° C. or less, such as about any of 90° C. or less, 85° C. or less, 80° C. or less, 75° C. or less, 70° C. or less, 65° C. or less, 60° C. or less, 55° C. or less, 50° C. or less, 45° C. or less, 40° C. or less, 35° C. or less, 30° C. or less, or 25° C. or less. In some embodiments, the reduction incubation time is performed at a temperature of about any of 20° C., 25° C., 30° C., 35° C., 40° C., 45° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 75° C., 80° C., 85° C., 90° C., or 95° C. In some embodiments, the reduction incubation time is performed at a room temperature.

In some embodiments, the reduction technique comprises a reduction incubation time of about 30 minutes to about 70 minutes, such as about any of 35 minutes, 40 minutes, 45 minutes, 50 minutes, 55 minutes, 60 minutes, or 65 minutes, wherein the reduction incubation time is performed at a temperature of about 50° C. to about 70° C., such about any of 55° C., 60° C., or 65° C. In some embodiments, the reduction technique comprises use of an amount (as assessed based on the final concentration in the sample containing solution) of a reducing agent, e.g., DTT, of about 5 mM to about 25 mM, such as any of about 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, or 24 mM, and a reduction incubation time of about 30 minutes to about 70 minutes, such as about any of 35 minutes, 40 minutes, 45 minutes, 50 minutes, 55 minutes, 60 minutes, or 65 minutes, wherein the reduction incubation time is performed at a temperature of about 50° C. to about 70° C., such about any of 55° C., 60° C., or 65° C.

The reduction incubation temperature can be controlled by numerous techniques and combinations thereof. In some embodiments, the reduction incubation temperature is controlled by an ambient temperature, such as room temperature. In some embodiments, the reduction incubation temperature is controlled by a water bath. In some embodiments, the reduction incubation temperature is controlled by a heat block. In some embodiments, the reduction incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the reduction incubation time temperature are controlled by a technique described herein, such as cooling at room temperature or a ramp rate.

In some embodiments, the reduction technique comprises subjecting a sample, or a derivative thereof, e.g., a denatured sample, to a thermal cycle. In some embodiments, the thermal cycle comprises subjecting the sample, or a derivative thereof, to one or more of: (a) a block starting temperature (b) block set temperature (the temperature for the reduction incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 20° C. to about 100° C., such as any of 40° C. to about 80° C., about 50° C. to about 70° C., about 50° C. to about 60° C., about 55° C. to about 65° C., or about 60° C. to about 70° C., including about any of 50° C., 55° C., 60° C., 65° C., or 70° C. In some embodiments, the thermal cycle comprises: (a) a set block temperature of about 20° C. to about 100° C., such as any of about 40° C. to about 80° C., about 50° C. to about 70° C., about 50° C. to about 60° C., about 55° C. to about 65° C., or about 60° C. to about 70° C., including about any of 50° C., 55° C., 60° C., 65° C., or 70° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 60° C., such as any of about 15° C. to about 50° C., about 20° C. to about 40° C., about 20° C. to about 30° C., or about 20° C. to about 25° C., (b) a set block temperature of about 20° C. to about 100° C., such as any of about 40° C. to about 80° C., about 50° C. to about 70° C., about 50° C. to about 60° C., about 55° C. to about 65° C., or about 60° C. to about 70° C., including about any of 50° C., 55° C., 60° C., 65° C., or 70° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

In some embodiments, the reduction technique described herein is completed simultaneously with a thermal denaturation step. For example, the combined thermal denaturation technique and reduction technique comprises adding a reducing agent to a sample, or a derivative thereof, and then subjecting the sample, or the derivative thereof, to a temperature of about 60° C. to about 100° C., such about any of 60° C., 65° C., 70° C., 75° C., 80° C., 85° C., 90° C., 91° C., 92° C., 93° C., 94° C., 95° C., 96° C., 97° C., 98° C., 99° C., or 100° C., for an incubation time of at least about 1 minute, such as at least about any of 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes, or 60 minutes. In some embodiments, the combined thermal denaturation technique and reduction technique comprises adding a reducing agent to a sample, or a derivative thereof, and then subjecting the sample, or the derivative thereof, to a temperature of about 90° C. to about 100° C., for an incubation time of about 40 minutes to about 60 minutes, including 50 minutes.

### III. Alkylation Techniques

In certain aspects, the methods provided herein comprise performing an alkylation technique. In some embodiments, the alkylation technique is performed on a sample, or a derivative thereof, following the performance of a reduction technique. Alkylation techniques, generally speaking, prevent the reformation of one or more disulfide linkages between, e.g., cysteine residues of one or more polypeptides. This is done by, e.g., the addition of an acetamide moiety to the sulfur of a cysteine residue thereby producing an alkylated polypeptide. Alkylation techniques may reduce the presence of polypeptide conformations that inhibit or prevent protease cleavage of the one or more polypeptides. Alkylation techniques described herein comprise subjecting a sample, or a derivative thereof (e.g., a reduced sample), to an amount of an alkylating agent and incubating for an alkylation incubation time performed at a temperature or range thereof. In some embodiments, the method comprises subjecting a denatured sample to a reduction technique followed by an alkylation technique prior to performing a proteolytic digestion technique.

In some embodiments, the alkylating agent is iodoacetamide (IAA), 2-chloroacetamide, an acetamide salt, or any mixture thereof.

In some embodiments, the amount (as assessed based on the final concentration in the sample containing solution) of an alkylating agent, e.g., IAA, used in an alkylation technique is about 10 mM to about 100 mM, such as any of about 10 mM to about 50 mM, about 20 mM to about 40 mM, about 20 mM to about 36 mM, about 15 mM to about 25 mM, about 20 mM to about 25 mM, about 20 mM to about 80 mM, about 30 mM to about 70 mM, or about 40 mM to about 60 mM. In some embodiments, the amount of an alkylating agent used in an alkylation technique is at least about 10 mM, such as at least about any of 15 mM, 16 mM, 17 mM, 18 mM, 19 mM, 20 mM, 21 mM, 22 mM, 23 mM, 24 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some embodiments, the amount of an alkylating agent used in an alkylation technique is about 100 mM or less, such as about any of 95 mM or less, 90 mM or less, 85 mM or less, 80 mM or less, 75 mM or less, 70 mM or less, 65 mM or less, 60 mM or less, 55 mM or less, 50 mM or less, 45 mM or less, 40 mM or less, 35 mM or less, 30 mM or less, 25 mM or less, 24 mM or less, 23 mM or less, 22 mM or less, 21 mM or less, 20 mM or less, 19 mM or less, 18 mM or less, 17 mM or less, 16 mM or less, 15 mM or less, or 10 mM or less. In some embodiments, the amount of an alkylating agent used in an alkylation technique is about any of 10 mM, 15 mM, 20 mM, 20.5 mM, 21 mM, 21.5 mM, 22 mM, 22.5 mM, 23 mM, 23.5 mM, 24 mM, 24.5 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In some embodiments, the alkylation incubation time is about 5 minutes to about 60 minutes, such as any of about 10 minutes to about 50 minutes, about 20 minutes to about 40 minutes, about 25 minutes to about 35 minutes. In some embodiments, the alkylation incubation time is at least about 5 minutes, such as at least about any of 10 minutes, 15 minutes, 20 minutes, 25 minutes, 30 minutes, 35 minutes, 40 minutes, 45 minutes, 50 minutes, 55 minutes, or 60 minutes. In some embodiments, the alkylation incubation time is about 60 minutes or less, such as about any of 55 minutes or less, 50 minutes or less, 45 minutes or less, 40 minutes or less, 35 minutes or less, 30 minutes or less, 25 minutes or less, 20 minutes or less, 15 minutes or less, 10 minutes or less, or 5 minutes or less. In some embodiments, the alkylation incubation time is about any of 5 minutes, 10 minutes, 15 minutes, 20 minutes, 25 minutes, 30 minutes, 35 minutes, 40 minutes, 45 minutes, 50 minutes, 55 minutes, or 60 minutes.

In some embodiments, the alkylation incubation time is performed at a temperature of about 15° C. to about 100° C., such as any of about 15° C. to about 80° C., about 15° C. to about 60° C., about 15° C. to about 35° C., about 20° C. to about 30° C., or about 20° C. to about 25° C. In some embodiments, the alkylation incubation time is performed at a temperature of at least about 15° C., such as at least about any of 20° C., 21° C., 22° C., 23° C., 24° C., 25° C., 30° C., 35° C., 40° C., 45° C., 50° C., 55° C., 60° C., 65° C., 70° C., 75° C., 80° C., 85° C., 90° C., or 95° C. In some embodiments, the alkylation incubation time is performed at a temperature of about 95° C. or less, such as about any of 90° C. or less, 85° C. or less, 80° C. or less, 75° C. or less, 70° C. or less, 65° C. or less, 60° C. or less, 55° C. or less, 50° C. or less, 45° C. or less, 40° C. or less, 35° C. or less, 30° C. or less, 25° C. or less, 24° C. or less, 23° C. or less, 22° C. or less, 21° C. or less, or 20° C. or less. In some embodiments, the alkylation incubation time is performed at a temperature of about any of 15° C., 16° C., 17° C., 18° C., 19° C., 20° C., 21° C., 22° C., 23° C., 24° C., 25° C., 30° C., 35° C., 40° C., 45° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 75° C., 80° C., 85° C., 90° C., or 95° C. In some embodiments, the alkylation incubation time is performed at a room temperature.

In some embodiments, the alkylation technique comprises an alkylation incubation time of about 5 minutes to about 60 minutes, such as about any of 15 minutes, 20 minutes, 25 minutes, 30 minutes, 35 minutes, or 40 minutes, wherein the alkylation incubation time is performed at a temperature of about 15° C. to about 30° C., such about any of 20° C., 21° C., 22° C., 23° C., 24° C., 25° C. In some embodiments, the alkylation technique comprises use of an amount (containing solution based on the final concentration in the sample) of an alkylating agent, e.g., IAA, of about 15 mM to about 40 mM, such as any of about 20 mM, 20.5 mM, 21 mM, 21.5 mM, 22 mM, 22.5 mM, 23 mM, 23.5 mM, 24 mM, 24.5 mM, 25 mM, 25.5 mM, 26 mM, 26.5 mM, 27 mM, 27.5 mM, 28 mM, 28.5 mM, 29 mM, 29.5 mM, 30 mM, 30.5 mM, 31 mM, 31.5 mM, 32 mM, 32.5 mM, 33 mM, 33.5 mM, 34 mM, 34.5 mM, 35 mM, 35.5 mM, 36 mM, 36.5 mM, 37 mM, 37.5 mM, 38 mM, and an alkylation incubation time of about 5 minutes to about 60 minutes, such as about any of 15 minutes, 20 minutes, 25 minutes, 30 minutes, 35 minutes, or 40 minutes, wherein the alkylation incubation time is performed at a temperature of about 15° C. to about 30° C., such about any of 20° C., 21° C., 22° C., 23° C., 24° C., or 25° C.

The alkylation incubation temperature can be controlled by numerous techniques and combinations thereof. In some embodiments, the alkylation incubation temperature is controlled by an ambient temperature, such as room temperature. In some embodiments, the alkylation incubation temperature is controlled by a water bath. In some embodiments, the alkylation incubation temperature is controlled by a heat block. In some embodiments, the alkylation incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the alkylation incubation time temperature are controlled by a technique described herein, such as cooling at room temperature or a ramp rate.

In some embodiments, the alkylation technique comprises subjecting a sample, or a derivative thereof, e.g., a denatured sample, to a thermal cycle. In some embodiments, the thermal cycle comprises subjecting the sample, or a derivative thereof, to one or more of: (a) a block starting temperature (b) block set temperature (the temperature for the alkylation incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., or 25° C. In some embodiments, the thermal cycle comprises: (a) a set block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., 25° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., 25° C., (b) a set block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., 25° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C.

In some embodiments, the alkylation technique further comprises quenching the alkylating agent comprising use of a neutralizing agent. In some embodiments, the neutralizing agent is a reducing agent. In some embodiments, the reducing agent is dithiothreitol (DTT), tris(2-carboxyethyl) phosphine (TCEP), beta-mercaptoethanol (BME), or a cysteine, or any mixture thereof. In some embodiments, the neutralizing agent is added in an amount to fully quench the amount of the alkylating agent, such as in an amount greater than or equal to a molar amount of an active moiety of the alkylating agent. In some embodiments, the amount (as assessed based on the final concentration in the sample containing solution) of the neutralizing agent is about 1 mM to about 100 mM.

In some embodiments, the alkylation technique, in whole or in part, is performed substantially in a low light condition. In some embodiments, the alkylation incubation time is performed in a low light condition. In some embodiments, the low light condition is in the dark or a location substantially devoid of sunlight and/or room lighting, such as in a desk drawer. In some embodiments, the low light condition is a filtered light, such as red light.

In some embodiments, the alkylating agent is sourced from a stock solution. In some embodiments, the stock solution is prepared within about 1 hour, such as within about any of 50 minutes, 40 minutes, 30 minutes, 20 minutes, or 10 minutes, of use.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

### IV. Proteolytic Digestion Techniques

In certain aspects, the methods provided herein comprise performing a proteolytic digestion technique. In some embodiments, the proteolytic digestion technique is performed on a sample, or a derivative thereof, following thermal denaturation and/or any additional steps intended to expose protease cleavage sites. Proteolytic digestion techniques, generally speaking, cleave polypeptides at known cleavage sites. For example, trypsin is a serine protease that generally cleaves polypeptides at the carboxyl side (C-terminal side) of lysine and arginine residues. Certain exceptions apply to the cleavage pattern of trypsin, such as due to proximity of a proline residue and/or a post-translational modification causing steric hindrance relative to the cleavage site. Proteolytic digestion techniques described herein comprise subjecting a sample, or a derivative thereof (e.g., a denatured sample or an alkylated sample, including an alkylated sample subjected to a reduction technique prior to an alkylation technique), to an amount of one or more proteases and incubating for a digestion incubation time performed at a temperature or range thereof.

In some embodiments, each of the one or more proteases is trypsin, LysC, LysN, AspN, GluC, ArgC, IdeS, IdeZ, PNGase F, thermolysin, pepsin, elastase, TEV, or Factor Xa, or any mixture thereof. In some embodiments, wherein two or more proteases are used, the weight ratio between a first protease and a second protease is about 1:10 to about 10:1, such as about any of about 1:9, 1:8, 1:7: 1:6, 1:5, 1:4, 1:3, 1:2, or 1:1. In some embodiments, the one or more proteases is trypsin. In some embodiments, the one or more proteases is a mixture of trypsin and LysC, such as in a weight ratio of about 1:1. In some embodiments, the one or more proteases is selected based on the type and/or characteristic of a biological sample used in the methods herein. In some embodiments, the biological sample is a plasma sample, wherein the one or more proteases is trypsin and Lys-C, such as in a weight ratio of about 1:1. In some embodiments, the biological sample is a serum sample, wherein the one or more proteases is trypsin. In some embodiments, the protease is a modified protease, such as comprising a modification to prevent or inhibit self-proteolysis. In some embodiments, the modified protease is a modified trypsin, such as a methylated and/or an acetylated trypsin. In some embodiments, the modified trypsin is a tosyl phenylalanyl chloromethyl ketone (TPCK)-treated trypsin.

In some embodiments, the amount of a protease, e.g., trypsin or LysC, used in a proteolytic digestion technique is based on a weight ratio relative to the polypeptide content of a sample, or a derivative thereof, (i.e., weight of a protease: weight of polypeptide content) of about 1:200 to about 1:10, such as any of about 1:100 to about 1:10, about 1:50 to about 1:10, about 1:40 to about 1:20, about 1:50 to about 1:30, about 1:45 to about 1:35, about 1:20 to about 1:40, about 1:30 to about 1:10, or about 1:25 to about 1:15. In some embodiments, the amount of a protease used in a proteolytic digestion technique is at least about 1:200, such as at least about any of 1:190, 1:180, 1:170, 1:160, 1:150, 1:140, 1:130, 1:120, 1:110, 1:100, 1:95, 1:90, 1:85, 1:80, 1:75, 1:70, 1:65, 1:60, 1:55, 1:50, 1:45, 1:40, 1:35, 1:30, 1:25, 1:20, 1:15, or 1:10. In some embodiments, the amount of a protease used in a proteolytic digestion technique is about 1:10 or less, such as about any of 1:15 or less, 1:20 or less, 1:25 or less, 1:30 or less, 1:35 or less, 1:40 or less, 1:45 or less, 1:50 or less, 1:55 or less, 1:60 or less, 1:65 or less, 1:70 or less, 1:75 or less, 1:80 or less, 1:85 or less, 1:90 or less, 1:95 or less, 1:100 or less, 1:110 or less, 1:120 or less, 1:130 or less, 1:140 or less, 1:150 or less, 1:160 or less, 1:170 or less, 1:180 or less, 1:190 or less, or 1:200 or less. In some embodiments, the amount of a protease used in a proteolytic digestion technique is about any of 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, 1:110, 1:120, 1:130, 1:140, 1:150, 1:160, 1:170, 1:180, 1:190, or 1:200. In some embodiments, the proteolytic digestion technique comprises the use of two or more proteases, such as a combination of trypsin and LysC, and in such embodiments, the amount of each protease (such as described above) can be summed to a total amount of proteases used in a proteolytic digestion technique.

In some embodiments, the proteolytic digestion incubation time is about 20 minutes to about 36 hours, such as any of about 1 hour to about 18 hours, about 5 hours to about 24 hours, about 12 hours to about 24 hours, about 16 hours to about 20 hours, or about 12 hours to about 36 hours. In some embodiments, the proteolytic digestion incubation time is about 36 hours or less, such as about any of 32 hours or less, 30 hours or less, 28 hours or less, 26 hours or less, 24 hours or less, 22 hours or less, 20 hours or less, 19 hours or less, 18 hours or less, 17 hours or less, 16 hours or less, 15 hours or less, 14 hours or less, 13 hours or less, 12 hours or less, 11 hours or less, 10 hours or less, 9 hours or less, 8 hours or less, 7 hours or less, 6 hours or less, 5 hours or less, 4 hours or less, 3 hours or less, 2 hours or less, or 1 hours or less. In some embodiments, the proteolytic digestion incubation time is at least about 20 minutes, such as at least about any of 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 22 hours, 24 hours, 26 hours, 28 hours, 30 hours, 32 hours, 34 hours, or 36 hours. In some embodiments, the proteolytic digestion incubation time is about any of 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 22 hours, 24 hours, 26 hours, 28 hours, 30 hours, 32 hours, 34 hours, or 36 hours.

In some embodiments, the digestion incubation time is performed at a temperature of about 20° C. to about 60° C., such as any of about 20° C. to about 25° C., about 20° C. to about 30° C., about 25° C. to about 40° C., about 35° C. to about 40° C., or about 35° C. to about 50° C. In some embodiments, the digestion incubation time is performed at a temperature of at least about 20° C., such as at least about any of 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 42° C., 44° C., 46° C., 48° C., 50° C., 52° C., 54° C., 56° C., 58° C., or 60° C. In some embodiments, the digestion incubation time is performed at a temperature of about 60° C. or less, such about any of 58° C. or less, 56° C. or less, 54° C. or less, 52° C. or less, 50° C. or less, 48° C. or less, 46° C. or less, 44° C. or less, 42° C. or less, 40° C. or less, 39° C. or less, 38° C. or less, 37° C. or less, 36° C. or less, 35° C. or less, 34° C. or less, 33° C. or less, 32° C. or less, 31° C. or less, 30° C. or less, 29° C. or less, 28° C. or less, 27° C. or less, 26° C. or less, 25° C. or less, 24° C. or less, 23° C. or less, 22° C. or less, 21° C. or less, or 20° C. or less. In some embodiments, the digestion incubation time is performed at a temperature of about any of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 42° C., 44° C., 46° C., 48° C., 50° C., 52° C., 54° C., 56° C., 58° C., or 60° C. In some embodiments, the reduction incubation time is performed at a room temperature.

In some embodiments, the proteolytic digestion technique comprises a digestion incubation time of about 12 hours to about 24 hours, such as about any of 13 hours, 14 hours, 15 hours, 16 hours, 17 hours, 18 hours, 19 hours, 20 hours, 21 hours, 22 hours, 23 hours, wherein the digestion incubation time is performed at a temperature of about 20° C. to about 40° C., such about any of 22° C., 24° C., 26° C., 28° C., 30° C., 31° C., 32° C., 33° C., 34° C., 35° C., 36° C., 37° C., 38° C., 39° C. In some embodiments, the proteolytic digestion technique comprises use of an amount a protease for each of one or more proteases, e.g., trypsin and/or LysC, of about 1:15 to about 1:45, such as about any of 1:20, 1:25, 1:30, 1:35, or 1:40 (as measured based on the amount of the protease to the amount of polypeptide in a sample or a derivative thereof), and a digestion incubation time of about 12 hours to about 24 hours, such as about any of 13 hours, 14 hours, 15 hours, 16 hours, 17 hours, 18 hours, 19 hours, 20 hours, 21 hours, 22 hours, 23 hours, wherein the digestion incubation time is performed at a temperature of about 20° C. to about 40° C., such about any of 22° C., 24° C., 26° C., 28° C., 30° C., 31° C., 32° C., 33° C., 34° C., 35° C., 36° C., 37° C., 38° C., 39° C. In some embodiments, the proteolytic digestion technique is performed on a plasma sample or a derivate thereof, wherein the proteolytic digestion technique comprises a 1:40 ratio of trypsin to polypeptide in the sample of the derivative thereof and a 1:40 ratio of LysC to polypeptide in the sample or the derivative thereof. In some embodiments, the proteolytic digestion technique is performed on a serum sample or a derivate thereof, wherein the proteolytic digestion technique comprises a 1:20 ratio of trypsin.

The digestion incubation temperature can be controlled by numerous techniques and combinations thereof. In some embodiments, the digestion incubation temperature is controlled by an ambient temperature, such as room temperature. In some embodiments, the digestion incubation temperature is controlled by a water bath. In some embodiments, the digestion incubation temperature is controlled by a heat block. In some embodiments, the digestion incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the digestion incubation time temperature are controlled by a technique described herein, such as cooling at room temperature or a ramp rate.

In some embodiments, the proteolytic digestion technique comprises subjecting a sample, or a derivative thereof, e.g., an alkylated sample (including an alkylated sample quenched with a neutralizing agent), to a thermal cycle. In some embodiments, the thermal cycle comprises subjecting the sample, or a derivative thereof, to one or more of: (a) a block starting temperature (b) block set temperature (the temperature for the digestion incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 20° C. to about 50° C., including about any of 22° C., 24° C., 26° C., 28° C., 30° C., 31° C., 32° C., 33° C., 34° C., 35° C., 36° C., 37° C., 38° C., 39° C., 40° C., 42° C., 44° C., 46° C., 48° C.

In some embodiments, the thermal cycle comprises: (a) a set block temperature of about 20° C. to about 50° C., including about any of 22° C., 24° C., 26° C., 28° C., 30° C., 31° C., 32° C., 33° C., 34° C., 35° C., 36° C., 37° C., 38° C., 39° C., 40° C., 42° C., 44° C., 46° C., 48° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 35° C., such as any of about 20° C. to about 35° C., or about 20° C. to about 25° C., (b) a set block temperature of about 20° C. to about 50° C., including about any of 22° C., 24° C., 26° C., 28° C., 30° C., 31° C., 32° C., 33° C., 34° C., 35° C., 36° C., 37° C., 38° C., 39° C., 40° C., 42° C., 44° C., 46° C., 48° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

In some embodiments, the proteolytic digestion technique further comprises quenching the one or more proteolytic enzymes. In some embodiments, quenching the one or more proteolytic enzymes comprises denaturing the one or more proteolytic enzymes. In some embodiments, quenching the one or more proteolytic enzymes comprise adding an amount of an acid. In some embodiments, the acid is formic acid (FA) or trifluoroacetic acid (TFA), or a mixture thereof. In some embodiments, the amount (as assessed based on the final concentration in the sample containing solution) of the acid added is about any of 0.1% v/v, 0.2% v/v, 0.3% v/v, 0.4% v/v, 0.5% v/v, 0.6% v/v, 0.7% v/v, 0.8% v/v, 0.9% v/v, 1% v/v, 1.1% v/v, 1.2% v/v, 1.3% v/v, 1.4% v/v, 1.5% v/v, 1.6% v/v, 1.7% v/v, 1.8% v/v, 1.9% v/v, or 2% v/v.

### V. Additional Techniques

In certain aspects, the method provided herein comprise subjecting the proteolytically digested sample comprising a proteolytic glycopeptide to one or more additional steps prior to subjecting the proteolytically digested sample, or a derivative thereof, to a liquid chromatography-mass spectrometry (LC-MS) technique using a liquid chromatography system and a mass spectrometer. In some embodiments, the LC system is online with the MS (i.e., eluate from the LC system is directly introduced to the MS). In some embodiments, the one or more additional steps do not include a desalting step performed outside of the LC system (such as an offline desalting technique).

In some embodiments, the method further comprises adding a standard to the proteolytically digested sample prior to the LC-MS technique. In some embodiments, the standard is a stable isotope-internal standard (SI-IS) peptide mixture.

In some embodiments, the biological sample is not subjected to a high-abundant protein depletion technique prior to the thermal denaturation technique. For example, in some embodiments, the high-abundant protein depletion technique removes highly abundant proteins present in a blood sample, such as serum albumin.

### D. Methods for Performing a LC-MS Analysis of a Proteolytic Glycopeptide

In certain aspects, provided herein is a method for performing a LC-MS analysis on a sample comprising a proteolytic glycopeptide. In some embodiments, the liquid chromatography (LC) system is online with a mass spectrometer (i.e., proteolytic peptide species, including glycopeptides, are eluted from the LC system directing into the mass spectrometer via a mass spectrometer interface. In some embodiments, the LC technique comprises performing a chromatographic separation of one or more proteolytic peptides, including glycopeptides. In some embodiments, the one or more proteolytic peptides subjected to a chromatographic separation are obtained from a proteolytically digested sample, such as described herein. In some embodiments, the chromatographic separation is performed on a proteolytically digested sample, such as described herein, (e.g., no additional separation technique, such as a sample clean-up step, is performed to remove one or more components from proteolytically digested sample). In some embodiments, the chromatographic separation is performed on a proteolytically digested sample comprising at least about 5 mM of a buffer, such as ammonium bicarbonate. In some embodiments, the chromatographic separation is performed on a proteolytically digested sample comprising an amount, such as at least about 1 mM, of a reducing agent or a byproduct thereof, such as a stable six-membered ring with an internal disulfide bond derived from DTT. In some embodiments, the chromatographic separation is performed on a proteolytically digested sample comprising an amount, such as at least about 1 mM, of an alkylating agent or a byproduct thereof, such as iodide (I-) derived from IAA. Under certain circumstances depending on the liquid pH, DTT, the six-membered ring with an internal disulfide bond, IAA, and I may be in an ionic form and can be referred to as a salt. In addition, the salt can be a non-volatile salt that is less likely to vaporize upon entering the MS increasing the likelihood of a contaminating residue in the MS causing the need for a cleaning maintenance.

In some embodiments, the method comprises introducing the proteolytically digested sample to a LC-MS system. In some embodiments, the method comprises performing a chromatographic separation of the proteolytically digested sample. In some embodiments, the chromatography separation comprises a period of diversion (i.e., diverted from the mass spectrometer interface, e.g., to a waste receptacle) of an initial eluate from the proteolytically digested sample. In some embodiments, the initial eluate (as assessed from the sample front) diverted from the mass spectrometer is about 1 column volume of the chromatographic column to about 5 column volumes of the chromatographic column, such as any of about 1 column volumes to about 4 column volumes, about 2 column volumes to about 5 column volumes, or about 3 column volumes to about 4 column volumes. In some embodiments, the initial eluate (as assessed from the sample front) diverted from the mass spectrometer is at least about 0.5 column volumes, such as at least about any of 1 column volume, 1.5 column volumes, 2 column volumes, 2.5 column volumes, 3 column volumes, 3.5 column volumes, 4 column volumes, 4.5 column volumes, or 5 column volumes. In some embodiments, the initial eluate (as assessed from the sample front) diverted from the mass spectrometer is about 5 column volumes or less, such as about any of 4.5 column volumes or less, 4 column volumes or less, 3.5 column volumes or less, 3 column volumes or less, 2.5 column volumes or less, 2 column volumes or less, 1.5 column volumes or less, 1 column volume or less, or 0.5 column volumes or less. In some embodiments, the initial eluate (as assessed from the sample front) diverted from the mass spectrometer is about any of 0.5 column volumes, 1 column volume, 1.5 column volumes, 2 column volumes, 2.5 column volumes, 3 column volumes, 3.5 column volumes, 4 column volumes, 4.5 column volumes, or 5 column volumes.

In some embodiments, the chromatographic separation comprises a gradient separation performing using mixtures of an aqueous mobile phase and an organic mobile phase. In some embodiments, the chromatographic separation comprises isocratic period, such as a period of at least about 90%, such as at least about any of 91%, 92%, 93%, 94%, 95%, 96%, 97%, 98%, 99%, or 100%, of an aqueous mobile phase to produce the initial eluate that is diverted from the mass spectrometer interface.

In some embodiments, the LC system comprises a reversed-phase chromatography column. In some embodiments, the reversed-phase column comprises an alkyl moiety, such as C18.

The present application contemplates a diverse array of additional features of LC-MS techniques for analyzing a sample comprising a glycopeptide using a mass spectrometer. In some embodiments, the liquid chromatography system comprises a high performance liquid chromatography system. In some embodiments, the liquid chromatography system comprises an ultra-high performance liquid chromatography system. In some embodiments, the liquid chromatography system comprises a high-flow liquid chromatography system. In some embodiments, the liquid chromatography system comprises a low-flow liquid chromatography system, such as a micro-flow liquid chromatography system or a nano-flow liquid chromatography system. In some embodiments, the liquid chromatography system is coupled, such as directly interfaced, with a mass spectrometer.

In some embodiment, the mass spectrometry technique comprises an ionization technique. Ionization techniques contemplated by the present application include techniques capable of charging polypeptides and peptide products, including glycopeptides. Thus, in some embodiments, the ionization technique is electrospray ionization. In some embodiments, the ionization technique is nano-electrospray ionization. In some embodiments, the ionization technique is atmospheric pressure chemical ionization. In some embodiments, the ionization technique is atmospheric pressure photoionization.

A diverse array of mass spectrometers are contemplated as compatible with the description, include high-resolution mass spectrometers and low-resolution mass spectrometers. In some embodiments, the mass spectrometer is a time-of-flight (TOF) mass spectrometer. In some embodiments, the mass spectrometer is a quadrupole time-of-flight (Q-TOF) mass spectrometer. In some embodiments, the mass spectrometer is a quadrupole ion trap time-of-flight (QIT-TOF) mass spectrometer. In some embodiments, the mass spectrometer is an ion trap. In some embodiments, the mass spectrometer is a single quadrupole. In some embodiments, the mass spectrometer is a triple quadrupole (QQQ). In some embodiments, the mass spectrometer is an orbitrap. In some embodiments, the mass spectrometer is a quadrupole orbitrap. In some embodiments, the mass spectrometer is a fourier transform ion cyclotron resonance (FT) mass spectrometer. In some embodiments, the mass spectrometer is a quadrupole fourier transform ion cyclotron resonance (Q-FT) mass spectrometer. In some embodiments, the mass spectrometry technique comprises positive ion mode. In some embodiments, the mass spectrometry technique comprises negative ion mode. In some embodiments, the mass spectrometry technique comprises an ion mobility mass spectrometry technique.

In some embodiments, the LC-MS technique comprises processing obtained signals MS from the mass spectrometer. In some embodiments, the LC-MS technique comprises peak detection. In some embodiments, the LC-MS technique comprises determining ionization intensity of an ionized peptide product. In some embodiments, the LC-MS technique comprises determining peak height of an ionized peptide product. In some embodiments, the LC-MS technique comprises determining peak area of an ionized peptide product. In some embodiments, the LC-MS technique comprises determining peak volume of an ionized peptide product. In some embodiments, the LC-MS technique comprises identifying an ionized peptide product by amino acid sequence. In some embodiments, the LC-MS technique comprises determining the site of a post-translational modification of an ionized peptide, such as the site of a glycosylation. In some embodiments, the LC-MS technique comprises determining the glycan structure, or a characteristic thereof, of an ionized peptide product. In some embodiments, the LC-MS technique comprises manually validating the ionized peptide product acid sequence assignments. In some embodiments, the LC-MS technique comprises a quantification technique.

### E. Exemplary Methods

In some aspects, provided herein is a method for proteolytically digesting a biological sample comprising a glycoprotein to produce a proteolytic glycopeptide, the method comprising: subjecting the biological sample to a thermal denaturation technique to produce a denatured sample, wherein the thermal denaturation technique comprises subjecting the biological sample to a first thermal cycle comprising a thermal treatment of about 60° C. to about 100° C. with a hold time of at least about 1 minute, wherein the lid temperature during the first thermal cycle is the same or greater than (e.g., 0° C. to up to 20° C. greater than) the temperature of the block temperature during the first thermal cycle, such as at least about 2° C. higher than the temperature of the block temperature during the first thermal cycle, including about 5° C. to about 20° C. higher than the temperature of the block temperature during the first thermal cycle; subjecting the denatured sample to a reduction technique to produce a reduced sample, wherein the reduction technique comprises adding an amount of a reducing agent to the denatured sample and incubating for a reducing incubation time; subjecting the reduced sample to an alkylation technique to produce an alkylated sample, wherein the alkylation technique comprises adding an amount of an alkylating agent to the reduced sample and incubating substantially in the dark or in a low light condition for an alkylation incubation time, and wherein the alkylated technique comprises quenching the alkylating agent following the alkylation incubation time; and subjecting the alkylated sample to a proteolytic digestion technique to produce a proteolytically digested sample comprising the proteolytic glycopeptide, wherein the proteolytic digestion technique comprises adding an amount of one or more proteolytic enzymes and incubating for a digestion incubation time, and wherein the proteolytic digestion technique comprises quenching the one or more proteolytic enzymes following the digestion incubation time. In some embodiments, the first thermal cycle of the thermal denaturation technique comprises: (a) starting block temperature of about 15° C. to about 50° C., such as any of 15° C. to about 25° C., about 20° C. to about 30° C., or about 20° C. to about 25° C., including about any of 20° C., 21° C., 22° C., 23° C., 24° C., 25° C., (b) a set block temperature of about 60° C. to about 100° C., such about any of 60° C., 65° C., 70° C., 75° C., 80° C., 85° C., 90° C., 91° C., 92° C., 93° C., 94° C., 95° C., 96° C., 97° C., 98° C., 99° C., or 100° C., and (c) a block ending temperature of about 15° C. to about 35° C., such as any of about 20° C. to about 35° C., or about 20° C. to about 25° C. In some embodiments, the reduction technique comprises use of an amount (as assessed based on the final concentration in the sample containing solution) of a reducing agent, e.g., DTT, of about 5 mM to about 25 mM, such as any of about 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, or 24 mM, and a reduction incubation time of about 30 minutes to about 70 minutes, such as about any of 35 minutes, 40 minutes, 45 minutes, 50 minutes, 55 minutes, 60 minutes, or 65 minutes, wherein the reduction incubation time is performed at a temperature of about 50° C. to about 70° C., such about any of 55° C., 60° C., or 65° C. In some embodiments, the reduction technique comprises subjecting the denatured sample to a second thermal cycle to control temperature. In some embodiments, the second thermal cycle of the reduction technique comprises: (a) starting block temperature of about 15° C. to about 60° C., such as any of about 15° C. to about 50° C., about 20° C. to about 40° C., about 20° C. to about 30° C., or about 20° C. to about 25° C., (b) a set block temperature of about 20° C. to about 100° C., such as any of about 40° C. to about 80° C., about 50° C. to about 70° C., about 50° C. to about 60° C., about 55° C. to about 65° C., or about 60° C. to about 70° C., including about any of 50° C., 55° C., 60° C., 65° C., or 70° C., and (c) a block ending temperature of about 15° C. to about 35° C., such as any of about 20° C. to about 35° C., or about 20° C. to about 25° C. In some embodiments, the alkylation technique comprises use of an amount (containing solution based on the final concentration in the sample) of an alkylating agent, e.g., IAA, of about 15 mM to about 40 mM, such as any of about 20 mM, 20.5 mM, 21 mM, 21.5 mM, 22 mM, 22.5 mM, 23 mM, 23.5 mM, 24 mM, 24.5 mM, or 25 mM, 26 mM, 27 mM, 28 mM, 29 mM, 30 mM, 31 mM, 32 mM, 33 mM, 34 mM, or 35 mM, and an alkylation incubation time of about 5 minutes to about 60 minutes, such as about any of 15 minutes, 20 minutes, 25 minutes, 30 minutes, 35 minutes, or 40 minutes, wherein the alkylation incubation time is performed at a temperature of about 15° C. to about 30° C., such about any of 20° C., 21° C., 22° C., 23° C., 24° C., or 25° C. In some embodiments, the alkylation technique comprises subjecting the reduced sample to a third thermal cycle to control temperature. In some embodiments, the third thermal cycle of the alkylation technique comprises: (a) starting block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., 25° C., (b) a set block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., 25° C., and (c) a block ending temperature of about 15° C. to about 35° C., such as any of about 20° C. to about 35° C., or about 20° C. to about 25° C. In some embodiments, the proteolytic digestion technique comprises use of an amount a protease for each of one or more proteases, e.g., trypsin and/or LysC, of about 1:15 to about 1:45, such as about any of 1:20, 1:25, 1:30, 1:35, or 1:40, and a digestion incubation time of about 12 hours to about 24 hours, such as about any of 13 hours, 14 hours, 15 hours, 16 hours, 17 hours, 18 hours, 19 hours, 20 hours, 21 hours, 22 hours, 23 hours, wherein the digestion incubation time is performed at a temperature of about 20° C. to about 40° C., such about any of 22° C., 24° C., 26° C., 28° C., 30° C., 31° C., 32° C., 33° C., 34° C., 35° C., 36° C., 37° C., 38° C., 39° C. In some embodiments, the proteolytic digestion technique comprises subjecting the alkylated sample to a fourth thermal cycle to control temperature. In some embodiments, the fourth thermal cycle of the proteolytic digestion comprises: (a) starting block temperature of about 15° C. to about 35° C., such as any of about 20° C. to about 35° C., or about 20° C. to about 25° C., (b) a set block temperature of about 20° C. to about 50° C., including about any of 22° C., 24° C., 26° C., 28° C., 30° C., 31° C., 32° C., 33° C., 34° C., 35° C., 36° C., 37° C., 38° C., 39° C., 40° C., 42° C., 44° C., 46° C., 48° C., and (c) a block ending temperature of about 15° C. to about 35° C., such as any of about 20° C. to about 35° C., or about 20° C. to about 25° C. It is to be noted that step numbering, such as first, second, third, and fourth thermal cycle, is not intended to suggest an order of performing the steps described herein.

In other aspects, provided herein is a method for performing a liquid chromatography-mass spectrometry analysis of a proteolytic glycopeptide derived from a biological sample comprising a glycoprotein, the method comprising: subjecting the biological sample to a thermal denaturation technique to produce a denatured sample followed by a proteolytic digestion technique to produce a proteolytically digested sample comprising the glycopeptide, wherein the thermal denaturation technique subjects the biological sample to a thermal cycle comprising a thermal treatment of about 60° C. to about 100° C. with a hold time of at least about 1 minute, wherein the lid temperature during the thermal cycle is the same or greater than (e.g., 0° C. to up to 20° C. greater than) the temperature of the block temperature during the thermal cycle, such as at least about 2° C. higher than the temperature of the block temperature during the thermal cycle, including about 5° C. to about 20° C. higher than the temperature of the block temperature during the first thermal cycle, wherein the proteolytic digestion technique comprises adding an amount of one or more proteolytic enzymes and incubating for a digestion incubation time, and wherein the digestion technique comprises quenching the one or more proteolytic enzymes following the digestion incubation time; introducing the proteolytically digested sample to a liquid chromatography (LC) system of a LC-MS system; and performing a LC separation to introduce the proteolytic glycopeptide to a mass spectrometer (MS) system, wherein the LC separation comprises a period of diversion of an initial eluate comprising a buffer salt, and wherein the LC system comprises a reversed-phase chromatography column. In some embodiments, the thermal denaturation technique comprises subjecting the biological sample, or a derivative thereof, to a first thermal cycle to control temperature. In some embodiments, the first thermal cycle of the thermal denaturation technique comprises: (a) starting block temperature of about 15° C. to about 50° C., such as any of 15° C. to about 25° C., about 20° C. to about 30° C., or about 20° C. to about 25° C., including about any of 20° C., 21° C., 22° C., 23° C., 24° C., 25° C., (b) a set block temperature of about 60° C. to about 100° C., such about any of 60° C., 65° C., 70° C., 75° C., 80° C., 85° C., 90° C., 91° C., 92° C., 93° C., 94° C., 95° C., 96° C., 97° C., 98° C., 99° C., or 100° C., and (c) a block ending temperature of about 15° C. to about 35° C., such as any of about 20° C. to about 35° C., or about 20° C. to about 25° C. In some embodiments, the proteolytic digestion technique comprises use of an amount a protease for each of one or more proteases, e.g., trypsin and/or LysC, of about 1:15 to about 1:45, such as about any of 1:20, 1:25, 1:30, 1:35, or 1:40, and a digestion incubation time of about 12 hours to about 24 hours, such as about any of 13 hours, 14 hours, 15 hours, 16 hours, 17 hours, 18 hours, 19 hours, 20 hours, 21 hours, 22 hours, 23 hours, wherein the digestion incubation time is performed at a temperature of about 20° C. to about 40° C., such about any of 22° C., 24° C., 26° C., 28° C., 30° C., 31° C., 32° C., 33° C., 34° C., 35° C., 36° C., 37° C., 38° C., 39° C. In some embodiments, the proteolytic digestion technique comprises subjecting the alkylated sample to a second thermal cycle to control temperature. In some embodiments, the second thermal cycle of the proteolytic digestion comprises: (a) starting block temperature of about 15° C. to about 35° C., such as any of about 20° C. to about 35° C., or about 20° C. to about 25° C., (b) a set block temperature of about 20° C. to about 50° C., including about any of 22° C., 24° C., 26° C., 28° C., 30° C., 31° C., 32° C., 33° C., 34° C., 35° C., 36° C., 37° C., 38° C., 39° C., 40° C., 42° C., 44° C., 46° C., 48° C., and (c) a block ending temperature of about 15° C. to about 35° C., such as any of about 20° C. to about 35° C., or about 20° C. to about 25° C. In some embodiments, the chromatography separation comprises a period of diversion (i.e., diverted from the mass spectrometer interface, e.g., to a waste receptacle) of an initial eluate from the proteolytically digested sample. In some embodiments, the initial eluate (as assessed from the sample front) diverted from the mass spectrometer is about 1 column volume to about 5 column volumes, including about any of 0.5 column volumes, 1 column volume, 1.5 column volumes, 2 column volumes, 2.5 column volumes, 3 column volumes, 3.5 column volumes, 4 column volumes, 4.5 column volumes, or 5 column volumes. It is to be noted that step numbering, such as first and second thermal cycle, is not intended to suggest an order of performing the steps described herein.

In some aspects, provided herein is a method for performing a LC-MS analysis of a proteolytic glycopeptide derived from a biological sample comprising a glycoprotein, the method comprising: subjecting the biological sample to a thermal denaturation technique to produce a denatured sample, wherein the thermal denaturation technique comprises subjecting the biological sample to a first thermal cycle comprising a thermal treatment of about 60° C. to about 100° C. with a hold time of at least about 1 minute, wherein the lid temperature during the first thermal cycle is the same or greater than (e.g., 0° C. to up to 20° C. greater than) the temperature of the block temperature during the first thermal cycle, such as at least about 2° C. higher than the temperature of the block temperature during the first thermal cycle, including about 5° C. to about 20° C. higher than the temperature of the block temperature during the first thermal cycle; subjecting the denatured sample to a reduction technique to produce a reduced sample, wherein the reduction technique comprises adding an amount of a reducing agent to the denatured sample and incubating for a reducing incubation time; subjecting the reduced sample to an alkylation technique to produce an alkylated sample, wherein the alkylation technique comprises adding an amount of an alkylating agent to the reduced sample and incubating substantially in the dark or in a low light condition for an alkylation incubation time, and wherein the alkylated technique comprises quenching the alkylating agent following the alkylation incubation time; subjecting the alkylated sample to a proteolytic digestion technique to produce a proteolytically digested sample comprising the proteolytic glycopeptide, wherein the proteolytic digestion technique comprises adding an amount of one or more proteolytic enzymes and incubating for a digestion incubation time, and wherein the proteolytic digestion technique comprises quenching the one or more proteolytic enzymes following the digestion incubation time; and introducing the proteolytically digested sample to a liquid chromatography (LC) system of a LC-MS system; and performing a LC separation to introduce the proteolytic glycopeptide to a mass spectrometer (MS) system, wherein the LC separation comprises a period of diversion of an initial eluate comprising a buffer salt, and wherein the LC system comprises a reversed-phase chromatography column. In some embodiments, the first thermal cycle of the thermal denaturation technique comprises: (a) starting block temperature of about 15° C. to about 50° C., such as any of 15° C. to about 25° C., about 20° C. to about 30° C., or about 20° C. to about 25° C., including about any of 20° C., 21° C., 22° C., 23° C., 24° C., 25° C., (b) a set block temperature of about 60° C. to about 100° C., such about any of 60° C., 65° C., 70° C., 75° C., 80° C., 85° C., 90° C., 91° C., 92° C., 93° C., 94° C., 95° C., 96° C., 97° C., 98° C., 99° C., or 100° C., and (c) a block ending temperature of about 15° C. to about 35° C., such as any of about 20° C. to about 35° C., or about 20° C. to about 25° C. In some embodiments, the reduction technique comprises use of an amount (as assessed based on the final concentration in the sample containing solution) of a reducing agent, e.g., DTT, of about 5 mM to about 25 mM, such as any of about 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, or 24 mM, and a reduction incubation time of about 30 minutes to about 70 minutes, such as about any of 35 minutes, 40 minutes, 45 minutes, 50 minutes, 55 minutes, 60 minutes, or 65 minutes, wherein the reduction incubation time is performed at a temperature of about 50° C. to about 70° C., such about any of 55° C., 60° C., or 65° C. In some embodiments, the reduction technique comprises subjecting the denatured sample to a second thermal cycle to control temperature. In some embodiments, the second thermal cycle of the reduction technique comprises: (a) starting block temperature of about 15° C. to about 60° C., such as any of about 15° C. to about 50° C., about 20° C. to about 40° C., about 20° C. to about 30° C., or about 20° C. to about 25° C., (b) a set block temperature of about 20° C. to about 100° C., such as any of about 40° C. to about 80° C., about 50° C. to about 70° C., about 50° C. to about 60° C., about 55° C. to about 65° C., or about 60° C. to about 70° C., including about any of 50° C., 55° C., 60° C., 65° C., or 70° C., and (c) a block ending temperature of about 15° C. to about 35° C., such as any of about 20° C. to about 35° C., or about 20° C. to about 25° C. In some embodiments, the alkylation technique comprises use of an amount (containing solution based on the final concentration in the sample) of an alkylating agent, e.g., IAA, of about 15 mM to about 40 mM, such as any of about 20 mM, 20.5 mM, 21 mM, 21.5 mM, 22 mM, 22.5 mM, 23 mM, 23.5 mM, 24 mM, 24.5 mM, or 25 mM, 26 mM, 27 mM, 28 mM, 29 mM, 30 mM, 31 mM, 32 mM, 33 mM, 34 mM, or 35 mM, and an alkylation incubation time of about 5 minutes to about 60 minutes, such as about any of 15 minutes, 20 minutes, 25 minutes, 30 minutes, 35 minutes, or 40 minutes, wherein the alkylation incubation time is performed at a temperature of about 15° C. to about 30° C., such about any of 20° C., 21° C., 22° C., 23° C., 24° C., or 25° C. In some embodiments, the alkylation technique comprises subjecting the reduced sample to a third thermal cycle to control temperature. In some embodiments, the third thermal cycle of the alkylation technique comprises: (a) starting block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., 25° C., (b) a set block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., 25° C., and (c) a block ending temperature of about 15° C. to about 35° C., such as any of about 20° C. to about 35° C., or about 20° C. to about 25° C. In some embodiments, the proteolytic digestion technique comprises use of an amount a protease for each of one or more proteases, e.g., trypsin and/or LysC, of about 1:15 to about 1:45, such as about any of 1:20, 1:25, 1:30, 1:35, or 1:40, and a digestion incubation time of about 12 hours to about 24 hours, such as about any of 13 hours, 14 hours, 15 hours, 16 hours, 17 hours, 18 hours, 19 hours, 20 hours, 21 hours, 22 hours, 23 hours, wherein the digestion incubation time is performed at a temperature of about 20° C. to about 40° C., such about any of 22° C., 24° C., 26° C., 28° C., 30° C., 31° C., 32° C., 33° C., 34° C., 35° C., 36° C., 37° C., 38° C., 39° C. In some embodiments, the proteolytic digestion technique comprises subjecting the alkylated sample to a fourth thermal cycle to control temperature. In some embodiments, the fourth thermal cycle of the proteolytic digestion comprises: (a) starting block temperature of about 15° C. to about 35° C., such as any of about 20° C. to about 35° C., or about 20° C. to about 25° C., (b) a set block temperature of about 20° C. to about 50° C., including about any of 22° C., 24° C., 26° C., 28° C., 30° C., 31° C., 32° C., 33° C., 34° C., 35° C., 36° C., 37° C., 38° C., 39° C., 40° C., 42° C., 44° C., 46° C., 48° C., and (c) a block ending temperature of about 15° C. to about 35° C., such as any of about 20° C. to about 35° C., or about 20° C. to about 25° C. In some embodiments, the chromatography separation comprises a period of diversion (i.e., diverted from the mass spectrometer interface, e.g., to a waste receptacle) of an initial eluate from the proteolytically digested sample. In some embodiments, the initial eluate (as assessed from the sample front) diverted from the mass spectrometer is about 1 column volume to about 5 column volumes, including about any of 0.5 column volumes, 1 column volume, 1.5 column volumes, 2 column volumes, 2.5 column volumes, 3 column volumes, 3.5 column volumes, 4 column volumes, 4.5 column volumes, or 5 column volumes. It is to be noted that step numbering, such as first and second thermal cycle, is not intended to suggest an order of performing the steps described herein. In some embodiments, the method does not include use of a separate clean-up step performed prior to the LC-MS technique, such as a desalting step. It is to be noted that step numbering, such as first, second, third, and fourth thermal cycle, is not intended to suggest an order of performing the steps described herein.

### F. Samples and Components Thereof

The methods provided herein are contemplated to be suitable for analyzing a diverse array of samples, such as biological samples. In some embodiments, the sample is a blood sample, such as a whole blood sample. In some embodiments, the sample is a plasma sample. In some embodiments, the sample is a serum sample. In some embodiments, the sample is a tissue sample. Plasma is a fluid component of blood that is obtained when a clotting-prevention agent is added to whole blood and then the tube is centrifuged to separate the cellular material. The upper lighter colored liquid layer in the tube is removed as plasma. Common anti-coagulant agents are EDTA (ethylenediaminetetraacetic acid), heparin, and citrate. Serum is a fluid obtained when whole blood is allowed to clot in a tube and then centrifuged so that the clotted blood, including red cells, are at the bottom of the collection tube, leaving a straw-colored liquid above the clot. The straw-colored liquid in the tube is removed as serum.

The methods provided herein are particularly useful for the analysis of biological samples comprising a glycoprotein, such as to generate glycopeptide containing specimens for analysis with a mass spectrometer. The methods provided herein, in some embodiments, enable the analysis of glycopeptides that elute during early or late phases of a reversed-phase chromatographic separation and are typically missed during conventional mass spectrometry approaches. For the situation where a sample contains hydrophilic salts and hydrophilic glycopeptides, it can be challenging to desalt the sample with a C18 sample phase extraction material without removing a significant portion of the hydrophilic glycopeptides that are needed for an analysis of the sample. For example, glycopeptides with an overall hydrophilic character may elute from a reversed-phase material, such as in a desalting column, and are washed away or not introduced to the mass spectrometer during a data acquisition phase of a mass spectrometry technique. In some embodiments, glycopeptides with an overall hydrophobic character may have a high affinity for a reversed-phase material, such as in a desalting column or a chromatography column, and are not properly eluted from a desalting column or during a data acquisition portion of a mass spectrometry technique.

In some embodiments, the method comprises an upstream sample preparation technique, such as for obtaining plasma or serum from a blood sample, performed prior to methods for proteolytically digesting a sample. In some embodiments, the upstream sample preparation technique comprises a cell lysis step. In some embodiments, the upstream sample preparation technique comprises a filtration step. In some embodiments, the upstream sample preparation technique comprises a dilution step. In some embodiments, the upstream sample preparation technique comprises a protein concentration determination step.

In some embodiments, the sample is obtained from an individual. In some embodiments, the sample is obtained from a human individual.

### G. Systems, Kits, and Compositions

In certain aspects, contemplated herein are systems, kits, and compositions useful for performing the methods described herein. In some embodiments, provided herein is a system, kit, and/or composition useful for performing a proteolytic digestion of a biological sample comprising a glycoprotein as described herein. In some embodiments, provided herein is a system, kit, and/or composition useful for performing a LC-MS analysis of a proteolytic glycopeptide as described herein. In some embodiments, provided herein is a system, kit, and/or composition useful for performing a proteolytic digestion of a biological sample comprising a glycoprotein followed by LC-MS analysis of the proteolytic glycopeptide produced therefrom.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### Section 2—Reversed-Phase Proteolytic Digestion Clean-Up Techniques for Samples Containing a Glycosylated Polypeptide

Provided herein, in certain aspects, are methods for processing a proteolytically digested sample to produce a processed sample suitable for use in a liquid chromatography-mass spectrometry (LC-MS) analysis, wherein the methods comprise one or more, including any combinations thereof, techniques taught herein for subjecting the proteolytically digested sample to a solid phase extraction column comprising a reversed-phase medium or subjecting the reversed-phase medium to a wash buffer. The disclosure of the present application is based on the inventors' unique perspective and unexpected findings regarding methods for processing a proteolytically digested sample that provide an improved LC-MS analysis of glycoproteins and glycopeptides. The methods taught herein were demonstrated to significantly reduce the loss of proteolytic peptides (including proteolytic glycopeptides) during the sample clean-up processing steps, and lead to improved reproducibility, accuracy, and quantification. The methods taught herein are also amenable to automation, thus providing robust and high-throughput methods for improving the LC-MS analysis of glycoproteins and glycopeptides. Such results represent a significant advancement in the ability to use glycoproteins in the study of human physiology, such as for disease diagnosis and treatment monitoring.

Thus, in some aspects, provided herein is a method for processing a proteolytically digested sample to produce a processed sample suitable for use in a liquid chromatography-mass spectrometry (LC-MS) analysis, wherein the proteolytically digested sample comprises a plurality of proteolytic polypeptides comprising at least one proteolytic glycopeptide, the method comprising: performing one or more of the following: (a) subjecting the proteolytically digested sample to a solid phase extraction column comprising a reversed-phase medium according to one or more conditions to associate at least a portion of the plurality of proteolytic polypeptides with the reversed-phase medium, the one or more conditions comprising: (i) a polypeptide loading amount of about 50% or less of a binding capacity of the reversed-phase medium, wherein the binding capacity of the reversed-phase medium is based on an insulin load having 10% or less breakthrough; or (ii) a polypeptide loading concentration of about 0.6 μg/μL or less; or (b) subjecting the reversed-phase medium comprising the associated proteolytic polypeptides to a wash buffer at a wash flow rate of about 0.1 column volumes/minute to about 2 column volumes/minute; and subjecting the reversed-phase medium comprising the associated proteolytic polypeptides to an elution buffer to produce the processed sample.

### C2. Methods for Processing a Proteolytically Digested Sample

Provided herein, in certain aspects, are methods of processing a proteolytically digested sample using a solid phase extraction column comprising a reversed-phase material. As described in the instant application, in some embodiments, the techniques taught herein for subjecting a proteolytically digested sample to the solid phase extraction column and/or subjecting the reversed-phase medium comprising associated proteolytic polypeptides to a wash buffer provide improved LC-MS analyses of proteolytic glycopeptides. In some embodiments, the method comprises subjecting a proteolytically digested sample to a solid phase extraction column comprising a reversed-phase medium to associate at least a portion of a plurality of proteolytic polypeptides with the reversed-phase medium, wherein the polypeptide loading amount used for subjecting the reversed-phase medium to the portion of the plurality of proteolytic polypeptides is about 50% or less of a binding capacity of the reversed-phase medium, and wherein the binding capacity of the reversed-phase medium is based on an insulin load having 10% or less breakthrough. In some embodiments, the method comprises subjecting a proteolytically digested sample to a solid phase extraction column comprising a reversed-phase medium to associate at least a portion of a plurality of proteolytic polypeptides with the reversed-phase medium, wherein the polypeptide loading concentration used for subjecting the proteolytic polypeptides to the reversed-phase medium is about 0.6 μg/μL or less. In some embodiments, the method comprises subjecting a reversed-phase medium comprising the associated proteolytic polypeptides to a wash buffer at a wash flow rate of about 0.1 column volumes/minute to about 2 column volumes/minute.

In the following sections, additional description of the various aspects of the methods for processing a proteolytically digested sample is provided. Such description in a modular fashion is not intended to limit the scope of the disclosure, and based on the teachings provided herein one of ordinary skill in the art will readily appreciate that certain modules can be integrated, at least in part. The section heading used herein are for organizational purposes only and are not to be construed as limiting the subject matter described.

### I. Polypeptide Loading Amounts

In certain aspects, provided herein is a method for processing a proteolytically digested sample to produce a processed sample suitable for use in a liquid chromatography-mass spectrometry (LC-MS) analysis, wherein the method comprises subjecting the proteolytically digested sample to a solid phase extraction column comprising a reversed-phase medium according to a desired polypeptide loading amount based on the binding capacity of the reversed-phase medium. In some embodiments, the method comprises subjecting a proteolytically digested sample to a solid phase extraction column comprising a reversed-phase medium to associate at least a portion of a plurality of proteolytic polypeptides with the reversed-phase medium, wherein the polypeptide loading amount used for subjecting the reversed-phase medium to the portion of the plurality of proteolytic polypeptides is 50% or less of a binding capacity of the reversed-phase medium, and wherein the binding capacity of the reversed-phase medium is based on an insulin load having 10% or less breakthrough.

The polypeptide loading amounts encompassed herein may be described using a number of approaches, e.g., a percentage of the total binding capacity as defined by a known relevant binding capacity of a solid phase extraction column, or an absolute amount of polypeptide loaded onto a solid phase extraction column. One of ordinary skill in the art will readily understand converting between different forms of the description provided herein, and determining a binding capacity of a solid phase extraction column if not already known.

In some embodiments, the polypeptide loading amount is about 1% to about 50%, such as any of about 5% to about 50%, about 7.5% to about 50%, about 7.5% to about 25%, about 15% to about 50%, about 15% to about 25%, of a binding capacity of a reversed-phase medium of a solid phase extraction column. In some embodiments, the polypeptide loading amount is about 50% or less, such as any of 45% or less, 40% or less, 35% or less, 30% or less, 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, 10% or less, 9.5% or less, 9% or less, 8.5% or less, 8% or less, or 7.5% or less, of a binding capacity of a reversed-phase medium of a solid phase extraction column. In some embodiments, the polypeptide loading amount is about 7.5%, 8%, 8.5%, 9%, 9.5%, 10%, 11%, 12%, 13%, 14%, 15%, 16%, 17%, 18%, 19%, 20%, 21%, 22%, 23%, 24%, 25%, 30%, 35%, 40%, 45%, or 50%, of a binding capacity of a reversed-phase medium of a solid phase extraction column. In any of the embodiments above, the binding capacity of the reversed-phase medium is based on an insulin load having 10% or less breakthrough. In any of the embodiments above, the binding capacity of the reversed-phase medium of the solid phase extraction column is about 400 μg.

In some embodiments, the polypeptide loading amount is about 1% to about 50%, such as any of about 5% to about 50%, about 7.5% to about 50%, about 7.5% to about 25%, about 15% to about 50%, about 15% to about 25%, of a binding capacity of a reversed-phase medium of a solid phase extraction column, wherein the binding capacity of the reversed-phase medium of the solid phase extraction column is about 400 μg. In some embodiments, the polypeptide loading amount is about 50% or less, such as any of 45% or less, 40% or less, 35% or less, 30% or less, 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, 10% or less, 9.5% or less, 9% or less, 8.5% or less, 8% or less, or 7.5% or less, of a binding capacity of a reversed-phase medium of a solid phase extraction column, wherein the binding capacity of the reversed-phase medium of the solid phase extraction column is about 400 μg. In some embodiments, the polypeptide loading amount is about 7.5%, 8%, 8.5%, 9%, 9.5%, 10%, 11%, 12%, 13%, 14%, 15%, 16%, 17%, 18%, 19%, 20%, 21%, 22%, 23%, 24%, 25%, 30%, 35%, 40%, 45%, or 50%, of a binding capacity of a reversed-phase medium of a solid phase extraction column, wherein the binding capacity of the reversed-phase medium of the solid phase extraction column is about 400 μg. In any of the embodiments above, the binding capacity of the reversed-phase medium is based on an insulin load having 10% or less breakthrough.

In some embodiments, the polypeptide loading amount is about 30 μg to about 200 μg, such as any of about 30 μg to about 100 μg, about 30 μg to about 60 μg to about 200 μg, or about 60 μg to about 100 μg, wherein the binding capacity of the reversed-phase medium of the solid phase extraction column is about 400 μg. In some embodiments, the polypeptide loading amount is about 200 μg or less, such as any of 175 μg or less, 150 μg or less, 125 μg or less, 100 μg or less, 95 μg or less, 90 μg or less, 85 μg or less, 80 μg or less, 75 μg or less, 70 μg or less, 65 μg or less, 60 μg or less, 55 μg or less, 50 μg or less, 45 μg or less, 40 μg or less, 35 μg or less, or 30 μg or less, wherein the binding capacity of the reversed-phase medium of the solid phase extraction column is about 400 μg. In some embodiments, the polypeptide loading amount about any of 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, or 200 μg, wherein the binding capacity of the reversed-phase medium of the solid phase extraction column is about 400 μg. In any of the embodiments above, the binding capacity of the reversed-phase medium is based on an insulin load having 10% or less breakthrough.

In some embodiments, the polypeptide loading amount is about 30 μg to about 200 μg, such as any of about 30 μg to about 100 μg, about 30 μg to about 60 μg to about 200 μg, or about 60 μg to about 100 μg, wherein the solid phase extraction column comprises a volume of about 5 μL of a reversed-phase medium. In some embodiments, the polypeptide loading amount is about 200 μg or less, such as any of 175 μg or less, 150 μg or less, 125 μg or less, 100 μg or less, 95 μg or less, 90 μg or less, 85 μg or less, 80 μg or less, 75 μg or less, 70 μg or less, 65 μg or less, 60 μg or less, 55 μg or less, 50 μg or less, 45 μg or less, 40 μg or less, 35 μg or less, or 30 μg or less, wherein the solid phase extraction column comprises a volume of about 5 μL of a reversed-phase medium. In some embodiments, the polypeptide loading amount about any of 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, or 200 μg, wherein the solid phase extraction column comprises a volume of about 5 μL of a reversed-phase medium. In any of the embodiments above, the binding capacity of the reversed-phase medium of the solid phase extraction column is about 400 μg. In any of the embodiments above, the binding capacity of the reversed-phase medium is based on an insulin load having 10% or less breakthrough.

The solid phase extraction columns described herein may comprise a reversed-phase medium, or an amount thereof, encompassing a range of binding capacities for the solid phase extraction column. In some embodiments, the solid phase extraction column comprises a binding capacity of about 1 μg to about 1,000 μg, such as any of about 100 μg to about 500 μg, about 200 μg to about 500 μg, about 300 μg to about 500 μg, about 350 μg to about 500 μg, or about 350 μg to about 750 μg, such as assessed using a polypeptide or a mixture thereof, including insulin. In some embodiments, the binding capacity is based on the amount of a polypeptide (including mixtures of polypeptides) that can be associated with the reversed-phase medium of a solid phase extraction column prior to occurrence of breakthrough (loss of polypeptides in the load that occurs during a binding phase such that a portion of the polypeptide is not captured by the reversed-phase medium) 10% or more. In some embodiments, the binding capacity of the reversed-phase medium is based on a polypeptide load having 10% or less, such as any of 9% or less, 8% or less, 7% or less, 6% or less, 5% or less, 4% or less, 3% or less, 2% or less, or 1% or less, breakthrough.

Polypeptide amounts described herein may be absolute or estimated amounts. In some embodiments, the amount of polypeptide content in a sample, or a derivative thereof, is a measured directly from said sample, or the derivative thereof, e.g., using a BCA quantification assay or a UV-VIS measurement at 280 nm. In some embodiments, the amount of polypeptide content in a sample, or a derivative thereof, is estimated based on a known, including reference standard, value for polypeptide content in the sample based on the origin of the sample, e.g., such as based on a known standard polypeptide concentration in human plasma or serum.

### II. Polypeptide Loading Concentrations

In certain aspects, provided herein is a method for processing a proteolytically digested sample to produce a processed sample suitable for use in a liquid chromatography-mass spectrometry (LC-MS) analysis, wherein the method comprises subjecting the proteolytically digested sample to a solid phase extraction column comprising a reversed-phase medium according to a desired polypeptide loading concentration. In some embodiments, the method comprises subjecting a proteolytically digested sample to a solid phase extraction column comprising a reversed-phase medium to associate at least a portion of a plurality of proteolytic polypeptides with the reversed-phase medium, wherein the polypeptide loading concentration used for subjecting the proteolytic polypeptides to the reversed-phase medium is about 0.6 μg/μL or less.

In some embodiments, the polypeptide loading concentration used for subjecting polypeptides of the proteolytically digested sample to a reversed-phase medium of a solid phase extraction column is about 0.1 μg/μL to about 1 μg/μL, such as any of about 0.25 μg/μL to about 1 μg/μL, about 0.25 μg/μL to about 0.75 μg/μL, or about 0.3 μg/μL to about 0.6 μg/μL. In some embodiments, the polypeptide loading concentration used for subjecting polypeptides of the proteolytically digested sample to a reversed-phase medium of a solid phase extraction column is about 1 μg/μL or less, such as about any of 0.95 μg/μL or less, 0.9 μg/μL or less, 0.85 μg/μL or less, 0.8 μg/μL or less, 0.75 μg/μL or less, 0.7 μg/μL or less, 0.65 μg/μL or less, 0.6 μg/μL or less, 0.55 μg/μL or less, 0.5 μg/μL or less, 0.45 μg/μL or less, 0.4 μg/μL or less, 0.35 μg/μL or less, or 0.3 μg/μL or less. In some embodiments, the polypeptide loading concentration used for subjecting polypeptides of the proteolytically digested sample to a reversed-phase medium of a solid phase extraction column is about any of 1 μg/μL 0.95 μg/μL, 0.9 μg/μL, 0.85 μg/μL, 0.8 μg/μL, 0.75 μg/μL, 0.7 μg/μL, 0.65 μg/μL, 0.6 μg/μL, 0.55 μg/μL, 0.5 μg/μL, 0.45 μg/μL, 0.4 μg/μL, 0.35 μg/μL, or 0.3 μg/μL.

In some embodiments, the polypeptides of the proteolytically digested sample for subjecting to the solid phase extraction column are in a volume of about 50 μL to about 500 μL, such as any of about 50 μL to about 300 μL, about 50 μL to about 250 μL, or about 100 μL to about 200 μL. In some embodiments, the polypeptides of the proteolytically digested sample for subjecting to the solid phase extraction column are in a volume of at least about 50 μL, such as at least about any of 75 μL, 100 μL, 125 μL, 150 μL, 175 μL, 200 μL, 225 μL, 250 μL, 275 μL, 300 μL, 325 μL, 350 μL, 375 μL, 400 μL, 425 μL, 450 μL, 475 μL, or 500 μL. In some embodiments, the polypeptides of the proteolytically digested sample for subjecting to the solid phase extraction column are in a volume that is at least about 40%, such as at least about any of 45%, 50%, 55%, 60%, 65%, 70%, 75%, 80%, 85%, 90%, or 95%, of the upper range recommended for a solid phase extraction column, such as per manufacturer's instructions. In some embodiments, the volume loaded onto the solid phase extraction column comprises a loading buffer.

As discussed herein, polypeptide amounts described herein may be absolute or estimated amounts. In some embodiments, the amount of polypeptide content in a sample, or a derivative thereof, is a measured directly from said sample, or the derivative thereof, e.g., using a BCA quantification assay. In some embodiments, the amount of polypeptide content in a sample, or a derivative thereof, is estimated based on a known, including reference standard, value for polypeptide content in the sample based on the origin of the sample, e.g., such as based on a known standard polypeptide concentration in human plasma or serum.

### III. Wash Buffer Flow Rates

In certain aspects, provided herein is a method for processing a proteolytically digested sample to produce a processed sample suitable for use in a liquid chromatography-mass spectrometry (LC-MS) analysis, wherein the method comprises subjecting a reversed-phase medium comprising the associated proteolytic polypeptides to a wash buffer at a desired wash flow rate. In some embodiments, the method comprises subjecting a reversed-phase medium comprising the associated proteolytic polypeptides to a wash buffer at a wash flow rate of about 0.1 column volumes/minute to about 2 column volumes/minute.

In some embodiments, the wash flow rate of the wash buffer is about 0.1 column volumes/minute to about 2 column volumes/minute, such as any of about 0.1 column volumes/minute to about 0.5 column volumes/minute, about 0.25 column volumes/minute to about 2 column volumes/minute, about 0.25 column volumes/minute to about 1.5 column volumes/minute, or about 0.3 column volumes/minute to about 1 column volumes/minute. In some embodiments, the wash flow rate of the wash buffer is less than about 2, such as less than about any of 1.9 column volumes/minute, 1.8 column volumes/minute, 1.7 column volumes/minute, 1.6 column volumes/minute, 1.5 column volumes/minute, 1.4 column volumes/minute, 1.3 column volumes/minute, 1.2 column volumes/minute, 1.1 column volumes/minute, 1 column volume/minute, 0.9 column volumes/minute, 0.8 column volumes/minute, 0.7 column volumes/minute, 0.6 column volumes/minute, 0.5 column volumes/minute, 0.4 column volumes/minute, 0.3 column volumes/minute or 0.2 column volumes/minute. In some embodiments, the wash flow rate of the wash buffer is about any of 0.1 column volumes/minute, 0.2 column volumes/minute, 0.3 column volumes/minute, 0.4 column volumes/minute, 0.5 column volumes/minute, 0.6 column volumes/minute, 0.7 column volumes/minute, 0.8 column volumes/minute, 0.9 column volumes/minute, 1 column volume/minute, 1.1 column volumes/minute, 1.2 column volumes/minute, 1.3 column volumes/minute, 1.4 column volumes/minute, 1.5 column volumes/minute, 1.6 column volumes/minute, 1.7 column volumes/minute, 1.8 column volumes/minute, 1.9 column volumes/minute, or 2 column volumes/minute. In some embodiments, the column volume refers to the volume occupied by the reversed-phase media within the solid phase extraction column or cartridge.

In some embodiments, the wash flow rate of the wash buffer is about 1 μL/minute to about 10 μL/minute, such as about 2 μL/minute to about 8 μL/minute, about 2 μL/minute to about 5 L/minute, or about 2 μL/minute to about 4 μL/minute. In some embodiments, the wash flow rate of the wash buffer is about 10 μL/minute or less, such as any of about 9 μL/minute or less, 8 μL/minute or less, 7 μL/minute or less, 6 μL/minute or less, 5 μL/minute or less, 4 μL/minute or less, 3 μL/minute or less, 2 μL/minute or less, or 1 μL/minute or less. In some embodiments, the wash flow rate of the wash buffer is about any of 1 μL/minute, 2 μL/minute, 3 μL/minute, 4 μL/minute, 5 μL/minute, 6 μL/minute, 7 μL/minute, 8 L/minute, 9 μL/minute, or 10 μL/minute. In any of the embodiments above, the column volume is about 5 μL.

In some embodiments, the method comprises subjecting the reversed-phased medium comprising the associated proteolytic polypeptides to a wash buffer, wherein the total wash buffer applied to the reversed-phase medium is about 1 to about 50 column volumes. In some embodiments, the total wash buffer applied to the reversed-phase medium is about 50 or fewer, such as any of 45 or fewer, 40 or fewer, 35 or fewer, 30 or fewer, 25 or fewer, 20 or fewer, 15 or fewer, 10 or fewer, or 5 or fewer, column volumes.

### IV. Reversed-Phase Mediums of Solid Phase Extraction Columns

The methods provided herein, in certain aspects, involve solid phase extraction columns comprising a reversed-phase medium. Generally speaking, reversed-phase media comprise a relatively hydrophobic stationary phase configured to associate with proteolytic polypeptides, wherein relatively hydrophilic compounds, such as salts, reagents, or byproducts thereof present in a proteolytically digested sample, can be eluted from the reversed-phase medium prior to the proteolytic polypeptides using an aqueous mobile phase.

In some embodiments, the reversed-phase medium comprises an alkyl-based moiety covalently bound to a solid phase. In some embodiments, the alkyl-based moiety comprises an alkyl carbon functional group having between 1 carbon and 30 carbons, such as any of 4 carbons to 18 carbons, 8 carbons to 18 carbons, or 18 carbons to 30 carbons. In some embodiments, the alkyl-based moiety comprises an alkyl carbon functional group having 30 or fewer carbons, such as any of 25 or fewer carbons, 20 or fewer carbons, 19 or fewer carbons, 18 or fewer carbons, 17 or fewer carbons, 16 or fewer carbons, 15 or fewer carbons, 14 or fewer carbons, 13 or fewer carbons, 12 or fewer carbons, 11 or fewer carbons, 10 or fewer carbons, 9 or fewer carbons, 8 or fewer carbons, 7 or fewer carbons, 6 or fewer carbons, 5 or fewer carbons, 4 or fewer carbons. In some embodiments, the alkyl-based moiety comprises an alkyl carbon functional group comprising 4 carbons, 5 carbons, 6 carbons, 7 carbons, 8 carbons, 9 carbons, 10 carbons, 11 carbons, 12 carbons, 13 carbons, 14 carbons, 15 carbons, 16 carbons, 17 carbons, 18 carbons, 19 carbons, 20 carbons, 21 carbons, 22 carbons, 23 carbons, 24 carbons, 25 carbons, 26 carbons, 27 carbons, 28 carbons, 29 carbons, or 30 carbons. In some embodiments, the alkyl-based moiety comprises an octadecyl carbon functional group (C18) covalently bound to the solid phase. In some embodiments, the alkyl-based moiety comprises an octa carbon functional group (C8) covalently bound to the solid phase. In some embodiments, the carbon alkyl-based moiety comprises a tetra carbon functional group (C4) covalently bound to the solid phase.

In some embodiments, the reversed-phase medium comprises a silica-based material which supports an alkyl-based moiety. In some embodiments, the solid phase comprises a silica material. In some embodiments, the silica material is a silica gel, such as composed of a plurality of silica particles. In some embodiments, the silica-based material is inert. In some embodiments, the silica-based material is base-deactivated. In some embodiments, the silica-based material is an ultra-high purity silica material, such as an ultra-high purity silica gel. In some embodiments, the silica-based material comprises silanol groups that are partially or fully end-capped, such as, for example, with a C1 methyl group. In some embodiments, the silica-based material comprises silanol groups are not end-capped. The components of a silica-based material of a reversed-phase medium may take a diverse array of sizes and shapes. In some embodiments, the silica-based material comprises a plurality of particles, wherein the plurality of particles have an average largest cross-sectional distance (such as a diameter, e.g., as measured by dynamic light scattering) of about 0.5 μm to about 30 μm, such as any of about 1 μm to about 25 μm, about 15 μm to about 25 μm, about 18 μm to about 22 μm. In some embodiments, the silica-based material comprises a plurality of particles, wherein the plurality of particles have an average largest cross-sectional distance (such as a diameter, e.g., as measured by dynamic light scattering) of about any of 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, or 30 μm.

In some embodiments, the silica-based material comprises a plurality of particles, wherein each particle of the plurality of particles comprises an average pore size of about 1 Å to about 500 Å, such as about 50 Å to about 300 Å, or about 100 Å to about 200 Å. In some embodiments, the silica-based material comprises a plurality of particles, wherein each particle of the plurality of particles comprises an average pore size of about any of 5 Å, 10 Å, 20 Å, 25 Å, 30 Å, 35 Å, 40 Å, 45 Å, 50 Å, 60 Å, 70 Å, 80 Å, 90 Å, 100 Å, 110 Å, 120 Å, 130 Å, 140 Å, 150 Å, 160 Å, 170 Å, 180 Å, 190 Å, 200 Å, 225 Å, 250 Å, 275 Å, 300 Å, 325 Å, 350 Å, 375 Å, 400 Å, 425 Å, 450 Å, 475 Å, or 500 Å. In some embodiments, the pore size of the silica-based medium is uniform or substantially uniform. In some embodiments, the pore size of the silica-based medium is heterogeneous. In some embodiments, the pores of the silica-based medium are derivatized with an alkyl-based moiety.

In some embodiments, the reversed-phase medium comprises a hydrophobic polymer material (e.g., RP-S). In some embodiments, the hydrophobic polymer material comprises a phenyl moiety. In some embodiments, the hydrophobic polymer material comprises a reaction product of divinylbenzene. In some embodiments, the hydrophobic polymer material comprises poly(styrene-co-divinylbenzene). In some embodiments the reversed-phase medium comprising a hydrophobic polymer material is in the form a plurality of particles. The components of a hydrophobic polymer material (e.g., RP-S) reversed-phase medium may take a diverse array of sizes and shapes. In some embodiments, the hydrophobic polymer material comprises a plurality of particles, wherein the plurality of particles have an average largest cross-sectional distance (such as a diameter, e.g., as measured by dynamic light scattering) of about 0.5 μm to about 30 μm, such as any of about 1 μm to about 25 μm, about 15 μm to about 25 μm, about 18 μm to about 22 μm. In some embodiments, the hydrophobic polymer material comprises a plurality of particles, wherein the plurality of particles have an average largest cross-sectional distance (such as a diameter, e.g., as measured by dynamic light scattering) of about any of 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, or 30 μm. In some embodiments, the hydrophobic polymer material comprises a plurality of particles, wherein each particle of the plurality of particles comprises an average pore size of about 1 Å to about 500 Å, such as about 50 Å to about 300 Å, or about 100 Å to about 200 Å. In some embodiments, the hydrophobic polymer material comprises a plurality of particles, wherein each particle of the plurality of particles comprises an average pore size of about any of 5 Å, 10 Å, 20 Å, 25 Å, 30 Å, 35 Å, 40 Å, 45 Å, 50 Å, 60 Å, 70 Å, 80 Å, 90 Å, 100 Å, 110 Å, 120 Å, 130 Å, 140 Å, 150 Å, 160 Å, 170 Å, 180 Å, 190 Å, 200 Å, 225 Å, 250 Å, 275 Å, 300 Å, 325 Å, 350 Å, 375 Å, 400 Å, 425 Å, 450 Å, 475 Å, or 500 Å. In some embodiments, the pore size of the hydrophobic polymer material is uniform or substantially uniform. In some embodiments, the pore size of the hydrophobic polymer material is heterogeneous.

The reversed-phase media in the solid phase extraction columns may take a diverse array of forms. In some embodiments, the reversed-phase medium is in the form of a plurality of particles, wherein the solid phase extraction column comprises a packed column. In some embodiments, the solid phase extraction column comprises a surface modification forming the reversed-phase medium. In some embodiments, the solid phase extraction column comprises a monolithic structure. In some instances, the terms medium, media, and resin may be used interchangeably.

In some embodiments, the solid phase extraction column has a column volume of about 1 to about 10 μL, such as any of about 2 μL to about 8 μL, about 3 μL to about 7 μL, or about 4 μL to about 5 μL. In some embodiments, the solid phase extraction column has a column volume of about any of 1 μL, 2 μL, 3 μL, 4 μL, 5 μL, 6 μL, 7 μL, 8 μL, 9 μL, or 10 μL. The reversed-phase media can be referred to as a bed that is compacted within a chromatography column to form a bed volume.

In some embodiments, the reversed-phase medium comprises an alkyl-based moiety comprising an octadecyl carbon functional group (C18) covalently bound to a silica solid phase, wherein the silica solid phase comprises a plurality of particles having an average largest cross-sectional distance (such as a diameter, e.g., as measured by dynamic light scattering) of about 20 μm, and wherein the each particle of the plurality of particles comprises an average pore size of about 150 Å, and wherein the pores are derivatized with the alkyl-based moiety. In some embodiments, the solid phase extraction column comprising the reversed-phase medium has a column volume of about 5 μL. In some embodiments, the solid phase extraction column is an AssayMap 5 μL C18 cartridge (catalog no. 5190-6532; Agilent Technologies).

In some embodiments, the reversed-phase medium comprises an underivitized polystyrene divinylbenzene hydrophobic reversed-phase resin, wherein the polystyrene divinylbenzene hydrophobic reversed-phase resin comprises a plurality of particles having an average largest cross-sectional distance (such as a diameter, e.g., as measured by dynamic light scattering) of about 20 μm, and wherein each particle of the plurality of particles comprises an average pore size of about 100 Å. In some embodiments, the solid phase extraction column comprising the reversed-phase medium has a column volume of about 5 μL. In some embodiments, the solid phase extraction column is an AssayMAP 5 μL Reversed Phase (RP-S) cartridge (catalog no. G5496-60033; Agilent Technologies).

### V. Additional Aspects of the Methods for Processing

The steps involved with the taught methods include the steps of loading a proteolytically digest sample onto a solid phase extraction column and washing proteolytic polypeptides associated with the reversed-phase medium of the solid phase extraction column. In some embodiments, these steps comprise the use of solutions to facilitate the performance of said step. In some embodiments, the proteolytically digested sample for loading onto a solid phase extraction column has a pH of less than 3, such as for use with a C18 or RP-S solid phase extraction column. In some embodiments, the proteolytically digested sample for loading onto a solid phase extraction column has a pH of greater than 10, such as for use with a RP-S solid phase extraction column. In some embodiments, the proteolytically digested sample comprises a loading solution, such as water with an acid, such as TFA, wherein the final concentration of TFA in the proteolytically digested sample is 1% or less. In some embodiments, the loading solution comprises 0.1% TFA in water. In some embodiments, the wash buffer is an aqueous solution (such as water) with 1% or less of an acid, such as 0.1% TFA in water. In some embodiments, the components of the loading solutions and wash buffers are HPLC-grade.

In certain aspects, the methods for processing a proteolytically digested sample to produce a processed sample suitable for use in a LC-MS analysis provided herein comprise further steps for the production of the processed sample.

In some embodiments, the method comprises any one or more of: (a) a solid phase extraction column priming step; (b) a solid phase extraction column equilibration step; (c) a sample loading step; (d) a wash step; or (e) an elution step. In some embodiments, the priming step comprises passing an amount (such as 1 column volume to about 20 column volumes) of a solution comprising at least 25% organic (such as 50% ACN with 0.1% TFA) through the solid phase extraction column. In some embodiments, the equilibration step comprises passing an amount (such as 1 column volume to about 50 column volumes) of 0.1% TFA in water through the solid phase extraction column. In some embodiments, the elution step comprises passing an amount (such as 1 column volume to about 50 column volumes) of a solution comprising at least about 50% organic (such as 50% ACN with 0.1% TFA) through the solid phase extraction column. In some embodiments, the method comprises one or more steps according to a manufacturer's instructions, such as for AssayMap 5 μL C18 cartridge (catalog no. 5190-6532; Agilent Technologies) or AssayMAP 5 μL Reversed Phase (RP-S) cartridge (catalog no. G5496-60033; Agilent Technologies).

In some embodiments, the method provided herein produces a processed sample suitable for use in a LC-MS technique. In some embodiments, the processed sample has a yield of at least about 70%, such as at least about any of 75%, 80%, 85%, 90%, or 95%, relative to the total polypeptide content of the proteolytically digested sample. In some embodiments, the processed sample has a glycopeptide yield (such as assessed from one or more, including all, glycopeptides in a proteolytically digested sample) of at least about 70%, such as at least about any of 75%, 80%, 85%, 90%, or 95%, relative to the total glycopolypeptide content of the proteolytically digested sample.

In some embodiments, wherein the method for processing a proteolytically digested sample is performed in replicate (e.g., two or more aliquots of a proteolytically digested sample are processed using the same method for processing), the resulting coefficient of variation (CV) of a peak feature, such as peak area of the measured polypeptide, e.g., glycopolypeptide, is about 15% or less, such as about any of 14% or less, 13% or less, 12% or less, 11% or less, 10% or less, 9% or less, 8% or less, 7% or less, 6% or less, 5% or less, 4% or less, 3% or less, 2% or less, or 1% or less.

In some embodiments, the proteolytically digested sample and the processed sample produced therefrom using the methods described herein comprises at least one glycopeptide. In some embodiments, the glycopeptide comprises one or more, including 1, 2, 3, 4, or 5, sialic acid moieties.

In some embodiments, the method comprises generating a unity plot to compare different processing methods. Unity plots are known in the art. In a unity plot, a null effect between the two plotted conditions (e.g., processing methods) is indicated when plotted points run along the unity line. When the data points of a unity plot are skewed above or below the unity line, then biasing can be identified to indicate a preferred condition. For example, biasing above the unity line indicates improved results with the y-axis condition, and biasing below the unity line indicates improved results with the x-axis condition.

### D2. Methods for Proteolytically Digesting a Biological Sample Comprising a Glycoprotein

In certain aspects, provided herein are methods of proteolytically digesting a biological sample comprising a glycoprotein, the methods comprising subjecting the biological sample to a thermal denaturation technique. Proteases are enzymes that cleave polypeptides at, generally, specific cleavage motifs. For example, trypsin is a serine protease that generally cleaves polypeptides at the carboxyl side (C-terminal side) of lysine and arginine residues. A glycan of a glycopeptide may present a steric hindrance to a protease, thereby inhibiting complete protease digestion of a biological sample comprising a glycoprotein. Without being bound to this theory, it is believed that the methods taught herein improve polypeptide unfolding, such as linearization, and provide protease access to cleavage sites thereby providing methods for more complete proteolytic digestion of glycoproteins.

In some aspects, provided is a method comprising subjecting a biological sample to a thermal denaturation technique to produce a denatured sample.

In other aspects, provided is a method comprising subjecting a biological sample to a thermal denaturation technique to produce a denatured sample followed by a proteolytic digestion technique to produce a proteolytically digested sample comprising a proteolytic glycopeptide. In some embodiments, the method comprises quenching one or more proteases used in a proteolytic digestion technique prior to a downstream technique, such as LC-MS.

In other aspects, provided herein is a method comprising: subjecting a biological sample to a thermal denaturation technique to produce a denatured sample; subjecting the denatured sample to a reduction technique to produce a reduced sample; subjecting the reduced sample to an alkylation technique to produce an alkylated sample; and subjecting the alkylated sample to a proteolytic digestion technique to produce a proteolytically digested sample comprising the proteolytic glycopeptide. In some embodiments, the method comprises quenching an alkylating agent used in the alkylation technique prior to subjecting an alkylated sample to a proteolytic digestion technique. In some embodiments, the method comprises quenching one or more proteases used in a proteolytic digestion technique prior to a downstream technique, such as LC-MS.

In some embodiments, the proteolytic digestion methods described herein produce a proteolytic digestion sample, including one comprising a proteolytic glycopeptide, having a digestion completion rate of at least about 70%, such as at least about any of 75%, 80%, 85%, 90%, 91%, 92%, 93%, 94%, 95%, 96%, 97%, 98%, or 99%. In some embodiments, the proteolytic digestion methods described herein produce a proteolytic digestion sample, including one comprising a proteolytic glycopeptide, wherein the sample volume loss is 10% or less, such as 9% or less, 8% or less, 7% or less, 6% or less, 5% or less, 4% or less, 3% or less, 2% or less, or 1% or less, based on all volumes added in producing the proteolytic digestion sample.

In the following sections, additional description of the various aspects of the proteolytic digestion techniques is provided. Such description in a modular fashion is not intended to limit the scope of the disclosure, and based on the teachings provided herein one of ordinary skill in the art will readily appreciate that certain modules can be integrated, at least in part. The section heading used herein are for organizational purposes only and are not to be construed as limiting the subject matter described.

### I. Thermal Denaturation Techniques

In certain aspects, the methods provided herein comprise performing a thermal denaturation technique. Thermal denaturation techniques, generally speaking, change certain polypeptides conformational structures, such as by unfolding and/or linearizing a polypeptide, to enable protease access to cleavage sites. Thermal denaturation techniques described herein comprise subjecting a sample, or a derivative thereof (e.g., a sample diluted with a buffer), to a thermal treatment of about 60° C. to about 100° C. for thermal denaturation incubation time of at least about 1 minute. In some embodiments, the thermal denaturation technique is not performed concurrently with a chemical denaturation technique, such as using high concentrations of denaturing agent, e.g., 6M urea. In some embodiments, the method does not include use of a chemical denaturation technique.

In some embodiments, the thermal denaturation incubation time is performed at a temperature of about 60° C. to about 100° C., such as any of about 70° C. to about 100° C., about 80° C. to about 100° C., about 90° C. to about 100° C., about 95° C. to about 100° C., or about 85° C. to about 95° C. In some embodiments, the thermal denaturation incubation time is performed at a temperature of at least about 60° C., such as at least about any of 65° C., 70° C., 75° C., 80° C., 85° C., 90° C., 95° C., or 100° C. In some embodiments, the thermal denaturation incubation time is performed at a temperature of about 100° C. or less, such as about any of 95° C. or less, 90° C. or less, 85° C. or less, 80° C. or less, 75° C. or less, 70° C. or less, 65° C. or less, 60° C. or less. In some embodiments, the thermal denaturation incubation time is performed at a temperature of about any of 60° C., 65° C., 70° C., 75° C., 80° C., 85° C., 90° C., 91° C., 92° C., 93° C., 94° C., 95° C., 96° C., 97° C., 98° C., 99° C., or 100° C.

In some embodiments, the thermal denaturation incubation time is about 1 minute to about 15 minutes, such as any of about 1 minute to about 5 minutes, about 1 minute to about 10 minutes, about 2.5 minutes to about 7.5 minutes, or about 5 minutes to about 15 minutes. In some embodiments, the thermal denaturation incubation time is at least about 1 minute, such as at least about any of 1.5 minutes, 2 minutes, 2.5 minutes, 3 minutes, 3.5 minutes, 4 minutes, 4.5 minutes, 5 minutes, 5.5 minutes, 6 minutes, 6.5 minutes, 7 minutes, 7.5 minutes, 8 minutes, 8.5 minutes, 9 minutes, 9.5 minutes, 10 minutes, 11 minutes, 12 minutes, 13 minutes, 14 minutes, or 15 minutes. In some embodiments, the thermal denaturation incubation time is about 15 minutes or less, such as about any of 14 minutes or less, 13 minutes or less, 12 minutes or less, 11 minutes or less, 10 minutes or less, 9.5 minutes or less, 9 minutes or less, 8.5 minutes or less, 8 minutes or less, 7.5 minutes or less, 7 minutes or less, 6.5 minutes or less, 6 minutes or less, 5.5 minutes or less, 5 minutes or less, 4.5 minutes or less, 4 minutes or less, 3.5 minutes or less, 3 minutes or less, 2.5 minutes or less, 2 minutes or less, 1.5 minutes or less, or 1 minute or less. In some embodiments, the thermal denaturation incubation time is about any of 1 minute, 1.5 minutes, 2 minutes, 2.5 minutes, 3 minutes, 3.5 minutes, 4 minutes, 4.5 minutes, 5 minutes, 5.5 minutes, 6 minutes, 6.5 minutes, 7 minutes, 7.5 minutes, 8 minutes, 8.5 minutes, 9 minutes, 9.5 minutes, 10 minutes, 11 minutes, 12 minutes, 13 minutes, 14 minutes, or 15 minutes.

In some embodiments, the thermal denaturation technique comprises a thermal denaturation incubation time of about 1 minute to about 15 minutes, such as about any of 1 minute, 2 minutes, 3 minutes, 4 minutes, 5 minutes, 6 minutes, 7 minutes, 8 minutes, 9 minutes 10 minutes, 11 minutes, 12 minutes, 13 minutes, 14 minutes, or 15 minutes, wherein the thermal denaturation incubation is performed at a temperature of about 60° C. to about 100° C., such about any of 60° C., 65° C., 70° C., 75° C., 80° C., 85° C., 90° C., 91° C., 92° C., 93° C., 94° C., 95° C., 96° C., 97° C., 98° C., 99° C., or 100° C.

The thermal denaturation incubation temperature can be controlled by numerous techniques and combinations thereof. In some embodiments, the thermal denaturation incubation temperature is controlled by a water bath. In some embodiments, the thermal denaturation incubation temperature is controlled by a heat block. In some embodiments, the thermal denaturation incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the thermal denaturation incubation time temperature are controlled by a technique described herein, such as cooling at room temperature or via the thermocycler.

In some embodiments, the thermal denaturation technique comprises subjecting a sample, or a derivative thereof, e.g., a sample diluted in a buffer, to a thermal cycle. In some embodiments, the thermal cycle comprises subjecting the sample, or a derivative thereof, to one or more of: (a) a block starting temperature (b) block set temperature (the temperature for the thermal denaturation incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 60° C. to about 100° C., including about any of 60° C., 65° C., 70° C., 75° C., 80° C., 85° C., 90° C., 91° C., 92° C., 93° C., 94° C., 95° C., 96° C., 97° C., 98° C., 99° C., or 100° C. In some embodiments, the thermal cycle comprises: (a) a set block temperature of about 60° C. to about 100° C., including about any of 60° C., 65° C., 70° C., 75° C., 80° C., 85° C., 90° C., 91° C., 92° C., 93° C., 94° C., 95° C., 96° C., 97° C., 98° C., 99° C., or 100° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 50° C., such as any of 15° C. to about 25° C., about 20° C. to about 30° C., or about 20° C. to about 25° C., including about any of 20° C., 21° C., 22° C., 23° C., 24° C., 25° C., (b) a set block temperature of about 60° C. to about 100° C., such about any of 60° C., 65° C., 70° C., 75° C., 80° C., 85° C., 90° C., 91° C., 92° C., 93° C., 94° C., 95° C., 96° C., 97° C., 98° C., 99° C., or 100° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C. In some embodiments, the lid temperature during the thermal cycle may be the same respective temperature of the block during the thermal cycle or a temperature greater than the temperature of the block during the thermal cycle. In some embodiments, the lid temperature during the thermal cycle may be the same respective temperature of the block during the thermal cycle or a temperature greater than the temperature of the block during the thermal cycle.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

In some embodiments, the method further comprises admixing an amount of a biological sample a buffer prior to the thermal denaturation technique (e.g., the buffered sample is subjected to a thermal denaturation technique described herein). In some embodiment, the amount (as assessed based on the final concentration in the sample containing solution containing solution) of the buffer is about 1 mM to about 100 mM, such as any of about 20 mM to about 80 mM, about 30 mM to about 70 mM, or about 40 mM to about 60 mM. In some embodiment, the amount of the buffer is about any of 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some embodiments, the buffer is selected from the group consisting of ammonium bicarbonate, ammonium acetate, ammonium formate, triethylammonium bicarbonate, and Tris-HCl, or any combination thereof.

In some embodiments, the method further comprises determining the protein concentration in a biological sample or a derivative thereof.

### II. Reduction Techniques

In certain aspects, the methods provided herein comprise performing a reduction technique. In some embodiments, the reduction technique is performed on a sample, or a derivative thereof, following thermal denaturation. Reduction techniques, generally speaking, reduce (e.g., cleave) disulfide linkages between cysteine residues of one or more polypeptides to reduce the presence of polypeptide conformations that inhibit or prevent protease cleavage of the one or more polypeptides. Reduction techniques described herein comprise subjecting a sample, or a derivative thereof (e.g., a denatured sample), to an amount of a reducing agent and incubating for a reducing incubation time performed at a temperature or range thereof.

In some embodiments, the reducing agent is dithiothreitol (DTT), tris(2-carboxyethyl) phosphine (TCEP), beta-mercaptoethanol (BME), or a cysteine, or any mixture thereof.

In some embodiments, the amount (as assessed based on the final concentration in the sample containing solution containing solution) of a reducing agent, e.g., DTT, used in a reduction technique is about 1 mM to about 100 mM, such as any of about 1 mM to about 40 mM, about 1 mM to about 30 mM, about 5 mM to about 25 mM, about 10 mM, to about 20 mM, 20 mM to about 80 mM, about 30 mM to about 70 mM, or about 40 mM to about 60 mM. In some embodiments, the amount of reducing agent used in a reduction technique is at least about 1 mM, such as at least about any of 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some embodiments, the amount of reducing agent used in a reduction technique is about 100 mM or less, such as about any of 95 mM or less, 90 mM or less, 85 mM or less, 80 mM or less, 75 mM or less, 70 mM or less, 65 mM or less, 60 mM or less, 55 mM or less, 50 mM or less, 45 mM or less, 40 mM or less, 35 mM or less, 30 mM or less, 25 mM or less, 20 mM or less, 19 or less, 18 or less, 17 or less, 16 or less, 15 or less, 14 or less, 13 or less, 12 or less, 11 or less, 10 or less, 9 or less, 8 or less, 7 or less, 6 or less, 5 or less, 4 or less, 3 or less, 2 or less, or 1 or less. In some embodiments, the amount of reducing agent used in a reduction technique is about any of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In some embodiments, the reduction incubation time is about 10 minutes to about 120 minutes, such as any of about 30 minutes to about 60 minutes, about 40 minutes to about 60 minutes, about 45 minutes to about 55 minutes. In some embodiments, the reduction incubation time is at least about 20 minutes, such as at least about any of 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes. In some embodiments, the reduction incubation time is about 120 minutes or less, such as about any of 115 minutes or less, 110 minutes or less, 105 minutes or less, 100 minutes or less, 95 minutes or less, 90 minutes or less, 85 minutes or less, 80 minutes or less, 75 minutes or less, 70 minutes or less, 65 minutes or less, 60 minutes or less, 55 minutes or less, 50 minutes or less, 45 minutes or less, 40 minutes or less, 35 minutes or less, 30 minutes or less, or 25 minutes or less. In some embodiments, the reduction incubation time is about any of 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes.

In some embodiments, the reduction incubation time is performed at a temperature of about 20° C. to about 100° C., such as any of about 40° C. to about 80° C., about 50° C. to about 70° C., about 50° C. to about 60° C., about 55° C. to about 65° C., or about 60° C. to about 70° C. In some embodiments, the reduction incubation time is performed at a temperature of at least about 20° C., such as at least about any of 25° C., 30° C., 35° C., 40° C., 45° C., 50° C., 55° C., 60° C., 65° C., 70° C., 75° C., 80° C., 85° C., 90° C., or 95° C. In some embodiments, the reduction incubation time is performed at a temperature of about 95° C. or less, such as about any of 90° C. or less, 85° C. or less, 80° C. or less, 75° C. or less, 70° C. or less, 65° C. or less, 60° C. or less, 55° C. or less, 50° C. or less, 45° C. or less, 40° C. or less, 35° C. or less, 30° C. or less, or 25° C. or less. In some embodiments, the reduction incubation time is performed at a temperature of about any of 20° C., 25° C., 30° C., 35° C., 40° C., 45° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 75° C., 80° C., 85° C., 90° C., or 95° C. In some embodiments, the reduction incubation time is performed at a room temperature.

In some embodiments, the reduction technique comprises a reduction incubation time of about 30 minutes to about 70 minutes, such as about any of 35 minutes, 40 minutes, 45 minutes, 50 minutes, 55 minutes, 60 minutes, or 65 minutes, wherein the reduction incubation time is performed at a temperature of about 50° C. to about 70° C., such about any of 55° C., 60° C., or 65° C. In some embodiments, the reduction technique comprises use of an amount (as assessed based on the final concentration in the sample containing solution) of a reducing agent, e.g., DTT, of about 5 mM to about 25 mM, such as any of about 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, or 24 mM, and a reduction incubation time of about 30 minutes to about 70 minutes, such as about any of 35 minutes, 40 minutes, 45 minutes, 50 minutes, 55 minutes, 60 minutes, or 65 minutes, wherein the reduction incubation time is performed at a temperature of about 50° C. to about 70° C., such about any of 55° C., 60° C., or 65° C.

The reduction incubation temperature can be controlled by numerous techniques and combinations thereof. In some embodiments, the reduction incubation temperature is controlled by an ambient temperature, such as room temperature. In some embodiments, the reduction incubation temperature is controlled by a water bath. In some embodiments, the reduction incubation temperature is controlled by a heat block. In some embodiments, the reduction incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the reduction incubation time temperature are controlled by a technique described herein, such as cooling at room temperature or a ramp rate.

In some embodiments, the reduction technique comprises subjecting a sample, or a derivative thereof, e.g., a denatured sample, to a thermal cycle. In some embodiments, the thermal cycle comprises subjecting the sample, or a derivative thereof, to one or more of: (a) a block starting temperature (b) block set temperature (the temperature for the reduction incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 20° C. to about 100° C., such as any of 40° C. to about 80° C., about 50° C. to about 70° C., about 50° C. to about 60° C., about 55° C. to about 65° C., or about 60° C. to about 70° C., including about any of 50° C., 55° C., 60° C., 65° C., or 70° C. In some embodiments, the thermal cycle comprises: (a) a set block temperature of about 20° C. to about 100° C., such as any of about 40° C. to about 80° C., about 50° C. to about 70° C., about 50° C. to about 60° C., about 55° C. to about 65° C., or about 60° C. to about 70° C., including about any of 50° C., 55° C., 60° C., 65° C., or 70° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 60° C., such as any of about 15° C. to about 50° C., about 20° C. to about 40° C., about 20° C. to about 30° C., or about 20° C. to about 25° C., (b) a set block temperature of about 20° C. to about 100° C., such as any of about 40° C. to about 80° C., about 50° C. to about 70° C., about 50° C. to about 60° C., about 55° C. to about 65° C., or about 60° C. to about 70° C., including about any of 50° C., 55° C., 60° C., 65° C., or 70° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

In some embodiments, the reduction technique described herein is completed simultaneously with a thermal denaturation step. For example, the combined thermal denaturation technique and reduction technique comprises adding a reducing agent to a sample, or a derivative thereof, and then subjecting the sample, or the derivative thereof, to a temperature of about 60° C. to about 100° C., such about any of 60° C., 65° C., 70° C., 75° C., 80° C., 85° C., 90° C., 91° C., 92° C., 93° C., 94° C., 95° C., 96° C., 97° C., 98° C., 99° C., or 100° C., for an incubation time of at least about 1 minute, such as at least about any of 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes, or 60 minutes. In some embodiments, the combined thermal denaturation technique and reduction technique comprises adding a reducing agent to a sample, or a derivative thereof, and then subjecting the sample, or the derivative thereof, to a temperature of about 90° C. to about 100° C., for an incubation time of about 40 minutes to about 60 minutes, including 50 minutes.

### III. Alkylation Techniques

In certain aspects, the methods provided herein comprise performing an alkylation technique. In some embodiments, the alkylation technique is performed on a sample, or a derivative thereof, following the performance of a reduction technique. Alkylation techniques, generally speaking, prevent the reformation of one or more disulfide linkages between, e.g., cysteine residues of one or more polypeptides. This is done by, e.g., the addition of an acetamide moiety to the sulfur of a cysteine residue thereby producing an alkylated polypeptide. Alkylation techniques may reduce the presence of polypeptide conformations that inhibit or prevent protease cleavage of the one or more polypeptides. Alkylation techniques described herein comprise subjecting a sample, or a derivative thereof (e.g., a reduced sample), to an amount of an alkylating agent and incubating for an alkylation incubation time performed at a temperature or range thereof. In some embodiments, the method comprises subjecting a denatured sample to a reduction technique followed by an alkylation technique prior to performing a proteolytic digestion technique.

In some embodiments, the alkylating agent is iodoacetamide (IAA), 2-chloroacetamide, an acetamide salt, or any mixture thereof.

In some embodiments, the amount (as assessed based on the final concentration in the sample containing solution) of an alkylating agent, e.g., IAA, used in an alkylation technique is about 10 mM to about 100 mM, such as any of about 10 mM to about 50 mM, about 20 mM to about 40 mM, about 20 mM to about 36 mM, about 15 mM to about 25 mM, about 20 mM to about 25 mM, about 20 mM to about 80 mM, about 30 mM to about 70 mM, or about 40 mM to about 60 mM. In some embodiments, the amount of an alkylating agent used in an alkylation technique is at least about 10 mM, such as at least about any of 15 mM, 16 mM, 17 mM, 18 mM, 19 mM, 20 mM, 21 mM, 22 mM, 23 mM, 24 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some embodiments, the amount of an alkylating agent used in an alkylation technique is about 100 mM or less, such as about any of 95 mM or less, 90 mM or less, 85 mM or less, 80 mM or less, 75 mM or less, 70 mM or less, 65 mM or less, 60 mM or less, 55 mM or less, 50 mM or less, 45 mM or less, 40 mM or less, 35 mM or less, 30 mM or less, 25 mM or less, 24 mM or less, 23 mM or less, 22 mM or less, 21 mM or less, 20 mM or less, 19 mM or less, 18 mM or less, 17 mM or less, 16 mM or less, 15 mM or less, or 10 mM or less. In some embodiments, the amount of an alkylating agent used in an alkylation technique is about any of 10 mM, 15 mM, 20 mM, 20.5 mM, 21 mM, 21.5 mM, 22 mM, 22.5 mM, 23 mM, 23.5 mM, 24 mM, 24.5 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In some embodiments, the alkylation incubation time is about 5 minutes to about 60 minutes, such as any of about 10 minutes to about 50 minutes, about 20 minutes to about 40 minutes, about 25 minutes to about 35 minutes. In some embodiments, the alkylation incubation time is at least about 5 minutes, such as at least about any of 10 minutes, 15 minutes, 20 minutes, 25 minutes, 30 minutes, 35 minutes, 40 minutes, 45 minutes, 50 minutes, 55 minutes, or 60 minutes. In some embodiments, the alkylation incubation time is about 60 minutes or less, such as about any of 55 minutes or less, 50 minutes or less, 45 minutes or less, 40 minutes or less, 35 minutes or less, 30 minutes or less, 25 minutes or less, 20 minutes or less, 15 minutes or less, 10 minutes or less, or 5 minutes or less. In some embodiments, the alkylation incubation time is about any of 5 minutes, 10 minutes, 15 minutes, 20 minutes, 25 minutes, 30 minutes, 35 minutes, 40 minutes, 45 minutes, 50 minutes, 55 minutes, or 60 minutes.

In some embodiments, the alkylation incubation time is performed at a temperature of about 15° C. to about 100° C., such as any of about 15° C. to about 80° C., about 15° C. to about 60° C., about 15° C. to about 35° C., about 20° C. to about 30° C., or about 20° C. to about 25° C. In some embodiments, the alkylation incubation time is performed at a temperature of at least about 15° C., such as at least about any of 20° C., 21° C., 22° C., 23° C., 24° C., 25° C., 30° C., 35° C., 40° C., 45° C., 50° C., 55° C., 60° C., 65° C., 70° C., 75° C., 80° C., 85° C., 90° C., or 95° C. In some embodiments, the alkylation incubation time is performed at a temperature of about 95° C. or less, such as about any of 90° C. or less, 85° C. or less, 80° C. or less, 75° C. or less, 70° C. or less, 65° C. or less, 60° C. or less, 55° C. or less, 50° C. or less, 45° C. or less, 40° C. or less, 35° C. or less, 30° C. or less, 25° C. or less, 24° C. or less, 23° C. or less, 22° C. or less, 21° C. or less, or 20° C. or less. In some embodiments, the alkylation incubation time is performed at a temperature of about any of 15° C., 16° C., 17° C., 18° C., 19° C., 20° C., 21° C., 22° C., 23° C., 24° C., 25° C., 30° C., 35° C., 40° C., 45° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 75° C., 80° C., 85° C., 90° C., or 95° C. In some embodiments, the alkylation incubation time is performed at a room temperature.

In some embodiments, the alkylation technique comprises an alkylation incubation time of about 5 minutes to about 60 minutes, such as about any of 15 minutes, 20 minutes, 25 minutes, 30 minutes, 35 minutes, or 40 minutes, wherein the alkylation incubation time is performed at a temperature of about 15° C. to about 30° C., such about any of 20° C., 21° C., 22° C., 23° C., 24° C., 25° C. In some embodiments, the alkylation technique comprises use of an amount (containing solution based on the final concentration in the sample) of an alkylating agent, e.g., IAA, of about 15 mM to about 40 mM, such as any of about 20 mM, 20.5 mM, 21 mM, 21.5 mM, 22 mM, 22.5 mM, 23 mM, 23.5 mM, 24 mM, 24.5 mM, 25 mM, 25.5 mM, 26 mM, 26.5 mM, 27 mM, 27.5 mM, 28 mM, 28.5 mM, 29 mM, 29.5 mM, 30 mM, 30.5 mM, 31 mM, 31.5 mM, 32 mM, 32.5 mM, 33 mM, 33.5 mM, 34 mM, 34.5 mM, 35 mM, 35.5 mM, 36 mM, 36.5 mM, 37 mM, 37.5 mM, 38 mM, and an alkylation incubation time of about 5 minutes to about 60 minutes, such as about any of 15 minutes, 20 minutes, 25 minutes, 30 minutes, 35 minutes, or 40 minutes, wherein the alkylation incubation time is performed at a temperature of about 15° C. to about 30° C., such about any of 20° C., 21° C., 22° C., 23° C., 24° C., or 25° C.

The alkylation incubation temperature can be controlled by numerous techniques and combinations thereof. In some embodiments, the alkylation incubation temperature is controlled by an ambient temperature, such as room temperature. In some embodiments, the alkylation incubation temperature is controlled by a water bath. In some embodiments, the alkylation incubation temperature is controlled by a heat block. In some embodiments, the alkylation incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the alkylation incubation time temperature are controlled by a technique described herein, such as cooling at room temperature or a ramp rate.

In some embodiments, the alkylation technique comprises subjecting a sample, or a derivative thereof, e.g., a denatured sample, to a thermal cycle. In some embodiments, the thermal cycle comprises subjecting the sample, or a derivative thereof, to one or more of: (a) a block starting temperature (b) block set temperature (the temperature for the alkylation incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., or 25° C. In some embodiments, the thermal cycle comprises: (a) a set block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., 25° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., 25° C., (b) a set block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., 25° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C.

In some embodiments, the alkylation technique further comprises quenching the alkylating agent comprising use of a neutralizing agent. In some embodiments, the neutralizing agent is a reducing agent. In some embodiments, the reducing agent is dithiothreitol (DTT), tris(2-carboxyethyl) phosphine (TCEP), beta-mercaptoethanol (BME), or a cysteine, or any mixture thereof. In some embodiments, the neutralizing agent is added in an amount to fully quench the amount of the alkylating agent, such as in an amount greater than or equal to a molar amount of an active moiety of the alkylating agent. In some embodiments, the amount (as assessed based on the final concentration in the sample containing solution) of the neutralizing agent is about 1 mM to about 100 mM.

In some embodiments, the alkylation technique, in whole or in part, is performed substantially in a low light condition. In some embodiments, the alkylation incubation time is performed in a low light condition. In some embodiments, the low light condition is in the dark or a location substantially devoid of sunlight and/or room lighting, such as in a desk drawer. In some embodiments, the low light condition is a filtered light, such as red light.

In some embodiments, the alkylating agent is sourced from a stock solution. In some embodiments, the stock solution is prepared within about 1 hour, such as within about any of 50 minutes, 40 minutes, 30 minutes, 20 minutes, or 10 minutes, of use.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

### IV. Proteolytic Digestion Techniques

In certain aspects, the methods provided herein comprise performing a proteolytic digestion technique. In some embodiments, the proteolytic digestion technique is performed on a sample, or a derivative thereof, following thermal denaturation and/or any additional steps intended to expose protease cleavage sites. Proteolytic digestion techniques, generally speaking, cleave polypeptides at known cleavage sites. For example, trypsin is a serine protease that generally cleaves polypeptides at the carboxyl side (C-terminal side) of lysine and arginine residues. Certain exceptions apply to the cleavage pattern of trypsin, such as due to proximity of a proline residue and/or a post-translational modification causing steric hindrance relative to the cleavage site. Proteolytic digestion techniques described herein comprise subjecting a sample, or a derivative thereof (e.g., a denatured sample or an alkylated sample, including an alkylated sample subjected to a reduction technique prior to an alkylation technique), to an amount of one or more proteases and incubating for a digestion incubation time performed at a temperature or range thereof.

In some embodiments, each of the one or more proteases is trypsin, LysC, LysN, AspN, GluC, ArgC, IdeS, IdeZ, PNGase F, thermolysin, pepsin, elastase, TEV, or Factor Xa, or any mixture thereof. In some embodiments, wherein two or more proteases are used, the weight ratio between a first protease and a second protease is about 1:10 to about 10:1, such as about any of about 1:9, 1:8, 1:7: 1:6, 1:5, 1:4, 1:3, 1:2, or 1:1. In some embodiments, the one or more proteases is trypsin. In some embodiments, the one or more proteases is a mixture of trypsin and LysC, such as in a weight ratio of about 1:1. In some embodiments, the one or more proteases is selected based on the type and/or characteristic of a biological sample used in the methods herein. In some embodiments, the biological sample is a plasma sample, wherein the one or more proteases is trypsin and Lys-C, such as in a weight ratio of about 1:1. In some embodiments, the biological sample is a serum sample, wherein the one or more proteases is trypsin. In some embodiments, the protease is a modified protease, such as comprising a modification to prevent or inhibit self-proteolysis. In some embodiments, the modified protease is a modified trypsin, such as a methylated and/or an acetylated trypsin. In some embodiments, the modified trypsin is a tosyl phenylalanyl chloromethyl ketone (TPCK)-treated trypsin.

In some embodiments, the amount of a protease, e.g., trypsin or LysC, used in a proteolytic digestion technique is based on a weight ratio relative to the polypeptide content of a sample, or a derivative thereof, (i.e., weight of a protease: weight of polypeptide content) of about 1:200 to about 1:10, such as any of about 1:100 to about 1:10, about 1:50 to about 1:10, about 1:40 to about 1:20, about 1:50 to about 1:30, about 1:45 to about 1:35, about 1:20 to about 1:40, about 1:30 to about 1:10, or about 1:25 to about 1:15. In some embodiments, the amount of a protease used in a proteolytic digestion technique is at least about 1:200, such as at least about any of 1:190, 1:180, 1:170, 1:160, 1:150, 1:140, 1:130, 1:120, 1:110, 1:100, 1:95, 1:90, 1:85, 1:80, 1:75, 1:70, 1:65, 1:60, 1:55, 1:50, 1:45, 1:40, 1:35, 1:30, 1:25, 1:20, 1:15, or 1:10. In some embodiments, the amount of a protease used in a proteolytic digestion technique is about 1:10 or less, such as about any of 1:15 or less, 1:20 or less, 1:25 or less, 1:30 or less, 1:35 or less, 1:40 or less, 1:45 or less, 1:50 or less, 1:55 or less, 1:60 or less, 1:65 or less, 1:70 or less, 1:75 or less, 1:80 or less, 1:85 or less, 1:90 or less, 1:95 or less, 1:100 or less, 1:110 or less, 1:120 or less, 1:130 or less, 1:140 or less, 1:150 or less, 1:160 or less, 1:170 or less, 1:180 or less, 1:190 or less, or 1:200 or less. In some embodiments, the amount of a protease used in a proteolytic digestion technique is about any of 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, 1:110, 1:120, 1:130, 1:140, 1:150, 1:160, 1:170, 1:180, 1:190, or 1:200. In some embodiments, the proteolytic digestion technique comprises the use of two or more proteases, such as a combination of trypsin and LysC, and in such embodiments, the amount of each protease (such as described above) can be summed to a total amount of proteases used in a proteolytic digestion technique.

In some embodiments, the proteolytic digestion incubation time is about 20 minutes to about 36 hours, such as any of about 1 hour to about 18 hours, about 5 hours to about 24 hours, about 12 hours to about 24 hours, about 16 hours to about 20 hours, or about 12 hours to about 36 hours. In some embodiments, the proteolytic digestion incubation time is about 36 hours or less, such as about any of 32 hours or less, 30 hours or less, 28 hours or less, 26 hours or less, 24 hours or less, 22 hours or less, 20 hours or less, 19 hours or less, 18 hours or less, 17 hours or less, 16 hours or less, 15 hours or less, 14 hours or less, 13 hours or less, 12 hours or less, 11 hours or less, 10 hours or less, 9 hours or less, 8 hours or less, 7 hours or less, 6 hours or less, 5 hours or less, 4 hours or less, 3 hours or less, 2 hours or less, or 1 hours or less. In some embodiments, the proteolytic digestion incubation time is at least about 20 minutes, such as at least about any of 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 22 hours, 24 hours, 26 hours, 28 hours, 30 hours, 32 hours, 34 hours, or 36 hours. In some embodiments, the proteolytic digestion incubation time is about any of 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 22 hours, 24 hours, 26 hours, 28 hours, 30 hours, 32 hours, 34 hours, or 36 hours.

In some embodiments, the digestion incubation time is performed at a temperature of about 20° C. to about 60° C., such as any of about 20° C. to about 25° C., about 20° C. to about 30° C., about 25° C. to about 40° C., about 35° C. to about 40° C., or about 35° C. to about 50° C. In some embodiments, the digestion incubation time is performed at a temperature of at least about 20° C., such as at least about any of 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 42° C., 44° C., 46° C., 48° C., 50° C., 52° C., 54° C., 56° C., 58° C., or 60° C. In some embodiments, the digestion incubation time is performed at a temperature of about 60° C. or less, such about any of 58° C. or less, 56° C. or less, 54° C. or less, 52° C. or less, 50° C. or less, 48° C. or less, 46° C. or less, 44° C. or less, 42° C. or less, 40° C. or less, 39° C. or less, 38° C. or less, 37° C. or less, 36° C. or less, 35° C. or less, 34° C. or less, 33° C. or less, 32° C. or less, 31° C. or less, 30° C. or less, 29° C. or less, 28° C. or less, 27° C. or less, 26° C. or less, 25° C. or less, 24° C. or less, 23° C. or less, 22° C. or less, 21° C. or less, or 20° C. or less. In some embodiments, the digestion incubation time is performed at a temperature of about any of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 42° C., 44° C., 46° C., 48° C., 50° C., 52° C., 54° C., 56° C., 58° C., or 60° C. In some embodiments, the reduction incubation time is performed at a room temperature.

In some embodiments, the proteolytic digestion technique comprises a digestion incubation time of about 12 hours to about 24 hours, such as about any of 13 hours, 14 hours, 15 hours, 16 hours, 17 hours, 18 hours, 19 hours, 20 hours, 21 hours, 22 hours, 23 hours, wherein the digestion incubation time is performed at a temperature of about 20° C. to about 40° C., such about any of 22° C., 24° C., 26° C., 28° C., 30° C., 31° C., 32° C., 33° C., 34° C., 35° C., 36° C., 37° C., 38° C., 39° C. In some embodiments, the proteolytic digestion technique comprises use of an amount a protease for each of one or more proteases, e.g., trypsin and/or LysC, of about 1:15 to about 1:45, such as about any of 1:20, 1:25, 1:30, 1:35, or 1:40 (as measured based on the amount of the protease to the amount of polypeptide in a sample or a derivative thereof), and a digestion incubation time of about 12 hours to about 24 hours, such as about any of 13 hours, 14 hours, 15 hours, 16 hours, 17 hours, 18 hours, 19 hours, 20 hours, 21 hours, 22 hours, 23 hours, wherein the digestion incubation time is performed at a temperature of about 20° C. to about 40° C., such about any of 22° C., 24° C., 26° C., 28° C., 30° C., 31° C., 32° C., 33° C., 34° C., 35° C., 36° C., 37° C., 38° C., 39° C. In some embodiments, the proteolytic digestion technique is performed on a plasma sample or a derivate thereof, wherein the proteolytic digestion technique comprises a 1:40 ratio of trypsin to polypeptide in the sample of the derivative thereof and a 1:40 ratio of LysC to polypeptide in the sample or the derivative thereof. In some embodiments, the proteolytic digestion technique is performed on a serum sample or a derivate thereof, wherein the proteolytic digestion technique comprises a 1:20 ratio of trypsin.

The digestion incubation temperature can be controlled by numerous techniques and combinations thereof. In some embodiments, the digestion incubation temperature is controlled by an ambient temperature, such as room temperature. In some embodiments, the digestion incubation temperature is controlled by a water bath. In some embodiments, the digestion incubation temperature is controlled by a heat block. In some embodiments, the digestion incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the digestion incubation time temperature are controlled by a technique described herein, such as cooling at room temperature or a ramp rate.

In some embodiments, the proteolytic digestion technique comprises subjecting a sample, or a derivative thereof, e.g., an alkylated sample (including an alkylated sample quenched with a neutralizing agent), to a thermal cycle. In some embodiments, the thermal cycle comprises subjecting the sample, or a derivative thereof, to one or more of: (a) a block starting temperature (b) block set temperature (the temperature for the digestion incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 20° C. to about 50° C., including about any of 22° C., 24° C., 26° C., 28° C., 30° C., 31° C., 32° C., 33° C., 34° C., 35° C., 36° C., 37° C., 38° C., 39° C., 40° C., 42° C., 44° C., 46° C., 48° C.

In some embodiments, the thermal cycle comprises: (a) a set block temperature of about 20° C. to about 50° C., including about any of 22° C., 24° C., 26° C., 28° C., 30° C., 31° C., 32° C., 33° C., 34° C., 35° C., 36° C., 37° C., 38° C., 39° C., 40° C., 42° C., 44° C., 46° C., 48° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 35° C., such as any of about 20° C. to about 35° C., or about 20° C. to about 25° C., (b) a set block temperature of about 20° C. to about 50° C., including about any of 22° C., 24° C., 26° C., 28° C., 30° C., 31° C., 32° C., 33° C., 34° C., 35° C., 36° C., 37° C., 38° C., 39° C., 40° C., 42° C., 44° C., 46° C., 48° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

In some embodiments, the proteolytic digestion technique further comprises quenching the one or more proteolytic enzymes. In some embodiments, quenching the one or more proteolytic enzymes comprises denaturing the one or more proteolytic enzymes. In some embodiments, quenching the one or more proteolytic enzymes comprise adding an amount of an acid. In some embodiments, the acid is formic acid (FA) or trifluoroacetic acid (TFA), or a mixture thereof. In some embodiments, the amount (as assessed based on the final concentration in the sample containing solution) of the acid added is about any of 0.1% v/v, 0.2% v/v, 0.3% v/v, 0.4% v/v, 0.5% v/v, 0.6% v/v, 0.7% v/v, 0.8% v/v, 0.9% v/v, 1% v/v, 1.1% v/v, 1.2% v/v, 1.3% v/v, 1.4% v/v, 1.5% v/v, 1.6% v/v, 1.7% v/v, 1.8% v/v, 1.9% v/v, or 2% v/v.

### V. Additional Techniques

In certain aspects, the method provided herein comprise subjecting the proteolytically digested sample comprising a proteolytic glycopeptide to one or more additional steps prior to subjecting the proteolytically digested sample, or a derivative thereof, to a liquid chromatography-mass spectrometry (LC-MS) technique using a liquid chromatography system and a mass spectrometer. In some embodiments, the LC system is online with the MS (i.e., eluate from the LC system is directly introduced to the MS). In some embodiments, the one or more additional steps do not include a desalting step performed outside of the LC system (such as an offline desalting technique).

In some embodiments, the method further comprises adding a standard to the proteolytically digested sample prior to the LC-MS technique. In some embodiments, the standard is a stable isotope-internal standard (SI-IS) peptide mixture.

In some embodiments, the biological sample is not subjected to a high-abundant protein depletion technique prior to the thermal denaturation technique. For example, in some embodiments, the high-abundant protein depletion technique removes highly abundant proteins present in a blood sample, such as serum albumin.

### E2. Methods for Performing a LC-MS Analysis of a Proteolytic Glycopeptide

In certain aspects, provided herein is a method for performing a LC-MS analysis on a sample comprising a proteolytic glycopeptide. In some embodiments, the liquid chromatography (LC) system is online with a mass spectrometer (i.e., proteolytic peptide species, including glycopeptides, are eluted from the LC system directing into the mass spectrometer via a mass spectrometer interface. In some embodiments, the LC technique comprises performing a chromatographic separation of one or more proteolytic peptides, including glycopeptides. In some embodiments, the one or more proteolytic peptides subjected to a chromatographic separation are obtained from a proteolytically digested sample, such as described herein. In some embodiments, the chromatographic separation is performed on a proteolytically digested sample, such as described herein, (e.g., no additional separation technique, such as a sample clean-up step, is performed to remove one or more components from proteolytically digested sample). In some embodiments, the chromatographic separation is performed on a proteolytically digested sample comprising at least about 5 mM of a buffer, such as ammonium bicarbonate. In some embodiments, the chromatographic separation is performed on a proteolytically digested sample comprising an amount, such as at least about 1 mM, of a reducing agent or a byproduct thereof, such as a stable six-membered ring with an internal disulfide bond derived from DTT. In some embodiments, the chromatographic separation is performed on a proteolytically digested sample comprising an amount, such as at least about 1 mM, of an alkylating agent or a byproduct thereof, such as iodide (I⁻) derived from IAA.

In some embodiments, the method comprises introducing the proteolytically digested sample to a LC-MS system. In some embodiments, the method comprises performing a chromatographic separation of the proteolytically digested sample.

In some embodiments, the chromatographic separation comprises a gradient separation performing using mixtures of an aqueous mobile phase and an organic mobile phase. In some embodiments, the chromatographic separation comprises isocratic period, such as a period of at least about 90%, such as at least about any of 91%, 92%, 93%, 94%, 95%, 96%, 97%, 98%, 99%, or 100%, of an aqueous mobile phase to produce the initial eluate that is diverted from the mass spectrometer interface.

In some embodiments, the LC system comprises a reversed-phase chromatography column. In some embodiments, the reversed-phase column comprises an alkyl moiety, such as C18.

The present application contemplates a diverse array of additional features of LC-MS techniques for analyzing a sample comprising a glycopeptide using a mass spectrometer. In some embodiments, the liquid chromatography system comprises a high performance liquid chromatography system. In some embodiments, the liquid chromatography system comprises an ultra-high performance liquid chromatography system. In some embodiments, the liquid chromatography system comprises a high-flow liquid chromatography system. In some embodiments, the liquid chromatography system comprises a low-flow liquid chromatography system, such as a micro-flow liquid chromatography system or a nano-flow liquid chromatography system. In some embodiments, the liquid chromatography system is coupled, such as directly interfaced, with a mass spectrometer.

In some embodiment, the mass spectrometry technique comprises an ionization technique. Ionization techniques contemplated by the present application include techniques capable of charging polypeptides and peptide products, including glycopeptides. Thus, in some embodiments, the ionization technique is electrospray ionization. In some embodiments, the ionization technique is nano-electrospray ionization. In some embodiments, the ionization technique is atmospheric pressure chemical ionization. In some embodiments, the ionization technique is atmospheric pressure photoionization.

A diverse array of mass spectrometers are contemplated as compatible with the description, include high-resolution mass spectrometers and low-resolution mass spectrometers. In some embodiments, the mass spectrometer is a time-of-flight (TOF) mass spectrometer. In some embodiments, the mass spectrometer is a quadrupole time-of-flight (Q-TOF) mass spectrometer. In some embodiments, the mass spectrometer is a quadrupole ion trap time-of-flight (QIT-TOF) mass spectrometer. In some embodiments, the mass spectrometer is an ion trap. In some embodiments, the mass spectrometer is a single quadrupole. In some embodiments, the mass spectrometer is a triple quadrupole (QQQ). In some embodiments, the mass spectrometer is an orbitrap. In some embodiments, the mass spectrometer is a quadrupole orbitrap. In some embodiments, the mass spectrometer is a fourier transform ion cyclotron resonance (FT) mass spectrometer. In some embodiments, the mass spectrometer is a quadrupole fourier transform ion cyclotron resonance (Q-FT) mass spectrometer. In some embodiments, the mass spectrometry technique comprises positive ion mode. In some embodiments, the mass spectrometry technique comprises negative ion mode. In some embodiments, the mass spectrometry technique comprises an ion mobility mass spectrometry technique.

In some embodiments, the LC-MS technique comprises processing obtained signals MS from the mass spectrometer. In some embodiments, the LC-MS technique comprises peak detection. In some embodiments, the LC-MS technique comprises determining ionization intensity of an ionized peptide product. In some embodiments, the LC-MS technique comprises determining peak height of an ionized peptide product. In some embodiments, the LC-MS technique comprises determining peak area of an ionized peptide product. In some embodiments, the LC-MS technique comprises determining peak volume of an ionized peptide product. In some embodiments, the LC-MS technique comprises identifying an ionized peptide product by amino acid sequence. In some embodiments, the LC-MS technique comprises determining the site of a post-translational modification of an ionized peptide, such as the site of a glycosylation. In some embodiments, the LC-MS technique comprises determining the glycan structure, or a characteristic thereof, of an ionized peptide product. In some embodiments, the LC-MS technique comprises manually validating the ionized peptide product acid sequence assignments. In some embodiments, the LC-MS technique comprises a quantification technique.

### F2. Exemplary Methods

In certain aspects, provided is a method for processing a proteolytically digested sample to produce a processed sample suitable for use in a liquid chromatography-mass spectrometry (LC-MS) analysis, wherein the proteolytically digested sample comprises a plurality of proteolytic polypeptides comprising at least one proteolytic glycopeptide, the method comprising subjecting the proteolytically digested sample to a solid phase extraction column comprising a reversed-phase medium to associate at least a portion of the plurality of proteolytic polypeptides with the reversed-phase medium, the subjecting comprising a polypeptide loading amount of about 50% or less of a binding capacity of the reversed-phase medium, wherein the binding capacity of the reversed-phase medium is based on an insulin load having 10% or less breakthrough; subjecting the reversed-phase medium comprising the associated proteolytic polypeptides to a wash buffer; and subjecting the reversed-phase medium comprising the associated proteolytic polypeptides to an elution buffer to produce the processed sample. In some embodiments, the reversed-phase medium comprises an alkyl-based moiety comprising an octadecyl carbon functional group (C18) covalently bound to a silica solid phase, wherein the silica solid phase comprises a plurality of particles having an average largest cross-sectional distance (such as a diameter, e.g., as measured by dynamic light scattering) of about 20 μm, and wherein the each particle of the plurality of particles comprises an average pore size of about 150 Å, and wherein the pores are derivitized with the alkyl-based moiety. In some embodiments, the solid phase extraction column comprising the reversed-phase medium has a column volume of about 5 μL. In some embodiments, the solid phase extraction column is an AssayMap 5 μL C18 cartridge (catalog no. 5190-6532; Agilent Technologies). In some embodiments, the reversed-phase medium comprises an underivitized polystyrene divinylbenzene hydrophobic reversed-phase resin, wherein the polystyrene divinylbenzene hydrophobic reversed-phase resin comprises a plurality of particles having an average largest cross-sectional distance (such as a diameter, e.g., as measured by dynamic light scattering) of about 20 μm, and wherein the each particle of the plurality of particles comprises an average pore size of about 100 Å. In some embodiments, the solid phase extraction column comprising the reversed-phase medium has a column volume of about 5 μL. In some embodiments, the solid phase extraction column is an AssayMAP 5 μL Reversed Phase (RP-S) cartridge (catalog no. G5496-60033; Agilent Technologies). In some embodiments, the proteolytic glycopeptide comprises a glycan structure comprising one or more sialic acid moieties.

In other aspects, provided is a method for processing a proteolytically digested sample to produce a processed sample suitable for use in a liquid chromatography-mass spectrometry (LC-MS) analysis, wherein the proteolytically digested sample comprises a plurality of proteolytic polypeptides comprising at least one proteolytic glycopeptide, the method comprising subjecting the proteolytically digested sample to a solid phase extraction column comprising a reversed-phase medium to associate at least a portion of the plurality of proteolytic polypeptides with the reversed-phase medium, the subjecting comprising a polypeptide loading concentration of about 0.6 μg/μL or less; subjecting the reversed-phase medium comprising the associated proteolytic polypeptides to a wash buffer; and subjecting the reversed-phase medium comprising the associated proteolytic polypeptides to an elution buffer to produce the processed sample. In some embodiments, the reversed-phase medium comprises an alkyl-based moiety comprising an octadecyl carbon functional group (C18) covalently bound to a silica solid phase, wherein the silica solid phase comprises a plurality of particles having an average largest cross-sectional distance (such as a diameter, e.g., as measured by dynamic light scattering) of about 20 μm, and wherein the each particle of the plurality of particles comprises an average pore size of about 150 Å, and wherein the pores are derivitized with the alkyl-based moiety. In some embodiments, the solid phase extraction column comprising the reversed-phase medium has a column volume of about 5 μL. In some embodiments, the solid phase extraction column is an AssayMap 5 μL C18 cartridge (catalog no. 5190-6532; Agilent Technologies). In some embodiments, the reversed-phase medium comprises an underivitized polystyrene divinylbenzene hydrophobic reversed-phase resin, wherein the polystyrene divinylbenzene hydrophobic reversed-phase resin comprises a plurality of particles having an average largest cross-sectional distance (such as a diameter, e.g., as measured by dynamic light scattering) of about 20 μm, and wherein the each particle of the plurality of particles comprises an average pore size of about 100 Å. In some embodiments, the solid phase extraction column comprising the reversed-phase medium has a column volume of about 5 μL. In some embodiments, the solid phase extraction column is an AssayMAP 5 μL Reversed Phase (RP-S) cartridge (catalog no. G5496-60033; Agilent Technologies). In some embodiments, the proteolytic glycopeptide comprises a glycan structure comprising one or more sialic acid moieties.

In other aspects, provided is a method for processing a proteolytically digested sample to produce a processed sample suitable for use in a liquid chromatography-mass spectrometry (LC-MS) analysis, wherein the proteolytically digested sample comprises a plurality of proteolytic polypeptides comprising at least one proteolytic glycopeptide, the method comprising subjecting the proteolytically digested sample to a solid phase extraction column comprising a reversed-phase medium to associate at least a portion of the plurality of proteolytic polypeptides with the reversed-phase medium; subjecting the reversed-phase medium comprising the associated proteolytic polypeptides to a wash buffer at a wash flow rate of about 0.1 column volumes/minute to about 2 column volumes/minute; and subjecting the reversed-phase medium comprising the associated proteolytic polypeptides to an elution buffer to produce the processed sample. In some embodiments, the reversed-phase medium comprises an alkyl-based moiety comprising an octadecyl carbon functional group (C18) covalently bound to a silica solid phase, wherein the silica solid phase comprises a plurality of particles having an average largest cross-sectional distance (such as a diameter, e.g., as measured by dynamic light scattering) of about 20 μm, and wherein the each particle of the plurality of particles comprises an average pore size of about 150 Å, and wherein the pores are derivitized with the alkyl-based moiety. In some embodiments, the solid phase extraction column comprising the reversed-phase medium has a column volume of about 5 μL. In some embodiments, the solid phase extraction column is an AssayMap 5 μL C18 cartridge (catalog no. 5190-6532; Agilent Technologies). In some embodiments, the reversed-phase medium comprises an underivitized polystyrene divinylbenzene hydrophobic reversed-phase resin, wherein the polystyrene divinylbenzene hydrophobic reversed-phase resin comprises a plurality of particles having an average largest cross-sectional distance (such as a diameter, e.g., as measured by dynamic light scattering) of about 20 μm, and wherein the each particle of the plurality of particles comprises an average pore size of about 100 Å. In some embodiments, the solid phase extraction column comprising the reversed-phase medium has a column volume of about 5 μL. In some embodiments, the solid phase extraction column is an AssayMAP 5 μL Reversed Phase (RP-S) cartridge (catalog no. G5496-60033; Agilent Technologies). In some embodiments, the proteolytic glycopeptide comprises a glycan structure comprising one or more sialic acid moieties.

In certain aspects, provided is a method for processing a proteolytically digested sample to produce a processed sample suitable for use in a liquid chromatography-mass spectrometry (LC-MS) analysis, wherein the proteolytically digested sample comprises a plurality of proteolytic polypeptides comprising at least one proteolytic glycopeptide, the method comprising subjecting the proteolytically digested sample to a solid phase extraction column comprising a reversed-phase medium to associate at least a portion of the plurality of proteolytic polypeptides with the reversed-phase medium, the subjecting comprising a polypeptide loading amount of about 50% or less of a binding capacity of the reversed-phase medium, wherein the binding capacity of the reversed-phase medium is based on an insulin load having 10% or less breakthrough, and wherein the subjecting comprising a polypeptide loading concentration of about 0.6 μg/μL or less; subjecting the reversed-phase medium comprising the associated proteolytic polypeptides to a wash buffer; and subjecting the reversed-phase medium comprising the associated proteolytic polypeptides to an elution buffer to produce the processed sample. In some embodiments, the reversed-phase medium comprises an alkyl-based moiety comprising an octadecyl carbon functional group (C18) covalently bound to a silica solid phase, wherein the silica solid phase comprises a plurality of particles having an average largest cross-sectional distance (such as a diameter, e.g., as measured by dynamic light scattering) of about 20 μm, and wherein the each particle of the plurality of particles comprises an average pore size of about 150 Å, and wherein the pores are derivitized with the alkyl-based moiety. In some embodiments, the solid phase extraction column comprising the reversed-phase medium has a column volume of about 5 μL. In some embodiments, the solid phase extraction column is an AssayMap 5 μL C18 cartridge (catalog no. 5190-6532; Agilent Technologies). In some embodiments, the reversed-phase medium comprises an underivitized polystyrene divinylbenzene hydrophobic reversed-phase resin, wherein the polystyrene divinylbenzene hydrophobic reversed-phase resin comprises a plurality of particles having an average largest cross-sectional distance (such as a diameter, e.g., as measured by dynamic light scattering) of about 20 μm, and wherein the each particle of the plurality of particles comprises an average pore size of about 100 Å. In some embodiments, the solid phase extraction column comprising the reversed-phase medium has a column volume of about 5 μL. In some embodiments, the solid phase extraction column is an AssayMAP 5 μL Reversed Phase (RP-S) cartridge (catalog no. G5496-60033; Agilent Technologies). In some embodiments, the proteolytic glycopeptide comprises a glycan structure comprising one or more sialic acid moieties.

In certain aspects, provided is a method for processing a proteolytically digested sample to produce a processed sample suitable for use in a liquid chromatography-mass spectrometry (LC-MS) analysis, wherein the proteolytically digested sample comprises a plurality of proteolytic polypeptides comprising at least one proteolytic glycopeptide, the method comprising subjecting the proteolytically digested sample to a solid phase extraction column comprising a reversed-phase medium to associate at least a portion of the plurality of proteolytic polypeptides with the reversed-phase medium, the subjecting comprising a polypeptide loading amount of about 50% or less of a binding capacity of the reversed-phase medium, wherein the binding capacity of the reversed-phase medium is based on an insulin load having 10% or less breakthrough; subjecting the reversed-phase medium comprising the associated proteolytic polypeptides to a wash buffer at a wash flow rate of about 0.1 column volumes/minute to about 2 column volumes/minute; and subjecting the reversed-phase medium comprising the associated proteolytic polypeptides to an elution buffer to produce the processed sample. In some embodiments, the reversed-phase medium comprises an alkyl-based moiety comprising an octadecyl carbon functional group (C18) covalently bound to a silica solid phase, wherein the silica solid phase comprises a plurality of particles having an average largest cross-sectional distance (such as a diameter, e.g., as measured by dynamic light scattering) of about 20 μm, and wherein the each particle of the plurality of particles comprises an average pore size of about 150 Å, and wherein the pores are derivitized with the alkyl-based moiety. In some embodiments, the solid phase extraction column comprising the reversed-phase medium has a column volume of about 5 μL. In some embodiments, the solid phase extraction column is an AssayMap 5 μL C18 cartridge (catalog no. 5190-6532; Agilent Technologies). In some embodiments, the reversed-phase medium comprises an underivitized polystyrene divinylbenzene hydrophobic reversed-phase resin, wherein the polystyrene divinylbenzene hydrophobic reversed-phase resin comprises a plurality of particles having an average largest cross-sectional distance (such as a diameter, e.g., as measured by dynamic light scattering) of about 20 μm, and wherein the each particle of the plurality of particles comprises an average pore size of about 100 Å. In some embodiments, the solid phase extraction column comprising the reversed-phase medium has a column volume of about 5 μL. In some embodiments, the solid phase extraction column is an AssayMAP 5 μL Reversed Phase (RP-S) cartridge (catalog no. G5496-60033; Agilent Technologies). In some embodiments, the proteolytic glycopeptide comprises a glycan structure comprising one or more sialic acid moieties.

In other aspects, provided is a method for processing a proteolytically digested sample to produce a processed sample suitable for use in a liquid chromatography-mass spectrometry (LC-MS) analysis, wherein the proteolytically digested sample comprises a plurality of proteolytic polypeptides comprising at least one proteolytic glycopeptide, the method comprising subjecting the proteolytically digested sample to a solid phase extraction column comprising a reversed-phase medium to associate at least a portion of the plurality of proteolytic polypeptides with the reversed-phase medium, the subjecting comprising a polypeptide loading concentration of about 0.6 μg/μL or less; subjecting the reversed-phase medium comprising the associated proteolytic polypeptides to a wash buffer at a wash flow rate of about 0.1 column volumes/minute to about 2 column volumes/minute; and subjecting the reversed-phase medium comprising the associated proteolytic polypeptides to an elution buffer to produce the processed sample. In some embodiments, the reversed-phase medium comprises an alkyl-based moiety comprising an octadecyl carbon functional group (C18) covalently bound to a silica solid phase, wherein the silica solid phase comprises a plurality of particles having an average largest cross-sectional distance (such as a diameter, e.g., as measured by dynamic light scattering) of about 20 μm, and wherein the each particle of the plurality of particles comprises an average pore size of about 150 Å, and wherein the pores are derivitized with the alkyl-based moiety. In some embodiments, the solid phase extraction column comprising the reversed-phase medium has a column volume of about 5 μL. In some embodiments, the solid phase extraction column is an AssayMap 5 μL C18 cartridge (catalog no. 5190-6532; Agilent Technologies). In some embodiments, the reversed-phase medium comprises an underivitized polystyrene divinylbenzene hydrophobic reversed-phase resin, wherein the polystyrene divinylbenzene hydrophobic reversed-phase resin comprises a plurality of particles having an average largest cross-sectional distance (such as a diameter, e.g., as measured by dynamic light scattering) of about 20 μm, and wherein the each particle of the plurality of particles comprises an average pore size of about 100 Å. In some embodiments, the solid phase extraction column comprising the reversed-phase medium has a column volume of about 5 μL. In some embodiments, the solid phase extraction column is an AssayMAP 5 μL Reversed Phase (RP-S) cartridge (catalog no. G5496-60033; Agilent Technologies). In some embodiments, the proteolytic glycopeptide comprises a glycan structure comprising one or more sialic acid moieties.

In other aspects, provided is a method for processing a proteolytically digested sample to produce a processed sample suitable for use in a liquid chromatography-mass spectrometry (LC-MS) analysis, wherein the proteolytically digested sample comprises a plurality of proteolytic polypeptides comprising at least one proteolytic glycopeptide, the method comprising: subjecting the proteolytically digested sample to a solid phase extraction column comprising a reversed-phase medium to associate at least a portion of the plurality of proteolytic polypeptides with the reversed-phase medium, the subjecting comprising a polypeptide loading amount of about 50% or less of a binding capacity of the reversed-phase medium, wherein the binding capacity of the reversed-phase medium is based on an insulin load having 10% or less breakthrough, and a polypeptide loading concentration of about 0.6 μg/μL or less; subjecting the reversed-phase medium comprising the associated proteolytic polypeptides to a wash buffer at a wash flow rate of about 0.1 column volumes/minute to about 2 column volumes/minute; and subjecting the reversed-phase medium comprising the associated proteolytic polypeptides to an elution buffer to produce the processed sample. In some embodiments, the reversed-phase medium comprises an alkyl-based moiety comprising an octadecyl carbon functional group (C18) covalently bound to a silica solid phase, wherein the silica solid phase comprises a plurality of particles having an average largest cross-sectional distance (such as a diameter, e.g., as measured by dynamic light scattering) of about 20 μm, and wherein the each particle of the plurality of particles comprises an average pore size of about 150 Å, and wherein the pores are derivitized with the alkyl-based moiety. In some embodiments, the solid phase extraction column comprising the reversed-phase medium has a column volume of about 5 μL. In some embodiments, the solid phase extraction column is an AssayMap 5 μL C18 cartridge (catalog no. 5190-6532; Agilent Technologies). In some embodiments, the reversed-phase medium comprises an underivitized polystyrene divinylbenzene hydrophobic reversed-phase resin, wherein the polystyrene divinylbenzene hydrophobic reversed-phase resin comprises a plurality of particles having an average largest cross-sectional distance (such as a diameter, e.g., as measured by dynamic light scattering) of about 20 μm, and wherein the each particle of the plurality of particles comprises an average pore size of about 100 Å. In some embodiments, the solid phase extraction column comprising the reversed-phase medium has a column volume of about 5 μL. In some embodiments, the solid phase extraction column is an AssayMAP 5 μL Reversed Phase (RP-S) cartridge (catalog no. G5496-60033; Agilent Technologies). In some embodiments, the proteolytic glycopeptide comprises a glycan structure comprising one or more sialic acid moieties.

### G2. Samples and Components Thereof

The methods provided herein are contemplated to be suitable for analyzing a diverse array of samples, such as biological samples. In some embodiments, the sample is a blood sample, such as a whole blood sample. In some embodiments, the sample is a plasma sample. In some embodiments, the sample is a serum sample. In some embodiments, the sample is a tissue sample. Plasma is a fluid component of blood that is obtained when a clotting-prevention agent is added to whole blood and then the tube is centrifuged to separate the cellular material. The upper lighter colored liquid layer in the tube is removed as plasma. Common anti-coagulant agents are EDTA (ethylenediaminetetraacetic acid), heparin, and citrate. Serum is a fluid obtained when whole blood is allowed to clot in a tube and then centrifuged so that the clotted blood, including red cells, are at the bottom of the collection tube, leaving a straw-colored liquid above the clot. The straw-colored liquid in the tube is removed as serum.

The methods provided herein are particularly useful for the analysis of biological samples comprising a glycoprotein, such as to generate glycopeptide containing specimens for analysis with a mass spectrometer. The methods provided herein, in some embodiments, enable the analysis of glycopeptides that elute during early or late phases of a reversed-phase chromatographic separation and are typically missed during conventional mass spectrometry approaches. For the situation where a sample contains hydrophilic salts and hydrophilic glycopeptides, it can be challenging to desalt the sample with a C18 solid phase extraction material without removing a significant portion of the hydrophilic glycopeptides that are needed for an analysis of the sample. For example, glycopeptides with an overall hydrophilic character may elute from a reversed-phase material, such as in a desalting column, and are washed away or not introduced to the mass spectrometer during a data acquisition phase of a mass spectrometry technique. In some embodiments, glycopeptides with an overall hydrophobic character may have a high affinity for a reversed-phase material, such as in a desalting column or a chromatography column, and are not properly eluted from a desalting column or during a data acquisition portion of a mass spectrometry technique.

In some embodiments, the method comprises an upstream sample preparation technique, such as for obtaining plasma or serum from a blood sample, performed prior to methods for proteolytically digesting a sample. In some embodiments, the upstream sample preparation technique comprises a cell lysis step. In some embodiments, the upstream sample preparation technique comprises a filtration step. In some embodiments, the upstream sample preparation technique comprises a dilution step. In some embodiments, the upstream sample preparation technique comprises a protein concentration determination step.

In some embodiments, the sample is obtained from an individual. In some embodiments, the sample is obtained from a human individual.

### H2. Systems, Kits, and Compositions

In certain aspects, contemplated herein are systems, kits, and compositions useful for performing the methods described herein. In some embodiments, provided herein is a system, kit, and/or composition useful for performing a proteolytic digestion of a biological sample comprising a glycoprotein as described herein. In some embodiments, provided herein is a system, kit, and/or composition useful for performing a LC-MS analysis of a proteolytic glycopeptide as described herein. In some embodiments, provided herein is a system, kit, and/or composition useful for performing a proteolytic digestion of a biological sample comprising a glycoprotein followed by LC-MS analysis of the proteolytic glycopeptide produced therefrom.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### Section 3—Absorbent or Bibulous Members Having a Polypeptide Standard and Configured for Deposition of a Blood Sample and LC-MS Analysis of Glycopeptides Therefrom

Provided herein, in certain aspects, is an absorbent or bibulous member, such as a blood spot card, comprising one or more extraction internal standards comprising at least one polypeptide standard deposited thereon prior to deposition of a blood sample. In other aspects, provided herein are one or more extraction internal standards comprising a polypeptide standard, including mixture of polypeptide standards, suitable for use in glycopolypeptide LC-MS analyses of samples obtained from an absorbent or bibulous member, such as a dried blood spot card. In other aspects, provided herein are methods for performing a liquid chromatography-mass spectrometry (LC-MS) analysis of a proteolytic glycopeptide derived from a blood sample deposited on an absorbent or bibulous member, such as a dried blood spot card, such as for the analysis of one or more biomarkers, e.g., biomarkers for the diagnosis of ovarian cancer. In other aspects, provided herein are one or more biomarkers for assessing ovarian cancer in an individual suitable for assessment of polypeptide content obtained from an absorbent or bibulous member, such as a blood spot card. The disclosure of the present application is based on the inventors' unique perspective and unexpected findings regarding the use of absorbent or bibulous members, such as blood spot cards, for the analysis of glycopolypeptides, such as proteolytic glycopeptides. As described herein, the inventors identified that the use of one or more extraction internal standards applied to an absorbent or bibulous member, such as a blood spot card, prior to deposition of a blood sample can facilitate analytical aspects of an LC-MS analysis of proteolytic peptides therefrom, such as one or more proteolytic glycopeptide. As described herein, the one or more extraction internal standards enable determination of any one or more of extraction efficiency, digestion efficiency, and a sample migration pattern such as to improve the analysis of polypeptide content, such as one or more proteolytic glycopeptides, extracted from an absorbent or bibulous member. Specifically, as described herein, it was unexpectedly found that the taught methods involving absorbent or bibulous members, such as blood spot cards, and one or more extraction internal standards enable performance of an ovarian cancer biomarker screen to accurately and reliably determine the presence of benign or non-benign ovarian cancer. Such results obtained using biomarkers useful for the techniques involving absorbent or bibulous member samples described herein were in agreement with a biomarkers for an established liquid blood-based assay. Furthermore, using the methods of performing a LC-MS analysis involving the absorbent or bibulous members described herein, the inventors identified one or more biomarkers useful for assessing ovarian cancer in an individual. In addition to the significant advantages provided by the subject matter described herein that enable LC-MS analysis of polypeptide content, such as one or more proteolytic glycopeptides, the subject matter provided herein benefits from the use of dried blood sample, e.g., reduced risk of live blood biohazards, inhibition of enzymatic activity (e.g., proteases and glycotransferases), ease of obtaining a blood sample from an individual (including the small amount of blood sample required), and ease of transportation of a blood sample from the site of obtaining to a site of analysis.

Thus, in some aspects, provided herein is a method for performing a liquid chromatography-mass spectrometry (LC-MS) analysis of a proteolytic glycopeptide derived from a blood sample deposited on a delimited zone of an absorbent or bibulous member, wherein the blood sample comprises a plurality of polypeptides comprising at least one glycoprotein, the method comprising: extracting at least a portion of the plurality of polypeptides and one or more extraction internal standards from the absorbent or bibulous member to obtain an extracted sample, wherein the absorbent or bibulous member comprises the one or more extraction internal standards prior to deposition of the blood sample within the delimited zone, and wherein at least one of the one or more extraction internal standards comprises a polypeptide standard; subjecting the extracted sample or a derivative thereof to a proteolytic digestion technique to produce a proteolytically digested sample comprising the proteolytic glycopeptide; introducing at least a portion of the proteolytically digested sample to a liquid chromatography (LC) system of a LC-MS system; and performing the LC-MS analysis on at least the proteolytic glycopeptide and the one or more extraction internal standards.

In other aspects, provided herein is a method for performing a liquid chromatography-mass spectrometry (LC-MS) analysis of a proteolytic glycopeptide derived from a blood sample from an individual deposited on a delimited zone of an absorbent or bibulous member, the method comprising: obtaining an absorbent or bibulous member comprising a blood sample from the individual deposited thereon, wherein the absorbent or bibulous member comprises one or more extraction internal standards deposited and dried prior to deposition of the blood sample on the absorbent or bibulous member, and wherein the absorbent or bibulous member comprising the blood sample contains at least a portion of the blood sample and the one or more extraction internal standards in an overlapping area of the absorbent or bibulous member; extracting at least a portion of the plurality of polypeptides and the one or more extraction internal standards from the absorbent or bibulous member to obtain an extracted sample; subjecting the extracted sample or a derivative thereof to a proteolytic digestion technique to produce a proteolytically digested sample comprising the proteolytic glycopeptide; introducing at least a portion of the proteolytically digested sample to a liquid chromatography (LC) system of a LC-MS system; and performing an LC-MS analysis to quantify one or more biomarkers of ovarian cancer and the one or more extraction internal standards, and wherein at least one of the one or more biomarkers is a glycopeptide.

In other aspects, provided herein is an absorbent or bibulous member, such as a blood spot card, comprising one or more extraction internal standard, wherein the one or more extraction internal standards comprises at least one polypeptide standard, and wherein the blood spot card does not comprise a blood sample deposited thereon. In some embodiments, the one or more extraction standards are deposited on the absorbent or bibulous member, such as a blood spot card, in a delimited zone, such as where a blood sample is deposited.

### B3. Example Mass Spectrometry and Sample Preparation Workflow

For purposes of orientation and illustration of the description herein, provided in this section are example aspects of sample preparation and mass spectrometry workflows (FIGS. 1A-1C) for analyzing the composition of a peptide and/or glycopeptide using a mass spectrometer. Subsequent sections are provided with more details regarding certain inventive features related to methods for proteolytically digesting a biological sample, such as a blood sample, comprising a glycoprotein, methods of performing a LC-MS analysis of a proteolytic glycopeptide, and mass spectrometry workflows involving any combination of elements thereof.

FIG. 1A is a schematic of an example workflow 100 for a peptide structure analysis, including of glycopeptides. The workflow 100 may include various operations including, for example, sample collection 102 using an absorbent or bibulous member, such as a blood spot card, sample intake 104, sample preparation and mass spectrometry processing 106, and data analysis 108.

Sample collection 102 may include, for example, obtaining a biological sample, such as a blood sample, 112 from an individual 114. Relevant to the methodology described herein, a biological sample 112 may take the form of a blood sample obtained via one or more sampling methods, such as a finger prick produced using a lancet, and deposited on an absorbent or bibulous member, such as a blood spot card. In some embodiments, many aliquots of the biological sample 112 are obtained 118, such as via deposition of a blood sample from an individual onto a plurality of absorbent or bibulous members or a plurality of blood deposition sites on an absorbent or bibulous member, such as a blood spot card. In some embodiments, the absorbent or bibulous members performs a separation of the blood sample from the individual, such as to produce a serum sample, a plasma sample, and/or extraction of a blood cell (e.g., white blood cell (WBC), red blood cell (RBC) sample. In some embodiments, the biological sample, such as the blood sample, 112 may include nucleotides (e.g., ssDNA, dsDNA, RNA), organelles, amino acids, peptides, proteins, carbohydrates, glycoproteins, or any combination thereof.

Sample intake 104 may include one or more various operations such as, for example, aliquoting, labeling, registering, processing, storing, thawing, and/or other types of operations involved with preparing a sample for sample preparation and mass spectrometry processing. In some embodiments, sample intake 104 comprises separating one or portions of an absorbent or bibulous member, such as punching one or more chads out of a blood spot card with a hole punching device.

Sample preparation and mass spectrometry processing 106 may include, for example, one or more operations to form set of peptide structures 122, such as a proteolytic peptide and/or a proteolytic glycopeptide. In some embodiments, the sample preparation comprises extracting one or more polypeptides from an absorbent or bibulous member, such as a blood spot card, or a portion thereof with an extraction buffer. In some embodiments, the extracted polypeptide content is further processed, such as via subjecting to a precipitation technique. In some embodiments, the sample preparation includes subjecting a blood sample, or a portion thereof, to a proteolytic digestion. Mass spectrometry processing 124 may include, for example, liquid chromatography, introducing species from the sample, and/or derived therefrom, to a mass spectrometer, and data acquisition, such as using a multiple reaction monitoring (MRM) technique. MRM is a mass spectrometry method in which a precursor ion of a particular m/z value, including window thereof, (e.g., peptide analyte) is selected in the first quadrupole (Q1) and transmitted to the second quadrupole (Q2) for fragmentation. The resulting product ions are then transmitted to the third quadrupole (Q3), which detects only product ions with selected predefined m/z values. In some embodiments, the predefined m/z value, including window thereof, selected in the first quadrupole and a predefined m/z value, including window thereof, may be expressed as a MRM transition. Dynamic MRM (dMRM) is a variant of MRM. In dynamic MRM mode, MRM transition lists are scheduled throughout an LC/MS run based on the retention time window for each analyte. In this way, analytes are only monitored while they are eluting from the LC and therefore the MS scan time is not wasted by monitoring the analytes when they are not expected.

Data analysis 108 may include, for example, peptide structure analysis 126, e.g., determining the amino acid sequence of a peptide, determining a site of a post-translational modification, and/or determining a glycan composition and/or structure. In some embodiments, data analysis 108 also includes output generation 110. In some embodiments, output generation 110 may be considered a separate operation from data analysis 108. Output generation 110 may include, for example, generating final output 128 based on the results of peptide structure analysis 126. In some embodiments, the final output 128 may be used for one or more downstream purposes, such as research, diagnosis, and/or treatment, and may be sent to a remote system 130.

In certain aspects, the workflow 100 may optionally exclude one or more of the operations described herein and/or may optionally include one or more other steps or operations other than those described herein (e.g., in addition to and/or instead of those described herein).

FIG. 1B is a schematic of an example workflow 200 for certain sample preparation techniques 106, some of which may be optionally used in methods provided herein. In some embodiments, the workflow 200 comprises a denaturation step 202, such as to unfold and/or linearize a polypeptide to expose one or more cleavage sites. In some embodiments, the workflow 200 comprises a reduction step 202, such as to cleave disulfide bonds. In some embodiments, the workflow 200 comprises an alkylation technique 204, such as to modify cysteine residues to prevent reformation of a disulfide bond. In some embodiments, the workflow 200 comprises a protease digestion technique 206, such as to produce proteolytic peptides, including proteolytic glycopeptides. Box 205 can represent the R group of an amino acid such as, for example, an R group of arginine or lysine that typically will direct a tryptic cleavage. In some embodiments, the workflow 200 may comprise a post-digestion procedure 207, such as any of a desalting technique, addition of a standard, aliquoting, and/or preparation for a mass spectrometry analysis.

FIG. 1C is a schematic of an example workflow for certain mass spectrometry processing techniques 106, some of which may be optionally used in methods provided herein. In some embodiments, the workflow comprises a quantification technique 208 using a mass spectrometer, such as a liquid chromatography-mass spectrometry system. In some embodiments, the workflow comprises a quality control technique 210 configured to optimize data quality. In some embodiments, measures can be put in place allowing only errors within acceptable ranges outside of an expected value. In some embodiments, employing statistical models (e.g., using Westgard rules) can assist in quality control 210. For example, quality control 210 may include, for example, assessing the retention time and abundance of representative peptide structures (e.g., glycosylated and/or aglycosylated) and spiked-in internal standards, in either every sample, or in each quality control sample (e.g., pooled serum digest). In some embodiments, the workflow comprises a peak integration and normalization technique 212 to process the data that has been generated and transform the data into a format for analysis. For example, peak integration and normalization 212 may include converting abundance data for various product ions that were detected for a selected peptide structure into a single quantification metric (e.g., a relative quantity, an adjusted quantity, a normalized quantity, a relative concentration, an adjusted concentration, a normalized concentration, etc.) for that peptide structure. In some embodiments, peak integration and normalization 212 may be performed using one or more of the techniques described in U.S. Patent Publication No. 2020/0372973 A1 and/or US Patent Publication No. 2020/0240996A1, the disclosures of which are incorporated by reference herein in their entireties

### C3. Absorbent or Bibulous Members and Methods of Use Thereof

Provided herein, in certain aspects, are absorbent or bibulous members, such as a blood spot card or a lateral flow blood sample device, comprising one or more extraction internal standards, wherein at least one of the one or more extraction internal standards is a polypeptide standard. The absorbent or bibulous member and extraction internal standards, including methods associated therewith, are described in more detail below. The modular discussion of these features of the application is not intended to limit the scope of the description provided herein, and one of ordinary skill in the art will readily appreciate that various features can be combined.

### I. Absorbent or Bibulous Members

The absorbent or bibulous members encompassed herein may take many forms and, in some embodiments, perform one or more blood sample processing functions. Generally speaking, the absorbent or bibulous members described herein serve as a platform to accept a blood sample, or a derivative thereof, from an individual, wherein the blood sample, or the derivative thereof, can dry on or within the absorbent or bibulous member. Subsequently, the absorbent or bibulous member comprising a blood sample can be processed, e.g., shipped to a laboratory or subjected to an extraction technique. The absorbent or bibulous members described herein enhance the stability of analytes at ambient or elevated environmental temperatures (such as by inhibiting enzymatic activity in whole blood) and simplify the transportation of sample by avoiding the need for a cold chain. Blood spot cards are well known in the art, e.g., see U.S. Pat. Nos. 3,838,012; 9,198,609; and 10,422,729, which are hereby incorporated herein by reference in their entirety.

In some embodiments, the absorbent or bibulous member is a blood spot card. In some embodiments, the blood spot card comprises a porous material or a mesh material on which a blood sample, or portion thereof, is deposited and/or received. In some embodiments, the blood spot card comprises a filter paper material, such as an absorbent filter paper. In some embodiments, the filter paper material comprises a cellulose-based paper. In some embodiments, the filter paper material does not bind polypeptide content in a manner such that said polypeptide content cannot be later extracted from the filter paper material. In some embodiments the filter paper material prevents or reduces sample hemolysis. In some embodiments, the filter paper material is Watman paper. In some embodiments, the blood spot card is a Whatman 903 Proteinsaver card.

In some embodiments, the absorbent or bibulous member is a blood spot card comprising one or more delimited zones configured to guide placement of a blood sample. Such delimited zones may be any shape or size, and have any placement on the blood spot card. In some embodiments, the delimited zone has a surface area of about 100 mm² to about 1,000 mm², such as about any of 150 mm², 200 mm², 250 mm², 300 mm², 350 mm², 400 mm², 450 mm², 500 mm², 600 mm², 700 mm², 800 mm², or 900 mm². In some embodiments, the delimited zone has a surface area of about 1,000 mm² or less, such as about any of 900 mm² or less, 800 mm² or less, 700 mm² or less, 600 mm² or less, 500 mm² or less, 450 mm² or less, 400 mm² or less, 350 mm² or less, 300 mm² or less, 250 mm² or less, 200 mm² or less, 150 mm² or less, or 100 mm² or less. In some embodiments, the delimited zone forms a circle. In some embodiments, the delimited zone is marked, such as visible to the naked eye, but a line or a portion thereof, such as a dashed line. In some embodiments, the blood spot card comprises one or more additional markings, such as identifying information.

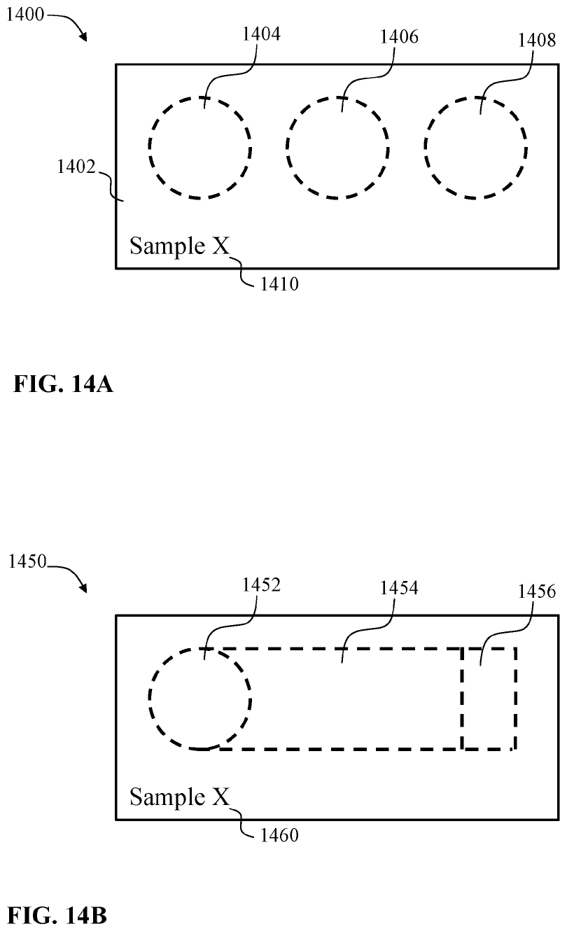

FIG. 14A provides an example blood spot card 1400 having three delimited zones 1404, 1406, 1408. The blood spot card 1400 comprises a filter paper material 1402. The delimited zones 1404, 1406, 1408 are marked by dashed line circles and direct the placement of a blood sample. The blood spot card 1400 further comprises a marking 1410 for the identification of the sample.

In some embodiments, the blood spot card is a patterned blood spot card comprising a feature to control the area of the card exposed to a blood sample. In some embodiments, the patterned blood spot card comprises a hydrophobic material, such as a wax, to control the area of the card exposed to a blood sample.

In some embodiments, the blood spot card further comprises one or more extraction internal standards, wherein at least one of the extraction internal standards comprises a polypeptide standard. The extraction internal standards may be placed in a variety of configurations on the blood spot card such that once the blood sample is applied and allowed to dry, a portion of the blood spot card can be separated, such as by punching a chad from the blood spot card, the portion of the blood spot card comprising at least a portion of the blood sample and the one or more extraction internal standards. In some embodiments, the one or more extraction internal standards are deposited in a delimited zone of the blood spot card such that the one or more extraction internal standards are dried on the blood spot card prior to deposition of a blood sample. In some embodiments, the one or more extraction internal standards are deposited in a substantially uniform manner within the delimited zone. In some embodiments, the one or more extraction internal standards are deposited in a patterned manner within the delimited zone. In some embodiments, the delimited zone of a blood spot card comprises a known concentration of one or more extraction internal standards. In some embodiments, the method described herein comprise determining the total amount of a blood sample component and/or one or more extraction internal standards deposited on an absorbent or bibulous member, such as a blood spot card, based on the size of the one or more chads used to obtain polypeptide content for LC-MS analysis (e.g., a correction factor based on the size of the one or more chads and the size of a delimited area comprising a blood sample (or a portion thereof) and one or more extraction internal standards.

In some embodiments, the absorbent or bibulous member comprises a lateral flow element useful for performing a blood sample processing step. In some embodiments, the absorbent or bibulous member comprising a lateral flow element comprises (a) a delimited zone for deposition of a blood sample, and (b) a lateral flow element. In some embodiments, the absorbent or bibulous member comprises a zone, such as a delimited zone, wherein the component of the blood sample, such as a plasma or serum sample is obtained and allowed to dry thereon. In some embodiments, the zone comprising the plasma or serum sample is a portion of the lateral flow element.

FIG. 14B provides an example absorbent or bibulous member comprising a lateral flow element 1450. The absorbent or bibulous member 1400 comprises a delimited zone 1452 for deposition of a blood sample operably connected with a lateral flow zone 1454 configured for separation of one or more components of the blood sample. The absorbent or bibulous member 1450 comprises a zone 1456 wherein the desired separated blood component can be obtained therefrom, e.g., a zone where a plasma or serum sample is dried thereon. The zone 1456 can be distal from and/or spaced apart from the delimited zone 1452. The absorbent or bibulous member 1450 further comprises a marking 1460 for the identification of the sample.

In some embodiments, the absorbent or bibulous member comprising a lateral flow element further comprises one or more extraction internal standards, wherein at least one of the extraction internal standards comprises a polypeptide standard. The extraction internal standards may be placed in a variety of configurations on the absorbent or bibulous member comprising a lateral flow element such that once the blood sample is applied and allowed to be processed and dry, a portion of the absorbent or bibulous member can be separated, such as by punching a chad from the absorbent or bibulous member, the portion of the absorbent or bibulous member comprising at least a portion of the blood sample and the one or more extraction internal standards. In some embodiments, the one or more extraction internal standards are deposited on a delimited zone configured for deposition of a blood sample. In some embodiments, the one or more extraction internal standards are deposited on a lateral flow element configured for processing a blood sample. In some embodiments, the one or more extraction internal standards are deposited on a zone configured for receipt of a component of a blood sample, such as a plasma or serum sample. Various combinations of the placement of the one or more extraction internal standards are encompassed by the description provided herein. In some embodiments, the absorbent or bibulous member comprising a lateral flow element comprises a plurality of populations of the one or more extraction internal standards, wherein the populations may of the extraction internal standards may be distinguished from one another (e.g., contain different polypeptide standards). In some embodiments, at least one population provides distinct information as compared to another population. For example, in some embodiments, the absorbent or bibulous member comprising a lateral flow element comprises a first population of the one or more extraction internal standards deposited on a delimited zone configured for deposition of a blood sample, wherein such population is useful for assessing sample migration to the zone for receipt of the processed sample. In some embodiments, the absorbent or bibulous member comprises a lateral flow element configured to separate whole blood into a portion of plasma, wherein the whole blood is deposited at the delimited zone and then a liquid portion of the whole blood laterally flows from the delimited zone to a distal zone, wherein the distal zone contains the portion of the plasma.

In some embodiments, the absorbent or bibulous member, including a single delimited zone of the absorbent or bibulous member, is configured to accept a blood sample having a volume of about 250 μL or less, such as about any of 225 μL or less, 200 μL or less, 175 μL or less, 150 μL or less, 125 μL or less, 100 μL or less, 75 μL or less, 50 μL or less, or 25 μL or less. In some embodiments, the absorbent or bibulous member, including a single delimited zone of the absorbent or bibulous member, is configured to accept a blood sample having a volume of about any of 250 μL, 225 μL, 200 μL, 175 μL, 150 μL, 125 μL, 100 μL, 75 μL, 50 μL, or 25 μL. In some embodiments, the absorbent or bibulous member, including a single delimited zone of the absorbent or bibulous member, is configured to accept a blood sample obtained from lancet of an individual, such as from a finger prick or a heel prick. In some embodiments, the absorbent or bibulous member comprises 1 to 10 delimited zoned configured for deposition of a blood sample. In some embodiments, the absorbent or bibulous member comprises 1 to 10 delimited zoned configured for deposition of a blood sample, wherein each delimited zone is configured to accept a blood sample having a volume of about 250 μL or less.

### II. Extraction Internal Standards and Uses Thereof

In certain aspects, provided herein are extraction internal standards suitable for use with the absorbent or bibulous members, such as a blood spot card, and methodology described herein. In some embodiments, provided is one or more extraction internal standards, wherein at least one of the one or more extraction internal standards comprises a polypeptide standard. In some embodiments, all of the one or more extraction internal standards are polypeptide standards. In some embodiments, a single extraction internal standard is used in the compositions and methods described herein, wherein the extraction internal standard is a polypeptide standard. In some embodiments, a plurality of extraction internal standards are used in the compositions and methods described herein, wherein at least one of the extraction internal standards is a polypeptide standard. As described herein, the polypeptide standards have a known composition, known amount, and known location on the absorbent or bibulous member (e.g., prior to deposition of a blood sample) such that the polypeptide standard serves as a reference point for one or more processes involved with analyzing the components of the blood sample using an LC-MS technique. In some embodiments, the polypeptide standard having a known location will move, such as due to placement of a blood sample on an absorbent or bibulous member. For example, as described in more detail herein, in the some embodiments, the polypeptide standard is useful for determining one or more of an extraction efficiency, a digestion efficiency, a sample migration pattern, or quantification of a polypeptide.

In some embodiments, the one or more extraction internal standards comprise a plurality of polypeptide standards, wherein at least two polypeptide standards of the plurality have different amino acid lengths. In some embodiments, the at least two polypeptide standards of the plurality are different in length by 1 or more amino acid, such as any of 5 or more, 10 or more, 20 or more, 30 or more, 40 or more, 50 or more, 60 or more, 70 or more, 80 or more, 90 or more, 100 or more, 150 or more, 200 or more, 250 or more, 300 or more, 400 or more, 500 or more, 600 or more, 700 or more, 800 or more, 900 or more, 1000 or more, 1100 or more, 1200 or more, 1300 or more, 1400 or more, 15000 or more, 1600 or more, 1700 or more, 1800 or more, 1900 or more, or 2000 or more amino acids. In some embodiments, the at least two polypeptide standards of the plurality are different in length by about any of 1, 5, 10, 20, 30, 40, 50, 60, 70, 80, 90, 100, 150, 200, 250, 300, 400, 500, 600, 700, 800, 900, 1000, 1100, 1200, 1300, 1400, 15000, 1600, 1700, 1800, 1900, or 2000 amino acids.

In some embodiments, the polypeptide standard has an amino acid length of at least about 4 amino acids to about 200 amino acids, such as any of about 5 amino acids to about 1900 amino acids, about 10 amino acids to about 1800 amino acids, about 20 amino acids to about 1700 amino acids, about 30 amino acids to about 1600 amino acids, about 40 amino acids to about 1500 amino acids, about 50 amino acids to about 1400 amino acids, about 60 amino acids to about 1300 amino acids, about 70 amino acids to about 1200 amino acids, about 80 amino acids to about 1100 amino acids, about 90 amino acids to about 1000 amino acids, about 100 amino acids to about 900 amino acids, about 200 amino acids to about 800 amino acids, about 300 amino acids to about 700 amino acids, about 400 amino acids to about 600 amino acids, or about 500 amino acids to about 750 amino acids. In some embodiments, the polypeptide standard has an amino acid length of at least about 4 amino acids, such as at least about any of 10 amino acids, 20 amino acids, 30 amino acids, 40 amino acids, 50 amino acids, 75 amino acids, 100 amino acids, 150 amino acids, 200 amino acids, 250 amino acids, 300 amino acids, 350 amino acids, 400 amino acids, 450 amino acids, 500 amino acids, 550 amino acids, 600 amino acids, 650 amino acids, 700 amino acids, 750 amino acids, 800 amino acids, 850 amino acids, 900 amino acids, 950 amino acids, 1000 amino acids, 1100 amino acids, 1200 amino acids, 1300 amino acids, 1400 amino acids, 1500 amino acids, 1600 amino acids, 1700 amino acids, 1800 amino acids, 1900 amino acids, or 2000 amino acids.

In some embodiments, at least one polypeptide standard of the one or more extraction internal standards comprises at least one internal enzymatic cleavage site, such as a cleavage site for trypsin or LysC. In some embodiments, the internal enzymatic cleavage site of the at least one polypeptide standard is cleaved during the provided method, resulting in two or more polypeptide fragments, wherein the two or more polypeptide fragments are shorter than the uncleaved polypeptide standard. In some embodiments, the cleaved polypeptide fragments comprise a C-terminal arginine or lysine. In some embodiments, the at least one polypeptide standard comprises a polypeptide comprising a C-terminal arginine or lysine.

In some embodiments, the one or more extraction internal standards comprise a plurality of polypeptide standards, wherein at least two of the plurality of polypeptide standards have different net hydrophobicities. In some embodiments, the net hydrophobicity is based on a partition coefficient analysis. In some embodiments, the net hydrophobicity is based on an octanol and water partition coefficient. In some embodiments, the net hydrophobicity is based on a computational tool. In some embodiments, the plurality of polypeptide standards having different net hydrophobicities comprises a hydrophobicity range. In some embodiments, the plurality of polypeptide standards comprise a hydrophobicity range as defined by the Grand average of hydropathicity index (GRAVY). In some embodiments, a hydrophobicity range as defined by GRAVY is about −0.5 to about 1. In some embodiments, a hydrophobicity range as defined by GRAVY is about −1 to about 2, about −0.8 to about 1.8, about −0.6 to about 1.6, about −0.4 to about 1.4, about −0.2 to about 1.2, about 0 to about 1, about 0.2 to about 0.8, about 0.4 to about 0.6, or about 0.5 to about 0.9. In some embodiments, a hydrophobicity range as defined by GRAVY is −1 to 2, −0.8 to 1.8, −0.6 to 1.6, −0.4 to 1.4, −0.2 to 1.2, 0 to 1, 0.2 to 0.8, 0.4 to 0.6, or 0.5 to 0.9. In some embodiments, a hydrophobicity range as defined by GRAVY is −0.5 to 1.

In some embodiments, the at least one polypeptide standard of the one or more extraction internal standards comprises a sequence that is non-homologous to an endogenous polypeptide of an individual from which the blood sample originates. In some embodiments, the at least one polypeptide standard comprises an amino acid sequence that does not have homology to a peptide derived from the human proteome. In some embodiments, the at least one polypeptide standard comprises an amino acid sequence derived from a bacterial, a fungal, an insect, a plant, or a non-human animal proteome. In some embodiments, the at least one polypeptide standard comprises an amino acid sequence of unknown origin (e.g., a sequence not found in known organisms or exogenous).

In some embodiments, the at least one polypeptide standard of the one or more extraction internal standards is a synthetic polypeptide. In some embodiments, the synthetic peptide is one or more of SEQ ID NOS: 21 and 22. In some embodiments, the at least one polypeptide standard comprises a sequence that is an analog of an endogenous polypeptide of an individual from which the blood sample originates. In some embodiments, the at least one polypeptide standard comprises an unnatural amino acid. In some embodiments, the unnatural amino acid comprises a fluorescent moiety, a functional moiety, and/or a reactive moiety.

In some embodiments, the at least one polypeptide standard of the one or more extraction internal standards is a synthetic polypeptide, wherein the at least one polypeptide standard comprises a stable heavy isotope label. In some embodiments, the stable heavy isotope label of the at least one polypeptide standard comprises one or more heavy labeled arginine, lysine, leucine, valine, or a combination thereof. In some embodiments, the stable heavy isotope label of the at least one polypeptide standard comprises 2-plex labeling, 3-plex labeling, or higher multiplex labeling. In some embodiments, the stable heavy isotope label of the at least one polypeptide standard is produced using stable isotope labeling by amino acids in cell culture (SILAC). In some embodiments, the analog of the endogenous polypeptide is a stable heavy isotope labeled analog polypeptide. In some embodiments, the non-homologous polypeptide is a stable heavy isotope labeled non-homologous polypeptide. In some embodiments, the non-human polypeptide is a stable heavy isotope labeled non-human polypeptide.

In some embodiments, the at least one polypeptide standard of the one or more extraction internal standards is a recombinantly expressed polypeptide. In some embodiments, the recombinantly expressed polypeptide comprises an amino acid sequence range from about 4 amino acids to about 2000 amino acids, about 5 amino acids to about 1900 amino acids, about 10 amino acids to about 1800 amino acids, about 20 amino acids to about 1700 amino acids, about 30 amino acids to about 1600 amino acids, about 40 amino acids to about 1500 amino acids, about 50 amino acids to about 1400 amino acids, about 60 amino acids to about 1300 amino acids, about 70 amino acids to about 1200 amino acids, about 80 amino acids to about 1100 amino acids, about 90 amino acids to about 1000 amino acids, about 100 amino acids to about 900 amino acids, about 200 amino acids to about 800 amino acids, about 300 amino acids to about 700 amino acids, about 400 amino acids to about 600 amino acids, or about 500 amino acids to about 750 amino acids. In some embodiments, the recombinantly expressed polypeptide comprises at least one internal enzymatic cleavage site. In some embodiments, the recombinantly expressed polypeptide of the one or more extraction internal standards have different net hydrophobicities. In some embodiments, the recombinantly expressed polypeptide comprises a sequence that is non-homologous to an endogenous polypeptide of an individual from which the blood sample originates. In some embodiments, the recombinantly expressed polypeptide comprises a sequence that does not have homology to a peptide derived from the human proteome. In some embodiments, the recombinantly expressed polypeptide comprises an analog sequence, wherein one or more amino acids are unnatural or derivatives of the endogenous sequence. In some embodiments, the recombinantly expressed polypeptide is a synthetic polypeptide. In some embodiments, the recombinantly expressed polypeptide comprises a stable heavy isotope label.

In some embodiments, the at least one polypeptide standard of the one or more extraction internal standards is a glycopolypeptide. In some embodiments, the glycopolypeptide comprises an amino acid sequence range from about 4 amino acids to about 2000 amino acids, about 5 amino acids to about 1900 amino acids, about 10 amino acids to about 1800 amino acids, about 20 amino acids to about 1700 amino acids, about 30 amino acids to about 1600 amino acids, about 40 amino acids to about 1500 amino acids, about 50 amino acids to about 1400 amino acids, about 60 amino acids to about 1300 amino acids, about 70 amino acids to about 1200 amino acids, about 80 amino acids to about 1100 amino acids, about 90 amino acids to about 1000 amino acids, about 100 amino acids to about 900 amino acids, about 200 amino acids to about 800 amino acids, about 300 amino acids to about 700 amino acids, about 400 amino acids to about 600 amino acids, or about 500 amino acids to about 750 amino acids. In some embodiments, the glycopolypeptide comprises at least one internal enzymatic cleavage site. In some embodiments, the glycopolypeptide of the one or more extraction internal standards have different net hydrophobicities. In some embodiments, the glycopolypeptide comprises a sequence that is non-homologous to an endogenous polypeptide of an individual from which the blood sample originates. In some embodiments, the glycopolypeptide comprises a sequence that does not have homology to a peptide derived from the human proteome. In some embodiments, the glycopolypeptide comprises an analog sequence, wherein one or more amino acids are unnatural or derivatives of the endogenous sequence. In some embodiments, the glycopolypeptide is a synthetic polypeptide. In some embodiments, the glycopolypeptide comprises a stable heavy isotope label.

In some embodiments, the at least one polypeptide standard of the one or more extraction internal standards is a polypeptide that does not substantially interact with hemoglobin. In some embodiments, hemoglobin does not substantially impact the extraction of the polypeptide. In some embodiments, hemoglobin does not substantially impact the processing of the polypeptide. In some embodiments, hemoglobin does not substantially impact the digestion of the polypeptide. I

In some embodiments, the at least one polypeptide standard of the one or more extraction internal standards comprises a contiguous sequence from SEQ ID NOS: 14-20 as shown in Table 7. In some embodiments, the at least one polypeptide standard of the one or more extraction internal standards comprises at least a contiguous 4 amino acid sequence from SEQ ID NOS: 14-20. In some embodiments, the at least one polypeptide standard of the one or more extraction internal standards comprises at least a contiguous sequence of about 4, about 5, about 6, about 7, about 8, about 9, about 10, about 11, about 12, about 13, about 14, about 15, about 16, about 17, about 18, about 19, about 20, about 30, about 40, about 50, about 60, about 70, about 80, about 90, or about 100 acids from SEQ ID NOS: 14-20. In some embodiments, the at least one polypeptide standard of the one or more extraction internal standards comprises one or more polypeptides human apolipoprotein C-III (APOC3) of SEQ ID NO: 14, human alpha-2-macroglobulin (A2MG) of SEQ ID NO: 15, human ceruloplasmin (CERU) of SEQ ID NO: 16, human alpha-1-acid glycoprotein 1 (AGP1) of SEQ ID NO: 17, human haptoglobin (HPT) of SEQ ID NO: 18, human hemopexin (HEMO) of SEQ ID NO: 19, or human beta-2-glycoprotein 1 (APOH) of SEQ ID NO: 20. In some embodiments, the at least one polypeptide standard of the one or more extraction internal standards is APOC3, for example SEQ ID NO: 14. In some embodiments, the at least one polypeptide standard of the one or more extraction internal standards is A2MG, for example SEQ ID NO: 15. In some embodiments, the at least one polypeptide standard of the one or more extraction internal standards is CERU, for example SEQ ID NO: 16. In some embodiments, the at least one polypeptide standard of the one or more extraction internal standards is AGP1, for example SEQ ID NO: 17. In some embodiments, the at least one polypeptide standard of the one or more extraction internal standards is HPT, for example SEQ ID NO: 18. In some embodiments, the at least one polypeptide standard of the one or more extraction internal standards is HEMO, for example SEQ ID NO: 19. In some embodiments, the at least one polypeptide standard of the one or more extraction internal standards is APOH, for example SEQ ID NO: 20. In some embodiments, the at least one polypeptide standard of the one or more extraction internal standards has at least 85%, at least 90%, at least 95%, at least 96%, at least 97%, at least 98%, or at least 99% sequence identity to one or more of SEQ ID NOS: 14-20. In some embodiments, the at least one polypeptide standard of the one or more extraction internal standards has 85%, 86%, 87%, 88%, 89%, 90%, 91%, 92%, 93%, 94%, 95%, 96%, 97%, 98%, or 99% sequence identity to one or more of SEQ ID NOS: 14-20. In some embodiments, the at least one polypeptide standard of the one or more extraction internal standards is any of the polypeptides described herein, wherein the polypeptide further comprises one or more of an amino acid sequence range from about 4 amino acids to about 2000 amino acids, at least one internal enzymatic cleavage site, a unique net hydrophobicity, a sequence that is non-homologous to an endogenous polypeptide of an individual from which the blood sample originates, a sequence that does not have homology to a peptide derived from the human proteome, an analog sequence, wherein one or more amino acids are unnatural or derivatives of the endogenous sequence, a stable heavy isotope label, a polypeptide that does not substantially interact with hemoglobin, a recombinantly expressed polypeptide, or a glycopolypeptide. In an embodiment, the proteins of SEQ ID NOS: 14-20 are heavy labeled proteins where all arginines and lysines are replaced with stable isotope labeled lysine and arginine. The proteins are labeled with a stable isotopes so that they can be monitored without confusing them for the endogenous proteins in the blood sample. Such heavy labeled glycoproteins could be produced through recombinant expression in an orthologous system, cultured in SILAC supplemented media. In an embodiment, the proteins of SEQ ID NOS: 14-20 have all of their lysines and arginines heavy isotope labeled and deposited on a filter card. When blood is applied, a portion of the labeled proteins of SEQ ID NOS: 14-20 is extracted from the filter paper or DBS and then digested with a proteolytic enzyme. A resulting peptide produced from the enzymatic digestion having the isotope label from each of the proteins of SEQ ID NOS: 14-20 can be monitored with multiple reaction monitoring (MRM) for determining the extraction efficiency and/or tryptic efficiency for each of the labeled proteins. The labeled isotope allows the trypic peptides generated from the proteins of SEQ ID NOS: 14-20 to be distinguished from naturally occurring tryptic peptides. In an embodiment, peptides of SEQ ID NOS: 21 and 22 can represent an exogenous or non-human peptide not normally found in human subjects and be deposited on a filter paper or DBS as shown in Table 7. Since SEQ ID NOS: 21 and 22 are not normally found in human subjects, they can be used for determining the extraction efficiency.

TABLE 7
Sources of example polypeptide standards.

| SEQ | | |
|---|---|---|
| ID | | |
| NO: | Sequence | Origin |
| 114 | MQPRVLLVVALLALLASARASEAEDASLLSFMQGY | Human |
| | MKHATKTAKDALSSVQESQVAQQARGWVTDGFSSL | Apolipoprotein |
| | KDYWSTVKDKFSEFWDLDPEVRPTSAVAA | C-III |
| | | (APOC3) |
| 215 | MGKNKLLHPSLVLLLLVLLPTDASVSGKPQYMVLV | Human |
| | PSLLHTETTEKGCVLLSYLNETVTVSASLESVRGN | Alpha-2- |
| | RSLFTDLEAENDVLHCVAFAVPKSSSNEEVMFLTV | macroglobulin |
| | QVKGPTQEFKKRTTVMVKNEDSLVFVQTDKSIYKP | (A2MG) |
| | GQTVKFRVVSMDENFHPLNELIPLVYIQDPKGNRI | |
| | AQWQSFQLEGGLKQFSFPLSSEPFQGSYKVVVQKK | |
| | SGGRTEHPFTVEEFVLPKFEVQVTVPKIITILEEE | |
| | MNVSVCGLYTYGKPVPGHVTVSICRKYSDASDCHG | |
| | EDSQAFCEKFSGQLNSHGCFYQQVKTKVFQLKRKE | |
| | YEMKLHTEAQIQEEGTVVELTGRQSSEITRTITKL | |
| | SFVKVDSHFRQGIPFFGQVRLVDGKGVPIPNKVIF | |
| | IRGNEANYYSNATTDEHGLVQFSINTTNVMGTSLT | |
| | VRVNYKDRSPCYGYQWVSEEHEEAHHTAYLVFSPS | |
| | KSFVHLEPMSHELPCGHTQTVQAHYILNGGTLLGL | |
| | KKLSFYYLIMAKGGIVRTGTHGLLVKQEDMKGHFS | |
| | ISIPVKSDIAPVARLLIYAVLPTGDVIGDSAKYDV | |
| | ENCLANKVDLSFSPSQSLPASHAHLRVTAAPQSVC | |
| | ALRAVDQSVLLMKPDAELSASSVYNLLPEKDLTGF | |
| | PGPLNDQDNEDCINRHNVYINGITYTPVSSTNEKD | |
| | MYSFLEDMGLKAFTNSKIRKPKMCPQLQQYEMHGP | |
| | EGLRVGFYESDVMGRGHARLVHVEEPHTETVRKYF | |
| | PETWIWDLVVVNSAGVAEVGVTVPDTITEWKAGAF | |
| | CLSEDAGLGISSTASLRAFQPFFVELTMPYSVIRG | |
| | EAFTLKATVLNYLPKCIRVSVQLEASPAFLAVPVE | |
| | KEQAPHCICANGRQTVSWAVTPKSLGNVNFTVSAE | |
| | ALESQELCGTEVPSVPEHGRKDTVIKPLLVEPEGL | |
| | EKETTFNSLLCPSGGEVSEELSLKLPPNVVEESAR | |
| | ASVSVLGDILGSAMQNTQNLLQMPYGCGEQNMVLF | |
| | APNIYVLDYLNETQQLTPEIKSKAIGYLNTGYQRQ | |
| | LNYKHYDGSYSTFGERYGRNQGNTWLTAFVLKTFA | |
| | QARAYIFIDEAHITQALIWLSQRQKDNGCFRSSGS | |
| | LLNNAIKGGVEDEVTLSAYITIALLEIPLTVTHPV | |
| | VRNALFCLESAWKTAQEGDHGSHVYTKALLAYAFA | |
| | LAGNQDKRKEVLKSLNEEAVKKDNSVHWERPQKPK | |
| | APVGHFYEPQAPSAEVEMTSYVLLAYLTAQPAPTS | |
| | EDLTSATNIVKWITKQQNAQGGFSSTQDTVVALHA | |
| | LSKYGAATFTRTGKAAQVTIQSSGTFSSKFQVDNN | |
| | NRLLLQQVSLPELPGEYSMKVTGEGCVYLQTSLKY | |
| | NILPEKEEFPFALGVQTLPQTCDEPKAHTSFQISL | |
| | SVSYTGSRSASNMAIVDVKMVSGFIPLKPTVKMLE | |
| | RSNHVSRTEVSSNHVLIYLDKVSNQTLSLFFTVLQ | |
| | DVPVRDLKPAIVKVYDYYETDEFAIAEYNAPCSKD | |
| | LGNA | |
| 316 | MKILILGIFLFLCSTPAWAKEKHYYIGIIETTWDY | Human |
| | ASDHGEKKLISVDTEHSNIYLQNGPDRIGRLYKKA | ceruloplasmin |
| | LYLQYTDETFRTTIEKPVWLGFLGPIIKAETGDKV | (CERU) |
| | YVHLKNLASRPYTFHSHGITYYKEHEGAIYPDNTT | |
| | DFQRADDKVYPGEQYTYMLLATEEQSPGEGDGNCV | |
| | TRIYHSHIDAPKDIASGLIGPLIICKKDSLDKEKE | |
| | KHIDREFVVMFSVVDENFSWYLEDNIKTYCSEPEK | |
| | VDKDNEDFQESNRMYSVNGYTFGSLPGLSMCAEDR | |
| | VKWYLFGMGNEVDVHAAFFHGQALTNKNYRIDTIN | |
| | LFPATLFDAYMVAQNPGEWMLSCQNLNHLKAGLQA | |
| | FFQVQECNKSSSKDNIRGKHVRHYYIAAEEIIWNY | |
| | APSGIDIFTKENLTAPGSDSAVFFEQGTTRIGGSY | |
| | KKLVYREYTDASFTNRKERGPEEEHLGILGPVIWA | |
| | EVGDTIRVTFHNKGAYPLSIEPIGVRFNKNNEGTY | |
| | YSPNYNPQSRSVPPSASHVAPTETFTYEWTVPKEV | |
| | GPTNADPVCLAKMYYSAVDPTKDIFTGLIGPMKIC | |
| | KKGSLHANGRQKDVDKEFYLFPTVFDENESLLLED | |
| | NIRMFTTAPDQVDKEDEDFQESNKMHSMNGFMYGN | |
| | QPGLTMCKGDSVVWYLFSAGNEADVHGIYFSGNTY | |
| | LWRGERRDTANLFPQTSLTLHMWPDTEGTFNVECL | |
| | TTDHYTGGMKQKYTVNQCRRQSEDSTFYLGERTYY | |
| | IAAVEVEWDYSPQREWEKELHHLQEQNVSNAFLDK | |
| | GEFYIGSKYKKVVYRQYTDSTFRVPVERKAEEEHL | |
| | GILGPQLHADVGDKVKIIFKNMATRPYSIHAHGVQ | |
| | TESSTVTPTLPGETLTYVWKIPERSGAGTEDSACI | |
| | PWAYYSTVDQVKDLYSGLIGPLIVCRRPYLKVFNP | |
| | RRKLEFALLFLVFDENESWYLDDNIKTYSDHPEKV | |
| | NKDDEEFIESNKMHAINGRMFGNLQGLTMHVGDEV | |
| | NWYLMGMGNEIDLHTVHFHGHSFQYKHRGVYSSDV | |
| | FDIFPGTYQTLEMFPRTPGIWLLHCHVTDHIHAGM | |
| | ETTYTVLQNEDTKSG | |
| 174 | MALSWVLTVLSLLPLLEAQIPLCANLVPVPITNAT | Human |
| | LDQITGKWFYIASAFRNEEYNKSVQEIQATFFYFT | Alpha-1- |
| | PNKTEDTIFLREYQTRQDQCIYNTTYLNVQRENGT | acid |
| | ISRYVGGQEHFAHLLILRDTKTYMLAFDVNDEKNW | glycoprotein 1 |
| | GLSVYADKPETTKEQLGEFYEALDCLRIPKSDVVY | (AGP1) |
| | TDWKKDKCEPLEKQHEKERKQEEGES | |
| 185 | MSALGAVIALLLWGQLFAVDSGNDVTDIADDGCPK | Human |
| | PPEIAHGYVEHSVRYQCKNYYKLRTEGDGVYTLND | Haptoglobin |
| | KKQWINKAVGDKLPECEADDGCPKPPEIAHGYVEH | (HPT) |
| | SVRYQCKNYYKLRTEGDGVYTLNNEKQWINKAVGD | |
| | KLPECEAVCGKPKNPANPVQRILGGHLDAKGSFPW | |
| | QAKMVSHHNLTTGATLINEQWLLTTAKNLFLNHSE | |
| | NATAKDIAPTLTLYVGKKQLVEIEKVVLHPNYSQV | |
| | DIGLIKLKQKVSVNERVMPICLPSKDYAEVGRVGY | |
| | VSGWGRNANFKFTDHLKYVMLPVADQDQCIRHYEG | |
| | STVPEKKTPKSPVGVQPILNEHTFCAGMSKYQEDT | |
| | CYGDAGSAFAVHDLEEDTWYATGILSFDKSCAVAE | |
| | YGVYVKVTSIQDWVQKTIAEN | |
| 196 | MARVLGAPVALGLWSLCWSLAIATPLPPTSAHGNV | Human |
| | AEGETKPDPDVTERCSDGWSFDATTLDDNGTMLFF | Hemopexin |
| | KGEFVWKSHKWDRELISERWKNFPSPVDAAFRQGH | (HEMO) |
| | NSVFLIKGDKVWVYPPEKKEKGYPKLLQDEFPGIP | |
| | SPLDAAVECHRGECQAEGVLFFQGDREWFWDLATG | |
| | TMKERSWPAVGNCSSALRWLGRYYCFQGNQFLRFD | |
| | PVRGEVPPRYPRDVRDYFMPCPGRGHGHRNGTGHG | |
| | NSTHHGPEYMRCSPHLVLSALTSDNHGATYAFSGT | |
| | HYWRLDTSRDGWHSWPIAHQWPQGPSAVDAAFSWE | |
| | EKLYLVQGTQVYVFLTKGGYTLVSGYPKRLEKEVG | |
| | TPHGIILDSVDAAFICPGSSRLHIMAGRRLWWLDL | |
| | KSGAQATWTELPWPHEKVDGALCMEKSLGPNSCSA | |
| | NGPGLYLIHGPNLYCYSDVEKLNAAKALPQPQNVT | |
| | SLLGCTH | |
| 207 | MISPVLILFSSFLCHVAIAGRTCPKPDDLPFSTVV | Human |
| | PLKTFYEPGEEITYSCKPGYVSRGGMRKFICPLTG | Beta-2- |
| | LWPINTLKCTPRVCPFAGILENGAVRYTTFEYPNT | glycoprotein 1 |
| | ISFSCNTGFYLNGADSAKCTEEGKWSPELPVCAPI | (APOH) |
| | ICPPPSIPTFATLRVYKPSAGNNSLYRDTAVFECL | |
| | PQHAMFGNDTITCTTHGNWTKLPECREVKCPFPSR | |
| | PDNGFVNYPAKPTLYYKDKATFGCHDGYSLDGPEE | |
| | IECTKLGNWSAMPSCKASCKVPVKKATVVYQGERV | |
| | KIQEKFKNGMLHGDKVSFFCKNKEKKCSYTEDAQC | |
| | IDGTIEVPKCFKEHSSLAFWKTDASDVKPC | |
| 218 | RPAIAINNPYVPR | |
| 229 | FFVAPFPEVFGK | |

In some embodiments, the at least one polypeptide standard of the one or more extraction internal standards is present in a known amount on the absorbent or bibulous member, such as a blood spot card. In some embodiments, the at least one polypeptide standard is present in a known amount on the absorbent or bibulous member, such as a blood spot card, prior to contact the absorbent or bibulous member with a blood sample. In some embodiments, the known amount of each of the one or more extraction internal standards, such as a polypeptide standard, is about 0.05 ppm to about 5 ppm. In some embodiments, the known amount of each of the one or more extraction internal standards, such as a polypeptide standard, is about 0.02 ppm to about 10 ppm, about 0.05 ppm to about 9 ppm, about 0.1 ppm to about 9 ppm, about 0.2 ppm to about 8 ppm, about 0.3 ppm to about 7 ppm, about 0.4 ppm to about 6 ppm, about 0.5 ppm to about 5 ppm, about 0.6 ppm to about 4 ppm, about 0.7 ppm to about 3 ppm, about 0.8 ppm to about 2 ppm, or about 0.9 ppm to about 1 ppm.

In some embodiments, the one or more extraction internal standards are deposited and dried on the absorbent or bibulous member, such as a blood spot card, within an area having a surface area of about 1,000 mm² or less. In some embodiments, the area for depositing the one or more extraction internal standards, such as a polypeptide standard, has a surface area of about 1,000 mm² or less, about 950 mm² or less, about 900 mm² or less, about 850 mm² or less, about 800 mm² or less, about 750 mm² or less, about 700 mm² or less, about 650 mm² or less, about 600 mm² or less, about 550 mm² or less, about 500 mm² or less, about 450 mm² or less, about 400 mm² or less, about 350 mm² or less, about 300 mm² or less, about 250 mm² or less, about 200 mm² or less, about 150 mm² or less, about 100 mm² or less, or about 50 mm² or less. In some embodiments, the area for depositing the one or more extraction internal standards, such as a polypeptide standard, has a surface area of 1,000 mm² or less, 950 mm² or less, 900 mm² or less, 850 mm² or less, 800 mm² or less, 750 mm² or less, 700 mm² or less, 650 mm² or less, 600 mm² or less, 550 mm² or less, 500 mm² or less, 450 mm² or less, 400 mm² or less, 350 mm² or less, 300 mm² or less, 250 mm² or less, 200 mm² or less, 150 mm² or less, 100 mm² or less, or 50 mm² or less. In some embodiments, the less can represent the lower limit of a range for surface area as described above where the lower limit is about 2 mm², 4 mm², 6 mm², 8 mm², or 10 mm².

In some embodiments, the one or more extraction internal standard, such as a polypeptide standard, are deposited and dried on the absorbent or bibulous member, such as a blood spot card, within the delimited zone. In some embodiments, the delimited zone for depositing and drying the one or more extraction internal standard is a defined area, such as marked by a visible line or dash. In some embodiments, the delimited zone is about 1,000 mm² or less. In some embodiments, the delimited zone is any size described herein. In some embodiments, the delimited zone contains a known amount of each of the one or more extraction internal standards, such as a polypeptide standard. In some embodiments, the delimited zone contains about 0.05 ppm to about 5 ppm of each of the one or more extraction internal standards.

III. Methods Associated with the Absorbent or Bibulous Members Described Herein

In certain aspects, provided herein are methods associated with the absorbent or bibulous members described herein, including absorbent or bibulous members comprising one or more extraction internal standards, wherein at least one of the extraction internal standards is a polypeptide standard.

In some embodiments, provided is a method of making an absorbent or bibulous member described herein, such as a blood spot card, comprising one or more extraction internal standards, wherein at least one of the extraction internal standards is a polypeptide standard, the method comprising depositing the one or more extraction internal standards on the absorbent or bibulous member. In some embodiments, the method comprises drying the one or more extraction internal standards following deposition on the absorbent or bibulous member.

In some embodiments, provided is a method for obtaining an absorbent or bibulous member, such as a blood spot card, comprising a blood sample deposited thereon, the method comprising providing the absorbent or bibulous member comprising one or more extraction internal standards, wherein at least one of the extraction internal standards is a polypeptide standard, and providing instructions for the deposition of the blood sample onto the absorbent or bibulous member. In some embodiments, the method comprises depositing the blood sample onto the absorbent or bibulous member. In some embodiments, the method comprises mailing, such as via standard mail with a government regulated mail carrier, e.g., The United States Postal Service, the absorbent or bibulous member comprising the blood sample and the one or more extraction internal standards.

In some embodiments, provided is a method of extracting at least a portion of a plurality of polypeptides and one or more extraction internal standards, wherein the one or more extraction internal standards comprises a polypeptide standard, from an absorbent or bibulous member, such as a blood spot card. In some embodiments, the method comprises: separating one or more portions of the blood spot card from the blood spot card, wherein the one or more portions of the absorbent or bibulous member comprise at least a portion of the blood sample and the one or more extraction internal standards; extracting at least the portion of the plurality of polypeptides and the one or more extraction internal standards from the one or more portions of the absorbent or bibulous member into an extraction solution; and precipitating at least the portion of the plurality of polypeptides and the one or more extraction internal standards to obtain the extracted sample. In some embodiments, the separating the one or more portions of the absorbent or bibulous member comprises punching the one or more portion of the absorbent or bibulous member using a punching device. In some embodiments, the separated portion of the absorbent or bibulous member is referred to as a chad. In some embodiments, the extracting may include a thermal denaturation step and as water bath sonication in order to mix and promote diffusion of glycoproteins out of absorbent or bibulous member and into the extraction solution. The extraction solution may include buffer such as 50 mM ammonium bicarbonate, and a reducing agent such as 25 mM DTT to help promote denaturation and extraction of the proteins.

In some embodiments, each of the one or more portions separated from the absorbent or bibulous member have a surface area of about 2 mm² to about 100 mm². In some embodiments, each of the one or more portions separated from the absorbent or bibulous member have a surface area of about any of 1 mm², 2 mm², 3 mm², 4 mm², 5 mm², 6 mm², 7 mm², 8 mm², 9 mm², 10 mm², 15 mm², 20 mm², 25 mm², 30 mm², 35 mm², 40 mm², 45 mm², 50 mm², 55 mm², 60 mm², 65 mm², 70 mm², 75 mm², 80 mm², 85 mm², 90 mm², 95 mm², or 100 mm².

In some embodiments, the precipitating at least the portion of the plurality of polypeptides and the one or more extraction internal standards comprises subjecting the at least the portion of the plurality of polypeptides and the one or more extraction internal standards to an organic solvent, such as ethanol.

In some embodiments, the method further comprises adding a solution, such as a buffer, e.g., ammonium bicarbonate, to the extracted sample to resolubilize polypeptide content therein prior to subjecting the extracted sample or the derivative thereof to the proteolytic digestion technique.

In some embodiments, provided herein is a method for performing a liquid chromatography-mass spectrometry (LC-MS) analysis of a proteolytic glycopeptide derived from a blood sample deposited on a delimited zone of an absorbent or bibulous member, such as a blood spot card, wherein the blood sample comprises a plurality of polypeptides comprising at least one glycoprotein, the method comprising: extracting at least a portion of the plurality of polypeptides and one or more extraction internal standards from the absorbent or bibulous member to obtain an extracted sample, wherein the absorbent or bibulous member comprises the one or more extraction internal standards prior to deposition of the blood sample within the delimited zone, and wherein at least one of the one or more extraction internal standards comprises a polypeptide standard; subjecting the extracted sample or a derivative thereof to a proteolytic digestion technique to produce a proteolytically digested sample comprising the proteolytic glycopeptide; introducing at least a portion of the proteolytically digested sample to a liquid chromatography (LC) system of a LC-MS system; and performing the LC-MS analysis on at least the proteolytic glycopeptide and the one or more extraction internal standards.

In some embodiments, the performing the LC-MS analysis comprises measuring an abundance signal for the proteolytic glycopeptide and an abundance signal for the one or more extraction internal standards. In some embodiments, the performing the LC-MS analysis further comprises calculating a concentration of the proteolytic glycopeptide based on a concentration of the one or more extraction internal standards prior to deposition on the absorbent or bibulous member, such as a blood spot card, the abundance signal for the proteolytic glycopeptide, and the abundance signal for the one or more extraction internal standards.

In some embodiments, the method further comprises determining an extraction efficiency based on the LC-MS analysis of at least one of the one or more extraction internal standards. For example, in some embodiments, the extraction efficiency is based on an abundance signal for the one or more extraction internal standards as compared to a reference, such as a known amount of the one or more extraction internal standards deposited on the absorbent or bibulous member and/or comparison of two or more of the extraction internal standards.

In some embodiments, the method further comprises determining a digestion efficiency based on the LC-MS analysis of at least one of the one or more extraction internal standards. For example, in some embodiments, the digestion efficiency is based on an abundance signal for the one or more extraction internal standards, such as depletion of an abundance signal of a polypeptide standard comprising a protease cleavage site, and/or an increase in an abundance signal for one or more portions of a polypeptide standard, wherein the one or more portions of the polypeptide standard are a result of protease activity.

In some embodiments, the method further comprises assessing a sample migration pattern based on the LC-MS analysis of at least one of the one or more extraction internal standards. For example, in some embodiments, the sample migration pattern is based on an abundance signal for the one or more extraction internal standards obtained from a delimited zone of an absorbent or bibulous member, wherein the one or more extraction internal standards have known location prior to deposition of a blood sample on the absorbent or bibulous member. In some embodiments, the known location of the one or more extraction internal standards is within the delimited zone and the sample migration pattern provides information regarding the movement of the one or more extraction internal standards upon deposition of a blood sample. In some embodiments, the known location of the one or more extraction internal standards is outside of the delimited zone and the sample migration pattern provides information regarding the movement of the one or more extraction internal standards to the delimited zone upon deposition of a blood sample. In some embodiments, the movement of the one or more extraction internal standards provides information regarding the movement of a blood sample, or a component thereof, after being deposited onto an absorbent or bibulous member.

### D3. Methods for Proteolytically Digesting a Blood Sample Comprising a Glycoprotein

In certain aspects, provided herein are methods of proteolytically digesting a biological sample, such as an extract from a dried blood sample, comprising a glycoprotein. A dried blood spot sample can be punched to separate one or more discs which are then soaked in a buffer containing DTT at an elevated temperature of 95° C. and sonicated to extract the proteins and glycoproteins. In some embodiments, the extract can be in the form of a precipitate after the addition of ethanol. In some embodiments, the methods comprising subjecting the extract from the dried blood sample, or a portion thereof, to another thermal denaturation technique. Proteases are enzymes that cleave polypeptides at, generally, specific cleavage motifs. For example, trypsin is a serine protease that generally cleaves polypeptides at the carboxyl side (C-terminal side) of lysine and arginine residues. A glycan of a glycopeptide may present a steric hindrance to a protease, thereby inhibiting complete protease digestion of the extract from the dried blood sample, or a portion thereof, comprising a glycoprotein. Without being bound to this theory, it is believed that the methods taught herein improve polypeptide unfolding, such as linearization, and provide protease access to cleavage sites thereby providing methods for more complete proteolytic digestion of glycoproteins.

In some aspects, provided is a method comprising subjecting the extract from the dried blood sample, or portion thereof, to a thermal denaturation technique to produce a denatured sample.

In other aspects, provided is a method comprising subjecting the extract from the dried blood sample, or portion thereof, to a thermal denaturation technique to produce a denatured sample followed by a proteolytic digestion technique to produce a proteolytically digested sample comprising a proteolytic glycopeptide. In some embodiments, the method comprises quenching one or more proteases used in a proteolytic digestion technique prior to a downstream technique, such as LC-MS.

In other aspects, provided herein is a method comprising: subjecting the extract from the dried blood sample, or portion thereof, to a thermal denaturation technique to produce a denatured sample; subjecting the denatured sample to a reduction technique to produce a reduced sample; subjecting the reduced sample to an alkylation technique to produce an alkylated sample; and subjecting the alkylated sample to a proteolytic digestion technique to produce a proteolytically digested sample comprising the proteolytic glycopeptide. In some embodiments, the method comprises quenching an alkylating agent used in the alkylation technique prior to subjecting an alkylated sample to a proteolytic digestion technique. In some embodiments, the method comprises quenching one or more proteases used in a proteolytic digestion technique prior to a downstream technique, such as LC-MS.

In some embodiments, the proteolytic digestion methods described herein produce a proteolytic digestion sample, including one comprising a proteolytic glycopeptide, having a digestion completion rate of at least about 70%, such as at least about any of 75%, 80%, 85%, 90%, 91%, 92%, 93%, 94%, 95%, 96%, 97%, 98%, or 99%. In some embodiments, the proteolytic digestion methods described herein produce a proteolytic digestion sample, including one comprising a proteolytic glycopeptide, wherein the sample volume loss is 10% or less, such as 9% or less, 8% or less, 7% or less, 6% or less, 5% or less, 4% or less, 3% or less, 2% or less, or 1% or less, based on all volumes added in producing the proteolytic digestion sample.

In the following sections, additional description of the various aspects of the proteolytic digestion techniques is provided. Such description in a modular fashion is not intended to limit the scope of the disclosure, and based on the teachings provided herein one of ordinary skill in the art will readily appreciate that certain modules can be integrated, at least in part. The section heading used herein are for organizational purposes only and are not to be construed as limiting the subject matter described.

### I. Thermal Denaturation Techniques

In certain aspects, the methods provided herein comprise performing a thermal denaturation technique. Thermal denaturation techniques, generally speaking, change certain polypeptides conformational structures, such as by unfolding and/or linearizing a polypeptide, to enable protease access to cleavage sites. Thermal denaturation techniques described herein comprise subjecting a sample, or a derivative thereof (e.g., a sample diluted with a buffer), to a thermal treatment of about 60° C. to about 100° C. for thermal denaturation incubation time of at least about 1 minute. In some embodiments, the thermal denaturation technique is not performed concurrently with a chemical denaturation technique, such as using high concentrations of denaturing agent, e.g., 6M urea. In some embodiments, the method does not include use of a chemical denaturation technique.

In some embodiments, the thermal denaturation incubation time is performed at a temperature of about 60° C. to about 100° C., such as any of about 70° C. to about 100° C., about 80° C. to about 100° C., about 90° C. to about 100° C., about 95° C. to about 100° C., or about 85° C. to about 95° C. In some embodiments, the thermal denaturation incubation time is performed at a temperature of at least about 60° C., such as at least about any of 65° C., 70° C., 75° C., 80° C., 85° C., 90° C., 95° C., or 100° C. In some embodiments, the thermal denaturation incubation time is performed at a temperature of about 100° C. or less, such as about any of 95° C. or less, 90° C. or less, 85° C. or less, 80° C. or less, 75° C. or less, 70° C. or less, 65° C. or less, 60° C. or less. In some embodiments, the thermal denaturation incubation time is performed at a temperature of about any of 60° C., 65° C., 70° C., 75° C., 80° C., 85° C., 90° C., 91° C., 92° C., 93° C., 94° C., 95° C., 96° C., 97° C., 98° C., 99° C., or 100° C.

In some embodiments, the thermal denaturation incubation time is about 1 minute to about 15 minutes, such as any of about 1 minute to about 5 minutes, about 1 minute to about 10 minutes, about 2.5 minutes to about 7.5 minutes, or about 5 minutes to about 15 minutes. In some embodiments, the thermal denaturation incubation time is at least about 1 minute, such as at least about any of 1.5 minutes, 2 minutes, 2.5 minutes, 3 minutes, 3.5 minutes, 4 minutes, 4.5 minutes, 5 minutes, 5.5 minutes, 6 minutes, 6.5 minutes, 7 minutes, 7.5 minutes, 8 minutes, 8.5 minutes, 9 minutes, 9.5 minutes, 10 minutes, 11 minutes, 12 minutes, 13 minutes, 14 minutes, or 15 minutes. In some embodiments, the thermal denaturation incubation time is about 15 minutes or less, such as about any of 14 minutes or less, 13 minutes or less, 12 minutes or less, 11 minutes or less, 10 minutes or less, 9.5 minutes or less, 9 minutes or less, 8.5 minutes or less, 8 minutes or less, 7.5 minutes or less, 7 minutes or less, 6.5 minutes or less, 6 minutes or less, 5.5 minutes or less, 5 minutes or less, 4.5 minutes or less, 4 minutes or less, 3.5 minutes or less, 3 minutes or less, 2.5 minutes or less, 2 minutes or less, 1.5 minutes or less, or 1 minute or less. In some embodiments, the thermal denaturation incubation time is about any of 1 minute, 1.5 minutes, 2 minutes, 2.5 minutes, 3 minutes, 3.5 minutes, 4 minutes, 4.5 minutes, 5 minutes, 5.5 minutes, 6 minutes, 6.5 minutes, 7 minutes, 7.5 minutes, 8 minutes, 8.5 minutes, 9 minutes, 9.5 minutes, 10 minutes, 11 minutes, 12 minutes, 13 minutes, 14 minutes, or 15 minutes.

In some embodiments, the thermal denaturation technique comprises a thermal denaturation incubation time of about 1 minute to about 15 minutes, such as about any of 1 minute, 2 minutes, 3 minutes, 4 minutes, 5 minutes, 6 minutes, 7 minutes, 8 minutes, 9 minutes 10 minutes, 11 minutes, 12 minutes, 13 minutes, 14 minutes, or 15 minutes, wherein the thermal denaturation incubation is performed at a temperature of about 60° C. to about 100° C., such about any of 60° C., 65° C., 70° C., 75° C., 80° C., 85° C., 90° C., 91° C., 92° C., 93° C., 94° C., 95° C., 96° C., 97° C., 98° C., 99° C., or 100° C.

The thermal denaturation incubation temperature can be controlled by numerous techniques and combinations thereof. In some embodiments, the thermal denaturation incubation temperature is controlled by a water bath. In some embodiments, the thermal denaturation incubation temperature is controlled by a heat block. In some embodiments, the thermal denaturation incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the thermal denaturation incubation time temperature are controlled by a technique described herein, such as cooling at room temperature or via the thermocycler.

In some embodiments, the thermal denaturation technique comprises subjecting a sample, or a derivative thereof, e.g., a sample diluted in a buffer, to a thermal cycle. In some embodiments, the thermal cycle comprises subjecting the sample, or a derivative thereof, to one or more of: (a) a block starting temperature (b) block set temperature (the temperature for the thermal denaturation incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 60° C. to about 100° C., including about any of 60° C., 65° C., 70° C., 75° C., 80° C., 85° C., 90° C., 91° C., 92° C., 93° C., 94° C., 95° C., 96° C., 97° C., 98° C., 99° C., or 100° C. In some embodiments, the thermal cycle comprises: (a) a set block temperature of about 60° C. to about 100° C., including about any of 60° C., 65° C., 70° C., 75° C., 80° C., 85° C., 90° C., 91° C., 92° C., 93° C., 94° C., 95° C., 96° C., 97° C., 98° C., 99° C., or 100° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 50° C., such as any of 15° C. to about 25° C., about 20° C. to about 30° C., or about 20° C. to about 25° C., including about any of 20° C., 21° C., 22° C., 23° C., 24° C., 25° C., (b) a set block temperature of about 60° C. to about 100° C., such about any of 60° C., 65° C., 70° C., 75° C., 80° C., 85° C., 90° C., 91° C., 92° C., 93° C., 94° C., 95° C., 96° C., 97° C., 98° C., 99° C., or 100° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C. In some embodiments, the lid temperature during the thermal cycle may be the same respective temperature of the block during the thermal cycle or a temperature greater than the temperature of the block during the thermal cycle. In some embodiments, the lid temperature during the thermal cycle may be the same respective temperature of the block during the thermal cycle or a temperature greater than the temperature of the block during the thermal cycle.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

In some embodiments, the method further comprises admixing an amount of a blood sample, or portion thereof, a buffer prior to the thermal denaturation technique (e.g., the buffered sample is subjected to a thermal denaturation technique described herein). In some embodiment, the amount (as assessed based on the final concentration in the sample containing solution containing solution) of the buffer is about 1 mM to about 100 mM, such as any of about 20 mM to about 80 mM, about 30 mM to about 70 mM, or about 40 mM to about 60 mM. In some embodiment, the amount of the buffer is about any of 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some embodiments, the buffer is selected from the group consisting of ammonium bicarbonate, ammonium acetate, ammonium formate, triethylammonium bicarbonate, and Tris-HCl, or any combination thereof.

In some embodiments, the method further comprises determining the protein concentration in a blood sample or a derivative thereof.

### II. Reduction Techniques

In certain aspects, the methods provided herein comprise performing a reduction technique. In some embodiments, the reduction technique is performed on a sample, or a derivative thereof, following thermal denaturation. Reduction techniques, generally speaking, reduce (e.g., cleave) disulfide linkages between cysteine residues of one or more polypeptides to reduce the presence of polypeptide conformations that inhibit or prevent protease cleavage of the one or more polypeptides. Reduction techniques described herein comprise subjecting a sample, or a derivative thereof (e.g., a denatured sample), to an amount of a reducing agent and incubating for a reducing incubation time performed at a temperature or range thereof.

In some embodiments, the reducing agent is dithiothreitol (DTT), tris(2-carboxyethyl) phosphine (TCEP), beta-mercaptoethanol (BME), or a cysteine, or any mixture thereof.

In some embodiments, the amount (as assessed based on the final concentration in the sample containing solution containing solution) of a reducing agent, e.g., DTT, used in a reduction technique is about 1 mM to about 100 mM, such as any of about 1 mM to about 40 mM, about 1 mM to about 30 mM, about 5 mM to about 25 mM, about 10 mM, to about 20 mM, 20 mM to about 80 mM, about 30 mM to about 70 mM, or about 40 mM to about 60 mM. In some embodiments, the amount of reducing agent used in a reduction technique is at least about 1 mM, such as at least about any of 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some embodiments, the amount of reducing agent used in a reduction technique is about 100 mM or less, such as about any of 95 mM or less, 90 mM or less, 85 mM or less, 80 mM or less, 75 mM or less, 70 mM or less, 65 mM or less, 60 mM or less, 55 mM or less, 50 mM or less, 45 mM or less, 40 mM or less, 35 mM or less, 30 mM or less, 25 mM or less, 20 mM or less, 19 or less, 18 or less, 17 or less, 16 or less, 15 or less, 14 or less, 13 or less, 12 or less, 11 or less, 10 or less, 9 or less, 8 or less, 7 or less, 6 or less, 5 or less, 4 or less, 3 or less, 2 or less, or 1 or less. In some embodiments, the amount of reducing agent used in a reduction technique is about any of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In some embodiments, the reduction incubation time is about 10 minutes to about 120 minutes, such as any of about 30 minutes to about 60 minutes, about 40 minutes to about 60 minutes, about 45 minutes to about 55 minutes. In some embodiments, the reduction incubation time is at least about 20 minutes, such as at least about any of 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes. In some embodiments, the reduction incubation time is about 120 minutes or less, such as about any of 115 minutes or less, 110 minutes or less, 105 minutes or less, 100 minutes or less, 95 minutes or less, 90 minutes or less, 85 minutes or less, 80 minutes or less, 75 minutes or less, 70 minutes or less, 65 minutes or less, 60 minutes or less, 55 minutes or less, 50 minutes or less, 45 minutes or less, 40 minutes or less, 35 minutes or less, 30 minutes or less, or 25 minutes or less. In some embodiments, the reduction incubation time is about any of 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes.

In some embodiments, the reduction incubation time is performed at a temperature of about 20° C. to about 100° C., such as any of about 40° C. to about 80° C., about 50° C. to about 70° C., about 50° C. to about 60° C., about 55° C. to about 65° C., or about 60° C. to about 70° C. In some embodiments, the reduction incubation time is performed at a temperature of at least about 20° C., such as at least about any of 25° C., 30° C., 35° C., 40° C., 45° C., 50° C., 55° C., 60° C., 65° C., 70° C., 75° C., 80° C., 85° C., 90° C., or 95° C. In some embodiments, the reduction incubation time is performed at a temperature of about 95° C. or less, such as about any of 90° C. or less, 85° C. or less, 80° C. or less, 75° C. or less, 70° C. or less, 65° C. or less, 60° C. or less, 55° C. or less, 50° C. or less, 45° C. or less, 40° C. or less, 35° C. or less, 30° C. or less, or 25° C. or less. In some embodiments, the reduction incubation time is performed at a temperature of about any of 20° C., 25° C., 30° C., 35° C., 40° C., 45° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 75° C., 80° C., 85° C., 90° C., or 95° C. In some embodiments, the reduction incubation time is performed at a room temperature.

In some embodiments, the reduction technique comprises a reduction incubation time of about 30 minutes to about 70 minutes, such as about any of 35 minutes, 40 minutes, 45 minutes, 50 minutes, 55 minutes, 60 minutes, or 65 minutes, wherein the reduction incubation time is performed at a temperature of about 50° C. to about 70° C., such about any of 55° C., 60° C., or 65° C. In some embodiments, the reduction technique comprises use of an amount (as assessed based on the final concentration in the sample containing solution) of a reducing agent, e.g., DTT, of about 5 mM to about 25 mM, such as any of about 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, or 24 mM, and a reduction incubation time of about 30 minutes to about 70 minutes, such as about any of 35 minutes, 40 minutes, 45 minutes, 50 minutes, 55 minutes, 60 minutes, or 65 minutes, wherein the reduction incubation time is performed at a temperature of about 50° C. to about 70° C., such about any of 55° C., 60° C., or 65° C.

The reduction incubation temperature can be controlled by numerous techniques and combinations thereof. In some embodiments, the reduction incubation temperature is controlled by an ambient temperature, such as room temperature. In some embodiments, the reduction incubation temperature is controlled by a water bath. In some embodiments, the reduction incubation temperature is controlled by a heat block. In some embodiments, the reduction incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the reduction incubation time temperature are controlled by a technique described herein, such as cooling at room temperature or a ramp rate.

In some embodiments, the reduction technique comprises subjecting a sample, or a derivative thereof, e.g., a denatured sample, to a thermal cycle. In some embodiments, the thermal cycle comprises subjecting the sample, or a derivative thereof, to one or more of: (a) a block starting temperature (b) block set temperature (the temperature for the reduction incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 20° C. to about 100° C., such as any of 40° C. to about 80° C., about 50° C. to about 70° C., about 50° C. to about 60° C., about 55° C. to about 65° C., or about 60° C. to about 70° C., including about any of 50° C., 55° C., 60° C., 65° C., or 70° C. In some embodiments, the thermal cycle comprises: (a) a set block temperature of about 20° C. to about 100° C., such as any of about 40° C. to about 80° C., about 50° C. to about 70° C., about 50° C. to about 60° C., about 55° C. to about 65° C., or about 60° C. to about 70° C., including about any of 50° C., 55° C., 60° C., 65° C., or 70° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 60° C., such as any of about 15° C. to about 50° C., about 20° C. to about 40° C., about 20° C. to about 30° C., or about 20° C. to about 25° C., (b) a set block temperature of about 20° C. to about 100° C., such as any of about 40° C. to about 80° C., about 50° C. to about 70° C., about 50° C. to about 60° C., about 55° C. to about 65° C., or about 60° C. to about 70° C., including about any of 50° C., 55° C., 60° C., 65° C., or 70° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

In some embodiments, the reduction technique described herein is completed simultaneously with a thermal denaturation step. For example, the combined thermal denaturation technique and reduction technique comprises adding a reducing agent to a sample, or a derivative thereof, and then subjecting the sample, or the derivative thereof, to a temperature of about 60° C. to about 100° C., such about any of 60° C., 65° C., 70° C., 75° C., 80° C., 85° C., 90° C., 91° C., 92° C., 93° C., 94° C., 95° C., 96° C., 97° C., 98° C., 99° C., or 100° C., for an incubation time of at least about 1 minute, such as at least about any of 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes, or 60 minutes. In some embodiments, the combined thermal denaturation technique and reduction technique comprises adding a reducing agent to a sample, or a derivative thereof, and then subjecting the sample, or the derivative thereof, to a temperature of about 90° C. to about 100° C., for an incubation time of about 40 minutes to about 60 minutes, including 50 minutes.

### III. Alkylation Techniques

In certain aspects, the methods provided herein comprise performing an alkylation technique. In some embodiments, the alkylation technique is performed on a sample, or a derivative thereof, following the performance of a reduction technique. Alkylation techniques, generally speaking, prevent the reformation of one or more disulfide linkages between, e.g., cysteine residues of one or more polypeptides. This is done by, e.g., the addition of an acetamide moiety to the sulfur of a cysteine residue thereby producing an alkylated polypeptide. Alkylation techniques may reduce the presence of polypeptide conformations that inhibit or prevent protease cleavage of the one or more polypeptides. Alkylation techniques described herein comprise subjecting a sample, or a derivative thereof (e.g., a reduced sample), to an amount of an alkylating agent and incubating for an alkylation incubation time performed at a temperature or range thereof. In some embodiments, the method comprises subjecting a denatured sample to a reduction technique followed by an alkylation technique prior to performing a proteolytic digestion technique.

In some embodiments, the alkylating agent is iodoacetamide (IAA), 2-chloroacetamide, an acetamide salt, or any mixture thereof.

In some embodiments, the amount (as assessed based on the final concentration in the sample containing solution) of an alkylating agent, e.g., IAA, used in an alkylation technique is about 10 mM to about 100 mM, such as any of about 10 mM to about 50 mM, about 20 mM to about 40 mM, about 20 mM to about 36 mM, about 15 mM to about 25 mM, about 20 mM to about 25 mM, about 20 mM to about 80 mM, about 30 mM to about 70 mM, or about 40 mM to about 60 mM. In some embodiments, the amount of an alkylating agent used in an alkylation technique is at least about 10 mM, such as at least about any of 15 mM, 16 mM, 17 mM, 18 mM, 19 mM, 20 mM, 21 mM, 22 mM, 23 mM, 24 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some embodiments, the amount of an alkylating agent used in an alkylation technique is about 100 mM or less, such as about any of 95 mM or less, 90 mM or less, 85 mM or less, 80 mM or less, 75 mM or less, 70 mM or less, 65 mM or less, 60 mM or less, 55 mM or less, 50 mM or less, 45 mM or less, 40 mM or less, 35 mM or less, 30 mM or less, 25 mM or less, 24 mM or less, 23 mM or less, 22 mM or less, 21 mM or less, 20 mM or less, 19 mM or less, 18 mM or less, 17 mM or less, 16 mM or less, 15 mM or less, or 10 mM or less. In some embodiments, the amount of an alkylating agent used in an alkylation technique is about any of 10 mM, 15 mM, 20 mM, 20.5 mM, 21 mM, 21.5 mM, 22 mM, 22.5 mM, 23 mM, 23.5 mM, 24 mM, 24.5 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In some embodiments, the alkylation incubation time is about 5 minutes to about 60 minutes, such as any of about 10 minutes to about 50 minutes, about 20 minutes to about 40 minutes, about 25 minutes to about 35 minutes. In some embodiments, the alkylation incubation time is at least about 5 minutes, such as at least about any of 10 minutes, 15 minutes, 20 minutes, 25 minutes, 30 minutes, 35 minutes, 40 minutes, 45 minutes, 50 minutes, 55 minutes, or 60 minutes. In some embodiments, the alkylation incubation time is about 60 minutes or less, such as about any of 55 minutes or less, 50 minutes or less, 45 minutes or less, 40 minutes or less, 35 minutes or less, 30 minutes or less, 25 minutes or less, 20 minutes or less, 15 minutes or less, 10 minutes or less, or 5 minutes or less. In some embodiments, the alkylation incubation time is about any of 5 minutes, 10 minutes, 15 minutes, 20 minutes, 25 minutes, 30 minutes, 35 minutes, 40 minutes, 45 minutes, 50 minutes, 55 minutes, or 60 minutes.

In some embodiments, the alkylation incubation time is performed at a temperature of about 15° C. to about 100° C., such as any of about 15° C. to about 80° C., about 15° C. to about 60° C., about 15° C. to about 35° C., about 20° C. to about 30° C., or about 20° C. to about 25° C. In some embodiments, the alkylation incubation time is performed at a temperature of at least about 15° C., such as at least about any of 20° C., 21° C., 22° C., 23° C., 24° C., 25° C., 30° C., 35° C., 40° C., 45° C., 50° C., 55° C., 60° C., 65° C., 70° C., 75° C., 80° C., 85° C., 90° C., or 95° C. In some embodiments, the alkylation incubation time is performed at a temperature of about 95° C. or less, such as about any of 90° C. or less, 85° C. or less, 80° C. or less, 75° C. or less, 70° C. or less, 65° C. or less, 60° C. or less, 55° C. or less, 50° C. or less, 45° C. or less, 40° C. or less, 35° C. or less, 30° C. or less, 25° C. or less, 24° C. or less, 23° C. or less, 22° C. or less, 21° C. or less, or 20° C. or less. In some embodiments, the alkylation incubation time is performed at a temperature of about any of 15° C., 16° C., 17° C., 18° C., 19° C., 20° C., 21° C., 22° C., 23° C., 24° C., 25° C., 30° C., 35° C., 40° C., 45° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 75° C., 80° C., 85° C., 90° C., or 95° C. In some embodiments, the alkylation incubation time is performed at a room temperature.

In some embodiments, the alkylation technique comprises an alkylation incubation time of about 5 minutes to about 60 minutes, such as about any of 15 minutes, 20 minutes, 25 minutes, 30 minutes, 35 minutes, or 40 minutes, wherein the alkylation incubation time is performed at a temperature of about 15° C. to about 30° C., such about any of 20° C., 21° C., 22° C., 23° C., 24° C., 25° C. In some embodiments, the alkylation technique comprises use of an amount (containing solution based on the final concentration in the sample) of an alkylating agent, e.g., IAA, of about 15 mM to about 40 mM, such as any of about 20 mM, 20.5 mM, 21 mM, 21.5 mM, 22 mM, 22.5 mM, 23 mM, 23.5 mM, 24 mM, 24.5 mM, 25 mM, 25.5 mM, 26 mM, 26.5 mM, 27 mM, 27.5 mM, 28 mM, 28.5 mM, 29 mM, 29.5 mM, 30 mM, 30.5 mM, 31 mM, 31.5 mM, 32 mM, 32.5 mM, 33 mM, 33.5 mM, 34 mM, 34.5 mM, 35 mM, 35.5 mM, 36 mM, 36.5 mM, 37 mM, 37.5 mM, 38 mM, and an alkylation incubation time of about 5 minutes to about 60 minutes, such as about any of 15 minutes, 20 minutes, 25 minutes, 30 minutes, 35 minutes, or 40 minutes, wherein the alkylation incubation time is performed at a temperature of about 15° C. to about 30° C., such about any of 20° C., 21° C., 22° C., 23° C., 24° C., or 25° C.

The alkylation incubation temperature can be controlled by numerous techniques and combinations thereof. In some embodiments, the alkylation incubation temperature is controlled by an ambient temperature, such as room temperature. In some embodiments, the alkylation incubation temperature is controlled by a water bath. In some embodiments, the alkylation incubation temperature is controlled by a heat block. In some embodiments, the alkylation incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the alkylation incubation time temperature are controlled by a technique described herein, such as cooling at room temperature or a ramp rate.

In some embodiments, the alkylation technique comprises subjecting a sample, or a derivative thereof, e.g., a denatured sample, to a thermal cycle. In some embodiments, the thermal cycle comprises subjecting the sample, or a derivative thereof, to one or more of: (a) a block starting temperature (b) block set temperature (the temperature for the alkylation incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., or 25° C. In some embodiments, the thermal cycle comprises: (a) a set block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., 25° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., 25° C., (b) a set block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., 25° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C.

In some embodiments, the alkylation technique further comprises quenching the alkylating agent comprising use of a neutralizing agent. In some embodiments, the neutralizing agent is a reducing agent. In some embodiments, the reducing agent is dithiothreitol (DTT), tris(2-carboxyethyl) phosphine (TCEP), beta-mercaptoethanol (BME), or a cysteine, or any mixture thereof. In some embodiments, the neutralizing agent is added in an amount to fully quench the amount of the alkylating agent, such as in an amount greater than or equal to a molar amount of an active moiety of the alkylating agent. In some embodiments, the amount (as assessed based on the final concentration in the sample containing solution) of the neutralizing agent is about 1 mM to about 100 mM.

In some embodiments, the alkylation technique, in whole or in part, is performed substantially in a low light condition. In some embodiments, the alkylation incubation time is performed in a low light condition. In some embodiments, the low light condition is in the dark or a location substantially devoid of sunlight and/or room lighting, such as in a desk drawer. In some embodiments, the low light condition is a filtered light, such as red light.

In some embodiments, the alkylating agent is sourced from a stock solution. In some embodiments, the stock solution is prepared within about 1 hour, such as within about any of 50 minutes, 40 minutes, 30 minutes, 20 minutes, or 10 minutes, of use.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

### IV. Proteolytic Digestion Techniques

In certain aspects, the methods provided herein comprise performing a proteolytic digestion technique. In some embodiments, the proteolytic digestion technique is performed on a sample, or a derivative thereof, following thermal denaturation and/or any additional steps intended to expose protease cleavage sites. Proteolytic digestion techniques, generally speaking, cleave polypeptides at known cleavage sites. For example, trypsin is a serine protease that generally cleaves polypeptides at the carboxyl side (C-terminal side) of lysine and arginine residues. Certain exceptions apply to the cleavage pattern of trypsin, such as due to proximity of a proline residue and/or a post-translational modification causing steric hindrance relative to the cleavage site. Proteolytic digestion techniques described herein comprise subjecting a sample, or a derivative thereof (e.g., a denatured sample or an alkylated sample, including an alkylated sample subjected to a reduction technique prior to an alkylation technique), to an amount of one or more proteases and incubating for a digestion incubation time performed at a temperature or range thereof.

In some embodiments, each of the one or more proteases is trypsin, LysC, LysN, AspN, GluC, ArgC, IdeS, IdeZ, PNGase F, thermolysin, pepsin, elastase, TEV, or Factor Xa, or any mixture thereof. In some embodiments, wherein two or more proteases are used, the weight ratio between a first protease and a second protease is about 1:10 to about 10:1, such as about any of about 1:9, 1:8, 1:7: 1:6, 1:5, 1:4, 1:3, 1:2, or 1:1. In some embodiments, the one or more proteases is trypsin. In some embodiments, the one or more proteases is a mixture of trypsin and LysC, such as in a weight ratio of about 1:1. In some embodiments, the one or more proteases is selected based on the type and/or characteristic of a blood sample, or components thereof, used in the methods herein. In some embodiments, the blood sample is processed by the absorbent or bibulous member such that a plasma sample is obtained, wherein the one or more proteases is trypsin and Lys-C, such as in a weight ratio of about 1:1. In some embodiments, the blood sample is processed by the absorbent or bibulous member such that a serum sample is obtained, wherein the one or more proteases is trypsin. In some embodiments, the protease is a modified protease, such as comprising a modification to prevent or inhibit self-proteolysis. In some embodiments, the modified protease is a modified trypsin, such as a methylated and/or an acetylated trypsin. In some embodiments, the modified trypsin is a tosyl phenylalanyl chloromethyl ketone (TPCK)-treated trypsin.

In some embodiments, the amount of a protease, e.g., trypsin or LysC, used in a proteolytic digestion technique is based on a weight ratio relative to the polypeptide content of a sample, or a derivative thereof, (i.e., weight of a protease: weight of polypeptide content) of about 1:200 to about 1:10, such as any of about 1:100 to about 1:10, about 1:50 to about 1:10, about 1:40 to about 1:20, about 1:50 to about 1:30, about 1:45 to about 1:35, about 1:20 to about 1:40, about 1:30 to about 1:10, or about 1:25 to about 1:15. In some embodiments, the amount of a protease used in a proteolytic digestion technique is at least about 1:200, such as at least about any of 1:190, 1:180, 1:170, 1:160, 1:150, 1:140, 1:130, 1:120, 1:110, 1:100, 1:95, 1:90, 1:85, 1:80, 1:75, 1:70, 1:65, 1:60, 1:55, 1:50, 1:45, 1:40, 1:35, 1:30, 1:25, 1:20, 1:15, or 1:10. In some embodiments, the amount of a protease used in a proteolytic digestion technique is about 1:10 or less, such as about any of 1:15 or less, 1:20 or less, 1:25 or less, 1:30 or less, 1:35 or less, 1:40 or less, 1:45 or less, 1:50 or less, 1:55 or less, 1:60 or less, 1:65 or less, 1:70 or less, 1:75 or less, 1:80 or less, 1:85 or less, 1:90 or less, 1:95 or less, 1:100 or less, 1:110 or less, 1:120 or less, 1:130 or less, 1:140 or less, 1:150 or less, 1:160 or less, 1:170 or less, 1:180 or less, 1:190 or less, or 1:200 or less. In some embodiments, the amount of a protease used in a proteolytic digestion technique is about any of 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, 1:110, 1:120, 1:130, 1:140, 1:150, 1:160, 1:170, 1:180, 1:190, or 1:200. In some embodiments, the proteolytic digestion technique comprises the use of two or more proteases, such as a combination of trypsin and LysC, and in such embodiments, the amount of each protease (such as described above) can be summed to a total amount of proteases used in a proteolytic digestion technique.

In some embodiments, the proteolytic digestion incubation time is about 20 minutes to about 36 hours, such as any of about 1 hour to about 18 hours, about 5 hours to about 24 hours, about 12 hours to about 24 hours, about 16 hours to about 20 hours, or about 12 hours to about 36 hours. In some embodiments, the proteolytic digestion incubation time is about 36 hours or less, such as about any of 32 hours or less, 30 hours or less, 28 hours or less, 26 hours or less, 24 hours or less, 22 hours or less, 20 hours or less, 19 hours or less, 18 hours or less, 17 hours or less, 16 hours or less, 15 hours or less, 14 hours or less, 13 hours or less, 12 hours or less, 11 hours or less, 10 hours or less, 9 hours or less, 8 hours or less, 7 hours or less, 6 hours or less, 5 hours or less, 4 hours or less, 3 hours or less, 2 hours or less, or 1 hours or less. In some embodiments, the proteolytic digestion incubation time is at least about 20 minutes, such as at least about any of 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 22 hours, 24 hours, 26 hours, 28 hours, 30 hours, 32 hours, 34 hours, or 36 hours. In some embodiments, the proteolytic digestion incubation time is about any of 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 22 hours, 24 hours, 26 hours, 28 hours, 30 hours, 32 hours, 34 hours, or 36 hours.

In some embodiments, the digestion incubation time is performed at a temperature of about 20° C. to about 60° C., such as any of about 20° C. to about 25° C., about 20° C. to about 30° C., about 25° C. to about 40° C., about 35° C. to about 40° C., or about 35° C. to about 50° C. In some embodiments, the digestion incubation time is performed at a temperature of at least about 20° C., such as at least about any of 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 42° C., 44° C., 46° C., 48° C., 50° C., 52° C., 54° C., 56° C., 58° C., or 60° C. In some embodiments, the digestion incubation time is performed at a temperature of about 60° C. or less, such about any of 58° C. or less, 56° C. or less, 54° C. or less, 52° C. or less, 50° C. or less, 48° C. or less, 46° C. or less, 44° C. or less, 42° C. or less, 40° C. or less, 39° C. or less, 38° C. or less, 37° C. or less, 36° C. or less, 35° C. or less, 34° C. or less, 33° C. or less, 32° C. or less, 31° C. or less, 30° C. or less, 29° C. or less, 28° C. or less, 27° C. or less, 26° C. or less, 25° C. or less, 24° C. or less, 23° C. or less, 22° C. or less, 21° C. or less, or 20° C. or less. In some embodiments, the digestion incubation time is performed at a temperature of about any of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 42° C., 44° C., 46° C., 48° C., 50° C., 52° C., 54° C., 56° C., 58° C., or 60° C. In some embodiments, the reduction incubation time is performed at a room temperature.

In some embodiments, the proteolytic digestion technique comprises a digestion incubation time of about 12 hours to about 24 hours, such as about any of 13 hours, 14 hours, 15 hours, 16 hours, 17 hours, 18 hours, 19 hours, 20 hours, 21 hours, 22 hours, 23 hours, wherein the digestion incubation time is performed at a temperature of about 20° C. to about 40° C., such about any of 22° C., 24° C., 26° C., 28° C., 30° C., 31° C., 32° C., 33° C., 34° C., 35° C., 36° C., 37° C., 38° C., 39° C. In some embodiments, the proteolytic digestion technique comprises use of an amount a protease for each of one or more proteases, e.g., trypsin and/or LysC, of about 1:15 to about 1:45, such as about any of 1:20, 1:25, 1:30, 1:35, or 1:40 (as measured based on the amount of the protease to the amount of polypeptide in a sample or a derivative thereof), and a digestion incubation time of about 12 hours to about 24 hours, such as about any of 13 hours, 14 hours, 15 hours, 16 hours, 17 hours, 18 hours, 19 hours, 20 hours, 21 hours, 22 hours, 23 hours, wherein the digestion incubation time is performed at a temperature of about 20° C. to about 40° C., such about any of 22° C., 24° C., 26° C., 28° C., 30° C., 31° C., 32° C., 33° C., 34° C., 35° C., 36° C., 37° C., 38° C., 39° C. In some embodiments, the proteolytic digestion technique is performed on a plasma sample or a derivate thereof, wherein the proteolytic digestion technique comprises a 1:40 ratio of trypsin to polypeptide in the sample of the derivative thereof and a 1:40 ratio of LysC to polypeptide in the sample or the derivative thereof. In some embodiments, the proteolytic digestion technique is performed on a serum sample or a derivate thereof, wherein the proteolytic digestion technique comprises a 1:20 ratio of trypsin.

The digestion incubation temperature can be controlled by numerous techniques and combinations thereof. In some embodiments, the digestion incubation temperature is controlled by an ambient temperature, such as room temperature. In some embodiments, the digestion incubation temperature is controlled by a water bath. In some embodiments, the digestion incubation temperature is controlled by a heat block. In some embodiments, the digestion incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the digestion incubation time temperature are controlled by a technique described herein, such as cooling at room temperature or a ramp rate.

In some embodiments, the proteolytic digestion technique comprises subjecting a sample, or a derivative thereof, e.g., an alkylated sample (including an alkylated sample quenched with a neutralizing agent), to a thermal cycle. In some embodiments, the thermal cycle comprises subjecting the sample, or a derivative thereof, to one or more of: (a) a block starting temperature (b) block set temperature (the temperature for the digestion incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 20° C. to about 50° C., including about any of 22° C., 24° C., 26° C., 28° C., 30° C., 31° C., 32° C., 33° C., 34° C., 35° C., 36° C., 37° C., 38° C., 39° C., 40° C., 42° C., 44° C., 46° C., 48° C.

In some embodiments, the thermal cycle comprises: (a) a set block temperature of about 20° C. to about 50° C., including about any of 22° C., 24° C., 26° C., 28° C., 30° C., 31° C., 32° C., 33° C., 34° C., 35° C., 36° C., 37° C., 38° C., 39° C., 40° C., 42° C., 44° C., 46° C., 48° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 35° C., such as any of about 20° C. to about 35° C., or about 20° C. to about 25° C., (b) a set block temperature of about 20° C. to about 50° C., including about any of 22° C., 24° C., 26° C., 28° C., 30° C., 31° C., 32° C., 33° C., 34° C., 35° C., 36° C., 37° C., 38° C., 39° C., 40° C., 42° C., 44° C., 46° C., 48° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

In some embodiments, the proteolytic digestion technique further comprises quenching the one or more proteolytic enzymes. In some embodiments, quenching the one or more proteolytic enzymes comprises denaturing the one or more proteolytic enzymes. In some embodiments, quenching the one or more proteolytic enzymes comprise adding an amount of an acid. In some embodiments, the acid is formic acid (FA) or trifluoroacetic acid (TFA), or a mixture thereof. In some embodiments, the amount (as assessed based on the final concentration in the sample containing solution) of the acid added is about any of 0.1% v/v, 0.2% v/v, 0.3% v/v, 0.4% v/v, 0.5% v/v, 0.6% v/v, 0.7% v/v, 0.8% v/v, 0.9% v/v, 1% v/v, 1.1% v/v, 1.2% v/v, 1.3% v/v, 1.4% v/v, 1.5% v/v, 1.6% v/v, 1.7% v/v, 1.8% v/v, 1.9% v/v, or 2% v/v.

### V. Additional Techniques

In certain aspects, the method provided herein comprise subjecting the proteolytically digested sample comprising a proteolytic glycopeptide to one or more additional steps prior to subjecting the proteolytically digested sample, or a derivative thereof, to a liquid chromatography-mass spectrometry (LC-MS) technique using a liquid chromatography system and a mass spectrometer. In some embodiments, the LC system is online with the MS (i.e., eluate from the LC system is directly introduced to the MS). In some embodiments, the one or more additional steps do not include a desalting step performed outside of the LC system (such as an offline desalting technique).

In some embodiments, the method further comprises adding a standard to the proteolytically digested sample prior to the LC-MS technique. In some embodiments, the standard is a stable isotope-internal standard (SI-IS) peptide mixture.

In some embodiments, the biological sample is not subjected to a high-abundant protein depletion technique prior to the thermal denaturation technique. For example, in some embodiments, the high-abundant protein depletion technique removes highly abundant proteins present in a blood sample, such as serum albumin.

### E3. Methods for Performing a LC-MS Analysis of a Proteolytic Glycopeptide

In certain aspects, provided herein is a method for performing a LC-MS analysis on a sample comprising a proteolytic glycopeptide.

In some embodiments, the liquid chromatography (LC) system is online with a mass spectrometer (i.e., proteolytic peptide species, including glycopeptides, are eluted from the LC system directing into the mass spectrometer via a mass spectrometer interface. In some embodiments, the LC technique comprises performing a chromatographic separation of one or more proteolytic peptides, including glycopeptides. In some embodiments, the one or more proteolytic peptides subjected to a chromatographic separation are obtained from a proteolytically digested sample, such as described herein. In some embodiments, the chromatographic separation is performed on a proteolytically digested sample, such as described herein, (e.g., no additional separation technique, such as a sample clean-up step, is performed to remove one or more components from proteolytically digested sample). In some embodiments, the chromatographic separation is performed on a proteolytically digested sample comprising at least about 5 mM of a buffer, such as ammonium bicarbonate. In some embodiments, the chromatographic separation is performed on a proteolytically digested sample comprising an amount, such as at least about 1 mM, of a reducing agent or a byproduct thereof, such as a stable six-membered ring with an internal disulfide bond derived from DTT. In some embodiments, the chromatographic separation is performed on a proteolytically digested sample comprising an amount, such as at least about 1 mM, of an alkylating agent or a byproduct thereof, such as iodide (I-) derived from IAA.

In some embodiments, the method comprises introducing the proteolytically digested sample to a LC-MS system. In some embodiments, the method comprises performing a chromatographic separation of the proteolytically digested sample. In some embodiments, the chromatography separation comprises a period of diversion (i.e., diverted from the mass spectrometer interface, e.g., to a waste receptacle) of an initial eluate from the proteolytically digested sample. In some embodiments, the initial eluate (as assessed from the sample front) diverted from the mass spectrometer is about 1 column volume of the chromatographic column to about 5 column volumes of the chromatographic column, such as any of about 1 column volumes to about 4 column volumes, about 2 column volumes to about 5 column volumes, or about 3 column volumes to about 4 column volumes. In some embodiments, the initial eluate (as assessed from the sample front) diverted from the mass spectrometer is at least about 0.5 column volumes, such as at least about any of 1 column volume, 1.5 column volumes, 2 column volumes, 2.5 column volumes, 3 column volumes, 3.5 column volumes, 4 column volumes, 4.5 column volumes, or 5 column volumes. In some embodiments, the initial eluate (as assessed from the sample front) diverted from the mass spectrometer is about 5 column volumes or less, such as about any of 4.5 column volumes or less, 4 column volumes or less, 3.5 column volumes or less, 3 column volumes or less, 2.5 column volumes or less, 2 column volumes or less, 1.5 column volumes or less, 1 column volume or less, or 0.5 column volumes or less. In some embodiments, the initial eluate (as assessed from the sample front) diverted from the mass spectrometer is about any of 0.5 column volumes, 1 column volume, 1.5 column volumes, 2 column volumes, 2.5 column volumes, 3 column volumes, 3.5 column volumes, 4 column volumes, 4.5 column volumes, or 5 column volumes.

In some embodiments, the proteolytically digest sample is subjected to a solid phase extraction column comprising a reversed phase material prior to subjecting the polypeptide content therein to a LC-MS analysis In some embodiments, the reversed-phase medium comprises an alkyl-based moiety, such as an alkyl-based moiety comprising an octadecyl carbon functional group (C18) covalently bound to a silica solid phase. In some embodiments, the silica solid phase comprises a plurality of particles having an average largest cross-sectional distance (such as a diameter, e.g., as measured by dynamic light scattering) of about 20 μm. In some embodiments, each particle of the plurality of particles comprises an average pore size of about 150 Å, and wherein the pores are derivitized with the alkyl-based moiety. In some embodiments, the solid phase extraction column comprising the reversed-phase medium has a column volume of about 5 μL. In some embodiments, the solid phase extraction column is an AssayMap 5 μL C18 cartridge (catalog no. 5190-6532; Agilent Technologies). In some embodiments, the reversed-phase medium comprises an underivitized polystyrene divinylbenzene hydrophobic reversed-phase resin. In some embodiments, the polystyrene divinylbenzene hydrophobic reversed-phase resin comprises a plurality of particles having an average largest cross-sectional distance (such as a diameter, e.g., as measured by dynamic light scattering) of about 20 μm. In some embodiments, each particle of the plurality of particles comprises an average pore size of about 100 Å. In some embodiments, the solid phase extraction column comprising the reversed-phase medium has a column volume of about 5 μL. In some embodiments, the solid phase extraction column is an AssayMAP 5 μL Reversed Phase (RP-S) cartridge (catalog no. G5496-60033; Agilent Technologies).

In some embodiments, the column volume refers to the volume occupied by the reversed-phase media within the sample phase extraction column or cartridge. The reversed-phase media can be referred to as a bed that is compacted within a chromatography column to form a bed volume.

In some embodiments, the chromatographic separation comprises a gradient separation performing using mixtures of an aqueous mobile phase and an organic mobile phase. In some embodiments, the chromatographic separation comprises isocratic period, such as a period of at least about 90%, such as at least about any of 91%, 92%, 93%, 94%, 95%, 96%, 97%, 98%, 99%, or 100%, of an aqueous mobile phase to produce the initial eluate that is diverted from the mass spectrometer interface.

In some embodiments, the LC system comprises a reversed-phase chromatography column. In some embodiments, the reversed-phase column comprises an alkyl moiety, such as C18.

The present application contemplates a diverse array of additional features of LC-MS techniques for analyzing a sample comprising a glycopeptide using a mass spectrometer. In some embodiments, the liquid chromatography system comprises a high performance liquid chromatography system. In some embodiments, the liquid chromatography system comprises an ultra-high performance liquid chromatography system. In some embodiments, the liquid chromatography system comprises a high-flow liquid chromatography system. In some embodiments, the liquid chromatography system comprises a low-flow liquid chromatography system, such as a micro-flow liquid chromatography system or a nano-flow liquid chromatography system. In some embodiments, the liquid chromatography system is coupled, such as directly interfaced, with a mass spectrometer.

In some embodiment, the mass spectrometry technique comprises an ionization technique. Ionization techniques contemplated by the present application include techniques capable of charging polypeptides and peptide products, including glycopeptides. Thus, in some embodiments, the ionization technique is electrospray ionization. In some embodiments, the ionization technique is nano-electrospray ionization. In some embodiments, the ionization technique is atmospheric pressure chemical ionization. In some embodiments, the ionization technique is atmospheric pressure photoionization.

A diverse array of mass spectrometers are contemplated as compatible with the description, include high-resolution mass spectrometers and low-resolution mass spectrometers. In some embodiments, the mass spectrometer is a time-of-flight (TOF) mass spectrometer. In some embodiments, the mass spectrometer is a quadrupole time-of-flight (Q-TOF) mass spectrometer. In some embodiments, the mass spectrometer is a quadrupole ion trap time-of-flight (QIT-TOF) mass spectrometer. In some embodiments, the mass spectrometer is an ion trap. In some embodiments, the mass spectrometer is a single quadrupole. In some embodiments, the mass spectrometer is a triple quadrupole (QQQ). In some embodiments, the mass spectrometer is an orbitrap. In some embodiments, the mass spectrometer is a quadrupole orbitrap. In some embodiments, the mass spectrometer is a fourier transform ion cyclotron resonance (FT) mass spectrometer. In some embodiments, the mass spectrometer is a quadrupole fourier transform ion cyclotron resonance (Q-FT) mass spectrometer. In some embodiments, the mass spectrometry technique comprises positive ion mode. In some embodiments, the mass spectrometry technique comprises negative ion mode. In some embodiments, the mass spectrometry technique comprises an ion mobility mass spectrometry technique.

In some embodiments, the LC-MS technique comprises processing obtained signals MS from the mass spectrometer. In some embodiments, the LC-MS technique comprises peak detection. In some embodiments, the LC-MS technique comprises determining ionization intensity of an ionized peptide product. In some embodiments, the LC-MS technique comprises determining peak height of an ionized peptide product. In some embodiments, the LC-MS technique comprises determining peak area of an ionized peptide product. In some embodiments, the LC-MS technique comprises determining peak volume of an ionized peptide product. In some embodiments, the LC-MS technique comprises identifying an ionized peptide product by amino acid sequence. In some embodiments, the LC-MS technique comprises determining the site of a post-translational modification of an ionized peptide, such as the site of a glycosylation. In some embodiments, the LC-MS technique comprises determining the glycan structure, or a characteristic thereof, of an ionized peptide product. In some embodiments, the LC-MS technique comprises manually validating the ionized peptide product acid sequence assignments. In some embodiments, the LC-MS technique comprises a quantification technique.

### F3. Samples and Components Thereof

The methods provided herein are contemplated to be suitable for analyzing a diverse array of samples, such as any blood samples or derivatives thereof obtained using an absorbent or bibulous member, such as a blood spot card. In some embodiments, the sample is a blood sample, such as a whole blood sample. In some embodiments, the blood sample is processed, such as by the absorbent or bibulous member, e.g., a lateral flow dried blood collection device. In some embodiments, the blood sample is a plasma sample. In some embodiments, the blood sample is a serum sample.

The methods provided herein are particularly useful for the analysis of blood samples, or portion thereof, comprising a glycoprotein, such as to generate glycopeptide containing specimens for analysis with a mass spectrometer.

In some embodiments, the method comprises an upstream sample preparation technique, such as for obtaining plasma or serum from a blood sample, performed prior to methods for proteolytically digesting a sample. In some embodiments, the upstream sample preparation technique comprises a cell lysis step. In some embodiments, the upstream sample preparation technique comprises a filtration step. In some embodiments, the upstream sample preparation technique comprises a dilution step. In some embodiments, the upstream sample preparation technique comprises a protein concentration determination step.

In some embodiments, the sample is obtained from an individual. In some embodiments, the sample is obtained from a human individual.

### G3. Systems, Kits, and Compositions

In certain aspects, contemplated herein are systems, kits, and compositions useful for performing the methods described herein. In some embodiments, provided herein is a system, kit, and/or composition useful for performing a proteolytic digestion of a blood sample, or a portion thereof, comprising a glycoprotein as described herein. In some embodiments, provided herein is a system, kit, and/or composition useful for performing a LC-MS analysis of a proteolytic glycopeptide as described herein. In some embodiments, provided herein is a system, kit, and/or composition useful for performing a proteolytic digestion of a blood sample, or a portion thereof, comprising a glycoprotein followed by LC-MS analysis of the proteolytic glycopeptide produced therefrom.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### Section 4—Method of Diagnosing Pelvic Tumors

Provided herein are methods of diagnosing a pelvic tumor, such as ovarian cancer, based upon the presence, absence, or amount of one or more biomarkers. In some embodiments, the biomarker is a glycopeptide. In some embodiments, the method comprises performing mass spectrometry on a sample derived from blood. In some embodiments, the method comprises performing mass spectrometry (such as LC-MS) on a sample deposited on an absorbent or bibulous member (such as a blood spot card).

Provided herein, in certain aspects, is an absorbent or bibulous member, such as a blood spot card, comprising one or more extraction internal standards comprising at least one polypeptide standard deposited thereon prior to deposition of a blood sample. In other aspects, provided herein are one or more extraction internal standards comprising a polypeptide standard, including mixture of polypeptide standards, suitable for use in glycopolypeptide LC-MS analyses of samples obtained from an absorbent or bibulous member, such as a dried blood spot card. In other aspects, provided herein are methods for performing a liquid chromatography-mass spectrometry (LC-MS) analysis of a proteolytic glycopeptide derived from a blood sample deposited on an absorbent or bibulous member, such as a dried blood spot card, such as for the analysis of one or more biomarkers, e.g., biomarkers for the diagnosis of ovarian cancer. In other aspects, provided herein are one or more biomarkers for assessing ovarian cancer in an individual suitable for assessment of polypeptide content obtained from an absorbent or bibulous member, such as a blood spot card. The disclosure of the present application is based on the inventors' unique perspective and unexpected findings regarding the use of absorbent or bibulous members, such as blood spot cards, for the analysis of glycopolypeptides, such as proteolytic glycopeptides. As described herein, the inventors identified that the use of one or more extraction internal standards applied to an absorbent or bibulous member, such as a blood spot card, prior to deposition of a blood sample can facilitate analytical aspects of an LC-MS analysis of proteolytic peptides therefrom, such as one or more proteolytic glycopeptide. As described herein, the one or more extraction internal standards enable determination of any one or more of extraction efficiency, digestion efficiency, and a sample migration pattern such as to improve the analysis of polypeptide content, such as one or more proteolytic glycopeptides, extracted from an absorbent or bibulous member. Specifically, as described herein, it was unexpectedly found that the taught methods involving absorbent or bibulous members, such as blood spot cards, and one or more extraction internal standards enable performance of an ovarian cancer biomarker screen to accurately and reliably determine the presence of benign or non-benign ovarian cancer. Such results obtained using biomarkers useful for the techniques involving absorbent or bibulous member samples described herein were in agreement with a biomarkers for an established liquid blood-based assay. Furthermore, using the methods of performing a LC-MS analysis involving the absorbent or bibulous members described herein, the inventors identified one or more biomarkers useful for assessing ovarian cancer in an individual. In addition to the significant advantages provided by the subject matter described herein that enable LC-MS analysis of polypeptide content, such as one or more proteolytic glycopeptides, the subject matter provided herein benefits from the use of dried blood sample, e.g., reduced risk of live blood biohazards, inhibition of enzymatic activity (e.g., proteases and glycotransferases), ease of obtaining a blood sample from an individual (including the small amount of blood sample required), and ease of transportation of a blood sample from the site of obtaining to a site of analysis.

Thus, in some aspects, provided herein is a method for performing a liquid chromatography-mass spectrometry (LC-MS) analysis of a proteolytic glycopeptide derived from a blood sample deposited on a delimited zone of an absorbent or bibulous member, wherein the blood sample comprises a plurality of polypeptides comprising at least one glycoprotein, the method comprising extracting at least a portion of the plurality of polypeptides and one or more extraction internal standards from the absorbent or bibulous member to obtain an extracted sample, wherein the absorbent or bibulous member comprises the one or more extraction internal standards prior to deposition of the blood sample within the delimited zone, and wherein at least one of the one or more extraction internal standards comprises a polypeptide standard; subjecting the extracted sample or a derivative thereof to a proteolytic digestion technique to produce a proteolytically digested sample comprising the proteolytic glycopeptide; introducing at least a portion of the proteolytically digested sample to a liquid chromatography (LC) system of a LC-MS system; and performing the LC-MS analysis on at least the proteolytic glycopeptide and the one or more extraction internal standards.

In other aspects, provided herein is a method for performing a liquid chromatography-mass spectrometry (LC-MS) analysis of a proteolytic glycopeptide derived from a blood sample from an individual deposited on a delimited zone of an absorbent or bibulous member, the method comprising obtaining an absorbent or bibulous member comprising a blood sample from the individual deposited thereon, wherein the absorbent or bibulous member comprises one or more extraction internal standards deposited and dried prior to deposition of the blood sample on the absorbent or bibulous member, and wherein the absorbent or bibulous member comprising the blood sample contains at least a portion of the blood sample and the one or more extraction internal standards in an overlapping area of the absorbent or bibulous member; extracting at least a portion of the plurality of polypeptides and the one or more extraction internal standards from the absorbent or bibulous member to obtain an extracted sample; subjecting the extracted sample or a derivative thereof to a proteolytic digestion technique to produce a proteolytically digested sample comprising the proteolytic glycopeptide; introducing at least a portion of the proteolytically digested sample to a liquid chromatography (LC) system of a LC-MS system; and performing an LC-MS analysis to quantify one or more biomarkers of ovarian cancer and the one or more extraction internal standards, and wherein at least one of the one or more biomarkers is a glycopeptide.

In other aspects, provided herein is an absorbent or bibulous member, such as a blood spot card, comprising one or more extraction internal standard, wherein the one or more extraction internal standards comprises at least one polypeptide standard, and wherein the blood spot card does not comprise a blood sample deposited thereon. In some embodiments, the one or more extraction standards are deposited on the absorbent or bibulous member, such as a blood spot card, in a delimited zone, such as where a blood sample is deposited.

B4. Example Mass Spectrometry and Sample Preparation Workflow with DBS

For purposes of orientation and illustration of the description herein, provided in this section are example aspects of sample preparation and mass spectrometry workflows (FIGS. 1A-1C) for analyzing the composition of a peptide and/or glycopeptide using a mass spectrometer. Subsequent sections are provided with more details regarding certain inventive features related to methods for proteolytically digesting a biological sample, such as a blood sample, comprising a glycoprotein, methods of performing a LC-MS analysis of a proteolytic glycopeptide, and mass spectrometry workflows involving any combination of elements thereof.

FIG. 1A is a schematic of an example workflow 100 for a peptide structure analysis, including of glycopeptides. The workflow 100 may include various operations including, for example, sample collection 102 using an absorbent or bibulous member, such as a blood spot card, sample intake 104, sample preparation and mass spectrometry processing 106, and data analysis 108.

Sample collection 102 may include, for example, obtaining a biological sample, such as a blood sample, 112 from an individual 114. Relevant to the methodology described herein, a biological sample 112 may take the form of a blood sample obtained via one or more sampling methods, such as a finger prick produced using a lancet, and deposited on an absorbent or bibulous member, such as a blood spot card. In some embodiments, many aliquots of the biological sample 112 are obtained 118, such as via deposition of a blood sample from an individual onto a plurality of absorbent or bibulous members or a plurality of blood deposition sites on an absorbent or bibulous member, such as a blood spot card. In some embodiments, the absorbent or bibulous members performs a separation of the blood sample from the individual, such as to produce a serum sample, a plasma sample, and/or extraction of a blood cell (e.g., white blood cell (WBC), red blood cell (RBC) sample. In some embodiments, the biological sample, such as the blood sample, 112 may include nucleotides (e.g., ssDNA, dsDNA, RNA), organelles, amino acids, peptides, proteins, carbohydrates, glycoproteins, or any combination thereof.

Sample intake 104 may include one or more various operations such as, for example, aliquoting, labeling, registering, processing, storing, thawing, and/or other types of operations involved with preparing a sample for sample preparation and mass spectrometry processing. In some embodiments, sample intake 104 comprises separating one or portions of an absorbent or bibulous member, such as punching one or more chads out of a blood spot card with a hole punching device.

Sample preparation and mass spectrometry processing 106 may include, for example, one or more operations to form set of peptide structures 122, such as a proteolytic peptide and/or a proteolytic glycopeptide. In some embodiments, the sample preparation comprises extracting one or more polypeptides from an absorbent or bibulous member, such as a blood spot card, or a portion thereof with an extraction buffer. In some embodiments, the extracted polypeptide content is further processed, such as via subjecting to a precipitation technique. In some embodiments, the sample preparation includes subjecting a blood sample, or a portion thereof, to a proteolytic digestion. Mass spectrometry processing 124 may include, for example, liquid chromatography, introducing species from the sample, and/or derived therefrom, to a mass spectrometer, and data acquisition, such as using a multiple reaction monitoring (MRM) technique. MRM is a mass spectrometry method in which a precursor ion of a particular m/z value, including window thereof, (e.g., peptide analyte) is selected in the first quadrupole (Q1) and transmitted to the second quadrupole (Q2) for fragmentation. The resulting product ions are then transmitted to the third quadrupole (Q3), which detects only product ions with selected predefined m/z values. In some embodiments, the predefined m/z value, including window thereof, selected in the first quadrupole and a predefined m/z value, including window thereof, may be expressed as a MRM transition. Dynamic MRM (dMRM) is a variant of MRM. In dynamic MRM mode, MRM transition lists are scheduled throughout an LC/MS run based on the retention time window for each analyte. In this way, analytes are only monitored while they are eluting from the LC and therefore the MS scan time is not wasted by monitoring the analytes when they are not expected.

### C4. Absorbent or Bibulous Members and Methods of Use Thereof

Provided herein, in certain aspects, are absorbent or bibulous members, such as a blood spot card or a lateral flow blood sample device, comprising one or more extraction internal standards, wherein at least one of the one or more extraction internal standards is a polypeptide standard. The absorbent or bibulous member and extraction internal standards, including methods associated therewith, are described in more detail below. The modular discussion of these features of the application is not intended to limit the scope of the description provided herein, and one of ordinary skill in the art will readily appreciate that various features can be combined.

### I. Absorbent or Bibulous Members

The absorbent or bibulous members encompassed herein may take many forms and, in some embodiments, perform one or more blood sample processing functions. Generally speaking, the absorbent or bibulous members described herein serve as a platform to accept a blood sample, or a derivative thereof, from an individual, wherein the blood sample, or the derivative thereof, can dry on or within the absorbent or bibulous member. Subsequently, the absorbent or bibulous member comprising a blood sample can be processed, e.g., shipped to a laboratory or subjected to an extraction technique. The absorbent or bibulous members described herein enhance the stability of analytes at ambient or elevated environmental temperatures (such as by inhibiting enzymatic activity in whole blood) and simplify the transportation of sample by avoiding the need for a cold chain. Blood spot cards are well known in the art, e.g., see U.S. Pat. Nos. 3,838,012; 9,198,609; and 10,422,729, which are hereby incorporated herein by reference in their entirety.

In some embodiments, the absorbent or bibulous member is a blood spot card. In some embodiments, the blood spot card comprises a porous material or a mesh material on which a blood sample, or portion thereof, is deposited and/or received. In some embodiments, the blood spot card comprises a filter paper material, such as an absorbent filter paper. In some embodiments, the filter paper material comprises a cellulose-based paper. In some embodiments, the filter paper material does not bind polypeptide content in a manner such that said polypeptide content cannot be later extracted from the filter paper material. In some embodiments the filter paper material prevents or reduces sample hemolysis. In some embodiments, the filter paper material is Watman paper. In some embodiments, the blood spot card is a Whatman 903 Proteinsaver card.

In some embodiments, the absorbent or bibulous member is a blood spot card comprising one or more delimited zones configured to guide placement of a blood sample. Such delimited zones may be any shape or size, and have any placement on the blood spot card. In some embodiments, the delimited zone has a surface area of about 100 mm² to about 1,000 mm², such as about any of 150 mm², 200 mm², 250 mm², 300 mm², 350 mm², 400 mm², 450 mm², 500 mm², 600 mm², 700 mm², 800 mm², or 900 mm². In some embodiments, the delimited zone has a surface area of about 1,000 mm² or less, such as about any of 900 mm² or less, 800 mm² or less, 700 mm² or less, 600 mm² or less, 500 mm² or less, 450 mm² or less, 400 mm² or less, 350 mm² or less, 300 mm² or less, 250 mm² or less, 200 mm² or less, 150 mm² or less, or 100 mm² or less. In some embodiments, the delimited zone forms a circle. In some embodiments, the delimited zone is marked, such as visible to the naked eye, but a line or a portion thereof, such as a dashed line. In some embodiments, the blood spot card comprises one or more additional markings, such as identifying information.

FIG. 14A provides an example blood spot card 1400 having three delimited zones 1404, 1406, 1408. The blood spot card 1400 comprises a filter paper material 1402. The delimited zones 1404, 1406, 1408 are marked by dashed line circles and direct the placement of a blood sample. The blood spot card 1400 further comprises a marking 1410 for the identification of the sample.

In some embodiments, the blood spot card is a patterned blood spot card comprising a feature to control the area of the card exposed to a blood sample. In some embodiments, the patterned blood spot card comprises a hydrophobic material, such as a wax, to control the area of the card exposed to a blood sample.

In some embodiments, the blood spot card further comprises one or more extraction internal standards, wherein at least one of the extraction internal standards comprises a polypeptide standard. The extraction internal standards may be placed in a variety of configurations on the blood spot card such that once the blood sample is applied and allowed to dry, a portion of the blood spot card can be separated, such as by punching a chad from the blood spot card, the portion of the blood spot card comprising at least a portion of the blood sample and the one or more extraction internal standards. In some embodiments, the one or more extraction internal standards are deposited in a delimited zone of the blood spot card such that the one or more extraction internal standards are dried on the blood spot card prior to deposition of a blood sample. In some embodiments, the one or more extraction internal standards are deposited in a substantially uniform manner within the delimited zone. In some embodiments, the one or more extraction internal standards are deposited in a patterned manner within the delimited zone. In some embodiments, the delimited zone of a blood spot card comprises a known concentration of one or more extraction internal standards. In some embodiments, the method described herein comprise determining the total amount of a blood sample component and/or one or more extraction internal standards deposited on an absorbent or bibulous member, such as a blood spot card, based on the size of the one or more chads used to obtain polypeptide content for LC-MS analysis (e.g., a correction factor based on the size of the one or more chads and the size of a delimited area comprising a blood sample (or a portion thereof) and one or more extraction internal standards.

In some embodiments, the absorbent or bibulous member comprises a lateral flow element useful for performing a blood sample processing step. In some embodiments, the absorbent or bibulous member comprising a lateral flow element comprises (a) a delimited zone for deposition of a blood sample, and (b) a lateral flow element. In some embodiments, the absorbent or bibulous member comprises a zone, such as a delimited zone, wherein the component of the blood sample, such as a plasma or serum sample is obtained and allowed to dry thereon. In some embodiments, the zone comprising the plasma or serum sample is a portion of the lateral flow element.

FIG. 14B provides an example absorbent or bibulous member comprising a lateral flow element 1450. The absorbent or bibulous member 1400 comprises a delimited zone 1452 for deposition of a blood sample operably connected with a lateral flow zone 1454 configured for separation of one or more components of the blood sample. The absorbent or bibulous member 350 comprises a zone 1456 wherein the desired separated blood component can be obtained therefrom, e.g., a zone where a plasma or serum sample is dried thereon. The zone 1456 can be distal from and/or spaced apart from the delimited zone 1452. The absorbent or bibulous member 1450 further comprises a marking 1460 for the identification of the sample.

In some embodiments, the absorbent or bibulous member comprising a lateral flow element further comprises one or more extraction internal standards, wherein at least one of the extraction internal standards comprises a polypeptide standard. The extraction internal standards may be placed in a variety of configurations on the absorbent or bibulous member comprising a lateral flow element such that once the blood sample is applied and allowed to be processed and dry, a portion of the absorbent or bibulous member can be separated, such as by punching a chad from the absorbent or bibulous member, the portion of the absorbent or bibulous member comprising at least a portion of the blood sample and the one or more extraction internal standards. In some embodiments, the one or more extraction internal standards are deposited on a delimited zone configured for deposition of a blood sample. In some embodiments, the one or more extraction internal standards are deposited on a lateral flow element configured for processing a blood sample. In some embodiments, the one or more extraction internal standards are deposited on a zone configured for receipt of a component of a blood sample, such as a plasma or serum sample. Various combinations of the placement of the one or more extraction internal standards are encompassed by the description provided herein. In some embodiments, the absorbent or bibulous member comprising a lateral flow element comprises a plurality of populations of the one or more extraction internal standards, wherein the populations may of the extraction internal standards may be distinguished from one another (e.g., contain different polypeptide standards). In some embodiments, at least one population provides distinct information as compared to another population. For example, in some embodiments, the absorbent or bibulous member comprising a lateral flow element comprises a first population of the one or more extraction internal standards deposited on a delimited zone configured for deposition of a blood sample, wherein such population is useful for assessing sample migration to the zone for receipt of the processed sample. In some embodiments, the absorbent or bibulous member comprises a lateral flow element configured to separate whole blood into a portion of plasma, wherein the whole blood is deposited at the delimited zone and then a liquid portion of the whole blood laterally flows from the delimited zone to a distal zone, wherein the distal zone contains the portion of the plasma.

In some embodiments, the absorbent or bibulous member, including a single delimited zone of the absorbent or bibulous member, is configured to accept a blood sample having a volume of about 250 μL or less, such as about any of 225 μL or less, 200 μL or less, 175 μL or less, 150 μL or less, 125 μL or less, 100 μL or less, 75 μL or less, 50 μL or less, or 25 μL or less. In some embodiments, the absorbent or bibulous member, including a single delimited zone of the absorbent or bibulous member, is configured to accept a blood sample having a volume of about any of 250 μL, 225 μL, 200 μL, 175 μL, 150 μL, 125 μL, 100 μL, 75 μL, 50 μL, or 25 μL. In some embodiments, the absorbent or bibulous member, including a single delimited zone of the absorbent or bibulous member, is configured to accept a blood sample obtained from lancet of an individual, such as from a finger prick or a heel prick. In some embodiments, the absorbent or bibulous member comprises 1 to 10 delimited zoned configured for deposition of a blood sample. In some embodiments, the absorbent or bibulous member comprises 1 to 10 delimited zoned configured for deposition of a blood sample, wherein each delimited zone is configured to accept a blood sample having a volume of about 250 μL or less.

### II. Extraction Internal Standards and Uses Thereof

In certain aspects, provided herein are extraction internal standards suitable for use with the absorbent or bibulous members, such as a blood spot card, and methodology described herein. In some embodiments, provided is one or more extraction internal standards, wherein at least one of the one or more extraction internal standards comprises a polypeptide standard. In some embodiments, all of the one or more extraction internal standards are polypeptide standards. In some embodiments, a single extraction internal standard is used in the compositions and methods described herein, wherein the extraction internal standard is a polypeptide standard. In some embodiments, a plurality of extraction internal standards are used in the compositions and methods described herein, wherein at least one of the extraction internal standards is a polypeptide standard. As described herein, the polypeptide standards have a known composition, known amount, and known location on the absorbent or bibulous member (e.g., prior to deposition of a blood sample) such that the polypeptide standard serves as a reference point for one or more processes involved with analyzing the components of the blood sample using an LC-MS technique. In some embodiments, the polypeptide standard having a known location will move, such as due to placement of a blood sample on an absorbent or bibulous member. For example, as described in more detail herein, in the some embodiments, the polypeptide standard is useful for determining one or more of an extraction efficiency, a digestion efficiency, a sample migration pattern, or quantification of a polypeptide.

In some embodiments, the one or more extraction internal standards comprise a plurality of polypeptide standards, wherein at least two polypeptide standards of the plurality have different amino acid lengths. In some embodiments, the at least two polypeptide standards of the plurality are different in length by 1 or more amino acid, such as any of 5 or more, 10 or more, 20 or more, 30 or more, 40 or more, 50 or more, 60 or more, 70 or more, 80 or more, 90 or more, 100 or more, 150 or more, 200 or more, 250 or more, 300 or more, 400 or more, 500 or more, 600 or more, 700 or more, 800 or more, 900 or more, 1000 or more, 1100 or more, 1200 or more, 1300 or more, 1400 or more, 15000 or more, 1600 or more, 1700 or more, 1800 or more, 1900 or more, or 2000 or more amino acids. In some embodiments, the at least two polypeptide standards of the plurality are different in length by about any of 1, 5, 10, 20, 30, 40, 50, 60, 70, 80, 90, 100, 150, 200, 250, 300, 400, 500, 600, 700, 800, 900, 1000, 1100, 1200, 1300, 1400, 15000, 1600, 1700, 1800, 1900, or 2000 amino acids.

In some embodiments, the polypeptide standard has an amino acid length of at least about 4 amino acids to about 200 amino acids, such as any of about 5 amino acids to about 1900 amino acids, about 10 amino acids to about 1800 amino acids, about 20 amino acids to about 1700 amino acids, about 30 amino acids to about 1600 amino acids, about 40 amino acids to about 1500 amino acids, about 50 amino acids to about 1400 amino acids, about 60 amino acids to about 1300 amino acids, about 70 amino acids to about 1200 amino acids, about 80 amino acids to about 1100 amino acids, about 90 amino acids to about 1000 amino acids, about 100 amino acids to about 900 amino acids, about 200 amino acids to about 800 amino acids, about 300 amino acids to about 700 amino acids, about 400 amino acids to about 600 amino acids, or about 500 amino acids to about 750 amino acids. In some embodiments, the polypeptide standard has an amino acid length of at least about 4 amino acids, such as at least about any of 10 amino acids, 20 amino acids, 30 amino acids, 40 amino acids, 50 amino acids, 75 amino acids, 100 amino acids, 150 amino acids, 200 amino acids, 250 amino acids, 300 amino acids, 350 amino acids, 400 amino acids, 450 amino acids, 500 amino acids, 550 amino acids, 600 amino acids, 650 amino acids, 700 amino acids, 750 amino acids, 800 amino acids, 850 amino acids, 900 amino acids, 950 amino acids, 1000 amino acids, 1100 amino acids, 1200 amino acids, 1300 amino acids, 1400 amino acids, 1500 amino acids, 1600 amino acids, 1700 amino acids, 1800 amino acids, 1900 amino acids, or 2000 amino acids.

In some embodiments, at least one polypeptide standard of the one or more extraction internal standards comprises at least one internal enzymatic cleavage site, such as a cleavage site for trypsin or LysC. In some embodiments, the internal enzymatic cleavage site of the at least one polypeptide standard is cleaved during the provided method, resulting in two or more polypeptide fragments, wherein the two or more polypeptide fragments are shorter than the uncleaved polypeptide standard. In some embodiments, the cleaved polypeptide fragments comprise a C-terminal arginine or lysine. In some embodiments, the at least one polypeptide standard comprises a polypeptide comprising a C-terminal arginine or lysine.

In some embodiments, the one or more extraction internal standards comprise a plurality of polypeptide standards, wherein at least two of the plurality of polypeptide standards have different net hydrophobicities. In some embodiments, the net hydrophobicity is based on a partition coefficient analysis. In some embodiments, the net hydrophobicity is based on an octanol and water partition coefficient. In some embodiments, the net hydrophobicity is based on a computational tool. In some embodiments, the plurality of polypeptide standards having different net hydrophobicities comprises a hydrophobicity range. In some embodiments, the plurality of polypeptide standards comprise a hydrophobicity range as defined by the Grand average of hydropathicity index (GRAVY). In some embodiments, a hydrophobicity range as defined by GRAVY is about −0.5 to about 1. In some embodiments, a hydrophobicity range as defined by GRAVY is about −1 to about 2, about −0.8 to about 1.8, about −0.6 to about 1.6, about −0.4 to about 1.4, about −0.2 to about 1.2, about 0 to about 1, about 0.2 to about 0.8, about 0.4 to about 0.6, or about 0.5 to about 0.9. In some embodiments, a hydrophobicity range as defined by GRAVY is −1 to 2, −0.8 to 1.8, −0.6 to 1.6, −0.4 to 1.4, −0.2 to 1.2, 0 to 1, 0.2 to 0.8, 0.4 to 0.6, or 0.5 to 0.9. In some embodiments, a hydrophobicity range as defined by GRAVY is −0.5 to 1.

In some embodiments, the at least one polypeptide standard of the one or more extraction internal standards comprises a sequence that is non-homologous to an endogenous polypeptide of an individual from which the blood sample originates. In some embodiments, the at least one polypeptide standard comprises an amino acid sequence that does not have homology to a peptide derived from the human proteome. In some embodiments, the at least one polypeptide standard comprises an amino acid sequence derived from a bacterial, a fungal, an insect, a plant, or a non-human animal proteome. In some embodiments, the at least one polypeptide standard comprises an amino acid sequence of unknown origin (e.g., a sequence not found in known organisms or exogenous).

In some embodiments, the at least one polypeptide standard of the one or more extraction internal standards is a synthetic polypeptide. In some embodiments, the synthetic peptide is one or more of SEQ ID NOs: 21 and 22. In some embodiments, the at least one polypeptide standard comprises a sequence that is an analog of an endogenous polypeptide of an individual from which the blood sample originates. In some embodiments, the at least one polypeptide standard comprises an unnatural amino acid. In some embodiments, the unnatural amino acid comprises a fluorescent moiety, a functional moiety, and/or a reactive moiety.

In some embodiments, the at least one polypeptide standard of the one or more extraction internal standards is a synthetic polypeptide, wherein the at least one polypeptide standard comprises a stable heavy isotope label. In some embodiments, the stable heavy isotope label of the at least one polypeptide standard comprises one or more heavy labeled arginine, lysine, leucine, valine, or a combination thereof. In some embodiments, the stable heavy isotope label of the at least one polypeptide standard comprises 2-plex labeling, 3-plex labeling, or higher multiplex labeling. In some embodiments, the stable heavy isotope label of the at least one polypeptide standard is produced using stable isotope labeling by amino acids in cell culture (SILAC). In some embodiments, the analog of the endogenous polypeptide is a stable heavy isotope labeled analog polypeptide. In some embodiments, the non-homologous polypeptide is a stable heavy isotope labeled non-homologous polypeptide. In some embodiments, the non-human polypeptide is a stable heavy isotope labeled non-human polypeptide.

In some embodiments, the at least one polypeptide standard of the one or more extraction internal standards is a recombinantly expressed polypeptide. In some embodiments, the recombinantly expressed polypeptide comprises an amino acid sequence range from about 4 amino acids to about 2000 amino acids, about 5 amino acids to about 1900 amino acids, about 10 amino acids to about 1800 amino acids, about 20 amino acids to about 1700 amino acids, about 30 amino acids to about 1600 amino acids, about 40 amino acids to about 1500 amino acids, about 50 amino acids to about 1400 amino acids, about 60 amino acids to about 1300 amino acids, about 70 amino acids to about 1200 amino acids, about 80 amino acids to about 1100 amino acids, about 90 amino acids to about 1000 amino acids, about 100 amino acids to about 900 amino acids, about 200 amino acids to about 800 amino acids, about 300 amino acids to about 700 amino acids, about 400 amino acids to about 600 amino acids, or about 500 amino acids to about 750 amino acids. In some embodiments, the recombinantly expressed polypeptide comprises at least one internal enzymatic cleavage site. In some embodiments, the recombinantly expressed polypeptide of the one or more extraction internal standards have different net hydrophobicities. In some embodiments, the recombinantly expressed polypeptide comprises a sequence that is non-homologous to an endogenous polypeptide of an individual from which the blood sample originates. In some embodiments, the recombinantly expressed polypeptide comprises a sequence that does not have homology to a peptide derived from the human proteome. In some embodiments, the recombinantly expressed polypeptide comprises an analog sequence, wherein one or more amino acids are unnatural or derivatives of the endogenous sequence. In some embodiments, the recombinantly expressed polypeptide is a synthetic polypeptide. In some embodiments, the recombinantly expressed polypeptide comprises a stable heavy isotope label.

In some embodiments, the at least one polypeptide standard of the one or more extraction internal standards is a glycopolypeptide. In some embodiments, the glycopolypeptide comprises an amino acid sequence range from about 4 amino acids to about 2000 amino acids, about 5 amino acids to about 1900 amino acids, about 10 amino acids to about 1800 amino acids, about 20 amino acids to about 1700 amino acids, about 30 amino acids to about 1600 amino acids, about 40 amino acids to about 1500 amino acids, about 50 amino acids to about 1400 amino acids, about 60 amino acids to about 1300 amino acids, about 70 amino acids to about 1200 amino acids, about 80 amino acids to about 1100 amino acids, about 90 amino acids to about 1000 amino acids, about 100 amino acids to about 900 amino acids, about 200 amino acids to about 800 amino acids, about 300 amino acids to about 700 amino acids, about 400 amino acids to about 600 amino acids, or about 500 amino acids to about 750 amino acids. In some embodiments, the glycopolypeptide comprises at least one internal enzymatic cleavage site. In some embodiments, the glycopolypeptide of the one or more extraction internal standards have different net hydrophobicities. In some embodiments, the glycopolypeptide comprises a sequence that is non-homologous to an endogenous polypeptide of an individual from which the blood sample originates. In some embodiments, the glycopolypeptide comprises a sequence that does not have homology to a peptide derived from the human proteome. In some embodiments, the glycopolypeptide comprises an analog sequence, wherein one or more amino acids are unnatural or derivatives of the endogenous sequence. In some embodiments, the glycopolypeptide is a synthetic polypeptide. In some embodiments, the glycopolypeptide comprises a stable heavy isotope label.

In some embodiments, the at least one polypeptide standard of the one or more extraction internal standards is a polypeptide that does not substantially interact with hemoglobin. In some embodiments, hemoglobin does not substantially impact the extraction of the polypeptide. In some embodiments, hemoglobin does not substantially impact the processing of the polypeptide. In some embodiments, hemoglobin does not substantially impact the digestion of the polypeptide.

In some embodiments, the at least one polypeptide standard of the one or more extraction internal standards comprises a contiguous sequence from SEQ ID NOs: 14-20. In some embodiments, the at least one polypeptide standard of the one or more extraction internal standards comprises at least a contiguous 4 amino acid sequence from SEQ ID NOs: 14-20. In some embodiments, the at least one polypeptide standard of the one or more extraction internal standards comprises at least a contiguous sequence of about 4, about 5, about 6, about 7, about 8, about 9, about 10, about 11, about 12, about 13, about 14, about 15, about 16, about 17, about 18, about 19, about 20, about 30, about 40, about 50, about 60, about 70, about 80, about 90, or about 100 acids from SEQ ID NOs: 14-20. In some embodiments, the at least one polypeptide standard of the one or more extraction internal standards comprises one or more polypeptides human apolipoprotein C-III (APOC3) of SEQ ID NO: 14, human alpha-2-macroglobulin (A2MG) of SEQ ID NO: 15, human ceruloplasmin (CERU) of SEQ ID NO: 16, human alpha-1-acid glycoprotein 1 (AGP1) of SEQ ID NO: 17, human haptoglobin (HPT) of SEQ ID NO: 18, human hemopexin (HEMO) of SEQ ID NO: 19, or human beta-2-glycoprotein 1 (APOH) of SEQ ID NO: 20. In some embodiments, the at least one polypeptide standard of the one or more extraction internal standards is APOC3, for example SEQ ID NO: 14. In some embodiments, the at least one polypeptide standard of the one or more extraction internal standards is A2MG, for example SEQ ID NO: 215 In some embodiments, the at least one polypeptide standard of the one or more extraction internal standards is CERU, for example SEQ ID NO: 16. In some embodiments, the at least one polypeptide standard of the one or more extraction internal standards is AGP1, for example SEQ ID NO: 17. In some embodiments, the at least one polypeptide standard of the one or more extraction internal standards is HPT, for example SEQ ID NO: 18. In some embodiments, the at least one polypeptide standard of the one or more extraction internal standards is HEMO, for example SEQ ID NO: 19. In some embodiments, the at least one polypeptide standard of the one or more extraction internal standards is APOH, for example SEQ ID NO: 20. In some embodiments, the at least one polypeptide standard of the one or more extraction internal standards has at least 85%, at least 90%, at least 95%, at least 96%, at least 97%, at least 98%, or at least 99% sequence identity to one or more of SEQ ID NOs: 14-20. In some embodiments, the at least one polypeptide standard of the one or more extraction internal standards has 85%, 86%, 87%, 88%, 89%, 90%, 91%, 92%, 93%, 94%, 95%, 96%, 97%, 98%, or 99% sequence identity to one or more of SEQ ID NOs: 14-20. In some embodiments, the at least one polypeptide standard of the one or more extraction internal standards is any of the polypeptides described herein, wherein the polypeptide further comprises one or more of an amino acid sequence range from about 4 amino acids to about 2000 amino acids, at least one internal enzymatic cleavage site, a unique net hydrophobicity, a sequence that is non-homologous to an endogenous polypeptide of an individual from which the blood sample originates, a sequence that does not have homology to a peptide derived from the human proteome, an analog sequence, wherein one or more amino acids are unnatural or derivatives of the endogenous sequence, a stable heavy isotope label, a polypeptide that does not substantially interact with hemoglobin, a recombinantly expressed polypeptide, or a glycopolypeptide. In an embodiment, the proteins of SEQ ID NOs: 14-20 are heavy labeled proteins where all arginines and lysines are replaced with stable isotope labeled lysine and arginine. The proteins are labeled with a stable isotopes so that they can be monitored without confusing them for the endogenous proteins in the blood sample. Such heavy labeled glycoproteins could be produced through recombinant expression in an orthologous system, cultured in SILAC supplemented media. In an embodiment, the proteins of SEQ ID NOs: 14-20 have all of their lysines and arginines heavy isotope labeled and deposited on a filter card. When blood is applied, a portion of the labeled proteins of SEQ ID NOs: 14-20 is extracted from the filter paper or DBS and then digested with a proteolytic enzyme. A resulting peptide produced from the enzymatic digestion having the isotope label from each of the proteins of SEQ ID NOs: 14-20 can be monitored with multiple reaction monitoring (MRM) for determining the extraction efficiency and/or tryptic efficiency for each of the labeled proteins. Assuming that the proteins of SEQ ID NOS: 14-20 are efficiently digested, they can be used to determine the extraction efficiency. Alternatively, if the proteins of SEQ ID NOS: 14-20 are not completely digested, then it can be assumed that the digestion efficiency of the proteins of SEQ ID NOS: 14-20 are the same as the proteins in the sample allowing the proteins of SEQ ID NOS: 14-20 to be used for determining the extraction efficiency. The labeled isotope allows the tryptic peptides generated from the proteins of SEQ ID NOs: 14-20 to be distinguished from naturally occurring tryptic peptides. In an embodiment, peptides of SEQ ID NOs: 21 and 22 can represent an exogenous or non-human peptide not normally found in human subjects and be deposited on a filter paper or DBS. Since SEQ ID NOs: 21 and 22 are not normally found in human subjects, they can be used for determining the extraction efficiency. The peptides of SEQ ID NOS: 21 and 22 are smaller molecules than the proteins of SEQ ID NOS: 14-20 causing the peptides of SEQ ID NOS: 21 and 22 to potentially have less variations in extraction efficiency from the dried blood spot than the proteins of SEQ ID NOS: 14-20.

TABLE 13
Sources of example polypeptide standards

| SEQ | | |
|---|---|---|
| ID | | |
| NO | Sequence | Origin |
| 14 | MQPRVLLVVALLALLASARASEAEDASLLSFMQGY | Human |
| | MKHATKTAKDALSSVQESQVAQQARGWVTDGFSSL | Apolipoprotein |
| | KDYWSTVKDKFSEFWDLDPEVRPTSAVAA | C-III |
| | | (APOC3) |
| 15 | MGKNKLLHPSLVLLLLVLLPTDASVSGKPQYMVLV | Human |
| | PSLLHTETTEKGCVLLSYLNETVTVSASLESVRGN | Alpha-2 |
| | RSLFTDLEAENDVLHCVAFAVPKSSSNEEVMFLTV | macroglobulin |
| | QVKGPTQEFKKRTTVMVKNEDSLVFVQTDKSIYKP | (A2MG) |
| | GQTVKFRVVSMDENFHPLNELIPLVYIQDPKGNRI | |
| | AQWQSFQLEGGLKQFSFPLSSEPFQGSYKVVVQKK | |
| | SGGRTEHPFTVEEFVLPKFEVQVTVPKIITILEEE | |
| | MNVSVCGLYTYGKPVPGHVTVSICRKYSDASDCHG | |
| | EDSQAFCEKFSGQLNSHGCFYQQVKTKVFQLKRKE | |
| | YEMKLHTEAQIQEEGTVVELTGRQSSEITRTITKL | |
| | SFVKVDSHFRQGIPFFGQVRLVDGKGVPIPNKVIF | |
| | IRGNEANYYSNATTDEHGLVQFSINTTNVMGTSLT | |
| | VRVNYKDRSPCYGYQWVSEEHEEAHHTAYLVFSPS | |
| | KSFVHLEPMSHELPCGHTQTVQAHYILNGGTLLGL | |
| | KKLSFYYLIMAKGGIVRTGTHGLLVKQEDMKGHFS | |
| | ISIPVKSDIAPVARLLIYAVLPTGDVIGDSAKYDV | |
| | ENCLANKVDLSFSPSQSLPASHAHLRVTAAPQSVC | |
| | ALRAVDQSVLLMKPDAELSASSVYNLLPEKDLTGF | |
| | PGPLNDQDNEDCINRHNVYINGITYTPVSSTNEKD | |
| | MYSFLEDMGLKAFTNSKIRKPKMCPQLQQYEMHGP | |
| | EGLRVGFYESDVMGRGHARLVHVEEPHTETVRKYF | |
| | PETWIWDLVVVNSAGVAEVGVTVPDTITEWKAGAF | |
| | CLSEDAGLGISSTASLRAFQPFFVELTMPYSVIRG | |
| | EAFTLKATVLNYLPKCIRVSVQLEASPAFLAVPVE | |
| | KEQAPHCICANGRQTVSWAVTPKSLGNVNFTVSAE | |
| | ALESQELCGTEVPSVPEHGRKDTVIKPLLVEPEGL | |
| | EKETTFNSLLCPSGGEVSEELSLKLPPNVVEESAR | |
| | ASVSVLGDILGSAMQNTQNLLQMPYGCGEQNMVLF | |
| | APNIYVLDYLNETQQLTPEIKSKAIGYLNTGYQRQ | |
| | LNYKHYDGSYSTFGERYGRNQGNTWLTAFVLKTFA | |
| | QARAYIFIDEAHITQALIWLSQRQKDNGCFRSSGS | |
| | LLNNAIKGGVEDEVTLSAYITIALLEIPLTVTHPV | |
| | VRNALFCLESAWKTAQEGDHGSHVYTKALLAYAFA | |
| | LAGNQDKRKEVLKSLNEEAVKKDNSVHWERPQKPK | |
| | APVGHFYEPQAPSAEVEMTSYVLLAYLTAQPAPTS | |
| | EDLTSATNIVKWITKQQNAQGGFSSTQDTVVALHA | |
| | LSKYGAATFTRTGKAAQVTIQSSGTFSSKFQVDNN | |
| | NRLLLQQVSLPELPGEYSMKVTGEGCVYLQTSLKY | |
| | NILPEKEEFPFALGVQTLPQTCDEPKAHTSFQISL | |
| | SVSYTGSRSASNMAIVDVKMVSGFIPLKPTVKMLE | |
| | RSNHVSRTEVSSNHVLIYLDKVSNQTLSLFFTVLQ | |
| | DVPVRDLKPAIVKVYDYYETDEFAIAEYNAPCSKD | |
| | LGNA | |
| 16 | MKILILGIFLFLCSTPAWAKEKHYYIGIIETTWDY | Human |
| | ASDHGEKKLISVDTEHSNIYLQNGPDRIGRLYKKA | ceruloplasmin |
| | LYLQYTDETFRTTIEKPVWLGFLGPIIKAETGDKV | (CERU) |
| | YVHLKNLASRPYTFHSHGITYYKEHEGAIYPDNTT | |
| | DFQRADDKVYPGEQYTYMLLATEEQSPGEGDGNCV | |
| | TRIYHSHIDAPKDIASGLIGPLIICKKDSLDKEKE | |
| | KHIDREFVVMFSVVDENFSWYLEDNIKTYCSEPEK | |
| | VDKDNEDFQESNRMYSVNGYTFGSLPGLSMCAEDR | |
| | VKWYLFGMGNEVDVHAAFFHGQALTNKNYRIDTIN | |
| | LFPATLFDAYMVAQNPGEWMLSCQNLNHLKAGLQA | |
| | FFQVQECNKSSSKDNIRGKHVRHYYIAAEEIIWNY | |
| | APSGIDIFTKENLTAPGSDSAVFFEQGTTRIGGSY | |
| | KKLVYREYTDASFTNRKERGPEEEHLGILGPVIWA | |
| | EVGDTIRVTFHNKGAYPLSIEPIGVRFNKNNEGTY | |
| | YSPNYNPQSRSVPPSASHVAPTETFTYEWTVPKEV | |
| | GPTNADPVCLAKMYYSAVDPTKDIFTGLIGPMKIC | |
| | KKGSLHANGRQKDVDKEFYLFPTVFDENESLLLED | |
| | NIRMFTTAPDQVDKEDEDFQESNKMHSMNGFMYGN | |
| | QPGLTMCKGDSVVWYLFSAGNEADVHGIYFSGNTY | |
| | LWRGERRDTANLFPQTSLTLHMWPDTEGTFNVECL | |
| | TTDHYTGGMKQKYTVNQCRRQSEDSTFYLGERTYY | |
| | IAAVEVEWDYSPQREWEKELHHLQEQNVSNAFLDK | |
| | GEFYIGSKYKKVVYRQYTDSTFRVPVERKAEEEHL | |
| | GILGPQLHADVGDKVKIIFKNMATRPYSIHAHGVQ | |
| | TESSTVTPTLPGETLTYVWKIPERSGAGTEDSACI | |
| | PWAYYSTVDQVKDLYSGLIGPLIVCRRPYLKVFNP | |
| | RRKLEFALLFLVFDENESWYLDDNIKTYSDHPEKV | |
| | NKDDEEFIESNKMHAINGRMFGNLQGLTMHVGDEV | |
| | NWYLMGMGNEIDLHTVHFHGHSFQYKHRGVYSSDV | |
| | FDIFPGTYQTLEMFPRTPGIWLLHCHVTDHIHAGM | |
| | ETTYTVLQNEDTKSG | |
| 17 | MALSWVLTVLSLLPLLEAQIPLCANLVPVPITNAT | Human |
| | LDQITGKWFYIASAFRNEEYNKSVQEIQATFFYFT | Alpha-1- |
| | PNKTEDTIFLREYQTRQDQCIYNTTYLNVQRENGT | acid |
| | ISRYVGGQEHFAHLLILRDTKTYMLAFDVNDEKNW | glycoprotein 1 |
| | GLSVYADKPETTKEQLGEFYEALDCLRIPKSDVVY | (AGP1) |
| | TDWKKDKCEPLEKQHEKERKQEEGES | |
| 18 | MSALGAVIALLLWGQLFAVDSGNDVTDIADDGCPK | Human |
| | PPEIAHGYVEHSVRYQCKNYYKLRTEGDGVYTLND | Haptoglobin |
| | KKQWINKAVGDKLPECEADDGCPKPPEIAHGYVEH | (HPT) |
| | SVRYQCKNYYKLRTEGDGVYTLNNEKQWINKAVGD | |
| | KLPECEAVCGKPKNPANPVQRILGGHLDAKGSFPW | |
| | QAKMVSHHNLTTGATLINEQWLLTTAKNLFLNHSE | |
| | NATAKDIAPTLTLYVGKKQLVEIEKVVLHPNYSQV | |
| | DIGLIKLKQKVSVNERVMPICLPSKDYAEVGRVGY | |
| | VSGWGRNANFKFTDHLKYVMLPVADQDQCIRHYEG | |
| | STVPEKKTPKSPVGVQPILNEHTFCAGMSKYQEDT | |
| | CYGDAGSAFAVHDLEEDTWYATGILSFDKSCAVAE | |
| | YGVYVKVTSIQDWVQKTIAEN | |
| 19 | MARVLGAPVALGLWSLCWSLAIATPLPPTSAHGNV | Human |
| | AEGETKPDPDVTERCSDGWSFDATTLDDNGTMLFF | Hemopexin |
| | KGEFVWKSHKWDRELISERWKNFPSPVDAAFRQGH | (HEMO) |
| | NSVFLIKGDKVWVYPPEKKEKGYPKLLQDEFPGIP | |
| | SPLDAAVECHRGECQAEGVLFFQGDREWFWDLATG | |
| | TMKERSWPAVGNCSSALRWLGRYYCFQGNQFLRFD | |
| | PVRGEVPPRYPRDVRDYFMPCPGRGHGHRNGTGHG | |
| | NSTHHGPEYMRCSPHLVLSALTSDNHGATYAFSGT | |
| | HYWRLDTSRDGWHSWPIAHQWPQGPSAVDAAFSWE | |
| | EKLYLVQGTQVYVFLTKGGYTLVSGYPKRLEKEVG | |
| | TPHGIILDSVDAAFICPGSSRLHIMAGRRLWWLDL | |
| | KSGAQATWTELPWPHEKVDGALCMEKSLGPNSCSA | |
| | NGPGLYLIHGPNLYCYSDVEKLNAAKALPQPQNVT | |
| | SLLGCTH | |
| 20 | MISPVLILFSSFLCHVAIAGRTCPKPDDLPFSTVV | Human |
| | PLKTFYEPGEEITYSCKPGYVSRGGMRKFICPLTG | Beta-2- |
| | LWPINTLKCTPRVCPFAGILENGAVRYTTFEYPNT | glycoprotein 1 |
| | ISFSCNTGFYLNGADSAKCTEEGKWSPELPVCAPI | (APOH) |
| | ICPPPSIPTFATLRVYKPSAGNNSLYRDTAVFECL | |
| | PQHAMFGNDTITCTTHGNWTKLPECREVKCPFPSR | |
| | PDNGFVNYPAKPTLYYKDKATFGCHDGYSLDGPEE | |
| | IECTKLGNWSAMPSCKASCKVPVKKATVVYQGERV | |
| | KIQEKFKNGMLHGDKVSFFCKNKEKKCSYTEDAQC | |
| | IDGTIEVPKCFKEHSSLAFWKTDASDVKPC | |
| 21 | RPAIAINNPYVPR | |
| 22 | FFVAPFPEVFGK | |

In some embodiments, the at least one polypeptide standard of the one or more extraction internal standards is present in a known amount on the absorbent or bibulous member, such as a blood spot card. In some embodiments, the at least one polypeptide standard is present in a known amount on the absorbent or bibulous member, such as a blood spot card, prior to contact the absorbent or bibulous member with a blood sample. In some embodiments, the known amount of each of the one or more extraction internal standards, such as a polypeptide standard, is about 0.05 ppm to about 5 ppm. In some embodiments, the known amount of each of the one or more extraction internal standards, such as a polypeptide standard, is about 0.02 ppm to about 10 ppm, about 0.05 ppm to about 9 ppm, about 0.1 ppm to about 9 ppm, about 0.2 ppm to about 8 ppm, about 0.3 ppm to about 7 ppm, about 0.4 ppm to about 6 ppm, about 0.5 ppm to about 5 ppm, about 0.6 ppm to about 4 ppm, about 0.7 ppm to about 3 ppm, about 0.8 ppm to about 2 ppm, or about 0.9 ppm to about 1 ppm.

In some embodiments, the one or more extraction internal standards are deposited and dried on the absorbent or bibulous member, such as a blood spot card, within an area having a surface area of about 1,000 mm² or less. In some embodiments, the area for depositing the one or more extraction internal standards, such as a polypeptide standard, has a surface area of about 1,000 mm² or less, about 950 mm² or less, about 900 mm² or less, about 850 mm² or less, about 800 mm² or less, about 750 mm² or less, about 700 mm² or less, about 650 mm² or less, about 600 mm² or less, about 550 mm² or less, about 500 mm² or less, about 450 mm² or less, about 400 mm² or less, about 350 mm² or less, about 300 mm² or less, about 250 mm² or less, about 200 mm² or less, about 150 mm² or less, about 100 mm² or less, or about 50 mm² or less. In some embodiments, the area for depositing the one or more extraction internal standards, such as a polypeptide standard, has a surface area of 1,000 mm² or less, 950 mm² or less, 900 mm² or less, 850 mm² or less, 800 mm² or less, 750 mm² or less, 700 mm² or less, 650 mm² or less, 600 mm² or less, 550 mm² or less, 500 mm² or less, 450 mm² or less, 400 mm² or less, 350 mm² or less, 300 mm² or less, 250 mm² or less, 200 mm² or less, 150 mm² or less, 100 mm² or less, or 50 mm² or less. In some embodiments, the less can represent the lower limit of a range for surface area as described above where the lower limit is about 2 mm², 4 mm², 6 mm², 8 mm², or 10 mm².

In some embodiments, the one or more extraction internal standard, such as a polypeptide standard, are deposited and dried on the absorbent or bibulous member, such as a blood spot card, within the delimited zone. In some embodiments, the delimited zone for depositing and drying the one or more extraction internal standard is a defined area, such as marked by a visible line or dash. In some embodiments, the delimited zone is about 1,000 mm² or less. In some embodiments, the delimited zone is any size described herein. In some embodiments, the delimited zone contains a known amount of each of the one or more extraction internal standards, such as a polypeptide standard. In some embodiments, the delimited zone contains about 0.05 ppm to about 5 ppm of each of the one or more extraction internal standards.

III. Methods Associated with the Absorbent or Bibulous Members Described Herein

In certain aspects, provided herein are methods associated with the absorbent or bibulous members described herein, including absorbent or bibulous members comprising one or more extraction internal standards, wherein at least one of the extraction internal standards is a polypeptide standard.

In some embodiments, provided is a method of making an absorbent or bibulous member described herein, such as a blood spot card, comprising one or more extraction internal standards, wherein at least one of the extraction internal standards is a polypeptide standard, the method comprising depositing the one or more extraction internal standards on the absorbent or bibulous member. In some embodiments, the method comprises drying the one or more extraction internal standards following deposition on the absorbent or bibulous member.

In some embodiments, provided is a method for obtaining an absorbent or bibulous member, such as a blood spot card, comprising a blood sample deposited thereon, the method comprising providing the absorbent or bibulous member comprising one or more extraction internal standards, wherein at least one of the extraction internal standards is a polypeptide standard, and providing instructions for the deposition of the blood sample onto the absorbent or bibulous member. In some embodiments, the method comprises depositing the blood sample onto the absorbent or bibulous member. In some embodiments, the method comprises mailing, such as via standard mail with a government regulated mail carrier, e.g., The United States Postal Service, the absorbent or bibulous member comprising the blood sample and the one or more extraction internal standards.

In some embodiments, provided is a method of extracting at least a portion of a plurality of polypeptides and one or more extraction internal standards, wherein the one or more extraction internal standards comprises a polypeptide standard, from an absorbent or bibulous member, such as a blood spot card. In some embodiments, the method comprises: separating one or more portions of the blood spot card from the blood spot card, wherein the one or more portions of the absorbent or bibulous member comprise at least a portion of the blood sample and the one or more extraction internal standards; extracting at least the portion of the plurality of polypeptides and the one or more extraction internal standards from the one or more portions of the absorbent or bibulous member into an extraction solution; and precipitating at least the portion of the plurality of polypeptides and the one or more extraction internal standards to obtain the extracted sample. In some embodiments, the separating the one or more portions of the absorbent or bibulous member comprises punching the one or more portion of the absorbent or bibulous member using a punching device. In some embodiments, the separated portion of the absorbent or bibulous member is referred to as a chad. In some embodiments, the extracting may include a thermal denaturation step and as water bath sonication in order to mix and promote diffusion of glycoproteins out of absorbent or bibulous member and into the extraction solution. The extraction solution may include buffer such as 50 mM ammonium bicarbonate, and a reducing agent such as 25 mM DTT to help promote denaturation and extraction of the proteins.

In some embodiments, each of the one or more portions separated from the absorbent or bibulous member have a surface area of about 2 mm² to about 100 mm². In some embodiments, each of the one or more portions separated from the absorbent or bibulous member have a surface area of about any of 1 mm², 2 mm², 3 mm², 4 mm², 5 mm², 6 mm², 7 mm², 8 mm², 9 mm², 10 mm², 15 mm², 20 mm², 25 mm², 30 mm², 35 mm², 40 mm², 45 mm², 50 mm², 55 mm², 60 mm², 65 mm², 70 mm², 75 mm², 80 mm², 85 mm², 90 mm², 95 mm², or 100 mm².

In some embodiments, the precipitating at least the portion of the plurality of polypeptides and the one or more extraction internal standards comprises subjecting the at least the portion of the plurality of polypeptides and the one or more extraction internal standards to an organic solvent, such as either ethanol, methanol, or acetone.

In some embodiments, the method further comprises adding a solution, such as a buffer, e.g., ammonium bicarbonate, to the extracted sample to resolubilize polypeptide content therein prior to subjecting the extracted sample or the derivative thereof to the proteolytic digestion technique.

In some embodiments, provided herein is a method for performing a liquid chromatography-mass spectrometry (LC-MS) analysis of a proteolytic glycopeptide derived from a blood sample deposited on a delimited zone of an absorbent or bibulous member, such as a blood spot card, wherein the blood sample comprises a plurality of polypeptides comprising at least one glycoprotein, the method comprising extracting at least a portion of the plurality of polypeptides and one or more extraction internal standards from the absorbent or bibulous member to obtain an extracted sample, wherein the absorbent or bibulous member comprises the one or more extraction internal standards prior to deposition of the blood sample within the delimited zone, and wherein at least one of the one or more extraction internal standards comprises a polypeptide standard; subjecting the extracted sample or a derivative thereof to a proteolytic digestion technique to produce a proteolytically digested sample comprising the proteolytic glycopeptide; introducing at least a portion of the proteolytically digested sample to a liquid chromatography (LC) system of a LC-MS system; and performing the LC-MS analysis on at least the proteolytic glycopeptide and the one or more extraction internal standards.

In some embodiments, the performing the LC-MS analysis comprises measuring an abundance signal for the proteolytic glycopeptide and an abundance signal for the one or more extraction internal standards. In some embodiments, the performing the LC-MS analysis further comprises calculating a concentration of the proteolytic glycopeptide based on a concentration of the one or more extraction internal standards prior to deposition on the absorbent or bibulous member, such as a blood spot card, the abundance signal for the proteolytic glycopeptide, and the abundance signal for the one or more extraction internal standards.

In some embodiments, the method further comprises determining an extraction efficiency based on the LC-MS analysis of at least one of the one or more extraction internal standards. For example, in some embodiments, the extraction efficiency is based on an abundance signal for the one or more extraction internal standards as compared to a reference, such as a known amount of the one or more extraction internal standards deposited on the absorbent or bibulous member and/or comparison of two or more of the extraction internal standards.

In some embodiments, the method further comprises determining a digestion efficiency based on the LC-MS analysis of at least one of the one or more extraction internal standards. For example, in some embodiments, the digestion efficiency is based on an abundance signal for the one or more extraction internal standards, such as depletion of an abundance signal of a polypeptide standard comprising a protease cleavage site, and/or an increase in an abundance signal for one or more portions of a polypeptide standard, wherein the one or more portions of the polypeptide standard are a result of protease activity.

In some embodiments, the method further comprises assessing a sample migration pattern based on the LC-MS analysis of at least one of the one or more extraction internal standards. For example, in some embodiments, the sample migration pattern is based on an abundance signal for the one or more extraction internal standards obtained from a delimited zone of an absorbent or bibulous member, wherein the one or more extraction internal standards have known location prior to deposition of a blood sample on the absorbent or bibulous member. In some embodiments, the known location of the one or more extraction internal standards is within the delimited zone and the sample migration pattern provides information regarding the movement of the one or more extraction internal standards upon deposition of a blood sample. In some embodiments, the known location of the one or more extraction internal standards is outside of the delimited zone and the sample migration pattern provides information regarding the movement of the one or more extraction internal standards to the delimited zone upon deposition of a blood sample. In some embodiments, the movement of the one or more extraction internal standards provides information regarding the movement of a blood sample, or a component thereof, after being deposited onto an absorbent or bibulous member.

### D4. Methods for Proteolytically Digesting a Blood Sample Comprising a Glycoprotein

In certain aspects, provided herein are methods of proteolytically digesting a biological sample, such as an extract from a dried blood sample, comprising a glycoprotein. A dried blood spot sample can be punched to separate one or more discs which are then soaked in a buffer containing DTT at an elevated temperature of 95° C. and sonicated to extract the proteins and glycoproteins. In some embodiments, the extract can be in the form of a precipitate after the addition of either ethanol, methanol, or acetone. In some embodiments, the methods comprising subjecting the extract from the dried blood sample, or a portion thereof, to another thermal denaturation technique. Proteases are enzymes that cleave polypeptides at, generally, specific cleavage motifs. For example, trypsin is a serine protease that generally cleaves polypeptides at the carboxyl side (C-terminal side) of lysine and arginine residues. A glycan of a glycopeptide may present a steric hindrance to a protease, thereby inhibiting complete protease digestion of the extract from the dried blood sample, or a portion thereof, comprising a glycoprotein. Without being bound to this theory, it is believed that the methods taught herein improve polypeptide unfolding, such as linearization, and provide protease access to cleavage sites thereby providing methods for more complete proteolytic digestion of glycoproteins.

In some aspects, provided is a method comprising subjecting the extract from the dried blood sample, or portion thereof, to a thermal denaturation technique to produce a denatured sample.

In other aspects, provided is a method comprising subjecting the extract from the dried blood sample, or portion thereof, to a thermal denaturation technique to produce a denatured sample followed by a proteolytic digestion technique to produce a proteolytically digested sample comprising a proteolytic glycopeptide. In some embodiments, the method comprises quenching one or more proteases used in a proteolytic digestion technique prior to a downstream technique, such as LC-MS.

In other aspects, provided herein is a method comprising subjecting the extract from the dried blood sample, or portion thereof, to a thermal denaturation technique to produce a denatured sample; subjecting the denatured sample to a reduction technique to produce a reduced sample; subjecting the reduced sample to an alkylation technique to produce an alkylated sample; and subjecting the alkylated sample to a proteolytic digestion technique to produce a proteolytically digested sample comprising the proteolytic glycopeptide. In some embodiments, the method comprises quenching an alkylating agent used in the alkylation technique prior to subjecting an alkylated sample to a proteolytic digestion technique. In some embodiments, the method comprises quenching one or more proteases used in a proteolytic digestion technique prior to a downstream technique, such as LC-MS.

In some embodiments, the proteolytic digestion methods described herein produce a proteolytic digestion sample, including one comprising a proteolytic glycopeptide, having a digestion completion rate of at least about 70%, such as at least about any of 75%, 80%, 85%, 90%, 91%, 92%, 93%, 94%, 95%, 96%, 97%, 98%, or 99%. In some embodiments, the proteolytic digestion methods described herein produce a proteolytic digestion sample, including one comprising a proteolytic glycopeptide, wherein the sample volume loss is 10% or less, such as 9% or less, 8% or less, 7% or less, 6% or less, 5% or less, 4% or less, 3% or less, 2% or less, or 1% or less, based on all volumes added in producing the proteolytic digestion sample.

In the following sections, additional description of the various aspects of the proteolytic digestion techniques is provided. Such description in a modular fashion is not intended to limit the scope of the disclosure, and based on the teachings provided herein one of ordinary skill in the art will readily appreciate that certain modules can be integrated, at least in part. The section heading used herein are for organizational purposes only and are not to be construed as limiting the subject matter described.

### I. Thermal Denaturation Techniques

In certain aspects, the methods provided herein comprise performing a thermal denaturation technique. Thermal denaturation techniques, generally speaking, change certain polypeptides conformational structures, such as by unfolding and/or linearizing a polypeptide, to enable protease access to cleavage sites. Thermal denaturation techniques described herein comprise subjecting a sample, or a derivative thereof (e.g., a sample diluted with a buffer), to a thermal treatment of about 60° C. to about 100° C. for thermal denaturation incubation time of at least about 1 minute. In some embodiments, the thermal denaturation technique is not performed concurrently with a chemical denaturation technique, such as using high concentrations of denaturing agent, e.g., 6 M urea. In some embodiments, the method does not include use of a chemical denaturation technique.

In some embodiments, the thermal denaturation incubation time is performed at a temperature of about 60° C. to about 100° C., such as any of about 70° C. to about 100° C., about 80° C. to about 100° C., about 90° C. to about 100° C., about 95° C. to about 100° C., or about 85° C. to about 95° C. In some embodiments, the thermal denaturation incubation time is performed at a temperature of at least about 60° C., such as at least about any of 65° C., 70° C., 75° C., 80° C., 85° C., 90° C., 95° C., or 100° C. In some embodiments, the thermal denaturation incubation time is performed at a temperature of about 100° C. or less, such as about any of 95° C. or less, 90° C. or less, 85° C. or less, 80° C. or less, 75° C. or less, 70° C. or less, 65° C. or less, 60° C. or less. In some embodiments, the thermal denaturation incubation time is performed at a temperature of about any of 60° C., 65° C., 70° C., 75° C., 80° C., 85° C., 90° C., 91° C., 92° C., 93° C., 94° C., 95° C., 96° C., 97° C., 98° C., 99° C., or 100° C.

In some embodiments, the thermal denaturation incubation time is about 1 minute to about 15 minutes, such as any of about 1 minute to about 5 minutes, about 1 minute to about 10 minutes, about 2.5 minutes to about 7.5 minutes, or about 5 minutes to about 15 minutes. In some embodiments, the thermal denaturation incubation time is at least about 1 minute, such as at least about any of 1.5 minutes, 2 minutes, 2.5 minutes, 3 minutes, 3.5 minutes, 4 minutes, 4.5 minutes, 5 minutes, 5.5 minutes, 6 minutes, 6.5 minutes, 7 minutes, 7.5 minutes, 8 minutes, 8.5 minutes, 9 minutes, 9.5 minutes, 10 minutes, 11 minutes, 12 minutes, 13 minutes, 14 minutes, or 15 minutes. In some embodiments, the thermal denaturation incubation time is about 15 minutes or less, such as about any of 14 minutes or less, 13 minutes or less, 12 minutes or less, 11 minutes or less, 10 minutes or less, 9.5 minutes or less, 9 minutes or less, 8.5 minutes or less, 8 minutes or less, 7.5 minutes or less, 7 minutes or less, 6.5 minutes or less, 6 minutes or less, 5.5 minutes or less, 5 minutes or less, 4.5 minutes or less, 4 minutes or less, 3.5 minutes or less, 3 minutes or less, 2.5 minutes or less, 2 minutes or less, 1.5 minutes or less, or 1 minute or less. In some embodiments, the thermal denaturation incubation time is about any of 1 minute, 1.5 minutes, 2 minutes, 2.5 minutes, 3 minutes, 3.5 minutes, 4 minutes, 4.5 minutes, 5 minutes, 5.5 minutes, 6 minutes, 6.5 minutes, 7 minutes, 7.5 minutes, 8 minutes, 8.5 minutes, 9 minutes, 9.5 minutes, 10 minutes, 11 minutes, 12 minutes, 13 minutes, 14 minutes, or 15 minutes.

In some embodiments, the thermal denaturation technique comprises a thermal denaturation incubation time of about 1 minute to about 15 minutes, such as about any of 1 minute, 2 minutes, 3 minutes, 4 minutes, 5 minutes, 6 minutes, 7 minutes, 8 minutes, 9 minutes 10 minutes, 11 minutes, 12 minutes, 13 minutes, 14 minutes, or 15 minutes, wherein the thermal denaturation incubation is performed at a temperature of about 60° C. to about 100° C., such about any of 60° C., 65° C., 70° C., 75° C., 80° C., 85° C., 90° C., 91° C., 92° C., 93° C., 94° C., 95° C., 96° C., 97° C., 98° C., 99° C., or 100° C.

The thermal denaturation incubation temperature can be controlled by numerous techniques and combinations thereof. In some embodiments, the thermal denaturation incubation temperature is controlled by a water bath. In some embodiments, the thermal denaturation incubation temperature is controlled by a heat block. In some embodiments, the thermal denaturation incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the thermal denaturation incubation time temperature are controlled by a technique described herein, such as cooling at room temperature or via the thermocycler.

In some embodiments, the thermal denaturation technique comprises subjecting a sample, or a derivative thereof, e.g., a sample diluted in a buffer, to a thermal cycle. In some embodiments, the thermal cycle comprises subjecting the sample, or a derivative thereof, to one or more of: (a) a block starting temperature (b) block set temperature (the temperature for the thermal denaturation incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 60° C. to about 100° C., including about any of 60° C., 65° C., 70° C., 75° C., 80° C., 85° C., 90° C., 91° C., 92° C., 93° C., 94° C., 95° C., 96° C., 97° C., 98° C., 99° C., or 100° C. In some embodiments, the thermal cycle comprises: (a) a set block temperature of about 60° C. to about 100° C., including about any of 60° C., 65° C., 70° C., 75° C., 80° C., 85° C., 90° C., 91° C., 92° C., 93° C., 94° C., 95° C., 96° C., 97° C., 98° C., 99° C., or 100° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 50° C., such as any of 15° C. to about 25° C., about 20° C. to about 30° C., or about 20° C. to about 25° C., including about any of 20° C., 21° C., 22° C., 23° C., 24° C., 25° C., (b) a set block temperature of about 60° C. to about 100° C., such about any of 60° C., 65° C., 70° C., 75° C., 80° C., 85° C., 90° C., 91° C., 92° C., 93° C., 94° C., 95° C., 96° C., 97° C., 98° C., 99° C., or 100° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C. In some embodiments, the lid temperature during the thermal cycle may be the same respective temperature of the block during the thermal cycle or a temperature greater than the temperature of the block during the thermal cycle. In some embodiments, the lid temperature during the thermal cycle may be the same respective temperature of the block during the thermal cycle or a temperature greater than the temperature of the block during the thermal cycle.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

In some embodiments, the method further comprises admixing an amount of a blood sample, or portion thereof, a buffer prior to the thermal denaturation technique (e.g., the buffered sample is subjected to a thermal denaturation technique described herein). In some embodiment, the amount (as assessed based on the final concentration in the sample containing solution containing solution) of the buffer is about 1 mM to about 100 mM, such as any of about 20 mM to about 80 mM, about 30 mM to about 70 mM, or about 40 mM to about 60 mM. In some embodiment, the amount of the buffer is about any of 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some embodiments, the buffer is selected from the group consisting of ammonium bicarbonate, ammonium acetate, ammonium formate, triethylammonium bicarbonate, and Tris-HCl, or any combination thereof.

In some embodiments, the method further comprises determining the protein concentration in a blood sample or a derivative thereof.

### II. Reduction Techniques

In certain aspects, the methods provided herein comprise performing a reduction technique. In some embodiments, the reduction technique is performed on a sample, or a derivative thereof, following thermal denaturation. Reduction techniques, generally speaking, reduce (e.g., cleave) disulfide linkages between cysteine residues of one or more polypeptides to reduce the presence of polypeptide conformations that inhibit or prevent protease cleavage of the one or more polypeptides. Reduction techniques described herein comprise subjecting a sample, or a derivative thereof (e.g., a denatured sample), to an amount of a reducing agent and incubating for a reducing incubation time performed at a temperature or range thereof.

In some embodiments, the reducing agent is dithiothreitol (DTT), tris(2-carboxyethyl) phosphine (TCEP), beta-mercaptoethanol (BME), or a cysteine, or any mixture thereof.

In some embodiments, the amount (as assessed based on the final concentration in the sample containing solution containing solution) of a reducing agent, e.g., DTT, used in a reduction technique is about 1 mM to about 100 mM, such as any of about 1 mM to about 40 mM, about 1 mM to about 30 mM, about 5 mM to about 25 mM, about 10 mM, to about 20 mM, 20 mM to about 80 mM, about 30 mM to about 70 mM, or about 40 mM to about 60 mM. In some embodiments, the amount of reducing agent used in a reduction technique is at least about 1 mM, such as at least about any of 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some embodiments, the amount of reducing agent used in a reduction technique is about 100 mM or less, such as about any of 95 mM or less, 90 mM or less, 85 mM or less, 80 mM or less, 75 mM or less, 70 mM or less, 65 mM or less, 60 mM or less, 55 mM or less, 50 mM or less, 45 mM or less, 40 mM or less, 35 mM or less, 30 mM or less, 25 mM or less, 20 mM or less, 19 or less, 18 or less, 17 or less, 16 or less, 15 or less, 14 or less, 13 or less, 12 or less, 11 or less, 10 or less, 9 or less, 8 or less, 7 or less, 6 or less, 5 or less, 4 or less, 3 or less, 2 or less, or 1 or less. In some embodiments, the amount of reducing agent used in a reduction technique is about any of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In some embodiments, the reduction incubation time is about 10 minutes to about 120 minutes, such as any of about 30 minutes to about 60 minutes, about 40 minutes to about 60 minutes, about 45 minutes to about 55 minutes. In some embodiments, the reduction incubation time is at least about 20 minutes, such as at least about any of 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, or 115 minutes. In some embodiments, the reduction incubation time is about 120 minutes or less, such as about any of 115 minutes or less, 110 minutes or less, 105 minutes or less, 100 minutes or less, 95 minutes or less, 90 minutes or less, 85 minutes or less, 80 minutes or less, 75 minutes or less, 70 minutes or less, 65 minutes or less, 60 minutes or less, 55 minutes or less, 50 minutes or less, 45 minutes or less, 40 minutes or less, 35 minutes or less, 30 minutes or less, or 25 minutes or less. In some embodiments, the reduction incubation time is about any of 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, or 115 minutes.

In some embodiments, the reduction incubation time is performed at a temperature of about 20° C. to about 100° C., such as any of about 40° C. to about 80° C., about 50° C. to about 70° C., about 50° C. to about 60° C., about 55° C. to about 65° C., or about 60° C. to about 70° C. In some embodiments, the reduction incubation time is performed at a temperature of at least about 20° C., such as at least about any of 25° C., 30° C., 35° C., 40° C., 45° C., 50° C., 55° C., 60° C., 65° C., 70° C., 75° C., 80° C., 85° C., 90° C., or 95° C. In some embodiments, the reduction incubation time is performed at a temperature of about 95° C. or less, such as about any of 90° C. or less, 85° C. or less, 80° C. or less, 75° C. or less, 70° C. or less, 65° C. or less, 60° C. or less, 55° C. or less, 50° C. or less, 45° C. or less, 40° C. or less, 35° C. or less, 30° C. or less, or 25° C. or less. In some embodiments, the reduction incubation time is performed at a temperature of about any of 20° C., 25° C., 30° C., 35° C., 40° C., 45° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 75° C., 80° C., 85° C., 90° C., or 95° C. In some embodiments, the reduction incubation time is performed at a room temperature.

In some embodiments, the reduction technique comprises a reduction incubation time of about 30 minutes to about 70 minutes, such as about any of 35 minutes, 40 minutes, 45 minutes, 50 minutes, 55 minutes, 60 minutes, or 65 minutes, wherein the reduction incubation time is performed at a temperature of about 50° C. to about 70° C., such about any of 55° C., 60° C., or 65° C. In some embodiments, the reduction technique comprises use of an amount (as assessed based on the final concentration in the sample containing solution) of a reducing agent, e.g., DTT, of about 5 mM to about 25 mM, such as any of about 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, or 24 mM, and a reduction incubation time of about 30 minutes to about 70 minutes, such as about any of 35 minutes, 40 minutes, 45 minutes, 50 minutes, 55 minutes, 60 minutes, or 65 minutes, wherein the reduction incubation time is performed at a temperature of about 50° C. to about 70° C., such about any of 55° C., 60° C., or 65° C.

The reduction incubation temperature can be controlled by numerous techniques and combinations thereof. In some embodiments, the reduction incubation temperature is controlled by an ambient temperature, such as room temperature. In some embodiments, the reduction incubation temperature is controlled by a water bath. In some embodiments, the reduction incubation temperature is controlled by a heat block. In some embodiments, the reduction incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the reduction incubation time temperature are controlled by a technique described herein, such as cooling at room temperature or a ramp rate.

In some embodiments, the reduction technique comprises subjecting a sample, or a derivative thereof, e.g., a denatured sample, to a thermal cycle. In some embodiments, the thermal cycle comprises subjecting the sample, or a derivative thereof, to one or more of: (a) a block starting temperature (b) block set temperature (the temperature for the reduction incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 20° C. to about 100° C., such as any of 40° C. to about 80° C., about 50° C. to about 70° C., about 50° C. to about 60° C., about 55° C. to about 65° C., or about 60° C. to about 70° C., including about any of 50° C., 55° C., 60° C., 65° C., or 70° C. In some embodiments, the thermal cycle comprises: (a) a set block temperature of about 20° C. to about 100° C., such as any of about 40° C. to about 80° C., about 50° C. to about 70° C., about 50° C. to about 60° C., about 55° C. to about 65° C., or about 60° C. to about 70° C., including about any of 50° C., 55° C., 60° C., 65° C., or 70° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 60° C., such as any of about 15° C. to about 50° C., about 20° C. to about 40° C., about 20° C. to about 30° C., or about 20° C. to about 25° C., (b) a set block temperature of about 20° C. to about 100° C., such as any of about 40° C. to about 80° C., about 50° C. to about 70° C., about 50° C. to about 60° C., about 55° C. to about 65° C., or about 60° C. to about 70° C., including about any of 50° C., 55° C., 60° C., 65° C., or 70° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

In some embodiments, the reduction technique described herein is completed simultaneously with a thermal denaturation step. For example, the combined thermal denaturation technique and reduction technique comprises adding a reducing agent to a sample, or a derivative thereof, and then subjecting the sample, or the derivative thereof, to a temperature of about 60° C. to about 100° C., such about any of 60° C., 65° C., 70° C., 75° C., 80° C., 85° C., 90° C., 91° C., 92° C., 93° C., 94° C., 95° C., 96° C., 97° C., 98° C., 99° C., or 100° C., for an incubation time of at least about 1 minute, such as at least about any of 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes, or 60 minutes. In some embodiments, the combined thermal denaturation technique and reduction technique comprises adding a reducing agent to a sample, or a derivative thereof, and then subjecting the sample, or the derivative thereof, to a temperature of about 90° C. to about 100° C., for an incubation time of about 40 minutes to about 60 minutes, including 50 minutes.

### III. Alkylation Techniques

In certain aspects, the methods provided herein comprise performing an alkylation technique. In some embodiments, the alkylation technique is performed on a sample, or a derivative thereof, following the performance of a reduction technique. Alkylation techniques, generally speaking, prevent the reformation of one or more disulfide linkages between, e.g., cysteine residues of one or more polypeptides. This is done by, e.g., the addition of an acetamide moiety to the sulfur of a cysteine residue thereby producing an alkylated polypeptide. Alkylation techniques may reduce the presence of polypeptide conformations that inhibit or prevent protease cleavage of the one or more polypeptides. Alkylation techniques described herein comprise subjecting a sample, or a derivative thereof (e.g., a reduced sample), to an amount of an alkylating agent and incubating for an alkylation incubation time performed at a temperature or range thereof. In some embodiments, the method comprises subjecting a denatured sample to a reduction technique followed by an alkylation technique prior to performing a proteolytic digestion technique.

In some embodiments, the alkylating agent is iodoacetamide (IAA), 2-chloroacetamide, an acetamide salt, or any mixture thereof.

In some embodiments, the amount (as assessed based on the final concentration in the sample containing solution) of an alkylating agent, e.g., IAA, used in an alkylation technique is about 10 mM to about 100 mM, such as any of about 10 mM to about 50 mM, about 20 mM to about 40 mM, about 20 mM to about 36 mM, about 15 mM to about 25 mM, about 20 mM to about 25 mM, about 20 mM to about 80 mM, about 30 mM to about 70 mM, or about 40 mM to about 60 mM. In some embodiments, the amount of an alkylating agent used in an alkylation technique is at least about 10 mM, such as at least about any of 15 mM, 16 mM, 17 mM, 18 mM, 19 mM, 20 mM, 21 mM, 22 mM, 23 mM, 24 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some embodiments, the amount of an alkylating agent used in an alkylation technique is about 100 mM or less, such as about any of 95 mM or less, 90 mM or less, 85 mM or less, 80 mM or less, 75 mM or less, 70 mM or less, 65 mM or less, 60 mM or less, 55 mM or less, 50 mM or less, 45 mM or less, 40 mM or less, 35 mM or less, 30 mM or less, 25 mM or less, 24 mM or less, 23 mM or less, 22 mM or less, 21 mM or less, 20 mM or less, 19 mM or less, 18 mM or less, 17 mM or less, 16 mM or less, 15 mM or less, or 10 mM or less. In some embodiments, the amount of an alkylating agent used in an alkylation technique is about any of 10 mM, 15 mM, 20 mM, 20.5 mM, 21 mM, 21.5 mM, 22 mM, 22.5 mM, 23 mM, 23.5 mM, 24 mM, 24.5 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In some embodiments, the alkylation incubation time is about 5 minutes to about 60 minutes, such as any of about 10 minutes to about 50 minutes, about 20 minutes to about 40 minutes, or about 25 minutes to about 35 minutes. In some embodiments, the alkylation incubation time is at least about 5 minutes, such as at least about any of 10 minutes, 15 minutes, 20 minutes, 25 minutes, 30 minutes, 35 minutes, 40 minutes, 45 minutes, 50 minutes, 55 minutes, or 60 minutes. In some embodiments, the alkylation incubation time is about 60 minutes or less, such as about any of 55 minutes or less, 50 minutes or less, 45 minutes or less, 40 minutes or less, 35 minutes or less, 30 minutes or less, 25 minutes or less, 20 minutes or less, 15 minutes or less, 10 minutes or less, or 5 minutes or less. In some embodiments, the alkylation incubation time is about any of 5 minutes, 10 minutes, 15 minutes, 20 minutes, 25 minutes, 30 minutes, 35 minutes, 40 minutes, 45 minutes, 50 minutes, 55 minutes, or 60 minutes.

In some embodiments, the alkylation incubation time is performed at a temperature of about 15° C. to about 100° C., such as any of about 15° C. to about 80° C., about 15° C. to about 60° C., about 15° C. to about 35° C., about 20° C. to about 30° C., or about 20° C. to about 25° C. In some embodiments, the alkylation incubation time is performed at a temperature of at least about 15° C., such as at least about any of 20° C., 21° C., 22° C., 23° C., 24° C., 25° C., 30° C., 35° C., 40° C., 45° C., 50° C., 55° C., 60° C., 65° C., 70° C., 75° C., 80° C., 85° C., 90° C., or 95° C. In some embodiments, the alkylation incubation time is performed at a temperature of about 95° C. or less, such as about any of 90° C. or less, 85° C. or less, 80° C. or less, 75° C. or less, 70° C. or less, 65° C. or less, 60° C. or less, 55° C. or less, 50° C. or less, 45° C. or less, 40° C. or less, 35° C. or less, 30° C. or less, 25° C. or less, 24° C. or less, 23° C. or less, 22° C. or less, 21° C. or less, or 20° C. or less. In some embodiments, the alkylation incubation time is performed at a temperature of about any of 15° C., 16° C., 17° C., 18° C., 19° C., 20° C., 21° C., 22° C., 23° C., 24° C., 25° C., 30° C., 35° C., 40° C., 45° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 75° C., 80° C., 85° C., 90° C., or 95° C. In some embodiments, the alkylation incubation time is performed at a room temperature.

In some embodiments, the alkylation technique comprises an alkylation incubation time of about 5 minutes to about 60 minutes, such as about any of 15 minutes, 20 minutes, 25 minutes, 30 minutes, 35 minutes, or 40 minutes, wherein the alkylation incubation time is performed at a temperature of about 15° C. to about 30° C., such about any of 20° C., 21° C., 22° C., 23° C., 24° C., or 25° C. In some embodiments, the alkylation technique comprises use of an amount (containing solution based on the final concentration in the sample) of an alkylating agent, e.g., IAA, of about 15 mM to about 40 mM, such as any of about 20 mM, 20.5 mM, 21 mM, 21.5 mM, 22 mM, 22.5 mM, 23 mM, 23.5 mM, 24 mM, 24.5 mM, 25 mM, 25.5 mM, 26 mM, 26.5 mM, 27 mM, 27.5 mM, 28 mM, 28.5 mM, 29 mM, 29.5 mM, 30 mM, 30.5 mM, 31 mM, 31.5 mM, 32 mM, 32.5 mM, 33 mM, 33.5 mM, 34 mM, 34.5 mM, 35 mM, 35.5 mM, 36 mM, 36.5 mM, 37 mM, 37.5 mM, 38 mM, and an alkylation incubation time of about 5 minutes to about 60 minutes, such as about any of 15 minutes, 20 minutes, 25 minutes, 30 minutes, 35 minutes, or 40 minutes, wherein the alkylation incubation time is performed at a temperature of about 15° C. to about 30° C., such about any of 20° C., 21° C., 22° C., 23° C., 24° C., or 25° C.

The alkylation incubation temperature can be controlled by numerous techniques and combinations thereof. In some embodiments, the alkylation incubation temperature is controlled by an ambient temperature, such as room temperature. In some embodiments, the alkylation incubation temperature is controlled by a water bath. In some embodiments, the alkylation incubation temperature is controlled by a heat block. In some embodiments, the alkylation incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the alkylation incubation time temperature are controlled by a technique described herein, such as cooling at room temperature or a ramp rate.

In some embodiments, the alkylation technique comprises subjecting a sample, or a derivative thereof, e.g., a denatured sample, to a thermal cycle. In some embodiments, the thermal cycle comprises subjecting the sample, or a derivative thereof, to one or more of: (a) a block starting temperature (b) block set temperature (the temperature for the alkylation incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., or 25° C. In some embodiments, the thermal cycle comprises: (a) a set block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., or 25° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., or 25° C., (b) a set block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., or 25° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C.

In some embodiments, the alkylation technique further comprises quenching the alkylating agent comprising use of a neutralizing agent. In some embodiments, the neutralizing agent is a reducing agent. In some embodiments, the reducing agent is dithiothreitol (DTT), tris(2-carboxyethyl) phosphine (TCEP), beta-mercaptoethanol (BME), or a cysteine, or any mixture thereof. In some embodiments, the neutralizing agent is added in an amount to fully quench the amount of the alkylating agent, such as in an amount greater than or equal to a molar amount of an active moiety of the alkylating agent. In some embodiments, the amount (as assessed based on the final concentration in the sample containing solution) of the neutralizing agent is about 1 mM to about 100 mM.

In some embodiments, the alkylation technique, in whole or in part, is performed substantially in a low light condition. In some embodiments, the alkylation incubation time is performed in a low light condition. In some embodiments, the low light condition is in the dark or a location substantially devoid of sunlight and/or room lighting, such as in a desk drawer. In some embodiments, the low light condition is a filtered light, such as red light.

In some embodiments, the alkylating agent is sourced from a stock solution. In some embodiments, the stock solution is prepared within about 1 hour, such as within about any of 50 minutes, 40 minutes, 30 minutes, 20 minutes, or 10 minutes, of use.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

### IV. Proteolytic Digestion Techniques

In certain aspects, the methods provided herein comprise performing a proteolytic digestion technique. In some embodiments, the proteolytic digestion technique is performed on a sample, or a derivative thereof, following thermal denaturation and/or any additional steps intended to expose protease cleavage sites. Proteolytic digestion techniques, generally speaking, cleave polypeptides at known cleavage sites. For example, trypsin is a serine protease that generally cleaves polypeptides at the carboxyl side (C-terminal side) of lysine and arginine residues. Certain exceptions apply to the cleavage pattern of trypsin, such as due to proximity of a proline residue and/or a post-translational modification causing steric hindrance relative to the cleavage site. Proteolytic digestion techniques described herein comprise subjecting a sample, or a derivative thereof (e.g., a denatured sample or an alkylated sample, including an alkylated sample subjected to a reduction technique prior to an alkylation technique), to an amount of one or more proteases and incubating for a digestion incubation time performed at a temperature or range thereof.

In some embodiments, each of the one or more proteases is trypsin, LysC, LysN, AspN, GluC, ArgC, IdeS, IdeZ, PNGase F, thermolysin, pepsin, elastase, TEV, or Factor Xa, or any mixture thereof. In some embodiments, wherein two or more proteases are used, the weight ratio between a first protease and a second protease is about 1:10 to about 10:1, such as about any of about 1:9, 1:8, 1:7: 1:6, 1:5, 1:4, 1:3, 1:2, or 1:1. In some embodiments, the one or more proteases is trypsin. In some embodiments, the one or more proteases is a mixture of trypsin and LysC, such as in a weight ratio of about 1:1. In some embodiments, the one or more proteases is selected based on the type and/or characteristic of a blood sample, or components thereof, used in the methods herein. In some embodiments, the blood sample is processed by the absorbent or bibulous member such that a plasma sample is obtained, wherein the one or more proteases is trypsin and Lys-C, such as in a weight ratio of about 1:1. In some embodiments, the blood sample is processed by the absorbent or bibulous member such that a serum sample is obtained, wherein the one or more proteases is trypsin. In some embodiments, the protease is a modified protease, such as comprising a modification to prevent or inhibit self-proteolysis. In some embodiments, the modified protease is a modified trypsin, such as a methylated and/or an acetylated trypsin. In some embodiments, the modified trypsin is a tosyl phenylalanyl chloromethyl ketone (TPCK)-treated trypsin.

In some embodiments, the amount of a protease, e.g., trypsin or LysC, used in a proteolytic digestion technique is based on a weight ratio relative to the polypeptide content of a sample, or a derivative thereof, (i.e., weight of a protease: weight of polypeptide content) of about 1:200 to about 1:10, such as any of about 1:100 to about 1:10, about 1:50 to about 1:10, about 1:40 to about 1:20, about 1:50 to about 1:30, about 1:45 to about 1:35, about 1:20 to about 1:40, about 1:30 to about 1:10, or about 1:25 to about 1:15. In some embodiments, the amount of a protease used in a proteolytic digestion technique is at least about 1:200, such as at least about any of 1:190, 1:180, 1:170, 1:160, 1:150, 1:140, 1:130, 1:120, 1:110, 1:100, 1:95, 1:90, 1:85, 1:80, 1:75, 1:70, 1:65, 1:60, 1:55, 1:50, 1:45, 1:40, 1:35, 1:30, 1:25, 1:20, 1:15, or 1:10. In some embodiments, the amount of a protease used in a proteolytic digestion technique is about 1:10 or less, such as about any of 1:15 or less, 1:20 or less, 1:25 or less, 1:30 or less, 1:35 or less, 1:40 or less, 1:45 or less, 1:50 or less, 1:55 or less, 1:60 or less, 1:65 or less, 1:70 or less, 1:75 or less, 1:80 or less, 1:85 or less, 1:90 or less, 1:95 or less, 1:100 or less, 1:110 or less, 1:120 or less, 1:130 or less, 1:140 or less, 1:150 or less, 1:160 or less, 1:170 or less, 1:180 or less, 1:190 or less, or 1:200 or less. In some embodiments, the amount of a protease used in a proteolytic digestion technique is about any of 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, 1:110, 1:120, 1:130, 1:140, 1:150, 1:160, 1:170, 1:180, 1:190, or 1:200. In some embodiments, the proteolytic digestion technique comprises the use of two or more proteases, such as a combination of trypsin and LysC, and in such embodiments, the amount of each protease (such as described above) can be summed to a total amount of proteases used in a proteolytic digestion technique.

In some embodiments, the proteolytic digestion incubation time is about 20 minutes to about 36 hours, such as any of about 1 hour to about 18 hours, about 5 hours to about 24 hours, about 12 hours to about 24 hours, about 16 hours to about 20 hours, or about 12 hours to about 36 hours. In some embodiments, the proteolytic digestion incubation time is about 36 hours or less, such as about any of 32 hours or less, 30 hours or less, 28 hours or less, 26 hours or less, 24 hours or less, 22 hours or less, 20 hours or less, 19 hours or less, 18 hours or less, 17 hours or less, 16 hours or less, 15 hours or less, 14 hours or less, 13 hours or less, 12 hours or less, 11 hours or less, 10 hours or less, 9 hours or less, 8 hours or less, 7 hours or less, 6 hours or less, 5 hours or less, 4 hours or less, 3 hours or less, 2 hours or less, or 1 hour or less. In some embodiments, the proteolytic digestion incubation time is at least about 20 minutes, such as at least about any of 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 22 hours, 24 hours, 26 hours, 28 hours, 30 hours, 32 hours, 34 hours, or 36 hours. In some embodiments, the proteolytic digestion incubation time is about any of 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 22 hours, 24 hours, 26 hours, 28 hours, 30 hours, 32 hours, 34 hours, or 36 hours.

In some embodiments, the digestion incubation time is performed at a temperature of about 20° C. to about 60° C., such as any of about 20° C. to about 25° C., about 20° C. to about 30° C., about 25° C. to about 40° C., about 35° C. to about 40° C., or about 35° C. to about 50° C. In some embodiments, the digestion incubation time is performed at a temperature of at least about 20° C., such as at least about any of 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 42° C., 44° C., 46° C., 48° C., 50° C., 52° C., 54° C., 56° C., 58° C., or 60° C. In some embodiments, the digestion incubation time is performed at a temperature of about 60° C. or less, such about any of 58° C. or less, 56° C. or less, 54° C. or less, 52° C. or less, 50° C. or less, 48° C. or less, 46° C. or less, 44° C. or less, 42° C. or less, 40° C. or less, 39° C. or less, 38° C. or less, 37° C. or less, 36° C. or less, 35° C. or less, 34° C. or less, 33° C. or less, 32° C. or less, 31° C. or less, 30° C. or less, 29° C. or less, 28° C. or less, 27° C. or less, 26° C. or less, 25° C. or less, 24° C. or less, 23° C. or less, 22° C. or less, 21° C. or less, or 20° C. or less. In some embodiments, the digestion incubation time is performed at a temperature of about any of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 42° C., 44° C., 46° C., 48° C., 50° C., 52° C., 54° C., 56° C., 58° C., or 60° C. In some embodiments, the reduction incubation time is performed at a room temperature.

In some embodiments, the proteolytic digestion technique comprises a digestion incubation time of about 12 hours to about 24 hours, such as about any of 13 hours, 14 hours, 15 hours, 16 hours, 17 hours, 18 hours, 19 hours, 20 hours, 21 hours, 22 hours, 23 hours, wherein the digestion incubation time is performed at a temperature of about 20° C. to about 40° C., such about any of 22° C., 24° C., 26° C., 28° C., 30° C., 31° C., 32° C., 33° C., 34° C., 35° C., 36° C., 37° C., 38° C., 39° C. In some embodiments, the proteolytic digestion technique comprises use of an amount a protease for each of one or more proteases, e.g., trypsin and/or LysC, of about 1:15 to about 1:45, such as about any of 1:20, 1:25, 1:30, 1:35, or 1:40 (as measured based on the amount of the protease to the amount of polypeptide in a sample or a derivative thereof), and a digestion incubation time of about 12 hours to about 24 hours, such as about any of 13 hours, 14 hours, 15 hours, 16 hours, 17 hours, 18 hours, 19 hours, 20 hours, 21 hours, 22 hours, 23 hours, wherein the digestion incubation time is performed at a temperature of about 20° C. to about 40° C., such about any of 22° C., 24° C., 26° C., 28° C., 30° C., 31° C., 32° C., 33° C., 34° C., 35° C., 36° C., 37° C., 38° C., 39° C. In some embodiments, the proteolytic digestion technique is performed on a plasma sample or a derivate thereof, wherein the proteolytic digestion technique comprises a 1:40 ratio of trypsin to polypeptide in the sample of the derivative thereof and a 1:40 ratio of LysC to polypeptide in the sample or the derivative thereof. In some embodiments, the proteolytic digestion technique is performed on a serum sample or a derivate thereof, wherein the proteolytic digestion technique comprises a 1:20 ratio of trypsin.

The digestion incubation temperature can be controlled by numerous techniques and combinations thereof. In some embodiments, the digestion incubation temperature is controlled by an ambient temperature, such as room temperature. In some embodiments, the digestion incubation temperature is controlled by a water bath. In some embodiments, the digestion incubation temperature is controlled by a heat block. In some embodiments, the digestion incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the digestion incubation time temperature are controlled by a technique described herein, such as cooling at room temperature or a ramp rate.

In some embodiments, the proteolytic digestion technique comprises subjecting a sample, or a derivative thereof, e.g., an alkylated sample (including an alkylated sample quenched with a neutralizing agent), to a thermal cycle. In some embodiments, the thermal cycle comprises subjecting the sample, or a derivative thereof, to one or more of: (a) a block starting temperature (b) block set temperature (the temperature for the digestion incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 20° C. to about 50° C., including about any of 22° C., 24° C., 26° C., 28° C., 30° C., 31° C., 32° C., 33° C., 34° C., 35° C., 36° C., 37° C., 38° C., 39° C., 40° C., 42° C., 44° C., 46° C., or 48° C.

In some embodiments, the thermal cycle comprises: (a) a set block temperature of about 20° C. to about 50° C., including about any of 22° C., 24° C., 26° C., 28° C., 30° C., 31° C., 32° C., 33° C., 34° C., 35° C., 36° C., 37° C., 38° C., 39° C., 40° C., 42° C., 44° C., 46° C., or 48° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 35° C., such as any of about 20° C. to about 35° C., or about 20° C. to about 25° C., (b) a set block temperature of about 20° C. to about 50° C., including about any of 22° C., 24° C., 26° C., 28° C., 30° C., 31° C., 32° C., 33° C., 34° C., 35° C., 36° C., 37° C., 38° C., 39° C., 40° C., 42° C., 44° C., 46° C., or 48° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

In some embodiments, the proteolytic digestion technique further comprises quenching the one or more proteolytic enzymes. In some embodiments, quenching the one or more proteolytic enzymes comprises denaturing the one or more proteolytic enzymes. In some embodiments, quenching the one or more proteolytic enzymes comprise adding an amount of an acid. In some embodiments, the acid is formic acid (FA) or trifluoroacetic acid (TFA), or a mixture thereof. In some embodiments, the amount (as assessed based on the final concentration in the sample containing solution) of the acid added is about any of 0.1% v/v, 0.2% v/v, 0.3% v/v, 0.4% v/v, 0.5% v/v, 0.6% v/v, 0.7% v/v, 0.8% v/v, 0.9% v/v, 1% v/v, 1.1% v/v, 1.2% v/v, 1.3% v/v, 1.4% v/v, 1.5% v/v, 1.6% v/v, 1.7% v/v, 1.8% v/v, 1.9% v/v, or 2% v/v.

### V. Additional Techniques

In certain aspects, the method provided herein comprise subjecting the proteolytically digested sample comprising a proteolytic glycopeptide to one or more additional steps prior to subjecting the proteolytically digested sample, or a derivative thereof, to a liquid chromatography-mass spectrometry (LC-MS) technique using a liquid chromatography system and a mass spectrometer. In some embodiments, the LC system is online with the MS (i.e., eluate from the LC system is directly introduced to the MS). In some embodiments, the one or more additional steps do not include a desalting step performed outside of the LC system (such as an offline desalting technique).

In some embodiments, the method further comprises adding a standard to the proteolytically digested sample prior to the LC-MS technique. In some embodiments, the standard is a stable isotope-internal standard (SI-IS) peptide mixture.

In some embodiments, the biological sample is not subjected to a high-abundant protein depletion technique prior to the thermal denaturation technique. For example, in some embodiments, the high-abundant protein depletion technique removes highly abundant proteins present in a blood sample, such as serum albumin.

### E4. Methods for Performing a LC-MS Analysis of a Proteolytic Glycopeptide

In certain aspects, provided herein is a method for performing a LC-MS analysis on a sample comprising a proteolytic glycopeptide.

In some embodiments, the liquid chromatography (LC) system is online with a mass spectrometer (i.e., proteolytic peptide species, including glycopeptides, are eluted from the LC system directing into the mass spectrometer via a mass spectrometer interface. In some embodiments, the LC technique comprises performing a chromatographic separation of one or more proteolytic peptides, including glycopeptides. In some embodiments, the one or more proteolytic peptides subjected to a chromatographic separation are obtained from a proteolytically digested sample, such as described herein. In some embodiments, the chromatographic separation is performed on a proteolytically digested sample, such as described herein, (e.g., no additional separation technique, such as a sample clean-up step, is performed to remove one or more components from proteolytically digested sample). In some embodiments, the chromatographic separation is performed on a proteolytically digested sample comprising at least about 5 mM of a buffer, such as ammonium bicarbonate. In some embodiments, the chromatographic separation is performed on a proteolytically digested sample comprising an amount, such as at least about 1 mM, of a reducing agent or a byproduct thereof, such as a stable six-membered ring with an internal disulfide bond derived from DTT. In some embodiments, the chromatographic separation is performed on a proteolytically digested sample comprising an amount, such as at least about 1 mM, of an alkylating agent or a byproduct thereof, such as iodide (I-) derived from IAA.

In some embodiments, the method comprises introducing the proteolytically digested sample to a LC-MS system. In some embodiments, the method comprises performing a chromatographic separation of the proteolytically digested sample. In some embodiments, the chromatography separation comprises a period of diversion (i.e., diverted from the mass spectrometer interface, e.g., to a waste receptacle) of an initial eluate from the proteolytically digested sample. In some embodiments, the initial eluate (as assessed from the sample front) diverted from the mass spectrometer is about 1 column volume of the chromatographic column to about 5 column volumes of the chromatographic column, such as any of about 1 column volume to about 4 column volumes, about 2 column volumes to about 5 column volumes, or about 3 column volumes to about 4 column volumes. In some embodiments, the initial eluate (as assessed from the sample front) diverted from the mass spectrometer is at least about 0.5 column volumes, such as at least about any of 1 column volume, 1.5 column volumes, 2 column volumes, 2.5 column volumes, 3 column volumes, 3.5 column volumes, 4 column volumes, 4.5 column volumes, or 5 column volumes. In some embodiments, the initial eluate (as assessed from the sample front) diverted from the mass spectrometer is about 5 column volumes or less, such as about any of 4.5 column volumes or less, 4 column volumes or less, 3.5 column volumes or less, 3 column volumes or less, 2.5 column volumes or less, 2 column volumes or less, 1.5 column volumes or less, 1 column volume or less, or 0.5 column volume or less. In some embodiments, the initial eluate (as assessed from the sample front) diverted from the mass spectrometer is about any of 0.5 column volume, 1 column volume, 1.5 column volumes, 2 column volumes, 2.5 column volumes, 3 column volumes, 3.5 column volumes, 4 column volumes, 4.5 column volumes, or 5 column volumes.

In some embodiments, the proteolytically digest sample is subjected to a solid phase extraction column comprising a reversed phase material prior to subjecting the polypeptide content therein to a LC-MS analysis In some embodiments, the reversed-phase medium comprises an alkyl-based moiety, such as an alkyl-based moiety comprising an octadecyl carbon functional group (C18) covalently bound to a silica solid phase. In some embodiments, the silica solid phase comprises a plurality of particles having an average largest cross-sectional distance (such as a diameter, e.g., as measured by dynamic light scattering) of about 20 μm. In some embodiments, each particle of the plurality of particles comprises an average pore size of about 150 Å, and wherein the pores are derivitized with the alkyl-based moiety. In some embodiments, the solid phase extraction column comprising the reversed-phase medium has a column volume of about 5 μL. In some embodiments, the solid phase extraction column is an AssayMap 5 μL C18 cartridge (catalog no. 5190-6532; Agilent Technologies). In some embodiments, the reversed-phase medium comprises an underivitized polystyrene divinylbenzene hydrophobic reversed-phase resin. In some embodiments, the polystyrene divinylbenzene hydrophobic reversed-phase resin comprises a plurality of particles having an average largest cross-sectional distance (such as a diameter, e.g., as measured by dynamic light scattering) of about 20 μm. In some embodiments, each particle of the plurality of particles comprises an average pore size of about 100 Å. In some embodiments, the solid phase extraction column comprising the reversed-phase medium has a column volume of about 5 μL. In some embodiments, the solid phase extraction column is an AssayMAP 5 μL Reversed Phase (RP-S) cartridge (catalog no. G5496-60033; Agilent Technologies).

In some embodiments, the column volume refers to the volume occupied by the reversed-phase media within the sample phase extraction column or cartridge. The reversed-phase media can be referred to as a bed that is compacted within a chromatography column to form a bed volume.

In some embodiments, the chromatographic separation comprises a gradient separation performing using mixtures of an aqueous mobile phase and an organic mobile phase. In some embodiments, the chromatographic separation comprises isocratic period, such as a period of at least about 90%, such as at least about any of 91%, 92%, 93%, 94%, 95%, 96%, 97%, 98%, 99%, or 100%, of an aqueous mobile phase to produce the initial eluate that is diverted from the mass spectrometer interface.

In some embodiments, the LC system comprises a reversed-phase chromatography column. In some embodiments, the reversed-phase column comprises an alkyl moiety, such as C18.

The present application contemplates a diverse array of additional features of LC-MS techniques for analyzing a sample comprising a glycopeptide using a mass spectrometer. In some embodiments, the liquid chromatography system comprises a high performance liquid chromatography system. In some embodiments, the liquid chromatography system comprises an ultra-high performance liquid chromatography system. In some embodiments, the liquid chromatography system comprises a high-flow liquid chromatography system. In some embodiments, the liquid chromatography system comprises a low-flow liquid chromatography system, such as a micro-flow liquid chromatography system or a nano-flow liquid chromatography system. In some embodiments, the liquid chromatography system is coupled, such as directly interfaced, with a mass spectrometer.

In some embodiment, the mass spectrometry technique comprises an ionization technique. Ionization techniques contemplated by the present application include techniques capable of charging polypeptides and peptide products, including glycopeptides. Thus, in some embodiments, the ionization technique is electrospray ionization. In some embodiments, the ionization technique is nano-electrospray ionization. In some embodiments, the ionization technique is atmospheric pressure chemical ionization. In some embodiments, the ionization technique is atmospheric pressure photoionization.

A diverse array of mass spectrometers are contemplated as compatible with the description, include high-resolution mass spectrometers and low-resolution mass spectrometers. In some embodiments, the mass spectrometer is a time-of-flight (TOF) mass spectrometer. In some embodiments, the mass spectrometer is a quadrupole time-of-flight (Q-TOF) mass spectrometer. In some embodiments, the mass spectrometer is a quadrupole ion trap time-of-flight (QIT-TOF) mass spectrometer. In some embodiments, the mass spectrometer is an ion trap. In some embodiments, the mass spectrometer is a single quadrupole. In some embodiments, the mass spectrometer is a triple quadrupole (QQQ). In some embodiments, the mass spectrometer is an orbitrap. In some embodiments, the mass spectrometer is a quadrupole orbitrap. In some embodiments, the mass spectrometer is a Fourier transform ion cyclotron resonance (FT) mass spectrometer. In some embodiments, the mass spectrometer is a quadrupole Fourier transform ion cyclotron resonance (Q-FT) mass spectrometer. In some embodiments, the mass spectrometry technique comprises positive ion mode. In some embodiments, the mass spectrometry technique comprises negative ion mode. In some embodiments, the mass spectrometry technique comprises an ion mobility mass spectrometry technique.

In some embodiments, the LC-MS technique comprises processing obtained signals MS from the mass spectrometer. In some embodiments, the LC-MS technique comprises peak detection. In some embodiments, the LC-MS technique comprises determining ionization intensity of an ionized peptide product. In some embodiments, the LC-MS technique comprises determining peak height of an ionized peptide product. In some embodiments, the LC-MS technique comprises determining peak area of an ionized peptide product. In some embodiments, the LC-MS technique comprises determining peak volume of an ionized peptide product. In some embodiments, the LC-MS technique comprises identifying an ionized peptide product by amino acid sequence. In some embodiments, the LC-MS technique comprises determining the site of a post-translational modification of an ionized peptide, such as the site of a glycosylation. In some embodiments, the LC-MS technique comprises determining the glycan structure, or a characteristic thereof, of an ionized peptide product. In some embodiments, the LC-MS technique comprises manually validating the ionized peptide product acid sequence assignments. In some embodiments, the LC-MS technique comprises a quantification technique.

F4. Ovarian Cancer Biomarkers for Samples from Absorbent or Bibulous Members and Methods Thereof

In some embodiments, provided herein are methods for diagnosing a pelvic tumor (such as ovarian cancer) comprising detecting one or more biomarkers. In some embodiments, the one or more biomarkers comprise one or more glycopeptides. In some embodiments, the one or more biomarkers comprises one or more peptide structures set forth in Table 9. In some embodiments, the method comprises detecting one or more glycopeptides comprising a sequence set forth in SEQ ID NOs: 35-51. In some embodiments, the method comprises detecting one or more glycopeptides comprising a sequence set forth in SEQ ID NOs: 35-42. In some embodiments, the method comprises detecting one or more glycopeptides comprising a sequence set forth in SEQ ID NOs: 35-40. In some embodiments, the method comprises detecting one or more glycopeptides comprising a sequence set forth in SEQ ID NOs: 43-51. In some embodiments, the glycopeptide comprises a glycan with the structure 6513 in Table 10. In some embodiments, the diagnosis is a determination of whether the individual has a benign or malignant tumor. In some embodiments, the tumor is ovarian cancer.

In some embodiments, the diagnosis is based upon presence and/or amount of at least one, at least two, at least three, at least four, at least five, at least six, at least seven or eight peptide structures from Table 9. In some embodiments, the diagnosis is based upon the presence and/or amount of one or more peptides comprising the amino acid sequence of SEQ ID NOs: 35-42. In some embodiments, the diagnosis is based upon the presence and/or amount of two or more peptides comprising the amino acid sequence of SEQ ID NOs: 35-42. In some embodiments, the diagnosis is based upon the presence and/or amount of three or more peptides comprising the amino acid sequence of SEQ ID NOs: 35-42. In some embodiments, the diagnosis is based upon the presence and/or amount of four or more peptides comprising the amino acid sequence of SEQ ID NOs: 35-42. In some embodiments, the diagnosis is based upon the presence and/or amount of five or more peptides comprising the amino acid sequence of SEQ ID NOs: 35-42. In some embodiments, the diagnosis is based upon the presence and/or amount of six or more peptides comprising the amino acid sequence of SEQ ID NOs: 35-42. In some embodiments, the diagnosis is based upon the presence and/or amount of seven or more peptides comprising the amino acid sequence of SEQ ID NOs: 35-42. In some embodiments, the diagnosis is based upon the presence and/or amount of each of the peptides comprising the amino acid sequence of SEQ ID NOs: 35-42.

In some embodiments, the diagnosis is based upon the presence and/or amount of one or more peptides consisting of the amino acid sequence of SEQ ID NOs: 35-42. In some embodiments, the diagnosis is based upon the presence and/or amount of two or more peptides consisting of the amino acid sequence of SEQ ID NOs: 35-42. In some embodiments, the diagnosis is based upon the presence and/or amount of three or more peptides consisting of the amino acid sequence of SEQ ID NOs: 35-42. In some embodiments, the diagnosis is based upon the presence and/or amount of four or more peptides consisting of the amino acid sequence of SEQ ID NOs: 35-42. In some embodiments, the diagnosis is based upon the presence and/or amount of five or more peptides consisting of the amino acid sequence of SEQ ID NOs: 35-42. In some embodiments, the diagnosis is based upon the presence and/or amount of six or more peptides consisting of the amino acid sequence of SEQ ID NOs: 35-42. In some embodiments, the diagnosis is based upon the presence and/or amount of seven or more peptides consisting of the amino acid sequence of SEQ ID NOs: 35-42. In some embodiments, the diagnosis is based upon the presence and/or amount of each of the peptides consisting of the amino acid sequence of SEQ ID NOs: 35-42.

In some embodiments, the diagnosis is based upon the presence and/or amount of one or more peptides comprising the amino acid sequence of SEQ ID NOs: 43-51. In some embodiments, the diagnosis is based upon the presence and/or amount of two or more peptides comprising the amino acid sequence of SEQ ID NOs: 43-51. In some embodiments, the diagnosis is based upon the presence and/or amount of three or more peptides comprising the amino acid sequence of SEQ ID NOs: 43-51. In some embodiments, the diagnosis is based upon the presence and/or amount of four or more peptides comprising the amino acid sequence of SEQ ID NOs: 43-51. In some embodiments, the diagnosis is based upon the presence and/or amount of five or more peptides comprising the amino acid sequence of SEQ ID NOs: 43-51. In some embodiments, the diagnosis is based upon the presence and/or amount of six or more peptides comprising the amino acid sequence of SEQ ID NOs: 43-51. In some embodiments, the diagnosis is based upon the presence and/or amount of seven or more peptides comprising the amino acid sequence of SEQ ID NOs: 43-51. In some embodiments, the diagnosis is based upon the presence and/or amount of each of the peptides comprising the amino acid sequence of SEQ ID NOs: 43-51.

In some embodiments, the diagnosis is based upon the presence and/or amount of one or more peptides consisting of the amino acid sequence of SEQ ID NOs: 43-51. In some embodiments, the diagnosis is based upon the presence and/or amount of two or more peptides consisting of the amino acid sequence of SEQ ID NOs: 43-51. In some embodiments, the diagnosis is based upon the presence and/or amount of three or more peptides consisting of the amino acid sequence of SEQ ID NOs: 43-51. In some embodiments, the diagnosis is based upon the presence and/or amount of four or more peptides consisting of the amino acid sequence of SEQ ID NOs: 43-51. In some embodiments, the diagnosis is based upon the presence and/or amount of five or more peptides consisting of the amino acid sequence of SEQ ID NOs: 43-51. In some embodiments, the diagnosis is based upon the presence and/or amount of six or more peptides consisting of the amino acid sequence of SEQ ID NOs: 43-51. In some embodiments, the diagnosis is based upon the presence and/or amount of seven or more peptides consisting of the amino acid sequence of SEQ ID NOs: 43-51. In some embodiments, the diagnosis is based upon the presence and/or amount of each of the peptides consisting of the amino acid sequence of SEQ ID NOs: 43-51.

In some embodiments, provided herein is a method of treating ovarian cancer in an individual based upon the presence, absence, or amount of one or more peptide structures set forth in Table 9. In some embodiments, one or more peptide structures set forth in SEQ ID NOs: 35-42 is detected. In some embodiments, one or more peptide structures set forth in SEQ ID NOs: 35-40 is detected. In some embodiments, one or more peptide structures set forth in SEQ ID NOs: 43-51 is detected. In some embodiments, the method further comprises delivering a therapeutic agent based upon the presence, absence, or amount of one or more peptide structures set forth in Table 9. In some embodiments, the method comprises selecting a therapeutic agent based upon the presence, absence, or amount of one or more peptide structures set forth in Table 9. In some embodiments, the therapeutic agent is a chemotherapeutic agent and/or a hormone therapy.

### G4. Exemplary Methods

In some embodiments, provided herein is a method of classifying a biological sample obtained from a subject with respect to a plurality of states associated with a pelvic cancer or a method of determining a state associated with a pelvic cancer comprising detecting one or more peptide structures set forth in Table 9, such as SEQ ID NOs: 35-42, SEQ ID NOs: 35-40 or SEQ ID NOs: 43-51. In some embodiments, the sample is derived from blood. In some embodiments, the sample is from an absorbent or bibulous member, such as a dried blood spot card. In some embodiments, the absorbent or bibulous member comprises one or more polypeptide standards described herein. In some embodiments, the absorbent or bibulous member comprised one or more polypeptide standards prior to deposition of the blood. In some embodiments, the method further comprises processing a portion of the dried blood spot for mass spectrometry. In some embodiments, the method comprises extracting polypeptides from a portion the absorbent or bibulous member. In some embodiments, the method comprises proteolytic processing extracted polypeptides. In some embodiments, the method comprises digestion with a protease. In some embodiments, the method comprises digestion with trypsin. In some embodiments, the absorbent or bibulous member is filter paper or cellulose. In some embodiments, the absorbent or bibulous member is a material that prevents hemolysis.

In some embodiments, the method further comprises performing mass spectrometry on the sample to detect one or more peptide structures set forth in Table 9, such as SEQ ID NOs: 35-42, SEQ ID NOs: 35-40, or SEQ ID NOs: 43-51. In some embodiments, the mass spectrometry is LC-MS. In some embodiments, the mass spectrometry is MRM-MS. In some embodiments, one or more parameters set forth in Table 11 is detected.

### H4. Samples and Components Thereof

The methods provided herein are contemplated to be suitable for analyzing a diverse array of samples, such as any blood samples or derivatives thereof obtained using an absorbent or bibulous member, such as a blood spot card. In some embodiments, the sample is a blood sample, such as a whole blood sample. In some embodiments, the blood sample is processed, such as by the absorbent or bibulous member, e.g., a lateral flow dried blood collection device. In some embodiments, the blood sample is a plasma sample. In some embodiments, the blood sample is a serum sample. In some embodiments, the sample is a tumor tissue. In some embodiments, the sample is from a subject suspected of having a pelvic tumor. In some embodiments, the sample is from a subject suspected of having ovarian cancer.

The methods provided herein are particularly useful for the analysis of blood samples, or portion thereof, comprising a glycoprotein, such as to generate glycopeptide containing specimens for analysis with a mass spectrometer.

In some embodiments, the method comprises an upstream sample preparation technique, such as for obtaining plasma or serum from a blood sample, performed prior to methods for proteolytically digesting a sample. In some embodiments, the upstream sample preparation technique comprises a cell lysis step. In some embodiments, the upstream sample preparation technique comprises a filtration step. In some embodiments, the upstream sample preparation technique comprises a dilution step. In some embodiments, the upstream sample preparation technique comprises a protein concentration determination step.

In some embodiments, the sample is obtained from an individual. In some embodiments, the sample is obtained from a human individual.

### I4. Systems, Kits, and Compositions

In certain aspects, contemplated herein are systems, kits, and compositions useful for performing the methods described herein. In some embodiments, provided herein is a system, kit, and/or composition useful for performing a proteolytic digestion of a blood sample, or a portion thereof, comprising a glycoprotein as described herein. In some embodiments, provided herein is a system, kit, and/or composition useful for performing a LC-MS analysis of a proteolytic glycopeptide as described herein. In some embodiments, provided herein is a system, kit, and/or composition useful for performing a proteolytic digestion of a blood sample, or a portion thereof, comprising a glycoprotein followed by LC-MS analysis of the proteolytic glycopeptide produced therefrom.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### Section 5—HILIC Enrichment Sample Preparation for Quantitative Mass Spectrometry

Provided herein, in certain aspects, are methods for processing a proteolytic digest sample for use in a liquid chromatography-mass spectrometry (LC-MS) analysis, the methods comprising loading a hydrophilic interaction liquid chromatography (HILIC) load derived from the proteolytic digest sample to a solid phase extraction column comprising a HILIC medium according to one or more conditions to associate the at least one proteolytically digested glycopeptide with the HILIC medium. The disclosure of the present application is based on the inventors' unique perspective and unexpected findings regarding methods for processing a proteolytic digest sample that provide an improved LC-MS analysis of glycopeptides, the methods comprising loading a HILIC load according to one or more of the following conditions: (1) the loading of the HILIC load to the solid phase extraction column is initiated when the HILIC medium is in a dry state; (2) the HILIC load loaded to the solid phase extraction column has an amount of the plurality of proteolytically digested peptides characterized by one or both of: (a) a ratio of the weight of the plurality of proteolytically digested peptides over the weight of the HILIC medium in the dry state of at least about 0.05, including at least about 0.06; and/or (b) a ratio of the weight of the plurality of proteolytically digested peptides relative to the bed volume of the HILIC medium in the dry state of at least about 30 μg/μl, including at least about 40 μg/μl; or (3) the HILIC load loaded to the solid phase extraction column has a concentration of an organic solvent of at least about 70% (v/v). The methods taught herein were demonstrated to enrich glycopeptide species derived from a proteolytic digest sample such that results from LC-MS analysis were significantly improved as compared to conventional glycopeptide LC-MS analyses. As demonstrated herein, in certain aspects the processing methods taught herein enable accurate and robust glycopeptide quantification. The results provided herein demonstrate high-throughput HILIC enrichment methods that allowed selective identification and reproducible quantification of glycopeptides from complex biologic matrices such as serum/plasma, and represent significant advancements in the ability to use glycoproteins in the study of human physiology, such as for disease diagnosis and treatment monitoring.

Thus, in some aspects, provided herein is a method for a method for processing a proteolytic digest sample for use in a LC-MS analysis, wherein the proteolytic digest sample comprises a plurality of proteolytically digested peptides comprising at least one proteolytically digested glycopeptide, the method comprising: (A) loading a HILIC load derived from the proteolytic digest sample to a solid phase extraction column comprising a HILIC medium according to one or more conditions to associate the at least one proteolytically digested glycopeptide with the HILIC medium, the one or more conditions comprising: (1) the loading of the HILIC load to the solid phase extraction column is initiated when the HILIC medium is in a dry state; (2) the HILIC load loaded to the solid phase extraction column has an amount of the plurality of proteolytically digested peptides characterized by one or both of: (a) a ratio of the weight of the plurality of proteolytically digested peptides over the weight of the HILIC medium in the dry state of at least about 0.05, including at least about 0.06; and/or (b) a ratio of the weight of the plurality of proteolytically digested peptides relative to the bed volume of the HILIC medium in the dry state of at least about 30 μg/μl, including at least about 40 μg/μl; or (3) the HILIC load loaded to the solid phase extraction column has a concentration of an organic solvent of at least about 70% (v/v); and (B) subjecting the HILIC medium to an elution solution to obtain a HILIC eluate comprising the at least one proteolytically digested glycopeptide.

### B5. Example Mass Spectrometry and Sample Preparation Workflow

For purposes of orientation and illustration of the description herein, provided in this section are example aspects of sample preparation and mass spectrometry workflows (FIGS. 1A-1C) for analyzing the composition of a peptide and/or glycopeptide using a mass spectrometer. Subsequent sections are provided with more details regarding certain inventive features related to methods for processing a proteolytic digest sample derived from a biological sample comprising a glycoprotein, methods of performing a LC-MS analysis of a proteolytic glycopeptide, and mass spectrometry workflows involving any combination of elements thereof.

FIG. 1A is a schematic of an example workflow 100 for a peptide structure analysis, including of glycopeptides. The workflow 100 may include various operations including, for example, sample collection 102, sample intake 104, sample preparation and mass spectrometry processing 106, and data analysis 108.

Sample collection 102 may include, for example, obtaining a biological sample 112 from an individual 114. A biological sample 112 may take the form of a specimen obtained via one or more sampling methods. A biological sample 112 may be representative of an individual 114 as a whole or of a specific tissue, cell type, or other category or sub-category of interest. In some embodiments, the biological sample 112 includes a whole blood sample 116 obtained via a blood draw. In some embodiments, the biological sample 112 includes set of aliquoted samples 118 that include, for example, a serum sample, a plasma sample, a blood cell (e.g., white blood cell (WBC), red blood cell (RBC) sample, another type of sample, or a combination thereof. In some embodiments, the biological sample 112 is a plasma sample from the individual 114. In some embodiments, the biological sample 112 is a serum sample from the individual 114. In some embodiments, the biological sample 112 may include nucleotides (e.g., ssDNA, dsDNA, RNA), organelles, amino acids, peptides, proteins, carbohydrates, glycoproteins, or any combination thereof.

Sample intake 104 may include one or more various operations such as, for example, aliquoting, labeling, registering, processing, storing, thawing, and/or other types of operations involved with preparing a sample for sample preparation and mass spectrometry processing.

Sample preparation and mass spectrometry processing 106 may include, for example, one or more operations to form set of peptide species 122, such as a proteolytically digested peptide and/or a proteolytically digested glycopeptide. In some embodiments, the sample preparation includes subjecting a biological sample to a proteolytic digestion. Mass spectrometry processing 124 may include, for example, liquid chromatography, introducing species from the sample, and/or derived therefrom, to a mass spectrometer, and data acquisition, such as using a multiple reaction monitoring (MRM) technique. MRM is a mass spectrometry method in which a precursor ion of a particular m/z value, including window thereof, (e.g., peptide analyte) is selected in the first quadrupole (Q1) and transmitted to the second quadrupole (Q2) for fragmentation. The resulting product ions are then transmitted to the third quadrupole (Q3), which detects only product ions with selected predefined m/z values. In some embodiments, the predefined m/z value, including window thereof, selected in the first quadrupole and a predefined m/z value, including window thereof, may be expressed as a MRM transition. Dynamic MRM (dMRM) is a variant of MRM. In dynamic MRM mode, MRM transition lists are scheduled throughout an LC/MS run based on the retention time window for each analyte. In this way, analytes are only monitored while they are eluting from the LC and therefore the MS scan time is not wasted by monitoring the analytes when they are not expected.

Data analysis 108 may include, for example, peptide structure analysis 126, e.g., determining the amino acid sequence of a peptide, determining a site of a post-translational modification, and/or determining a glycan composition and/or structure. In some embodiments, data analysis 108 also includes output generation 110. In some embodiments, output generation 110 may be considered a separate operation from data analysis 108. Output generation 110 may include, for example, generating final output 128 based on the results of peptide structure analysis 126. In some embodiments, the final output 128 may be used for one or more downstream purposes, such as research, diagnosis, and/or treatment, and may be sent to a remote system 130.

In certain aspects, the workflow 100 may optionally exclude one or more of the operations described herein and/or may optionally include one or more other steps or operations other than those described herein (e.g., in addition to and/or instead of those described herein).

FIG. 1B is a schematic of an example workflow 200 for certain sample preparation techniques 106, some of which may be optionally used in methods provided herein, including proteolytic digestion techniques to produce a proteolytic digest sample. In some embodiments, the workflow 200 comprises a denaturation step 202, such as to unfold and/or linearize a polypeptide to expose one or more cleavage sites. In some embodiments, the workflow 200 comprises a reduction step 202, such as to cleave disulfide bonds. In some embodiments, the workflow 200 comprises an alkylation technique 204, such as to modify cysteine residues to prevent reformation of a disulfide bond. In some embodiments, the workflow 200 comprises a proteolytic digestion technique 206, such as to produce proteolytically digested peptides, including proteolytically digested glycopeptides. Box 205 can represent the R group of an amino acid such as, for example, an R group of arginine or lysine that typically will direct a tryptic cleavage. In some embodiments, the workflow 200 may comprise a post-digestion procedure 207, such as any one or more of a desalting technique, addition of a standard, aliquoting, and/or preparation for a mass spectrometry analysis.

FIG. 1C is a schematic of an example workflow for certain mass spectrometry processing techniques 106, some of which may be optionally used in methods provided herein. In some embodiments, the workflow comprises a quantification technique 208 using a mass spectrometer, such as a liquid chromatography-mass spectrometry system. In some embodiments, the workflow comprises a quality control technique 210 configured to optimize data quality. In some embodiments, measures can be put in place allowing only errors within acceptable ranges outside of an expected value. In some embodiments, employing statistical models (e.g., using Westgard rules) can assist in quality control 210. For example, quality control 210 may include, for example, assessing the retention time and abundance of representative peptide species (e.g., glycosylated and/or aglycosylated peptide species) and spiked-in internal standards, in either every sample, or in each quality control sample (e.g., pooled serum digest). In some embodiments, the workflow comprises a peak integration and normalization technique 212 to process the data that has been generated and transform the data into a format for analysis. For example, peak integration and normalization 212 may include converting abundance data for various productions that were detected for a selected peptide species into a single quantification metric (e.g., a relative quantity, an adjusted quantity, a normalized quantity, a relative concentration, an adjusted concentration, or a normalized concentration) for that peptide structure. In some embodiments, peak integration and normalization 212 may be performed using one or more of the techniques described in U.S. Patent Publication No. 2020/0372973A1 and/or US Patent Publication No. 2020/0240996A1, the disclosures of which are incorporated by reference herein in their entireties.

### C5. Methods for Processing a Proteolytic Digest Sample

Provided herein, in certain aspects, are methods of processing a proteolytic digest sample using a solid phase extraction column comprising a comprising a hydrophilic interaction liquid chromatography (HILIC) medium. As taught herein, the described methodology may be used for the enrichment of glycopolypeptides (e.g., a proteolytically digested glycopeptide), from a sample such as a proteolytic digest sample. In some embodiments, the resulting composition obtained from the methodology provided herein, or a derivative thereof, is suitable for use in a liquid chromatography-mass spectrometry (LC-MS) analysis, such as for the analysis of glycopolypeptides. In some embodiments, the enriched glycopeptide is a proteolytically digested glycopeptide. In some embodiments, the enriched glycopeptide is an endogenous glycopeptide. In some embodiments, the glycopeptide obtained from the method provided herein is enriched by a factor of at least 30 when compared to the polypeptide content in a respective proteolytic digest sample.

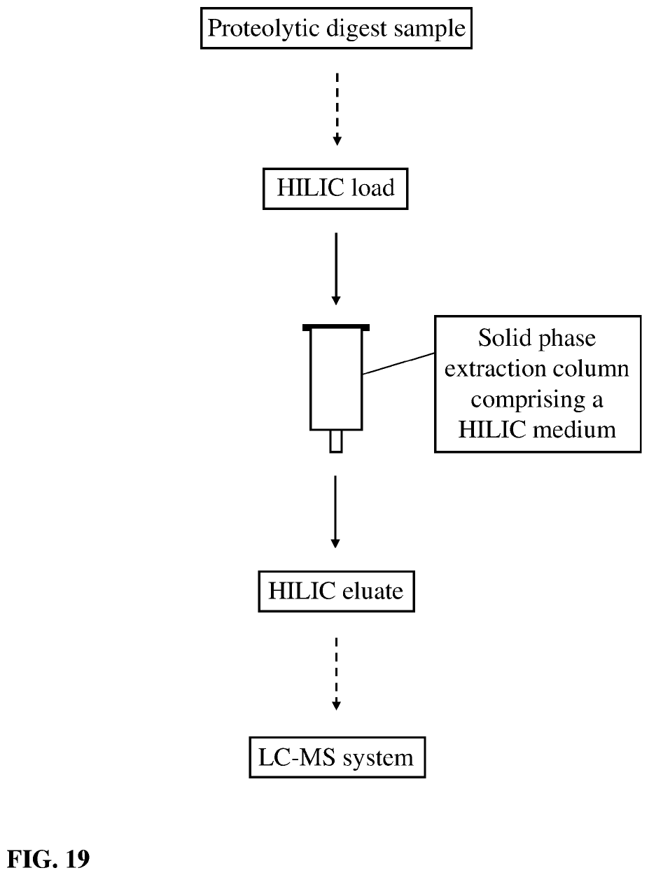

Aspects of the disclosure provided herein may be described via the state of the HILIC medium (e.g., dry state) when initiating loading of a HILIC load to a solid phase extraction column comprising a HILIC medium, and/or one or more conditions of the HILIC load, such as an amount of a plurality of proteolytically digested peptides relative to an amount of the HILIC medium in the solid phase extraction column and/or a concentration of an organic solvent of the HILIC load. In some embodiments, methodology is exemplified for obtaining the HILIC load having the one or more conditions. For purposes of orientation of aspects of the methodology provided herein, FIG. 19 illustrates an example workflow from a proteolytic digest sample to a LC-MS system. In some embodiments, the proteolytic digest sample resulting from a proteolytic digestion is not suitable for direct introduction to the LC-MS system (or it is not desirable to do so), such as due to conditions needed to facilitate reactions relevant to the proteolytic digestion (e.g., high salt and/or buffer content). As provided herein, in some embodiments, components of the proteolytic digest sample, such as a plurality of proteolytically digested peptides comprising at least one proteolytically digested glycopeptide, are subjected to a solid phase extraction column comprising a HILIC medium to associate the at least one proteolytically digested glycopeptide with the HILIC medium. The composition comprising the plurality of proteolytically digested peptides directly loaded onto the solid phase extraction column is referred to herein as a HILIC load (represented by the solid arrow in FIG. 19). In some embodiments, the methodology provided herein comprises loading a HILIC load having a specified condition (such as polypeptide amount relative to the amount of HILIC medium and/or concentration of an organic solvent) to a solid phase extraction column comprising a HILIC medium. In some embodiments, the proteolytic digest sample is subjected to one or more processing steps to form the HILIC load (represented by the dashed arrow in FIG. 19). As shown in FIG. 19, a HILIC eluate comprising the at least one glycopeptide is obtained from the solid phase extraction column. In some embodiments, the HILIC eluate is subjected to one or more processing steps in preparation for subjecting the at least one glycopeptide to the LC-MS system (represented by the dashed arrow in FIG. 19).

As described in certain aspects the instant application, in some embodiments, provided is a method for processing a proteolytic digest sample for use in a LC-MS analysis, wherein the proteolytic digest sample comprises a plurality of proteolytically digested peptides comprising at least one proteolytically digested glycopeptide, the method comprising loading a HILIC load derived from the proteolytic digest sample to a solid phase extraction column comprising a HILIC medium to associate the at least one proteolytically digested glycopeptide with the HILIC medium, wherein the loading of the HILIC load to the solid phase extraction column is initiated when the HILIC medium is in a dry state. As described in certain aspects the instant application, in some embodiments, provided is a method for processing a proteolytic digest sample for use in a LC-MS analysis, wherein the proteolytic digest sample comprises a plurality of proteolytically digested peptides comprising at least one proteolytically digested glycopeptide, the method comprising loading a HILIC load derived from the proteolytic digest sample to a solid phase extraction column comprising a HILIC medium to associate the at least one proteolytically digested glycopeptide with the HILIC medium, wherein the HILIC load loaded to the solid phase extraction column has an amount of the plurality of proteolytically digested peptides characterized by one or both of: (a) a ratio of the weight of the plurality of proteolytically digested peptides over the weight of the HILIC medium in the dry state of at least about 0.06; and/or (b) a ratio of the weight of the plurality of proteolytically digested peptides relative to the bed volume of the HILIC medium in the dry state of at least about 40 μg/μl. As described in certain aspects the instant application, in some embodiments, provided is a method for processing a proteolytic digest sample for use in a LC-MS analysis, wherein the proteolytic digest sample comprises a plurality of proteolytically digested peptides comprising at least one proteolytically digested glycopeptide, the method comprising loading a HILIC load derived from the proteolytic digest sample to a solid phase extraction column comprising a HILIC medium to associate the at least one proteolytically digested glycopeptide with the HILIC medium, wherein the HILIC load loaded to the solid phase extraction column has a concentration of an organic solvent of at least about 70% (v/v). As described in certain aspects the instant application, in some embodiments, provided is a method for processing a proteolytic digest sample for use in a LC-MS analysis, the method comprising any one or more combinations of the steps described herein.

In the following sections, additional description of the various aspects of the methods for processing a proteolytic digest sample is provided. Such description in a modular fashion is not intended to limit the scope of the disclosure, and based on the teachings provided herein one of ordinary skill in the art will readily appreciate that certain modules can be integrated, at least in part. The section heading used herein are for organizational purposes only and are not to be construed as limiting the subject matter described.

### I. Dry State of HILIC Medium and HILIC Medium Characteristics

Provided herein, in some aspects, is a method for processing a proteolytic digest sample for use in a LC-MS analysis, the method comprising loading a HILIC load to a solid phase extraction column comprising a HILIC medium, wherein the loading of the HILIC load to the solid phase extraction column is initiated when the HILIC medium is in a dry state. One of ordinary skill in the art will readily appreciate that a HILIC medium will not remain in a dry state over the entire course of loading a HILIC load, i.e., the HILIC load will wet the HILIC medium over the course of applying the HILIC load to the HILIC medium. In some embodiments, the description of a dry state of a HILIC medium references the state of the HILIC medium immediately prior to a first portion of a HILIC load contacting the HILIC medium.

In some embodiments, the dry state of a HILIC medium is characterized by the HILIC medium comprising less than about 5% (v/v), such as less than about any of 4.5% (v/v), 4% (v/v), 3.5% (v/v), 3% (v/v), 2.5% (v/v), 2% (v/v), 1.5% (v/v), 1% (v/v), 0.5% (v/v), or 0.1% (v/v), of a liquid at the initiation of the loading of a HILIC load to a solid phase extraction column comprising the HILIC medium. In some embodiments, the HILIC medium is substantially dry, such as does contain a liquid in a measurable amount, such as above the level of liquid in surrounding air, e.g., as based on humidity. In some embodiments, the liquid comprises water.

In some embodiments, at the initiation of the loading of a HILIC load to a HILIC medium, the HILIC medium has not been subjected to a washing, equilibration, or wetting procedure. It is appreciated that in some embodiments, the HILIC medium may have been subjected to a liquid such as during manufacturing or packaging, and in certain aspects the present description regarding a dry state is focused on steps that occur after the HILIC medium is loaded into a cartridge to form the solid phase extraction column and dried (or a occur after a final drying step and prior to loading a HILIC load). For example, in some embodiments, the method comprises obtaining a solid phase extraction column comprising a HILIC medium, such as from a manufacturer, and loading a HILIC load to the solid phase extraction column without intervening steps of introducing a liquid to the solid phase extraction column, such as for purposes of a washing, equilibration, or wetting procedure. In some embodiments, at the initiation of the loading of a HILIC load to a HILIC medium, the HILIC medium is not in an equilibrated state. In some embodiments, at the initiation of the loading of a HILIC load to a HILIC medium, the HILIC medium is not in a wetted state. In some embodiments, at the initiation of the loading of a HILIC load to a HILIC medium, the HILIC medium is not in a swelled state. In some embodiments, at the initiation of the loading of a HILIC load to a HILIC medium, the HILIC medium is not in a hydrated state. In some embodiments, the HILIC medium may be subjected to a liquid, such as a washing liquid, and then subsequently dried, wherein at the initiation of the loading of a HILIC load to the HILIC medium, the HILIC load is in a dry state.

In some embodiments, at the initiation of the loading of a HILIC load on a HILIC medium, the HILIC medium is not equilibrated with an equilibration liquid. In some embodiments, at the initiation of the loading of a HILIC load on a HILIC medium, the HILIC medium is not washed with a wash liquid. In some embodiments, at the initiation of the loading of a HILIC load on a HILIC medium, the HILIC medium is not wetted with a wetting liquid. In some embodiments, during the loading of the HILIC load, the HILIC medium is hydrated with water and then binds to at least one glycopeptide, wherein the HILIC load includes both water and an organic miscible solvent.

In some embodiments, the method provided herein comprises drying a HILIC medium of a solid phase extraction column to obtain the HILIC medium in a dry state. In some embodiments, the drying comprises subjecting a solid phase extraction column, or a HILIC medium, to conditions such that a liquid in the solid phase extraction column of the HILIC medium can leave the solid phase extraction column. In some embodiments, the condition comprises a room temperature. In some embodiments, the condition comprises an elevate temperature. In some embodiments, the method comprises determining the liquid content of a HILIC medium to assess if it is in a dry state prior to loading a HILIC load to a solid phase extraction column comprising the HILIC medium. In some embodiments, drying a HILIC medium of a solid phase extraction column may comprises use of a force to push liquid through the solid phase extraction column, such as a centrifugal force, position pressure, and/or negative pressure.

In other aspects of the description provided herein, the HILIC medium need not be in a dry state at the initiation of the loading of the HILIC load to the solid phase extraction column. For example, in some embodiments, the solid phase extraction column is equilibrated, wetted, and/or washed prior to loading a HILIC load to the solid phase extraction column, such as according to manufacturer's instructions. In some embodiments, wherein a HILIC medium is not in a dry state, loading a HILIC load to a solid phase extraction column comprising the HILIC medium is characterized by one or more of: (1) the HILIC load loaded to the solid phase extraction column has an amount of the plurality of proteolytically digested peptides characterized by one or both of: (a) a ratio of the weight of the plurality of proteolytically digested peptides over the weight of the HILIC medium in the dry state of at least about 0.06; and/or (b) a ratio of the weight of the plurality of proteolytically digested peptides relative to the bed volume of the HILIC medium in the dry state of at least about 40 μg/μl; or (2) the HILIC load loaded to the solid phase extraction column has a concentration of an organic solvent of at least about 70% (v/v).

In some embodiments, the HILIC medium comprises a solid phase. In some embodiments, the HILIC medium comprises a solid phase comprising a polar functional moiety. In some embodiments, the solid phase comprises a silica material. In some embodiments, the polar functional moiety comprises one or more of an amino group, a cyano group, a carbamoyl group, an aminoalkyl group, alkylamide group, or a combination thereof. In some embodiments, the HILIC medium comprises an amide resin (e.g., Tosoh TSK-Gel Amide 80 Resin). In some embodiments, the HILIC medium comprises silica particles covalently bonded with carbamoyl groups. In some embodiments, the HILIC medium comprises 2 μm, 3 μm, 5 μm, or 10 μm silica particles of covalently bonded with carbamoyl groups, see, e.g., Y. Kawachi et al., J Chromatography A, 2011, which is incorporated herein by reference in its entirety. In some embodiments, the solid phase extraction column comprises about 0.5 mg to about 5 mg, including about any of 1 mg, 1.5 mg, 2 mg, 2.5 mg, 3 mg, 3.5 mg, 4 mg, or 4.5 mg, of a HILIC medium in a dry state. In some embodiments, the solid phase extraction column comprises a bed volume of a HILIC medium in a dry state of about 2.5 μl to about 7.5 μl, including about 5 μl. In some embodiments, the solid phase extraction column comprises about 2.5 mg to about 3.5 mg, including about 3 mg, of a HILIC medium in a dry state, a bed volume of the HILIC medium in a dry state of about 4 μl to about 6 μl, including about 5 μl, and wherein the HILIC medium comprises silica particles covalently bonded with carbamoyl groups. In some embodiments, the solid phase extraction column is a GlykoPrep Cleanup cartridge (e.g., Product code: GS96-CU; WS0263).

HILIC is used primarily for the separation of polar and hydrophilic compounds such as, for example, glycopeptides. HILIC stationary phases are polar and tend to bind hydrophilic compounds like glycopeptides. Typical mobile phases are aqueous buffers with organic modifiers such as acetonitrile. It is believed that the aqueous content of the liquid passing through the phase creates a water rich layer on the surface of the stationary phase. This allows for partitioning of solutes between the more organic mobile phase and the aqueous layer. Liquid with a higher proportion of organic solvent passing through the HILIC phase tends to cause polar compounds to partition into the aqueous layer on the HILIC medium. In contrast, liquid with a lower proportion of organic solvent passing through the HILIC phase tends to cause polar compounds to partition out of the aqueous layer and into the passing liquid.

### II. HILIC Loads and Methods of Making

Provided herein, in some aspects, is a method for processing a proteolytic digest sample for use in a LC-MS analysis, the method comprising loading a HILIC load to a solid phase extraction column comprising a HILIC medium, wherein the HILIC load comprises: (a) an amount of the plurality of proteolytically digested peptides characterized by one or both of: (i) a ratio of the weight of the plurality of proteolytically digested peptides over the weight of the HILIC medium in the dry state of at least about 0.05, including at least about 0.06; and/or (ii) a ratio of the weight of the plurality of proteolytically digested peptides relative to the bed volume of the HILIC medium in the dry state of at least about 30 μg/μl, including at least about 40 μg/μl; and/or (b) a concentration of an organic solvent of at least about 70% (v/v). Also provided herein are methodologies for making the HILIC load having: (a) an amount of the plurality of proteolytically digested peptides characterized by one or both of: (i) a ratio of the weight of the plurality of proteolytically digested peptides over the weight of the HILIC medium in the dry state of at least about 0.05, including at least about 0.06; and/or (ii) a ratio of the weight of the plurality of proteolytically digested peptides relative to the bed volume of the HILIC medium in the dry state of at least about 30 μg/μl, including at least about 40 μg/μl; and/or (b) a concentration of an organic solvent of at least about 70% (v/v). For example, in some embodiments, the methods provided herein comprise: (a) obtaining a proteolytic digest sample; (b) removing a liquid content of the proteolytic digest sample; and (c) reconstituting the proteolytic digest sample to produce the HILIC load such that the HILIC load comprises: (A) an amount of the plurality of proteolytically digested peptides characterized by one or both of: (i) a ratio of the weight of the plurality of proteolytically digested peptides over the weight of the HILIC medium in the dry state of at least about 0.05, including at least about 0.06; and/or (ii) a ratio of the weight of the plurality of proteolytically digested peptides relative to the bed volume of the HILIC medium in the dry state of at least about 30 μg/μl, including at least about 40 μg/μl; and/or (b) a concentration of an organic solvent of at least about 70% (v/v).

In some embodiments, the HILIC load loaded to a solid phase extraction column has an amount of the plurality of proteolytically digested peptides characterized by a ratio of the weight of the plurality of proteolytically digested peptides over the weight of the HILIC medium in the dry state of about 0.05 to about 0.5, such as any of about 0.05 to about 0.35, about 0.05 to about 0.15, about 0.05 to about 0.25, or about 0.15 to about 0.25. In some embodiments, the HILIC load loaded to a solid phase extraction column has an amount of the plurality of proteolytically digested peptides characterized by a ratio of the weight of the plurality of proteolytically digested peptides over the weight of the HILIC medium in the dry state of at least about 0.05, such as at least about any of 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, or 0.35. In some embodiments, the HILIC load loaded to a solid phase extraction column has an amount of the plurality of proteolytically digested peptides characterized by a ratio of the weight of the plurality of proteolytically digested peptides over the weight of the HILIC medium in the dry state is about any of 0.05, 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, or 0.35. In some embodiments, the HILIC load loaded to a solid phase extraction column has an amount of the plurality of proteolytically digested peptides characterized by a ratio of the weight of the plurality of proteolytically digested peptides over the weight of the HILIC medium in the dry state of at least about 0.1, including about 0.1. In some embodiments, the HILIC load loaded to a solid phase extraction column has an amount of the plurality of proteolytically digested peptides characterized by a ratio of the weight of the plurality of proteolytically digested peptides over the weight of the HILIC medium in the dry state of at least about 0.2, including about 0.2. In any of the embodiments herein, the HILIC medium in the dry state has a weight of about 3 mg. In any of the embodiments herein, the HILIC medium in the dry state has a bed volume of about 5 μL. In some embodiments, the HILIC medium comprises silica particles covalently bonded with carbamoyl groups. In some embodiments, the solid phase extraction column comprises about 2.5 mg to about 3.5 mg, including about 3 mg, of a HILIC medium in a dry state, a bed volume of the HILIC medium in a dry state of about 4 μl to about 6 μl, including about 5 μl, and wherein the HILIC medium comprises silica particles covalently bonded with carbamoyl groups. In some embodiments, the solid phase extraction column is a GlykoPrep Cleanup cartridge (e.g., Product code: GS96-CU; WS0263).

In some embodiments, the HILIC load loaded to the solid phase extraction column has an amount of the plurality of proteolytically digested peptides characterized by a ratio of the weight of the plurality of proteolytically digested peptides relative to the bed volume of the HILIC medium in the dry state of about 30 μg/μl to about 200 μg/μl, such any of about 50 μg/μl to about 130 μg/μl, about 55 μg/μl to about 65 μg/μl, about 110 μg/μl to about 130 μg/μl, or about 115 μg/μl to about 125 μg/μl. In some embodiments, the HILIC load loaded to the solid phase extraction column has an amount of the plurality of proteolytically digested peptides characterized by a ratio of the weight of the plurality of proteolytically digested peptides relative to the bed volume of the HILIC medium in the dry state of at least about 30 μg/μl, such as at least about any of 35 μg/μl, 40 μg/μl, 45 μg/μl, 50 μg/μl, 55 μg/μl, 60 μg/μl, 65 μg/μl, 70 μg/μl, 75 μg/μl, 80 μg/μl, 85 μg/μl, 90 μg/μl, 95 μg/μl, 100 μg/μl, 105 μg/μl, 110 μg/μl, 115 μg/μl, 120 μg/μl, 125 μg/μl, 130 μg/μl, 135 μg/μl, 140 μg/μl, 145 μg/μl, 150 μg/μl, 155 μg/μl, 160 μg/μl, 165 μg/μl, 170 μg/μl, 175 μg/μl, 180 μg/μl, 185 μg/μl, 190 μg/μl, 195 μg/μl, or 200 μg/μl. In some embodiments, the HILIC load loaded to the solid phase extraction column has an amount of the plurality of proteolytically digested peptides characterized by a ratio of the weight of the plurality of proteolytically digested peptides relative to the bed volume of the HILIC medium in the dry state of about any of 30 μg/μl, 35 μg/μl, 40 μg/μl, 45 μg/μl, 50 μg/μl, 55 μg/μl, 60 μg/μl, 65 μg/μl, 70 μg/μl, 75 μg/μl, 80 μg/μl, 85 μg/μl, 90 μg/μl, 95 μg/μl, 100 μg/μl, 105 μg/μl, 110 μg/μl, 115 μg/μl, 120 μg/μl, 125 μg/μl, 130 μg/μl, 135 μg/μl, 140 μg/μl, 145 μg/μl, 150 μg/μl, 155 μg/μl, 160 μg/μl, 165 μg/μl, 170 μg/μl, 175 μg/μl, 180 μg/μl, 185 μg/μl, 190 μg/μl, 195 μg/μl, or 200 μg/μl. In some embodiments, the HILIC load loaded to the solid phase extraction column has an amount of the plurality of proteolytically digested peptides characterized by a ratio of the weight of the plurality of proteolytically digested peptides relative to the bed volume of the HILIC medium in the dry state of at least about 60 μg/μl, including about 60 μg/μl. In some embodiments, the HILIC load loaded to the solid phase extraction column has an amount of the plurality of proteolytically digested peptides characterized by a ratio of the weight of the plurality of proteolytically digested peptides relative to the bed volume of the HILIC medium in the dry state of at least about 120 μg/μl, including about 120 μg/μl. In any of the embodiments herein, the HILIC medium in the dry state has a weight of about 3 mg. In any of the embodiments herein, the HILIC medium in the dry state has a bed volume of about 5 μL. In some embodiments, the HILIC medium comprises silica particles covalently bonded with carbamoyl groups. In some embodiments, the solid phase extraction column comprises about 2.5 mg to about 3.5 mg, including about 3 mg, of a HILIC medium in a dry state, a bed volume of the HILIC medium in a dry state of about 4 μl to about 6 μl, including about 5 μl, and wherein the HILIC medium comprises silica particles covalently bonded with carbamoyl groups. In some embodiments, the solid phase extraction column is a GlykoPrep Cleanup cartridge (e.g., Product code: GS96-CU; WS0263).

In some embodiments, the HILIC load loaded to a solid phase extraction column has an amount of the plurality of proteolytically digested peptides characterized by a ratio of the weight of the plurality of proteolytically digested peptides over the weight of the HILIC medium in the dry state of about 0.05 to about 0.5, such as any of about 0.05 to about 0.35, about 0.05 to about 0.15, about 0.05 to about 0.25, or about 0.15 to about 0.25, and characterized by a ratio of the weight of the plurality of proteolytically digested peptides relative to the bed volume of the HILIC medium in the dry state of about 30 μg/μl to about 200 μg/μl, such any of about 50 μg/μl to about 130 μg/μl, about 55 μg/μl to about 65 μg/μl, about 110 μg/μl to about 130 μg/μl, or about 115 μg/μl to about 125 μg/μl. In some embodiments, the HILIC load loaded to a solid phase extraction column has an amount of the plurality of proteolytically digested peptides characterized by a ratio of the weight of the plurality of proteolytically digested peptides over the weight of the HILIC medium in the dry state of at least about 0.05, such as at least about any of 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, or 0.35, and characterized by a ratio of the weight of the plurality of proteolytically digested peptides relative to the bed volume of the HILIC medium in the dry state of at least about 30 μg/μl, such as at least about any of 35 μg/μl, 40 μg/μl, 45 μg/μl, 50 μg/μl, 55 μg/μl, 60 μg/μl, 65 μg/μl, 70 μg/μl, 75 μg/μl, 80 μg/μl, 85 μg/μl, 90 μg/μl, 95 μg/μl, 100 μg/μl, 105 μg/μl, 110 μg/μl, 115 μg/μl, 120 μg/μl, 125 μg/μl, 130 μg/μl, 135 μg/μl, 140 μg/μl, 145 μg/μl, 150 μg/μl, 155 μg/μl, 160 μg/μl, 165 μg/μl, 170 μg/μl, 175 μg/μl, 180 μg/μl, 185 μg/μl, 190 μg/μl, 195 μg/μl, or 200 μg/μl. In some embodiments, the HILIC load loaded to a solid phase extraction column has an amount of the plurality of proteolytically digested peptides characterized by a ratio of the weight of the plurality of proteolytically digested peptides over the weight of the HILIC medium in the dry state is about any of 0.05, 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, or 0.35, and characterized by a ratio of the weight of the plurality of proteolytically digested peptides relative to the bed volume of the HILIC medium in the dry state of about any of 30 μg/μl, 35 μg/μl, 40 μg/μl, 45 μg/μl, 50 μg/μl, 55 μg/μl, 60 μg/μl, 65 μg/μl, 70 μg/μl, 75 μg/μl, 80 μg/μl, 85 μg/μl, 90 μg/μl, 95 μg/μl, 100 μg/μl, 105 μg/μl, 110 μg/μl, 115 μg/μl, 120 μg/μl, 125 μg/μl, 130 μg/μl, 135 μg/μl, 140 μg/μl, 145 μg/μl, 150 μg/μl, 155 μg/μl, 160 μg/μl, 165 μg/μl, 170 μg/μl, 175 μg/μl, 180 μg/μl, 185 μg/μl, 190 μg/μl, 195 μg/μl, or 200 μg/μl. In some embodiments, the HILIC load loaded to a solid phase extraction column has an amount of the plurality of proteolytically digested peptides characterized by a ratio of the weight of the plurality of proteolytically digested peptides over the weight of the HILIC medium in the dry state of at least about 0.1, including about 0.1, and characterized by a ratio of the weight of the plurality of proteolytically digested peptides relative to the bed volume of the HILIC medium in the dry state of at least about 60 μg/μl, including about 60 μg/μl. In some embodiments, the HILIC load loaded to a solid phase extraction column has an amount of the plurality of proteolytically digested peptides characterized by a ratio of the weight of the plurality of proteolytically digested peptides over the weight of the HILIC medium in the dry state of at least about 0.2, including about 0.2, and characterized by a ratio of the weight of the plurality of proteolytically digested peptides relative to the bed volume of the HILIC medium in the dry state of at least about 120 μg/μl, including about 120 μg/μl. In any of the embodiments herein, the HILIC medium in the dry state has a weight of about 3 mg. In any of the embodiments herein, the HILIC medium in the dry state has a bed volume of about 5 μL. In some embodiments, the HILIC medium comprises silica particles covalently bonded with carbamoyl groups. In some embodiments, the solid phase extraction column comprises about 2.5 mg to about 3.5 mg, including about 3 mg, of a HILIC medium in a dry state, a bed volume of the HILIC medium in a dry state of about 4 μl to about 6 μl, including about 5 μl, and wherein the HILIC medium comprises silica particles covalently bonded with carbamoyl groups. In some embodiments, the solid phase extraction column is a GlykoPrep Cleanup cartridge (e.g., Product code: GS96-CU; WS0263).

In some embodiments, the HILIC load has an amount of a plurality of proteolytically digested peptides of about 150 μg to about 1,000 μg, such as any of about 200 μg to about 800 μg, about 200 μg to about 600 μg, about 250 μg to about 650 μg, or about 300 μg to about 600 μg. In some embodiments, the HILIC load has an amount of a plurality of proteolytically digested peptides of at least about 150 μg, such as at least about any of 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, or 1,000 μg. In some embodiments, the HILIC load has an amount of a plurality of proteolytically digested peptides of about 200 μg or more, such as about any of 250 μg or more, 300 μg or more, 350 μg or more, 400 μg or more, 450 μg or more, 500 μg or more, 550 μg or more, 600 μg or more, 650 μg or more, 700 μg or more, 750 μg or more, 800 μg or more, 850 μg or more, 900 μg or more, 950 μg or more, or 1,000 μg or more.

In some embodiments, the HILIC load has an amount of a plurality of proteolytically digested peptides of about 150 μg to about 1,000 μg, such as any of about 200 μg to about 800 μg, about 200 μg to about 600 μg, about 250 μg to about 650 μg, or about 300 μg to about 600 μg, wherein the solid phase extraction column comprises about 2.5 mg to about 3.5 mg, including about 3 mg, of a HILIC medium in a dry state, a bed volume of the HILIC medium in a dry state of about 4 μl to about 6 μl, including about 5 μl, and wherein the HILIC medium comprises silica particles covalently bonded with carbamoyl groups. In some embodiments, the HILIC load has an amount of a plurality of proteolytically digested peptides of at least about 150 μg, such as at least about any of 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, or 1,000 μg, wherein the solid phase extraction column comprises about 2.5 mg to about 3.5 mg, including about 3 mg, of a HILIC medium in a dry state, a bed volume of the HILIC medium in a dry state of about 4 μl to about 6 μl, including about 5 μl, and wherein the HILIC medium comprises silica particles covalently bonded with carbamoyl groups. In some embodiments, the HILIC load has an amount of a plurality of proteolytically digested peptides of about 200 μg or more, such as about any of 250 μg or more, 300 μg or more, 350 μg or more, 400 μg or more, 450 μg or more, 500 μg or more, 550 μg or more, 600 μg or more, 650 μg or more, 700 μg or more, 750 μg or more, 800 μg or more, 850 μg or more, 900 μg or more, 950 μg or more, or 1,000 μg or more, wherein the solid phase extraction column comprises about 2.5 mg to about 3.5 mg, including about 3 mg, of a HILIC medium in a dry state, a bed volume of the HILIC medium in a dry state of about 4 μl to about 6 μl, including about 5 μl, and wherein the HILIC medium comprises silica particles covalently bonded with carbamoyl groups. In some embodiments, the solid phase extraction column is a GlykoPrep Cleanup cartridge (e.g., Product code: GS96-CU; WS0263).

Polypeptide amounts described herein may be absolute or estimated amounts. In some embodiments, the amount of polypeptide content in any sample, or a derivative thereof, is a measured directly from said sample, or the derivative thereof, e.g., using a BCA quantification assay or a UV-VIS measurement at 280 nm. In some embodiments, the amount of polypeptide content in a sample, or a derivative thereof, is estimated based on a known, including reference standard, value for polypeptide content in the sample based on the origin of the sample, e.g., such as based on a known standard polypeptide concentration in human plasma or serum.

In some embodiments, provided is a method for processing a proteolytic digest sample for use in a LC-MS analysis, the method comprising loading a HILIC load to a solid phase extraction column comprising a HILIC medium, wherein the HILIC load comprises a concentration of an organic solvent of at least about 70% (v/v), such as at least about any of 71% (v/v), 72% (v/v), 73% (v/v), 74% (v/v), 75% (v/v), 76% (v/v), 77% (v/v), 78% (v/v), 79% (v/v), 80% (v/v), 81% (v/v), 82% (v/v), 83% (v/v), 84% (v/v), 85% (v/v), 86% (v/v), 87% (v/v), 88% (v/v), 89% (v/v), 90% (v/v), 91% (v/v), 92% (v/v), 93% (v/v), 94% (v/v), 95% (v/v), 96% (v/v), 97% (v/v), 98% (v/v), or 99% (v/v). In some embodiments, the HILIC load comprises a concentration of an organic solvent of about any of 70% (v/v), 71% (v/v), 72% (v/v), 73% (v/v), 74% (v/v), 75% (v/v), 76% (v/v), 77% (v/v), 78% (v/v), 79% (v/v), 80% (v/v), 81% (v/v), 82% (v/v), 83% (v/v), 84% (v/v), 85% (v/v), 86% (v/v), 87% (v/v), 88% (v/v), 89% (v/v), 90% (v/v), 91% (v/v), 92% (v/v), 93% (v/v), 94% (v/v), 95% (v/v), 96% (v/v), 97% (v/v), 98% (v/v), 99% (v/v), or 100% (v/v).

In some embodiments, the organic solvent comprises an aprotic solvent miscible in water. In some embodiments, the organic solvent is selected from the group consisting of acetonitrile, ethanol, methanol, tetrahydrofuran, and dioxane, or a combination thereof. In some embodiments, the organic solvent comprises acetonitrile (ACN). In some embodiments, the organic solvent is ACN.

In some embodiments, provided is a method for processing a proteolytic digest sample for use in a LC-MS analysis, the method comprising loading a HILIC load to a solid phase extraction column comprising a HILIC medium, wherein the HILIC load comprises a concentration of ACN of at least about 70% (v/v), such as at least about any of 71% (v/v), 72% (v/v), 73% (v/v), 74% (v/v), 75% (v/v), 76% (v/v), 77% (v/v), 78% (v/v), 79% (v/v), 80% (v/v), 81% (v/v), 82% (v/v), 83% (v/v), 84% (v/v), 85% (v/v), 86% (v/v), 87% (v/v), 88% (v/v), 89% (v/v), 90% (v/v), 91% (v/v), 92% (v/v), 93% (v/v), 94% (v/v), 95% (v/v), 96% (v/v), 97% (v/v), 98% (v/v), or 99% (v/v). In some embodiments, the HILIC load comprises a concentration of ACN of about any of 70% (v/v), 71% (v/v), 72% (v/v), 73% (v/v), 74% (v/v), 75% (v/v), 76% (v/v), 77% (v/v), 78% (v/v), 79% (v/v), 80% (v/v), 81% (v/v), 82% (v/v), 83% (v/v), 84% (v/v), 85% (v/v), 86% (v/v), 87% (v/v), 88% (v/v), 89% (v/v), 90% (v/v), 91% (v/v), 92% (v/v), 93% (v/v), 94% (v/v), 95% (v/v), 96% (v/v), 97% (v/v), 98% (v/v), 99% (v/v), or 100% (v/v). In some embodiments, the HILIC load comprises a concentration of ACN of about 80% (v/v).

In some aspects of the methods provided herein, the method comprises one or more steps for obtaining, such as making, a HILIC load. In some embodiments, the HILIC load comprises: (a) an amount of the plurality of proteolytically digested peptides characterized by one or both of: (i) a ratio of the weight of the plurality of proteolytically digested peptides over the weight of the HILIC medium in the dry state of at least about 0.05; and/or (ii) a ratio of the weight of the plurality of proteolytically digested peptides relative to the bed volume of the HILIC medium in the dry state of at least about 30 μg/μl, including such HILIC load in a set volume, e.g., about 220 μl or less; and/or (b) a concentration of an organic solvent of at least about 70% (v/v). In some embodiments, the proteolytic digest sample, such as resulting from a tryptic digestion, is of a different constitution than a HILIC load used in the methods described herein, such as HILIC load comprising (a) an amount of the plurality of proteolytically digested peptides characterized by one or both of: (i) a ratio of the weight of the plurality of proteolytically digested peptides over the weight of the HILIC medium in the dry state of at least about 0.05; and/or (ii) a ratio of the weight of the plurality of proteolytically digested peptides relative to the bed volume of the HILIC medium in the dry state of at least about 30 μg/μl; and/or (b) a concentration of an organic solvent of at least about 70% (v/v). In such embodiments, encompassed herein are method steps for processing a proteolytic digest sample to form a HILIC load. In some embodiments, the methods provided herein comprise: (a) obtaining a proteolytic digest sample; (b) removing a liquid content of the proteolytic digest sample; and (c) reconstituting the proteolytic digest sample to produce the HILIC load such that the HILIC load comprises: (A) an amount of the plurality of proteolytically digested peptides characterized by one or both of: (i) a ratio of the weight of the plurality of proteolytically digested peptides over the weight of the HILIC medium in the dry state of at least about 0.05; and/or (ii) a ratio of the weight of the plurality of proteolytically digested peptides relative to the bed volume of the HILIC medium in the dry state of at least about 30 μg/μl, including such HILIC load in a set volume, e.g., about 220 μl or less; and/or (B) a concentration of an organic solvent of at least about 70% (v/v).

In some embodiments, obtaining the HILIC load comprises reducing (such as removing) a liquid content from a proteolytic digest sample, such as resulting from a tryptic digestion, without substantial loss of a plurality of proteolytically digested peptides in the proteolytic digest sample. In some embodiments, the HILIC load comprises at least about 70%, 80%, or 90%, such as at least about any of 91%, 92%, 93%, 94%, 95%, 96%, 97%, 99%, or 100%, of a plurality of proteolytically digested peptides as compared to a proteolytic digest sample from which the HILIC load is obtained. In some embodiments, the reducing (such as removing) the liquid content from the proteolytic digested sample comprises performing a peptide concentrating technique with the proteolytically digested sample to obtain a precursor of the HILIC load such that (a) the precursor can be reconstituted with a reconstitution liquid comprising the organic solvent to obtain the HILIC load having a volume of 220 μL or less and a concentration of the organic solvent of at least about 70% (v/v); and (b) the resulting HILIC load comprises an amount of the plurality of proteolytically digested peptides of at least about 200 g.

In some embodiments, the method comprises reducing a liquid content from the proteolytic digest sample to form a dried proteolytic digest sample; and reconstituting the dried proteolytic digest sample with a reconstitution liquid comprising the organic solvent to produce the HILIC load such that (a) the HILIC load has a volume of about 220 μL or less and a concentration of the organic solvent of at least about 70% (v/v); and (b) the HILIC load has an amount of the plurality of proteolytic peptides of at least about 200 μg.

In some embodiments, the reconstituting the dried proteolytic digest sample comprises: mixing the dried proteolytic digest sample with an amount of water to form a water mixture: sonicating the water mixture with a sonicator; mixing the water mixture with an amount of trifluoracetic acid (TFA) and acetonitrile (ACN), wherein the amount of TFA and ACN are such that the final concentration of TFA is about 1% (v/v) and the final concentration of ACN is about 80% (v/v); and sonicating the water mixture having the amount of TFA and ACN with a sonicator to produce the HILIC load. In some embodiments, the sonicating the water mixture with the sonicator comprises a water-based dissolution cycle, wherein the water-based dissolution cycle is repeated about 2 times to about 5 times, and wherein for each of the water-based dissolution cycles, the sonicating the water mixture is performed for about 5 minutes and a water reservoir of the sonicator is configured with ice to cool the water reservoir. In some embodiments, the sonicating the water mixture having the amount of TFA and ACN with the sonicator comprises an organic-based dissolution cycle, wherein the organic-based dissolution cycle is repeated about 2 times to about 3 times, and wherein for each of the organic-based dissolution cycles, the sonicating is performed for about 4 minutes and a water reservoir of the sonicator is configured with ice to cool the water reservoir.

In some embodiments, wherein the reducing (such as removing) the liquid content from the proteolytic digest sample comprises removing all or substantially all of the liquid content therefrom. In some embodiments, the peptide concentrating technique comprises a vacuum evaporation technique or a lyophilization technique. In some embodiments, the peptide concentrating technique comprises a SpeedVac.

In some embodiments, the proteolytic digest sample having reduced liquid content, such as a dried proteolytic digest sample, is reconstituted with one or more liquids. In some embodiments, the one or more liquids comprise one or more of water, TFA, ACN, or any combination thereof. In some embodiments, the dried proteolytic digest sample is reconstituted in stages, such as a first stage with water and a second stage with TFA and ACN, such that a HILIC load is obtained as described herein. In some embodiments, the volume of the HILIC load is about 10 μL to about 1 mL, such as about 100 to about 500 μL, about 50 μL to about 220 μL, or about 100 to about 220 μL. In some embodiments, the volume of the HILIC load is about 220 μL or less, such as about 210 μL or less, 200 μL or less, 190 μL or less, 180 μL or less, 170 μL or less, 160 μL or less, 150 μL or less, 140 μL or less, 130 μL or less, 120 μL or less, 110 μL or less, 100 μL or less, 90 μL or less, 80 μL or less, 70 μL or less, 60 μL or less, or 50 μL or less.

### III. Additional Aspects of the Methods for Processing a Proteolytic Digest Sample

In certain aspects, the method provided herein comprises one or more additional steps involved with processing a proteolytic digest sample taught herein.

In some embodiments, the method comprises performing a washing step after loading a HILIC load to a solid phase extraction column comprising a HILIC medium and prior to subjecting the HILIC medium to n elution liquid, wherein the washing step comprises subjecting the HILIC medium to a wash liquid. In some embodiments, the wash liquid comprises an organic solvent at a concentration of at least about 90% (v/v), such as at least about 91% (v/v), 92% (v/v), 93% (v/v), 94% (v/v), 95% (v/v), 96% (v/v), 97% (v/v), 98% (v/v), or 99% (v/v). In some embodiments, the organic solvent is ACN. In some embodiments, the wash liquid further comprises TFA at a concentration of about 0.5% (v/v) to about 1% (v/v), including 1% (v/v).

In some embodiments, the elution liquid comprises a high percentage of an aqueous liquid, such as at least about 90% (v/v), such as at least about 91% (v/v), 92% (v/v), 93% (v/v), 94% (v/v), 95% (v/v), 96% (v/v), 97% (v/v), 98% (v/v), or 99% (v/v), of water. In some embodiments, the elution liquid further comprises about 0.05% (v/v) to about 0.5% (v/v) of TFA, such as about 0.1% TFA.

In some embodiments, the method comprises collecting the HILIC eluate, or a fraction thereof, from the solid phase extraction column, wherein the HILIC eluate comprises the at least one proteolytically digested glycopeptide. In some embodiments, after the collecting the HILIC eluate from the solid phase extraction column, the method further comprises reducing a liquid content of the collected HILIC eluate. In some embodiments, the method comprises subjecting the HILIC eluate to a peptide concentrating technique to produce a dried HILIC eluate, such as a peptide concentrating technique (e.g., SpeedVac).

In some embodiments, the method comprises reconstituting the dried HILIC eluate to form a sample suitable for introduction to the LC-MS system. In some embodiments, the dried HILIC eluate is reconstituted using 0.1% formic acid (FA) or TFA in water (e.g., in 30 μL of 0.1% FA in water).

As described in more detail herein, in some embodiments, the method comprises injecting the sample suitable for introduction to the LC-MS system into the LC-MS system. In some embodiments, the method comprises performing a mass spectrometry technique to obtain mass spectrometry data. In some embodiments, the method comprises identifying a peptide sequence of a glycopeptide from the mass spectrometry data. In some embodiments, the method comprises identifying a glycan attachment site of the glycopeptide from the mass spectrometry data. In some embodiments, the method comprises identifying a glycan structure of the glycopeptide from the mass spectrometry data. In some embodiments, the at least one glycopeptide comprises a glycan structure comprising one or more sialic acid moieties. In some embodiments, the proteolytic digest sample is obtained from a method for proteolytically digesting a biological sample comprising a glycoprotein.

In certain aspects, the method provided herein enable the enrichment of glycopeptide species derived from a biological sample, such as via a proteolytic digest sample. In some embodiments, a glycopeptide concentration for a glycopeptide derived from the proteolytic digest sample is enriched by a factor of 30 or greater, such as any of 40 or greater, 50 or greater, 60 or greater, 70 or greater, 80 or greater, 90 or greater, 100 or greater, 110 or greater, 120 or greater, 130 or greater, 140 or greater, 150 or greater, 160 or greater, 170 or greater, 180 or greater, 190 or greater, or 200 or greater, with respect to a peptide concentration, wherein the peptide concentration represents an amount of a peptide that is associated with the same protein as the glycopeptide. In some embodiments, the method comprises: measuring a first plurality of peak area values for a first panel of glycopeptides; measuring a second plurality of peak area values for a second panel of unglycosylated peptides wherein each of the unglycosylated peptides of the second panel corresponds to each of the glycopeptides of the first panel by being attached to a same protein molecule before a proteolytic digestion; calculating a plurality of ratios by dividing each of the first plurality of peak area values with each of the second plurality of peak area values, respectively; and determining a median ratio from the plurality of ratios, wherein the median ratio is greater than 30.

In some embodiments, wherein the method for processing a proteolytic digest sample is performed in replicate (e.g., two or more aliquots of a proteolytic digest sample are processed using the same method for processing), the resulting coefficient of variation (CV) of a peak feature, such as peak area of the measured polypeptide, e.g., glycopolypeptide, is about 15% or less, such as about any of 14% or less, 13% or less, 12% or less, 11% or less, 10% or less, 9% or less, 8% or less, 7% or less, 6% or less, 5% or less, 4% or less, 3% or less, 2% or less, or 1% or less.

### D5. Methods for Proteolytically Digesting a Biological Sample Comprising a Glycoprotein

In certain aspects, provided herein are methods of proteolytically digesting a biological sample comprising a glycoprotein, the methods comprising subjecting the biological sample to a thermal denaturation technique. Proteases are enzymes that cleave polypeptides at, generally, specific cleavage motifs. For example, trypsin is a serine protease that generally cleaves polypeptides at the carboxyl side (C-terminal side) of lysine and arginine residues. A glycan of a glycopeptide may present a steric hindrance to a protease, thereby inhibiting complete protease digestion of a biological sample comprising a glycoprotein. Without being bound to this theory, it is believed that the methods taught herein improve polypeptide unfolding, such as linearization, and provide protease access to cleavage sites thereby providing methods for more complete proteolytic digestion of glycoproteins.

In some aspects, provided is a method comprising subjecting a biological sample to a thermal denaturation technique to produce a denatured sample.

In other aspects, provided is a method comprising subjecting a biological sample to a thermal denaturation technique to produce a denatured sample followed by a proteolytic digestion technique to produce a proteolytic digest sample comprising a proteolytic glycopeptide. In some embodiments, the method comprises quenching one or more proteases used in a proteolytic digestion technique prior to a downstream technique, such as LC-MS.

In other aspects, provided herein is a method comprising: subjecting a biological sample to a thermal denaturation technique to produce a denatured sample; subjecting the denatured sample to a reduction technique to produce a reduced sample; subjecting the reduced sample to an alkylation technique to produce an alkylated sample; and subjecting the alkylated sample to a proteolytic digestion technique to produce a proteolytic digest sample comprising the proteolytic glycopeptide. In some embodiments, the method comprises quenching an alkylating agent used in the alkylation technique prior to subjecting an alkylated sample to a proteolytic digestion technique. In some embodiments, the method comprises quenching one or more proteases used in a proteolytic digestion technique prior to a downstream technique, such as LC-MS.

In some embodiments, the proteolytic digestion methods described herein produce a proteolytic digestion sample, including one comprising a proteolytic glycopeptide, having a digestion completion rate of at least about 70%, such as at least about any of 75%, 80%, 85%, 90%, 91%, 92%, 93%, 94%, 95%, 96%, 97%, 98%, or 99%. In some embodiments, the proteolytic digestion methods described herein produce a proteolytic digestion sample, including one comprising a proteolytic glycopeptide, wherein the sample volume loss is 10% or less, such as 9% or less, 8% or less, 7% or less, 6% or less, 5% or less, 4% or less, 3% or less, 2% or less, or 1% or less, based on all volumes added in producing the proteolytic digestion sample.

In the following sections, additional description of the various aspects of the proteolytic digestion techniques is provided. Such description in a modular fashion is not intended to limit the scope of the disclosure, and based on the teachings provided herein one of ordinary skill in the art will readily appreciate that certain modules can be integrated, at least in part. The section heading used herein are for organizational purposes only and are not to be construed as limiting the subject matter described.

### I. Thermal Denaturation Techniques

In certain aspects, the methods provided herein comprise performing a thermal denaturation technique. Thermal denaturation techniques, generally speaking, change certain polypeptides conformational structures, such as by unfolding and/or linearizing a polypeptide, to enable protease access to cleavage sites. Thermal denaturation techniques described herein comprise subjecting a sample, or a derivative thereof (e.g., a sample diluted with a buffer), to a thermal treatment of about 60° C. to about 100° C. for thermal denaturation incubation time of at least about 1 minute. In some embodiments, the thermal denaturation technique is not performed concurrently with a chemical denaturation technique, such as using high concentrations of denaturing agent, e.g., 6M urea. In some embodiments, the method does not include use of a chemical denaturation technique.

In some embodiments, the thermal denaturation incubation time is performed at a temperature of about 60° C. to about 100° C., such as any of about 70° C. to about 100° C., about 80° C. to about 100° C., about 90° C. to about 100° C., about 95° C. to about 100° C., or about 85° C. to about 95° C. In some embodiments, the thermal denaturation incubation time is performed at a temperature of at least about 60° C., such as at least about any of 65° C., 70° C., 75° C., 80° C., 85° C., 90° C., 95° C., or 100° C. In some embodiments, the thermal denaturation incubation time is performed at a temperature of about 100° C. or less, such as about any of 95° C. or less, 90° C. or less, 85° C. or less, 80° C. or less, 75° C. or less, 70° C. or less, 65° C. or less, 60° C. or less. In some embodiments, the thermal denaturation incubation time is performed at a temperature of about any of 60° C., 65° C., 70° C., 75° C., 80° C., 85° C., 90° C., 91° C., 92° C., 93° C., 94° C., 95° C., 96° C., 97° C., 98° C., 99° C., or 100° C.

In some embodiments, the thermal denaturation incubation time is about 1 minute to about 15 minutes, such as any of about 1 minute to about 5 minutes, about 1 minute to about 10 minutes, about 2.5 minutes to about 7.5 minutes, or about 5 minutes to about 15 minutes. In some embodiments, the thermal denaturation incubation time is at least about 1 minute, such as at least about any of 1.5 minutes, 2 minutes, 2.5 minutes, 3 minutes, 3.5 minutes, 4 minutes, 4.5 minutes, 5 minutes, 5.5 minutes, 6 minutes, 6.5 minutes, 7 minutes, 7.5 minutes, 8 minutes, 8.5 minutes, 9 minutes, 9.5 minutes, 10 minutes, 11 minutes, 12 minutes, 13 minutes, 14 minutes, or 15 minutes. In some embodiments, the thermal denaturation incubation time is about 15 minutes or less, such as about any of 14 minutes or less, 13 minutes or less, 12 minutes or less, 11 minutes or less, 10 minutes or less, 9.5 minutes or less, 9 minutes or less, 8.5 minutes or less, 8 minutes or less, 7.5 minutes or less, 7 minutes or less, 6.5 minutes or less, 6 minutes or less, 5.5 minutes or less, 5 minutes or less, 4.5 minutes or less, 4 minutes or less, 3.5 minutes or less, 3 minutes or less, 2.5 minutes or less, 2 minutes or less, 1.5 minutes or less, or 1 minute or less. In some embodiments, the thermal denaturation incubation time is about any of 1 minute, 1.5 minutes, 2 minutes, 2.5 minutes, 3 minutes, 3.5 minutes, 4 minutes, 4.5 minutes, 5 minutes, 5.5 minutes, 6 minutes, 6.5 minutes, 7 minutes, 7.5 minutes, 8 minutes, 8.5 minutes, 9 minutes, 9.5 minutes, 10 minutes, 11 minutes, 12 minutes, 13 minutes, 14 minutes, or 15 minutes.

In some embodiments, the thermal denaturation technique comprises a thermal denaturation incubation time of about 1 minute to about 15 minutes, such as about any of 1 minute, 2 minutes, 3 minutes, 4 minutes, 5 minutes, 6 minutes, 7 minutes, 8 minutes, 9 minutes 10 minutes, 11 minutes, 12 minutes, 13 minutes, 14 minutes, or 15 minutes, wherein the thermal denaturation incubation is performed at a temperature of about 60° C. to about 100° C., such about any of 60° C., 65° C., 70° C., 75° C., 80° C., 85° C., 90° C., 91° C., 92° C., 93° C., 94° C., 95° C., 96° C., 97° C., 98° C., 99° C., or 100° C.

The thermal denaturation incubation temperature can be controlled by numerous techniques and combinations thereof. In some embodiments, the thermal denaturation incubation temperature is controlled by a water bath. In some embodiments, the thermal denaturation incubation temperature is controlled by a heat block. In some embodiments, the thermal denaturation incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the thermal denaturation incubation time temperature are controlled by a technique described herein, such as cooling at room temperature or via the thermocycler.

In some embodiments, the thermal denaturation technique comprises subjecting a sample, or a derivative thereof, e.g., a sample diluted in a buffer, to a thermal cycle. In some embodiments, the thermal cycle comprises subjecting the sample, or a derivative thereof, to one or more of: (a) a block starting temperature (b) block set temperature (the temperature for the thermal denaturation incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 60° C. to about 100° C., including about any of 60° C., 65° C., 70° C., 75° C., 80° C., 85° C., 90° C., 91° C., 92° C., 93° C., 94° C., 95° C., 96° C., 97° C., 98° C., 99° C., or 100° C. In some embodiments, the thermal cycle comprises: (a) a set block temperature of about 60° C. to about 100° C., including about any of 60° C., 65° C., 70° C., 75° C., 80° C., 85° C., 90° C., 91° C., 92° C., 93° C., 94° C., 95° C., 96° C., 97° C., 98° C., 99° C., or 100° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 50° C., such as any of 15° C. to about 25° C., about 20° C. to about 30° C., or about 20° C. to about 25° C., including about any of 20° C., 21° C., 22° C., 23° C., 24° C., 25° C., (b) a set block temperature of about 60° C. to about 100° C., such about any of 60° C., 65° C., 70° C., 75° C., 80° C., 85° C., 90° C., 91° C., 92° C., 93° C., 94° C., 95° C., 96° C., 97° C., 98° C., 99° C., or 100° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C. In some embodiments, the lid temperature during the thermal cycle may be the same respective temperature of the block during the thermal cycle or a temperature greater than the temperature of the block during the thermal cycle. In some embodiments, the lid temperature during the thermal cycle may be the same respective temperature of the block during the thermal cycle or a temperature greater than the temperature of the block during the thermal cycle.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

In some embodiments, the method further comprises admixing an amount of a biological sample a buffer prior to the thermal denaturation technique (e.g., the buffered sample is subjected to a thermal denaturation technique described herein). In some embodiment, the amount (as assessed based on the final concentration in the sample containing solution containing solution) of the buffer is about 1 mM to about 100 mM, such as any of about 20 mM to about 80 mM, about 30 mM to about 70 mM, or about 40 mM to about 60 mM. In some embodiment, the amount of the buffer is about any of 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some embodiments, the buffer is selected from the group consisting of ammonium bicarbonate, ammonium acetate, ammonium formate, triethylammonium bicarbonate, and Tris-HCl, or any combination thereof.

In some embodiments, the method further comprises determining the protein concentration in a biological sample or a derivative thereof.

### II. Reduction Techniques

In certain aspects, the methods provided herein comprise performing a reduction technique. In some embodiments, the reduction technique is performed on a sample, or a derivative thereof, following thermal denaturation. Reduction techniques, generally speaking, reduce (e.g., cleave) disulfide linkages between cysteine residues of one or more polypeptides to reduce the presence of polypeptide conformations that inhibit or prevent protease cleavage of the one or more polypeptides. Reduction techniques described herein comprise subjecting a sample, or a derivative thereof (e.g., a denatured sample), to an amount of a reducing agent and incubating for a reducing incubation time performed at a temperature or range thereof.

In some embodiments, the reducing agent is dithiothreitol (DTT), tris(2-carboxyethyl) phosphine (TCEP), beta-mercaptoethanol (BME), or a cysteine, or any mixture thereof.

In some embodiments, the amount (as assessed based on the final concentration in the sample containing solution containing solution) of a reducing agent, e.g., DTT, used in a reduction technique is about 1 mM to about 100 mM, such as any of about 1 mM to about 40 mM, about 1 mM to about 30 mM, about 5 mM to about 25 mM, about 10 mM, to about 20 mM, 20 mM to about 80 mM, about 30 mM to about 70 mM, or about 40 mM to about 60 mM. In some embodiments, the amount of reducing agent used in a reduction technique is at least about 1 mM, such as at least about any of 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some embodiments, the amount of reducing agent used in a reduction technique is about 100 mM or less, such as about any of 95 mM or less, 90 mM or less, 85 mM or less, 80 mM or less, 75 mM or less, 70 mM or less, 65 mM or less, 60 mM or less, 55 mM or less, 50 mM or less, 45 mM or less, 40 mM or less, 35 mM or less, 30 mM or less, 25 mM or less, 20 mM or less, 19 or less, 18 or less, 17 or less, 16 or less, 15 or less, 14 or less, 13 or less, 12 or less, 11 or less, 10 or less, 9 or less, 8 or less, 7 or less, 6 or less, 5 or less, 4 or less, 3 or less, 2 or less, or 1 or less. In some embodiments, the amount of reducing agent used in a reduction technique is about any of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In some embodiments, the reduction incubation time is about 10 minutes to about 120 minutes, such as any of about 30 minutes to about 60 minutes, about 40 minutes to about 60 minutes, about 45 minutes to about 55 minutes. In some embodiments, the reduction incubation time is at least about 20 minutes, such as at least about any of 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes. In some embodiments, the reduction incubation time is about 120 minutes or less, such as about any of 115 minutes or less, 110 minutes or less, 105 minutes or less, 100 minutes or less, 95 minutes or less, 90 minutes or less, 85 minutes or less, 80 minutes or less, 75 minutes or less, 70 minutes or less, 65 minutes or less, 60 minutes or less, 55 minutes or less, 50 minutes or less, 45 minutes or less, 40 minutes or less, 35 minutes or less, 30 minutes or less, or 25 minutes or less. In some embodiments, the reduction incubation time is about any of 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes.

In some embodiments, the reduction incubation time is performed at a temperature of about 20° C. to about 100° C., such as any of about 40° C. to about 80° C., about 50° C. to about 70° C., about 50° C. to about 60° C., about 55° C. to about 65° C., or about 60° C. to about 70° C. In some embodiments, the reduction incubation time is performed at a temperature of at least about 20° C., such as at least about any of 25° C., 30° C., 35° C., 40° C., 45° C., 50° C., 55° C., 60° C., 65° C., 70° C., 75° C., 80° C., 85° C., 90° C., or 95° C. In some embodiments, the reduction incubation time is performed at a temperature of about 95° C. or less, such as about any of 90° C. or less, 85° C. or less, 80° C. or less, 75° C. or less, 70° C. or less, 65° C. or less, 60° C. or less, 55° C. or less, 50° C. or less, 45° C. or less, 40° C. or less, 35° C. or less, 30° C. or less, or 25° C. or less. In some embodiments, the reduction incubation time is performed at a temperature of about any of 20° C., 25° C., 30° C., 35° C., 40° C., 45° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 75° C., 80° C., 85° C., 90° C., or 95° C. In some embodiments, the reduction incubation time is performed at a room temperature.

In some embodiments, the reduction technique comprises a reduction incubation time of about 30 minutes to about 70 minutes, such as about any of 35 minutes, 40 minutes, 45 minutes, 50 minutes, 55 minutes, 60 minutes, or 65 minutes, wherein the reduction incubation time is performed at a temperature of about 50° C. to about 70° C., such about any of 55° C., 60° C., or 65° C. In some embodiments, the reduction technique comprises use of an amount (as assessed based on the final concentration in the sample containing solution) of a reducing agent, e.g., DTT, of about 5 mM to about 25 mM, such as any of about 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, or 24 mM, and a reduction incubation time of about 30 minutes to about 70 minutes, such as about any of 35 minutes, 40 minutes, 45 minutes, 50 minutes, 55 minutes, 60 minutes, or 65 minutes, wherein the reduction incubation time is performed at a temperature of about 50° C. to about 70° C., such about any of 55° C., 60° C., or 65° C.

The reduction incubation temperature can be controlled by numerous techniques and combinations thereof. In some embodiments, the reduction incubation temperature is controlled by an ambient temperature, such as room temperature. In some embodiments, the reduction incubation temperature is controlled by a water bath. In some embodiments, the reduction incubation temperature is controlled by a heat block. In some embodiments, the reduction incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the reduction incubation time temperature are controlled by a technique described herein, such as cooling at room temperature or a ramp rate.

In some embodiments, the reduction technique comprises subjecting a sample, or a derivative thereof, e.g., a denatured sample, to a thermal cycle. In some embodiments, the thermal cycle comprises subjecting the sample, or a derivative thereof, to one or more of: (a) a block starting temperature (b) block set temperature (the temperature for the reduction incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 20° C. to about 100° C., such as any of 40° C. to about 80° C., about 50° C. to about 70° C., about 50° C. to about 60° C., about 55° C. to about 65° C., or about 60° C. to about 70° C., including about any of 50° C., 55° C., 60° C., 65° C., or 70° C. In some embodiments, the thermal cycle comprises: (a) a set block temperature of about 20° C. to about 100° C., such as any of about 40° C. to about 80° C., about 50° C. to about 70° C., about 50° C. to about 60° C., about 55° C. to about 65° C., or about 60° C. to about 70° C., including about any of 50° C., 55° C., 60° C., 65° C., or 70° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 60° C., such as any of about 15° C. to about 50° C., about 20° C. to about 40° C., about 20° C. to about 30° C., or about 20° C. to about 25° C., (b) a set block temperature of about 20° C. to about 100° C., such as any of about 40° C. to about 80° C., about 50° C. to about 70° C., about 50° C. to about 60° C., about 55° C. to about 65° C., or about 60° C. to about 70° C., including about any of 50° C., 55° C., 60° C., 65° C., or 70° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

In some embodiments, the reduction technique described herein is completed simultaneously with a thermal denaturation step. For example, the combined thermal denaturation technique and reduction technique comprises adding a reducing agent to a sample, or a derivative thereof, and then subjecting the sample, or the derivative thereof, to a temperature of about 60° C. to about 100° C., such about any of 60° C., 65° C., 70° C., 75° C., 80° C., 85° C., 90° C., 91° C., 92° C., 93° C., 94° C., 95° C., 96° C., 97° C., 98° C., 99° C., or 100° C., for an incubation time of at least about 1 minute, such as at least about any of 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes, or 60 minutes. In some embodiments, the combined thermal denaturation technique and reduction technique comprises adding a reducing agent to a sample, or a derivative thereof, and then subjecting the sample, or the derivative thereof, to a temperature of about 90° C. to about 100° C., for an incubation time of about 40 minutes to about 60 minutes, including 50 minutes.

### III. Alkylation Techniques

In certain aspects, the methods provided herein comprise performing an alkylation technique. In some embodiments, the alkylation technique is performed on a sample, or a derivative thereof, following the performance of a reduction technique. Alkylation techniques, generally speaking, prevent the reformation of one or more disulfide linkages between, e.g., cysteine residues of one or more polypeptides. This is done by, e.g., the addition of an acetamide moiety to the sulfur of a cysteine residue thereby producing an alkylated polypeptide. Alkylation techniques may reduce the presence of polypeptide conformations that inhibit or prevent protease cleavage of the one or more polypeptides. Alkylation techniques described herein comprise subjecting a sample, or a derivative thereof (e.g., a reduced sample), to an amount of an alkylating agent and incubating for an alkylation incubation time performed at a temperature or range thereof. In some embodiments, the method comprises subjecting a denatured sample to a reduction technique followed by an alkylation technique prior to performing a proteolytic digestion technique.

In some embodiments, the alkylating agent is iodoacetamide (IAA), 2-chloroacetamide, an acetamide salt, or any mixture thereof.

In some embodiments, the amount (as assessed based on the final concentration in the sample containing solution) of an alkylating agent, e.g., IAA, used in an alkylation technique is about 10 mM to about 100 mM, such as any of about 10 mM to about 50 mM, about 20 mM to about 40 mM, about 20 mM to about 36 mM, about 15 mM to about 25 mM, about 20 mM to about 25 mM, about 20 mM to about 80 mM, about 30 mM to about 70 mM, or about 40 mM to about 60 mM. In some embodiments, the amount of an alkylating agent used in an alkylation technique is at least about 10 mM, such as at least about any of 15 mM, 16 mM, 17 mM, 18 mM, 19 mM, 20 mM, 21 mM, 22 mM, 23 mM, 24 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some embodiments, the amount of an alkylating agent used in an alkylation technique is about 100 mM or less, such as about any of 95 mM or less, 90 mM or less, 85 mM or less, 80 mM or less, 75 mM or less, 70 mM or less, 65 mM or less, 60 mM or less, 55 mM or less, 50 mM or less, 45 mM or less, 40 mM or less, 35 mM or less, 30 mM or less, 25 mM or less, 24 mM or less, 23 mM or less, 22 mM or less, 21 mM or less, 20 mM or less, 19 mM or less, 18 mM or less, 17 mM or less, 16 mM or less, 15 mM or less, or 10 mM or less. In some embodiments, the amount of an alkylating agent used in an alkylation technique is about any of 10 mM, 15 mM, 20 mM, 20.5 mM, 21 mM, 21.5 mM, 22 mM, 22.5 mM, 23 mM, 23.5 mM, 24 mM, 24.5 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In some embodiments, the alkylation incubation time is about 5 minutes to about 60 minutes, such as any of about 10 minutes to about 50 minutes, about 20 minutes to about 40 minutes, about 25 minutes to about 35 minutes. In some embodiments, the alkylation incubation time is at least about 5 minutes, such as at least about any of 10 minutes, 15 minutes, 20 minutes, 25 minutes, 30 minutes, 35 minutes, 40 minutes, 45 minutes, 50 minutes, 55 minutes, or 60 minutes. In some embodiments, the alkylation incubation time is about 60 minutes or less, such as about any of 55 minutes or less, 50 minutes or less, 45 minutes or less, 40 minutes or less, 35 minutes or less, 30 minutes or less, 25 minutes or less, 20 minutes or less, 15 minutes or less, 10 minutes or less, or 5 minutes or less. In some embodiments, the alkylation incubation time is about any of 5 minutes, 10 minutes, 15 minutes, 20 minutes, 25 minutes, 30 minutes, 35 minutes, 40 minutes, 45 minutes, 50 minutes, 55 minutes, or 60 minutes.

In some embodiments, the alkylation incubation time is performed at a temperature of about 15° C. to about 100° C., such as any of about 15° C. to about 80° C., about 15° C. to about 60° C., about 15° C. to about 35° C., about 20° C. to about 30° C., or about 20° C. to about 25° C. In some embodiments, the alkylation incubation time is performed at a temperature of at least about 15° C., such as at least about any of 20° C., 21° C., 22° C., 23° C., 24° C., 25° C., 30° C., 35° C., 40° C., 45° C., 50° C., 55° C., 60° C., 65° C., 70° C., 75° C., 80° C., 85° C., 90° C., or 95° C. In some embodiments, the alkylation incubation time is performed at a temperature of about 95° C. or less, such as about any of 90° C. or less, 85° C. or less, 80° C. or less, 75° C. or less, 70° C. or less, 65° C. or less, 60° C. or less, 55° C. or less, 50° C. or less, 45° C. or less, 40° C. or less, 35° C. or less, 30° C. or less, 25° C. or less, 24° C. or less, 23° C. or less, 22° C. or less, 21° C. or less, or 20° C. or less. In some embodiments, the alkylation incubation time is performed at a temperature of about any of 15° C., 16° C., 17° C., 18° C., 19° C., 20° C., 21° C., 22° C., 23° C., 24° C., 25° C., 30° C., 35° C., 40° C., 45° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 75° C., 80° C., 85° C., 90° C., or 95° C. In some embodiments, the alkylation incubation time is performed at a room temperature.

In some embodiments, the alkylation technique comprises an alkylation incubation time of about 5 minutes to about 60 minutes, such as about any of 15 minutes, 20 minutes, 25 minutes, 30 minutes, 35 minutes, or 40 minutes, wherein the alkylation incubation time is performed at a temperature of about 15° C. to about 30° C., such about any of 20° C., 21° C., 22° C., 23° C., 24° C., 25° C. In some embodiments, the alkylation technique comprises use of an amount (containing solution based on the final concentration in the sample) of an alkylating agent, e.g., IAA, of about 15 mM to about 40 mM, such as any of about 20 mM, 20.5 mM, 21 mM, 21.5 mM, 22 mM, 22.5 mM, 23 mM, 23.5 mM, 24 mM, 24.5 mM, 25 mM, 25.5 mM, 26 mM, 26.5 mM, 27 mM, 27.5 mM, 28 mM, 28.5 mM, 29 mM, 29.5 mM, 30 mM, 30.5 mM, 31 mM, 31.5 mM, 32 mM, 32.5 mM, 33 mM, 33.5 mM, 34 mM, 34.5 mM, 35 mM, 35.5 mM, 36 mM, 36.5 mM, 37 mM, 37.5 mM, 38 mM, and an alkylation incubation time of about 5 minutes to about 60 minutes, such as about any of 15 minutes, 20 minutes, 25 minutes, 30 minutes, 35 minutes, or 40 minutes, wherein the alkylation incubation time is performed at a temperature of about 15° C. to about 30° C., such about any of 20° C., 21° C., 22° C., 23° C., 24° C., or 25° C.

The alkylation incubation temperature can be controlled by numerous techniques and combinations thereof. In some embodiments, the alkylation incubation temperature is controlled by an ambient temperature, such as room temperature. In some embodiments, the alkylation incubation temperature is controlled by a water bath. In some embodiments, the alkylation incubation temperature is controlled by a heat block. In some embodiments, the alkylation incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the alkylation incubation time temperature are controlled by a technique described herein, such as cooling at room temperature or a ramp rate.

In some embodiments, the alkylation technique comprises subjecting a sample, or a derivative thereof, e.g., a denatured sample, to a thermal cycle. In some embodiments, the thermal cycle comprises subjecting the sample, or a derivative thereof, to one or more of: (a) a block starting temperature (b) block set temperature (the temperature for the alkylation incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., or 25° C. In some embodiments, the thermal cycle comprises: (a) a set block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., 25° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., 25° C., (b) a set block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., 25° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C.

In some embodiments, the alkylation technique further comprises quenching the alkylating agent comprising use of a neutralizing agent. In some embodiments, the neutralizing agent is a reducing agent. In some embodiments, the reducing agent is dithiothreitol (DTT), tris(2-carboxyethyl) phosphine (TCEP), beta-mercaptoethanol (BME), or a cysteine, or any mixture thereof. In some embodiments, the neutralizing agent is added in an amount to fully quench the amount of the alkylating agent, such as in an amount greater than or equal to a molar amount of an active moiety of the alkylating agent. In some embodiments, the amount (as assessed based on the final concentration in the sample containing solution) of the neutralizing agent is about 1 mM to about 100 mM.

In some embodiments, the alkylation technique, in whole or in part, is performed substantially in a low light condition. In some embodiments, the alkylation incubation time is performed in a low light condition. In some embodiments, the low light condition is in the dark or a location substantially devoid of sunlight and/or room lighting, such as in a desk drawer. In some embodiments, the low light condition is a filtered light, such as red light.

In some embodiments, the alkylating agent is sourced from a stock solution. In some embodiments, the stock solution is prepared within about 1 hour, such as within about any of 50 minutes, 40 minutes, 30 minutes, 20 minutes, or 10 minutes, of use.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

### IV. Proteolytic Digestion Techniques

In certain aspects, the methods provided herein comprise performing a proteolytic digestion technique. In some embodiments, the proteolytic digestion technique is performed on a sample, or a derivative thereof, following thermal denaturation and/or any additional steps intended to expose protease cleavage sites. Proteolytic digestion techniques, generally speaking, cleave polypeptides at known cleavage sites. For example, trypsin is a serine protease that generally cleaves polypeptides at the carboxyl side (C-terminal side) of lysine and arginine residues. Certain exceptions apply to the cleavage pattern of trypsin, such as due to proximity of a proline residue and/or a post-translational modification causing steric hindrance relative to the cleavage site. Proteolytic digestion techniques described herein comprise subjecting a sample, or a derivative thereof (e.g., a denatured sample or an alkylated sample, including an alkylated sample subjected to a reduction technique prior to an alkylation technique), to an amount of one or more proteases and incubating for a digestion incubation time performed at a temperature or range thereof.

In some embodiments, each of the one or more proteases is trypsin, LysC, LysN, AspN, GluC, ArgC, IdeS, IdeZ, PNGase F, thermolysin, pepsin, elastase, TEV, or Factor Xa, or any mixture thereof. In some embodiments, wherein two or more proteases are used, the weight ratio between a first protease and a second protease is about 1:10 to about 10:1, such as about any of about 1:9, 1:8, 1:7: 1:6, 1:5, 1:4, 1:3, 1:2, or 1:1. In some embodiments, the one or more proteases is trypsin. In some embodiments, the one or more proteases is a mixture of trypsin and LysC, such as in a weight ratio of about 1:1. In some embodiments, the one or more proteases is selected based on the type and/or characteristic of a biological sample used in the methods herein. In some embodiments, the biological sample is a plasma sample, wherein the one or more proteases is trypsin and Lys-C, such as in a weight ratio of about 1:1. In some embodiments, the biological sample is a serum sample, wherein the one or more proteases is trypsin. In some embodiments, the protease is a modified protease, such as comprising a modification to prevent or inhibit self-proteolysis. In some embodiments, the modified protease is a modified trypsin, such as a methylated and/or an acetylated trypsin. In some embodiments, the modified trypsin is a tosyl phenylalanyl chloromethyl ketone (TPCK)-treated trypsin.

In some embodiments, the amount of a protease, e.g., trypsin or LysC, used in a proteolytic digestion technique is based on a weight ratio relative to the polypeptide content of a sample, or a derivative thereof, (i.e., weight of a protease: weight of polypeptide content) of about 1:200 to about 1:10, such as any of about 1:100 to about 1:10, about 1:50 to about 1:10, about 1:40 to about 1:20, about 1:50 to about 1:30, about 1:45 to about 1:35, about 1:20 to about 1:40, about 1:30 to about 1:10, or about 1:25 to about 1:15. In some embodiments, the amount of a protease used in a proteolytic digestion technique is at least about 1:200, such as at least about any of 1:190, 1:180, 1:170, 1:160, 1:150, 1:140, 1:130, 1:120, 1:110, 1:100, 1:95, 1:90, 1:85, 1:80, 1:75, 1:70, 1:65, 1:60, 1:55, 1:50, 1:45, 1:40, 1:35, 1:30, 1:25, 1:20, 1:15, or 1:10. In some embodiments, the amount of a protease used in a proteolytic digestion technique is about 1:10 or less, such as about any of 1:15 or less, 1:20 or less, 1:25 or less, 1:30 or less, 1:35 or less, 1:40 or less, 1:45 or less, 1:50 or less, 1:55 or less, 1:60 or less, 1:65 or less, 1:70 or less, 1:75 or less, 1:80 or less, 1:85 or less, 1:90 or less, 1:95 or less, 1:100 or less, 1:110 or less, 1:120 or less, 1:130 or less, 1:140 or less, 1:150 or less, 1:160 or less, 1:170 or less, 1:180 or less, 1:190 or less, or 1:200 or less. In some embodiments, the amount of a protease used in a proteolytic digestion technique is about any of 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, 1:110, 1:120, 1:130, 1:140, 1:150, 1:160, 1:170, 1:180, 1:190, or 1:200. In some embodiments, the proteolytic digestion technique comprises the use of two or more proteases, such as a combination of trypsin and LysC, and in such embodiments, the amount of each protease (such as described above) can be summed to a total amount of proteases used in a proteolytic digestion technique.

In some embodiments, the proteolytic digestion incubation time is about 20 minutes to about 36 hours, such as any of about 1 hour to about 18 hours, about 5 hours to about 24 hours, about 12 hours to about 24 hours, about 16 hours to about 20 hours, or about 12 hours to about 36 hours. In some embodiments, the proteolytic digestion incubation time is about 36 hours or less, such as about any of 32 hours or less, 30 hours or less, 28 hours or less, 26 hours or less, 24 hours or less, 22 hours or less, 20 hours or less, 19 hours or less, 18 hours or less, 17 hours or less, 16 hours or less, 15 hours or less, 14 hours or less, 13 hours or less, 12 hours or less, 11 hours or less, 10 hours or less, 9 hours or less, 8 hours or less, 7 hours or less, 6 hours or less, 5 hours or less, 4 hours or less, 3 hours or less, 2 hours or less, or 1 hours or less. In some embodiments, the proteolytic digestion incubation time is at least about 20 minutes, such as at least about any of 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 22 hours, 24 hours, 26 hours, 28 hours, 30 hours, 32 hours, 34 hours, or 36 hours. In some embodiments, the proteolytic digestion incubation time is about any of 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 22 hours, 24 hours, 26 hours, 28 hours, 30 hours, 32 hours, 34 hours, or 36 hours.

In some embodiments, the digestion incubation time is performed at a temperature of about 20° C. to about 60° C., such as any of about 20° C. to about 25° C., about 20° C. to about 30° C., about 25° C. to about 40° C., about 35° C. to about 40° C., or about 35° C. to about 50° C. In some embodiments, the digestion incubation time is performed at a temperature of at least about 20° C., such as at least about any of 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 42° C., 44° C., 46° C., 48° C., 50° C., 52° C., 54° C., 56° C., 58° C., or 60° C. In some embodiments, the digestion incubation time is performed at a temperature of about 60° C. or less, such about any of 58° C. or less, 56° C. or less, 54° C. or less, 52° C. or less, 50° C. or less, 48° C. or less, 46° C. or less, 44° C. or less, 42° C. or less, 40° C. or less, 39° C. or less, 38° C. or less, 37° C. or less, 36° C. or less, 35° C. or less, 34° C. or less, 33° C. or less, 32° C. or less, 31° C. or less, 30° C. or less, 29° C. or less, 28° C. or less, 27° C. or less, 26° C. or less, 25° C. or less, 24° C. or less, 23° C. or less, 22° C. or less, 21° C. or less, or 20° C. or less. In some embodiments, the digestion incubation time is performed at a temperature of about any of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 42° C., 44° C., 46° C., 48° C., 50° C., 52° C., 54° C., 56° C., 58° C., or 60° C. In some embodiments, the reduction incubation time is performed at a room temperature.

In some embodiments, the proteolytic digestion technique comprises a digestion incubation time of about 12 hours to about 24 hours, such as about any of 13 hours, 14 hours, 15 hours, 16 hours, 17 hours, 18 hours, 19 hours, 20 hours, 21 hours, 22 hours, 23 hours, wherein the digestion incubation time is performed at a temperature of about 20° C. to about 40° C., such about any of 22° C., 24° C., 26° C., 28° C., 30° C., 31° C., 32° C., 33° C., 34° C., 35° C., 36° C., 37° C., 38° C., 39° C. In some embodiments, the proteolytic digestion technique comprises use of an amount a protease for each of one or more proteases, e.g., trypsin and/or LysC, of about 1:15 to about 1:45, such as about any of 1:20, 1:25, 1:30, 1:35, or 1:40 (as measured based on the amount of the protease to the amount of polypeptide in a sample or a derivative thereof), and a digestion incubation time of about 12 hours to about 24 hours, such as about any of 13 hours, 14 hours, 15 hours, 16 hours, 17 hours, 18 hours, 19 hours, 20 hours, 21 hours, 22 hours, 23 hours, wherein the digestion incubation time is performed at a temperature of about 20° C. to about 40° C., such about any of 22° C., 24° C., 26° C., 28° C., 30° C., 31° C., 32° C., 33° C., 34° C., 35° C., 36° C., 37° C., 38° C., 39° C. In some embodiments, the proteolytic digestion technique is performed on a plasma sample or a derivate thereof, wherein the proteolytic digestion technique comprises a 1:40 ratio of trypsin to polypeptide in the sample of the derivative thereof and a 1:40 ratio of LysC to polypeptide in the sample or the derivative thereof. In some embodiments, the proteolytic digestion technique is performed on a serum sample or a derivate thereof, wherein the proteolytic digestion technique comprises a 1:20 ratio of trypsin.

The digestion incubation temperature can be controlled by numerous techniques and combinations thereof. In some embodiments, the digestion incubation temperature is controlled by an ambient temperature, such as room temperature. In some embodiments, the digestion incubation temperature is controlled by a water bath. In some embodiments, the digestion incubation temperature is controlled by a heat block. In some embodiments, the digestion incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the digestion incubation time temperature are controlled by a technique described herein, such as cooling at room temperature or a ramp rate.

In some embodiments, the proteolytic digestion technique comprises subjecting a sample, or a derivative thereof, e.g., an alkylated sample (including an alkylated sample quenched with a neutralizing agent), to a thermal cycle. In some embodiments, the thermal cycle comprises subjecting the sample, or a derivative thereof, to one or more of: (a) a block starting temperature (b) block set temperature (the temperature for the digestion incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 20° C. to about 50° C., including about any of 22° C., 24° C., 26° C., 28° C., 30° C., 31° C., 32° C., 33° C., 34° C., 35° C., 36° C., 37° C., 38° C., 39° C., 40° C., 42° C., 44° C., 46° C., 48° C.

In some embodiments, the thermal cycle comprises: (a) a set block temperature of about 20° C. to about 50° C., including about any of 22° C., 24° C., 26° C., 28° C., 30° C., 31° C., 32° C., 33° C., 34° C., 35° C., 36° C., 37° C., 38° C., 39° C., 40° C., 42° C., 44° C., 46° C., 48° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 35° C., such as any of about 20° C. to about 35° C., or about 20° C. to about 25° C., (b) a set block temperature of about 20° C. to about 50° C., including about any of 22° C., 24° C., 26° C., 28° C., 30° C., 31° C., 32° C., 33° C., 34° C., 35° C., 36° C., 37° C., 38° C., 39° C., 40° C., 42° C., 44° C., 46° C., 48° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

In some embodiments, the proteolytic digestion technique further comprises quenching the one or more proteolytic enzymes. In some embodiments, quenching the one or more proteolytic enzymes comprises denaturing the one or more proteolytic enzymes. In some embodiments, quenching the one or more proteolytic enzymes comprise adding an amount of an acid. In some embodiments, the acid is formic acid (FA) or trifluoroacetic acid (TFA), or a mixture thereof. In some embodiments, the amount (as assessed based on the final concentration in the sample containing solution) of the acid added is about any of 0.1% v/v, 0.2% v/v, 0.3% v/v, 0.4% v/v, 0.5% v/v, 0.6% v/v, 0.7% v/v, 0.8% v/v, 0.9% v/v, 1% v/v, 1.1% v/v, 1.2% v/v, 1.3% v/v, 1.4% v/v, 1.5% v/v, 1.6% v/v, 1.7% v/v, 1.8% v/v, 1.9% v/v, or 2% v/v.

### V. Additional Techniques

In certain aspects, the method provided herein comprise subjecting the proteolytic digest sample comprising a proteolytic glycopeptide to one or more additional steps prior to subjecting the proteolytic digested sample, or a derivative thereof, to a liquid chromatography-mass spectrometry (LC-MS) technique using a liquid chromatography system and a mass spectrometer. In some embodiments, the LC system is online with the MS (i.e., eluate from the LC system is directly introduced to the MS).

In some embodiments, the method further comprises adding a standard to the proteolytic digest sample prior to the LC-MS technique. In some embodiments, the standard is a stable isotope-internal standard (SI-IS) peptide mixture.

In some embodiments, the biological sample is not subjected to a high-abundant protein depletion technique prior to the thermal denaturation technique. For example, in some embodiments, the high-abundant protein depletion technique removes highly abundant proteins present in a blood sample, such as serum albumin.

### E5. Methods for Performing a LC-MS Analysis of a Proteolytic Glycopeptide

In certain aspects, provided herein is a method for performing a LC-MS analysis on a sample comprising a proteolytic glycopeptide. In some embodiments, the liquid chromatography (LC) system is online with a mass spectrometer (i.e., proteolytic peptide species, including glycopeptides, are eluted from the LC system directing into the mass spectrometer via a mass spectrometer interface. In some embodiments, the LC technique comprises performing a chromatographic separation of one or more proteolytic peptides, including glycopeptides. In some embodiments, the one or more proteolytic peptides subjected to a chromatographic separation are obtained from a proteolytic digest sample, such as described herein.

In some embodiments, the chromatographic separation comprises a gradient separation performing using mixtures of an aqueous mobile phase and an organic mobile phase. In some embodiments, the chromatographic separation comprises isocratic period, such as a period of at least about 90%, such as at least about any of 91%, 92%, 93%, 94%, 95%, 96%, 97%, 98%, 99%, or 100%, of an aqueous mobile phase to produce the initial eluate that is diverted from the mass spectrometer interface.

In some embodiments, the LC system comprises a reversed-phase chromatography column. In some embodiments, the reversed-phase column comprises an alkyl moiety, such as C18.

The present application contemplates a diverse array of additional features of LC-MS techniques for analyzing a sample comprising a glycopeptide using a mass spectrometer. In some embodiments, the liquid chromatography system comprises a high performance liquid chromatography system. In some embodiments, the liquid chromatography system comprises an ultra-high performance liquid chromatography system. In some embodiments, the liquid chromatography system comprises a high-flow liquid chromatography system. In some embodiments, the liquid chromatography system comprises a low-flow liquid chromatography system, such as a micro-flow liquid chromatography system or a nano-flow liquid chromatography system. In some embodiments, the liquid chromatography system is coupled, such as directly interfaced, with a mass spectrometer.

In some embodiment, the mass spectrometry technique comprises an ionization technique. Ionization techniques contemplated by the present application include techniques capable of charging polypeptides and peptide products, including glycopeptides. Thus, in some embodiments, the ionization technique is electrospray ionization. In some embodiments, the ionization technique is nano-electrospray ionization. In some embodiments, the ionization technique is atmospheric pressure chemical ionization. In some embodiments, the ionization technique is atmospheric pressure photoionization.

A diverse array of mass spectrometers are contemplated as compatible with the description, include high-resolution mass spectrometers and low-resolution mass spectrometers. In some embodiments, the mass spectrometer is a time-of-flight (TOF) mass spectrometer. In some embodiments, the mass spectrometer is a quadrupole time-of-flight (Q-TOF) mass spectrometer. In some embodiments, the mass spectrometer is a quadrupole ion trap time-of-flight (QIT-TOF) mass spectrometer. In some embodiments, the mass spectrometer is an ion trap. In some embodiments, the mass spectrometer is a single quadrupole. In some embodiments, the mass spectrometer is a triple quadrupole (QQQ). In some embodiments, the mass spectrometer is an orbitrap. In some embodiments, the mass spectrometer is a quadrupole orbitrap. In some embodiments, the mass spectrometer is a fourier transform ion cyclotron resonance (FT) mass spectrometer. In some embodiments, the mass spectrometer is a quadrupole fourier transform ion cyclotron resonance (Q-FT) mass spectrometer. In some embodiments, the mass spectrometry technique comprises positive ion mode. In some embodiments, the mass spectrometry technique comprises negative ion mode. In some embodiments, the mass spectrometry technique comprises an ion mobility mass spectrometry technique.

In some embodiments, the LC-MS technique comprises processing obtained signals MS from the mass spectrometer. In some embodiments, the LC-MS technique comprises peak detection. In some embodiments, the LC-MS technique comprises determining ionization intensity of an ionized peptide product. In some embodiments, the LC-MS technique comprises determining peak height of an ionized peptide product. In some embodiments, the LC-MS technique comprises determining peak area of an ionized peptide product. In some embodiments, the LC-MS technique comprises determining peak volume of an ionized peptide product. In some embodiments, the LC-MS technique comprises identifying an ionized peptide product by amino acid sequence. In some embodiments, the LC-MS technique comprises determining the site of a post-translational modification of an ionized peptide, such as the site of a glycosylation. In some embodiments, the LC-MS technique comprises determining the glycan structure, or a characteristic thereof, of an ionized peptide product. In some embodiments, the LC-MS technique comprises manually validating the ionized peptide product acid sequence assignments. In some embodiments, the LC-MS technique comprises a quantification technique.

### F5. Exemplary Methods

In certain aspects, provided is a method for processing a proteolytic digest sample for use in a LC-MS analysis, wherein the proteolytic digest sample comprises a plurality of proteolytically digested peptides comprising at least one proteolytically digested glycopeptide, the method comprising: (A) loading a HILIC load derived from the proteolytic digest sample to a solid phase extraction column comprising a HILIC medium to associate the at least one proteolytically digested glycopeptide with the HILIC medium, wherein the loading of the HILIC load to the solid phase extraction column is initiated when the HILIC medium is in a dry state; and (B) subjecting the HILIC medium to an elution solution to obtain a HILIC eluate comprising the at least one proteolytically digested glycopeptide. In some embodiments, the dry state of a HILIC medium is characterized by the HILIC medium comprising less than about 5% (v/v), such as less than about any of 4.5% (v/v), 4% (v/v), 3.5% (v/v), 3% (v/v), 2.5% (v/v), 2% (v/v), 1.5% (v/v), 1% (v/v), or 0.5% (v/v), of a liquid at the initiation of the loading of a HILIC load to a solid phase extraction column comprising the HILIC medium. In some embodiments, the HILIC load comprises about 300 μg of the plurality of proteolytically digested peptides. In some embodiments, the HILIC load comprises about 600 μg of the plurality of proteolytically digested peptides. In some embodiments, the HILIC load comprises at least about 80% (v/v) ACN. In some embodiments the HILIC load is 220 μl or less. In some embodiments, the method further comprises performing a wash step following the loading and prior to subjecting the HILIC medium to an elution liquid. In some embodiments, the solid phase extraction column comprises about 2.5 mg to about 3.5 mg, including about 3 mg, of a HILIC medium in a dry state, a bed volume of the HILIC medium in a dry state of about 4 μl to about 6 μl, including about 5 μl, and wherein the HILIC medium comprises silica particles covalently bonded with carbamoyl groups. In some embodiments, the solid phase extraction column is a GlykoPrep Cleanup cartridge (e.g., Product code: GS96-CU; WS0263).

In certain aspects, provided is a method for processing a proteolytic digest sample for use in a LC-MS analysis, wherein the proteolytic digest sample comprises a plurality of proteolytically digested peptides comprising at least one proteolytically digested glycopeptide, the method comprising: (A) loading a HILIC load derived from the proteolytic digest sample to a solid phase extraction column comprising a HILIC medium to associate the at least one proteolytically digested glycopeptide with the HILIC medium, wherein the HILIC load loaded to the solid phase extraction column has an amount of the plurality of proteolytically digested peptides characterized by one or both of: (i) a ratio of the weight of the plurality of proteolytically digested peptides over the weight of the HILIC medium in the dry state of at least about 0.05; and/or (ii) a ratio of the weight of the plurality of proteolytically digested peptides relative to the bed volume of the HILIC medium in the dry state of at least about 30 μg/μl; and (B) subjecting the HILIC medium to an elution solution to obtain a HILIC eluate comprising the at least one proteolytically digested glycopeptide. In some embodiments, the dry state of a HILIC medium is characterized by the HILIC medium comprising less than about 5% (v/v), such as less than about any of 4.5% (v/v), 4% (v/v), 3.5% (v/v), 3% (v/v), 2.5% (v/v), 2% (v/v), 1.5% (v/v), 1% (v/v), or 0.5% (v/v), of a liquid at the initiation of the loading of a HILIC load to a solid phase extraction column comprising the HILIC medium. In some embodiments, the HILIC load comprises about 300 μg of the plurality of proteolytically digested peptides. In some embodiments, the HILIC load comprises about 600 μg of the plurality of proteolytically digested peptides. In some embodiments, the HILIC load comprises at least about 80% (v/v) ACN. In some embodiments the HILIC load is 220 μl or less. In some embodiments, the method further comprises performing a wash step following the loading and prior to subjecting the HILIC medium to an elution liquid. In some embodiments, the solid phase extraction column comprises about 2.5 mg to about 3.5 mg, including about 3 mg, of a HILIC medium in a dry state, a bed volume of the HILIC medium in a dry state of about 4 μl to about 6 μl, including about 5 μl, and wherein the HILIC medium comprises silica particles covalently bonded with carbamoyl groups. In some embodiments, the solid phase extraction column is a GlykoPrep Cleanup cartridge (e.g., Product code: GS96-CU; WS0263).

In certain aspects, provided is a method for processing a proteolytic digest sample for use in a LC-MS analysis, wherein the proteolytic digest sample comprises a plurality of proteolytically digested peptides comprising at least one proteolytically digested glycopeptide, the method comprising: (A) loading a HILIC load derived from the proteolytic digest sample to a solid phase extraction column comprising a HILIC medium to associate the at least one proteolytically digested glycopeptide with the HILIC medium, the HILIC load loaded to the solid phase extraction column has a concentration of an organic solvent of at least about 70% (v/v); and (B) subjecting the HILIC medium to an elution solution to obtain a HILIC eluate comprising the at least one proteolytically digested glycopeptide. In some embodiments, the HILIC load comprises a concentration of an organic solvent of at least about 80% (v/v), such as at least about any of 85% (v/v), 90% (v/v), or 95% (v/v). In some embodiments, the organic solvent is ACN. In some embodiments, the HILIC load comprises about 300 μg of the plurality of proteolytically digested peptides. In some embodiments, the HILIC load comprises about 600 μg of the plurality of proteolytically digested peptides. In some embodiments, the HILIC load comprises at least about 80% (v/v) ACN. In some embodiments the HILIC load is 220 μl or less. In some embodiments, the method further comprises performing a wash step following the loading and prior to subjecting the HILIC medium to an elution liquid. In some embodiments, the solid phase extraction column comprises about 2.5 mg to about 3.5 mg, including about 3 mg, of a HILIC medium in a dry state, a bed volume of the HILIC medium in a dry state of about 4 μl to about 6 μl, including about 5 μl, and wherein the HILIC medium comprises silica particles covalently bonded with carbamoyl groups. In some embodiments, the solid phase extraction column is a GlykoPrep Cleanup cartridge (e.g., Product code: GS96-CU; WS0263).

In certain aspects, provided is a method for processing a proteolytic digest sample for use in a LC-MS analysis, wherein the proteolytic digest sample comprises a plurality of proteolytically digested peptides comprising at least one proteolytically digested glycopeptide, the method comprising: (A) loading a HILIC load derived from the proteolytic digest sample to a solid phase extraction column comprising a HILIC medium to associate the at least one proteolytically digested glycopeptide with the HILIC medium, wherein (1) the loading of the HILIC load to the solid phase extraction column is initiated when the HILIC medium is in a dry state; and (2) wherein the HILIC load loaded to the solid phase extraction column has an amount of the plurality of proteolytically digested peptides characterized by one or both of: (i) a ratio of the weight of the plurality of proteolytically digested peptides over the weight of the HILIC medium in the dry state of at least about 0.05; and/or (ii) a ratio of the weight of the plurality of proteolytically digested peptides relative to the bed volume of the HILIC medium in the dry state of at least about 30 μg/μl; and (B) subjecting the HILIC medium to an elution solution to obtain a HILIC eluate comprising the at least one proteolytically digested glycopeptide. In some embodiments, the dry state of a HILIC medium is characterized by the HILIC medium comprising less than about 5% (v/v), such as less than about any of 4.5% (v/v), 4% (v/v), 3.5% (v/v), 3% (v/v), 2.5% (v/v), 2% (v/v), 1.5% (v/v), 1% (v/v), or 0.5% (v/v), of a liquid at the initiation of the loading of a HILIC load to a solid phase extraction column comprising the HILIC medium. In some embodiments, the HILIC load comprises about 300 μg of the plurality of proteolytically digested peptides. In some embodiments, the HILIC load comprises about 600 μg of the plurality of proteolytically digested peptides. In some embodiments, the HILIC load comprises at least about 80% (v/v) ACN. In some embodiments the HILIC load is 220 μl or less. In some embodiments, the method further comprises performing a wash step following the loading and prior to subjecting the HILIC medium to an elution liquid. In some embodiments, the solid phase extraction column comprises about 2.5 mg to about 3.5 mg, including about 3 mg, of a HILIC medium in a dry state, a bed volume of the HILIC medium in a dry state of about 4 μl to about 6 μl, including about 5 μl, and wherein the HILIC medium comprises silica particles covalently bonded with carbamoyl groups. In some embodiments, the solid phase extraction column is a GlykoPrep Cleanup cartridge (e.g., Product code: GS96-CU; WS0263).

In certain aspects, provided is a method for processing a proteolytic digest sample for use in a liquid chromatography-mass spectrometry (LC-MS) analysis, wherein the proteolytic digest sample comprises a plurality of proteolytically digested peptides comprising at least one proteolytically digested glycopeptide, the method comprising: (A) loading a hydrophilic interaction liquid chromatography (HILIC) load derived from the proteolytic digest sample to a solid phase extraction column comprising a HILIC medium to associate the at least one proteolytically digested glycopeptide with the HILIC medium, wherein the loading of the HILIC load to the solid phase extraction column is initiated when the HILIC medium is in a dry state, and wherein the HILIC load loaded to the solid phase extraction column has a concentration of an organic solvent of at least about 70% (v/v); and (B) subjecting the HILIC medium to an elution solution to obtain a HILIC eluate comprising the at least one proteolytically digested glycopeptide. In some embodiments, the dry state of a HILIC medium is characterized by the HILIC medium comprising less than about 5% (v/v), such as less than about any of 4.5% (v/v), 4% (v/v), 3.5% (v/v), 3% (v/v), 2.5% (v/v), 2% (v/v), 1.5% (v/v), 1% (v/v), or 0.5% (v/v), of a liquid at the initiation of the loading of a HILIC load to a solid phase extraction column comprising the HILIC medium. In some embodiments, the HILIC load comprises a concentration of an organic solvent of at least about 80% (v/v), such as at least about any of 85% (v/v), 90% (v/v), or 95% (v/v). In some embodiments, the organic solvent is ACN. In some embodiments, the HILIC load comprises about 300 μg of the plurality of proteolytically digested peptides. In some embodiments, the HILIC load comprises about 600 μg of the plurality of proteolytically digested peptides. In some embodiments, the HILIC load comprises at least about 80% (v/v) ACN. In some embodiments the HILIC load is 220 μl or less. In some embodiments, the method further comprises performing a wash step following the loading and prior to subjecting the HILIC medium to an elution liquid. In some embodiments, the solid phase extraction column comprises about 2.5 mg to about 3.5 mg, including about 3 mg, of a HILIC medium in a dry state, a bed volume of the HILIC medium in a dry state of about 4 μl to about 6 μl, including about 5 μl, and wherein the HILIC medium comprises silica particles covalently bonded with carbamoyl groups. In some embodiments, the solid phase extraction column is a GlykoPrep Cleanup cartridge (e.g., Product code: GS96-CU; WS0263).

In certain aspects, provided is a method for processing a proteolytic digest sample for use in a LC-MS analysis, wherein the proteolytic digest sample comprises a plurality of proteolytically digested peptides comprising at least one proteolytically digested glycopeptide, the method comprising: (A) loading a HILIC load derived from the proteolytic digest sample to a solid phase extraction column comprising a HILIC medium to associate the at least one proteolytically digested glycopeptide with the HILIC medium, wherein (1) the HILIC load loaded to the solid phase extraction column has an amount of the plurality of proteolytically digested peptides characterized by one or both of: (i) a ratio of the weight of the plurality of proteolytically digested peptides over the weight of the HILIC medium in the dry state of at least about 0.05; and/or (ii) a ratio of the weight of the plurality of proteolytically digested peptides relative to the bed volume of the HILIC medium in the dry state of at least about 30 μg/μl; and (2) the HILIC load loaded to the solid phase extraction column has a concentration of an organic solvent of at least about 70% (v/v); and (B) subjecting the HILIC medium to an elution solution to obtain a HILIC eluate comprising the at least one proteolytically digested glycopeptide. In some embodiments, the dry state of a HILIC medium is characterized by the HILIC medium comprising less than about 5% (v/v), such as less than about any of 4.5% (v/v), 4% (v/v), 3.5% (v/v), 3% (v/v), 2.5% (v/v), 2% (v/v), 1.5% (v/v), 1% (v/v), or 0.5% (v/v), of a liquid at the initiation of the loading of a HILIC load to a solid phase extraction column comprising the HILIC medium. In some embodiments, the HILIC load comprises about 300 μg of the plurality of proteolytically digested peptides. In some embodiments, the HILIC load comprises about 600 μg of the plurality of proteolytically digested peptides. In some embodiments, the HILIC load comprises at least about 80% (v/v) ACN. In some embodiments the HILIC load is 220 μl or less. In some embodiments, the method further comprises performing a wash step following the loading and prior to subjecting the HILIC medium to an elution liquid. In some embodiments, the solid phase extraction column comprises about 2.5 mg to about 3.5 mg, including about 3 mg, of a HILIC medium in a dry state, a bed volume of the HILIC medium in a dry state of about 4 μl to about 6 μl, including about 5 μl, and wherein the HILIC medium comprises silica particles covalently bonded with carbamoyl groups. In some embodiments, the solid phase extraction column is a GlykoPrep Cleanup cartridge (e.g., Product code: GS96-CU; WS0263).

In certain aspects, provided is a method for processing a proteolytic digest sample for use in a LC-MS analysis, wherein the proteolytic digest sample comprises a plurality of proteolytically digested peptides comprising at least one proteolytically digested glycopeptide, the method comprising: (A) loading a HILIC load derived from the proteolytic digest sample to a solid phase extraction column comprising a HILIC medium to associate the at least one proteolytically digested glycopeptide with the HILIC medium, wherein (1) the loading of the HILIC load to the solid phase extraction column is initiated when the HILIC medium is in a dry state; (2) the HILIC load loaded to the solid phase extraction column has an amount of the plurality of proteolytically digested peptides characterized by one or both of: (i) a ratio of the weight of the plurality of proteolytically digested peptides over the weight of the HILIC medium in the dry state of at least about 0.05; and/or (ii) a ratio of the weight of the plurality of proteolytically digested peptides relative to the bed volume of the HILIC medium in the dry state of at least about 30 μg/μl; and (3) the HILIC load loaded to the solid phase extraction column has a concentration of an organic solvent of at least about 70% (v/v); and (B) subjecting the HILIC medium to an elution solution to obtain a HILIC eluate comprising the at least one proteolytically digested glycopeptide. In some embodiments, the dry state of a HILIC medium is characterized by the HILIC medium comprising less than about 5% (v/v), such as less than about any of 4.5% (v/v), 4% (v/v), 3.5% (v/v), 3% (v/v), 2.5% (v/v), 2% (v/v), 1.5% (v/v), 1% (v/v), or 0.5% (v/v), of a liquid at the initiation of the loading of a HILIC load to a solid phase extraction column comprising the HILIC medium. In some embodiments, the HILIC load comprises about 300 μg of the plurality of proteolytically digested peptides. In some embodiments, the HILIC load comprises about 600 μg of the plurality of proteolytically digested peptides. In some embodiments, the HILIC load comprises at least about 80% (v/v) ACN. In some embodiments the HILIC load is 220 μl or less. In some embodiments, the method further comprises performing a wash step following the loading and prior to subjecting the HILIC medium to an elution liquid. In some embodiments, the solid phase extraction column comprises about 2.5 mg to about 3.5 mg, including about 3 mg, of a HILIC medium in a dry state, a bed volume of the HILIC medium in a dry state of about 4 μl to about 6 μl, including about 5 μl, and wherein the HILIC medium comprises silica particles covalently bonded with carbamoyl groups. In some embodiments, the solid phase extraction column is a GlykoPrep Cleanup cartridge (e.g., Product code: GS96-CU; WS0263).

### G5. Samples and Components Thereof

The methods provided herein are contemplated to be suitable for analyzing a diverse array of samples, such as biological samples. In some embodiments, the sample is a blood sample, such as a whole blood sample. In some embodiments, the sample is a plasma sample. In some embodiments, the sample is a serum sample. In some embodiments, the sample is a tissue sample. Plasma is a fluid component of blood that is obtained when a clotting-prevention agent is added to whole blood and then the tube is centrifuged to separate the cellular material. The upper lighter colored liquid layer in the tube is removed as plasma. Common anti-coagulant agents are EDTA (ethylenediaminetetraacetic acid), heparin, and citrate. Serum is a fluid obtained when whole blood is allowed to clot in a tube and then centrifuged so that the clotted blood, including red cells, are at the bottom of the collection tube, leaving a straw-colored liquid above the clot. The straw-colored liquid in the tube is removed as serum.

The methods provided herein are particularly useful for the analysis of biological samples comprising a glycoprotein, such as to generate glycopeptide containing specimens for analysis with a mass spectrometer. The methods provided herein, in some embodiments, enable the analysis of glycopeptides that elute during early or late phases of a reversed-phase chromatographic separation and are typically missed during conventional mass spectrometry approaches. For the situation where a sample contains hydrophilic salts and hydrophilic glycopeptides, it can be challenging to desalt the sample with a C18 solid phase extraction material without removing a significant portion of the hydrophilic glycopeptides that are needed for an analysis of the sample. For example, glycopeptides with an overall hydrophilic character may elute from a reversed-phase material, such as in a desalting column, and are washed away or not introduced to the mass spectrometer during a data acquisition phase of a mass spectrometry technique. In some embodiments, glycopeptides with an overall hydrophobic character may have a high affinity for a reversed-phase material, such as in a desalting column or a chromatography column, and are not properly eluted from a desalting column or during a data acquisition portion of a mass spectrometry technique.

In some embodiments, the method comprises an upstream sample preparation technique, such as for obtaining plasma or serum from a blood sample, performed prior to methods for proteolytically digesting a sample. In some embodiments, the upstream sample preparation technique comprises a cell lysis step. In some embodiments, the upstream sample preparation technique comprises a filtration step. In some embodiments, the upstream sample preparation technique comprises a dilution step. In some embodiments, the upstream sample preparation technique comprises a protein concentration determination step.

In some embodiments, the sample is obtained from an individual. In some embodiments, the sample is obtained from a human individual.

### H5. Systems, Kits, and Compositions

In certain aspects, contemplated herein are systems, kits, and compositions useful for performing the methods described herein. In some embodiments, provided herein is a system, kit, and/or composition useful for performing a proteolytic digestion of a biological sample comprising a glycoprotein as described herein. In some embodiments, provided herein is a system, kit, and/or composition useful for performing a LC-MS analysis of a proteolytic glycopeptide as described herein. In some embodiments, provided herein is a system, kit, and/or composition useful for performing a proteolytic digestion of a biological sample comprising a glycoprotein followed by LC-MS analysis of the proteolytic glycopeptide produced therefrom.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### Section 6—Fibrinogen-Depletion and Use Thereof in Glycoproteomic Analysis

Provided herein, in certain aspects, are methods of processing a blood-derived sample obtained from an individual for a mass spectrometry (MS) technique, such as a glycoproteomic MS technique, the methods comprising admixing the blood-derived sample with one or more defibrination factors to promote formation of a fibrin clot, the one or more defibrination factors comprises one or more members selected from the group consisting of: a clotting co-factor; a clotting enzyme; and a clotting activator and/or an exogenous surface aggregation agent. The disclosure taught herein is based, at least in part, on the inventors' unexpected findings that certain processing techniques using defibrination factors, and combinations thereof, enable the production of fibrinogen-depleted samples having advantageous properties for mass spectrometry biomarker analyses, including for glycopeptides. Similar to known advantages of using serum for biomarker analyses, the fibrinogen-depleted samples provided herein represent a less complex biological sample, such as compared to whole blood, plasma, or serum, and thus enable more consistent study of glycopeptides. As described herein, blood-derived samples (e.g., plasma or serum) can be processed using such defibrination factors to obtain a fibrinogen-depleted sample having mass spectrometry compatibility, including with any of protein digestion, sample clean-up (e.g., desalting), chromatography, and mass spectrometry instrumentation. As demonstrated herein, plasma prepared with various anticoagulants can be defibrinated using the methods taught herein resulting in fibrinogen-depleted samples having a lower comparable complexity and improved uniformity thereby improving confidence in downstream mass spectrometry-based biomarker analyses regardless of the initial sample source. Additionally, the inventors' unexpectedly found that mass spectrometry analyses of the fibrinogen-depleted samples produced herein correlated very well with mass spectrometry analyses of serum samples.

Thus, in some aspects, provided herein is a method of processing a blood-derived sample obtained from an individual for a glycoproteomic mass spectrometry (MS) technique, the method comprising: (a) admixing the blood-derived sample with one or more defibrination factors to promote formation of a fibrin clot, the one or more defibrination factors comprises one or more members selected from the group consisting of: a clotting co-factor; a clotting enzyme; and a clotting activator and/or an exogenous surface aggregation agent; (b) separating the formed fibrin clot from the admixed blood-derived sample to obtain a fibrinogen-depleted sample; and (c) subjecting the fibrinogen-depleted sample to one or more MS preparation techniques to produce a test sample for the glycoproteomic mass spectrometry technique. In some embodiments, the one or more defibrination factors comprises the clotting co-factor. In some embodiments, the clotting co-factor comprises a divalent cation, such as Ca2+ or Mg2+, or a combination thereof. In some embodiments, the one or more defibrination factors comprises the clotting enzyme. In some embodiments, the clotting enzyme is thrombin. In some embodiments, the one or more defibrination factors comprises the clotting activator and/or the exogenous surface aggregation agent. In some embodiments, the clotting activator and/or the exogenous surface aggregation agent is an exogenous surface aggregation agent, such as Kaolin. In some embodiments, the clotting activator and/or the exogenous surface aggregation agent is a clotting activator and exogenous surface aggregation agent, such as silica, e.g., a silica particle.

In other aspects, provided herein is a method of preparing a plasma sample obtained from an individual for a glycoproteomic mass spectrometry technique, the method comprising: (a) admixing the plasma sample with defibrination factors to promote formation of a fibrin clot, the defibrination factors comprising: a clotting co-factor; a clotting enzyme; and a clotting activator and/or an exogenous surface aggregation agent; (b) separating the formed fibrin clot from the admixed plasma sample to obtain a fibrinogen-depleted sample; and (c) subjecting the fibrinogen-depleted sample to one or more MS preparation techniques to produce a test sample for the glycoproteomic mass spectrometry technique. In some embodiments, after following admixing with the blood-derived sample: the clotting co-factor comprises Ca²⁺ at a concentration of about 5 mM to about 25 mM; the clotting enzyme comprises thrombin at a concentration of about 1 unit/mL to 10 units/mL; and the clotting activator and/or the exogenous surface aggregation agent is in an amount of about 50 μg to about 500 μg per 40 μL of the blood-derived sample.

In other aspects, provided herein is a method of analyzing a glycopeptide using a mass spectrometry technique, the method comprising subjecting a fibrinogen-depleted sample, or a derivative thereof, such as obtained using the methods provided herein, to the mass spectrometry technique. In some embodiments, the mass spectrometry technique comprises a multiple-reaction-monitoring (MRM) technique, such as a MRM technique targeting a glycopeptide.

In other aspects, provided herein is a defibrination composition comprising: a clotting co-factor; a clotting enzyme; and a clotting activator and/or an exogenous surface aggregation agent.

In other aspects, provided herein is a vessel (such as a sample tube) comprising a defibrination composition described herein.

### B6. Example Mass Spectrometry and Sample Preparation Workflow

For purposes of orientation and illustration of the description herein, provided in this section are example aspects of sample preparation and mass spectrometry workflows (FIGS. 1A-1C) for analyzing the composition of a peptide and/or glycopeptide using a mass spectrometer. Subsequent sections are provided with more details regarding certain inventive features related to methods for processing a proteolytic digest sample derived from a biological sample comprising a glycoprotein, methods of performing a LC-MS analysis of a proteolytic glycopeptide, and mass spectrometry workflows involving any combination of elements thereof.

FIG. 1A is a schematic of an example workflow 100 for a peptide structure (e.g., including sequence identification) analysis, including of glycopeptides. The workflow 100 may include various operations including, for example, sample collection 102, sample intake 104, sample preparation and mass spectrometry processing 106, and data analysis 108.

Sample collection 102 may include, for example, obtaining a biological sample 112 from an individual 114. A biological sample 112 may take the form of a specimen obtained via one or more sampling methods. A biological sample 112 may be representative of an individual 114 as a whole or of a specific tissue, cell type, or other category or sub-category of interest. In some embodiments, the biological sample 112 includes a whole blood sample 116 obtained via a blood draw or a blood-derived sample, such as obtained from processing of whole blood, e.g., plasma or serum. In some embodiments, the biological sample 112 includes set of aliquoted samples 118 that include, for example, a serum sample, a plasma sample, a blood cell (e.g., white blood cell (WBC), red blood cell (RBC) sample, another type of sample, or a combination thereof. In some embodiments, the biological sample 112 is a plasma sample from the individual 114. In some embodiments, the biological sample 112 is a serum sample from the individual 114. In some embodiments, the biological sample 112 may include nucleotides (e.g., ssDNA, dsDNA, RNA), organelles, amino acids, peptides, proteins, carbohydrates, glycoproteins, or any combination thereof.

Sample intake 104 may include one or more various operations such as, for example, aliquoting, labeling, registering, processing, storing, thawing, and/or other types of operations involved with preparing a sample for sample preparation and mass spectrometry processing. As discussed in more detail herein, in certain aspects, provided is processing methodology (and composition and systems useful therewith) for producing a fibrinogen-depleted sample from a biological sample, such as from plasma or serum. Such fibrinogen-depleted samples can then be used in downstream mass spectrometry based sample preparation and processing techniques 106.

Sample preparation and mass spectrometry processing 106 may include, for example, one or more operations to form set of peptide species 122, such as a proteolytically digested peptide and/or a proteolytically digested glycopeptide. In some embodiments, the sample preparation includes subjecting a biological sample to a proteolytic digestion. Mass spectrometry processing 124 may include, for example, liquid chromatography, introducing species from the sample, and/or derived therefrom, to a mass spectrometer, and data acquisition, such as using a multiple reaction monitoring (MRM) technique. MRM is a mass spectrometry method in which a precursor ion of a particular m/z value, including window thereof, (e.g., peptide analyte) is selected in the first quadrupole (Q1) and transmitted to the second quadrupole (Q2) for fragmentation. The resulting product ions are then transmitted to the third quadrupole (Q3), which detects only product ions with selected predefined m/z values. In some embodiments, the predefined m/z value, including window thereof, selected in the first quadrupole and a predefined m/z value, including window thereof, may be expressed as a MRM transition. Dynamic MRM (dMRM) is a variant of MRM. In dynamic MRM mode, MRM transition lists are scheduled throughout an LC/MS run based on the retention time window for each analyte. In this way, analytes are only monitored while they are eluting from the LC and therefore the MS scan time is not wasted by monitoring the analytes when they are not expected.

Data analysis 108 may include, for example, peptide structure analysis 126, e.g., determining the amino acid sequence of a peptide, determining a site of a post-translational modification, and/or determining a glycan composition and/or structure. In some embodiments, data analysis 108 also includes output generation 110. In some embodiments, output generation 110 may be considered a separate operation from data analysis 108. Output generation 110 may include, for example, generating final output 128 based on the results of peptide structure analysis 126. In some embodiments, the final output 128 may be used for one or more downstream purposes, such as research, diagnosis, and/or treatment, and may be sent to a remote system 130.

In certain aspects, the workflow 100 may optionally exclude one or more of the operations described herein and/or may optionally include one or more other steps or operations other than those described herein (e.g., in addition to and/or instead of those described herein).

FIG. 1B is a schematic of an example workflow 200 for certain sample preparation techniques 106, some of which may be optionally used in methods provided herein, including proteolytic digestion techniques to produce a proteolytic digest sample. In some embodiments, the workflow 200 comprises a denaturation step 202, such as to unfold and/or linearize a polypeptide to expose one or more cleavage sites. In some embodiments, the workflow 200 comprises a reduction step 202, such as to cleave disulfide bonds. In some embodiments, the workflow 200 comprises an alkylation technique 204, such as to modify cysteine residues to prevent reformation of a disulfide bond. In some embodiments, the workflow 200 comprises a proteolytic digestion technique 206, such as to produce proteolytically digested peptides, including proteolytically digested glycopeptides. Box 205 can represent the R group of an amino acid such as, for example, an R group of arginine or lysine that typically will direct a tryptic cleavage. In some embodiments, the workflow 200 may comprise a post-digestion procedure 207, such as any one or more of a desalting technique, addition of a standard, aliquoting, and/or preparation for a mass spectrometry analysis.

FIG. 1C is a schematic of an example workflow for certain mass spectrometry processing techniques 106, some of which may be optionally used in methods provided herein. In some embodiments, the workflow comprises a quantification technique 208 using a mass spectrometer, such as a liquid chromatography-mass spectrometry system. In some embodiments, the workflow comprises a quality control technique 210 configured to optimize data quality. In some embodiments, measures can be put in place allowing only errors within acceptable ranges outside of an expected value. In some embodiments, employing statistical models (e.g., using Westgard rules) can assist in quality control 210. For example, quality control 210 may include, for example, assessing the retention time and abundance of representative peptide species (e.g., glycosylated and/or aglycosylated peptide species) and spiked-in internal standards, in either every sample, or in each quality control sample (e.g., pooled serum digest). In some embodiments, the workflow comprises a peak integration and normalization technique 212 to process the data that has been generated and transform the data into a format for analysis. For example, peak integration and normalization 212 may include converting abundance data for various productions that were detected for a selected peptide species into a single quantification metric (e.g., a relative quantity, an adjusted quantity, a normalized quantity, a relative concentration, an adjusted concentration, or a normalized concentration) for that peptide structure. In some embodiments, peak integration and normalization 212 may be performed using one or more of the techniques described in U.S. Patent Publication No. 2020/0372973A1 and/or US Patent Publication No. 2020/0240996A1, the disclosures of which are hereby incorporated herein by reference in their entireties.

### C6. Fibrinogen Depletion

Provided herein, in certain aspects, are methods of processing a blood-derived sample obtained from an individual to obtain a fibrinogen-depleted sample, wherein the processing comprises admixing the blood-derived sample with one or more defibrination factors. As described herein, the produced fibrinogen-depleted samples are suitable for downstream mass spectrometry sample processing, such as proteolytic digestion, and mass spectrometry analyses, including LC-MS ESI. In some embodiments, reference to a derivative of a fibrinogen-depleted sample reflects that certain additional processing steps have been performed, such as a proteolytic digestion.

In some embodiments, provided is a method of processing a blood-derived sample obtained from an individual for a glycoproteomic mass spectrometry (MS) technique, the method comprising: (a) admixing the blood-derived sample with one or more defibrination factors to promote formation of a fibrin clot, the one or more defibrination factors comprises one or more members selected from the group consisting of: a clotting co-factor; a clotting enzyme; and a clotting activator and/or an exogenous surface aggregation agent; (b) separating the formed fibrin clot from the admixed blood-derived sample to obtain a fibrinogen-depleted sample; and (c) subjecting the fibrinogen-depleted sample to one or more MS preparation techniques to produce a test sample for the glycoproteomic mass spectrometry technique. In some embodiments, the one or more defibrination factors comprise the clotting co-factor and the clotting enzyme. In some embodiments, the one or more defibrination factors comprise the clotting co-factor and the clotting activator and/or the exogenous surface aggregation agent. In some embodiments, the one or more defibrination factors comprise the clotting enzyme and the clotting activator and/or the exogenous surface aggregation agent. In some embodiments, the one or more defibrination factors comprise the clotting co-factor, the clotting enzyme, and the clotting activator and/or the exogenous surface aggregation agent.

In some embodiments, provided is a method of processing a blood-derived sample obtained from an individual for a glycoproteomic mass spectrometry (MS) technique, the method comprising: (a) admixing the blood-derived sample with one or more defibrination factors to promote formation of a fibrin clot, the one or more defibrination factors consist essentially of one or more members selected from the group consisting of: a clotting co-factor; a clotting enzyme; and a clotting activator and/or an exogenous surface aggregation agent; (b) separating the formed fibrin clot from the admixed blood-derived sample to obtain a fibrinogen-depleted sample; and (c) subjecting the fibrinogen-depleted sample to one or more MS preparation techniques to produce a test sample for the glycoproteomic mass spectrometry technique. In some embodiments, the one or more defibrination factors comprise the clotting co-factor and the clotting enzyme. In some embodiments, the one or more defibrination factors comprise the clotting co-factor and the clotting activator and/or the exogenous surface aggregation agent. In some embodiments, the one or more defibrination factors comprise the clotting enzyme and the clotting activator and/or the exogenous surface aggregation agent. In some embodiments, the one or more defibrination factors comprise the clotting co-factor, the clotting enzyme, and the clotting activator and/or the exogenous surface aggregation agent.

In some embodiments, the one or more defibrination factors comprises a clotting co-factor. In some embodiments, the clotting factor comprises a monovalent cation or a divalent cation, or any combination thereof. In some embodiments, the clotting factor comprises a monovalent cation. In some embodiments, the monovalent cation is K⁺ or Na⁺, or any combination thereof. In some embodiments, the clotting co-factor comprises a divalent cation. In some embodiments, the divalent cation is Ca²⁺, Mg²⁺, Zn²⁺, or Cu²⁺, or any combination thereof. In some embodiments, the divalent cation is Ca²⁺. In some embodiments, the clotting co-factor, or source thereof, is calcium chloride, calcium acetate, calcium carbonate, calcium citrate, or calcium gluconate, or any combination thereof.

The clotting co-factors described herein may be added in a range of concentrations, such as to obtain a desired final concentration. In some embodiments, following admixing with a blood-derived sample, the clotting co-factor (such as a divalent cation) has a concentration of about 5 mM to about 25 mM, such as any of about 5 mM to about 20 mM, about 10 mM to about 20 mM, about 5 mM to about 15 mM, about 10 mM to about 20 mM, or about 15 mM to about 25 mM. In some embodiments, following admixing with a blood-derived sample, the clotting co-factor (such as a divalent cation) has a concentration of at least about 5 mM, such as at least about any of 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, or 25 mM. In some embodiments, following admixing with a blood-derived sample, the clotting co-factor (such as a divalent cation) has a concentration of about any of 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, or 25 mM. In some embodiments, following admixing with a blood-derived sample, the clotting co-factor (such as a monovalent cation) has a concentration of about 0.2 M to about 0.5 M, including about any of 0.2 M, 0.25 M, 0.3 M, 0.35 M, 0.4 M, 0.45 M, or 0.5 M. As described herein, in some embodiments the concentration of a clotting co-factor is based on a final desired concentration (such as after the one or more defibrination factors are admixed with a blood-derived sample), and any amounts, concentrations, and forms of the clotting co-factor added are encompassed by the disclosure provided herein, e.g., a stock composition such as a stock solution comprising the clotting co-factor.

In some embodiments, the one or more defibrination factors comprises a clotting enzyme. In some embodiments, clotting enzymes are known in active and zymogen forms. In some embodiments, the clotting enzyme is in an active form. In some embodiments, the clotting enzyme is thrombin. In some embodiments, the clotting enzyme is thrombin in an active form, namely, the thrombin can catalyze the conversion of fibrinogen to fibrin. Thrombin that is useful to the disclosure provided herein may come in many forms. For example, in some embodiments, the clotting enzyme is thrombin isolated from an organism, such as thrombin isolated from an individual, including a human or a non-human animal. In some embodiments, the clotting enzyme is a recombinant thrombin.

The clotting enzymes described herein may be added in a range of concentrations, such as to obtain a desired final concentration or unit based on enzymatic activity (e.g., μmol substrate converted per minute). In some embodiments, following admixing with a blood-derived sample, the clotting enzyme is present in an amount of about 1 unit/mL to about 10 units/mL, such as any of about 3 units/mL to about 7 units/mL, about 1 unit/mL to about 3 units/mL, or about 4 units/mL to about 6 units/mL. In some embodiments, following admixing with a blood-derived sample, the clotting enzyme is present in an amount of at least about 0.5 units/mL, such as at least about 1 unit/mL, 1.5 units/mL, 2 units/mL, 2.5 units/mL, 3 units/mL, 3.5 units/mL, 4 units/mL, 4.5 units/mL, 5 units/mL, 5.5 units/mL, 6 units/mL, 6.5 units/mL, 7 units/mL, 7.5 units/mL, 8 units/mL, 8.5 units/mL, 9 units/mL, 9.5 units/mL, or 10 units/mL. In some embodiments, following admixing with a blood-derived sample, the clotting enzyme is present in an amount of about any of 0.5 units/mL, 1 unit/mL, 1.5 units/mL, 2 units/mL, 2.5 units/mL, 3 units/mL, 3.5 units/mL, 4 units/mL, 4.5 units/mL, 5 units/mL, 5.5 units/mL, 6 units/mL, 6.5 units/mL, 7 units/mL, 7.5 units/mL, 8 units/mL, 8.5 units/mL, 9 units/mL, 9.5 units/mL, or 10 units/mL. As described herein, in some embodiments the concentration of a clotting enzyme is based on a final desired amount (such as after the one or more defibrination factors are admixed with a blood-derived sample), and any amounts, concentrations, and forms of the clotting enzyme added are encompassed by the disclosure provided herein, e.g., a stock composition such as a stock solution comprising the clotting enzyme.

In some embodiments, the one or more defibrination factors comprises a clotting activator and/or an exogenous surface aggregation agent. As described herein, in some embodiments, an agent that is admixed as the one or more defibrination can have one or more functions, and the use of two functional descriptors is not intended to require more than one agent. For example, in some embodiments, the one or more defibrination factors comprises an agent that is a clotting activator and an exogenous surface aggregation agent. In some embodiments, the clotting activator and/or the exogenous surface aggregation agent is an exogenous surface aggregation agent. In some embodiments, the exogenous surface aggregation agent comprises Kaolin. Kaolin, also described as Kaolinite, comprises Al₂Si₂O₅(OH)₄. In some embodiments, Kaolin has an average particle size of about 20 μm to about 40 μm, such as any of about 25 μm to about 35 μm, about 20 μm to about 30 μm, or about 30 μm to about 40 μm. In some embodiments, the clotting activator and/or the exogenous surface aggregation agent is a clotting activator and exogenous surface aggregation agent. In some embodiments, the clotting activator and/or the exogenous surface aggregation agent, such as the clotting activator and exogenous surface aggregation agent, comprises a material having pores with an average size of about 2 nm to about 60 nm, such as any of about 2 nm to about 50 nm, about 2 nm to about 20 nm, about 2 nm to about 10 nm, or about 10 nm to about 50 nm. In some embodiments, the clotting activator and/or the exogenous surface aggregation agent, such as the clotting activator and exogenous surface aggregation agent, comprises a material having pores with an average size of about 60 nm or less, such as about any of 55 nm or less, 50 nm or less, 45 nm or less, 40 nm or less, 35 nm or less, 30 nm or less, 25 nm or less, 20 nm or less, 15 nm or less, 10 nm or less, or 5 nm or less. In some embodiments, the clotting activator and/or the exogenous surface aggregation agent, such as the clotting activator and exogenous surface aggregation agent, comprises silica. In some embodiments, the clotting activator and/or the exogenous surface aggregation agent, such as the clotting activator and exogenous surface aggregation agent, comprises a silica particle. In some embodiments, the silica particle has pores with an average size of about 2 nm to about 60 nm, such as any of about 2 nm to about 50 nm, about 2 nm to about 20 nm, about 2 nm to about 10 nm, about 10 nm to about 50 nm, about 8 nm to about 16 nm, or about 10 nm to about 14 nm. In some embodiments, the silica particle has pores with an average size of about 60 nm or less, such as about any of 55 nm or less, 50 nm or less, 45 nm or less, 40 nm or less, 35 nm or less, 30 nm or less, 25 nm or less, 20 nm or less, 15 nm or less, 10 nm or less, or 5 nm or less. In some embodiments, the silica particle has an diameter of about 150 μm or less, such as about any of 140 μm or less, 130 μm or less, 120 μm or less, 110 μm or less, 100 μm or less, 90 μm or less, 80 μm or less, 70 μm or less, 60 μm or less, 50 μm or less, 40 μm or less, 30 μm or less, 20 μm or less, 15 μm or less, or 10 μm or less. In some embodiments, the silica particle has a surface area of about 200 m²/g to about 500 m²/g. In certain embodiments, particle size of components is as measured by dynamic light scattering (DLS).

The clotting activators and/or the exogenous surface aggregation agents described herein may be added in a range of concentrations, such as to obtain a desired final concentration or characteristic, such as surface area. In some embodiments, the clotting activator and/or the exogenous surface aggregation agent is admixed with the blood-derived sample at an amount of about 50 μg to about 500 μg, such as any of 75 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, or 475 μg, per 40 μL of the blood-derived sample.

In some embodiments, the one or more defibrination factors comprise the clotting co-factor (such as Ca²⁺) and the clotting enzyme (such as thrombin), wherein, following admixing with the blood-derived sample, the clotting co-factor has a concentration of about 5 mM to about 25 mM, and wherein, following admixing with the blood-derived sample, the clotting enzyme has a concentration of about 1 unit/mL to 10 units/mL. In some embodiments, the one or more defibrination factors comprise the clotting co-factor (such as Ca²⁺) and the clotting activator and/or the exogenous surface aggregation agent (such as Kaolin or a silica particle), wherein, following admixing with the blood-derived sample, the clotting co-factor has a concentration of about 5 mM to about 25 mM, and wherein the clotting activator and/or the exogenous surface aggregation agent is admixed with the blood-derived sample at an amount of about 50 μg to about 500 μg per 40 μL of the blood-derived sample. In some embodiments, the one or more defibrination factors comprise the clotting enzyme (such as thrombin) and the clotting activator and/or the exogenous surface aggregation agent (such as Kaolin or a silica particle), wherein, following admixing with the blood-derived sample, the clotting enzyme has a concentration of about 1 unit/mL to 10 units/mL, and wherein the clotting activator and/or the exogenous surface aggregation agent is admixed with the blood-derived sample at an amount of about 50 μg to about 500 μg per 40 μL of the blood-derived sample. In some embodiments, the one or more defibrination factors comprise the clotting co-factor (such as Ca²⁺), the clotting enzyme (such as thrombin), and the clotting activator and/or the exogenous surface aggregation agent (such as Kaolin or a silica particle), wherein, following admixing with the blood-derived sample, the clotting co-factor has a concentration of about 5 mM to about 25 mM, wherein, following admixing with the blood-derived sample, the clotting enzyme has a concentration of about 1 unit/mL to 10 units/mL, and wherein the clotting activator and/or the exogenous surface aggregation agent is admixed with the blood-derived sample at an amount of about 50 μg to about 500 μg per 40 μL of the blood-derived sample.

The present disclosure encompasses the various manners in which the one or more defibrination factors is admixed with the blood-derived sample. For example, in some embodiments, when more than one defibrination factor is admixed with the blood-derived sample sequentially. In some embodiments, when more than one defibrination factor is admixed with the blood-derived sample simultaneously. In some embodiments, at least one of the one or more defibrination factors is added to a vessel containing the blood-derived sample. In some embodiments, the blood-derived sample is added to a vessel containing at least one of the one or more defibrination factors.

The methods provided herein include, in certain embodiments, incubation periods following contact of one or more defibrination factors with a blood-derived sample. Incubation periods may be assessed by determining/monitoring the rate and/or degree of fibrin formation (including by measuring the amount of fibrinogen not converted to fibrin using the methods described herein). In some embodiments, the incubation period is about 1 minute to about 30 minutes. In some embodiments, the incubation period is at least about 30 seconds, including about any of 30 minutes, 1 hour, 2 hours, 3 hours, 6 hours, 8 hours, 10 hours, 12 hours, or 18 hours.

The method provided herein, in certain aspects, include a step of separating a formed fibrin clot to obtain a fibrinogen-depleted sample. In some embodiments, the separating the formed fibrin clot to obtain the fibrinogen-depleted sample comprises subjecting the admixed blood-derived sample with the one or more defibrination factors to a centrifugation technique and/or a filtration technique. In some embodiments, the separating comprises a centrifugation technique. The centrifugation techniques encompassed herein are suitable for pelleting a fibrin clot formed in the taught methods while substantially maintaining desired polypeptide content in a formed supernatant, such as a desired glycopeptide biomarker. In some embodiments, the centrifugation technique comprises centrifugation at from 1,500 G to 2,500 G. In some embodiments, the centrifugation technique comprises centrifugation at 2,000 G. In some embodiments, the centrifugation technique comprises centrifugation from 15 minutes to 45 minutes. In some embodiments, the centrifugation technique comprises centrifugation for 30 minutes. In some embodiments, the centrifugation technique comprises centrifugation at 2,000 G for 30 minutes. In some embodiments, the separating comprises a filtration technique. In some embodiments, the separating comprises subjecting the admixed blood-derived sample with the one or more defibrination factors to a supernatant collection technique (such as following a centrifugation technique). In some embodiments, the separating comprises a manual step, such as pipetting a supernatant. In some embodiments, the separating comprises an automated step, such as involving automation of the steps described herein, e.g., centrifugation, filtration, and supernatant removal.

In some embodiments, the method provided herein enable the production of a fibrinogen-depleted sample, wherein the fibrinogen-depleted sample is depleted of at least about 80%, such as at least about any of 81%, 82%, 83%, 84%, 85%, 86%, 87%, 88%, 89%, 90%, 91%, 92%, 93%, 94%, 95%, 96%, 97%, 98%, or 99%, of the fibrinogen as compared to a blood-derived sample (such as the starting material to form the fibrinogen-depleted sample) or a reference standard. In some embodiments, the fibrinogen-depleted sample contains 0.005 mg/mL of fibrinogen or less, including about any of 0.001 mg/mL of fibrinogen or less or 0.0005 mg/mL of fibrinogen of less.

In certain aspects of the methods described herein, a blood-derived sample is processed. As used herein, a blood-derived sample is intended to exclude unprocessed whole blood obtained from an individual, such as a human. In some embodiments, unprocessed whole blood is collected into a tube containing anti-coagulants, and then processed into a blood-derived sample such as plasma. In some embodiments, the blood-derived sample is a plasma sample. In some embodiments, the plasma sample has been treated with, or contains, an anticoagulant. In some embodiments, the plasma sample has been treated with, or contains, any one or more of the following: a citrate, an ACD (anticoagulant citrate dextrose; including any type thereof, such as ACD-A), Streck, EDTA (ethylenediaminetetraacetic acid), Heparin or Li-Heparin, oxalate fluoride, or a citrate phosphate dextrose adenine (CPDA). In some embodiments, the blood-derived sample is a serum sample.

In certain aspects, provided is a method of preparing a plasma sample obtained from an individual for a glycoproteomic mass spectrometry technique, the method comprising: (a) admixing the plasma sample with defibrination factors to promote formation of a fibrin clot, the defibrination factors comprising: a clotting co-factor; a clotting enzyme; and a clotting activator and/or an exogenous surface aggregation agent; (b) separating the formed fibrin clot from the admixed plasma sample to obtain a fibrinogen-depleted sample; and (c) subjecting the fibrinogen-depleted sample to one or more MS preparation techniques to produce a test sample for the glycoproteomic mass spectrometry technique. In some embodiments, after following admixing with the blood-derived sample: the clotting co-factor comprises Ca²⁺ at a concentration of about 5 mM to about 25 mM (such as 10 mM, 15, mM, or 20 mM); the clotting enzyme comprises thrombin at a concentration of about 1 unit/mL to 10 units/mL (such as 5 units/mL); and the clotting activator and/or the exogenous surface aggregation agent is in an amount of about 50 μg to about 500 μg (such as 100 μg or 200 μg) per 40 μL of the blood-derived sample.

In certain aspects, provided herein are defibrination compositions comprising one or more of, including all three of: a clotting co-factor; a clotting enzyme; and a clotting activator and/or an exogenous surface aggregation agent. Encompassed in the description herein are various defibrination composition configured to provide one or more defibrination factors at set amounts once admixed with a blood-derived sample. For example, in some embodiments, the defibrination composition comprises components in amounts such that admixing with a set amount of a blood-derived sample (such as 40 μL of plasma or serum), will provide the clotting co-factor comprises Ca²⁺ at a concentration of about 5 mM to about 25 mM (such as 10 mM, 15, mM, or 20 mM); the clotting enzyme comprises thrombin at a concentration of about 1 unit/mL to 10 units/mL (such as 5 units/mL); and the clotting activator and/or the exogenous surface aggregation agent is in an amount of about 50 μg to about 500 μg (such as 100 μg or 200 μg) per 40 μL of the blood-derived sample. Also encompassed are various forms of defibrination compositions, including a dry power, a lyophilized substance, or a solution.

In certain aspects, provided herein is a vessel (such as a sample tube) comprising a defibrination composition provided herein. A diverse array of vessels are encompassed by the description provided herein. In some embodiments, the vessel is a blood vial. In some embodiments, the vessel is a plasma or serum vial. In some embodiments, the vessel is an Eppendorf tube. In some embodiments, the vessel is a multi-well plate, such as a 96-well plate.

### D6. Methods for Proteolytically Digesting a Fibrinogen-Depleted Sample, or a Derivative Thereof

In certain aspects, provided herein are methods of proteolytically digesting a fibrinogen-depleted sample, or a derivative thereof, including a fibrinogen-depleted sample comprising a glycoprotein, the methods comprising subjecting the fibrinogen-depleted sample, or the derivative thereof, to a thermal denaturation technique. As described herein, derivative(s) of a fibrinogen-depleted sample is intended to reflect that downstream process(es) may result in modification of the original fibrinogen-depleted sample, and as such additional descriptors are used herein to communicate various actions taken thereon. Proteases are enzymes that cleave polypeptides at, generally, specific cleavage motifs. For example, trypsin is a serine protease that generally cleaves polypeptides at the carboxyl side (C-terminal side) of lysine and arginine residues. A glycan of a glycopeptide may present a steric hindrance to a protease, thereby inhibiting complete protease digestion of a fibrinogen-depleted sample, or a derivative thereof, comprising a glycoprotein. Without being bound to this theory, it is believed that the methods taught herein improve polypeptide unfolding, such as linearization, and provide protease access to cleavage sites thereby providing methods for more complete proteolytic digestion of glycoproteins.

In some aspects, provided is a method comprising subjecting a fibrinogen-depleted sample, or a derivative thereof, to a thermal denaturation technique to produce a denatured sample.

In other aspects, provided is a method comprising subjecting a fibrinogen-depleted sample, or a derivative thereof, to a thermal denaturation technique to produce a denatured sample followed by a proteolytic digestion technique to produce a proteolytic digest sample comprising a proteolytic glycopeptide. In some embodiments, the method comprises quenching one or more proteases used in a proteolytic digestion technique prior to a downstream technique, such as LC-MS.

In other aspects, provided herein is a method comprising: subjecting a fibrinogen-depleted sample, or a derivative thereof, to a thermal denaturation technique to produce a denatured sample; subjecting the denatured sample to a reduction technique to produce a reduced sample; subjecting the reduced sample to an alkylation technique to produce an alkylated sample; and subjecting the alkylated sample to a proteolytic digestion technique to produce a proteolytic digest sample comprising the proteolytic glycopeptide. In some embodiments, the method comprises quenching an alkylating agent used in the alkylation technique prior to subjecting an alkylated sample to a proteolytic digestion technique. In some embodiments, the method comprises quenching one or more proteases used in a proteolytic digestion technique prior to a downstream technique, such as LC-MS.

In some embodiments, the proteolytic digestion methods described herein produce a proteolytic digestion sample, including one comprising a proteolytic glycopeptide, having a digestion completion rate of at least about 70%, such as at least about any of 75%, 80%, 85%, 90%, 91%, 92%, 93%, 94%, 95%, 96%, 97%, 98%, or 99%. In some embodiments, the proteolytic digestion methods described herein produce a proteolytic digestion sample, including one comprising a proteolytic glycopeptide, wherein the sample volume loss is 10% or less, such as 9% or less, 8% or less, 7% or less, 6% or less, 5% or less, 4% or less, 3% or less, 2% or less, or 1% or less, based on all volumes added in producing the proteolytic digestion sample.

In the following sections, additional description of the various aspects of the proteolytic digestion techniques is provided. Such description in a modular fashion is not intended to limit the scope of the disclosure, and based on the teachings provided herein one of ordinary skill in the art will readily appreciate that certain modules can be integrated, at least in part. The section heading used herein are for organizational purposes only and are not to be construed as limiting the subject matter described.

### I. Thermal Denaturation Techniques

In certain aspects, the methods provided herein comprise performing a thermal denaturation technique. Thermal denaturation techniques, generally speaking, change certain polypeptides conformational structures, such as by unfolding and/or linearizing a polypeptide, to enable protease access to cleavage sites. Thermal denaturation techniques described herein comprise subjecting a sample, or a derivative thereof (e.g., a sample diluted with a buffer), to a thermal treatment of about 60° C. to about 100° C. for thermal denaturation incubation time of at least about 1 minute. In some embodiments, the thermal denaturation technique is not performed concurrently with a chemical denaturation technique, such as using high concentrations of denaturing agent, e.g., 6M urea. In some embodiments, the method does not include use of a chemical denaturation technique.

In some embodiments, the thermal denaturation incubation time is performed at a temperature of about 60° C. to about 100° C., such as any of about 70° C. to about 100° C., about 80° C. to about 100° C., about 90° C. to about 100° C., about 95° C. to about 100° C., or about 85° C. to about 95° C. In some embodiments, the thermal denaturation incubation time is performed at a temperature of at least about 60° C., such as at least about any of 65° C., 70° C., 75° C., 80° C., 85° C., 90° C., 95° C., or 100° C. In some embodiments, the thermal denaturation incubation time is performed at a temperature of about 100° C. or less, such as about any of 95° C. or less, 90° C. or less, 85° C. or less, 80° C. or less, 75° C. or less, 70° C. or less, 65° C. or less, 60° C. or less. In some embodiments, the thermal denaturation incubation time is performed at a temperature of about any of 60° C., 65° C., 70° C., 75° C., 80° C., 85° C., 90° C., 91° C., 92° C., 93° C., 94° C., 95° C., 96° C., 97° C., 98° C., 99° C., or 100° C.

In some embodiments, the thermal denaturation incubation time is about 1 minute to about 15 minutes, such as any of about 1 minute to about 5 minutes, about 1 minute to about 10 minutes, about 2.5 minutes to about 7.5 minutes, or about 5 minutes to about 15 minutes. In some embodiments, the thermal denaturation incubation time is at least about 1 minute, such as at least about any of 1.5 minutes, 2 minutes, 2.5 minutes, 3 minutes, 3.5 minutes, 4 minutes, 4.5 minutes, 5 minutes, 5.5 minutes, 6 minutes, 6.5 minutes, 7 minutes, 7.5 minutes, 8 minutes, 8.5 minutes, 9 minutes, 9.5 minutes, 10 minutes, 11 minutes, 12 minutes, 13 minutes, 14 minutes, or 15 minutes. In some embodiments, the thermal denaturation incubation time is about 15 minutes or less, such as about any of 14 minutes or less, 13 minutes or less, 12 minutes or less, 11 minutes or less, 10 minutes or less, 9.5 minutes or less, 9 minutes or less, 8.5 minutes or less, 8 minutes or less, 7.5 minutes or less, 7 minutes or less, 6.5 minutes or less, 6 minutes or less, 5.5 minutes or less, 5 minutes or less, 4.5 minutes or less, 4 minutes or less, 3.5 minutes or less, 3 minutes or less, 2.5 minutes or less, 2 minutes or less, 1.5 minutes or less, or 1 minute or less. In some embodiments, the thermal denaturation incubation time is about any of 1 minute, 1.5 minutes, 2 minutes, 2.5 minutes, 3 minutes, 3.5 minutes, 4 minutes, 4.5 minutes, 5 minutes, 5.5 minutes, 6 minutes, 6.5 minutes, 7 minutes, 7.5 minutes, 8 minutes, 8.5 minutes, 9 minutes, 9.5 minutes, 10 minutes, 11 minutes, 12 minutes, 13 minutes, 14 minutes, or 15 minutes.

In some embodiments, the thermal denaturation technique comprises a thermal denaturation incubation time of about 1 minute to about 15 minutes, such as about any of 1 minute, 2 minutes, 3 minutes, 4 minutes, 5 minutes, 6 minutes, 7 minutes, 8 minutes, 9 minutes 10 minutes, 11 minutes, 12 minutes, 13 minutes, 14 minutes, or 15 minutes, wherein the thermal denaturation incubation is performed at a temperature of about 60° C. to about 100° C., such about any of 60° C., 65° C., 70° C., 75° C., 80° C., 85° C., 90° C., 91° C., 92° C., 93° C., 94° C., 95° C., 96° C., 97° C., 98° C., 99° C., or 100° C.

The thermal denaturation incubation temperature can be controlled by numerous techniques and combinations thereof. In some embodiments, the thermal denaturation incubation temperature is controlled by a water bath. In some embodiments, the thermal denaturation incubation temperature is controlled by a heat block. In some embodiments, the thermal denaturation incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the thermal denaturation incubation time temperature are controlled by a technique described herein, such as cooling at room temperature or via the thermocycler.

In some embodiments, the thermal denaturation technique comprises subjecting a sample, or a derivative thereof, e.g., a sample diluted in a buffer, to a thermal cycle. In some embodiments, the thermal cycle comprises subjecting the sample, or a derivative thereof, to one or more of: (a) a block starting temperature (b) block set temperature (the temperature for the thermal denaturation incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 60° C. to about 100° C., including about any of 60° C., 65° C., 70° C., 75° C., 80° C., 85° C., 90° C., 91° C., 92° C., 93° C., 94° C., 95° C., 96° C., 97° C., 98° C., 99° C., or 100° C. In some embodiments, the thermal cycle comprises: (a) a set block temperature of about 60° C. to about 100° C., including about any of 60° C., 65° C., 70° C., 75° C., 80° C., 85° C., 90° C., 91° C., 92° C., 93° C., 94° C., 95° C., 96° C., 97° C., 98° C., 99° C., or 100° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 50° C., such as any of 15° C. to about 25° C., about 20° C. to about 30° C., or about 20° C. to about 25° C., including about any of 20° C., 21° C., 22° C., 23° C., 24° C., 25° C., (b) a set block temperature of about 60° C. to about 100° C., such about any of 60° C., 65° C., 70° C., 75° C., 80° C., 85° C., 90° C., 91° C., 92° C., 93° C., 94° C., 95° C., 96° C., 97° C., 98° C., 99° C., or 100° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C. In some embodiments, the lid temperature during the thermal cycle may be the same respective temperature of the block during the thermal cycle or a temperature greater than the temperature of the block during the thermal cycle. In some embodiments, the lid temperature during the thermal cycle may be the same respective temperature of the block during the thermal cycle or a temperature greater than the temperature of the block during the thermal cycle.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

In some embodiments, the method further comprises admixing an amount of a fibrinogen-depleted sample, or a derivative thereof, and a buffer prior to the thermal denaturation technique (e.g., the buffered sample is subjected to a thermal denaturation technique described herein). In some embodiment, the amount (as assessed based on the final concentration in the sample containing solution containing solution) of the buffer is about 1 mM to about 100 mM, such as any of about 20 mM to about 80 mM, about 30 mM to about 70 mM, or about 40 mM to about 60 mM. In some embodiment, the amount of the buffer is about any of 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some embodiments, the buffer is selected from the group consisting of ammonium bicarbonate, ammonium acetate, ammonium formate, triethylammonium bicarbonate, and Tris-HCl, or any combination thereof.

In some embodiments, the method further comprises determining the protein concentration in a fibrinogen-depleted sample, or a derivative thereof.

### II. Reduction Techniques

In certain aspects, the methods provided herein comprise performing a reduction technique. In some embodiments, the reduction technique is performed on a sample, or a derivative thereof, following thermal denaturation. Reduction techniques, generally speaking, reduce (e.g., cleave) disulfide linkages between cysteine residues of one or more polypeptides to reduce the presence of polypeptide conformations that inhibit or prevent protease cleavage of the one or more polypeptides. Reduction techniques described herein comprise subjecting a sample, or a derivative thereof (e.g., a denatured sample), to an amount of a reducing agent and incubating for a reducing incubation time performed at a temperature or range thereof.

In some embodiments, the reducing agent is dithiothreitol (DTT), tris(2-carboxyethyl) phosphine (TCEP), beta-mercaptoethanol (BME), or a cysteine, or any mixture thereof.

In some embodiments, the amount (as assessed based on the final concentration in the sample containing solution containing solution) of a reducing agent, e.g., DTT, used in a reduction technique is about 1 mM to about 100 mM, such as any of about 1 mM to about 40 mM, about 1 mM to about 30 mM, about 5 mM to about 25 mM, about 10 mM, to about 20 mM, 20 mM to about 80 mM, about 30 mM to about 70 mM, or about 40 mM to about 60 mM. In some embodiments, the amount of reducing agent used in a reduction technique is at least about 1 mM, such as at least about any of 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some embodiments, the amount of reducing agent used in a reduction technique is about 100 mM or less, such as about any of 95 mM or less, 90 mM or less, 85 mM or less, 80 mM or less, 75 mM or less, 70 mM or less, 65 mM or less, 60 mM or less, 55 mM or less, 50 mM or less, 45 mM or less, 40 mM or less, 35 mM or less, 30 mM or less, 25 mM or less, 20 mM or less, 19 or less, 18 or less, 17 or less, 16 or less, 15 or less, 14 or less, 13 or less, 12 or less, 11 or less, 10 or less, 9 or less, 8 or less, 7 or less, 6 or less, 5 or less, 4 or less, 3 or less, 2 or less, or 1 or less. In some embodiments, the amount of reducing agent used in a reduction technique is about any of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In some embodiments, the reduction incubation time is about 10 minutes to about 120 minutes, such as any of about 30 minutes to about 60 minutes, about 40 minutes to about 60 minutes, about 45 minutes to about 55 minutes. In some embodiments, the reduction incubation time is at least about 20 minutes, such as at least about any of 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes. In some embodiments, the reduction incubation time is about 120 minutes or less, such as about any of 115 minutes or less, 110 minutes or less, 105 minutes or less, 100 minutes or less, 95 minutes or less, 90 minutes or less, 85 minutes or less, 80 minutes or less, 75 minutes or less, 70 minutes or less, 65 minutes or less, 60 minutes or less, 55 minutes or less, 50 minutes or less, 45 minutes or less, 40 minutes or less, 35 minutes or less, 30 minutes or less, or 25 minutes or less. In some embodiments, the reduction incubation time is about any of 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes.

In some embodiments, the reduction incubation time is performed at a temperature of about 20° C. to about 100° C., such as any of about 40° C. to about 80° C., about 50° C. to about 70° C., about 50° C. to about 60° C., about 55° C. to about 65° C., or about 60° C. to about 70° C. In some embodiments, the reduction incubation time is performed at a temperature of at least about 20° C., such as at least about any of 25° C., 30° C., 35° C., 40° C., 45° C., 50° C., 55° C., 60° C., 65° C., 70° C., 75° C., 80° C., 85° C., 90° C., or 95° C. In some embodiments, the reduction incubation time is performed at a temperature of about 95° C. or less, such as about any of 90° C. or less, 85° C. or less, 80° C. or less, 75° C. or less, 70° C. or less, 65° C. or less, 60° C. or less, 55° C. or less, 50° C. or less, 45° C. or less, 40° C. or less, 35° C. or less, 30° C. or less, or 25° C. or less. In some embodiments, the reduction incubation time is performed at a temperature of about any of 20° C., 25° C., 30° C., 35° C., 40° C., 45° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 75° C., 80° C., 85° C., 90° C., or 95° C. In some embodiments, the reduction incubation time is performed at a room temperature.

In some embodiments, the reduction technique comprises a reduction incubation time of about 30 minutes to about 70 minutes, such as about any of 35 minutes, 40 minutes, 45 minutes, 50 minutes, 55 minutes, 60 minutes, or 65 minutes, wherein the reduction incubation time is performed at a temperature of about 50° C. to about 70° C., such about any of 55° C., 60° C., or 65° C. In some embodiments, the reduction technique comprises use of an amount (as assessed based on the final concentration in the sample containing solution) of a reducing agent, e.g., DTT, of about 5 mM to about 25 mM, such as any of about 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, or 24 mM, and a reduction incubation time of about 30 minutes to about 70 minutes, such as about any of 35 minutes, 40 minutes, 45 minutes, 50 minutes, 55 minutes, 60 minutes, or 65 minutes, wherein the reduction incubation time is performed at a temperature of about 50° C. to about 70° C., such about any of 55° C., 60° C., or 65° C.

The reduction incubation temperature can be controlled by numerous techniques and combinations thereof. In some embodiments, the reduction incubation temperature is controlled by an ambient temperature, such as room temperature. In some embodiments, the reduction incubation temperature is controlled by a water bath. In some embodiments, the reduction incubation temperature is controlled by a heat block. In some embodiments, the reduction incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the reduction incubation time temperature are controlled by a technique described herein, such as cooling at room temperature or a ramp rate.

In some embodiments, the reduction technique comprises subjecting a sample, or a derivative thereof, e.g., a denatured sample, to a thermal cycle. In some embodiments, the thermal cycle comprises subjecting the sample, or a derivative thereof, to one or more of: (a) a block starting temperature (b) block set temperature (the temperature for the reduction incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 20° C. to about 100° C., such as any of 40° C. to about 80° C., about 50° C. to about 70° C., about 50° C. to about 60° C., about 55° C. to about 65° C., or about 60° C. to about 70° C., including about any of 50° C., 55° C., 60° C., 65° C., or 70° C. In some embodiments, the thermal cycle comprises: (a) a set block temperature of about 20° C. to about 100° C., such as any of about 40° C. to about 80° C., about 50° C. to about 70° C., about 50° C. to about 60° C., about 55° C. to about 65° C., or about 60° C. to about 70° C., including about any of 50° C., 55° C., 60° C., 65° C., or 70° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 60° C., such as any of about 15° C. to about 50° C., about 20° C. to about 40° C., about 20° C. to about 30° C., or about 20° C. to about 25° C., (b) a set block temperature of about 20° C. to about 100° C., such as any of about 40° C. to about 80° C., about 50° C. to about 70° C., about 50° C. to about 60° C., about 55° C. to about 65° C., or about 60° C. to about 70° C., including about any of 50° C., 55° C., 60° C., 65° C., or 70° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

In some embodiments, the reduction technique described herein is completed simultaneously with a thermal denaturation step. For example, the combined thermal denaturation technique and reduction technique comprises adding a reducing agent to a sample, or a derivative thereof, and then subjecting the sample, or the derivative thereof, to a temperature of about 60° C. to about 100° C., such about any of 60° C., 65° C., 70° C., 75° C., 80° C., 85° C., 90° C., 91° C., 92° C., 93° C., 94° C., 95° C., 96° C., 97° C., 98° C., 99° C., or 100° C., for an incubation time of at least about 1 minute, such as at least about any of 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes, or 60 minutes. In some embodiments, the combined thermal denaturation technique and reduction technique comprises adding a reducing agent to a sample, or a derivative thereof, and then subjecting the sample, or the derivative thereof, to a temperature of about 90° C. to about 100° C., for an incubation time of about 40 minutes to about 60 minutes, including 50 minutes.

### III. Alkylation Techniques

In certain aspects, the methods provided herein comprise performing an alkylation technique. In some embodiments, the alkylation technique is performed on a sample, or a derivative thereof, following the performance of a reduction technique. Alkylation techniques, generally speaking, prevent the reformation of one or more disulfide linkages between, e.g., cysteine residues of one or more polypeptides. This is done by, e.g., the addition of an acetamide moiety to the sulfur of a cysteine residue thereby producing an alkylated polypeptide. Alkylation techniques may reduce the presence of polypeptide conformations that inhibit or prevent protease cleavage of the one or more polypeptides. Alkylation techniques described herein comprise subjecting a sample, or a derivative thereof (e.g., a reduced sample), to an amount of an alkylating agent and incubating for an alkylation incubation time performed at a temperature or range thereof. In some embodiments, the method comprises subjecting a denatured sample to a reduction technique followed by an alkylation technique prior to performing a proteolytic digestion technique.

In some embodiments, the alkylating agent is iodoacetamide (IAA), 2-chloroacetamide, an acetamide salt, or any mixture thereof.

In some embodiments, the amount (as assessed based on the final concentration in the sample containing solution) of an alkylating agent, e.g., IAA, used in an alkylation technique is about 10 mM to about 100 mM, such as any of about 10 mM to about 50 mM, about 20 mM to about 40 mM, about 20 mM to about 36 mM, about 15 mM to about 25 mM, about 20 mM to about 25 mM, about 20 mM to about 80 mM, about 30 mM to about 70 mM, or about 40 mM to about 60 mM. In some embodiments, the amount of an alkylating agent used in an alkylation technique is at least about 10 mM, such as at least about any of 15 mM, 16 mM, 17 mM, 18 mM, 19 mM, 20 mM, 21 mM, 22 mM, 23 mM, 24 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In some embodiments, the amount of an alkylating agent used in an alkylation technique is about 100 mM or less, such as about any of 95 mM or less, 90 mM or less, 85 mM or less, 80 mM or less, 75 mM or less, 70 mM or less, 65 mM or less, 60 mM or less, 55 mM or less, 50 mM or less, 45 mM or less, 40 mM or less, 35 mM or less, 30 mM or less, 25 mM or less, 24 mM or less, 23 mM or less, 22 mM or less, 21 mM or less, 20 mM or less, 19 mM or less, 18 mM or less, 17 mM or less, 16 mM or less, 15 mM or less, or 10 mM or less. In some embodiments, the amount of an alkylating agent used in an alkylation technique is about any of 10 mM, 15 mM, 20 mM, 20.5 mM, 21 mM, 21.5 mM, 22 mM, 22.5 mM, 23 mM, 23.5 mM, 24 mM, 24.5 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In some embodiments, the alkylation incubation time is about 5 minutes to about 60 minutes, such as any of about 10 minutes to about 50 minutes, about 20 minutes to about 40 minutes, about 25 minutes to about 35 minutes. In some embodiments, the alkylation incubation time is at least about 5 minutes, such as at least about any of 10 minutes, 15 minutes, 20 minutes, 25 minutes, 30 minutes, 35 minutes, 40 minutes, 45 minutes, 50 minutes, 55 minutes, or 60 minutes. In some embodiments, the alkylation incubation time is about 60 minutes or less, such as about any of 55 minutes or less, 50 minutes or less, 45 minutes or less, 40 minutes or less, 35 minutes or less, 30 minutes or less, 25 minutes or less, 20 minutes or less, 15 minutes or less, 10 minutes or less, or 5 minutes or less. In some embodiments, the alkylation incubation time is about any of 5 minutes, 10 minutes, 15 minutes, 20 minutes, 25 minutes, 30 minutes, 35 minutes, 40 minutes, 45 minutes, 50 minutes, 55 minutes, or 60 minutes.

In some embodiments, the alkylation incubation time is performed at a temperature of about 15° C. to about 100° C., such as any of about 15° C. to about 80° C., about 15° C. to about 60° C., about 15° C. to about 35° C., about 20° C. to about 30° C., or about 20° C. to about 25° C. In some embodiments, the alkylation incubation time is performed at a temperature of at least about 15° C., such as at least about any of 20° C., 21° C., 22° C., 23° C., 24° C., 25° C., 30° C., 35° C., 40° C., 45° C., 50° C., 55° C., 60° C., 65° C., 70° C., 75° C., 80° C., 85° C., 90° C., or 95° C. In some embodiments, the alkylation incubation time is performed at a temperature of about 95° C. or less, such as about any of 90° C. or less, 85° C. or less, 80° C. or less, 75° C. or less, 70° C. or less, 65° C. or less, 60° C. or less, 55° C. or less, 50° C. or less, 45° C. or less, 40° C. or less, 35° C. or less, 30° C. or less, 25° C. or less, 24° C. or less, 23° C. or less, 22° C. or less, 21° C. or less, or 20° C. or less. In some embodiments, the alkylation incubation time is performed at a temperature of about any of 15° C., 16° C., 17° C., 18° C., 19° C., 20° C., 21° C., 22° C., 23° C., 24° C., 25° C., 30° C., 35° C., 40° C., 45° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 75° C., 80° C., 85° C., 90° C., or 95° C. In some embodiments, the alkylation incubation time is performed at a room temperature.

In some embodiments, the alkylation technique comprises an alkylation incubation time of about 5 minutes to about 60 minutes, such as about any of 15 minutes, 20 minutes, 25 minutes, 30 minutes, 35 minutes, or 40 minutes, wherein the alkylation incubation time is performed at a temperature of about 15° C. to about 30° C., such about any of 20° C., 21° C., 22° C., 23° C., 24° C., 25° C. In some embodiments, the alkylation technique comprises use of an amount (containing solution based on the final concentration in the sample) of an alkylating agent, e.g., IAA, of about 15 mM to about 40 mM, such as any of about 20 mM, 20.5 mM, 21 mM, 21.5 mM, 22 mM, 22.5 mM, 23 mM, 23.5 mM, 24 mM, 24.5 mM, 25 mM, 25.5 mM, 26 mM, 26.5 mM, 27 mM, 27.5 mM, 28 mM, 28.5 mM, 29 mM, 29.5 mM, 30 mM, 30.5 mM, 31 mM, 31.5 mM, 32 mM, 32.5 mM, 33 mM, 33.5 mM, 34 mM, 34.5 mM, 35 mM, 35.5 mM, 36 mM, 36.5 mM, 37 mM, 37.5 mM, 38 mM, and an alkylation incubation time of about 5 minutes to about 60 minutes, such as about any of 15 minutes, 20 minutes, 25 minutes, 30 minutes, 35 minutes, or 40 minutes, wherein the alkylation incubation time is performed at a temperature of about 15° C. to about 30° C., such about any of 20° C., 21° C., 22° C., 23° C., 24° C., or 25° C.

The alkylation incubation temperature can be controlled by numerous techniques and combinations thereof. In some embodiments, the alkylation incubation temperature is controlled by an ambient temperature, such as room temperature. In some embodiments, the alkylation incubation temperature is controlled by a water bath. In some embodiments, the alkylation incubation temperature is controlled by a heat block. In some embodiments, the alkylation incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the alkylation incubation time temperature are controlled by a technique described herein, such as cooling at room temperature or a ramp rate.

In some embodiments, the alkylation technique comprises subjecting a sample, or a derivative thereof, e.g., a denatured sample, to a thermal cycle. In some embodiments, the thermal cycle comprises subjecting the sample, or a derivative thereof, to one or more of: (a) a block starting temperature (b) block set temperature (the temperature for the alkylation incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., or 25° C. In some embodiments, the thermal cycle comprises: (a) a set block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., 25° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., 25° C., (b) a set block temperature of about 15° C. to about 30° C., such as any of 15° C. to about 25° C., about 20° C. to about 30° C., or about 20° C. to about 25° C., including about any of 20° C., 21° C., 22° C., 23° C., 24° C., 25° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C.

In some embodiments, the alkylation technique further comprises quenching the alkylating agent comprising use of a neutralizing agent. In some embodiments, the neutralizing agent is a reducing agent. In some embodiments, the reducing agent is dithiothreitol (DTT), tris(2-carboxyethyl) phosphine (TCEP), beta-mercaptoethanol (BME), or a cysteine, or any mixture thereof. In some embodiments, the neutralizing agent is added in an amount to fully quench the amount of the alkylating agent, such as in an amount greater than or equal to a molar amount of an active moiety of the alkylating agent. In some embodiments, the amount (as assessed based on the final concentration in the sample containing solution) of the neutralizing agent is about 1 mM to about 100 mM.

In some embodiments, the alkylation technique, in whole or in part, is performed substantially in a low light condition. In some embodiments, the alkylation incubation time is performed in a low light condition. In some embodiments, the low light condition is in the dark or a location substantially devoid of sunlight and/or room lighting, such as in a desk drawer. In some embodiments, the low light condition is a filtered light, such as red light.

In some embodiments, the alkylating agent is sourced from a stock solution. In some embodiments, the stock solution is prepared within about 1 hour, such as within about any of 50 minutes, 40 minutes, 30 minutes, 20 minutes, or 10 minutes, of use.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

### IV. Proteolytic Digestion Techniques

In certain aspects, the methods provided herein comprise performing a proteolytic digestion technique. In some embodiments, the proteolytic digestion technique is performed on a sample, or a derivative thereof, following thermal denaturation and/or any additional steps intended to expose protease cleavage sites. Proteolytic digestion techniques, generally speaking, cleave polypeptides at known cleavage sites. For example, trypsin is a serine protease that generally cleaves polypeptides at the carboxyl side (C-terminal side) of lysine and arginine residues. Certain exceptions apply to the cleavage pattern of trypsin, such as due to proximity of a proline residue and/or a post-translational modification causing steric hindrance relative to the cleavage site. Proteolytic digestion techniques described herein comprise subjecting a sample, or a derivative thereof (e.g., a denatured sample or an alkylated sample, including an alkylated sample subjected to a reduction technique prior to an alkylation technique), to an amount of one or more proteases and incubating for a digestion incubation time performed at a temperature or range thereof.

In some embodiments, each of the one or more proteases is trypsin, LysC, LysN, AspN, GluC, ArgC, IdeS, IdeZ, PNGase F, thermolysin, pepsin, elastase, TEV, or Factor Xa, or any mixture thereof. In some embodiments, wherein two or more proteases are used, the weight ratio between a first protease and a second protease is about 1:10 to about 10:1, such as about any of about 1:9, 1:8, 1:7: 1:6, 1:5, 1:4, 1:3, 1:2, or 1:1. In some embodiments, the one or more proteases is trypsin. In some embodiments, the one or more proteases is a mixture of trypsin and LysC, such as in a weight ratio of about 1:1.

In some embodiments, the one or more proteases and amounts thereof are selected based on the type and/or characteristic of a biological sample and/or fibrinogen-depleted sample, or a derivative thereof, used in the methods herein. In some embodiments, the method comprises subjecting a fibrinogen-depleted sample, or a derivative thereof, to one or more proteases are present at a weight ratio of about 1:30 or less, relative to polypeptide content of the fibrinogen-depleted sample, or a derivative thereof. In some embodiments, the one or more proteases are added at a weight ratio of about 1:15 to about 1:25, relative to polypeptide content of the fibrinogen-depleted sample, or the derivative thereof. As described herein, the fibrinogen-depleted samples have lower complexity, such as compared to a plasma sample, and in some embodiments the method comprise a proteolytic digestion technique only using a single protease type (e.g., trypsin).

In some embodiments, the fibrinogen-depleted sample, or a derivative thereof, is digested using trypsin. In some embodiments, the protease, such as trypsin, is a modified protease, such as comprising a modification to prevent or inhibit self-proteolysis. In some embodiments, the modified protease is a modified trypsin, such as a methylated and/or an acetylated trypsin. In some embodiments, the modified trypsin is a tosyl phenylalanyl chloromethyl ketone (TPCK)-treated trypsin.

In some embodiments, the amount of a protease, e.g., trypsin or LysC, used in a proteolytic digestion technique is based on a weight ratio relative to the polypeptide content of a sample, or a derivative thereof, (i.e., weight of a protease: weight of polypeptide content) of about 1:200 to about 1:10, such as any of about 1:100 to about 1:10, about 1:50 to about 1:10, about 1:40 to about 1:20, about 1:50 to about 1:30, about 1:45 to about 1:35, about 1:20 to about 1:40, about 1:30 to about 1:10, or about 1:25 to about 1:15. In some embodiments, the amount of a protease used in a proteolytic digestion technique is at least about 1:200, such as at least about any of 1:190, 1:180, 1:170, 1:160, 1:150, 1:140, 1:130, 1:120, 1:110, 1:100, 1:95, 1:90, 1:85, 1:80, 1:75, 1:70, 1:65, 1:60, 1:55, 1:50, 1:45, 1:40, 1:35, 1:30, 1:25, 1:20, 1:15, or 1:10. In some embodiments, the amount of a protease used in a proteolytic digestion technique is about 1:10 or less, such as about any of 1:15 or less, 1:20 or less, 1:25 or less, 1:30 or less, 1:35 or less, 1:40 or less, 1:45 or less, 1:50 or less, 1:55 or less, 1:60 or less, 1:65 or less, 1:70 or less, 1:75 or less, 1:80 or less, 1:85 or less, 1:90 or less, 1:95 or less, 1:100 or less, 1:110 or less, 1:120 or less, 1:130 or less, 1:140 or less, 1:150 or less, 1:160 or less, 1:170 or less, 1:180 or less, 1:190 or less, or 1:200 or less. In some embodiments, the amount of a protease used in a proteolytic digestion technique is about any of 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, 1:110, 1:120, 1:130, 1:140, 1:150, 1:160, 1:170, 1:180, 1:190, or 1:200. In some embodiments, the proteolytic digestion technique comprises the use of two or more proteases, such as a combination of trypsin and LysC, and in such embodiments, the amount of each protease (such as described above) can be summed to a total amount of proteases used in a proteolytic digestion technique.

In some embodiments, the proteolytic digestion incubation time is about 20 minutes to about 36 hours, such as any of about 1 hour to about 18 hours, about 5 hours to about 24 hours, about 12 hours to about 24 hours, about 16 hours to about 20 hours, or about 12 hours to about 36 hours. In some embodiments, the proteolytic digestion incubation time is about 36 hours or less, such as about any of 32 hours or less, 30 hours or less, 28 hours or less, 26 hours or less, 24 hours or less, 22 hours or less, 20 hours or less, 19 hours or less, 18 hours or less, 17 hours or less, 16 hours or less, 15 hours or less, 14 hours or less, 13 hours or less, 12 hours or less, 11 hours or less, 10 hours or less, 9 hours or less, 8 hours or less, 7 hours or less, 6 hours or less, 5 hours or less, 4 hours or less, 3 hours or less, 2 hours or less, or 1 hours or less. In some embodiments, the proteolytic digestion incubation time is at least about 20 minutes, such as at least about any of 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 22 hours, 24 hours, 26 hours, 28 hours, 30 hours, 32 hours, 34 hours, or 36 hours. In some embodiments, the proteolytic digestion incubation time is about any of 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 22 hours, 24 hours, 26 hours, 28 hours, 30 hours, 32 hours, 34 hours, or 36 hours.

In some embodiments, the digestion incubation time is performed at a temperature of about 20° C. to about 60° C., such as any of about 20° C. to about 25° C., about 20° C. to about 30° C., about 25° C. to about 40° C., about 35° C. to about 40° C., or about 35° C. to about 50° C. In some embodiments, the digestion incubation time is performed at a temperature of at least about 20° C., such as at least about any of 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 42° C., 44° C., 46° C., 48° C., 50° C., 52° C., 54° C., 56° C., 58° C., or 60° C. In some embodiments, the digestion incubation time is performed at a temperature of about 60° C. or less, such about any of 58° C. or less, 56° C. or less, 54° C. or less, 52° C. or less, 50° C. or less, 48° C. or less, 46° C. or less, 44° C. or less, 42° C. or less, 40° C. or less, 39° C. or less, 38° C. or less, 37° C. or less, 36° C. or less, 35° C. or less, 34° C. or less, 33° C. or less, 32° C. or less, 31° C. or less, 30° C. or less, 29° C. or less, 28° C. or less, 27° C. or less, 26° C. or less, 25° C. or less, 24° C. or less, 23° C. or less, 22° C. or less, 21° C. or less, or 20° C. or less. In some embodiments, the digestion incubation time is performed at a temperature of about any of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 42° C., 44° C., 46° C., 48° C., 50° C., 52° C., 54° C., 56° C., 58° C., or 60° C. In some embodiments, the reduction incubation time is performed at a room temperature.

In some embodiments, the proteolytic digestion technique comprises a digestion incubation time of about 12 hours to about 24 hours, such as about any of 13 hours, 14 hours, 15 hours, 16 hours, 17 hours, 18 hours, 19 hours, 20 hours, 21 hours, 22 hours, 23 hours, wherein the digestion incubation time is performed at a temperature of about 20° C. to about 40° C., such about any of 22° C., 24° C., 26° C., 28° C., 30° C., 31° C., 32° C., 33° C., 34° C., 35° C., 36° C., 37° C., 38° C., 39° C. In some embodiments, the proteolytic digestion technique comprises use of an amount a protease for each of one or more proteases, e.g., trypsin and/or LysC, of about 1:15 to about 1:45, such as about any of 1:20, 1:25, 1:30, 1:35, or 1:40 (as measured based on the amount of the protease to the amount of polypeptide in a sample or a derivative thereof), and a digestion incubation time of about 12 hours to about 24 hours, such as about any of 13 hours, 14 hours, 15 hours, 16 hours, 17 hours, 18 hours, 19 hours, 20 hours, 21 hours, 22 hours, 23 hours, wherein the digestion incubation time is performed at a temperature of about 20° C. to about 40° C., such about any of 22° C., 24° C., 26° C., 28° C., 30° C., 31° C., 32° C., 33° C., 34° C., 35° C., 36° C., 37° C., 38° C., 39° C. In some embodiments, the proteolytic digestion technique is performed on a plasma sample or a derivate thereof, wherein the proteolytic digestion technique comprises a 1:40 ratio of trypsin to polypeptide in the sample of the derivative thereof and a 1:40 ratio of LysC to polypeptide in the sample or the derivative thereof. In some embodiments, the proteolytic digestion technique is performed on a serum sample or a derivate thereof, wherein the proteolytic digestion technique comprises a 1:20 ratio of trypsin.

The digestion incubation temperature can be controlled by numerous techniques and combinations thereof. In some embodiments, the digestion incubation temperature is controlled by an ambient temperature, such as room temperature. In some embodiments, the digestion incubation temperature is controlled by a water bath. In some embodiments, the digestion incubation temperature is controlled by a heat block. In some embodiments, the digestion incubation temperature is controlled by a thermocycler, e.g., a thermocycler with a lid temperature control element. As described herein, in some embodiments, control of sample temperature when performed using a thermocycler is via a temperature block element. In some embodiments, temperature changes prior to and/or after the digestion incubation time temperature are controlled by a technique described herein, such as cooling at room temperature or a ramp rate.

In some embodiments, the proteolytic digestion technique comprises subjecting a sample, or a derivative thereof, e.g., an alkylated sample (including an alkylated sample quenched with a neutralizing agent), to a thermal cycle. In some embodiments, the thermal cycle comprises subjecting the sample, or a derivative thereof, to one or more of: (a) a block starting temperature (b) block set temperature (the temperature for the digestion incubation time); (c) a block ending temperature; (d) one or more ramp rates between temperature changes in the thermal cycle (such as between the block starting temperature and the block set temperature or between the block set temperature and the block ending temperature); and (e) a lid temperature relative to the block temperature. In some embodiments, the thermal cycle is performed, in whole or in part, using a thermocycler. In some embodiments, the thermal cycle is configured to reduce and/or prevent loss of sample, such as by escaping vapor and/or condensation when the sample container is opened. In some embodiments, the thermal cycle comprises a set block temperature of about 20° C. to about 50° C., including about any of 22° C., 24° C., 26° C., 28° C., 30° C., 31° C., 32° C., 33° C., 34° C., 35° C., 36° C., 37° C., 38° C., 39° C., 40° C., 42° C., 44° C., 46° C., 48° C.

In some embodiments, the thermal cycle comprises: (a) a set block temperature of about 20° C. to about 50° C., including about any of 22° C., 24° C., 26° C., 28° C., 30° C., 31° C., 32° C., 33° C., 34° C., 35° C., 36° C., 37° C., 38° C., 39° C., 40° C., 42° C., 44° C., 46° C., 48° C., and (b) a block ending temperature of about 15° C. to about 35° C., such as any of about 20° C. to about 35° C., or about 20° C. to about 25° C. In some embodiments, the thermal cycle comprises: (a) starting block temperature of about 15° C. to about 35° C., such as any of about 20° C. to about 35° C., or about 20° C. to about 25° C., (b) a set block temperature of about 20° C. to about 50° C., including about any of 22° C., 24° C., 26° C., 28° C., 30° C., 31° C., 32° C., 33° C., 34° C., 35° C., 36° C., 37° C., 38° C., 39° C., 40° C., 42° C., 44° C., 46° C., 48° C., and (c) a block ending temperature of about 15° C. to about 35° C., such as any of about 20° C. to about 35° C., or about 20° C. to about 25° C. In any of the thermal cycles described herein, in some embodiments, the lid temperature during the thermal cycle is configured to reduce and/or inhibit condensate formation near or on the lid of a sample container. In some embodiments, the lid temperature during the thermal cycle is at least about 2° C., such as at least about any of 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 11° C., 12° C., 13° C., 14° C., 15° C., 16° C., 17° C., 18° C., 19° C., or 20° C., higher than the respective temperature of the block during the thermal cycle. In some embodiments, the lid temperature during at least a portion of a thermal cycle is about 102° C. to about 120° C., such as about any of 103° C., 104° C., 105° C., 106° C., 107° C., 108° C., 109° C., or 110° C.

In some embodiments, the ramp rate between a temperature change in a thermal cycle (such as between a block starting temperature and a block set temperature and/or between a block set temperature a the block ending temperature) is about 1° C./second to about 10° C./second, such as any of 1° C./second, 1.5° C./second, 2° C./second, 2.5° C./second, 3° C./second, 3.5° C./second, 4° C./second, 4.5° C./second, 5° C./second, 5.5° C./second, 6° C./second, 6.5° C./second, 7° C./second, 7.5° C./second, 8° C./second, 8.5° C./second, 9° C./second, 9.5° C./second, or 10° C./second.

In some embodiments, the proteolytic digestion technique further comprises quenching the one or more proteolytic enzymes. In some embodiments, quenching the one or more proteolytic enzymes comprises denaturing the one or more proteolytic enzymes. In some embodiments, quenching the one or more proteolytic enzymes comprise adding an amount of an acid. In some embodiments, the acid is formic acid (FA) or trifluoroacetic acid (TFA), or a mixture thereof. In some embodiments, the amount (as assessed based on the final concentration in the sample containing solution) of the acid added is about any of 0.1% v/v, 0.2% v/v, 0.3% v/v, 0.4% v/v, 0.5% v/v, 0.6% v/v, 0.7% v/v, 0.8% v/v, 0.9% v/v, 1% v/v, 1.1% v/v, 1.2% v/v, 1.3% v/v, 1.4% v/v, 1.5% v/v, 1.6% v/v, 1.7% v/v, 1.8% v/v, 1.9% v/v, or 2% v/v.

### V. Additional Techniques

In certain aspects, the method provided herein comprise subjecting the proteolytic digest sample comprising a proteolytic glycopeptide to one or more additional steps prior to subjecting the proteolytic digested sample, or a derivative thereof, to a liquid chromatography-mass spectrometry (LC-MS) technique using a liquid chromatography system and a mass spectrometer. In some embodiments, the LC system is online with the MS (i.e., eluate from the LC system is directly introduced to the MS).

In some embodiments, the method further comprises adding a standard to the proteolytic digest sample prior to the LC-MS technique. In some embodiments, the standard is a stable isotope-internal standard (SI-IS) peptide mixture.

In some embodiments, the fibrinogen-depleted sample, or a derivative thereof, is not subjected to a high-abundant protein depletion technique (other than the fibrinogen-depletion methods taught herein) prior to the thermal denaturation technique. For example, in some embodiments, the high-abundant protein depletion technique removes highly abundant proteins present in a blood sample, such as serum albumin.

### E6. Methods for Processing a Proteolytic Digest Sample

### I. Reversed-Phase (RP)-Based Techniques

Provided herein, in certain aspects, are methods of processing a proteolytically digested sample using a solid phase extraction column comprising a reversed-phase material. As described in the instant application, in some embodiments, the techniques taught herein for subjecting a proteolytically digested sample to the solid phase extraction column and/or subjecting the reversed-phase medium comprising associated proteolytic polypeptides to a wash buffer provide improved LC-MS analyses of proteolytic glycopeptides. In some embodiments, the method comprises subjecting a proteolytically digested sample to a solid phase extraction column comprising a reversed-phase medium to associate at least a portion of a plurality of proteolytic polypeptides with the reversed-phase medium, wherein the polypeptide loading amount used for subjecting the reversed-phase medium to the portion of the plurality of proteolytic polypeptides is about 50% or less of a binding capacity of the reversed-phase medium, and wherein the binding capacity of the reversed-phase medium is based on an insulin load having 10% or less breakthrough. In some embodiments, the method comprises subjecting a proteolytically digested sample to a solid phase extraction column comprising a reversed-phase medium to associate at least a portion of a plurality of proteolytic polypeptides with the reversed-phase medium, wherein the polypeptide loading concentration used for subjecting the proteolytic polypeptides to the reversed-phase medium is about 0.6 μg/μL or less. In some embodiments, the method comprises subjecting a reversed-phase medium comprising the associated proteolytic polypeptides to a wash buffer at a wash flow rate of about 0.1 column volumes/minute to about 2 column volumes/minute.

In the following sections, additional description of the various aspects of the methods for processing a proteolytically digested sample is provided. Such description in a modular fashion is not intended to limit the scope of the disclosure, and based on the teachings provided herein one of ordinary skill in the art will readily appreciate that certain modules can be integrated, at least in part. The section heading used herein are for organizational purposes only and are not to be construed as limiting the subject matter described.

a. Polypeptide Loading Amounts

In certain aspects, provided herein is a method for processing a proteolytically digested sample to produce a processed sample suitable for use in a liquid chromatography-mass spectrometry (LC-MS) analysis, wherein the method comprises subjecting the proteolytically digested sample to a solid phase extraction column comprising a reversed-phase medium according to a desired polypeptide loading amount based on the binding capacity of the reversed-phase medium. In some embodiments, the method comprises subjecting a proteolytically digested sample to a solid phase extraction column comprising a reversed-phase medium to associate at least a portion of a plurality of proteolytic polypeptides with the reversed-phase medium, wherein the polypeptide loading amount used for subjecting the reversed-phase medium to the portion of the plurality of proteolytic polypeptides is 50% or less of a binding capacity of the reversed-phase medium, and wherein the binding capacity of the reversed-phase medium is based on an insulin load having 10% or less breakthrough.

The polypeptide loading amounts encompassed herein may be described using a number of approaches, e.g., a percentage of the total binding capacity as defined by a known relevant binding capacity of a solid phase extraction column, or an absolute amount of polypeptide loaded onto a solid phase extraction column. One of ordinary skill in the art will readily understand converting between different forms of the description provided herein, and determining a binding capacity of a solid phase extraction column if not already known.

In some embodiments, the polypeptide loading amount is about 1% to about 50%, such as any of about 5% to about 50%, about 7.5% to about 50%, about 7.5% to about 25%, about 15% to about 50%, about 15% to about 25%, of a binding capacity of a reversed-phase medium of a solid phase extraction column. In some embodiments, the polypeptide loading amount is about 50% or less, such as any of 45% or less, 40% or less, 35% or less, 30% or less, 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, 10% or less, 9.5% or less, 9% or less, 8.5% or less, 8% or less, or 7.5% or less, of a binding capacity of a reversed-phase medium of a solid phase extraction column. In some embodiments, the polypeptide loading amount is about 7.5%, 8%, 8.5%, 9%, 9.5%, 10%, 11%, 12%, 13%, 14%, 15%, 16%, 17%, 18%, 19%, 20%, 21%, 22%, 23%, 24%, 25%, 30%, 35%, 40%, 45%, or 50%, of a binding capacity of a reversed-phase medium of a solid phase extraction column. In any of the embodiments above, the binding capacity of the reversed-phase medium is based on an insulin load having 10% or less breakthrough. In any of the embodiments above, the binding capacity of the reversed-phase medium of the solid phase extraction column is about 400 μg.

In some embodiments, the polypeptide loading amount is about 1% to about 50%, such as any of about 5% to about 50%, about 7.5% to about 50%, about 7.5% to about 25%, about 15% to about 50%, about 15% to about 25%, of a binding capacity of a reversed-phase medium of a solid phase extraction column, wherein the binding capacity of the reversed-phase medium of the solid phase extraction column is about 400 μg. In some embodiments, the polypeptide loading amount is about 50% or less, such as any of 45% or less, 40% or less, 35% or less, 30% or less, 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, 10% or less, 9.5% or less, 9% or less, 8.5% or less, 8% or less, or 7.5% or less, of a binding capacity of a reversed-phase medium of a solid phase extraction column, wherein the binding capacity of the reversed-phase medium of the solid phase extraction column is about 400 μg. In some embodiments, the polypeptide loading amount is about 7.5%, 8%, 8.5%, 9%, 9.5%, 10%, 11%, 12%, 13%, 14%, 15%, 16%, 17%, 18%, 19%, 20%, 21%, 22%, 23%, 24%, 25%, 30%, 35%, 40%, 45%, or 50%, of a binding capacity of a reversed-phase medium of a solid phase extraction column, wherein the binding capacity of the reversed-phase medium of the solid phase extraction column is about 400 μg. In any of the embodiments above, the binding capacity of the reversed-phase medium is based on an insulin load having 10% or less breakthrough.

In some embodiments, the polypeptide loading amount is about 30 μg to about 200 μg, such as any of about 30 μg to about 100 μg, about 30 μg to about 60 μg to about 200 μg, or about 60 μg to about 100 μg, wherein the binding capacity of the reversed-phase medium of the solid phase extraction column is about 400 μg. In some embodiments, the polypeptide loading amount is about 200 μg or less, such as any of 175 μg or less, 150 μg or less, 125 μg or less, 100 μg or less, 95 μg or less, 90 μg or less, 85 μg or less, 80 μg or less, 75 μg or less, 70 μg or less, 65 μg or less, 60 μg or less, 55 μg or less, 50 μg or less, 45 μg or less, 40 μg or less, 35 μg or less, or 30 μg or less, wherein the binding capacity of the reversed-phase medium of the solid phase extraction column is about 400 μg. In some embodiments, the polypeptide loading amount about any of 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, or 200 μg, wherein the binding capacity of the reversed-phase medium of the solid phase extraction column is about 400 μg. In any of the embodiments above, the binding capacity of the reversed-phase medium is based on an insulin load having 10% or less breakthrough.

In some embodiments, the polypeptide loading amount is about 30 μg to about 200 μg, such as any of about 30 μg to about 100 μg, about 30 μg to about 60 μg to about 200 μg, or about 60 μg to about 100 μg, wherein the solid phase extraction column comprises a volume of about 5 μL of a reversed-phase medium. In some embodiments, the polypeptide loading amount is about 200 μg or less, such as any of 175 μg or less, 150 μg or less, 125 μg or less, 100 μg or less, 95 μg or less, 90 μg or less, 85 μg or less, 80 μg or less, 75 μg or less, 70 μg or less, 65 μg or less, 60 μg or less, 55 μg or less, 50 μg or less, 45 μg or less, 40 μg or less, 35 μg or less, or 30 μg or less, wherein the solid phase extraction column comprises a volume of about 5 μL of a reversed-phase medium. In some embodiments, the polypeptide loading amount about any of 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, or 200 μg, wherein the solid phase extraction column comprises a volume of about 5 μL of a reversed-phase medium. In any of the embodiments above, the binding capacity of the reversed-phase medium of the solid phase extraction column is about 400 μg. In any of the embodiments above, the binding capacity of the reversed-phase medium is based on an insulin load having 10% or less breakthrough.

The solid phase extraction columns described herein may comprise a reversed-phase medium, or an amount thereof, encompassing a range of binding capacities for the solid phase extraction column. In some embodiments, the solid phase extraction column comprises a binding capacity of about 1 μg to about 1,000 μg, such as any of about 100 μg to about 500 μg, about 200 μg to about 500 μg, about 300 μg to about 500 μg, about 350 μg to about 500 μg, or about 350 μg to about 750 μg, such as assessed using a polypeptide or a mixture thereof, including insulin. In some embodiments, the binding capacity is based on the amount of a polypeptide (including mixtures of polypeptides) that can be associated with the reversed-phase medium of a solid phase extraction column prior to occurrence of breakthrough (loss of polypeptides in the load that occurs during a binding phase such that a portion of the polypeptide is not captured by the reversed-phase medium) 10% or more. In some embodiments, the binding capacity of the reversed-phase medium is based on a polypeptide load having 10% or less, such as any of 9% or less, 8% or less, 7% or less, 6% or less, 5% or less, 4% or less, 3% or less, 2% or less, or 1% or less, breakthrough.

Polypeptide amounts described herein may be absolute or estimated amounts. In some embodiments, the amount of polypeptide content in a sample, or a derivative thereof, is a measured directly from said sample, or the derivative thereof, e.g., using a BCA quantification assay or a UV-VIS measurement at 280 nm. In some embodiments, the amount of polypeptide content in a sample, or a derivative thereof, is estimated based on a known, including reference standard, value for polypeptide content in the sample based on the origin of the sample, e.g., such as based on a known standard polypeptide concentration in human plasma or serum.

b. Polypeptide Loading Concentrations

In certain aspects, provided herein is a method for processing a proteolytically digested sample to produce a processed sample suitable for use in a liquid chromatography-mass spectrometry (LC-MS) analysis, wherein the method comprises subjecting the proteolytically digested sample to a solid phase extraction column comprising a reversed-phase medium according to a desired polypeptide loading concentration. In some embodiments, the method comprises subjecting a proteolytically digested sample to a solid phase extraction column comprising a reversed-phase medium to associate at least a portion of a plurality of proteolytic polypeptides with the reversed-phase medium, wherein the polypeptide loading concentration used for subjecting the proteolytic polypeptides to the reversed-phase medium is about 0.6 μg/μL or less.

In some embodiments, the polypeptide loading concentration used for subjecting polypeptides of the proteolytically digested sample to a reversed-phase medium of a solid phase extraction column is about 0.1 μg/μL to about 1 μg/μL, such as any of about 0.25 μg/μL to about 1 μg/μL, about 0.25 μg/μL to about 0.75 μg/μL, or about 0.3 μg/μL to about 0.6 μg/μL. In some embodiments, the polypeptide loading concentration used for subjecting polypeptides of the proteolytically digested sample to a reversed-phase medium of a solid phase extraction column is about 1 μg/μL or less, such as about any of 0.95 μg/μL or less, 0.9 μg/μL or less, 0.85 μg/μL or less, 0.8 μg/μL or less, 0.75 μg/μL or less, 0.7 μg/μL or less, 0.65 μg/μL or less, 0.6 μg/μL or less, 0.55 μg/μL or less, 0.5 μg/μL or less, 0.45 μg/μL or less, 0.4 μg/μL or less, 0.35 μg/μL or less, or 0.3 μg/μL or less. In some embodiments, the polypeptide loading concentration used for subjecting polypeptides of the proteolytically digested sample to a reversed-phase medium of a solid phase extraction column is about any of 1 μg/μL 0.95 μg/μL, 0.9 μg/μL, 0.85 μg/μL, 0.8 μg/μL, 0.75 μg/μL, 0.7 μg/μL, 0.65 μg/μL, 0.6 μg/μL, 0.55 μg/μL, 0.5 μg/μL, 0.45 μg/μL, 0.4 μg/μL, 0.35 μg/μL, or 0.3 μg/μL.

In some embodiments, the polypeptides of the proteolytically digested sample for subjecting to the solid phase extraction column are in a volume of about 50 μL to about 500 μL, such as any of about 50 μL to about 300 μL, about 50 μL to about 250 μL, or about 100 μL to about 200 μL. In some embodiments, the polypeptides of the proteolytically digested sample for subjecting to the solid phase extraction column are in a volume of at least about 50 μL, such as at least about any of 75 μL, 100 μL, 125 μL, 150 μL, 175 μL, 200 μL, 225 μL, 250 μL, 275 μL, 300 μL, 325 μL, 350 μL, 375 μL, 400 μL, 425 μL, 450 μL, 475 μL, or 500 μL. In some embodiments, the polypeptides of the proteolytically digested sample for subjecting to the solid phase extraction column are in a volume that is at least about 40%, such as at least about any of 45%, 50%, 55%, 60%, 65%, 70%, 75%, 80%, 85%, 90%, or 95%, of the upper range recommended for a solid phase extraction column, such as per manufacturer's instructions. In some embodiments, the volume loaded onto the solid phase extraction column comprises a loading buffer.

As discussed herein, polypeptide amounts described herein may be absolute or estimated amounts. In some embodiments, the amount of polypeptide content in a sample, or a derivative thereof, is a measured directly from said sample, or the derivative thereof, e.g., using a BCA quantification assay. In some embodiments, the amount of polypeptide content in a sample, or a derivative thereof, is estimated based on a known, including reference standard, value for polypeptide content in the sample based on the origin of the sample, e.g., such as based on a known standard polypeptide concentration in human plasma or serum.

c. Wash Buffer Flow Rates

In certain aspects, provided herein is a method for processing a proteolytically digested sample to produce a processed sample suitable for use in a liquid chromatography-mass spectrometry (LC-MS) analysis, wherein the method comprises subjecting a reversed-phase medium comprising the associated proteolytic polypeptides to a wash buffer at a desired wash flow rate. In some embodiments, the method comprises subjecting a reversed-phase medium comprising the associated proteolytic polypeptides to a wash buffer at a wash flow rate of about 0.1 column volumes/minute to about 2 column volumes/minute.

In some embodiments, the wash flow rate of the wash buffer is about 0.1 column volumes/minute to about 2 column volumes/minute, such as any of about 0.1 column volumes/minute to about 0.5 column volumes/minute, about 0.25 column volumes/minute to about 2 column volumes/minute, about 0.25 column volumes/minute to about 1.5 column volumes/minute, or about 0.3 column volumes/minute to about 1 column volumes/minute. In some embodiments, the wash flow rate of the wash buffer is less than about 2, such as less than about any of 1.9 column volumes/minute, 1.8 column volumes/minute, 1.7 column volumes/minute, 1.6 column volumes/minute, 1.5 column volumes/minute, 1.4 column volumes/minute, 1.3 column volumes/minute, 1.2 column volumes/minute, 1.1 column volumes/minute, 1 column volume/minute, 0.9 column volumes/minute, 0.8 column volumes/minute, 0.7 column volumes/minute, 0.6 column volumes/minute, 0.5 column volumes/minute, 0.4 column volumes/minute, 0.3 column volumes/minute or 0.2 column volumes/minute. In some embodiments, the wash flow rate of the wash buffer is about any of 0.1 column volumes/minute, 0.2 column volumes/minute, 0.3 column volumes/minute, 0.4 column volumes/minute, 0.5 column volumes/minute, 0.6 column volumes/minute, 0.7 column volumes/minute, 0.8 column volumes/minute, 0.9 column volumes/minute, 1 column volume/minute, 1.1 column volumes/minute, 1.2 column volumes/minute, 1.3 column volumes/minute, 1.4 column volumes/minute, 1.5 column volumes/minute, 1.6 column volumes/minute, 1.7 column volumes/minute, 1.8 column volumes/minute, 1.9 column volumes/minute, or 2 column volumes/minute. In some embodiments, the column volume refers to the volume occupied by the reversed-phase media within the solid phase extraction column or cartridge.

In some embodiments, the wash flow rate of the wash buffer is about 1 μL/minute to about 10 μL/minute, such as about 2 μL/minute to about 8 μL/minute, about 2 μL/minute to about 5 μL/minute, or about 2 μL/minute to about 4 μL/minute. In some embodiments, the wash flow rate of the wash buffer is about 10 L/minute or less, such as any of about 9 μL/minute or less, 8 μL/minute or less, 7 μL/minute or less, 6 μL/minute or less, 5 μL/minute or less, 4 μL/minute or less, 3 μL/minute or less, 2 μL/minute or less, or 1 μL/minute or less. In some embodiments, the wash flow rate of the wash buffer is about any of 1 μL/minute, 2 μL/minute, 3 μL/minute, 4 μL/minute, 5 μL/minute, 6 μL/minute, 7 μL/minute, 8 μL/minute, 9 μL/minute, or 10 μL/minute. In any of the embodiments above, the column volume is about 5 μL.

In some embodiments, the method comprises subjecting the reversed-phased medium comprising the associated proteolytic polypeptides to a wash buffer, wherein the total wash buffer applied to the reversed-phase medium is about 1 to about 50 column volumes. In some embodiments, the total wash buffer applied to the reversed-phase medium is about 50 or fewer, such as any of 45 or fewer, 40 or fewer, 35 or fewer, 30 or fewer, 25 or fewer, 20 or fewer, 15 or fewer, 10 or fewer, or 5 or fewer, column volumes.

d. Reversed-Phase Mediums of Solid Phase Extraction Columns

The methods provided herein, in certain aspects, involve solid phase extraction columns comprising a reversed-phase medium. Generally speaking, reversed-phase media comprise a relatively hydrophobic stationary phase configured to associate with proteolytic polypeptides, wherein relatively hydrophilic compounds, such as salts, reagents, or byproducts thereof present in a proteolytically digested sample, can be eluted from the reversed-phase medium prior to the proteolytic polypeptides using an aqueous mobile phase.

In some embodiments, the reversed-phase medium comprises an alkyl-based moiety covalently bound to a solid phase. In some embodiments, the alkyl-based moiety comprises an alkyl carbon functional group having between 1 carbon and 30 carbons, such as any of 4 carbons to 18 carbons, 8 carbons to 18 carbons, or 18 carbons to 30 carbons. In some embodiments, the alkyl-based moiety comprises an alkyl carbon functional group having 30 or fewer carbons, such as any of 25 or fewer carbons, 20 or fewer carbons, 19 or fewer carbons, 18 or fewer carbons, 17 or fewer carbons, 16 or fewer carbons, 15 or fewer carbons, 14 or fewer carbons, 13 or fewer carbons, 12 or fewer carbons, 11 or fewer carbons, 10 or fewer carbons, 9 or fewer carbons, 8 or fewer carbons, 7 or fewer carbons, 6 or fewer carbons, 5 or fewer carbons, 4 or fewer carbons. In some embodiments, the alkyl-based moiety comprises an alkyl carbon functional group comprising 4 carbons, 5 carbons, 6 carbons, 7 carbons, 8 carbons, 9 carbons, 10 carbons, 11 carbons, 12 carbons, 13 carbons, 14 carbons, 15 carbons, 16 carbons, 17 carbons, 18 carbons, 19 carbons, 20 carbons, 21 carbons, 22 carbons, 23 carbons, 24 carbons, 25 carbons, 26 carbons, 27 carbons, 28 carbons, 29 carbons, or 30 carbons. In some embodiments, the alkyl-based moiety comprises an octadecyl carbon functional group (C18) covalently bound to the solid phase. In some embodiments, the alkyl-based moiety comprises an octa carbon functional group (C8) covalently bound to the solid phase. In some embodiments, the carbon alkyl-based moiety comprises a tetra carbon functional group (C4) covalently bound to the solid phase.

In some embodiments, the reversed-phase medium comprises a silica-based material which supports an alkyl-based moiety. In some embodiments, the solid phase comprises a silica material. In some embodiments, the silica material is a silica gel, such as composed of a plurality of silica particles. In some embodiments, the silica-based material is inert. In some embodiments, the silica-based material is base-deactivated. In some embodiments, the silica-based material is an ultra-high purity silica material, such as an ultra-high purity silica gel. In some embodiments, the silica-based material comprises silanol groups that are partially or fully end-capped, such as, for example, with a C1 methyl group. In some embodiments, the silica-based material comprises silanol groups are not end-capped. The components of a silica-based material of a reversed-phase medium may take a diverse array of sizes and shapes. In some embodiments, the silica-based material comprises a plurality of particles, wherein the plurality of particles have an average largest cross-sectional distance (such as a diameter, e.g., as measured by dynamic light scattering) of about 0.5 μm to about 30 μm, such as any of about 1 μm to about 25 μm, about 15 μm to about 25 μm, about 18 μm to about 22 μm. In some embodiments, the silica-based material comprises a plurality of particles, wherein the plurality of particles have an average largest cross-sectional distance (such as a diameter, e.g., as measured by dynamic light scattering) of about any of 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, or 30 μm.

In some embodiments, the silica-based material comprises a plurality of particles, wherein each particle of the plurality of particles comprises an average pore size of about 1 Å to about 500 Å, such as about 50 Å to about 300 Å, or about 100 Å to about 200 Å. In some embodiments, the silica-based material comprises a plurality of particles, wherein each particle of the plurality of particles comprises an average pore size of about any of 5 Å, 10 Å, 20 Å, 25 Å, 30 Å, 35 Å, 40 Å, 45 Å, 50 Å, 60 Å, 70 Å, 80 Å, 90 Å, 100 Å, 110 Å, 120 Å, 130 Å, 140 Å, 150 Å, 160 Å, 170 Å, 180 Å, 190 Å, 200 Å, 225 Å, 250 Å, 275 Å, 300 Å, 325 Å, 350 Å, 375 Å, 400 Å, 425 Å, 450 Å, 475 Å, or 500 Å. In some embodiments, the pore size of the silica-based medium is uniform or substantially uniform. In some embodiments, the pore size of the silica-based medium is heterogeneous. In some embodiments, the pores of the silica-based medium are derivatized with an alkyl-based moiety.

In some embodiments, the reversed-phase medium comprises a hydrophobic polymer material (e.g., RP-S). In some embodiments, the hydrophobic polymer material comprises a phenyl moiety. In some embodiments, the hydrophobic polymer material comprises a reaction product of divinylbenzene. In some embodiments, the hydrophobic polymer material comprises poly(styrene-co-divinylbenzene). In some embodiments the reversed-phase medium comprising a hydrophobic polymer material is in the form a plurality of particles. The components of a hydrophobic polymer material (e.g., RP-S) reversed-phase medium may take a diverse array of sizes and shapes. In some embodiments, the hydrophobic polymer material comprises a plurality of particles, wherein the plurality of particles have an average largest cross-sectional distance (such as a diameter, e.g., as measured by dynamic light scattering) of about 0.5 μm to about 30 μm, such as any of about 1 μm to about 25 μm, about 15 μm to about 25 μm, about 18 μm to about 22 μm. In some embodiments, the hydrophobic polymer material comprises a plurality of particles, wherein the plurality of particles have an average largest cross-sectional distance (such as a diameter, e.g., as measured by dynamic light scattering) of about any of 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, or 30 μm. In some embodiments, the hydrophobic polymer material comprises a plurality of particles, wherein each particle of the plurality of particles comprises an average pore size of about 1 Å to about 500 Å, such as about 50 Å to about 300 Å, or about 100 Å to about 200 Å. In some embodiments, the hydrophobic polymer material comprises a plurality of particles, wherein each particle of the plurality of particles comprises an average pore size of about any of 5 Å, 10 Å, 20 Å, 25 Å, 30 Å, 35 Å, 40 Å, 45 Å, 50 Å, 60 Å, 70 Å, 80 Å, 90 Å, 100 Å, 110 Å, 120 Å, 130 Å, 140 Å, 150 Å, 160 Å, 170 Å, 180 Å, 190 Å, 200 Å, 225 Å, 250 Å, 275 Å, 300 Å, 325 Å, 350 Å, 375 Å, 400 Å, 425 Å, 450 Å, 475 Å, or 500 Å. In some embodiments, the pore size of the hydrophobic polymer material is uniform or substantially uniform. In some embodiments, the pore size of the hydrophobic polymer material is heterogeneous.

The reversed-phase media in the solid phase extraction columns may take a diverse array of forms. In some embodiments, the reversed-phase medium is in the form of a plurality of particles, wherein the solid phase extraction column comprises a packed column. In some embodiments, the solid phase extraction column comprises a surface modification forming the reversed-phase medium. In some embodiments, the solid phase extraction column comprises a monolithic structure. In some instances, the terms medium, media, and resin may be used interchangeably.

In some embodiments, the solid phase extraction column has a column volume of about 1 to about 10 μL, such as any of about 2 μL to about 8 μL, about 3 μL to about 7 μL, or about 4 μL to about 5 μL. In some embodiments, the solid phase extraction column has a column volume of about any of 1 μL, 2 μL, 3 μL, 4 μL, 5 μL, 6 μL, 7 μL, 8 μL, 9 μL, or 10 μL. The reversed-phase media can be referred to as a bed that is compacted within a chromatography column to form a bed volume.

In some embodiments, the reversed-phase medium comprises an alkyl-based moiety comprising an octadecyl carbon functional group (C18) covalently bound to a silica solid phase, wherein the silica solid phase comprises a plurality of particles having an average largest cross-sectional distance (such as a diameter, e.g., as measured by dynamic light scattering) of about 20 μm, and wherein the each particle of the plurality of particles comprises an average pore size of about 150 Å, and wherein the pores are derivatized with the alkyl-based moiety. In some embodiments, the solid phase extraction column comprising the reversed-phase medium has a column volume of about 5 μL. In some embodiments, the solid phase extraction column is an AssayMap 5 μL C18 cartridge (catalog no. 5190-6532; Agilent Technologies).

In some embodiments, the reversed-phase medium comprises an underivitized polystyrene divinylbenzene hydrophobic reversed-phase resin, wherein the polystyrene divinylbenzene hydrophobic reversed-phase resin comprises a plurality of particles having an average largest cross-sectional distance (such as a diameter, e.g., as measured by dynamic light scattering) of about 20 μm, and wherein each particle of the plurality of particles comprises an average pore size of about 100 Å. In some embodiments, the solid phase extraction column comprising the reversed-phase medium has a column volume of about 5 μL. In some embodiments, the solid phase extraction column is an AssayMAP 5 μL Reversed Phase (RP-S) cartridge (catalog no. G5496-60033; Agilent Technologies).

### II. HILIC Media-Based Techniques

Provided herein, in certain aspects, are methods of processing a proteolytic digest sample using a solid phase extraction column comprising a comprising a hydrophilic interaction liquid chromatography (HILIC) medium. As taught herein, the described methodology may be used for the enrichment of glycopolypeptides (e.g., a proteolytically digested glycopeptide), from a sample such as a proteolytic digest sample. In some embodiments, the resulting composition obtained from the methodology provided herein, or a derivative thereof, is suitable for use in a liquid chromatography-mass spectrometry (LC-MS) analysis, such as for the analysis of glycopolypeptides. In some embodiments, the enriched glycopeptide is a proteolytically digested glycopeptide. In some embodiments, the enriched glycopeptide is an endogenous glycopeptide. In some embodiments, the glycopeptide obtained from the method provided herein is enriched by a factor of at least 30 when compared to the polypeptide content in a respective proteolytic digest sample.

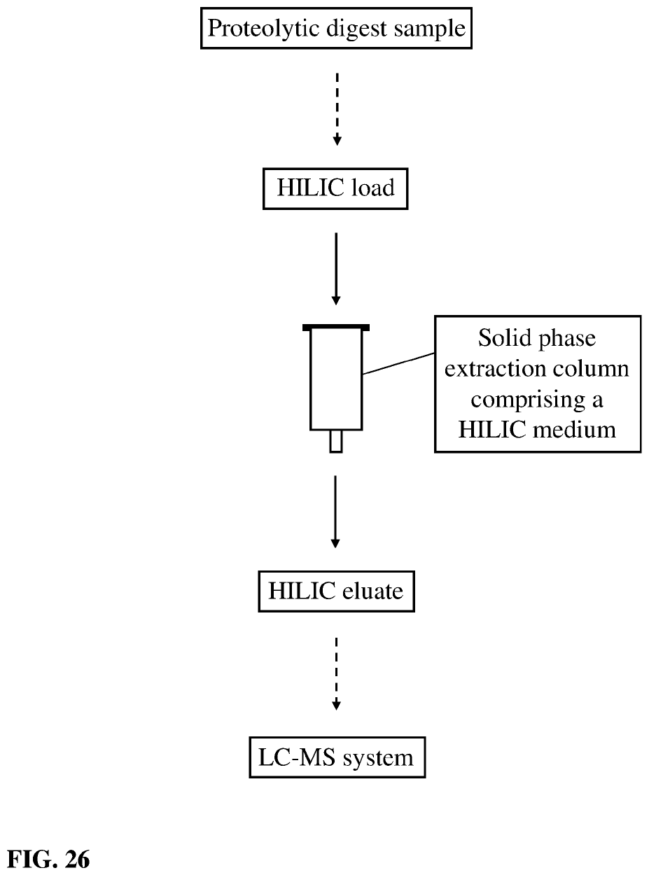

Aspects of the disclosure provided herein may be described via the state of the HILIC medium (e.g., dry state) when initiating loading of a HILIC load to a solid phase extraction column comprising a HILIC medium, and/or one or more conditions of the HILIC load, such as an amount of a plurality of proteolytically digested peptides relative to an amount of the HILIC medium in the solid phase extraction column and/or a concentration of an organic solvent of the HILIC load. In some embodiments, methodology is exemplified for obtaining the HILIC load having the one or more conditions. For purposes of orientation of aspects of the methodology provided herein, FIG. 26 illustrates an example workflow from a proteolytic digest sample to a LC-MS system. In some embodiments, the proteolytic digest sample resulting from a proteolytic digestion is not suitable for direct introduction to the LC-MS system (or it is not desirable to do so), such as due to conditions needed to facilitate reactions relevant to the proteolytic digestion (e.g., high salt and/or buffer content). As provided herein, in some embodiments, components of the proteolytic digest sample, such as a plurality of proteolytically digested peptides comprising at least one proteolytically digested glycopeptide, are subjected to a solid phase extraction column comprising a HILIC medium to associate the at least one proteolytically digested glycopeptide with the HILIC medium. The composition comprising the plurality of proteolytically digested peptides directly loaded onto the solid phase extraction column is referred to herein as a HILIC load (represented by the solid arrow in FIG. 26). In some embodiments, the methodology provided herein comprises loading a HILIC load having a specified condition (such as polypeptide amount relative to the amount of HILIC medium and/or concentration of an organic solvent) to a solid phase extraction column comprising a HILIC medium. In some embodiments, the proteolytic digest sample is subjected to one or more processing steps to form the HILIC load (represented by the dashed arrow in FIG. 26). As shown in FIG. 26, a HILIC eluate comprising the at least one glycopeptide is obtained from the solid phase extraction column. In some embodiments, the HILIC eluate is subjected to one or more processing steps in preparation for subjecting the at least one glycopeptide to the LC-MS system (represented by the dashed arrow in FIG. 26).

As described in certain aspects the instant application, in some embodiments, provided is a method for processing a proteolytic digest sample for use in a LC-MS analysis, wherein the proteolytic digest sample comprises a plurality of proteolytically digested peptides comprising at least one proteolytically digested glycopeptide, the method comprising loading a HILIC load derived from the proteolytic digest sample to a solid phase extraction column comprising a HILIC medium to associate the at least one proteolytically digested glycopeptide with the HILIC medium, wherein the loading of the HILIC load to the solid phase extraction column is initiated when the HILIC medium is in a dry state. As described in certain aspects the instant application, in some embodiments, provided is a method for processing a proteolytic digest sample for use in a LC-MS analysis, wherein the proteolytic digest sample comprises a plurality of proteolytically digested peptides comprising at least one proteolytically digested glycopeptide, the method comprising loading a HILIC load derived from the proteolytic digest sample to a solid phase extraction column comprising a HILIC medium to associate the at least one proteolytically digested glycopeptide with the HILIC medium, wherein the HILIC load loaded to the solid phase extraction column has an amount of the plurality of proteolytically digested peptides characterized by one or both of: (a) a ratio of the weight of the plurality of proteolytically digested peptides over the weight of the HILIC medium in the dry state of at least about 0.06; and/or (b) a ratio of the weight of the plurality of proteolytically digested peptides relative to the bed volume of the HILIC medium in the dry state of at least about 40 μg/μl. As described in certain aspects the instant application, in some embodiments, provided is a method for processing a proteolytic digest sample for use in a LC-MS analysis, wherein the proteolytic digest sample comprises a plurality of proteolytically digested peptides comprising at least one proteolytically digested glycopeptide, the method comprising loading a HILIC load derived from the proteolytic digest sample to a solid phase extraction column comprising a HILIC medium to associate the at least one proteolytically digested glycopeptide with the HILIC medium, wherein the HILIC load loaded to the solid phase extraction column has a concentration of an organic solvent of at least about 70% (v/v). As described in certain aspects the instant application, in some embodiments, provided is a method for processing a proteolytic digest sample for use in a LC-MS analysis, the method comprising any one or more combinations of the steps described herein.

In the following sections, additional description of the various aspects of the methods for processing a proteolytic digest sample is provided. Such description in a modular fashion is not intended to limit the scope of the disclosure, and based on the teachings provided herein one of ordinary skill in the art will readily appreciate that certain modules can be integrated, at least in part. The section heading used herein are for organizational purposes only and are not to be construed as limiting the subject matter described.

a. Dry State of HILIC Medium and HILIC Medium Characteristics

Provided herein, in some aspects, is a method for processing a proteolytic digest sample for use in a LC-MS analysis, the method comprising loading a HILIC load to a solid phase extraction column comprising a HILIC medium, wherein the loading of the HILIC load to the solid phase extraction column is initiated when the HILIC medium is in a dry state. One of ordinary skill in the art will readily appreciate that a HILIC medium will not remain in a dry state over the entire course of loading a HILIC load, i.e., the HILIC load will wet the HILIC medium over the course of applying the HILIC load to the HILIC medium. In some embodiments, the description of a dry state of a HILIC medium references the state of the HILIC medium immediately prior to a first portion of a HILIC load contacting the HILIC medium.

In some embodiments, the dry state of a HILIC medium is characterized by the HILIC medium comprising less than about 5% (v/v), such as less than about any of 4.5% (v/v), 4% (v/v), 3.5% (v/v), 3% (v/v), 2.5% (v/v), 2% (v/v), 1.5% (v/v), 1% (v/v), 0.5% (v/v), or 0.1% (v/v), of a liquid at the initiation of the loading of a HILIC load to a solid phase extraction column comprising the HILIC medium. In some embodiments, the HILIC medium is substantially dry, such as does contain a liquid in a measurable amount, such as above the level of liquid in surrounding air, e.g., as based on humidity. In some embodiments, the liquid comprises water.

In some embodiments, at the initiation of the loading of a HILIC load to a HILIC medium, the HILIC medium has not been subjected to a washing, equilibration, or wetting procedure. It is appreciated that in some embodiments, the HILIC medium may have been subjected to a liquid such as during manufacturing or packaging, and in certain aspects the present description regarding a dry state is focused on steps that occur after the HILIC medium is loaded into a cartridge to form the solid phase extraction column and dried (or a occur after a final drying step and prior to loading a HILIC load). For example, in some embodiments, the method comprises obtaining a solid phase extraction column comprising a HILIC medium, such as from a manufacturer, and loading a HILIC load to the solid phase extraction column without intervening steps of introducing a liquid to the solid phase extraction column, such as for purposes of a washing, equilibration, or wetting procedure. In some embodiments, at the initiation of the loading of a HILIC load to a HILIC medium, the HILIC medium is not in an equilibrated state. In some embodiments, at the initiation of the loading of a HILIC load to a HILIC medium, the HILIC medium is not in a wetted state. In some embodiments, at the initiation of the loading of a HILIC load to a HILIC medium, the HILIC medium is not in a swelled state. In some embodiments, at the initiation of the loading of a HILIC load to a HILIC medium, the HILIC medium is not in a hydrated state. In some embodiments, the HILIC medium may be subjected to a liquid, such as a washing liquid, and then subsequently dried, wherein at the initiation of the loading of a HILIC load to the HILIC medium, the HILIC load is in a dry state.

In some embodiments, at the initiation of the loading of a HILIC load on a HILIC medium, the HILIC medium is not equilibrated with an equilibration liquid. In some embodiments, at the initiation of the loading of a HILIC load on a HILIC medium, the HILIC medium is not washed with a wash liquid. In some embodiments, at the initiation of the loading of a HILIC load on a HILIC medium, the HILIC medium is not wetted with a wetting liquid. In some embodiments, during the loading of the HILIC load, the HILIC medium is hydrated with water and then binds to at least one glycopeptide, wherein the HILIC load includes both water and an organic miscible solvent.

In some embodiments, the method provided herein comprises drying a HILIC medium of a solid phase extraction column to obtain the HILIC medium in a dry state. In some embodiments, the drying comprises subjecting a solid phase extraction column, or a HILIC medium, to conditions such that a liquid in the solid phase extraction column of the HILIC medium can leave the solid phase extraction column. In some embodiments, the condition comprises a room temperature. In some embodiments, the condition comprises an elevate temperature. In some embodiments, the method comprises determining the liquid content of a HILIC medium to assess if it is in a dry state prior to loading a HILIC load to a solid phase extraction column comprising the HILIC medium. In some embodiments, drying a HILIC medium of a solid phase extraction column may comprises use of a force to push liquid through the solid phase extraction column, such as a centrifugal force, position pressure, and/or negative pressure.

In other aspects of the description provided herein, the HILIC medium need not be in a dry state at the initiation of the loading of the HILIC load to the solid phase extraction column. For example, in some embodiments, the solid phase extraction column is equilibrated, wetted, and/or washed prior to loading a HILIC load to the solid phase extraction column, such as according to manufacturer's instructions. In some embodiments, wherein a HILIC medium is not in a dry state, loading a HILIC load to a solid phase extraction column comprising the HILIC medium is characterized by one or more of: (1) the HILIC load loaded to the solid phase extraction column has an amount of the plurality of proteolytically digested peptides characterized by one or both of: (a) a ratio of the weight of the plurality of proteolytically digested peptides over the weight of the HILIC medium in the dry state of at least about 0.06; and/or (b) a ratio of the weight of the plurality of proteolytically digested peptides relative to the bed volume of the HILIC medium in the dry state of at least about 40 μg/μl; or (2) the HILIC load loaded to the solid phase extraction column has a concentration of an organic solvent of at least about 70% (v/v).

In some embodiments, the HILIC medium comprises a solid phase. In some embodiments, the HILIC medium comprises a solid phase comprising a polar functional moiety. In some embodiments, the solid phase comprises a silica material. In some embodiments, the polar functional moiety comprises one or more of an amino group, a cyano group, a carbamoyl group, an aminoalkyl group, alkylamide group, or a combination thereof. In some embodiments, the HILIC medium comprises an amide resin (e.g., Tosoh TSK-Gel Amide 80 Resin). In some embodiments, the HILIC medium comprises silica particles covalently bonded with carbamoyl groups. In some embodiments, the HILIC medium comprises 2 μm, 3 μm, 5 μm, or 10 μm silica particles of covalently bonded with carbamoyl groups, see, e.g., Y. Kawachi et al., J Chromatography A, 2011, which is incorporated herein by reference in its entirety. In some embodiments, the solid phase extraction column comprises about 0.5 mg to about 5 mg, including about any of 1 mg, 1.5 mg, 2 mg, 2.5 mg, 3 mg, 3.5 mg, 4 mg, or 4.5 mg, of a HILIC medium in a dry state. In some embodiments, the solid phase extraction column comprises a bed volume of a HILIC medium in a dry state of about 2.5 μl to about 7.5 μl, including about 5 μl. In some embodiments, the solid phase extraction column comprises about 2.5 mg to about 3.5 mg, including about 3 mg, of a HILIC medium in a dry state, a bed volume of the HILIC medium in a dry state of about 4 μl to about 6 μl, including about 5 μl, and wherein the HILIC medium comprises silica particles covalently bonded with carbamoyl groups. In some embodiments, the solid phase extraction column is a GlykoPrep Cleanup cartridge (e.g., Product code: GS96-CU; WS0263).

HILIC is used primarily for the separation of polar and hydrophilic compounds such as, for example, glycopeptides. HILIC stationary phases are polar and tend to bind hydrophilic compounds like glycopeptides. Typical mobile phases are aqueous buffers with organic modifiers such as acetonitrile. It is believed that the aqueous content of the liquid passing through the phase creates a water rich layer on the surface of the stationary phase. This allows for partitioning of solutes between the more organic mobile phase and the aqueous layer. Liquid with a higher proportion of organic solvent passing through the HILIC phase tends to cause polar compounds to partition into the aqueous layer on the HILIC medium. In contrast, liquid with a lower proportion of organic solvent passing through the HILIC phase tends to cause polar compounds to partition out of the aqueous layer and into the passing liquid.

b. HILIC Loads and Methods of Making

Provided herein, in some aspects, is a method for processing a proteolytic digest sample for use in a LC-MS analysis, the method comprising loading a HILIC load to a solid phase extraction column comprising a HILIC medium, wherein the HILIC load comprises: (a) an amount of the plurality of proteolytically digested peptides characterized by one or both of: (i) a ratio of the weight of the plurality of proteolytically digested peptides over the weight of the HILIC medium in the dry state of at least about 0.05, including at least about 0.06; and/or (ii) a ratio of the weight of the plurality of proteolytically digested peptides relative to the bed volume of the HILIC medium in the dry state of at least about 30 μg/μl, including at least about 40 μg/μl; and/or (b) a concentration of an organic solvent of at least about 70% (v/v). Also provided herein are methodologies for making the HILIC load having: (a) an amount of the plurality of proteolytically digested peptides characterized by one or both of: (i) a ratio of the weight of the plurality of proteolytically digested peptides over the weight of the HILIC medium in the dry state of at least about 0.05, including at least about 0.06; and/or (ii) a ratio of the weight of the plurality of proteolytically digested peptides relative to the bed volume of the HILIC medium in the dry state of at least about 30 μg/μl, including at least about 40 μg/μl; and/or (b) a concentration of an organic solvent of at least about 70% (v/v). For example, in some embodiments, the methods provided herein comprise: (a) obtaining a proteolytic digest sample; (b) removing a liquid content of the proteolytic digest sample; and (c) reconstituting the proteolytic digest sample to produce the HILIC load such that the HILIC load comprises: (A) an amount of the plurality of proteolytically digested peptides characterized by one or both of: (i) a ratio of the weight of the plurality of proteolytically digested peptides over the weight of the HILIC medium in the dry state of at least about 0.05, including at least about 0.06; and/or (ii) a ratio of the weight of the plurality of proteolytically digested peptides relative to the bed volume of the HILIC medium in the dry state of at least about 30 μg/μl, including at least about 40 μg/μl; and/or (b) a concentration of an organic solvent of at least about 70% (v/v).

In some embodiments, the HILIC load loaded to a solid phase extraction column has an amount of the plurality of proteolytically digested peptides characterized by a ratio of the weight of the plurality of proteolytically digested peptides over the weight of the HILIC medium in the dry state of about 0.05 to about 0.5, such as any of about 0.05 to about 0.35, about 0.05 to about 0.15, about 0.05 to about 0.25, or about 0.15 to about 0.25. In some embodiments, the HILIC load loaded to a solid phase extraction column has an amount of the plurality of proteolytically digested peptides characterized by a ratio of the weight of the plurality of proteolytically digested peptides over the weight of the HILIC medium in the dry state of at least about 0.05, such as at least about any of 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, or 0.35. In some embodiments, the HILIC load loaded to a solid phase extraction column has an amount of the plurality of proteolytically digested peptides characterized by a ratio of the weight of the plurality of proteolytically digested peptides over the weight of the HILIC medium in the dry state is about any of 0.05, 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, or 0.35. In some embodiments, the HILIC load loaded to a solid phase extraction column has an amount of the plurality of proteolytically digested peptides characterized by a ratio of the weight of the plurality of proteolytically digested peptides over the weight of the HILIC medium in the dry state of at least about 0.1, including about 0.1. In some embodiments, the HILIC load loaded to a solid phase extraction column has an amount of the plurality of proteolytically digested peptides characterized by a ratio of the weight of the plurality of proteolytically digested peptides over the weight of the HILIC medium in the dry state of at least about 0.2, including about 0.2. In any of the embodiments herein, the HILIC medium in the dry state has a weight of about 3 mg. In any of the embodiments herein, the HILIC medium in the dry state has a bed volume of about 5 μL. In some embodiments, the HILIC medium comprises silica particles covalently bonded with carbamoyl groups. In some embodiments, the solid phase extraction column comprises about 2.5 mg to about 3.5 mg, including about 3 mg, of a HILIC medium in a dry state, a bed volume of the HILIC medium in a dry state of about 4 μl to about 6 μl, including about 5 μl, and wherein the HILIC medium comprises silica particles covalently bonded with carbamoyl groups. In some embodiments, the solid phase extraction column is a GlykoPrep Cleanup cartridge (e.g., Product code: GS96-CU; WS0263).

In some embodiments, the HILIC load loaded to the solid phase extraction column has an amount of the plurality of proteolytically digested peptides characterized by a ratio of the weight of the plurality of proteolytically digested peptides relative to the bed volume of the HILIC medium in the dry state of about 30 μg/μl to about 200 μg/μl, such any of about 50 μg/μl to about 130 μg/μl, about 55 μg/μl to about 65 μg/μl, about 110 μg/μl to about 130 μg/μl, or about 115 μg/μl to about 125 μg/μl. In some embodiments, the HILIC load loaded to the solid phase extraction column has an amount of the plurality of proteolytically digested peptides characterized by a ratio of the weight of the plurality of proteolytically digested peptides relative to the bed volume of the HILIC medium in the dry state of at least about 30 μg/μl, such as at least about any of 35 μg/μl, 40 μg/μl, 45 μg/μl, 50 μg/μl, 55 μg/μl, 60 μg/μl, 65 μg/μl, 70 μg/μl, 75 μg/μl, 80 μg/μl, 85 μg/μl, 90 μg/μl, 95 μg/μl, 100 μg/μl, 105 μg/μl, 110 μg/μl, 115 μg/μl, 120 μg/μl, 125 μg/μl, 130 μg/μl, 135 μg/μl, 140 μg/μl, 145 μg/μl, 150 μg/μl, 155 μg/μl, 160 μg/μl, 165 μg/μl, 170 μg/μl, 175 μg/μl, 180 μg/μl, 185 μg/μl, 190 μg/μl, 195 μg/μl, or 200 μg/μl. In some embodiments, the HILIC load loaded to the solid phase extraction column has an amount of the plurality of proteolytically digested peptides characterized by a ratio of the weight of the plurality of proteolytically digested peptides relative to the bed volume of the HILIC medium in the dry state of about any of 30 μg/μl, 35 μg/μl, 40 μg/μl, 45 μg/μl, 50 μg/μl, 55 μg/μl, 60 μg/μl, 65 μg/μl, 70 μg/μl, 75 μg/μl, 80 μg/μl, 85 μg/μl, 90 μg/μl, 95 μg/μl, 100 μg/μl, 105 μg/μl, 110 μg/μl, 115 μg/μl, 120 μg/μl, 125 μg/μl, 130 μg/μl, 135 μg/μl, 140 μg/μl, 145 μg/μl, 150 μg/μl, 155 μg/μl, 160 μg/μl, 165 μg/μl, 170 μg/μl, 175 μg/μl, 180 μg/μl, 185 μg/μl, 190 μg/μl, 195 μg/μl, or 200 μg/μl. In some embodiments, the HILIC load loaded to the solid phase extraction column has an amount of the plurality of proteolytically digested peptides characterized by a ratio of the weight of the plurality of proteolytically digested peptides relative to the bed volume of the HILIC medium in the dry state of at least about 60 μg/μl, including about 60 μg/μl. In some embodiments, the HILIC load loaded to the solid phase extraction column has an amount of the plurality of proteolytically digested peptides characterized by a ratio of the weight of the plurality of proteolytically digested peptides relative to the bed volume of the HILIC medium in the dry state of at least about 120 μg/μl, including about 120 μg/μl. In any of the embodiments herein, the HILIC medium in the dry state has a weight of about 3 mg. In any of the embodiments herein, the HILIC medium in the dry state has a bed volume of about 5 μL. In some embodiments, the HILIC medium comprises silica particles covalently bonded with carbamoyl groups. In some embodiments, the solid phase extraction column comprises about 2.5 mg to about 3.5 mg, including about 3 mg, of a HILIC medium in a dry state, a bed volume of the HILIC medium in a dry state of about 4 μl to about 6 μl, including about 5 μl, and wherein the HILIC medium comprises silica particles covalently bonded with carbamoyl groups. In some embodiments, the solid phase extraction column is a GlykoPrep Cleanup cartridge (e.g., Product code: GS96-CU; WS0263).

In some embodiments, the HILIC load loaded to a solid phase extraction column has an amount of the plurality of proteolytically digested peptides characterized by a ratio of the weight of the plurality of proteolytically digested peptides over the weight of the HILIC medium in the dry state of about 0.05 to about 0.5, such as any of about 0.05 to about 0.35, about 0.05 to about 0.15, about 0.05 to about 0.25, or about 0.15 to about 0.25, and characterized by a ratio of the weight of the plurality of proteolytically digested peptides relative to the bed volume of the HILIC medium in the dry state of about 30 μg/μl to about 200 μg/μl, such any of about 50 μg/μl to about 130 μg/μl, about 55 μg/μl to about 65 μg/μl, about 110 μg/μl to about 130 μg/μl, or about 115 μg/μl to about 125 μg/μl. In some embodiments, the HILIC load loaded to a solid phase extraction column has an amount of the plurality of proteolytically digested peptides characterized by a ratio of the weight of the plurality of proteolytically digested peptides over the weight of the HILIC medium in the dry state of at least about 0.05, such as at least about any of 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, or 0.35, and characterized by a ratio of the weight of the plurality of proteolytically digested peptides relative to the bed volume of the HILIC medium in the dry state of at least about 30 μg/μl, such as at least about any of 35 μg/μl, 40 μg/μl, 45 μg/μl, 50 μg/μl, 55 μg/μl, 60 μg/μl, 65 μg/μl, 70 μg/μl, 75 μg/μl, 80 μg/μl, 85 μg/μl, 90 μg/μl, 95 μg/μl, 100 μg/μl, 105 μg/μl, 110 μg/μl, 115 μg/μl, 120 μg/μl, 125 μg/μl, 130 μg/μl, 135 μg/μl, 140 μg/μl, 145 μg/μl, 150 μg/μl, 155 μg/μl, 160 μg/μl, 165 μg/μl, 170 μg/μl, 175 μg/μl, 180 μg/μl, 185 μg/μl, 190 μg/μl, 195 μg/μl, or 200 μg/μl. In some embodiments, the HILIC load loaded to a solid phase extraction column has an amount of the plurality of proteolytically digested peptides characterized by a ratio of the weight of the plurality of proteolytically digested peptides over the weight of the HILIC medium in the dry state is about any of 0.05, 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, or 0.35, and characterized by a ratio of the weight of the plurality of proteolytically digested peptides relative to the bed volume of the HILIC medium in the dry state of about any of 30 μg/μl, 35 μg/μl, 40 μg/μl, 45 μg/μl, 50 μg/μl, 55 μg/μl, 60 μg/μl, 65 μg/μl, 70 μg/μl, 75 μg/μl, 80 μg/μl, 85 μg/μl, 90 μg/μl, 95 μg/μl, 100 μg/μl, 105 μg/μl, 110 μg/μl, 115 μg/μl, 120 μg/μl, 125 μg/μl, 130 μg/μl, 135 μg/μl, 140 μg/μl, 145 μg/μl, 150 μg/μl, 155 μg/μl, 160 μg/μl, 165 μg/μl, 170 μg/μl, 175 μg/μl, 180 μg/μl, 185 μg/μl, 190 μg/μl, 195 μg/μl, or 200 μg/μl. In some embodiments, the HILIC load loaded to a solid phase extraction column has an amount of the plurality of proteolytically digested peptides characterized by a ratio of the weight of the plurality of proteolytically digested peptides over the weight of the HILIC medium in the dry state of at least about 0.1, including about 0.1, and characterized by a ratio of the weight of the plurality of proteolytically digested peptides relative to the bed volume of the HILIC medium in the dry state of at least about 60 μg/μl, including about 60 μg/μl. In some embodiments, the HILIC load loaded to a solid phase extraction column has an amount of the plurality of proteolytically digested peptides characterized by a ratio of the weight of the plurality of proteolytically digested peptides over the weight of the HILIC medium in the dry state of at least about 0.2, including about 0.2, and characterized by a ratio of the weight of the plurality of proteolytically digested peptides relative to the bed volume of the HILIC medium in the dry state of at least about 120 μg/μl, including about 120 μg/μl. In any of the embodiments herein, the HILIC medium in the dry state has a weight of about 3 mg. In any of the embodiments herein, the HILIC medium in the dry state has a bed volume of about 5 μL. In some embodiments, the HILIC medium comprises silica particles covalently bonded with carbamoyl groups. In some embodiments, the solid phase extraction column comprises about 2.5 mg to about 3.5 mg, including about 3 mg, of a HILIC medium in a dry state, a bed volume of the HILIC medium in a dry state of about 4 μl to about 6 μl, including about 5 μl, and wherein the HILIC medium comprises silica particles covalently bonded with carbamoyl groups. In some embodiments, the solid phase extraction column is a GlykoPrep Cleanup cartridge (e.g., Product code: GS96-CU; WS0263).

In some embodiments, the HILIC load has an amount of a plurality of proteolytically digested peptides of about 150 μg to about 1,000 μg, such as any of about 200 μg to about 800 μg, about 200 μg to about 600 μg, about 250 μg to about 650 μg, or about 300 μg to about 600 μg. In some embodiments, the HILIC load has an amount of a plurality of proteolytically digested peptides of at least about 150 μg, such as at least about any of 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, or 1,000 μg. In some embodiments, the HILIC load has an amount of a plurality of proteolytically digested peptides of about 200 μg or more, such as about any of 250 μg or more, 300 μg or more, 350 μg or more, 400 μg or more, 450 μg or more, 500 μg or more, 550 μg or more, 600 μg or more, 650 μg or more, 700 μg or more, 750 μg or more, 800 μg or more, 850 μg or more, 900 μg or more, 950 μg or more, or 1,000 μg or more.

In some embodiments, the HILIC load has an amount of a plurality of proteolytically digested peptides of about 150 μg to about 1,000 μg, such as any of about 200 μg to about 800 μg, about 200 μg to about 600 μg, about 250 μg to about 650 μg, or about 300 μg to about 600 μg, wherein the solid phase extraction column comprises about 2.5 mg to about 3.5 mg, including about 3 mg, of a HILIC medium in a dry state, a bed volume of the HILIC medium in a dry state of about 4 μl to about 6 μl, including about 5 μl, and wherein the HILIC medium comprises silica particles covalently bonded with carbamoyl groups. In some embodiments, the HILIC load has an amount of a plurality of proteolytically digested peptides of at least about 150 μg, such as at least about any of 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, or 1,000 μg, wherein the solid phase extraction column comprises about 2.5 mg to about 3.5 mg, including about 3 mg, of a HILIC medium in a dry state, a bed volume of the HILIC medium in a dry state of about 4 μl to about 6 μl, including about 5 μl, and wherein the HILIC medium comprises silica particles covalently bonded with carbamoyl groups. In some embodiments, the HILIC load has an amount of a plurality of proteolytically digested peptides of about 200 μg or more, such as about any of 250 μg or more, 300 μg or more, 350 μg or more, 400 μg or more, 450 μg or more, 500 μg or more, 550 μg or more, 600 μg or more, 650 μg or more, 700 μg or more, 750 μg or more, 800 μg or more, 850 μg or more, 900 μg or more, 950 μg or more, or 1,000 μg or more, wherein the solid phase extraction column comprises about 2.5 mg to about 3.5 mg, including about 3 mg, of a HILIC medium in a dry state, a bed volume of the HILIC medium in a dry state of about 4 μl to about 6 μl, including about 5 μl, and wherein the HILIC medium comprises silica particles covalently bonded with carbamoyl groups. In some embodiments, the solid phase extraction column is a GlykoPrep Cleanup cartridge (e.g., Product code: GS96-CU; WS0263).

Polypeptide amounts described herein may be absolute or estimated amounts. In some embodiments, the amount of polypeptide content in any sample, or a derivative thereof, is a measured directly from said sample, or the derivative thereof, e.g., using a BCA quantification assay or a UV-VIS measurement at 280 nm. In some embodiments, the amount of polypeptide content in a sample, or a derivative thereof, is estimated based on a known, including reference standard, value for polypeptide content in the sample based on the origin of the sample, e.g., such as based on a known standard polypeptide concentration in human plasma or serum.

In some embodiments, provided is a method for processing a proteolytic digest sample for use in a LC-MS analysis, the method comprising loading a HILIC load to a solid phase extraction column comprising a HILIC medium, wherein the HILIC load comprises a concentration of an organic solvent of at least about 70% (v/v), such as at least about any of 71% (v/v), 72% (v/v), 73% (v/v), 74% (v/v), 75% (v/v), 76% (v/v), 77% (v/v), 78% (v/v), 79% (v/v), 80% (v/v), 81% (v/v), 82% (v/v), 83% (v/v), 84% (v/v), 85% (v/v), 86% (v/v), 87% (v/v), 88% (v/v), 89% (v/v), 90% (v/v), 91% (v/v), 92% (v/v), 93% (v/v), 94% (v/v), 95% (v/v), 96% (v/v), 97% (v/v), 98% (v/v), or 99% (v/v). In some embodiments, the HILIC load comprises a concentration of an organic solvent of about any of 70% (v/v), 71% (v/v), 72% (v/v), 73% (v/v), 74% (v/v), 75% (v/v), 76% (v/v), 77% (v/v), 78% (v/v), 79% (v/v), 80% (v/v), 81% (v/v), 82% (v/v), 83% (v/v), 84% (v/v), 85% (v/v), 86% (v/v), 87% (v/v), 88% (v/v), 89% (v/v), 90% (v/v), 91% (v/v), 92% (v/v), 93% (v/v), 94% (v/v), 95% (v/v), 96% (v/v), 97% (v/v), 98% (v/v), 99% (v/v), or 100% (v/v).

In some embodiments, the organic solvent comprises an aprotic solvent miscible in water. In some embodiments, the organic solvent is selected from the group consisting of acetonitrile, ethanol, methanol, tetrahydrofuran, and dioxane, or a combination thereof. In some embodiments, the organic solvent comprises acetonitrile (ACN). In some embodiments, the organic solvent is ACN.

In some embodiments, provided is a method for processing a proteolytic digest sample for use in a LC-MS analysis, the method comprising loading a HILIC load to a solid phase extraction column comprising a HILIC medium, wherein the HILIC load comprises a concentration of ACN of at least about 70% (v/v), such as at least about any of 71% (v/v), 72% (v/v), 73% (v/v), 74% (v/v), 75% (v/v), 76% (v/v), 77% (v/v), 78% (v/v), 79% (v/v), 80% (v/v), 81% (v/v), 82% (v/v), 83% (v/v), 84% (v/v), 85% (v/v), 86% (v/v), 87% (v/v), 88% (v/v), 89% (v/v), 90% (v/v), 91% (v/v), 92% (v/v), 93% (v/v), 94% (v/v), 95% (v/v), 96% (v/v), 97% (v/v), 98% (v/v), or 99% (v/v). In some embodiments, the HILIC load comprises a concentration of ACN of about any of 70% (v/v), 71% (v/v), 72% (v/v), 73% (v/v), 74% (v/v), 75% (v/v), 76% (v/v), 77% (v/v), 78% (v/v), 79% (v/v), 80% (v/v), 81% (v/v), 82% (v/v), 83% (v/v), 84% (v/v), 85% (v/v), 86% (v/v), 87% (v/v), 88% (v/v), 89% (v/v), 90% (v/v), 91% (v/v), 92% (v/v), 93% (v/v), 94% (v/v), 95% (v/v), 96% (v/v), 97% (v/v), 98% (v/v), 99% (v/v), or 100% (v/v). In some embodiments, the HILIC load comprises a concentration of ACN of about 80% (v/v).

In some aspects of the methods provided herein, the method comprises one or more steps for obtaining, such as making, a HILIC load. In some embodiments, the HILIC load comprises: (a) an amount of the plurality of proteolytically digested peptides characterized by one or both of: (i) a ratio of the weight of the plurality of proteolytically digested peptides over the weight of the HILIC medium in the dry state of at least about 0.05; and/or (ii) a ratio of the weight of the plurality of proteolytically digested peptides relative to the bed volume of the HILIC medium in the dry state of at least about 30 μg/μl, including such HILIC load in a set volume, e.g., about 220 μl or less; and/or (b) a concentration of an organic solvent of at least about 70% (v/v). In some embodiments, the proteolytic digest sample, such as resulting from a tryptic digestion, is of a different constitution than a HILIC load used in the methods described herein, such as HILIC load comprising (a) an amount of the plurality of proteolytically digested peptides characterized by one or both of: (i) a ratio of the weight of the plurality of proteolytically digested peptides over the weight of the HILIC medium in the dry state of at least about 0.05; and/or (ii) a ratio of the weight of the plurality of proteolytically digested peptides relative to the bed volume of the HILIC medium in the dry state of at least about 30 μg/μl; and/or (b) a concentration of an organic solvent of at least about 70% (v/v). In such embodiments, encompassed herein are method steps for processing a proteolytic digest sample to form a HILIC load. In some embodiments, the methods provided herein comprise: (a) obtaining a proteolytic digest sample; (b) removing a liquid content of the proteolytic digest sample; and (c) reconstituting the proteolytic digest sample to produce the HILIC load such that the HILIC load comprises: (A) an amount of the plurality of proteolytically digested peptides characterized by one or both of: (i) a ratio of the weight of the plurality of proteolytically digested peptides over the weight of the HILIC medium in the dry state of at least about 0.05; and/or (ii) a ratio of the weight of the plurality of proteolytically digested peptides relative to the bed volume of the HILIC medium in the dry state of at least about 30 μg/μl, including such HILIC load in a set volume, e.g., about 220 μl or less; and/or (B) a concentration of an organic solvent of at least about 70% (v/v).

In some embodiments, obtaining the HILIC load comprises reducing (such as removing) a liquid content from a proteolytic digest sample, such as resulting from a tryptic digestion, without substantial loss of a plurality of proteolytically digested peptides in the proteolytic digest sample. In some embodiments, the HILIC load comprises at least about 70%, 80%, or 90%, such as at least about any of 91%, 92%, 93%, 94%, 95%, 96%, 97%, 99%, or 100%, of a plurality of proteolytically digested peptides as compared to a proteolytic digest sample from which the HILIC load is obtained. In some embodiments, the reducing (such as removing) the liquid content from the proteolytic digested sample comprises performing a peptide concentrating technique with the proteolytically digested sample to obtain a precursor of the HILIC load such that (a) the precursor can be reconstituted with a reconstitution liquid comprising the organic solvent to obtain the HILIC load having a volume of 220 μL or less and a concentration of the organic solvent of at least about 70% (v/v); and (b) the resulting HILIC load comprises an amount of the plurality of proteolytically digested peptides of at least about 200 μg.

In some embodiments, the method comprises reducing a liquid content from the proteolytic digest sample to form a dried proteolytic digest sample; and reconstituting the dried proteolytic digest sample with a reconstitution liquid comprising the organic solvent to produce the HILIC load such that (a) the HILIC load has a volume of about 220 μL or less and a concentration of the organic solvent of at least about 70% (v/v); and (b) the HILIC load has an amount of the plurality of proteolytic peptides of at least about 200 μg.

In some embodiments, the reconstituting the dried proteolytic digest sample comprises: mixing the dried proteolytic digest sample with an amount of water to form a water mixture: sonicating the water mixture with a sonicator; mixing the water mixture with an amount of trifluoracetic acid (TFA) and acetonitrile (ACN), wherein the amount of TFA and ACN are such that the final concentration of TFA is about 1% (v/v) and the final concentration of ACN is about 80% (v/v); and sonicating the water mixture having the amount of TFA and ACN with a sonicator to produce the HILIC load. In some embodiments, the sonicating the water mixture with the sonicator comprises a water-based dissolution cycle, wherein the water-based dissolution cycle is repeated about 2 times to about 5 times, and wherein for each of the water-based dissolution cycles, the sonicating the water mixture is performed for about 5 minutes and a water reservoir of the sonicator is configured with ice to cool the water reservoir. In some embodiments, the sonicating the water mixture having the amount of TFA and ACN with the sonicator comprises an organic-based dissolution cycle, wherein the organic-based dissolution cycle is repeated about 2 times to about 3 times, and wherein for each of the organic-based dissolution cycles, the sonicating is performed for about 4 minutes and a water reservoir of the sonicator is configured with ice to cool the water reservoir.

In some embodiments, wherein the reducing (such as removing) the liquid content from the proteolytic digest sample comprises removing all or substantially all of the liquid content therefrom. In some embodiments, the peptide concentrating technique comprises a vacuum evaporation technique or a lyophilization technique. In some embodiments, the peptide concentrating technique comprises a SpeedVac.

In some embodiments, the proteolytic digest sample having reduced liquid content, such as a dried proteolytic digest sample, is reconstituted with one or more liquids. In some embodiments, the one or more liquids comprise one or more of water, TFA, ACN, or any combination thereof. In some embodiments, the dried proteolytic digest sample is reconstituted in stages, such as a first stage with water and a second stage with TFA and ACN, such that a HILIC load is obtained as described herein. In some embodiments, the volume of the HILIC load is about 10 μL to about 1 mL, such as about 100 to about 500 μL, about 50 μL to about 220 μL, or about 100 to about 220 μL. In some embodiments, the volume of the HILIC load is about 220 μL or less, such as about 210 μL or less, 200 μL or less, 190 μL or less, 180 μL or less, 170 μL or less, 160 μL or less, 150 μL or less, 140 μL or less, 130 μL or less, 120 μL or less, 110 μL or less, 100 μL or less, 90 μL or less, 80 μL or less, 70 μL or less, 60 μL or less, or 50 μL or less.

c. Additional Aspects of the Methods for Processing a Proteolytic Digest Sample

In certain aspects, the method provided herein comprises one or more additional steps involved with processing a proteolytic digest sample taught herein.

In some embodiments, the method comprises performing a washing step after loading a HILIC load to a solid phase extraction column comprising a HILIC medium and prior to subjecting the HILIC medium to n elution liquid, wherein the washing step comprises subjecting the HILIC medium to a wash liquid. In some embodiments, the wash liquid comprises an organic solvent at a concentration of at least about 90% (v/v), such as at least about 91% (v/v), 92% (v/v), 93% (v/v), 94% (v/v), 95% (v/v), 96% (v/v), 97% (v/v), 98% (v/v), or 99% (v/v). In some embodiments, the organic solvent is ACN. In some embodiments, the wash liquid further comprises TFA at a concentration of about 0.5% (v/v) to about 1% (v/v), including 1% (v/v).

In some embodiments, the elution liquid comprises a high percentage of an aqueous liquid, such as at least about 90% (v/v), such as at least about 91% (v/v), 92% (v/v), 93% (v/v), 94% (v/v), 95% (v/v), 96% (v/v), 97% (v/v), 98% (v/v), or 99% (v/v), of water. In some embodiments, the elution liquid further comprises about 0.05% (v/v) to about 0.5% (v/v) of TFA, such as about 0.1% TFA.

In some embodiments, the method comprises collecting the HILIC eluate, or a fraction thereof, from the solid phase extraction column, wherein the HILIC eluate comprises the at least one proteolytically digested glycopeptide. In some embodiments, after the collecting the HILIC eluate from the solid phase extraction column, the method further comprises reducing a liquid content of the collected HILIC eluate. In some embodiments, the method comprises subjecting the HILIC eluate to a peptide concentrating technique to produce a dried HILIC eluate, such as a peptide concentrating technique (e.g., SpeedVac).

In some embodiments, the method comprises reconstituting the dried HILIC eluate to form a sample suitable for introduction to the LC-MS system. In some embodiments, the dried HILIC eluate is reconstituted using 0.1% formic acid (FA) or TFA in water (e.g., in 30 μL of 0.1% FA in water).

As described in more detail herein, in some embodiments, the method comprises injecting the sample suitable for introduction to the LC-MS system into the LC-MS system. In some embodiments, the method comprises performing a mass spectrometry technique to obtain mass spectrometry data. In some embodiments, the method comprises identifying a peptide sequence of a glycopeptide from the mass spectrometry data. In some embodiments, the method comprises identifying a glycan attachment site of the glycopeptide from the mass spectrometry data. In some embodiments, the method comprises identifying a glycan structure of the glycopeptide from the mass spectrometry data. In some embodiments, the at least one glycopeptide comprises a glycan structure comprising one or more sialic acid moieties. In some embodiments, the proteolytic digest sample is obtained from a method for proteolytically digesting a fibrinogen-depleted sample, or a derivative thereof, comprising a glycoprotein.

In certain aspects, the method provided herein enable the enrichment of glycopeptide species derived from a fibrinogen-depleted sample, or a derivative thereof, such as via a proteolytic digest sample. In some embodiments, a glycopeptide concentration for a glycopeptide derived from the proteolytic digest sample is enriched by a factor of 30 or greater, such as any of 40 or greater, 50 or greater, 60 or greater, 70 or greater, 80 or greater, 90 or greater, 100 or greater, 110 or greater, 120 or greater, 130 or greater, 140 or greater, 150 or greater, 160 or greater, 170 or greater, 180 or greater, 190 or greater, or 200 or greater, with respect to a peptide concentration, wherein the peptide concentration represents an amount of a peptide that is associated with the same protein as the glycopeptide. In some embodiments, the method comprises: measuring a first plurality of peak area values for a first panel of glycopeptides; measuring a second plurality of peak area values for a second panel of unglycosylated peptides wherein each of the unglycosylated peptides of the second panel corresponds to each of the glycopeptides of the first panel by being attached to a same protein molecule before a proteolytic digestion; calculating a plurality of ratios by dividing each of the first plurality of peak area values with each of the second plurality of peak area values, respectively; and determining a median ratio from the plurality of ratios, wherein the median ratio is greater than 30.

In some embodiments, wherein the method for processing a proteolytic digest sample is performed in replicate (e.g., two or more aliquots of a proteolytic digest sample are processed using the same method for processing), the resulting coefficient of variation (CV) of a peak feature, such as peak area of the measured polypeptide, e.g., glycopolypeptide, is about 15% or less, such as about any of 14% or less, 13% or less, 12% or less, 11% or less, 10% or less, 9% or less, 8% or less, 7% or less, 6% or less, 5% or less, 4% or less, 3% or less, 2% or less, or 1% or less.

### F6. Methods for Performing a LC-MS Analysis of a Proteolytic Glycopeptide

In certain aspects, provided herein is a method for performing a LC-MS analysis on a sample comprising a proteolytic glycopeptide. In some embodiments, the liquid chromatography (LC) system is online with a mass spectrometer (i.e., proteolytic peptide species, including glycopeptides, are eluted from the LC system directing into the mass spectrometer via a mass spectrometer interface. In some embodiments, the LC technique comprises performing a chromatographic separation of one or more proteolytic peptides, including glycopeptides. In some embodiments, the one or more proteolytic peptides subjected to a chromatographic separation are obtained from a proteolytic digest sample, such as described herein.

In some embodiments, the method comprises introducing the proteolytically digested sample to a LC-MS system. In some embodiments, the method comprises performing a chromatographic separation of the proteolytically digested sample. In some embodiments, the chromatography separation comprises a period of diversion (i.e., diverted from the mass spectrometer interface, e.g., to a waste receptacle) of an initial eluate from the proteolytically digested sample. In some embodiments, the initial eluate (as assessed from the sample front) diverted from the mass spectrometer is about 1 column volume of the chromatographic column to about 5 column volumes of the chromatographic column, such as any of about 1 column volumes to about 4 column volumes, about 2 column volumes to about 5 column volumes, or about 3 column volumes to about 4 column volumes. In some embodiments, the initial eluate (as assessed from the sample front) diverted from the mass spectrometer is at least about 0.5 column volumes, such as at least about any of 1 column volume, 1.5 column volumes, 2 column volumes, 2.5 column volumes, 3 column volumes, 3.5 column volumes, 4 column volumes, 4.5 column volumes, or 5 column volumes. In some embodiments, the initial eluate (as assessed from the sample front) diverted from the mass spectrometer is about 5 column volumes or less, such as about any of 4.5 column volumes or less, 4 column volumes or less, 3.5 column volumes or less, 3 column volumes or less, 2.5 column volumes or less, 2 column volumes or less, 1.5 column volumes or less, 1 column volume or less, or 0.5 column volumes or less. In some embodiments, the initial eluate (as assessed from the sample front) diverted from the mass spectrometer is about any of 0.5 column volumes, 1 column volume, 1.5 column volumes, 2 column volumes, 2.5 column volumes, 3 column volumes, 3.5 column volumes, 4 column volumes, 4.5 column volumes, or 5 column volumes.

In some embodiments, the chromatographic separation comprises a gradient separation performing using mixtures of an aqueous mobile phase and an organic mobile phase. In some embodiments, the chromatographic separation comprises isocratic period, such as a period of at least about 90%, such as at least about any of 91%, 92%, 93%, 94%, 95%, 96%, 97%, 98%, 99%, or 100%, of an aqueous mobile phase to produce the initial eluate that is diverted from the mass spectrometer interface.

In some embodiments, the LC system comprises a reversed-phase chromatography column. In some embodiments, the reversed-phase column comprises an alkyl moiety, such as C18.

The present application contemplates a diverse array of additional features of LC-MS techniques for analyzing a sample comprising a glycopeptide using a mass spectrometer. In some embodiments, the liquid chromatography system comprises a high performance liquid chromatography system. In some embodiments, the liquid chromatography system comprises an ultra-high performance liquid chromatography system. In some embodiments, the liquid chromatography system comprises a high-flow liquid chromatography system. In some embodiments, the liquid chromatography system comprises a low-flow liquid chromatography system, such as a micro-flow liquid chromatography system or a nano-flow liquid chromatography system. In some embodiments, the liquid chromatography system is coupled, such as directly interfaced, with a mass spectrometer.

In some embodiment, the mass spectrometry technique comprises an ionization technique. Ionization techniques contemplated by the present application include techniques capable of charging polypeptides and peptide products, including glycopeptides. Thus, in some embodiments, the ionization technique is electrospray ionization (ESI). In some embodiments, the ionization technique is nano-electrospray ionization. In some embodiments, the ionization technique is atmospheric pressure chemical ionization. In some embodiments, the ionization technique is atmospheric pressure photoionization.

A diverse array of mass spectrometers are contemplated as compatible with the description, include high-resolution mass spectrometers and low-resolution mass spectrometers. In some embodiments, the mass spectrometer is a time-of-flight (TOF) mass spectrometer. In some embodiments, the mass spectrometer is a quadrupole time-of-flight (Q-TOF) mass spectrometer. In some embodiments, the mass spectrometer is a quadrupole ion trap time-of-flight (QIT-TOF) mass spectrometer. In some embodiments, the mass spectrometer is an ion trap. In some embodiments, the mass spectrometer is a single quadrupole. In some embodiments, the mass spectrometer is a triple quadrupole (QQQ). In some embodiments, the mass spectrometer is an orbitrap. In some embodiments, the mass spectrometer is a quadrupole orbitrap. In some embodiments, the mass spectrometer is a fourier transform ion cyclotron resonance (FT) mass spectrometer. In some embodiments, the mass spectrometer is a quadrupole fourier transform ion cyclotron resonance (Q-FT) mass spectrometer. In some embodiments, the mass spectrometry technique comprises positive ion mode. In some embodiments, the mass spectrometry technique comprises negative ion mode. In some embodiments, the mass spectrometry technique comprises an ion mobility mass spectrometry technique.

In some embodiments, the LC-MS technique comprises processing obtained signals MS from the mass spectrometer. In some embodiments, the LC-MS technique comprises peak detection. In some embodiments, the LC-MS technique comprises determining ionization intensity of an ionized peptide product. In some embodiments, the LC-MS technique comprises determining peak height of an ionized peptide product. In some embodiments, the LC-MS technique comprises determining peak area of an ionized peptide product. In some embodiments, the LC-MS technique comprises determining peak volume of an ionized peptide product. In some embodiments, the LC-MS technique comprises identifying an ionized peptide product by amino acid sequence. In some embodiments, the LC-MS technique comprises determining the site of a post-translational modification of an ionized peptide, such as the site of a glycosylation. In some embodiments, the LC-MS technique comprises determining the glycan structure, or a characteristic thereof, of an ionized peptide product. In some embodiments, the LC-MS technique comprises manually validating the ionized peptide product acid sequence assignments. In some embodiments, the LC-MS technique comprises a quantification technique.

### G6. Samples and Components Thereof

The methods provided herein are contemplated to be suitable for analyzing a diverse array of samples, such as blood-based biological samples. In some embodiments, the blood-derived sample is a plasma sample. In some embodiments, the blood-derived sample is a serum sample. Plasma is a fluid component of blood that is obtained when a clotting-prevention agent is added to whole blood and then the tube is centrifuged to separate the cellular material. The upper lighter colored liquid layer in the tube is removed as plasma. Common anti-coagulant agents ACD (anticoagulant citrate dextrose), Streck, EDTA (ethylenediaminetetraacetic acid), Heparin or Li-Heparin, oxalate fluoride, and a citrate phosphate dextrose adenine (CPDA). Serum is a fluid obtained when whole blood is allowed to clot in a tube and then centrifuged so that the clotted blood, including red cells, are at the bottom of the collection tube, leaving a straw-colored liquid above the clot. The straw-colored liquid in the tube is removed as serum. In some embodiments, the serum comprises fibrinogen.

The methods provided herein are particularly useful for the analysis of blood-derived samples comprising a glycoprotein, such as to generate glycopeptide containing specimens for analysis with a mass spectrometer. The methods provided herein, in some embodiments, enable the analysis of glycopeptides that elute during early or late phases of a reversed-phase chromatographic separation and are typically missed during conventional mass spectrometry approaches. For the situation where a sample contains hydrophilic salts and hydrophilic glycopeptides, it can be challenging to desalt the sample with a C18 solid phase extraction material without removing a significant portion of the hydrophilic glycopeptides that are needed for an analysis of the sample. For example, glycopeptides with an overall hydrophilic character may elute from a reversed-phase material, such as in a desalting column, and are washed away or not introduced to the mass spectrometer during a data acquisition phase of a mass spectrometry technique. In some embodiments, glycopeptides with an overall hydrophobic character may have a high affinity for a reversed-phase material, such as in a desalting column or a chromatography column, and are not properly eluted from a desalting column or during a data acquisition portion of a mass spectrometry technique.

In some embodiments, the method comprises an upstream sample preparation technique, such as for obtaining plasma or serum from a blood sample, performed prior to methods for proteolytically digesting a sample. In some embodiments, the upstream sample preparation technique comprises a cell lysis step. In some embodiments, the upstream sample preparation technique comprises a filtration step. In some embodiments, the upstream sample preparation technique comprises a dilution step. In some embodiments, the upstream sample preparation technique comprises a protein concentration determination step.

In some embodiments, the sample is obtained from an individual. In some embodiments, the sample is obtained from a human individual.

### H6. Systems, Kits, and Compositions

In certain aspects, contemplated herein are systems, kits, and compositions useful for performing the methods described herein. In some embodiments, provided herein is a system, kit, and/or composition useful for performing a proteolytic digestion of a biological sample comprising a glycoprotein as described herein. In some embodiments, provided herein is a system, kit, and/or composition useful for performing a LC-MS analysis of a proteolytic glycopeptide as described herein. In some embodiments, provided herein is a system, kit, and/or composition useful for performing a proteolytic digestion of a biological sample comprising a glycoprotein followed by LC-MS analysis of the proteolytic glycopeptide produced therefrom.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### Section 7—Methods and Systems for Analyzing Site-Specific Monomer Composition

### Peptide Structure Data Analysis

### I. Exemplary System for Peptide Structure Data Analysis

a. Analysis System for Peptide Structure Data Analysis

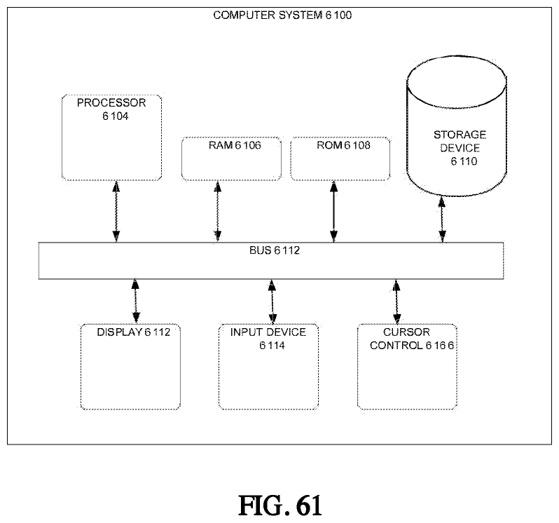

FIG. 60 is a block diagram of an analysis system 6000 in accordance with one or more embodiments. Analysis system 6000 can be used to both detect and analyze various peptide structures that have been associated to various disease states. Analysis system 6000 is one example of an implementation for a system that may be used to perform data analysis 108 in FIG. 1A. Thus, analysis system 6000 is described with continuing reference to workflow 100 as described in FIGS. 1A, 1B, and/or 1C.

Analysis system 6000 may include computing platform 6002 and data store 6004. In some embodiments, analysis system 6000 also includes display system 6006. Computing platform 6002 may take various forms. In one or more embodiments, computing platform 6002 includes a single computer (or computer system) or multiple computers in communication with each other. In other examples, computing platform 6002 takes the form of a cloud computing platform.

Data store 6004 and display system 6006 may each be in communication with computing platform 6002. In some examples, data store 6004, display system 6006, or both may be considered part of or otherwise integrated with computing platform 6002. Thus, in some examples, computing platform 6002, data store 6004, and display system 6006 may be separate components in communication with each other, but in other examples, some combination of these components may be integrated together. Communication between these different components may be implemented using any number of wired communications links, wireless communications links, optical communications links, or a combination thereof.

Analysis system 6000 includes, for example, peptide structure analyzer 6008, which may be implemented using hardware, software, firmware, or a combination thereof. In one or more embodiments, peptide structure analyzer 6008 is implemented using computing platform 6002.

Peptide structure analyzer 6008 receives peptide structure data 6010 for processing. Peptide structure data 6010 may be, for example, the peptide structure data that is output from sample preparation and processing 106 in FIGS. 1A, 1B, and 1C. Accordingly, peptide structure data 6010 may correspond to set of peptide structures 122 identified for biological sample 112 and may thereby correspond to biological sample 112.

Peptide structure data 6010 can be sent as input into peptide structure analyzer 6008, retrieved from data store 6004 or some other type of storage (e.g., cloud storage), accessed from cloud storage, or obtained in some other manner. In some cases, peptide structure data 6010 may be retrieved from data store 6004 in response to (e.g., directly or indirectly based on) receiving user input entered by a user via an input device.

Peptide structure analyzer 6008 includes model 6012 that is configured to receive peptide structure data 6010 for processing. Model 6012 may be implemented in any of a number of different ways. Model 6012 may be implemented using any number of models, functions, equations, algorithms, and/or other mathematical techniques.

In one or more embodiments, model 6012 includes machine learning system 6014, which may itself be comprised of any number of machine learning models and/or algorithms. For example, machine learning system 6014 may include, but is not limited to, at least one of a deep learning model, a neural network, a linear discriminant analysis model, a quadratic discriminant analysis model, a support vector machine, a random forest algorithm, a nearest neighbor algorithm (e.g., a k-Nearest Neighbors algorithm), a combined discriminant analysis model, a k-means clustering algorithm, an unsupervised model, a multivariable regression model, a penalized multivariable regression model, or another type of model. In various embodiments, model 6012 includes a machine learning system 6014 that comprises any number of or combination of the models or algorithms described above.

In various embodiments, model 6012 analyzes peptide structure data 6010 to generate disease indicator 6016 that indicates whether the biological sample is positive for a colorectal cancer disease state based on set of peptide structures 6018 identified as being associated with the colorectal cancer disease state. Peptide structure data 6010 may include quantification data for the plurality of peptide structures. Quantification data for a peptide structures can include at least one of an abundance, a relative abundance, a normalized abundance, a relative quantity, an adjusted quantity, a normalized quantity, a relative concentration, an adjusted concentration, or a normalized concentration. For example, peptide structure data 6010 may include a set of quantification metrics for each peptide structure of a plurality of peptide structures. A quantification metric for a peptide structure may be selected as one of a relative quantity, an adjusted quantity, a normalized quantity, a relative abundance, an adjusted abundance, and a normalized abundance. In some cases, a quantification metric for a peptide structure is selected from one of a relative concentration, an adjusted concentration, and a normalized concentration. In one or more embodiments, the quantification metrics used are normalized abundances. In this manner, peptide structure data 6010 may provide abundance information about the plurality of peptide structures with respect to biological sample 112.

Disease indicator 6016 may take various forms. In some examples, disease indicator 6016 includes a classification that indicates whether or not the subject is positive for the melanoma disease state. In various embodiments, disease indicator 6016 can include a score 6020. In some aspects, score 6020 indicates whether the melanoma disease state is present or not. For example, score 6020 may be, a probability score that indicates how likely it is that the biological sample 112 evidences the presence of the melanoma disease state. Alternatively or in addition, disease indicator 6016 includes a classification hat indicates whether or not the subject has immunotherapy responsive melanoma (i.e., will respond to immunotherapy treatment, for example immune checkpoint inhibitors such as ipilimumab, nivolumab, and/or pembrolizumab) or immunotherapy nonresponsive melanoma (i.e., will not respond to immunotherapy treatment, for example immune checkpoint inhibitors such as ipilimumab, nivolumab, and/or pembrolizumab). In such cases, score 6020 may be, for example, a probability score that indicates how likely it is that the biological sample 112 evidences the presence of an immunotherapy responsive melanoma.

In one or more embodiments, a peptide structure of set of peptide structures 6018 comprises a glycosylated peptide structure, or glycopeptide structure, that is defined by a peptide sequence and a glycan structure attached to a linking site of the peptide sequence quantity. For example, the peptide structure may be a glycopeptide or a portion of a glycopeptide. In some embodiments, a peptide structure of set of peptide structures 6018 comprises an aglycosylated peptide structure that is defined by a peptide sequence. For example, the peptide structure may be a peptide or a portion of a peptide and may be referred to as a quantification peptide.

Set of peptide structures 6018, and/or monomer weight scores derived therefrom, may be identified as being those most predictive or relevant to the melanoma disease state based on training of model 6012.

In various embodiments, machine learning system 6014 takes the form of binary classification model 6022. Binary classification model 6022 may include, for example, but is not limited to, a regression model. Binary classification model 6022 may include, for example, a penalized multivariable regression model that is trained to identify set of peptide structures 6018 from a plurality of (or panel of) peptide structures identified in various subjects. Binary classification model 6022 may be trained to identify weight coefficients for peptide structures and those peptide structures having non-zero weights or weight coefficients above a selected threshold (e.g., absolute weight coefficient above 0.0, 0.01, 0.05, 0.1, 0.015, 0.2, etc.) may be selected for inclusion in set of peptide structures 6018.

Peptide structure analyzer 6008 may generate final output 128 based on disease indicator 6016 output by model 6012. In other embodiments, final output 128 may be an output generated by model 6012.

In some embodiments, final output 128 includes disease indicator 6016. In one or more embodiments, final output 128 includes diagnosis output 6024, treatment output 6026, or both. Diagnosis output 6024 may include, for example, a diagnosis for the melanoma state. The diagnosis can include a positive diagnosis or a negative diagnosis for the melanoma disease state. Alternatively or in addition, the diagnosis can include a determination that the subject has immunotherapy responsive melanoma or immunotherapy nonresponsive melanoma. In some aspects, immunotherapy responsive melanoma describes melanoma responsive to treatment with immune checkpoint inhibitors. In some aspects, immunotherapy responsive melanoma describes melanoma responsive to combination treatment with ipilimumab and nivolumab. In some aspects, immunotherapy responsive melanoma describes melanoma responsive to combination treatment with pembrolizumab.

In one or more embodiments, when disease indicator 6016 and/or diagnosis output 6024 indicate a positive diagnosis for the melanoma disease state, a biopsy may be recommended. For example, a biopsy of the subject may be performed in response to disease indicator 6016 and/or diagnosis output 6024 indicating a positive diagnosis for the melanoma disease state. In some embodiments, peptide structure analyzer 6008 (or another system implemented on computing platform 6002) may generate a report recommending that a biopsy is to be performed for the subject in response to disease indicator 6016 and/or diagnosis output 6024 indicating a positive diagnosis for the melanoma disease state. In other embodiments, peptide structure analyzer 6008 may send diagnosis final output 128 to remote system 130 over one or more wireless, wired, and/or optical communications links and remote system 130 may generate a report recommending that a biopsy is to be performed for the subject in response to disease indicator 6016 and/or diagnosis output 6024 indicating a positive diagnosis for the adenoma or colorectal cancer disease state. The biopsy may be used to confirm the diagnosis to determine whether or not to administer treatment, which treatment to administer, and/or how quickly to administer treatment. When disease indicator 6016 and/or diagnosis output 6024 indicate a negative diagnosis for the melanoma disease state, the report that is generated by peptide structure analyzer 6008, remote system 130, or some other system implemented on computing platform 142 may recommend a period of monitoring for the subject. For example, a negative diagnosis indication by disease indicator 6016 and/or diagnosis output 6024 may thus help prevent unnecessary treatment or overtreatment of the subject.

Treatment output 6026 may include, for example, at least one of an identification of a treatment for the subject, a treatment plan for administering the treatment, or both. Treatment for melanoma may include, for example, but is not limited to, at least one of surgery, radiation therapy, a targeted drug therapy (e.g., one or more targeted therapeutic agents), chemotherapy (e.g., one or more chemotherapeutic agents), immunotherapy (e.g., one or more immunotherapeutic agents such as immune checkpoint inhibitors ipilimumab, nivolumab, and/or pembrolizumab), hormone therapy, neoadjuvant therapy, or some other form of treatment. The treatment plan may include, for example, but is not limited to, a timeline or schedule for administering the treatment, dosing information, other treatment-related information, or a combination thereof.

Final output 128 may be sent to remote system 130 for processing in some examples. In other embodiments, final output 128 may be displayed on graphical user interface 6030 in display system 6006 for viewing by a human operator.

b. Computer Implemented System

FIG. 61 is a block diagram of a computer system in accordance with various embodiments. Computer system 6100 may be an example of one implementation for computing platform 6002 described above in FIG. 60.

In one or more examples, computer system 6100 can include a bus 6102 or other communication mechanism for communicating information, and a processor 6104 coupled with bus 6102 for processing information. In various embodiments, computer system 6100 can also include a memory, which can be a random-access memory (RAM) 6106 or other dynamic storage device, coupled to bus 6102 for determining instructions to be executed by processor 6104. Memory also can be used for storing temporary variables or other intermediate information during execution of instructions to be executed by processor 6104. In various embodiments, computer system 6100 can further include a read only memory (ROM) 6108 or other static storage device coupled to bus 6102 for storing static information and instructions for processor 6104. A storage device 6110, such as a magnetic disk or optical disk, can be provided and coupled to bus 6102 for storing information and instructions.

In various embodiments, computer system 6100 can be coupled via bus 6102 to a display 6112, such as a cathode ray tube (CRT), liquid crystal display (LCD), or light emitting diode (LED) for displaying information to a computer user. An input device 6114, including alphanumeric and other keys, can be coupled to bus 6102 for communicating information and command selections to processor 6104. Another type of user input device is a cursor control 6116, such as a mouse, a joystick, a trackball, a gesture input device, a gaze-based input device, or cursor direction keys for communicating direction information and command selections to processor 6104 and for controlling cursor movement on display 6112. This input device 6114 typically has two degrees of freedom in two axes, a first axis (e.g., x) and a second axis (e.g., y), that allows the device to specify positions in a plane. However, it should be understood that input devices 6114 allowing for three-dimensional (e.g., x, y, and z) cursor movement are also contemplated herein.

Consistent with certain implementations of the present teachings, results can be provided by computer system 6100 in response to processor 6104 executing one or more sequences of one or more instructions contained in RAM 6106. Such instructions can be read into RAM 6106 from another computer-readable medium or computer-readable storage medium, such as storage device 6110. Execution of the sequences of instructions contained in RAM 6106 can cause processor 6104 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 6104 for execution. Such a medium can take many forms, including but not limited to, non-volatile media, volatile media, and transmission media. Examples of non-volatile media can include, but are not limited to, optical, solid state, magnetic disks, such as storage device 6110. Examples of volatile media can include, but are not limited to, dynamic memory, such as RAM 6106. Examples of transmission media can include, but are not limited to, coaxial cables, copper wire, and fiber optics, including the wires that comprise bus 6102.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 6104 of computer system 6100 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 6100 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 6100, whereby processor 6104 would execute the analyses and determinations provided by these engines, subject to instructions provided by any one of, or a combination of, the memory components RAM 6106, ROM, 6108, or storage device 6110 and user input provided via input device 6114.

### B7. Exemplary Methodologies Relating to Peptide Structure Data Analysis, Monomer Weight Score Calculations, and Diagnosis and Prognosis of Disease States

### I. Analyzing a Set of Peptide Structures

FIG. 36 is a flowchart of a process for analyzing a set of peptide structures in accordance with one or more embodiments. Process 3600 may be implemented using, for example, at least a portion of workflow 100 as described in FIG. 1A-1C, and/or analysis system 6000 as described in FIG. 60.

Step 3602 includes calculating a site occupancy score, for a given peptide structure at the linking site, as a function of an adjusted-raw abundance value for the given peptide structure and a sum of a set of adjusted-raw abundance values of the set of peptide structures. The adjusted-raw abundance values may be generated by normalizing a set of raw abundance values of the set of peptide structures to a corresponding reference run. The set of peptide structures can be a set of peptide structures comprising the same linking site and may be from a biological sample from a subject.

Step 3604 includes calculating a monomer weight score as a sum of the site occupancy score and a multiplier, where the multiplier is the number of a specific monomer in the set of peptide structures at the linking site. Step 3604 can additionally include calculating a peptide structure monomer weight score as a function of the site occupancy score and the number of a specific monomer for the given peptide structure. One or more peptide structure monomer weight scores may be used to calculate the monomer weight score.

Steps 3602 and 3604 can, in some cases, be repeated at least 1, 2, 3, 4, 5, 6, or more times, calculating site occupancy scores for a plurality of peptide structures and a plurality of monomer weight scores for a plurality of specific monomers at the linking site. In some cases, steps 3602 and 3604 are not repeated.

An additional step in process 3600 can include correlating the monomer weight score (or, in some cases, plurality of monomer weight scores) with an indication or disease state to determine a hazard ratio for the indication or disease state. An additional step in process 3600 can include generating a diagnosis output for the indication or disease state for the subject, using a predictive model, as a function of the monomer weight score, wherein the diagnosis output is one of a predictive probability or a risk score. An additional step in process 3600 can include generating a treatment output (e.g., an identification of a treatment to treat the subject such as identification of immunotherapy). An additional step in process 3600 can include generating a report identifying that a biological sample evidences the indication or disease state.

### II. Classifying a Biological Sample

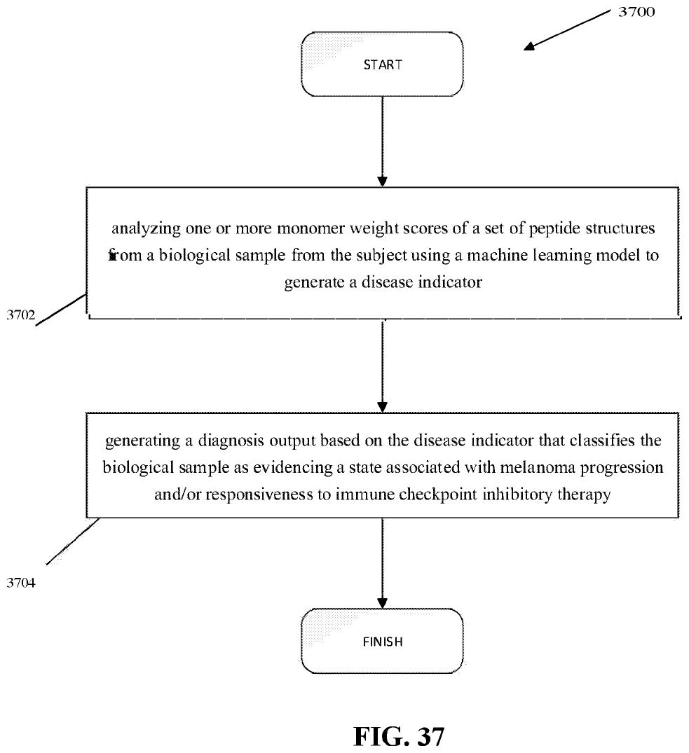

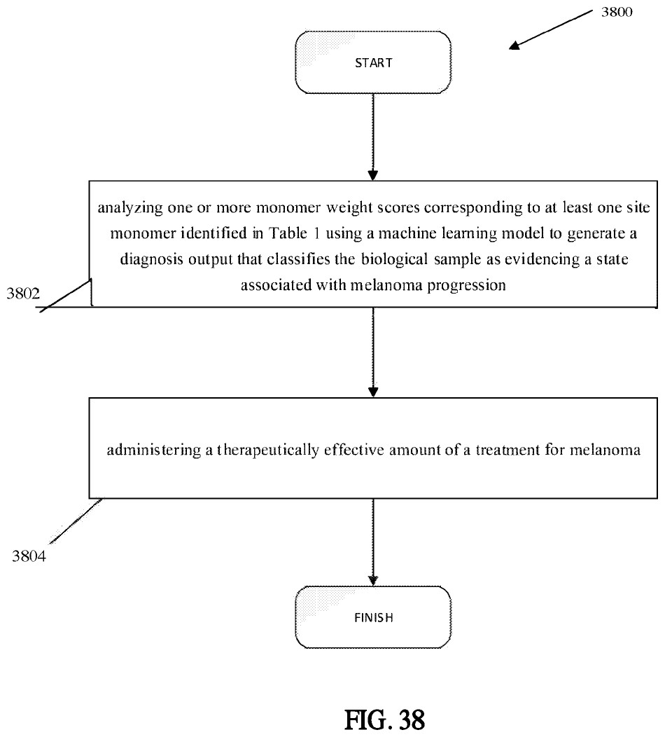

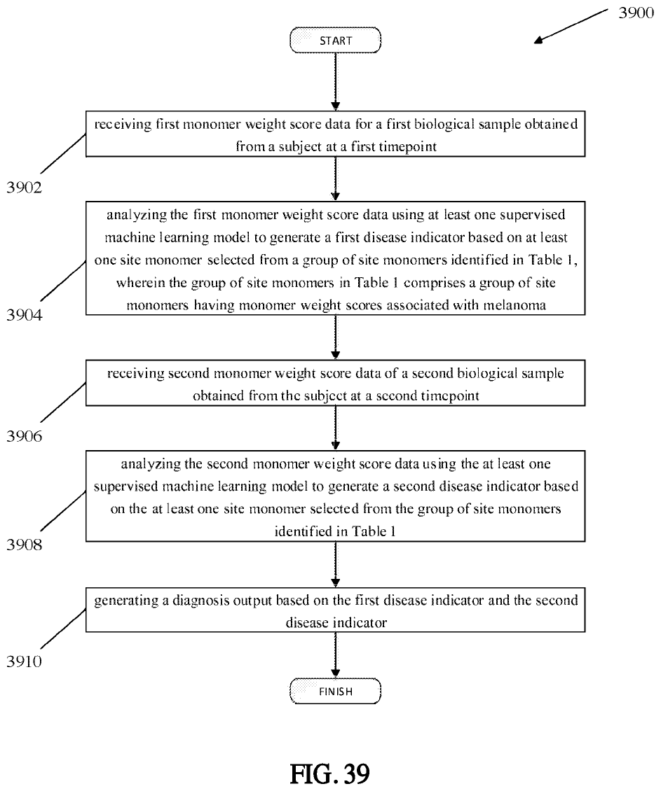

FIG. 37 is a flowchart of a process for classifying a biological sample in accordance with one or more embodiments. Process 3700 may be implemented using, for example, at least a portion of workflow 100 as described in FIGS. 1A-1C, and/or analysis system 6000 as described in FIG. 60.

Step 3702 includes analyzing one or more monomer weight scores of a set of peptide structures from a biological sample from a subject (e.g., a subject having or suspected of having a malignancy such as melanoma) using a machine learning model to generate a disease indicator. The set of peptide structures can be a set of peptide structures comprising the same linking site and may include glycosylated peptides and non-glycosylated peptides. The set of peptide structures may include peptides structures comprising one or more site monomers of Tables 16, 17, or 18, such that the one or more monomer weight scores are monomer weight scores of the one or more site monomers of Tables 16, 17, or 18. In step 3702, in accordance with various embodiments, the one or more monomer weight scores can be associated with the melanoma disease state.

In one or more embodiments, step 3702 may be implemented using a binary classification model (e.g., a regression model). In some examples, the regression model may be, for example, penalized multivariable regression model. In various embodiments, the disease indicator may be computed using a weight coefficient associated with each peptide structure, the weight coefficient of a corresponding peptide structure of the peptide structures may indicate the relative significance of the corresponding peptide structure to the disease indicator.

In some embodiments, step 3702 may include computing a peptide structure profile for the biological sample that identifies a weighted value for each peptide structure. The weighted value for a peptide structure of the peptide structures may be a product of a quantification metric for the peptide structure identified from the peptide structure data and a weight coefficient for the peptide structure. The disease indicator may be computed using the peptide structure profile. For example, the disease indicator may be a logit equal to the sum of the weighted values for the peptide structures plus an intercept value. The intercept value may be determined during the training of the model.

The peptide structure profile for a given peptide structure may include a corresponding feature—relative abundance, concentration, site occupancy, peptide structure monomer weight—for that peptide structure. The relative abundance may be a normalized relative abundance; the concentration may be normalized concentration. In some cases, two peptide structure profiles may be computed for the same peptide structure, each profile corresponding to a different feature. For example, a first peptide structure profile may include a relative abundance for a corresponding peptide structure and a second peptide structure profile may include a concentration for the same corresponding peptide structure.

In various embodiments, the disease indicator comprises a probability that the biological sample is positive for the melanoma disease state and the supervised machine learning model is configured to generate an output that identifies the biological sample as either evidencing (“positive for”) the melanoma disease state when the disease indicator is greater than a selected threshold or not evidencing (“negative for”) the melanoma disease state when the disease indicator is not greater than the selected threshold. The selected threshold may be, for example, 0.30, 0.35, 0.40, 0.45, 0.50, 0.55, 0.60, or some other threshold between 0.30 and 0.65. In one or more embodiments, the selected threshold is 0.5.

Step 3704 includes generating a diagnosis output based on the disease indicator that classifies the biological sample as evidencing a state associated with melanoma progression and/or responsiveness to immune checkpoint inhibitory therapy. In step 3704, in accordance with various embodiments, the diagnosis output can be, for example, diagnosis of a disease or malignancy (e.g., melanoma), identification of a treatment responsive malignancy (e.g., of immunotherapy responsive melanoma), and/or prognosis of disease severity.

The diagnosis output may include the disease indicator, or a diagnosis made based on the disease indicator. The diagnosis may be, for example, “positive” for the melanoma disease state if the biological sample evidences the melanoma disease state based on the disease indicator. The diagnosis may be, for example, “negative” if the biological sample does not evidence the melanoma disease state based on the disease indicator. A negative diagnosis may mean that the biological sample has a non-melanoma state. The negative diagnosis for the melanoma disease state can include at least one of a healthy state, or some other non-malignant state.

Generating the diagnosis output in step 3704 may include determining that the score falls above (or at or above) a selected threshold and generating a positive diagnosis for the melanoma disease state. Alternatively, step 3704 can include determining that the score falls below (or at or below) a selected threshold and generating a negative diagnosis for the adenoma or melanoma disease state. In some scoring systems, the score can include a probability score and the selected threshold can be 0.5. In other scoring systems, the selected threshold can fall within a range between 0.30 and 0.65.

In one or more embodiments, the final output in step 3704 may include a treatment output if the diagnosis output indicates a positive diagnosis for the melanoma disease state or a positive identification of immunotherapy responsive melanoma. The treatment output may include, for example, at least one of an identification of a treatment for the subject, a treatment plan for administering the treatment, or both. Treatment for melanoma may include, for example, but is not limited to, at least one of surgery, radiation therapy, a targeted drug therapy (e.g., one or more targeted therapeutic agents), chemotherapy (e.g., one or more chemotherapeutic agents), immunotherapy (e.g., one or more immunotherapeutic agents, e.g., immune checkpoint inhibitors such as ipilimumab, nivolumab, and/or pembrolizumab)), or some other form of treatment. The treatment plan may include, for example, but is not limited to, a timeline or schedule for administering the treatment, dosing information, other treatment-related information, or a combination thereof.

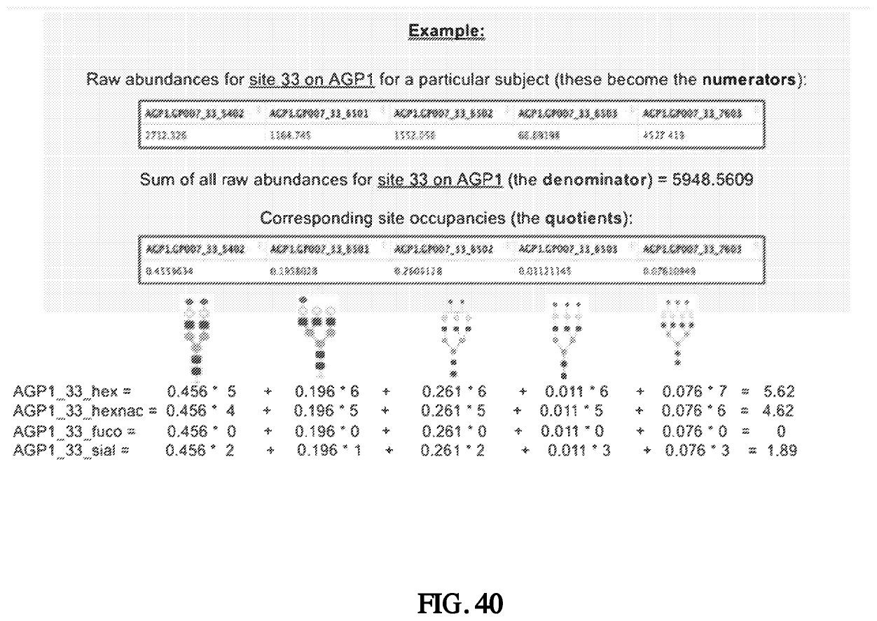

FIG. 38 is a flowchart of a process for treating a subject with melanoma. Step 3802 includes analyzing one or more monomer weight scores corresponding to at least one site monomer identified in Table 16 using a machine learning model to generate a diagnosis output that classifies the biological sample as evidencing a state associated with melanoma progression. Such analysis may be performed in line with the methods described above. Step 3804 includes administering a therapeutically effective amount of a treatment for melanoma. In some cases, for example where step 3802 identifies the subject as having immunotherapy sensitive melanoma, the treatment administered in step 3804 is an immunotherapy (e.g., immune checkpoint inhibitors such as ipilimumab, nivolumab, and/or pembrolizumab).

The group of site monomers in Tables 16, 17, or 18 include site monomers that have been determined relevant to distinguishing at least between melanoma and a healthy state and between immunotherapy responsive melanoma and immunotherapy nonresponsive melanoma. For example, the group of site monomers may be used to predict the probability of melanoma for use in clinically screening patients. In another example, the group of site monomers may be used to predict the probability of having immunotherapy responsive melanoma for use in informing treatment decisions.

In one or more embodiments, the at least 1 site monomer includes at least 1, at least 2, at least 3, at least 4, at least 5, or more of the site monomers listed in Table 16. In some embodiments, the at least 1 site monomer includes 1 or both of the site monomers listed in Table 18.

TABLE 16
Markers (Site Monomers) Associated with Melanoma

| | | | | | |
|---|---|---|---|---|---|
| | | | | Glycos | |
| | | | | site | |
| | | Protein | Pept | within | |
| Marker (Site | Protein | SEQ | SEQ | Prot | |
| Monomer) | Name | ID NO | ID NO | SEQ | Monomer |

| | | | | |
|---|---|---|---|---|
| TRFE_630_fuco | 152 | 1364 | 360 | Fucose |
| CFAH_882_fuco | 253 | 1465 | 882 | Fucose |
| HPT_184_fuco | 354 | 1566 | 184 | Fucose |
| FETUA_156_fuco | 455 | 1667 | 156 | Fucose |
| IC1_253_fuco | 556 | 1768 | 253 | Fucose |
| HPT_207_fuco | 354 | 1566 | 207 | Fucose |
| A1AT_70_fuco | 657 | 1869 | 70 | Fucose |
| HEMO_187_fuco | 758 | 1970 | 187 | Fucose |
| AGP12_56_fuco | 859 | 2071 | 56 | Fucose |
| CERU_762_fuco | 960 | 2172 | 762 | Fucose |
| HPT_241_fuco | 354 | 1566 | 241 | Fucose |
| VTNC_242_fuco | 1061 | 2273 | 242 | Fucose |
| IC1_48_sial | 556 | 1768 | 48 | Sialic Acid |
| CLUS_86_sial | 1162 | 2374 | 86 | Sialic Acid |
| APOE_308_sial | 1263 | 2475 | 308 | Sialic Acid |
| HPT_241_sial | 354 | 1566 | 241 | Sialic Acid |

TABLE 17
Fucose Markers Associated with Melanoma

| | | | | Glycos | |
|---|---|---|---|---|---|
| | | | | site | |
| | | Protein | Pept | within | |
| Marker (Site | Protein | SEQ | SEQ | Prot | |
| Monomer) | Name | ID NO | ID NO | SEQ | Monomer |
| TRFE_630_fuco | Serotransferrin | 152 | 1364 | 360 | Fucose |
| CFAH_882_fuco | Complement factor H | 253 | 1465 | 882 | Fucose |
| HPT_184_fuco | Haptoglobin | 354 | 1566 | 184 | Fucose |
| FETUA_156_fuco | Alpha-2-HS- | 455 | 1667 | 156 | Fucose |
| | glycoprotein | | | | |
| IC1_253_fuco | Plasma protease C1 | 556 | 1768 | 253 | Fucose |
| | inhibitor | | | | |
| HPT_207 fuco | Haptoglobin | 354 | 1566 | 207 | Fucose |
| A1AT_70_fuco | Alpha-1-antitrypsin | 657 | 1869 | 70 | Fucose |
| HEMO_187_fuco | Hemopexin | 758 | 1970 | 187 | Fucose |
| AGP12_56_fuco | Alpha-1-acid | 859 | 2071 | 56 | Fucose |
| | glycoprotein 1, | | | | |
| | Alpha-1-acid | | | | |
| | glycoprotein 2 | | | | |
| CERU_762_fuco | Ceruloplasmin | 960 | 2172 | 762 | Fucose |
| HPT_241_fuco | Haptoglobin | 354 | 1566 | 241 | Fucose |
| VTNC_242_fuco | Vitronectin | 1061 | 2273 | 242 | Fucose |

TABLE 18
Fucose Markers Strongly Associated with Melanoma

| | | | | Glycos | |
|---|---|---|---|---|---|
| | | | | site | |
| | | Protein | Pept | within | |
| Marker (Site | Protein | SEQ | SEQ | Prot | |
| Monomer) | Name | ID NO | ID NO | SEQ | Monomer |
| CFAH_882_fuco | Complement | 253 | 1465 | 882 | Fucose |
| | factor H | | | | |
| HPT_184_fuco | Hap toglobin | 354 | 1566 | 184 | Fucose |

### III. Monitoring a Subject for a Melanoma Disease State

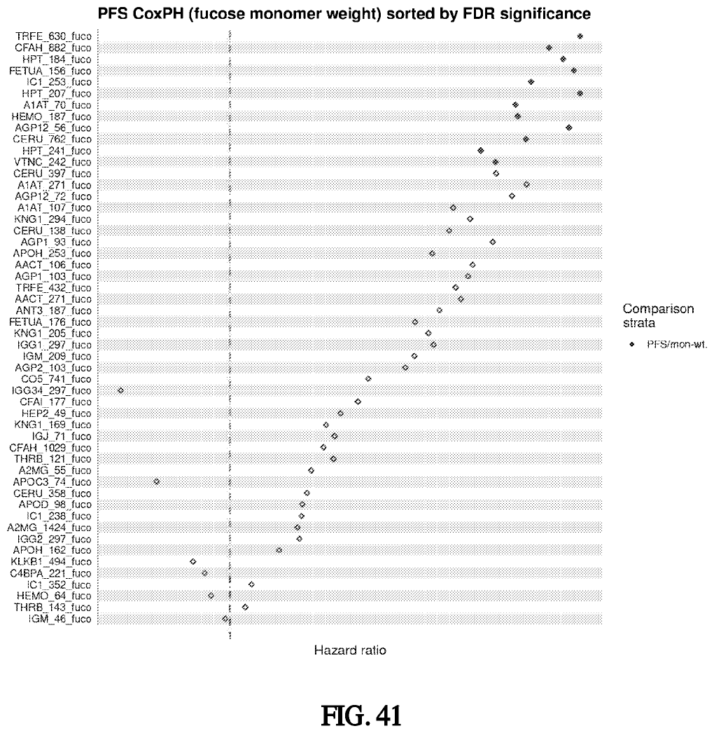

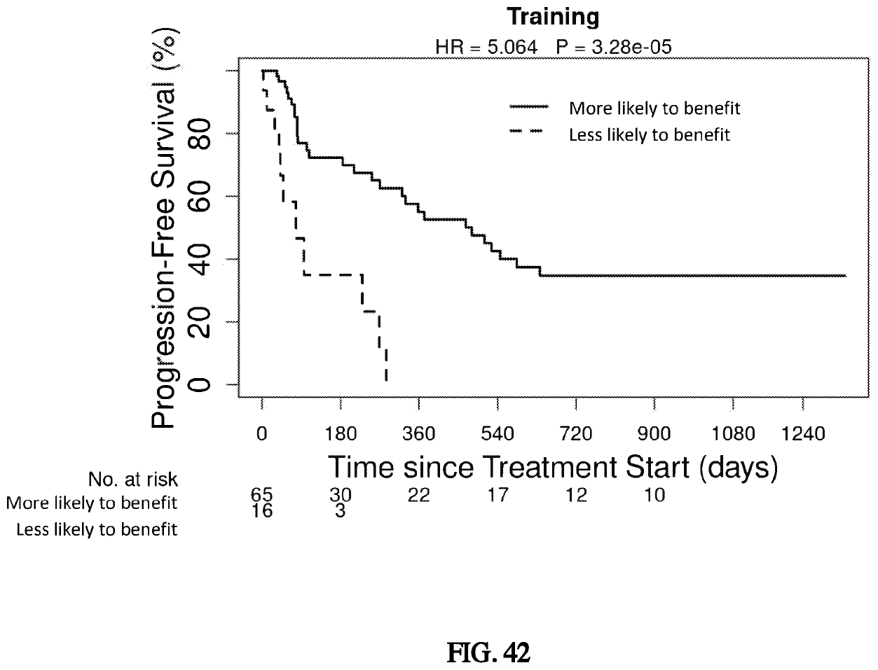

FIG. 39 is a flowchart of a process for monitoring a subject for a melanoma disease state in accordance with one or more embodiments. Process 3900 may be implemented using, for example, at least a portion of workflow 100 as described in FIGS. 1A-1C, and/or analysis system 6000 as described in FIG. 60.

Step 3902 includes receiving first monomer weight score data for a first biological sample obtained from a subject at a first timepoint.

Step 3904 includes analyzing the first monomer weight score data using a supervised machine learning model to generate a first disease indicator based on at least 1 site monomer from a group of site monomers identified in Table 16, 17, or 18. The group of site monomers in Tables 16, 17, or 18 includes a group of site monomers associated with a melanoma disease state in accordance with various embodiments. The supervised machine can be a binary classification model. In some embodiments, the binary classification model can be a logistical regression model.

Step 3906 includes receiving second monomer weight score data of a second biological sample obtained from the subject at a second timepoint.

Step 3908 includes analyzing the second monomer weight score data using the supervised machine learning model to generate a second disease indicator based on the at least 1 site monomer selected from the group of site monomers identified in Tables 16, 17, or 18.

Step 3910 includes generating a diagnosis output based on the first disease indicator and the second disease indicator. Generating the diagnostic output can include comparing the second disease indicator to the first disease indicator.

In some embodiments, the first disease indicator indicates that the first biological sample evidences the negative diagnosis for the melanoma disease state and the second biological sample evidences the positive diagnosis for the melanoma disease. In other embodiments, the diagnosis output identifies whether a non-melanoma disease state has progressed to the melanoma disease state wherein the non-melanoma disease state includes either a healthy state, or a control state. in some embodiments, the first disease indicator indicates that the first biological sample evidences immunotherapy sensitive melanoma and the second biological sample evidences immunotherapy resistant melanoma.

### C7. Peptide Structure and Product Compositions, Kits and Reagents

Table 19 identifies the proteins corresponding to the site monomers or peptide structures of Tables 16, 17, 18, 29, and 23A-D. Table 19 identifies a corresponding protein abbreviation and protein name for each of protein SEQ ID NOS: 52-63, 76, and 87-94. Further, Table 19 identifies a corresponding Uniprot ID for each of protein SEQ ID NOS: 52-63, 76, and 87-94.

TABLE 19
Protein SEQ ID NOS

| Protein | Protein | Uniprot | Prot Seq |
|---|---|---|---|
| Abbrev. | Name | ID | ID NO. |
| TRFE | Serotransferrin | P02787 | 152 |
| CFAH | Complement factor H | P08603 | 253 |
| HPT | Haptoglobin | P00738 | 354 |
| FETUA | Alpha-2-HS- | P02765 | 455 |
| | glycoprotein | | |
| IC1 | Plasma protease C1 | P05155 | 556 |
| | inhibitor | | |
| A1AT | Alpha-1-antitrypsin | P01009 | 657 |
| HEMO | Hemopexin | P02790 | 758 |
| AGP12 | Alpha-1-acid | P02763, | 859 |
| | glycoprotein 1, | P19652 | |
| | Alpha-1-acid | | |
| | glycoprotein 2 | | |
| CERU | Ceruloplasmin | P00450 | 960 |
| VTNC | Vitronectin | P04004 | 1061 |
| CLUS | Clusterin | P10909 | 1162 |
| APOE | Apolipoprotein E | P02649 | 1263 |
| APOH | Beta-2- | P02749 | 2576 |
| | glycoprotein1 | | |
| AACT | Alpha-1- | P01011 | 3787 |
| | antichymotrypsin | | |
| APOB | Apolipoprotein B-100 | P04114 | 3888 |
| APOM | Apolipoprotein M | O95445 | 3989 |
| IGG1 | Immunoglobulin | P01857 | 4090 |
| | heavy constant | | |
| | gamma 1 | | |
| IGG2 | Immunoglobulin | P01859 | 4191 |
| | heavy constant | | |
| | gamma 2 | | |
| KLKB1 | Plasma Kallikrein | P03952 | 4292 |
| KLKB1 | Plasma Kallikrein | P03952 | 4393 |
| FINC | Fibronectin | P02751 | 494 |

Aspects of the disclosure include kits comprising one or more compositions, each comprising one or more peptide structures of the disclosure that can be used as assay standards, and instructions for use. Kits in accordance with one or more embodiments described herein may include a label indicating the intended use of the contents of the kit. The term “label” as used herein with respect to a kit includes any writing, or recorded material supplied on or with a kit, or that otherwise accompanies a kit.

The peptide structures and the transitions produced therefrom, as well as monomer weight scores calculated therefrom as described herein, may be useful for diagnosing and treating a melanoma disease state. A transition includes a precursor ion and at least one product ion grouping. As reviewed herein, the peptide structures, as well as their corresponding precursor ion and product ion groupings (these ions having defined m/z ratios or m/z ratios that fall within the m/z ranges identified herein), can be used in mass spectrometry-based analyses to diagnose and facilitate treatment of diseases, such as, for example, melanoma.

Aspects of the disclosure include methods for analyzing one or more peptide structures, as described herein. In some embodiments, the methods involve processing a sample from a patient to generate a prepared sample that can be inputted into a mass spectrometry system (e.g., a reaction monitoring mass spectrometry system). In certain embodiments, processing the sample can comprise performing one or more of: a denaturation procedure, a reduction procedure, an alkylation procedure, and a digestion procedure. The denaturation and reduction procedures may be implemented in a manner similar to, for example, denaturation and reduction 202 in FIGS. 1B-1C. The alkylation procedure may be implemented in a manner similar to, for example, alkylation procedure 204 in FIGS. 1B-1C. The digestion procedure may be implemented in a manner similar to, for example, digestion procedure 206 in FIGS. 1B-1C.

In some embodiments, the methods for analyzing one or more peptide structures involve detecting a set of product ions generated by a reaction monitoring mass spectrometry system in which one or more product ions may correspond to each of the one or more peptide structures that have been inputted into the mass spectrometry system. As described herein, each peptide structure can be converted into a set of product ions having a defined m/z ratio. In some embodiments, the methods involve generating quantification (e.g., abundance) data for the one or more product ions detected using the reaction monitoring mass spectrometry system.

In some embodiments, the methods further comprise generating a diagnosis output using the quantification data and a model that has been trained using supervised or unsupervised machine learning. In certain embodiments, the reaction monitoring mass spectrometry system may include multiple/selected reaction monitoring mass spectrometry (MRM/SRM-MS) to detect the one or more product ions and generate the quantification data.

### I. Representative Experimental Results

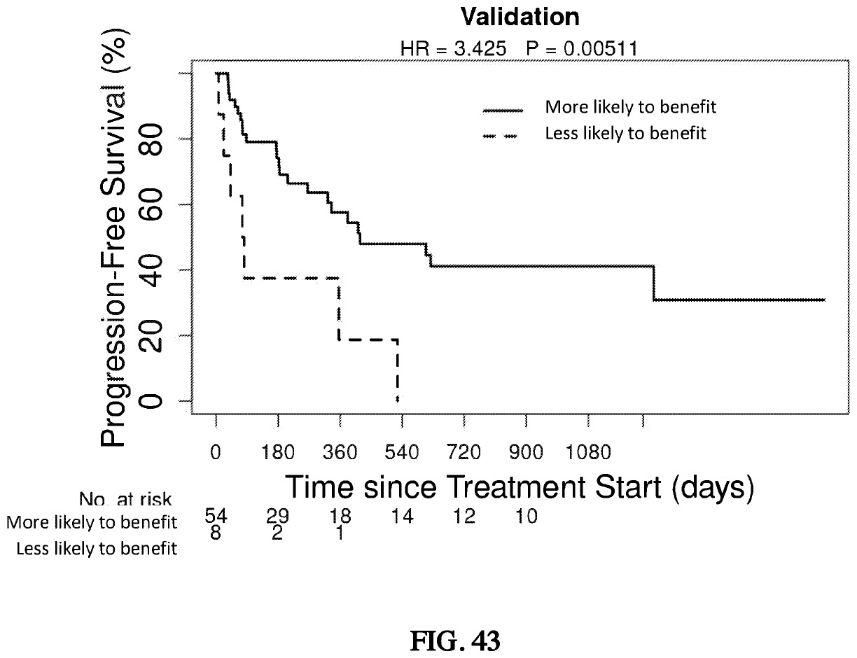

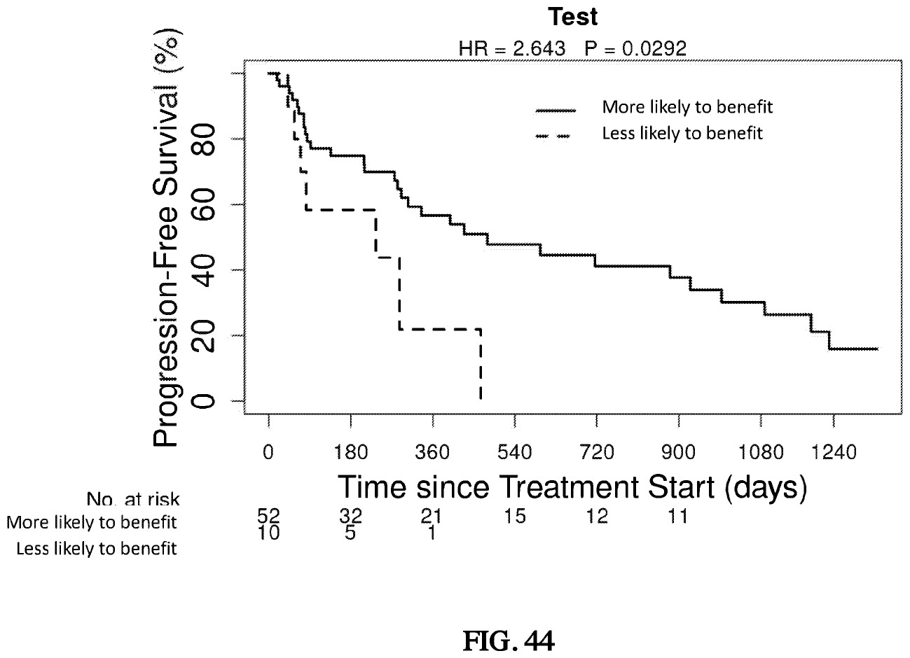

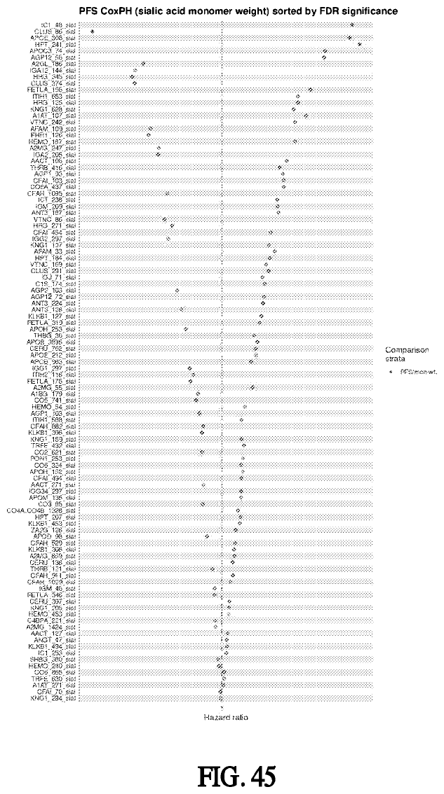

To assess the association of individual peptide structures (biomarkers) with metastatic advanced melanoma, univariate age- and sex-adjusted Cox regression analysis was performed on a cohort of 205 samples sourced from Massachusetts General Hospital. Results of the analysis are summarized below with reference to Tables 20-21 and FIGS. 41-45.

TABLE 20
all fucose markers

| Marker (Site Monomer) | HR | P | FDR |
|---|---|---|---|
| TRFE_630_fuco | 1.544 | 3.66E−05 | 6.18E−03 |
| CFAH_882_fuco | 1.486 | 5.62E−05 | 6.18E−03 |
| HPT_184_fuco | 1.512 | 6.04E−05 | 6.18E−03 |
| FETUA_156_fuco | 1.532 | 2.26E−04 | 1.59E−02 |
| IC1_253_fuco | 1.453 | 2.60E−04 | 1.59E−02 |
| HPT_207_fuco | 1.544 | 4.23E−04 | 1.86E−02 |
| A1AT_70_fuco | 1.425 | 5.91E−04 | 1.86E−02 |
| HEMO_187_fuco | 1.429 | 6.76E−04 | 1.89E−02 |
| AGP12_56_fuco | 1.523 | 7.54E−04 | 1.93E−02 |
| CERU_762_fuco | 1.444 | 1.08E−03 | 2.38E−02 |
| HPT_241_fuco | 1.365 | 1.50E−03 | 2.38E−02 |
| VTNC_242_fuco | 1.39 | 3.55E−03 | 4.36E−02 |

TABLE 21
2 fucose markers

| Marker (Site Monomer) | Coefficient |
|---|---|
| CFAH_882_fuco | 0.2498674 |
| HPT_184_fuco | 0.2396704 |

### D7. Exemplary Embodiments for Melanoma Diagnosis, Prognosis, and Treatment

The present disclosure concerns embodiments for systems, methods, and compositions related to identification of melanoma, risk thereof, or responsiveness thereof to a particular treatment (e.g., immunotherapy) in an individual. The embodiments concern classifying biological samples, measuring for one or more certain markers from a biological sample, assaying for one or more certain markers from a biological sample, determining the presence of one or more certain markers from a biological sample, and so forth. Embodiments of the disclosure utilize models that accurately either identify, for example, that an individual has melanoma, has a higher risk for melanoma over the general population, has melanoma that is responsive to immunotherapy, or has melanoma that is not responsive to immunotherapy, based on the presence of one or more markers in sample(s) from the individual. The individual may or may not be at a higher risk for melanoma based on one or more risk factors. An individual may be at risk for melanoma based on family or personal history; age; having one or more genetic markers associated with melanoma; and/or other risk factor for melanoma recognized in the art.

In various embodiments of the disclosure, an individual is in need of identifying whether or not they have melanoma, or a risk thereof. The individual may be subjected to measuring or testing for one or more markers encompassed herein as a matter of routine health maintenance or because of a specific concern, for example, such as the presence of one or more risk factors and/or one or more symptoms of melanoma. The individual may be in need of such identification based on any one of the risk factors noted above, or the individual may be in need of such identification based on having one or more symptoms of melanoma.

In some cases, the analysis of the sample of the individual as described herein is the sole test utilized for identifying melanoma, whereas in other cases a medical provider may utilize one or more other tests, such as biopsy, ultrasound; magnetic resonance imaging; CT scan; and so forth. In particular embodiments, calculating a monomer weight score for one or more site monomers markers of Table 16, 17, or 18 is utilized alone or in conjunction with one or more of these tests.

In some embodiments, the systems, methods, and compositions encompassed herein are sufficiently specific to utilize markers that distinguish between control and melanoma. In some embodiments, the systems, methods, and compositions encompassed herein are sufficiently specific to utilize markers that distinguish between melanoma that is responsive to immunotherapy and melanoma that is not responsive to immunotherapy. In some embodiments, the markers are accurate regardless of the status of one or more characteristics of the individual: biological sex, sample source, sample collection, or age, as examples.

In some embodiments, the individual is suspected of having melanoma or is at risk for melanoma and is in need of diagnosis thereof in addition to identification whether it is a particular stage of melanoma. In various embodiments, the individual is known to have melanoma and is in need of determining whether it is early stage melanoma or late stage melanoma, such as to determine a treatment regimen for the cancer. In specific embodiments, the same test that identifies whether an individual has melanoma determines whether the melanoma is early stage or late stage or a particular stage.

In various embodiments, the sample for analysis for melanoma identification may be a solid or fluid from the individual, such as skin, peripheral blood, serum, and/or plasma from the individual. The present disclosure provides for measuring for one or more circulating glycoproteins, glycopeptides, or non-glycosylated peptides in blood, serum, or plasma to diagnose or identify the presence of melanoma and/or to identify early stage or late stage melanoma in an individual. In various embodiments, the sample is measured for monomer weight scores for 1, 2, 3, 4, 5, or more of the site monomers of Table 16, 17, or 18.

Embodiments of the disclosure include methods of classifying samples, including skin, peripheral blood, serum, or plasma samples, from an individual suspected of having, known to have, or at risk for having melanoma by measuring from the sample for one or more glycopeptides and/or non-glycosylated peptides encompassed herein. The methods encompass whether or not melanoma is identified in the individual. In some cases, the measuring identifies the individual as not having melanoma or as having melanoma. In some cases, the measuring identifies the individual as having melanoma that is responsive to immunotherapy or as having melanoma that is not responsive to immunotherapy. The measuring may identify the individual as having a particular stage of melanoma, including at least early stage or late stage. In specific cases, the measuring comprises successive or concomitant steps of identifying that the individual has melanoma and whether the individual has early stage or late stage melanoma.

In various embodiments, an individual at risk for having melanoma is subjected to methods of the disclosure to identify, or not, the presence of melanoma. Such methods also measure for one or more glycopeptides and/or non-glycosylated peptides encompassed herein, as well as calculate monomer weight scores for one or more site monomers encompassed herein. The individual may be of any kind, although in specific cases individual at risk for having melanoma has a family history or one or more other risk factors.

Embodiments of the disclosure include methods of predicting that an individual will have melanoma, including early stage or late stage melanoma, or identifying early stage or late stage melanoma in an individual, or predicting that an individual has a melanoma that is responsive to immunotherapy by determining a monomer weight score for one or more site monomers from Table 16 in one or more samples from the individual. The individual may be known to have melanoma or may be suspected of having melanoma In various embodiments, the sample is measured for monomer weight scores for 1, 2, or more of the site monomers of Table 16, 17, or 18.

In embodiments wherein the measuring identifies the individual as having melanoma, the individual may be recommended to take action to treat the melanoma, such as with at least one of radiation therapy, chemotherapy or drug therapy (Bevacizumab, evacizumab, Irinotecan Hydrochloride, Capecitabine, Cetuximab, Ramucirumab, Oxaliplatin, Cetuximab, 5-FU, Ipilimumab, Irinotecan Hydrochloride, Pembrolizumab, Leucovorin Calcium, Trifluridine and Tipiracil Hydrochloride, Nivolumab, Nivolumab, Oxaliplatin. Panitumumab, Pembrolizumab, Ramucirumab, Regorafenib, Regorafenib, Panitumumab, Ziv-Aflibercept), immunotherapy (e.g., immune checkpoint inhibitors such as ipilimumab, nivolumab, ipilimumab plus nivolumab, and/or pembrolizumab), chemoradiotherapy, surgery, hormone therapy and/or a targeted drug therapy, as examples.

### E7. Cancer Therapy

In some aspects, the disclosed methods comprise administering a cancer therapy to a subject or patient. The cancer therapy may be chosen based on an analysis or method described herein. In some aspects, the cancer therapy comprises a local cancer therapy. In some aspects, the cancer therapy excludes a systemic cancer therapy. In some aspects, the cancer therapy excludes a local therapy. In some aspects, the cancer therapy comprises a local cancer therapy without the administration of a systemic cancer therapy. In some aspects, the cancer therapy comprises an immunotherapy, which may be an immune blockade or immune checkpoint inhibitor therapy. In some aspects, the cancer therapy comprises radiotherapy. Any of these cancer therapies may also be excluded. Combinations of these therapies may also be administered.

The term “cancer,” as used herein, may be used to describe a solid tumor, metastatic cancer, or non-metastatic cancer. In certain aspects, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

### I. Radiotherapy

In some aspects, the disclosed methods comprise administering radiotherapy, such as ionizing radiation, as a cancer therapy to a subject or patient. As used herein, “ionizing radiation” means radiation comprising particles or photons that have sufficient energy or can produce sufficient energy via nuclear interactions to produce ionization (gain or loss of electrons). A certain non-limiting example of ionizing radiation is an x-radiation. Means for delivering x-radiation to a target tissue or cell are known in the art.

In some aspects, the radiotherapy can comprise external radiotherapy, internal radiotherapy, radioimmunotherapy, or intraoperative radiation therapy (IORT). In some aspects, the external radiotherapy comprises three-dimensional conformal radiation therapy (3D-CRT), intensity modulated radiation therapy (IMRT), proton beam therapy, image-guided radiation therapy (IGRT), or stereotactic radiation therapy. In some aspects, the internal radiotherapy comprises interstitial brachytherapy, intracavitary brachytherapy, or intraluminal radiation therapy. In some aspects, the radiotherapy is administered to a primary tumor. In some aspects, the radiotherapy is administered to a metastatic tumor.

### II. Cancer Immunotherapy

In some aspects, the disclosed methods comprise administering a cancer immunotherapy as a cancer therapy to a subject or patient. Cancer immunotherapy is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Various immunotherapies are known in the art, and examples are described below.

### a Checkpoint Inhibitors and Combination Treatment

Aspects of the disclosure may include administration of immune checkpoint inhibitors or immune blockade therapies, examples of which are further described below. As disclosed herein, “checkpoint inhibitor therapy” (also “immune checkpoint blockade therapy”, “immune checkpoint therapy”, “ICT,” “checkpoint blockade immunotherapy,” or “CBI”), refers to cancer therapy comprising providing one or more immune checkpoint inhibitors to a subject suffering from or suspected of having cancer.

### 1. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some aspects, PD-1, PDL1, and PDL2 are human PD-1, PDL1 and PDL2.

In some aspects, the PD-1 inhibitor is a molecule that inhibits the binding of PD-1 to its ligand binding partners. In a specific aspect, the PD-1 ligand binding partners are PDL1 and/or PDL2. In another aspect, a PDL1 inhibitor is a molecule that inhibits the binding of PDL1 to its binding partners. In a specific aspect, PDL1 binding partners are PD-1 and/or B7-1. In another aspect, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some aspects, the PDL1 inhibitor comprises AMP-224. Nivolumab, also known as MDX-1106-04, MDX-1106, ONO-4538, BMS-936558, and OPDIVOR, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some aspects, the immune checkpoint inhibitor is a PDL 1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another aspect, the antibody competes for binding with and/or binds to the same epitope on PD-1, PDL1, or PDL2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

### 2. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO 01/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

### 3. LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8+ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some aspects, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or VH and/or VL domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4 (Supp. 1): P195 (REGN3767); Yu, X., et al., mAbs, 2019; 11:6 (LBL-007) can be used in the methods disclosed herein. These and other anti-LAG-3 antibodies useful in the claimed disclosure can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some aspects, the inhibitor comprises the heavy and light chain CDRs or VRs of an anti-LAG3 antibody. Accordingly, in one aspect, the inhibitor comprises the CDR1, CDR2, and CDR3 domains of the VH region of an anti-LAG3 antibody, and the CDR1, CDR2 and CDR3 domains of the VL region of an anti-LAG3 antibody. In another aspect, the antibody has at least about 70, 75, 80, 85, 90, 95, 97, or 99% (or any derivable range therein) variable region amino acid sequence identity with the above-mentioned antibodies.

### 4. TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4+ Th1 and CD8+ Tc1 cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+Th1-specific cell surface protein that regulates macrophage activation. Inhibitors of the disclosure may block one or more functions of TIM-3 activity.

In some aspects, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or VH and/or VL domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed disclosure can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to LAG3 also can be used.

In some aspects, the inhibitor comprises the heavy and light chain CDRs or VRs of an anti-TIM-3 antibody. Accordingly, in one aspect, the inhibitor comprises the CDR1, CDR2, and CDR3 domains of the VH region of an anti-TIM-3 antibody, and the CDR1, CDR2 and CDR3 domains of the VL region of an anti-TIM-3 antibody. In another aspect, the antibody has at least about 70, 75, 80, 85, 90, 95, 97, or 99% (or any derivable range therein) variable region amino acid sequence identity with the above-mentioned antibodies.

b. Inhibition of Co-Stimulatory Molecules

In some aspects, the immunotherapy comprises an inhibitor of a co-stimulatory molecule. In some aspects, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

c. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

d. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

### III. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

### IV. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumorinfiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

In particular aspects of the present disclosure, a cancer immunotherapy comprises treatment with ipilimumab and nivolumab in combination. In certain aspects of the present disclosure, a cancer immunotherapy comprises treatment with pembrolizumab.

### V. Chemotherapies

In some aspects, the cancer therapy to be administered to the subject comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane).

### VI. Surgery

In some aspects, the cancer therapy to be administered to the subject comprises one or more surgeries. Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

E7. Exemplary Embodiments for Determining Whether a Patient is not Likely to Benefit from Checkpoint Inhibitor Therapy

Plasma glycoproteomic biomarkers identify metastatic melanoma patients with reduced clinical benefit from checkpoint inhibitors therapy. The clinical success of immune-checkpoint inhibitors (ICI) in metastatic melanoma has confirmed the validity of therapeutic strategies that boost the immune system to counteract cancer. However, as only about half of patients derive durable clinical benefit, there is a need for assays that can identify non-responders with high accuracy, fast turnaround time and minimal invasiveness. A novel platform was utilized that combines mass spectrometry with an artificial intelligence-based data processing engine to interrogate the blood glycoproteome in melanoma patients before receiving ICI therapy. Independent training, validation and testing cohorts were leveraged to generate a model that predicts immunotherapy response (HR=2.7; P=0.026). The classifier was further validated in an independent cohort and achieved a significant separation of responders and non-responders (HR=5.6; P=0.027). To understand how glycosylation of circulating proteins may affect response to treatment, changes in glycosylation structure were analyzed and a fucosylation signature was discovered in non-responders. A fucosylation-based predictor was developed that stratified effectively patients, supporting the hypothesis that this protein modification directly impacts ICI efficacy. The data demonstrates the utility of the prediction of ICI benefit in patients with malignant melanoma.

Over the last decade, ICI therapeutics have significantly advanced the clinical management and outcome of patients with a range of malignancies, including metastatic melanoma; yet, only 30-40% of patients obtain sustained clinical benefit from single agent ICI therapy such as pembrolizumab or nivolumab. While combination treatment of nivolumab with ipilimumab has resulted in higher response rates of up to 60%, this is also associated with an incidence of 55% of grade 3-to-4 side effects. Therefore, considerable efforts have been expended to identify predictive biomarkers as companion diagnostics for the early determination of patients who are unlikely to benefit from ICI treatment or may experience severe adverse events, to guide alternative therapies. Assessment of PD-L1 expression in the tumor microenvironment and quantification of tumor mutational burden have found limited utility as indicators of durable clinical benefits. Likewise, a number of other genomic and transcriptomic markers have been investigated as predictors of response of metastatic melanoma to ICI therapies, including HLA-I genotype, neoantigen load and T cell repertoire. In addition, gene expression signatures like immune-predictive score (IMPRES) and IFN-γ-responsive genes expressed in tumors or tumor immune microenvironment (TiME) have been proposed as predictors of ICI response in metastatic melanoma. While these approaches are fruitful, they have yet not demonstrated broad clinical utility as there is debate around their consistency and reproducibility across cohorts.

The utility of molecular tumor profiling is also limited by the heterogeneity of tumor samples and the availability of adequate tissue obtained through invasive procedures. Liquid biopsies have emerged as a viable alternative to monitor treatment response as they are minimally invasive, convenient for serial sampling, and informative of functional states of immune cells modulated by ICI. Whereas peripheral immune cells and plasma factors contribute to antitumor responses modulated by ICI, previous studies focused on small groups of proteins. Moreover, the impact of post-translational modifications of circulating proteins on ICI response has not been evaluated in a systematic and scalable way to date.

The plasma glycoproteome of metastatic melanoma patients was interrogated before receiving ICI treatment and using a novel analytical platform that employed machine learning methods to analyze data generated by targeted mass spectrometry. Glycopeptide markers differentially expressed in patients with a short or durable response were identified and used to build a predictor of response to therapy. It should be noted that a durable response in this context means a relatively prolonged response to immunotherapy that is associated with a decreased progression of the melanoma and an improved overall survivability, whereas a short response means a relatively poor response to immunotherapy that is associated with no decreased progression of the melanoma and no improvement to overall survivability.

A discovery cohort of 202 patients was recruited as part of studies conducted at the Massachusetts General Hospital (MGH) in which patients with metastatic melanoma (stage IV) were treated with first or second-line anti-PD-1 monotherapy or anti-PD-1/anti-CTLA4 combination therapy. Pre-treatment samples were collected under the Institutional Review Board protocols of the Massachusetts General Hospital (protocols 12-488 and 11-181). In this context, pre-treatment samples refer to samples obtained from subject who have not yet received the immunotherapy for this clinical study. Written informed consent was obtained from each patient. Responses to immunotherapy were assessed for overall survival (OS). A validation cohort of 27 patients was recruited as part of studies conducted at the University of South Australia (UniSA) in which patients with metastatic melanoma were treated with first or second-line anti-PD-1 monotherapy or anti-PD-1/anti-CTLA4 combination therapy. Hereinafter, the “discovery cohort” will refer to the samples collected at MGH and the “independent validation cohort” or “external validation cohort” will refer to the samples collected at the UniSA. Written informed consent was obtained from each patient. Plasma was stored at −80° C. and thawed at the time of the glycoproteomic analysis.

Pooled human plasma (MilliporeSigma, St. Louis, MO) was used for quality control, assay normalization and calibration purposes. Dithiothreitol (DTT) and iodoacetamide (IAA) were purchased from MilliporeSigma (St. Louis, MO). Mass spectrometry grade trypsin/Lys-C protease mix, formic acid, and acetonitrile were purchased from Thermo Fisher Scientific (Waltham, MA).

Plasma samples were thermally denatured at 100° C. for 5 minutes before they were reduced with DTT, alkylated with IAA, quenched with DTT, and followed by digestion with trypsin/Lys-C protease mix in a water bath at 37° C. for 18 hours. To quench the digestion, formic acid was added to each sample after incubation to a final concentration of 1% (v/v). Digested plasma samples were injected into an Agilent 6495C triple quadrupole mass spectrometer equipped with an Agilent 1290 Infinity ultra-high-pressure liquid chromatography (UHPLC) system and a Waters (Milford, MA) ACQUITY Peptide HSS T3 Column (2.1 mm internal diameter×150 mm length, 1.8 μm particle size). Separation of the peptides and glycopeptides was performed using a 49-min binary gradient. The aqueous mobile phase A was 0.1% formic acid in water (v/v), and the organic mobile phase B was 0.1% formic acid in acetonitrile (v/v). The flow rate was set at 0.5 mL/min. Electrospray ionization was used as the ionization source and was operated in positive ion mode. The triple quadrupole MS was operated in dynamic multiple reaction monitoring (dMRM) mode, with modifications and improvements from a previous method. Samples were injected in a randomized fashion with regard to clinical parameters, and reference pooled plasma samples were injected interspersed with test samples to allow for correction of within-run drift of baseline signal.

PB-NET, a peak integration software that had been developed in-house, was used to integrate peaks and obtain raw abundances for peptides and glycopeptides. Raw abundance for peptide markers was normalized by using spiked-in heavy isotope-labeled internal standards with known peptide concentrations for each protein. Relative abundance was determined for a glycopeptide by calculating the quotient of a raw abundance signal intensity of the glycopeptide and the raw abundance of a corresponding non-glycosylated peptide from the same protein. For glycopeptides having the same peptide sequence with more than two glycan types identified at a given glycosylation site, site occupancy was determined for a particular glycopeptide by calculating its fractional abundance across all glycan types observed at that site. It should be noted that each glycan type represents a unique combination of saccharide units that is attached to a same glycosylation site of a particular peptide sequence. For each glycopeptide biomarker, the product of its site occupancy or relative abundance and corresponding peptide concentration was used to calculate approximate glycopeptide concentration, also referred to as normalized abundance. Concentration was determined for 521 glycopeptides, 443 of which are based on site occupancy and 78 on relative abundance, and for 75 peptides, totaling 596 unique concentration-normalized biomarkers. Relative abundance was determined for 532 unique glycopeptides. Univariate age- and sex-adjusted Cox regression with respect to overall survival % (OS) was performed using both relative abundance-normalized features and concentration-normalized features to determine a set of glycopeptides and non-glycosylated peptides that are significantly associated with OS.

The sample set was randomly divided and stratified by immunotherapy regimen, melanoma subtype, and early failure (progressed or died within 6 months of treatment start)/sustained control (progression and death-free within 3 years of treatment start) status. 40% of the sample set was used as a training set, 30% was used as a validation set on which to tune model hyperparameters, and the remaining 30% was used as an independent testing set. Allocation of samples into the three sets (training, validation, and test) was sufficiently balanced across all demographic and clinical covariates listed in Table 22A (confirmed by Chi-squared, Fisher's Exact, Student t, or Wilcoxon rank-based tests as appropriate). A five-fold repeated cross-validated LASSO-regularized Cox regression model was employed on the training set, yielding a set of 14 biomarkers, including 13 glycopeptides and one non-glycosylated peptide (Tables 23A-D). A risk score threshold by which to divide the cohort into “likely ICI responders” and “non-responders” was chosen via Harrell's c-index to optimize sensitivity for non-response in the validation set. The identical threshold was used in the independent test set and in the external validation cohort. All analyses were conducted in R version 4.2.2 (Vienna, Austria).

TABLE 22A
Demographic and clinical covariates in the discovery cohort. Counts are followed by the
appropriate column-wise percentage, while continuous variables are summarized by medians
and either IQRs or, for time to event variables, 95% confidence limits (NR = not reached).

| Variable | Full cohort | Training set | Validation set | Test set |
|---|---|---|---|---|
| Sample size | 202 | 79 (39) | 59 (29) | 64 (32) |
| Female sex | 63 (31) | 27 (34) | 16 (27) | 20 (31) |
| Age, yrs. (continuous) | 65 (57, 73) | 67 (57, 72) | 64 (56, 74.5) | 63.5 (57, 72.2) |
| Current ICI treatment | | | | |
| Pembrolizumab monotherapy | 109 (54) | 43 (54) | 32 (54) | 34 (53) |
| Nivolumab with/without | 93 (46) | 36 (46) | 27 (46) | 30 (47) |
| ipilimumab combination | | | | |
| Survival-related events | | | | |
| Progression (PFS) event | 145 (72) | 57 (72) | 40 (68) | 48 (75) |
| Death (OS) event | 113 (56) | 42 (53) | 32 (54) | 39 (61) |
| Next treatment event | 83 (41) | 30 (38) | 22 (37) | 31 (48) |
| Time to event, mos. | | | | |
| (continuous) | | | | |
| Progression | 5.5 (3.0, 9.9) | 6.9 (3.3, 23.9) | 8.4 (2.7, 30.4) | 3.2 (2.5, 9.3) |
| Death | 40.1 (27.3, 59.0) | 50.4 (30.3, NR) | 36.1 (14.3, NR) | 28.8 (16.2, NR) |
| BRAF status | | | | |
| Negative | 67 (33) | 22 (28) | 18 (31) | 27 (42) |
| V600 | 57 (28) | 20 (25) | 16 (27) | 21 (33) |
| Non-V600 | 9 (4) | 2 (3) | 2 (3) | 5 (8) |
| Mutant | 1 (0) | 0 (0) | 0 (0) | 1 (2) |
| Positive/wild type | 121 (60) | 49 (62) | 35 (59) | 37 (58) |
| Missing | 14 (7) | 8 (10) | 6 (10) | 0 (0) |
| LDH, units/L (continuous) | 206 (167, 281) | 218 (168.8, 284.8) | 206 (167, 274) | 191.5 (164.8, 276.8) |
| LDH (categorical) | | | | |
| <ULN | 105 (52) | 36 (46) | 29 (49) | 40 (62) |
| 1-2xULN | 63 (31) | 28 (35) | 22 (37) | 13 (20) |
| >2xULN | 27 (13) | 12 (15) | 6 (10) | 9 (14) |
| Missing | 7 (3) | 3 (4) | 2 (3) | 2 (3) |
| M Stage | | | | |
| M0 | 16 (8) | 7 (9) | 5 (8) | 4 (6) |
| M1 | 186 (92) | 72 (91) | 54 (92) | 60 (94) |
| M1a | 9 (4) | 3 (4) | 2 (3) | 4 (6) |
| M1b | 31 (15) | 17 (22) | 5 (8) | 9 (14) |
| M1c | 84 (42) | 28 (35) | 30 (51) | 26 (41) |
| M1d | 62 (31) | 24 (30) | 17 (29) | 21 (33) |
| ECOG performance status | | | | |
| 0 | 119 (59) | 49 (62) | 34 (58) | 36 (56) |
| 1 | 67 (33) | 24 (30) | 19 (32) | 24 (38) |
| ≥2 | 14 (7) | 5 (6) | 6 (10) | 3 (5) |
| Missing | 2 (1) | 1 (1) | 0 (0) | 1 (2) |
| Melanoma subtype | | | | |
| Cutaneous | 127 (63) | 51 (65) | 37 (63) | 39 (61) |
| Mucosal | 20 (10) | 8 (10) | 5 (8) | 7 (11) |
| Uveal | 15 (7) | 6 (8) | 5 (8) | 4 (6) |
| Acral | 5 (2) | 1 (1) | 1 (2) | 3 (5) |
| Unknown primary | 35 (17) | 13 (16) | 11 (19) | 11 (17) |
| Line of therapy | | | | |
| First-line | 147 (73) | 61 (77) | 44 (75) | 42 (66) |
| Second-line or later | 54 (27) | 17 (22) | 15 (25) | 22 (34) |
| Missing | 1 (0) | 1 (1) | 0 (0) | 0 (0) |

TABLE 22B
Demographic and clinical covariates in the discovery cohort stratified by classifier
prediction. Counts are followed by the appropriate column-wise percentage,
while continuous variables are summarized by medians and either IQRs or, for
time to event variables, 95% confidence limits (NR = not reached).

| Variable | Full cohort | Likely to benefit | Unlikely to benefit |
|---|---|---|---|
| Sample size | 202 | 179 (89) | 23 (11) |
| Female sex | 63 (31) | 56 (31) | 7 (30) |
| Age, yrs. (continuous) | 65 (57, 73) | 65 (56.5, 73) | 68 (59.5, 78.5) |
| Current ICI treatment | | | |
| Pembrolizumab monotherapy | 109 (54) | 98 (55) | 11 (48) |
| Nivolumab with/without | 93 (46) | 81 (45) | 12 (52) |
| ipilimumab combination | | | |
| Survival-related events | | | |
| Progression (PFS) event | 145 (72) | 124 (69) | 23 (91) |
| Death (OS) event | 113 (56) | 92 (51) | 23 (91) |
| Next treatment event | 83 (41) | 76 (42) | 7 (30) |
| Time to event, mos. | | | |
| (continuous) | | | |
| Progression | 5.5 (3.0, 9.9) | 7.6 (4.0, 16.9) | 2.1 (1.3, 3.0) |
| Death | 40.1 (27.3, 59.0) | 54.3 (37.9, NR) | 3.7 (2.4, 10.8) |
| BRAF status | | | |
| Negative | 67 (33) | 63 (35) | 4 (17) |
| V600 | 57 (28) | 53 (30) | 4 (17) |
| Non-V600 | 9 (4) | 9 (5) | 0 (0) |
| Mutant | 1 (0) | 1 (1) | 0 (0) |
| Positive/wild type | 121 (60) | 104 (58) | 17 (74) |
| Missing | 14 (7) | 12 (7) | 2 (9) |
| LDH, units/L (continuous) | 206 (167, 281) | 200 (166, 266.8) | 357 (210, 792) |
| LDH (categorical) | | | |
| <ULN | 105 (52) | 99 (55) | 6 (26) |
| 1-2xULN | 63 (31) | 57 (32) | 6 (26) |
| >2xULN | 27 (13) | 16 (9) | 11 (48) |
| Missing | 7 (3) | 7 (4) | 0 (0) |
| M Stage | | | |
| M0 | 16 (8) | 15 (8) | 1 (4) |
| M1 | 186 (92) | 164 (92) | 22 (96) |
| M1a | 9 (4) | 8 (4) | 1 (4) |
| M1b | 31 (15) | 30 (17) | 1 (4) |
| M1c | 84 (42) | 73 (41) | 11 (48) |
| M1d | 62 (31) | 53 (30) | 9 (39) |
| ECOG performance status | | | |
| 0 | 119 (59) | 113 (63) | 6 (26) |
| 1 | 67 (33) | 58 (32) | 9 (39) |
| ≥2 | 14 (7) | 8 (4) | 6 (26) |
| Missing | 2 (1) | 0 (0) | 2 (9) |
| Melanoma subtype | | | |
| Cutaneous | 127 (63) | 116 (65) | 11 (48) |
| Mucosal | 20 (10) | 18 (10) | 2 (9) |
| Uveal | 15 (7) | 12 (7) | 3 (13) |
| Acral | 5 (2) | 5 (3) | 0 (0) |
| Unknown primary | 35 (17) | 28 (16) | 7 (30) |
| Line of therapy | | | |
| First-line | 147 (73) | 131 (73) | 16 (70) |
| Second-line or later | 54 (27) | 48 (27) | 6 (26) |
| Missing | 1 (0) | 0 (0) | 1 (4) |

In some embodiments, provided herein are methods for determining whether a subject diagnosed with melanoma will benefit from immunotherapy comprising detecting one or more biomarkers. In some embodiments, the one or more biomarkers comprise one or more glycopeptides. In some embodiments, the one or more biomarkers comprises one or more peptide structures set forth in Table 23A. In some embodiments, the method comprises detecting one or more peptides comprising a sequence set forth in SEQ ID NOs: 95-108. In some embodiments, the peptides of Table 23A can comprise a glycan with the symbol structure or composition noted in Table 23C.

TABLE 23A
Peptide Structures Associated with Likelihood of Benefit of
Immunotherapy in Melanoma Patients

| | | | | Linking | Linking | |
|---|---|---|---|---|---|---|
| | | | | Site | Site | |
| | | | | Position | Position | |
| (Peptide) | | | | within | within | Glycan |
| SEQ ID | | Uniprot | | Protein | Peptide | Structure |
| NO. | PS-NAME | ID | Peptide sequence | Sequence | Sequence | GL NO. |
| 4495 | A1AT_107_5412 | P01009 | ADTHDEILEGLNFNL | 107 | 14 | 5412 |
| | | | TEIPEAQIHEGFQELL | | | |
| | | | R | | | |
| 4596 | A1AT_107_6503 | P01009 | ADTHDEILEGLNFNL | 107 | 14 | 6503 |
| | | | TEIPEAQIHEGFQELL | | | |
| | | | R | | | |
| 4697 | AACT_106_6503 | P01011 | FNLTETSEAEIHQSF | 106 | 2 | 6503 |
| | | | QHLLR | | | |
| 4798 | AGP12_72MC_6503 | P02763 | SVQEIQATFFYFTPN | 72 | 15 | 6503 |
| | | or | KTEDTIFLR | | | |
| | | P19652 | | | | |
| 4899 | AGP12_72MC_7602 | P02763 | SVQEIQATFFYFTPN | 72 | 15 | 7602 |
| | | or | KTEDTIFLR | | | |
| | | P19652 | | | | |
| 49100 | APOB_3411_5401 | P04114 | FVEGSHNSTVSLTTK | 3411 | 7 | 5401 |
| 50101 | APOM_135_NONG | O95445 | TELFSSSCPGGIMLN | N/A | N/A | N/A |
| | LYCOSYLATED | | ETGQGYQR | | | |
| 51102 | CFAH_882_5411 | P08603 | IPCSQPPQIEHGTIN | 882 | 15 | 5411 |
| | | | SSR | | | |
| 52103 | CLUS_291_5401 | P10909 | HNSTGCLR | 291 | 2 | 5401 |
| 53104 | IGG1_297_5412 | P01857 | EEQYNSTYR | 180 | 5 | 5412 |
| 54105 | IGG2_297_5401 | P01859 | EEQFNSTFR | 176 | 5 | 5401 |
| 55106 | KLKB1_308_5401 | P03952 | IYPGVDFGGEELNVT | 308 | 13 | 5401 |
| | | | FVK | | | |
| 56107 | KLKB1_494_5410 | P03952 | LQAPLNYTEFQKPIC | 494 | 6 | 5410 |
| | | | LPSK | | | |
| 57108 | FINC_SYTITGLQPGT | P02751 | SYTITGLQPGTDYK | N/A | N/A | N/A |
| | DYK | | | | | |

TABLE 23B
LC-MRM-MS parameters for peptide structures associated with Patients Having
Melanoma that are Unlikely or Likely to Benefit with Immunotherapy

| (Peptide) | | | Collision | 1st | 1st | 1st | 2nd |
|---|---|---|---|---|---|---|---|
| SEQ | Monoisotopic | RT | Energy | Precursor | Precursor | Product | Product |
| ID NO. | mass (g/mole) | (min) | (V) | m/z | charge | m/z | m/z |
| 4495 | 6041.646782 | 42.8 | 30 | 1209.8 | 5 | 366.1 | 1299 |
| 4596 | 6551.816474 | 43.3 | 33 | 1311.8 | 5 | 366.1 | 1299 |
| 4697 | 5260.186906 | 37.9 | 30 | 1053.4 | 5 | 274.1 | N/A |
| 4798 | 5755.448988 | 41 | 28 | 1152.7 | 5 | 366.1 | 1550.3 |
| 4899 | 5829.485766 | 40.5 | 25 | 1458.9 | 4 | 366.1 | 1550.3 |
| 49100 | 3519.476794 | 12.5 | 30 | 1174.2 | 3 | 366.1 | N/A |
| 50101 | 2531.142012 | 31.1 | 28 | 845.3 | 3 | 1052.5 | 523.3 |
| 51102 | 4079.71446 | 14.8 | 25 | 1021.4 | 4 | 366.1 | N/A |
| 52103 | 2857.106354 | 4.9 | 24 | 953.4 | 3 | 366.1 | N/A |
| 53104 | 3539.335 | 8.6 | 40 | 1181.1 | 3 | 204.1 | N/A |
| 54105 | 3070.191856 | 14 | 30 | 1024.7 | 3 | 204.1 | 1360.6 |
| 55106 | 3896.675884 | 37 | 20 | 975.7 | 4 | 366.1 | N/A |
| 56107 | 4014.816354 | 30.4 | 20 | 1004.7 | 4 | 366.1 | N/A |
| 57108 | 1542.756554 | 22.7 | 22 | 772.4 | 2 | 680.3 | 978.4 |

TABLE 23C
Glycan symbol structure and glycan composition that corresponds to the
glycan structure GL NO in Table 23A

| Glycan Structure GL | Glycan Symbol | |
|---|---|---|
| NO. | Structure | Glycan Composition |
| 5401 | 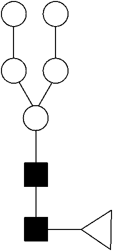 | Hex(5)HexNAc(4)Fuc(0)NeuAc(1) |
| 5410 | 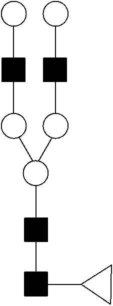 | Hex(5)HexNAc(4)Fuc(1)NeuAc(0) |
| 5411 | 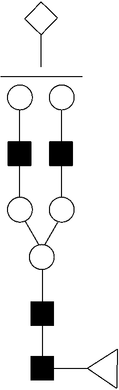 | Hex(5)HexNAc(4)Fuc(1)NeuAc(1) |
| 5412 | 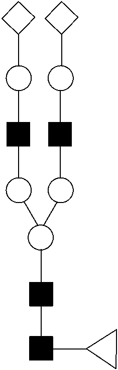 | Hex(5)HexNAc(4)Fuc(1)NeuAc(2) |
| 6503 | 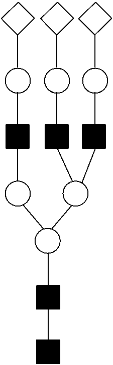 | Hex(6)HexNAc(5)Fuc(0)NeuAc(3) |
| 7602 | 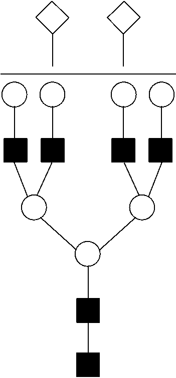 | Hex(7)HexNAc(6)Fuc(0)NeuAc(2) |

Legend for Table 19

[symbol]

Glc

[symbol]

Gal

[symbol]

Man

[symbol]

Fuc

[symbol]

NeuSAc

[symbol]

GlcNAc

[symbol]

GalNAc

[symbol]

ManNAc

TABLE 23D
Coefficients for each marker used in the regression models

| (Peptide) | | | |
|---|---|---|---|
| SEQ ID | | Uniprot | |
| NO. | PS-NAME | ID | Coeficients |
| 4495 | A1AT_107_5412 | P01009 | 0.164329 |
| 4596 | A1AT_107_6503 | P01009 | 0.001652 |
| 4697 | AACT_106_6503 | P01011 | 0.021662 |
| 4798 | AGP12_72MC_6503 | P02763 | 0.079666 |
| | | or | |
| | | P19652 | |
| 4899 | AGP12_72MC_7602 | P02763 | 0.076526 |
| | | or | |
| | | P19652 | |
| 49100 | APOB_3411_5401 | P04114 | −0.01875 |
| 50101 | APOM_135_{—} | O95445 | −0.04415 |
| | NONGLYCOSYLATED | | |
| 51102 | CFAH_882_5411 | P08603 | −0.02179 |
| 52103 | CLUS_291_5401 | P10909 | −0.16244 |
| 53104 | IGG1_297_5412 | P01857 | −0.02911 |
| 54105 | IGG2_297_5401 | P01859 | 0.078805 |
| 55106 | KLKB1_308_5401 | P03952 | −0.08128 |
| 56107 | KLKB1_494_5410 | P03952 | 0.138484 |
| 57108 | FINC_{—} | P02751 | −0.07506 |
| | SYTITGLQPGTDYK | | |

Glycopeptides with differences in relative abundance in relation to OS were analyzed by type of glycans. Asparagine (N)-linked glycans and O-linked glycopeptides were further separated based on the number of hexose, N-acetylhexosamine, fucose, or sialic acid units. A site-specific monomer weight feature for N-glycopeptides was calculated by determining the average number of a specific monosaccharide at a given glycosylation site, weighted by glycan species site occupancy. A five-fold repeated cross-validated LASSO-regularized Cox regression model based only on fucose-dependent engineered features that achieved FDR<0.05 in univariate age- and sex-adjusted Cox regression was developed, and a risk score threshold was chosen using the same method as described above. The same training, validation, and test set were used as in the other cross-validated Cox model described above.

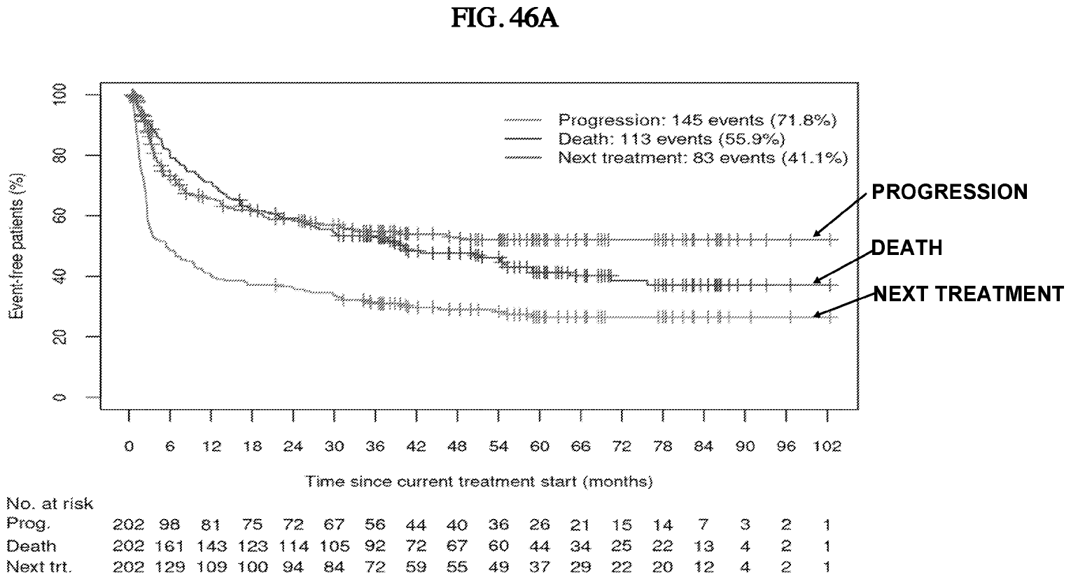

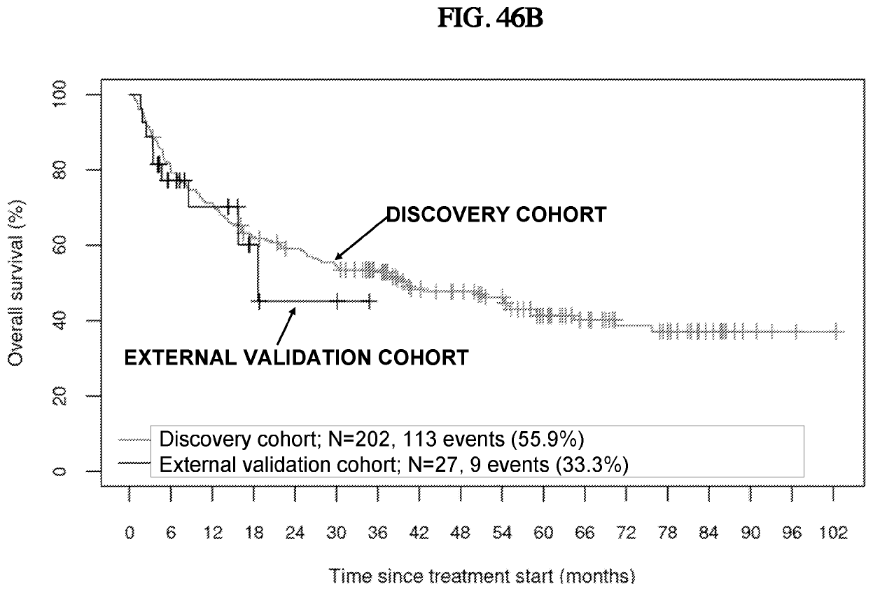

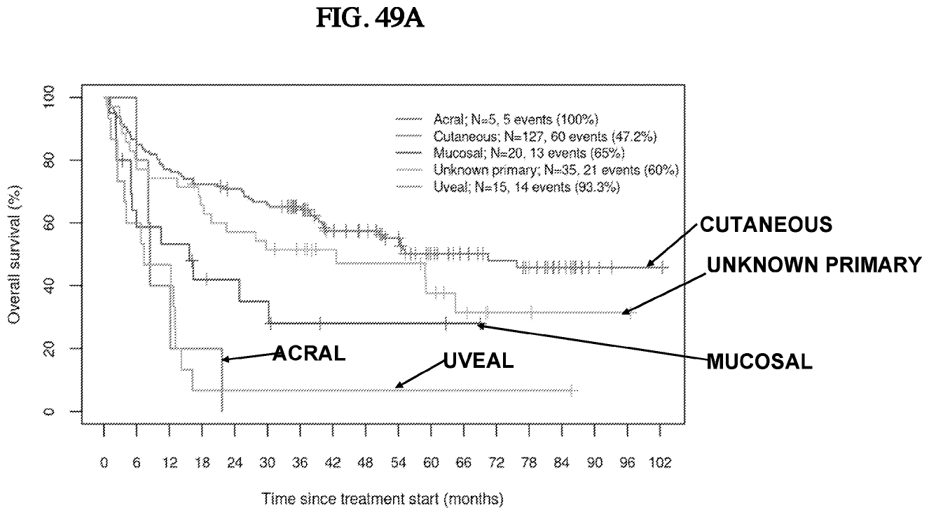

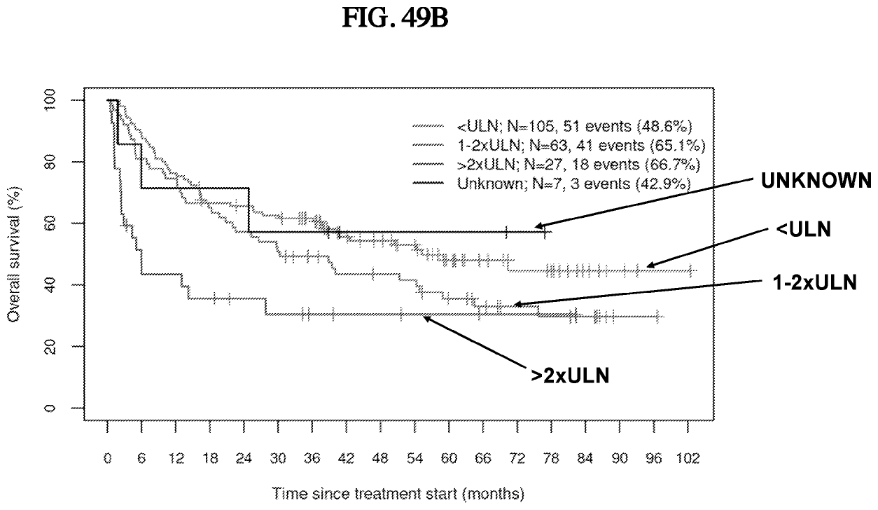

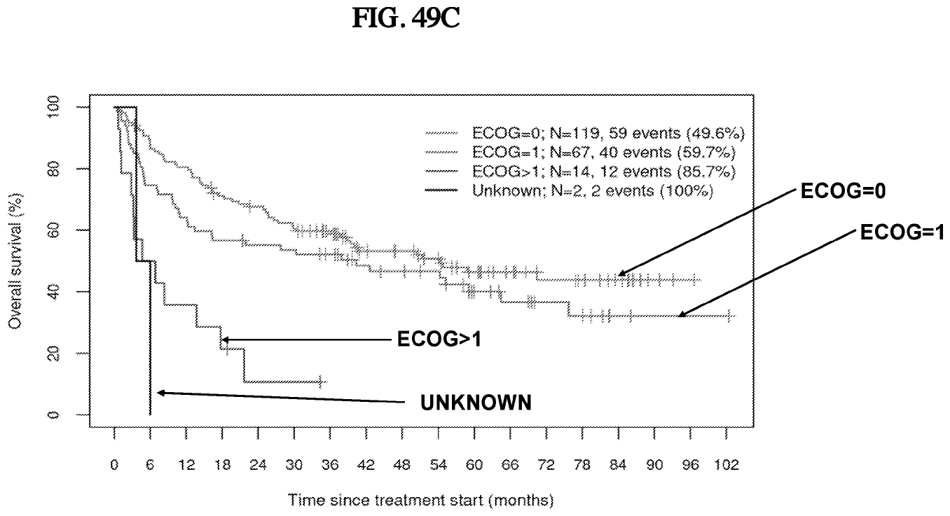

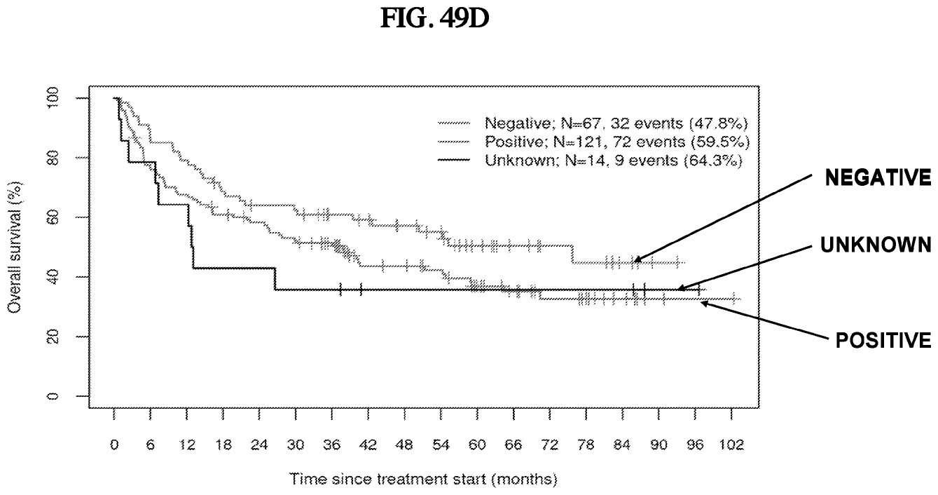

Responses to immunotherapy were assessed for overall survival (OS). The discovery cohort is composed of 69% male with a median age of 65 (IQR: 57, 73). 73% of patients were to undergo first-line therapy at time of sample collection, with 72% of patients experiencing a progression, 41% having a time recorded at which an additional treatment regimen was initiated, and 56% having a recorded death. Median time to progression for this cohort was 5.5 months (95% CI: 3.0, 9.9), while median time to death was over three years, at 40.1 months (95% CI: 27.3, 59.0) (FIG. 46). 63% of patients had cutaneous melanoma and 17% had melanoma of unknown primary, while the remainder were either characterized as mucosal, uveal, or acral subtype. At the start of treatment, 73% of the cohort was staged as either M1c or M1d, 60% of patients were wild type BRAF, 44% had lactate dehydrogenase (LDH, units/L) value that exceeded the upper limit of normal (ULN), and 40% had an ECOG performance status above 0 (Table 22A). As expected, patients with non-cutaneous melanoma, higher LDH, higher ECOG performance status, or are BRAF mutants exhibit shorter median time to death (FIGS. 49A-D). The ECOG Performance Status Scale is a standard criteria that describes a patient's level of functioning in terms of their ability to care for themself, daily activity, and physical ability (walking, working, etc.). A higher ECOG value corresponds to a patient having a more limited daily living ability whereas a lower ECOG value (e.g., 0) corresponds to a more active patient able to carry on daily activities with fewer or no restriction.

The independent validation cohort used for additional confirmation of the predictor is composed of 70% male with a median age of 71 (IQR: 66, 81) as shown in Table 24. 85% of patients were to undergo pembrolizumab monotherapy, with 37% of patients experiencing a progression and 33% having a recorded death. Best overall response was determined, with 38% of the cohort having at least a partial response to therapy. Median time to progression for this cohort was not reached (95% CI: 9.9, NR) while median time to death was 18.6 months (95% CI: 15.8, NR) (Table 24). While this cohort has less follow-up than the discovery cohort, the distributions are comparable (FIG. 46B).

TABLE 24
Demographic and clinical covariates in the independent validation
cohort. Counts are followed by the appropriate column-wise
percentage, while continuous variables are summarized by
medians and either IQRs or, for time to event variables,
95% confidence limits (NR = not reached).

| Variable | Full cohort |
|---|---|
| Sample size | 27 |
| Female sex | 8 (30) |
| Age, yrs. (continuous) | 71 (66, 81) |
| Current ICI treatment | |
| Pembrolizumab monotherapy | 23 (85) |
| Ipilimumab/nivolumab combination | 4 (15) |
| Survival-related events | |
| Progression (PFS) event | 10 (37) |
| Death (OS) event | 9 (33) |
| Time to event, mos. | |
| (continuous) | |
| Progression | NR (9.9, NR) |
| Death | 18.6 (15.8, NR) |
| Best overall response | |
| Complete response | 5 (19) |
| Partial response | 5 (19) |
| Stable disease | 6 (22) |
| Progressive disease | 9 (33) |
| Missing | 2 (7) |

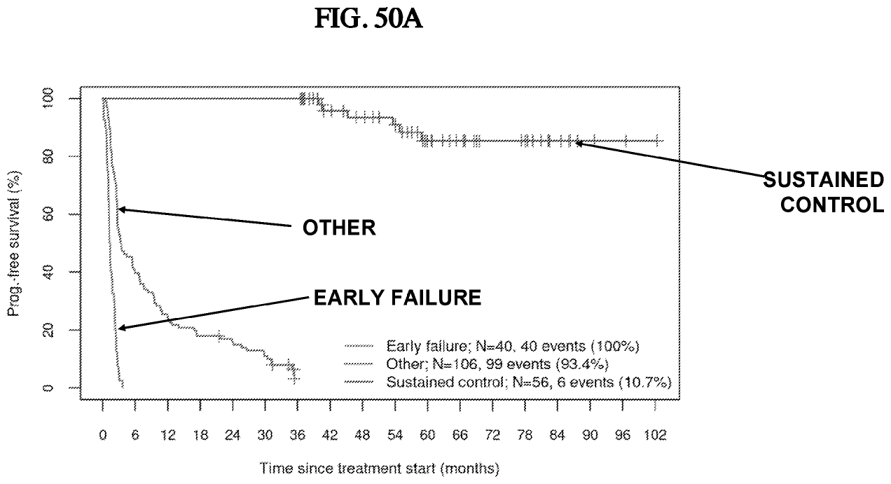

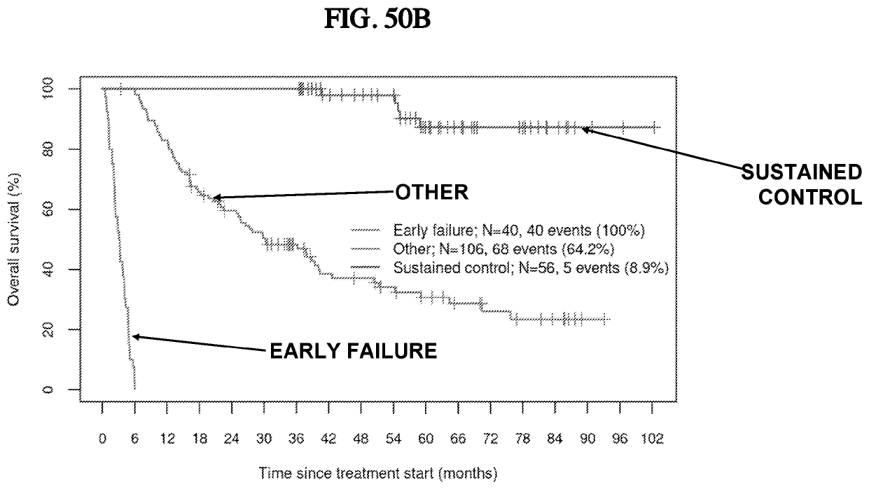

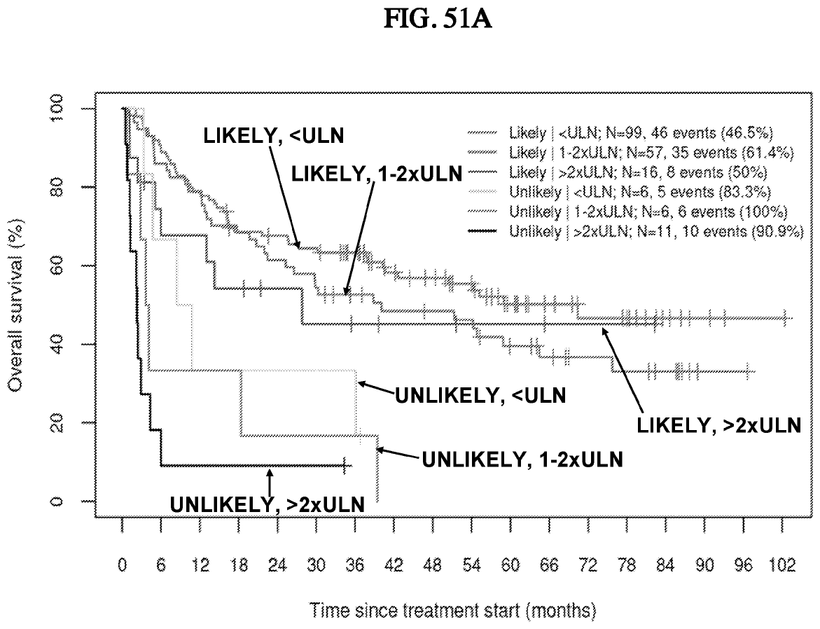

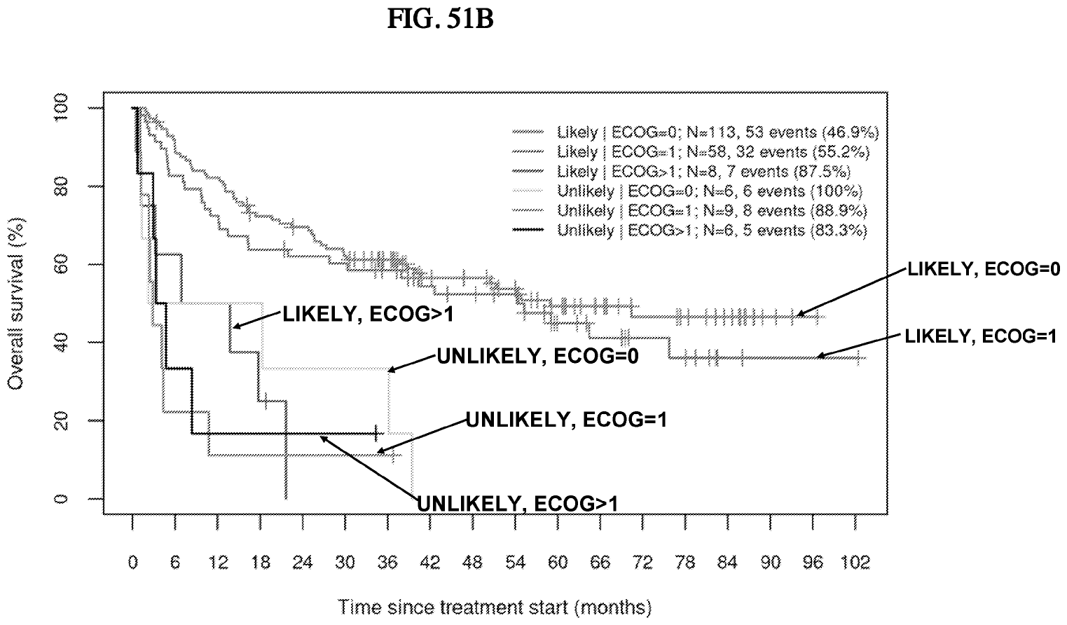

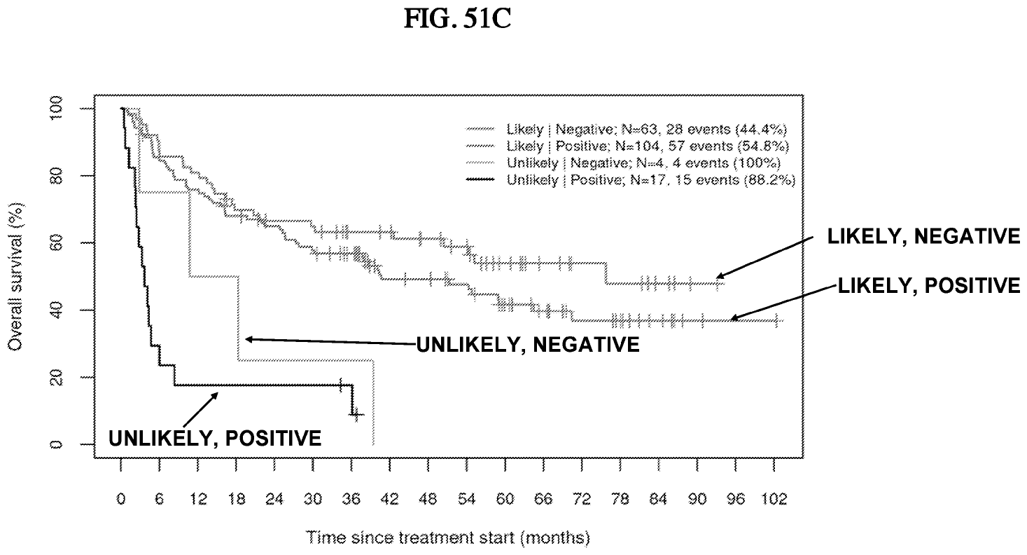

The identification of glycopeptide biomarkers associated with overall survival and early failure was described. Peptides and glycopeptides derived from 75 plasma proteins were quantified and univariate age- and sex-adjusted Cox regression were applied to identify biomarkers associated with OS. The label “early failure” (EF) was assigned to patients who progressed and died within 6 months of starting ICI treatment (n=40), “sustained control” (SC) was assigned to patients who neither progressed nor died in the first three years after ICI treatment start (n=56), and “other” was assigned otherwise (n=106) (FIGS. 50A-B). When representing 143 differentially expressed biomarkers between the EF and SC groups at FDR<0.05 in a hierarchically-clustered heatmap, drastic differences were observed in their glycoproteomic profile.

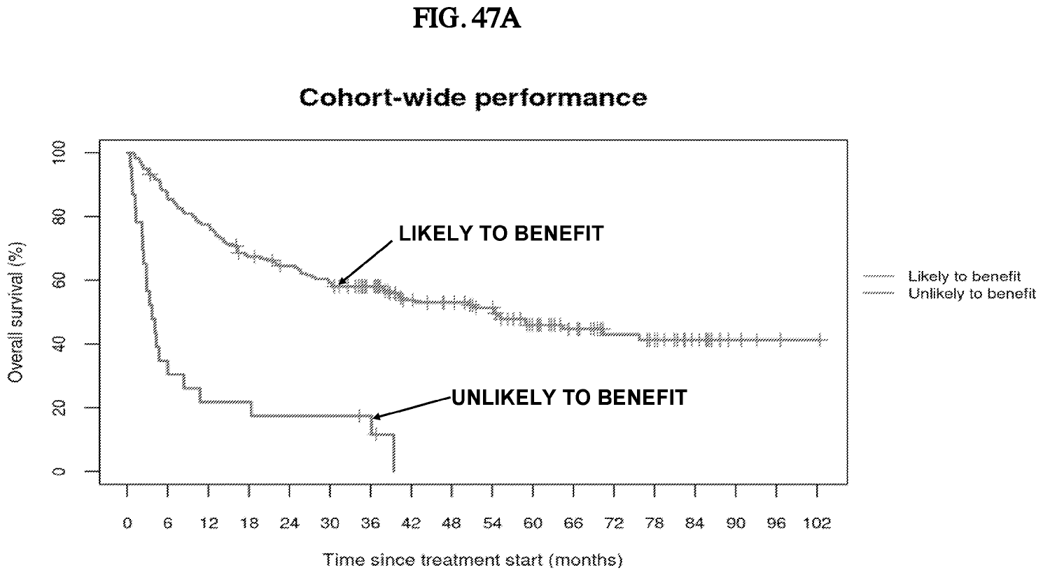

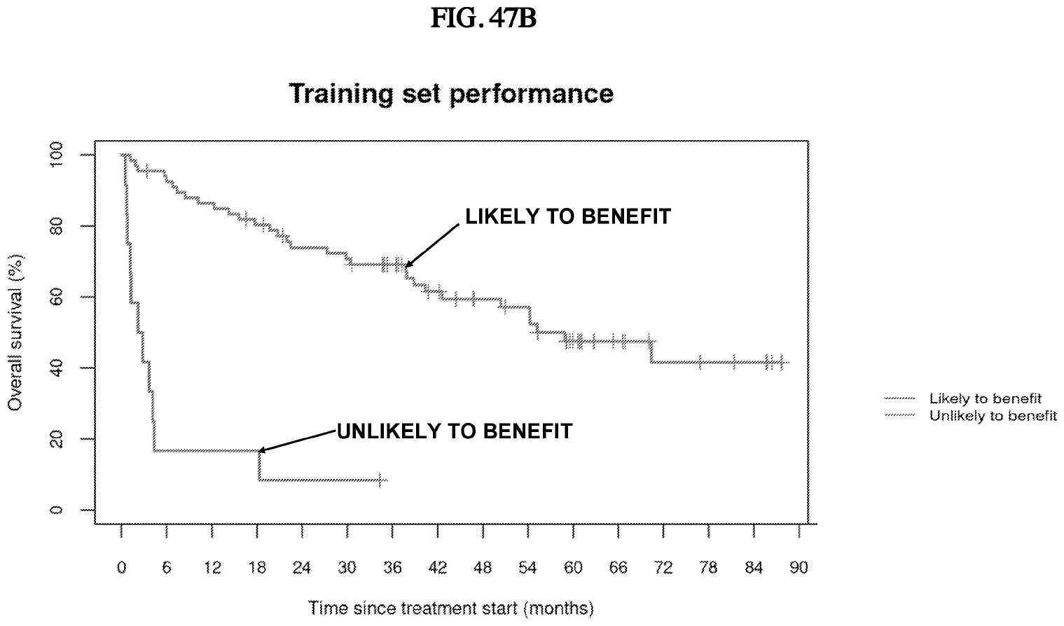

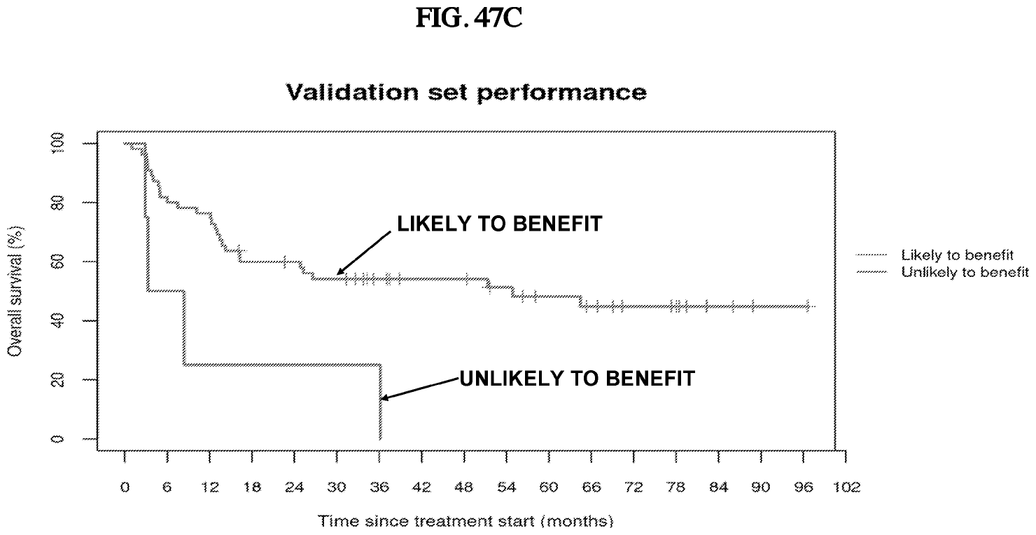

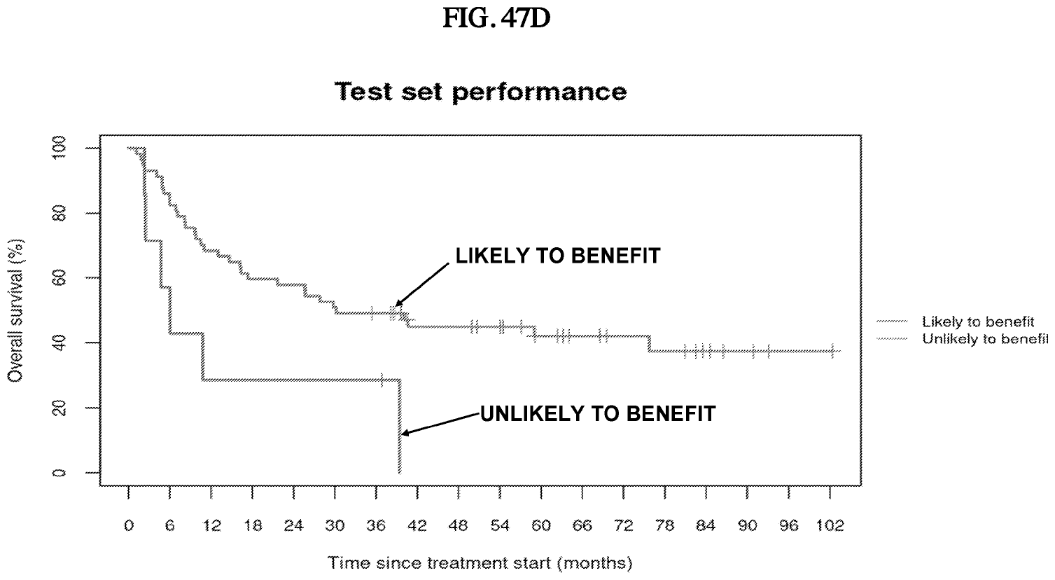

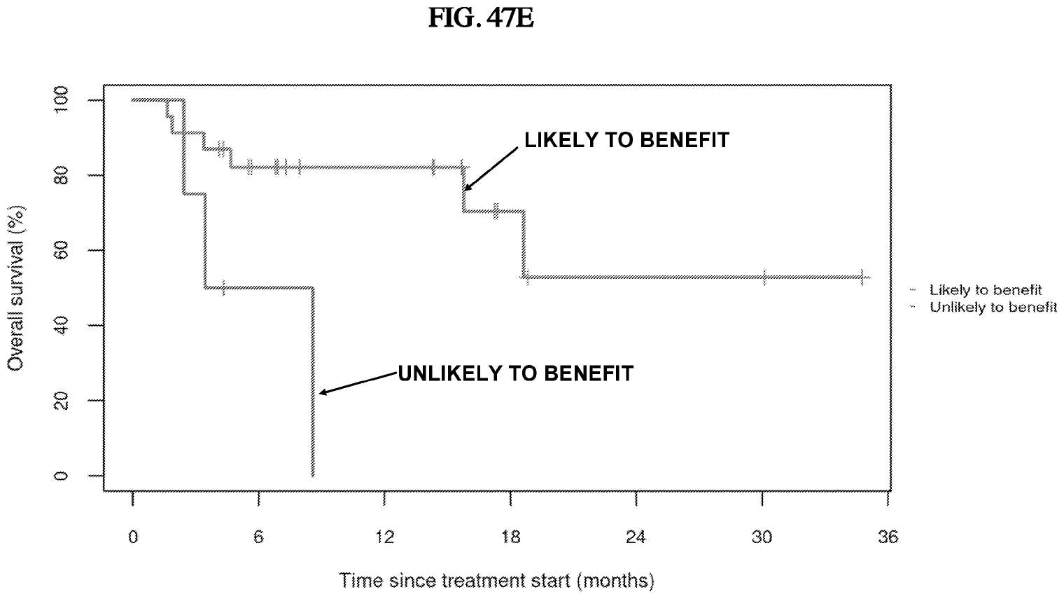

The generation of a classifier to predict ICI benefit was described. A LASSO-regularized Cox-based classifier for prediction of individuals likely to benefit from ICI therapy was generated: from 64 biomarker features associated with OS (FDR<0.05), 14 were retained (13 glycopeptides and one non-glycosylated peptide), and achieved an unadjusted HR=5.1 (P=7.6×10⁻¹¹) in the full discovery cohort, HR=10.3 (P=4.5×10⁻⁹) in the training set, HR=3.9 (P=0.012) in the validation set, and HR=2.7 (P=0.026) in the test set (Table 25, FIGS. 47A-D). In the full discovery cohort, 92 of the 179 patients classified as likely to benefit from ICI therapy died (51%), whereas 21 of 23 patients classified as unlikely to benefit died (91%), suggesting the classifier has a high sensitivity to detect likely non-responders. In the independent test set, the proportions shift to 58% and 86%, respectively.

TABLE 25
Performance of repeated five-fold cross-validated LASSO-regularized Cox
regression-based classifier using 14 concentration-normalized features

| | | | | |
|---|---|---|---|---|
| Classifier prediction | Events/N | Median OS (95% CI) | HR (95% CI) | P-value |

Full discovery cohort (n = 202)

| Likely to benefit | 92/179 | 54.3 (37.9, NR) | Reference | |
|---|---|---|---|---|
| Unlikely to benefit | 21/23 | 3.7 (2.4, 10.8) | 5.1 (3.1, 8.4) | 7.6 × 10⁻¹¹ |

Discovery: training set (n = 79)

| Likely to benefit | 31/67 | 55.2 (42.6, NR) | Reference | |
|---|---|---|---|---|
| Unlikely to benefit | 11/12 | 2.5 (1.2, NR) | 10.3 (4.7, 22.6) | 4.5 × 10⁻⁹ |

Discovery: validation set (n = 59)

| Likely to benefit | 28/55 | 54.8 (16.3, NR) | Reference | |
|---|---|---|---|---|
| Unlikely to benefit | 4/4 | 5.8 (2.9, NR) | 3.9 (1.4, 11.4) | 0.012 |

Discovery: test set (n = 64)

| Likely to benefit | 33/57 | 30.2 (16.4, NR) | Reference | |
|---|---|---|---|---|
| Unlikely to benefit | 6/7 | 6.0 (2.4, NR) | 2.7 (1.1, 6.6) | 0.026 |

Discovery: validation + test set (n = 123)

| Likely to benefit | 61/112 | 40.6 (24.8, NR) | Reference | |
|---|---|---|---|---|
| Unlikely to benefit | 10/11 | 6.0 (3.3, NR) | 3.2 (1.6, 6.3) | 8.2 × 10⁻⁴ |

Independent validation cohort (n = 27)

| Likely to benefit | 6/23 | NR (15.8, NR) | Reference | |
|---|---|---|---|---|
| Unlikely to benefit | 3/4 | 6.0 (2.4, NR) | 5.6 (1.2, 25.5) | 0.027 |

The statistical significance was estimated in univariate Cox regression analyses of all demographic and clinical variables with and without adjustment. This was then used for variable selection and adjustment in the multivariable Cox regression analysis. Age, BRAF status, categorical LDH group, ECOG performance status, M stage, melanoma subtype, and line of therapy all achieved P<0.15 in both analyses (Table 27). Multivariable Cox regression analysis was then performed using the classifier prediction group as the primary independent variable and adjusting for the previously listed variables (Table 26). When applied to the full cohort, this model predicted that the risk of death for a patient that is classified as unlikely to benefit from ICI therapy based on their glycoproteomic risk score is about 3.4 times higher than if they were classified as likely to benefit, adjusting for the demographic and clinical variables (95% CI: 1.9, 6.1). Interestingly, variables such as age, categorical LDH group, ECOG performance status, subtype, and line of therapy generated models that were significant (P<0.05) without the inclusion of the classifier prediction variable. However, when the classifier prediction variable was included, the only predictors that remain statistically significant were classifier prediction (P=6.3×10⁻⁵), melanoma subtype (P=5.1×10⁻⁴), and line of therapy (P=5.9×10⁻³) (Table 26). Using a likelihood ratio test to compare these two multivariable Cox models, there is sufficient evidence that the model that includes the classifier prediction provides a significantly better fit than the model without it (P=2.0×10⁻⁴).

TABLE 26
Performance of Cox regression-based classifier in the discovery cohort with
multivariable adjustments. Small N in certain variable subgroups merits
combining the validation and test sets for the purposes of this table.

| | | | | | Validation + test sets | |
|---|---|---|---|---|---|---|
| | Full cohort (n = 202) | | Training set (n = 79) | | (n = 123) | |
| Variable | HR (95% CI) | P-value | HR (95% CI) | P-value | HR (95% CI) | P-value |
| Classifier prediction | 3.355 | 6.3 × 10⁻⁵ | 5.453 | 4.3 × 10⁻³ | 2.128 | 0.073 |
| (unlikely to benefit) | (1.854, 6.069) | | (1.704, 17.449) | | (0.931, 4.861) | |
| Age (continuous | 1.017 | 0.081 | 1.031 | 0.066 | 1.007 | 0.567 |
| years) | (0.998, 1.035) | | (0.998, 1.065) | | (0.983, 1.031) | |
| Positive BRAF | 1.197 | 0.455 | 1.187 | 0.704 | 1.266 | 0.439 |
| status (ref: | (0.747, 1.917) | | (0.490, 2.875) | | (0.696, 2.302) | |
| negative) | | | | | | |
| LDH category | | 0.328 | | 0.009 | | 0.396 |
| <ULN | Reference | | | | | |
| 1-2 × ULN | 1.073 | 0.762 | 0.955 | 0.916 | 0.994 | 0.983 |
| | (0.682, 1.688) | | (0.405, 2.254) | | (0.555, 1.779) | |
| >2 × ULN | 1.715 | 0.082 | 4.151 | 0.031 | 1.604 | 0.232 |
| | (0.934, 3.15) | | (1.143, 15.073) | | (0.739, 3.479) | |
| ECOG performance | | 0.222 | | 0.236 | | 0.007 |
| status | | | | | | |
| 0 | Reference | | | | | |
| 1 | 1.375 | 0.147 | 1.564 | 0.232 | 1.478 | 0.176 |
| | (0.894, 2.115) | | (0.751, 3.256) | | (0.839, 2.605) | |
| ≥2 | 1.941 | 0.084 | 0.374 | 0.282 | 5.511 | 1.2 × 10⁻⁴ |
| | (0.914, 4.121) | | (0.062, 2.245) | | (2.314, 13.127) | |
| M1 stage (ref: M0) | 2.146 | 0.109 | 1.744 | 0.429 | 4.932 | 0.033 |
| | (0.843, 5.464) | | (0.439, 6.928) | | (1.138, 21.375) | |
| Non-cutaneous | 2.00 | 5.1 × 10⁻⁴ | 3.026 | 3.5 × 10⁻³ | 1.528 | 0.118 |
| subtype (ref: | (1.353, 2.957) | | (1.44, 6.358) | | (0.898, 2.599) | |
| cutaneous) | | | | | | |
| Not first-line therapy | 1.841 | 5.9 × 10⁻³ | 1.981 | 0.112 | 2.073 | 7.8 × 10⁻³ |
| (ref: first-line) | (1.192, 2.842) | | (0.853, 4.603) | | (1.212, 3.548) | |

TABLE 27
Univariate Cox regression with respect to OS without and with adjustment
for classifier prediction (likely/unlikely to benefit) in full discovery
cohort; variables significant at P < 0.15 in both frameworks (marked
with an asterisk) are included in multivariable modeling shown in Table
25. Global p-values for categorical variables take missing values into account.

| | Without adjustment | | With adjustment | |
|---|---|---|---|---|
| Variable | HR (95% CI) | P-value | HR (95% CI) | P-value |
| Age (continuous years)* | 1.018 (1.003, 1.033) | 0.017 | 1.015 (1.00, 1.03) | 0.057 |
| Female sex (ref: male) | 0.851 (0.574, 1.262) | 0.422 | 0.759 (0.509, 1.13) | 0.174 |
| Pembrolizumab | 0.89 (0.615, 1.289) | 0.539 | 0.935 (0.645, 1.355) | 0.722 |
| treatment (ref: ipi/nivo) | | | | |
| Positive BRAF status | 1.452 (0.957, 2.202) | 0.08 | 1.394 (0.918, 2.117) | 0.119 |
| (ref: negative)* | | | | |
| LDH category* | | 7.3 × 10⁻³ | | 0.101 |
| <ULN | Reference | | | |
| 1-2xULN | 1.417 (0.939, 2.138) | 0.097 | 1.382 (0.916, 2.086) | 0.123 |
| >2xULN | 2.711 (1.579, 4.656) | 3.0 × 10⁻⁴ | 2.007 (1.142, 3.53) | 0.016 |
| ECOG performance | | 2.2 × 10⁻⁴ | | 0.112 |
| status* | | | | |
| 0 | Reference | | | |
| 1 | 1.353 (0.905, 2.022) | 0.14 | 1.307 (0.874, 1.955) | 0.193 |
| ≥2 | 4.352 (2.305, 8.216) | 5.8 × 10⁻⁶ | 2.372 (1.167, 4.823) | 0.017 |
| M1 stage (ref: M0)* | 2.43 (0.99, 5.963) | 0.053 | 2.284 (0.930, 5.609) | 0.072 |
| Non-cutaneous | 2.149 (1.479, 3.121) | 5.9 × 10⁻⁵ | 2.099 (1.444, 3.052) | 1.0 × 10⁻⁴ |
| subtype (ref: | | | | |
| cutaneous)* | | | | |
| Not first-line therapy | 1.352 (0.906, 2.018) | 0.14 | 1.434 (0.958, 2.144) | 0.08 |
| (ref: first-line)* | | | | |

Next, the Cox-based classifier was applied based on 14 markers to an independent cohort of 27 patient samples. Remarkably, the model achieved comparable separability between the likely and unlikely to benefit classification groups relative to the discovery cohort (HR=5.6; P=0.027) (FIG. 47E; Table 25). Notably, all four patients classified as unlikely to benefit from ICI therapy recorded progressive disease as best overall response, and three died (75%) (Table 28). Of the 23 patients who were classified as likely to benefit, 6 died (26%). Although the cohort was small in sample size, these data indicate that the classifier demonstrates high sensitivity to detect non-responders (Table 25).

TABLE 28
Demographic and clinical covariates in the external validation cohort stratified
by classifier prediction. Counts are followed by the appropriate column-wise
percentage, while continuous variables are summarized by medians and either IQRs
or, for time to event variables, 95% confidence limits (NR = not reached).

| Variable | Full cohort | Likely to benefit | Unlikely to benefit |
|---|---|---|---|
| Sample size | 27 | 23 (85) | 4 (15) |
| Female sex | 8 (30) | 8 (35) | 0 (0) |
| Age, yrs. (continuous) | 71 (66, 81) | 70 (65.5, 81) | 74.5 (70.5, 79.5) |
| Current ICI treatment | | | |
| Pembrolizumab monotherapy | 23 (85) | 19 (83) | 4 (100) |
| Ipilimumab/nivolumab combination | 4 (15) | 4 (17) | 0 (0) |
| Survival-related events | | | |
| Progression (PFS) event | 10 (37) | 6 (26) | 4 (100) |
| Death (OS) event | 9 (33) | 6 (26) | 3 (75) |
| Time to event, mos. | | | |
| (continuous) | | | |
| Progression | NR (9.9, NR) | NR (15.8, NR) | 2.6 (1.4, NR) |
| Death | 18.6 (15.8, NR) | NR (15.8, NR) | 6.0 (2.4, NR) |
| Best overall response | | | |
| Complete response | 5 (19) | 5 (22) | 0 (0) |
| Partial response | 5 (19) | 5 (22) | 0 (0) |
| Stable disease | 6 (22) | 6 (26) | 0 (0) |
| Progressive disease | 9 (33) | 5 (22) | 4 (100) |
| Missing | 2 (7) | 2 (9) | 0 (0) |

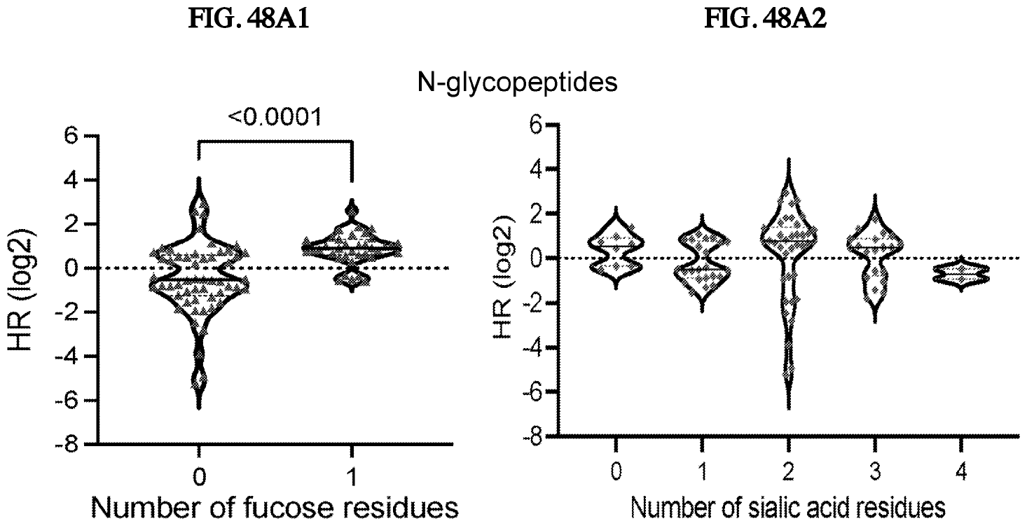

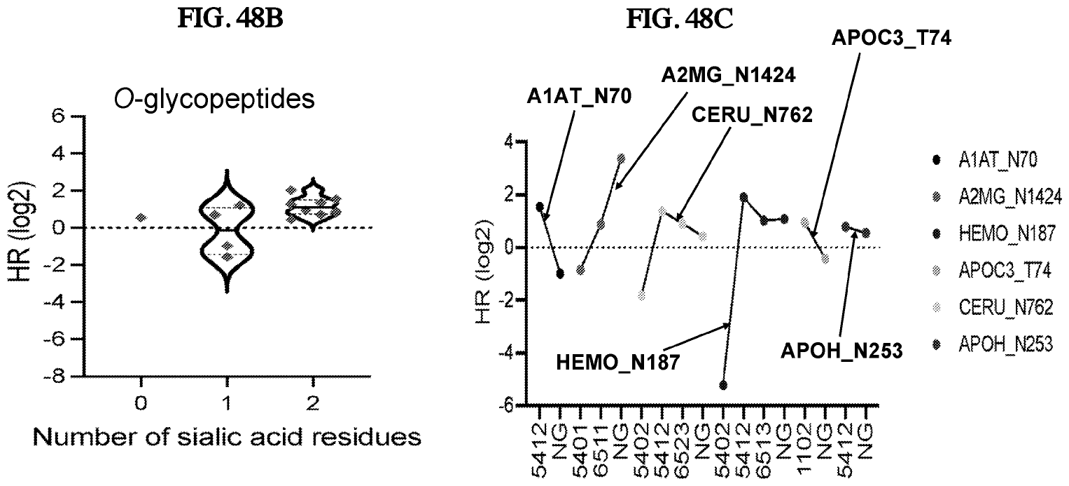

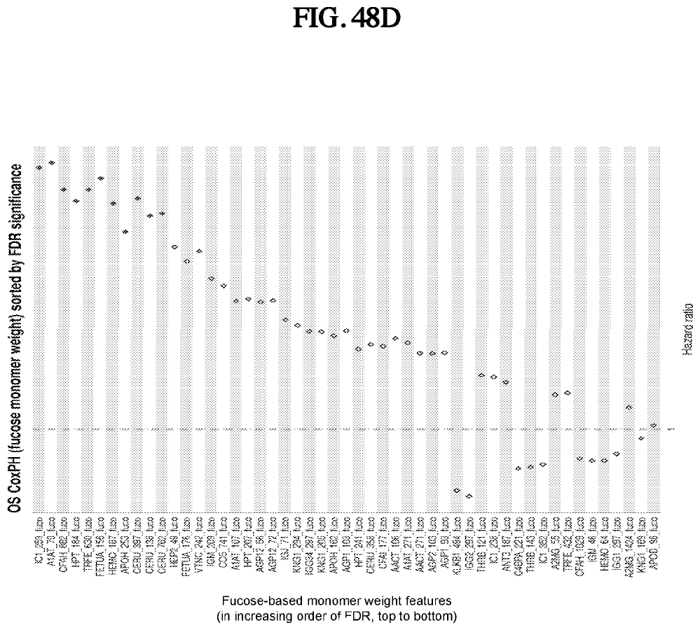

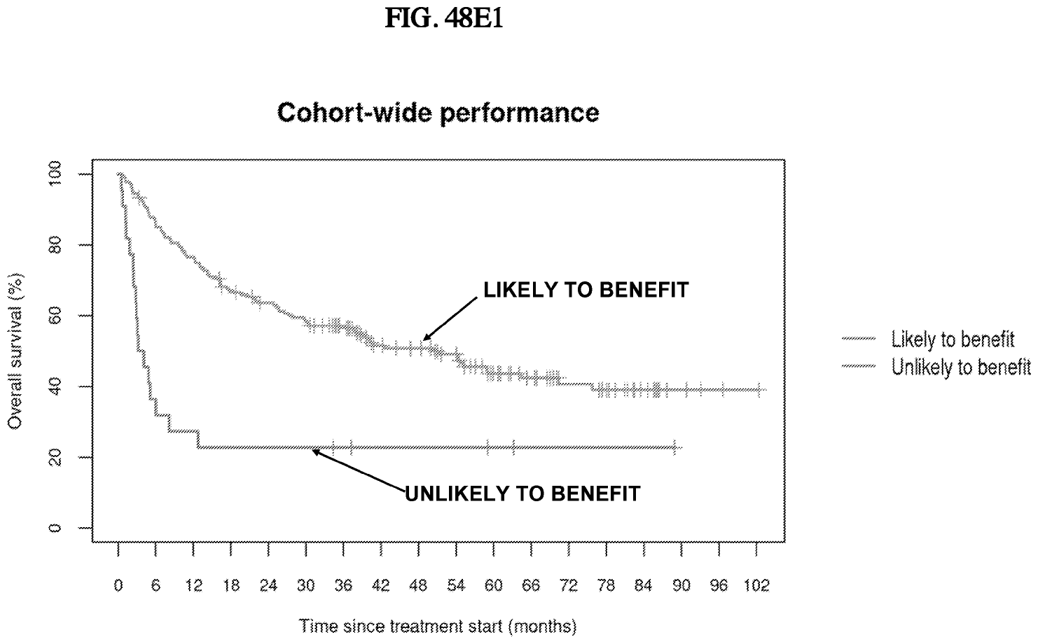

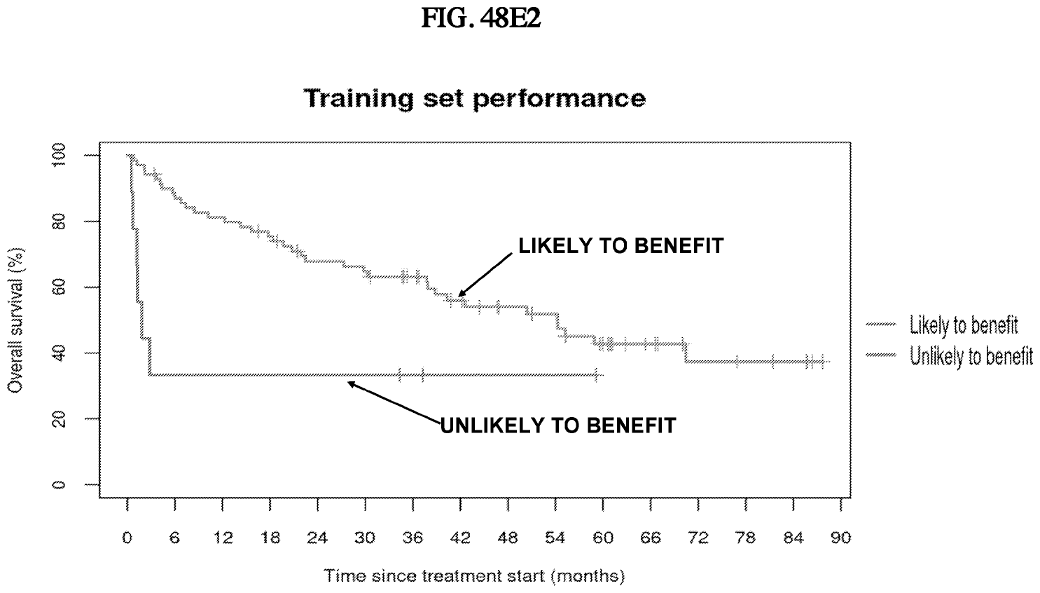

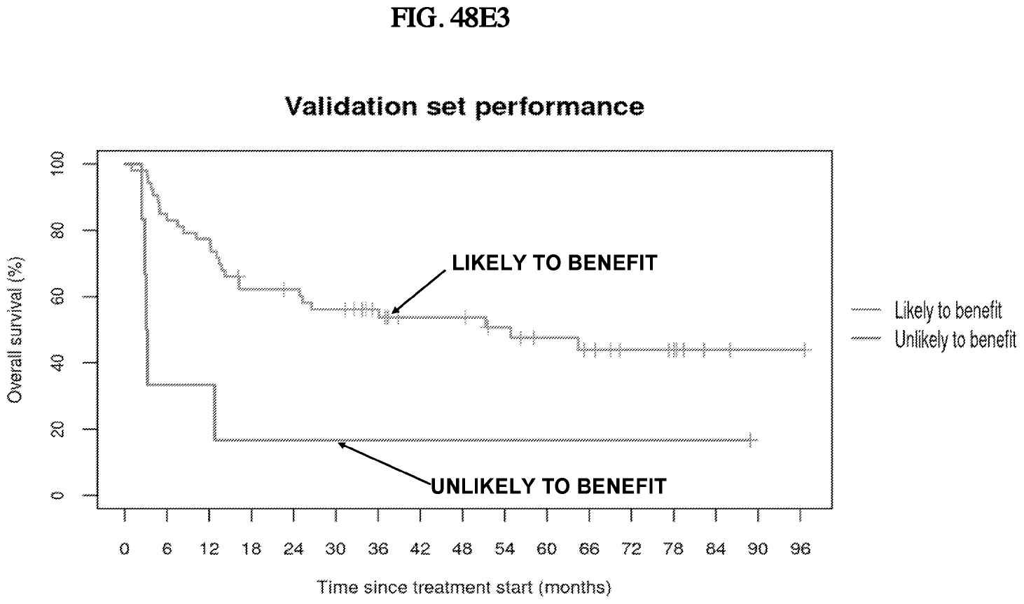

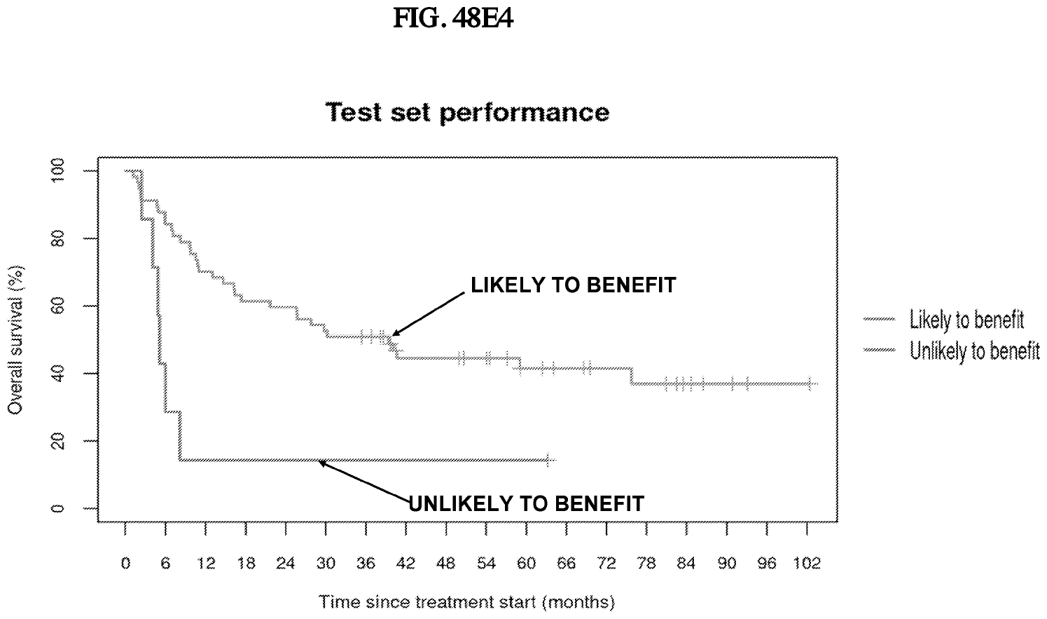

The fucosylation of N-glycopeptide biomarkers that are associated with reduced clinical benefit of ICI therapy was described. Biomarkers showing statistically significant differences in relative abundance in respect to OS (P<0.05) were selected for further structural analysis. Among the 114 markers, 91 were N-glycopeptides, all carrying complex-type glycans. Strikingly, when looking at fucose content, these markers separated in two distributions that reflected the benefit of treatment (FIG. 48A1), with fucosylated glycopeptides prevalent in patients with disease progression. Increased fucosylation has been described at the tumor site and has been linked to increased metastatic potential, modulation of TGF-beta signaling that is known to impact the distribution of T cells in the tumor, as well as PD-1 stability at the cell surface. Next, the sialic acid content in the same markers was analyzed, as alterations in sialic acid density in tumor cells have been extensively described in connection with inhibition of immune cell function. However, there was no correlation between the number of sialic acid units and benefit of ICI treatment (FIG. 48A2). Other mechanisms, including expression of the asialoglycoprotein receptor, might contribute to maintaining a homogeneous level of sialylation in circulating glycoproteins. Instead, a correlation was observed between the number of sialic units in O-linked glycopeptides and the reduced benefit of treatment (FIG. 48B); however, only a small number of O-glycopeptides were included in the panel. These species most likely correspond to disialylated core-1 O-glycan tetrasaccharides that contribute to binding of the immune-suppressive receptor Siglec-7 found on natural killer cells. Access to non-glycosylated forms of the peptides allowed us to also evaluate the relevance of site occupancy: as N-glycans are involved in protein folding and can prevent interactions with other proteins, changes in site occupancy may dramatically change protein activity. Interestingly, alpha1-antitrypsin (A1AT) and hemopexin exhibited opposite association with response to treatment in respect to site occupancy (FIG. 48C). Glycosylation of A1AT was described to affect binding to IL-8 and neutrophil activation.

To test the validity of this observation, site-specific glycosylation features that represent the average number of specific monosaccharides at a given site, weighted by glycopeptide occupancy. Of 51 fucose-dependent features across our full research assay, 11 were strongly associated with benefit from ICI therapy based on univariate age- and sex-adjusted Cox regression analysis (FIG. 48D). All 11 features were ultimately retained in a repeated cross-validated LASSO-regularized Cox regression model on a training set consisting of 40% of the cohort, yielding a hazard ratio of 2.9 (P=0.016) (FIG. 48E2). A validation set consisting of 30% of the cohort was used to tune model hyperparameters (FIG. 48E3). When applied to the remaining 30% of the cohort, this tuned model resulted in a HR of 3.5 (P=6.6×10⁻³) (FIG. 48E4). Altogether, these data support the notion that the fucosylation status of circulating glycoproteins is a critical parameter that stratifies patients based on the response to ICI therapy.

The group of site monomers in Tables 14 include site monomers that have been determined relevant to distinguishing at least between immunotherapy responsive melanoma and immunotherapy nonresponsive melanoma. For example, the group of site monomers may be used to predict the probability of having immunotherapy responsive melanoma for use in informing treatment decisions. In one or more embodiments, the at least 1 site monomer includes at least 1, at least 2, at least 3, at least 4, at least 5, or more of the site monomers listed in Table 29.

TABLE 29
Markers (Site Monomers) Associated with the Classification of Likely
to Benefit or Not Likely to Benefit from Immunotherapy for Melanoma

| | | | | Glycosylation | | |
|---|---|---|---|---|---|---|
| | | Protein | Peptide | Site within | | |
| Marker (Site | Protein | SEQ | SEQ | Prot SEQ | | |
| Monomer) | Name | ID NO | ID NO | ID NO | Monomer | Coeficient |
| IC1_253_fuco | Plasma | 556 | 2677 | 253 | Fucose | −0.004472 |
| | protease C1 | | | | | |
| | inhibitor | | | | | |
| A1AT_70_fuco | Alpha-1- | 657 | 2778 | 70 | Fucose | 0.2339732 |
| | antitrypsin | | | | | |
| CFAH_882_fuco | ComplementF | 253 | 2879 | 882 | Fucose | 0.1453662 |
| | actorH | | | | | |
| HPT_184_fuco | Haptoglobin | 354 | 2980 | 184 | Fucose | 0.2917658 |
| TRFE_630_fuco | Serotransferrin | 152 | 3081 | 630 | Fucose | 0.3406138 |
| FETUA_156_fuco | Alpha-2-HS- | 455 | 3182 | 156 | Fucose | −0.237215 |
| | glycoprotein | | | | | |
| HEMO_187_fuco | Hemopexin | 758 | 3283 | 187 | Fucose | −0.463297 |
| APOH_253_fuco | Beta-2- | 2576 | 3384 | 253 | Fucose | 0.0076569 |
| | glycoprotein1 | | | | | |
| CERU_397_fuco | Ceruloplasmin | 960 | 3485 | 397 | Fucose | 0.2983246 |
| CERU_138_fuco | Ceruloplasmin | 960 | 3586 | 138 | Fucose | 0.0751997 |
| CERU_762_fuco | Ceruloplasmin | 960 | 3687109 | 762 | Fucose | −0.146133 |

TABLE 30
Performance of monomer weight model using 11 fucose-specific
features derived from N-glycopeptides that achieved FDR
< 0.05 in age- and sex-adjusted Cox regression analysis

| | | | | |
|---|---|---|---|---|
| Classifier prediction | Events/N | Median OS (95% CI) | HR (95% CI) | P-value |

Full discovery cohort (n = 202)

| Likely to benefit | 96/180 | 50.4 (36.1, 75.7) | Reference | |
|---|---|---|---|---|
| Unlikely to benefit | 17/22 | 3.7 (2.8, 12.8) | 3.1 (1.9, 5.3) | 1.6 × 10⁻⁵ |

Discovery: training set (n = 79)

| Likely to benefit | 36/70 | 54.2 (37.9, NR) | Reference | |
|---|---|---|---|---|
| Unlikely to benefit | 6/9 | 1.8 (1.2, NR) | 2.9 (1.2, 7.0) | 0.016 |

Discovery: validation set (n = 59)

| Likely to benefit | 27/53 | 54.8 (24.8, NR) | Reference | |
|---|---|---|---|---|
| Unlikely to benefit | 5/6 | 3.2 (2.9, NR) | 3.8 (1.4, 10.0) | 6.7 × 10⁻³ |

Discovery: test set (n = 64)

| Likely to benefit | 33/57 | 39.4 (17.3, NR) | Refertableence | |
|---|---|---|---|---|
| Unlikely to benefit | 6/7 | 5.1 (4.1, NR) | 3.5 (1.4, 8.5) | 6.6 × 10⁻³ |

Although ICI therapies have revolutionized treatment of metastatic melanoma, only a subset of patients achieve a durable response. Therefore, it is important to develop assays that accurately identify patients unlikely to benefit from ICI therapy. Such efforts might also provide insight into specific factors that limit the efficacy of anti-PD-1, anti-PD-L1, and anti-CTLA-4 antibodies, and might inform selection of different or combination therapies. Here, pre-treatment liquid biopsy samples were analyzed using a novel platform and signatures were identified to predict response of metastatic melanoma. The data indicated that blood glycoprotein biomarker profiles accurately predict the likelihood of a beneficial or not beneficial response in melanoma patients.

Glycosylation is the most abundant and complex form of post-translational modification of proteins and it profoundly affects their structure, conformation, and function. The elucidation of the relevance of differential protein glycosylation as a novel class of biomarkers has so far been limited by the technical challenges of generating and interpreting this information at scale. Our platform helped uncover a specific fucosylation signature in plasma N-glycoproteins that tracks with the lack of response to ICI therapy. Fucosyltransferases, the enzymes responsible for addition of fucose to glycans, are overexpressed in malignant melanoma. Presence of fucose in the N-glycan of IgG1 significantly reduces its binding to FcgR. Core fucose has been shown to increase in metastatic melanoma cells and to affect cell invasion through the modulation of the stability of the adhesin L1CAM. Fucosylation can also alter TGF-β signaling and may therefore drive immune-excluded tumor phenotypes. Moreover, fucosylation has been directly linked to the stability of receptors such as cell surface PD-1. It is interesting that a similar fucosylation signature is observed both in the tumor microenvironment and in the periphery.

The role of A1AT glycosylation in binding to IL-8 and neutrophil activation has previously been described. Moreover, our functional analysis shows association of A1AT and AACT with platelet activation and thus a likely predisposition to hypercoagulable state, a process previously described to be associated with complications of advanced melanoma. Network analysis of cancer-causing genes relates the B2M gene product to melanoma. Beta2-microglobulin is found in association with the MHC class I alpha chain and is involved in antigen presentation. Beta2-microglobulin unbalance may promote tumor escape from recognition by CD8+ T cells, and may have a role in neutrophil degranulation. LRG1 has been described to modulate the tumor microenvironment on neutrophil activation. We propose that cells at the tumor site produce factors that are released in the periphery and modulate expression and glycosylation of protein in the liver and blood cells. The complex glycoproteomic response may in turn impact disease progression and response to treatment.

When key clinical variables are stratified by the classifier prediction in the discovery cohort, a few patterns become clear. For patients with higher LDH values at treatment start, the median OS decreases, irrespective of the classification as likely or unlikely to respond to ICI therapy. In other words, while belonging to a higher LDH category is associated with increased risk of death, samples classified as likely to benefit exhibit increased median OS compared to the group categorized as unlikely to benefit, regardless of the LDH category of a patient. The same pattern is observed with respect to BRAF mutation status: while being a BRAF mutant is perhaps slightly associated with increased risk of death, being classified as likely to benefit increases median OS relative to being classified as unlikely to benefit regardless of BRAF status. Somewhat in contrast, patients who have ECOG performance status above 1 demonstrate short median overall survival regardless of classification. However, samples classified as unlikely to benefit have comparable median overall survival regardless of ECOG performance status. This phenomenon is additional evidence that the glycoproteomic classifier provides significant utility in identifying patients who are unlikely to respond to ICI therapy, regardless of other physiological characteristics.

### Section 8—Predicting Peptide Retention Time in Mass Spectrometry

### A8. Description of Systems and Methods

The methods and systems disclosed herein may be used to predict retention times for peptides in liquid chromatography mass spectrometry (LC-MS) runs. In such runs, retention time (RT) is generally a measure of the time taken for an analyte (e.g., a peptide, a glycopeptide) to pass through a liquid chromatography column. It is calculated as the time from injection to detection of the analyte. The retention time can be used to determine a likely identity of the analyte, because different analytes can be expected to pass through at different rates based on their physicochemical properties. Although general estimates for retention times of peptides may be found in literature, retention times can vary based on actual run conditions (e.g., the instruments used, gas flow rate settings, column degradation, column length and other dimensions, temperature, reagents and other materials used, lab conditions). As such, methods and systems for more accurately predicting retention times for a particular LC-MS run is needed.

In some embodiments, the methods and systems disclosed herein may also be used for quality control purposes. Variations in retention time can also be the result of carryover from a previous run. By having a means for accurately predicting retention times based on actual run conditions, a user can calculate a difference between predicted retention times and measured retention times for known analytes. If the difference is greater than an acceptable threshold, the user may determine that there is unacceptable carryover from a previous run, and may thus be able to take remedial measures (e.g., by cleaning the liquid chromatography column and/or the mass spectrometer) to prevent inaccurate results.

Described herein are machine learning methods for accurately predicting retention times for peptides. Although the details of the systems and methods described here are optimized for peptides, the disclosure contemplates that similar approaches may be taken for glycopeptides (and/or glycoproteins, glycolipids, glycoRNA, glycoDNA, etc.) with sufficient optimization.

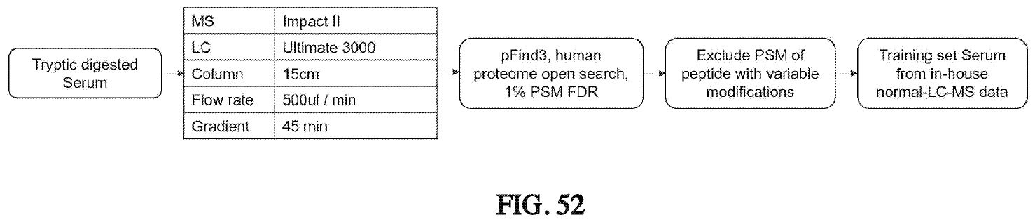

FIG. 52 illustrates an example workflow for generating training sets for training a machine learning model for predicting retention times for peptides.

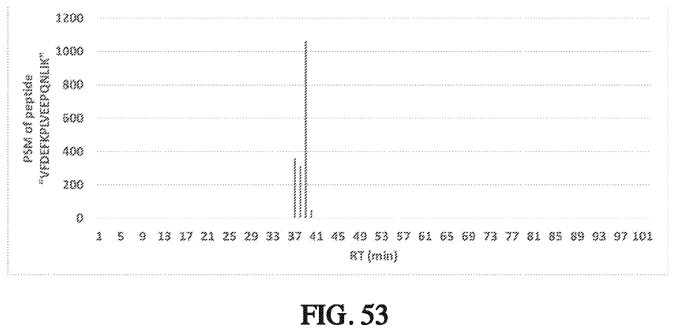

FIG. 53 illustrates the retention time distribution (n=1,783) of a particular peptide “VFDEFKPLVEEPQNLIK” from serum using the workflow illustrated in FIG. 52. The sample used to get this data before LC-MS steps was sourced from sigma serum which is a healthy human sample. This sample was then cleaved into peptides using tryptic serum digestion. The LC-model used was Thermo Ultimate 3000 and the column was a Waters HSS T3 with a size of 2.1×150 mm. The LC gradient length was 45 minutes. The mass spectrometer used was Bruker Impact II and the MS/MS acquisition was AutoMSMS CID.

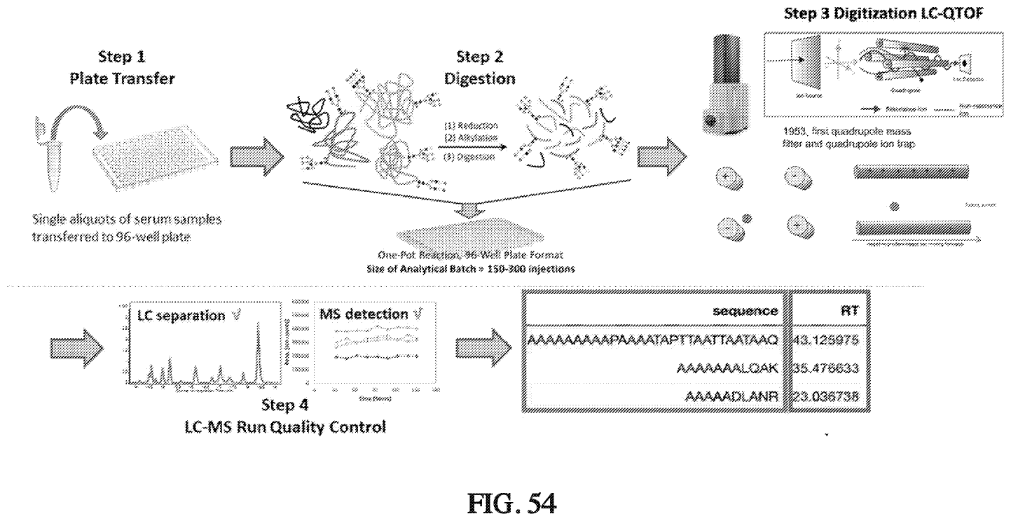

FIG. 54 illustrates an example LC-MS workflow and data extraction steps that may be employed. As shown, at step 1, serum samples are transferred to a container (e.g., 96-well plate). At step 2, the samples are digested so as to cleave the proteins within the samples into small peptides. At Step 3, these samples are passed through a liquid chromatography-mass spectrometer system and the peptides within the samples are detected. At Step 4, quality control is performed.

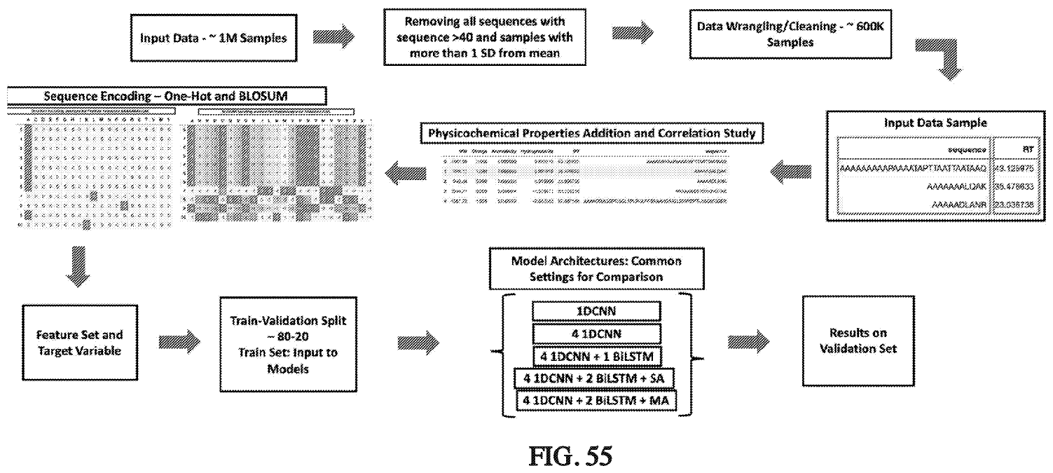

FIG. 55 illustrates an example workflow for predicting retention times based on human serum samples as described herein. In this example workflow, the input data included approximately 1 million samples of peptide sequences with their retention times. The number of sequences was then reduced by removing all sequences with greater than 40 amino acids and also by removing outliers for each peptide sequence by removing samples with more than 1 standard deviation from the mean (to avoid too much for the same peptide). These methods brought the sample size to ˜600,000 samples which was the input to the models described herein. This input included 8200 unique peptide sequences.

In some embodiments, the input features may include, in addition to the peptide sequences, PC properties that are associated with the peptides or peptide sequences. The physicochemical properties may be identified by a correlation study. This may ensure that redundant features are not included in the input feature set. For example, a Pearson correlation may be used on the entire set of identified physicochemical properties. Retention time may be included in this analysis, to observe dependency of each feature and the retention time. Another major aspect considered was how independent each feature was from the other. Any suitable PC properties may be accounted for. In particular, correlation studies performed on the sample sequences described herein indicated that the PC properties of molecular weight, charge, aromaticity, and hydrophobicity properties are significant and may be advantageous to include in the feature set.

In some embodiments, the peptide sequences may be encoded to allow for processing on neural networks using any suitable encoding process. For example, one-hot encoding or BLOSUM 62 methods may be used to encode the peptide sequences. In one-hot, a matrix with columns of 20 amino acids with one translational stop and rows with the length of a peptide sequence may be formed. To have a uniform input, post padding with 0 may be used on this input matrix to have a 40×21 matrix. This matrix is then compressed to a vector to form the peptide sequence embedding.

In BLOSUM 62, a similar approach may be used but instead the matrix columns may be 24 columns (20 columns for unique amino acids, 3 columns for special amino acid characters, and 1 column for a translation stop). This gives us an input matrix of 40×24 which is then compressed to a vector to form the peptide sequence embedding. The encoded sequence and its identified physicochemical features are then concatenated to give us the final feature set. Moreover, retention time is set as the target variable.

Finally, the feature set and the target variable may be normalized between 0 and 1. This is done to ensure it is easy for the model to understand the data and more importantly there is no bias towards bigger values in the input set. This set is then sent as input to the model for training and validation.

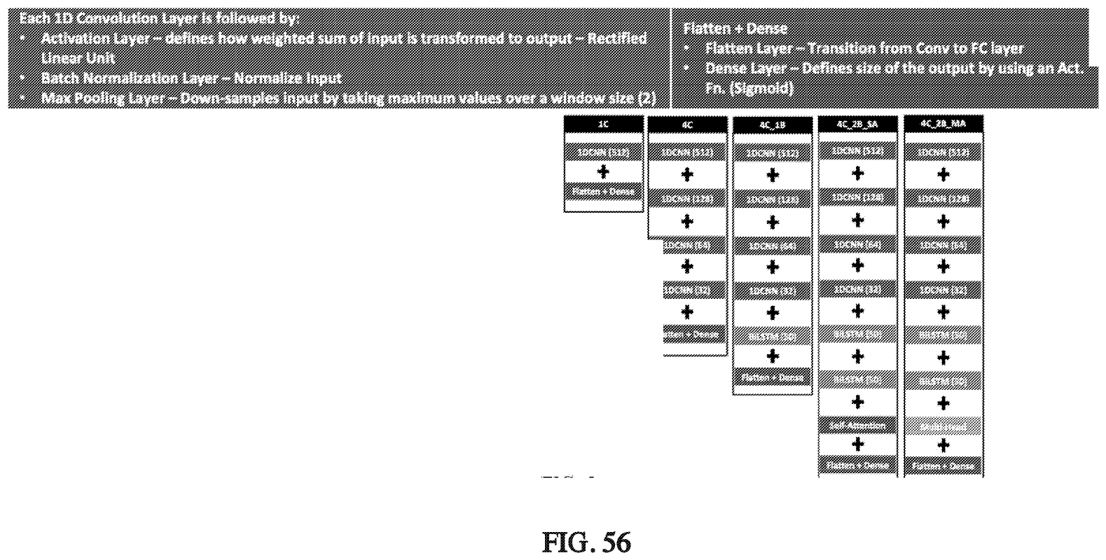

FIG. 56 illustrates a number of different architectures that were attempted for creating a model for predicting peptide retention times. For training, we first started with a base model which was chosen to be 1D CNN followed by an activation, batch normalization and max-pooling layer and this was then sent to the set of Flatten and Dense layers to get our output. We used an additive layer inclusion to observe differences in results from the previous model run. Next we added 3 more 1D CNN layers after the first one in the next architecture to see the significance of depth. Next, we added a BiLSTM layer to see the contribution due to the addition of an RNN layer in the architecture. Further, we added another BiLSTM layer followed by a Self-Attention layer and another variant with the addition of a Multi-Head Attention layer. These layers were again followed by the set of Flatten and Dense layers to give us the output.

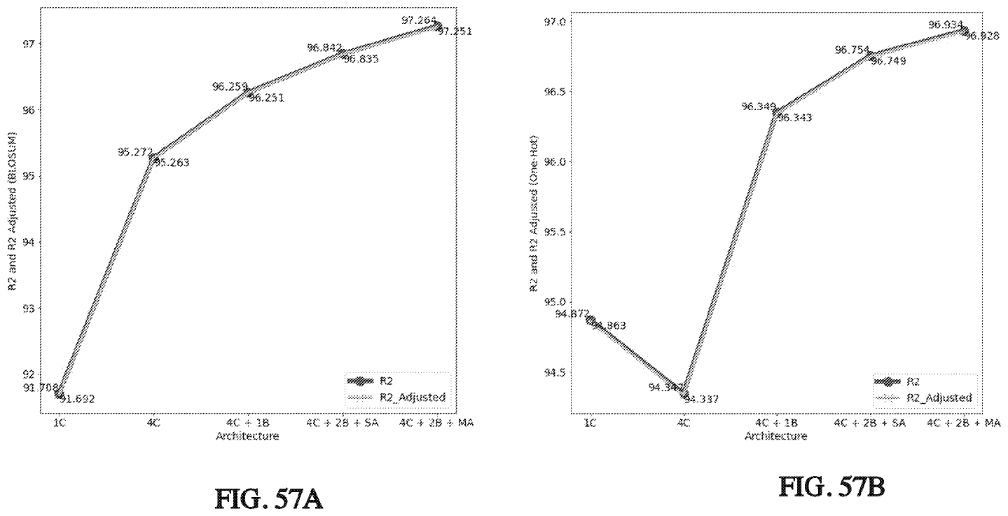

FIGS. 57A-57B illustrate plots of R2 and R2 Adjusted scores (BLOSUM and one-hot encodings) received using the various architectures noted in FIG. 56. For the base model we get the R2 and R2 Adjusted scores of approximately 92% for BLOSUM encoding. The Multi-Head Mechanism performed well for both encoding methods, achieving a R2 and R2-Adjusted value of approximately 97%. The fact that R2 and R2-Adjusted scores were close shows that the models are not overfitting. This is the network architecture labeled 4C_2B_MA in FIG. 56, which includes four 1DCNN layers, 2 BiLSTM layers, 1 Multi-Head Attention layer, and 1 Flatten+Dense layer.

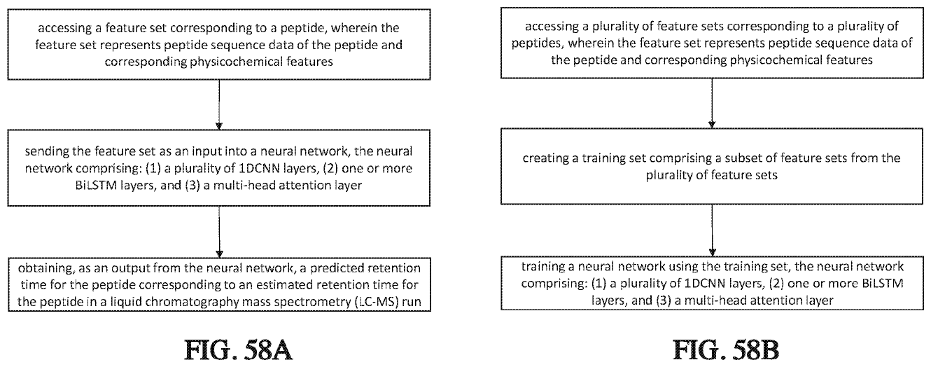

FIG. 58A illustrate an example method for predicting a retention time for a peptide. FIG. 58B illustrates an example method for training a neural network configured to predict retention times of peptides.

Particular embodiments may repeat one or more steps of the method of FIGS. 58A and 58B, where appropriate. Although this disclosure describes and illustrates particular steps of the method of FIGS. 58A and 58B as occurring in a particular order, this disclosure contemplates any suitable steps of the method of FIGS. 58A and 58B occurring in any suitable order. Furthermore, the disclosure contemplates that the methods may include additional steps that are not illustrated in FIGS. 58A and 58B. For example, they may include additional steps described elsewhere in this disclosure. Moreover, although this disclosure describes and illustrates particular components, devices, or systems carrying out particular steps of the method of FIGS. 58A and 58B, this disclosure contemplates any suitable combination of any suitable components, devices, or systems carrying out any suitable steps of the method of FIGS. 58A and 58B. Moreover, this disclosure contemplates that some or all of the computing operations described herein, including certain steps of the example method illustrated in FIGS. 58A and 58B, may be performed by circuitry of a computing device described herein, by a processor coupled to non-transitory computer readable storage media, or any suitable combination thereof.

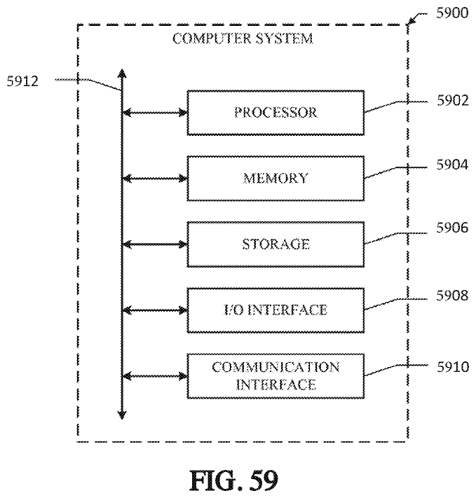

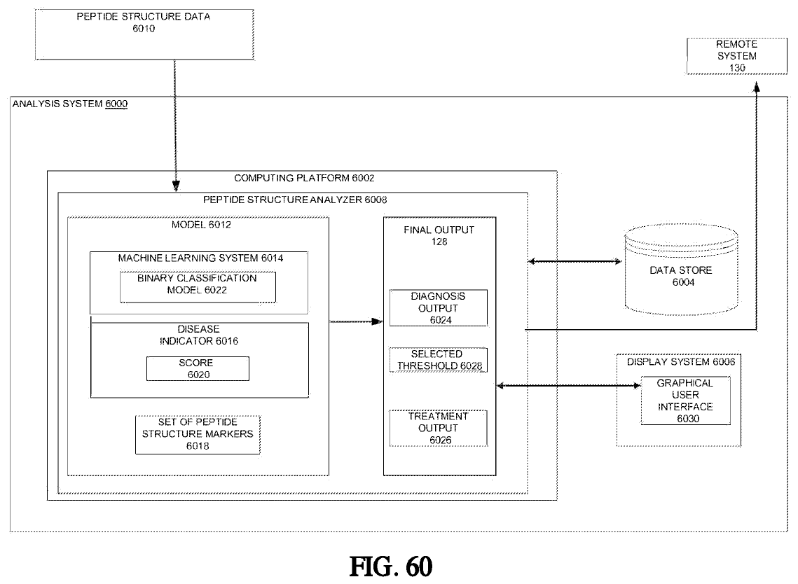

FIG. 59 illustrates an example computer system 5900. In particular embodiments, one or more computer systems 5900 perform one or more steps of one or more methods described or illustrated herein. In particular embodiments, one or more computer systems 5900 provide functionality described or illustrated herein. In particular embodiments, software running on one or more computer systems 5900 performs one or more steps of one or more methods described or illustrated herein or provides functionality described or illustrated herein. Particular embodiments include one or more portions of one or more computer systems 5900. Herein, reference to a computer system may encompass a computing device, and vice versa, where appropriate. Moreover, reference to a computer system may encompass one or more computer systems, where appropriate.

This disclosure contemplates any suitable number of computer systems 5900. This disclosure contemplates computer system 5900 taking any suitable physical form. As an example, computer system 5900 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 5900 may include one or more computer systems 5900; be unitary or distributed; span multiple locations; span multiple machines; span multiple data centers; or reside in a cloud, which may include one or more cloud components in one or more networks. Where appropriate, one or more computer systems 5900 may perform without substantial spatial or temporal limitation one or more steps of one or more methods described or illustrated herein. As an example, one or more computer systems 5900 may perform in real time or in batch mode one or more steps of one or more methods described or illustrated herein. One or more computer systems 5900 may perform at different times or at different locations one or more steps of one or more methods described or illustrated herein, where appropriate.

In particular embodiments, computer system 5900 includes a processor 5902, memory 5904, storage 5906, an input/output (I/O) interface 5908, a communication interface 5910, and a bus 5912. Although this disclosure describes and illustrates a particular computer system having a particular number of particular components in a particular arrangement, this disclosure contemplates any suitable computer system having any suitable number of any suitable components in any suitable arrangement.

In particular embodiments, processor 5902 includes hardware for executing instructions, such as those making up a computer program. As an example, to execute instructions, processor 5902 may retrieve (or fetch) the instructions from an internal register, an internal cache, memory 5904, or storage 5906; decode and execute them; and then write one or more results to an internal register, an internal cache, memory 5904, or storage 5906. In particular embodiments, processor 5902 may include one or more internal caches for data, instructions, or addresses. This disclosure contemplates processor 5902 including any suitable number of any suitable internal caches, where appropriate. As an example, processor 5902 may include one or more instruction caches, one or more data caches, and one or more translation lookaside buffers (TLBs). Instructions in the instruction caches may be copies of instructions in memory 5904 or storage 5906, and the instruction caches may speed up retrieval of those instructions by processor 5902. Data in the data caches may be copies of data in memory 5904 or storage 5906 for instructions executing at processor 5902 to operate on; the results of previous instructions executed at processor 5902 for access by subsequent instructions executing at processor 5902 or for writing to memory 5904 or storage 5906; or other suitable data. The data caches may speed up read or write operations by processor 5902. The TLBs may speed up virtual-address translation for processor 5902. In particular embodiments, processor 5902 may include one or more internal registers for data, instructions, or addresses. This disclosure contemplates processor 5902 including any suitable number of any suitable internal registers, where appropriate. Where appropriate, processor 5902 may include one or more arithmetic logic units (ALUs); be a multi-core processor; or include one or more processors 5902. Although this disclosure describes and illustrates a particular processor, this disclosure contemplates any suitable processor.

In particular embodiments, memory 5904 includes main memory for storing instructions for processor 5902 to execute or data for processor 5902 to operate on. As an example, computer system 5900 may load instructions from storage 5906 or another source (such as, for example, another computer system 5900) to memory 5904. Processor 5902 may then load the instructions from memory 5904 to an internal register or internal cache. To execute the instructions, processor 5902 may retrieve the instructions from the internal register or internal cache and decode them. During or after execution of the instructions, processor 5902 may write one or more results (which may be intermediate or final results) to the internal register or internal cache. Processor 5902 may then write one or more of those results to memory 5904. In particular embodiments, processor 5902 executes only instructions in one or more internal registers or internal caches or in memory 5904 (as opposed to storage 5906 or elsewhere) and operates only on data in one or more internal registers or internal caches or in memory 5904 (as opposed to storage 5906 or elsewhere). One or more memory buses (which may each include an address bus and a data bus) may couple processor 5902 to memory 5904. Bus 5912 may include one or more memory buses, as described below. In particular embodiments, one or more memory management units (MMUs) reside between processor 5902 and memory 5904 and facilitate accesses to memory 5904 requested by processor 5902. In particular embodiments, memory 5904 includes random access memory (RAM). This RAM may be volatile memory, where appropriate Where appropriate, this RAM may be dynamic RAM (DRAM) or static RAM (SRAM). Moreover, where appropriate, this RAM may be single-ported or multi-ported RAM. This disclosure contemplates any suitable RAM. Memory 5904 may include one or more memories 5904, where appropriate. Although this disclosure describes and illustrates particular memory, this disclosure contemplates any suitable memory.

In particular embodiments, storage 5906 includes mass storage for data or instructions. As an example, storage 5906 may include a hard disk drive (HDD), a floppy disk drive, flash memory, an optical disc, a magneto-optical disc, magnetic tape, or a Universal Serial Bus (USB) drive or a combination of two or more of these. Storage 5906 may include removable or non-removable (or fixed) media, where appropriate. Storage 5906 may be internal or external to computer system 5900, where appropriate. In particular embodiments, storage 5906 is non-volatile, solid-state memory. In particular embodiments, storage 5906 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 5906 taking any suitable physical form. Storage 5906 may include one or more storage control units facilitating communication between processor 5902 and storage 5906, where appropriate. Where appropriate, storage 5906 may include one or more storages 5906. Although this disclosure describes and illustrates particular storage, this disclosure contemplates any suitable storage.

In particular embodiments, I/O interface 5908 includes hardware, software, or both, providing one or more interfaces for communication between computer system 5900 and one or more I/O devices. Computer system 5900 may include one or more of these I/O devices, where appropriate. One or more of these I/O devices may enable communication between a person and computer system 5900. As an example, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 5908 for them. Where appropriate, I/O interface 5908 may include one or more device or software drivers enabling processor 5902 to drive one or more of these I/O devices. I/O interface 5908 may include one or more I/O interfaces 5908, where appropriate. Although this disclosure describes and illustrates a particular I/O interface, this disclosure contemplates any suitable I/O interface.

In particular embodiments, communication interface 5910 includes hardware, software, or both providing one or more interfaces for communication (such as, for example, packet-based communication) between computer system 5900 and one or more other computer systems 5900 or one or more networks. As an example, communication interface 5910 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 5910 for it. As an example, computer system 5900 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 5900 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 5900 may include any suitable communication interface 5910 for any of these networks, where appropriate. Communication interface 5910 may include one or more communication interfaces 5910, where appropriate. Although this disclosure describes and illustrates a particular communication interface, this disclosure contemplates any suitable communication interface.

In particular embodiments, bus 5912 includes hardware, software, or both coupling components of computer system 5900 to each other. As an example, bus 5912 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 5912 may include one or more buses 5912, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

The description herein is provided to enable one of ordinary skill in the art to make and use the invention and to incorporate it in the context of particular applications. Various modifications, as well as a variety of uses in different applications will be readily apparent to those skilled in the art, and the general principles defined herein may be applied to a wide range of embodiments. Thus, the inventions herein are not intended to be limited to the embodiments presented, but are to be accorded their widest scope consistent with the principles and novel features disclosed herein.

All the features disclosed in this specification, (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

### Additional Embodiments

The systems and methods of the present disclosure are further provided in the following non-limiting embodiments. In some embodiments, present systems and methods facilitate curation of a consensus list of identified glycomolecules from a plurality of glycomolecule search engines. In some embodiments, a system is a medical diagnostic or life sciences research method to identify the glycomolecule in a sample. In some embodiments, the method includes 5, 10, 15, 20, 25, 30, 40, 50 or more steps, or a number of steps in a range defined by any two of the preceding numbers. In some embodiments, at least 2, 3, 4, 5, 6, 7, 8, 9, 10, 15, 20, 25, 30, 40, 50 or more steps, or a number of steps in a range defined by any two of the preceding numbers, of the method are designed to be performed in sequence. In some embodiments, at least 2, 3, 4, 5, 6, 7, 8, 9, 10, 15, 20, 25, 30, 40, 50 or more steps, or a number of steps in a range defined by any two of the preceding numbers, of the method are designed to be performed in parallel.

In some embodiments, a system of the present disclosure is configured to provide notifications to the user of particular tasks that are time consuming or tasks that may create bottlenecks. In some embodiments, a system of the present disclosure is configured to suggest alternative tasks, or suggest abandoning a particular step. In some embodiments, a method of the present disclosure includes providing notifications to the user of particular tasks that are time consuming or tasks that may create bottlenecks. In some embodiments, a method of the present disclosure includes suggesting alternative tasks, or abandoning a particular step.

In any system or method of the present disclosure, in some embodiments, machine learning may be used for the predictions/notifications.

### Additional System Embodiments

In some embodiments, any of the systems of the present disclosure may be implemented in a cloud computing environment or cloud-based network. User interaction with the databases may be mediated via a central hub that stores and controls access to various interactions with the data. In some embodiments, the cloud computing environment may also provide sharing of protocols, analysis methods, libraries, as well as distributed processing for performing the analysis, and generating output reports. In some embodiments, the system may be implemented in a computer browser, on-demand or on-line.

In some embodiments, instructions or software written to perform the systems or methods as described herein is stored in some form of computer readable medium, such as memory (e.g., non-transitory memory), CD-ROM, DVD-ROM, memory stick, flash drive, hard drive, SSD hard drive, server, mainframe storage system and the like.

In some embodiments, systems and methods may be written in any of various suitable programming languages, for example compiled languages such as C, C#, C++, Fortran, and Java. Other programming languages could be script languages, such as Perl, MatLab, SAS, SPSS, Python, Ruby, Pascal, Delphi, R and PHP. In some embodiments, the system or method is written in C, C#, C++, Fortran, Java, Perl, R, or Python. In some embodiments, the system may include an independent application with data input and data display modules. Alternatively, the system may include a computer software product and may include classes wherein distributed objects comprise applications including computational methods as described herein.

An assay instrument, desktop computer, laptop computer, or server which may contain a processor in operational communication with accessible memory may comprise the instructions for implementation of the systems and/or methods of the present disclosure. In some embodiments, a desktop computer or a laptop computer is in operational communication with one or more computer readable storage media or devices and/or outputting devices. An assay instrument, desktop computer and a laptop computer may operate under a number of different computer-based operational languages, such as those utilized by Apple® based computer systems or PC based computer systems. An assay instrument, desktop and/or laptop computers and/or server system may further provide a computer interface for creating or modifying experimental definitions and/or conditions, viewing data results and monitoring experimental progress. In some embodiments, an outputting device may be a graphic user interface such as a computer monitor or a computer screen, a printer, a hand-held device such as a personal digital assistant (i.e., PDA, Blackberry, iPhone), a tablet computer (e.g., iPAD®), a hard drive, a server, a memory stick, a flash drive and the like.

A computer readable storage device or medium may be any device such as a server, a mainframe, a supercomputer, a magnetic tape system, and the like. In some embodiments, a storage device may be located onsite in a location proximate to the assay instrument, for example adjacent to or in close proximity to, an assay instrument. For example, a storage device may be located in the same room, in the same building, in an adjacent building, on the same floor in a building, on different floors in a building, etc., in relation to the assay instrument. In some embodiments, a storage device may be located off-site, or distal, to the assay instrument. For example, a storage device may be located in a different part of a city, in a different city, in a different state, in a different country, etc., relative to the assay instrument. In embodiments where a storage device is located distal to the assay instrument, communication between the assay instrument and one or more of a desktop, laptop, or server is typically via Internet connection, either wireless or by a network cable through an access point. In some embodiments, a storage device may be maintained and managed by the individual or entity directly associated with an assay instrument, whereas in other embodiments a storage device may be maintained and managed by a third party, typically at a distal location to the individual or entity associated with an assay instrument. In embodiments as described herein, an outputting device may be any device for visualizing data.

An assay instrument, desktop, laptop, and/or server system may be used itself to store and/or retrieve computer implemented software programs incorporating computer code for performing and implementing computational methods as described herein, data for use in the implementation of the computational methods, and the like. One or more of an assay instrument, desktop, laptop, and/or server may comprise one or more computer readable storage media for storing and/or retrieving software programs incorporating computer code for performing and implementing computational methods as described herein, data for use in the implementation of the computational methods, and the like. Computer readable storage media may include, but is not limited to, one or more of a hard drive, a SSD hard drive, a CD-ROM drive, a DVD-ROM drive, a floppy disk, a tape, a flash memory stick or card, and the like. Further, a network including the Internet may be the computer readable storage media. In some embodiments, computer readable storage media refers to computational resource storage accessible by a computer network via the Internet or a company network offered by a service provider rather than, for example, from a local desktop or laptop computer at a distal location to the assay instrument.

In some embodiments, computer readable storage media for storing and/or retrieving computer implemented software programs incorporating computer code for performing and implementing methods, e.g., computational methods, as described herein, data for use in the implementation of the methods, e.g., computational methods, and the like, is operated and maintained by a service provider in operational communication with an assay instrument, desktop, laptop and/or server system via an Internet connection or network connection.

In some embodiments, a hardware platform for providing a computational environment comprises a processor (i.e., CPU) wherein processor time and memory layout such as random access memory (i.e., RAM) are systems considerations. For example, smaller computer systems offer inexpensive, fast processors and large memory and storage capabilities. In some embodiments, graphics processing units (GPUs) can be used. In some embodiments, hardware platforms for performing computational methods as described herein comprise one or more computer systems with one or more processors. In some embodiments, smaller computers are clustered together to yield a supercomputer network.

In some embodiments, methods, e.g., computational methods, as described herein are carried out on a collection of inter- or intra-connected computer systems (i.e., grid technology) which may run a variety of operating systems in a coordinated manner. For example, the CONDOR framework (University of Wisconsin-Madison) and systems available through United Devices are exemplary of the coordination of multiple stand-alone computer systems for the purpose dealing with large amounts of data. These systems may offer Perl interfaces to submit, monitor and manage large sequence analysis jobs on a cluster in serial or parallel configurations.

### Examples

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1—Proteolytic Digestion and SDS PAGE Gel Electrophoresis

This example describes a method for improved proteolytic processing of a biological sample comprising a glycoprotein in preparation for mass spectrometry (MS) analysis, including but not limited to a targeted glycoproteomic analysis. Specifically, the method demonstrated herein comprises thermal denaturation in a thermocycler, and is compared to other conventional approaches. Poorly digested proteins (such as those resulting in missed cleavages) can lead to both system-based issues, such as clogged or partially clogged components, and analytic issues leading to poor signal, poor reproducibility, and poor quantification.

In the first step described for the method herein, ammonium bicarbonate (50 mM) and dithiothreitol (DTT) (50 mM) solutions were freshly prepared. The ammonium bicarbonate solution was used to make the DTT solution. Pre-thawed biological samples and controls were inspected for turbidity, hemolysis, clotting, and precipitation. Immediately prior to transfer, each biological sample and control was gently vortexed for 10 seconds. Using a single channel pipette, 5 μL of biological sample or control (e.g., plasma or serum) was transferred into a deep-well digestion plate, wherein the plate is compatible with thermal cycling. To this, the 35 μL of 50 mM ammonium bicarbonate solution was added. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute.

A separate sample plate was incubated in a thermal cycler for 5 minutes, wherein the thermal cycler was set to 100° C. with a lid temperature of 105° C. All heated plates were allowed to cool to room temperature before removing from the respective heat source and spinning at 370×g for 1 minute. After the spin, the plate seals were removed.

After protein denaturation, all samples were reduced by adding 20 μL of the 50 mM DTT solution into each sample and control well. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute. Plates were then incubated in a 60° C. water bath for 50 minutes. Plates were then removed from the water bath and centrifuged at 4,800×g for 1 minute before removing the plate seals.

Prior to the completion of this reduction incubation, a fresh 90 mM iodoacetamide (IAA) solution was prepared, and the container with the IAA solution was covered in foil. When ready, samples were alkylated by adding 20 μL of the 90 mM IAA solution into each sample and control well. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute. Plates were then incubated in the dark at room temperature for 30 minutes. After the incubation, plate seals were removed and 10 μL of the 50 mM DTT solution was added to quench any remaining IAA in solution. The plates were then sealed with a foil heat seal using a plate sealer and vortexed at 1400 RPM for 1 minute on a microplate mixer. Plates were centrifuged at 370×g for 1 minute and the plate seals were removed.

Prior to the completion of this alkylation incubation, fresh protease solutions were prepared (trypsin or trypsin/LysC combination). For example, for the trypsin solution, trypsin powder is dissolved in the 50 mM ammonium bicarbonate solution for a final concentration of 0.167 μg/μL trypsin solution. To the quenched biological samples and controls, 60 μL of the 0.167 μg/μL trypsin solution was added to each well. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute. Plates were then incubated in a 37° C. water bath for 18 hours. Plates were then removed from the water bath and centrifuged at 4,800×g for 1 minute before removing the plate seals.

20 μL of freshly prepared 9% formic acid solution was added to each well containing the proteolytic digested samples to stop the enzyme reaction. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute.

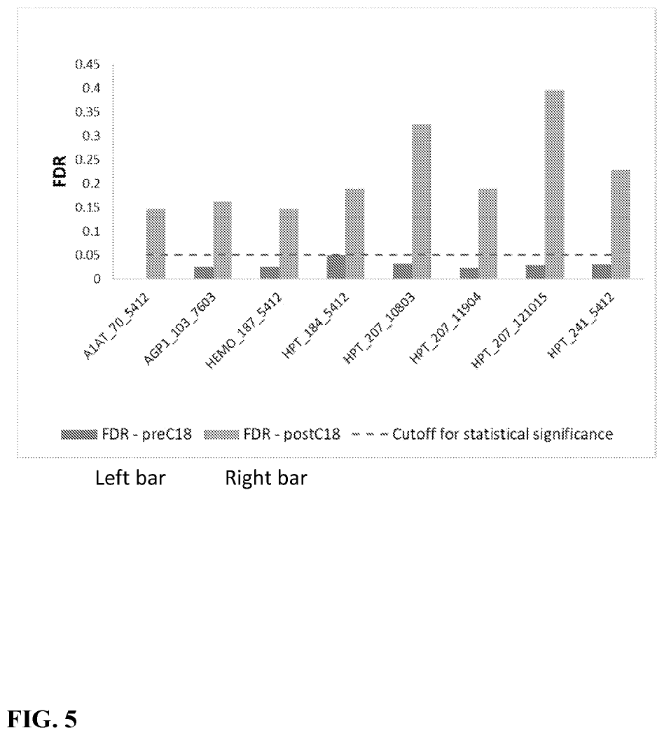

To compare the digestion efficiency based on the protein denaturation used during the sample preparation process, a relative concentration for each of the various peptide digests was used for performance of a SDS-PAGE gel analysis. Using the determined sample concentration, 20 μg of protein from each sample was transferred to a new tube and mixed with a gel loading dye and buffer (Laemmli sample buffer). Each sample was loaded onto a gel and run for 60 minutes at 150 V. Sample gels were stained in Coomassie stain and rinsed before imaging. As shown in FIG. 5A, thermal denaturation of serum at a 1:20 enzyme:sample weight ratio (denoted as the gel column Serum, TD, trypsin 1:20) significantly improved digestion efficiency of the biological sample, as determined by the decreased abundance of higher molecular weight species between 50 and 100 kDa compared to other conditions where the sample was not digested with enzyme (denoted as the gel column Serum, undigested), the sample was proteolytically digested as described above except that there was no thermal denaturation step (denoted as the gel column Serum, no TD, trypsin 1:20). For reference, the gel column denoted as Marker represents calibration standards that have a range of known molecular weight values. The gel column denoted as Serum, TD, trypsin 1:40 showed comparable digestion efficiency to the Serum, TD, trypsin 1:20, which suggests that the conditions of thermal denaturation of serum at 100° C. and trypsin to protein ratios ranging from 1:20 to 1:40 showed relatively high digestion efficiency. The gel column denoted as Serum, TD, trypsin+LysC 1:40 showed comparable digestion efficiency to the Serum, TD, trypsin 1:40, which suggests that the conditions of thermal denaturation of serum at 100° C. and either trypsin 1:40 or trypsin+LysC 1:40 both showed relatively similar and a high digestion efficiency.

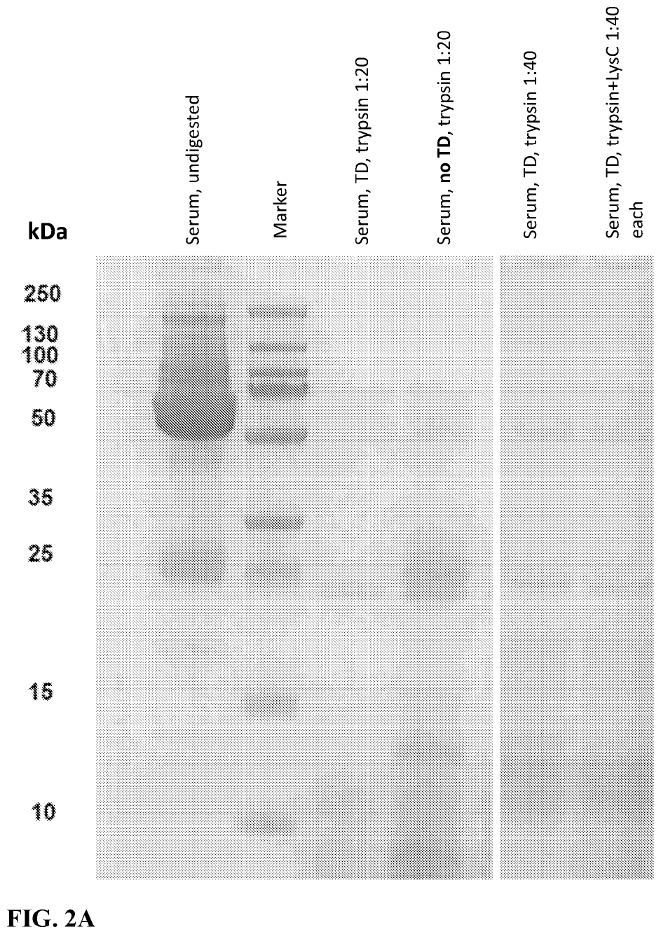

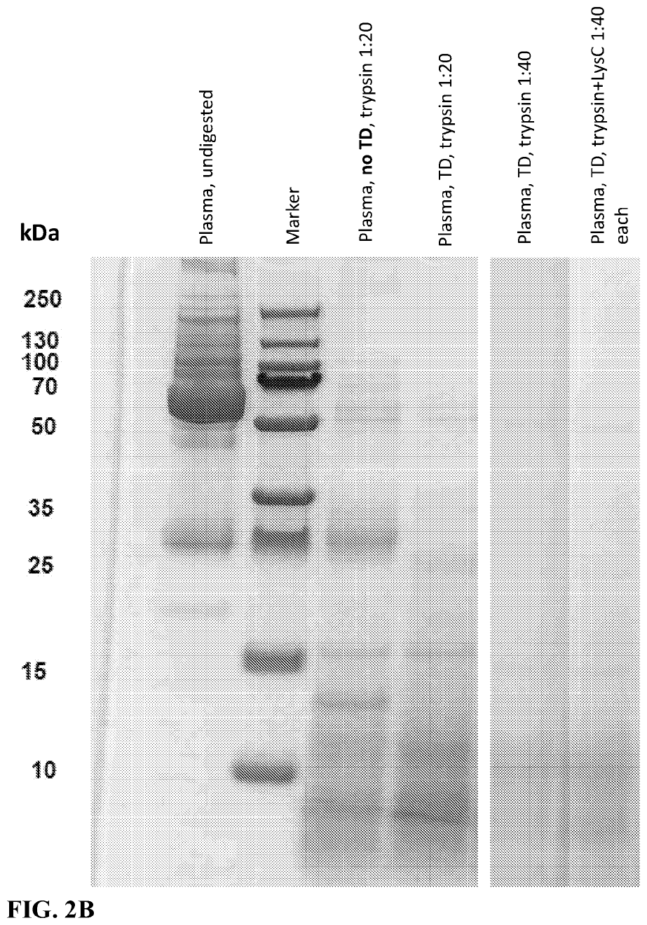

As shown in FIG. 2B, thermal denaturation of plasma at a 1:40 enzyme:sample weight ratio each for both trypsin and LysC (denoted as the gel column Plasma, TD, trypsin+LysC 1:40 each) significantly improved digestion efficiency of the biological sample, as determined by the decreased abundance of higher molecular weight species between 50 and 100 kDa compared to other conditions where the sample was not digested with enzyme (denoted as the gel column Plasma, undigested), the sample was proteolytically digested as described above except that there was no thermal denaturation step (denoted as the gel column Plasma, no TD, trypsin 1:20). For reference, the gel column denoted as Marker represents calibration standards that have a range of known molecular weight values. The gel column denoted as Plasma, TD, trypsin 1:40 showed comparable, but slightly better digestion efficiency to the Plasma, TD, trypsin 1:20, which suggests that the conditions of thermal denaturation of plasma at 100 C and trypsin to protein ratios ranging from 1:20 to 1:40 showed relatively similar digestion efficiency. The gel column denoted as “Plasma, TD, trypsin+LysC 1:40 each” showed comparable digestion efficiency to the Plasma, TD, trypsin 1:40, which suggests that the conditions of thermal denaturation of serum at 100 C and either trypsin 1:40 or trypsin+LysC 1:40 both showed relatively similar and a high digestion efficiency.

### Example 2—Mass Spectrometry of Proteolytic Digests with and without a C18 Clean-Up Step

This example demonstrates a method for mass spectrometry analysis using a liquid chromatography diversion technique to remove unwanted reagents from a sample containing proteolytic glycopeptides without the need for performing separate desalting steps.

Once samples were spun, they were either subjected to a clean-up using C18 cartridges on a liquid-handling system and then injected into an LC-MS system, or directly introduced into an LC-MS system using an LC diversion technique. For the C18 clean-up, the sample was diluted with sample buffer so that the sample had a concentration of 0.5% trifluoroacetic acid (TFA). Next, the sample diluted with sample buffer was flowed through a C18 cartridge (AssayMap C18 cartridge, 5 μL, from Agilent). The C18 cartridge was washed with a wash buffer containing 0.5% TFA to remove salt from the sample. An elution buffer of 0.1% TFA and 80% ACN was used to elute the sample from the C18 cartridge and then the sample was dried to remove the elution buffer. The dried sample was reconstituted in a solution of 0.1% formic acid in water before injecting into a LC-MS system.

All samples were subjected to the same instrument method, wherein 0-3 minutes were diverted to waste, 3-47.8 minutes were passed to the MS instrument, and 47.8-49 minutes were again diverted to waste. During this time course, a constant solvent gradient was completed. The aqueous mobile phase A was 0.1% formic acid in water (vol:vol), and the organic mobile phase B was 0.1% formic acid in acetonitrile (vol:vol). Separation of peptides and glycopeptides was performed using a binary gradient of 0.0-9.0 min, 1-10% B; 9.0-36.0 min, 10-25% B; 36.0-48.0 min, 25-44% B; 48.0-48.1 min, 44-1% B; 48.1-49.0 min, 1% B. The liquid chromatography system was an Agilent 1290 Infinity II UHPLC system that used a 20 μL loop volume, 4 μL injection volume, Waters ACQUITY UPLC Peptide HSS T3 Column, 100 Å port volume, 1.8 μm particle size, 2.1 mm×150 mm (diameter×length) with HSS T3 guard column, 2.1 mm×5 mm. The output of the chromatography column was either outputted to a waste channel or to the mass spectrometer via an electrospray ionization unit using a microprocessor controlled valve depending on the time of the chromatography run (see Table 1).

TABLE 1
Chromatography control parameters.

| Time | Scan Type | Divert vale |
|---|---|---|
| 0-3 minutes | dMRM | To waste |
| 3-47.8 minutes | dMRM | To MS |
| 47.8-49 minutes | dMRM | To waste |

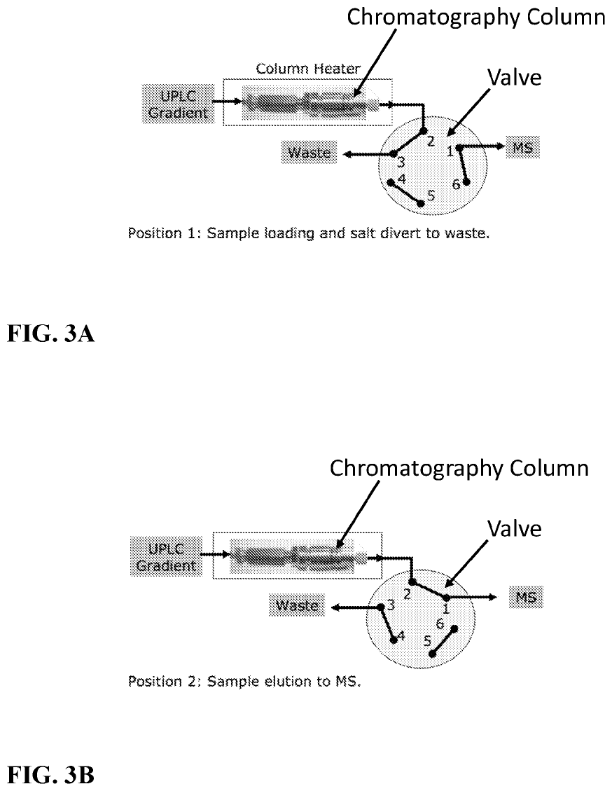

FIG. 3A shows a valve position 1 where the chromatography column output goes to waste via ports 2 and 3 of the diverter valve and FIG. 3B shows a valve position 2 where the chromatography output goes to waste via ports 2 and 3 of the diverter valve. The mass spectrometry system was an Agilent 6495C triple quadrupole mass spectrometer. Samples were introduced into the mass spectrometer using an electrospray ionization (ESI) source operated in the positive ion mode. Nitrogen drying and sheath gas temperatures were set at 290° C. and 300° C., respectively. Drying and sheath gas flow rates were set at 11 L/min and 12 L/min, respectively. The nebulizer pressure was set to 30 psi. Data acquired from the UHPLC/QqQ-MS was collected using Agilent MassHunter Workstation LC/MS Data Acquisition B10.1.67. Sample analysis was performed using a dynamic multiple reaction monitoring (dMRM) method. Collision induced dissociation was used for fragmentation.

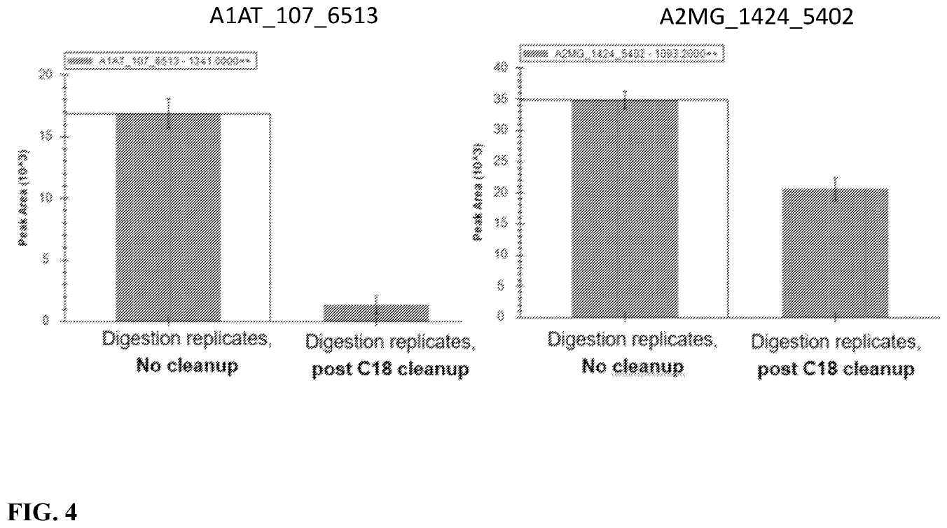

Results of the peptide fragment quantification were obtained with proteolytic digests that did not use a conventional C18 clean up step and instead used a post chromatography column valve with a salt diversion step and also with a more conventional approach where the proteolytic digest was de-salted with a C18 sample phase extraction material. Omission of the C18 clean-up step significantly enhanced the peptide fragment signal for multiple targets, as illustrated by FIG. 4 for two particular glycopeptides. During HPLC measurements using the conditions of this example, the glycopeptides A1AT_107_6513 and A2MG_1424_5402 had a relatively long retention time of 43 minutes and 44 minutes respectively indicating that both glycopeptides had a relatively high hydrophobicity. Details pertaining to A1AT_107_6513 and A2MG_1424_5402 are provided in Tables 2-4. Importantly, this signal enhancement was observed from both particularly substantially glycopeptides as well as substantially hydrophobic glycopeptides, indicating that standard C18 chromatography results in loss of glycopeptides on both ends of the hydrophobicity spectrum. It is worthwhile to note that although A1AT_107_6513 has 3 sialic acid groups and A2MG_1424_5402 has 2 sialic acid groups, these two glycopeptides are believed to have a long and hydrophobic peptide backbone. Both A1AT_107_6513 and A2MG_1424_5402 appeared to strongly bind to the C18 sample phase extraction material such that a significant portion of these glycopeptides were not eluted off the C18 sample phase extraction material and thus not measured in the mass spectrometric analysis. FIG. 4 illustrates that the MS signal measured with the diverter valve and without using the C18 sample phase extraction material provided a higher peak area for both A1AT_107_6513 and A2MG_1424_5402 providing an improved method of measuring hydrophobic glycopeptides without using a C18 sample phase extraction material.

The false discovery rate (FDR) was also assessed for glycopeptides identified using these two approaches (with and without C18 clean-up). Omission of the C18 clean-up step (labeled as “preC18”) significantly reduced the FDR in differentiating cases versus controls for all of targets (FIG. 5), suggesting omission of the clean-up step, and instead performing a diversion step, would be beneficial for identifying glycopeptides. It is possible that the omission of the C18 clean-up step and the use of the diversion step reduced the variance of the measured signal and/or increased the overall magnitude of the measured signal for the various transitions. Details pertaining to glycopeptides in FIG. 5 are provided in Tables 2-4.

TABLE 2
Glycoprotein information and sequences.

| SEQ | | | | |
|---|---|---|---|---|
| ID | Protein | | Uniprot | |
| NO | Abbreviation | Protein Name | ID | Protein Sequence |
| SEQ | A1AT | Alpha-1- | P01009 | MPSSVSWGILLLAGLCCLVPVSLAEDPQG |
| ID | | antitrypsin | | DAAQKTDTSHHDQDHPTFNKITPNLAEFA |
| NO: | | | | FSLYRQLAHQSNSTNIFFSPVSIATAFAMLS |
| 1 | | | | LGTKADTHDEILEGLNFNLTEIPEAQIHEGF |
| | | | | QELLRTLNQPDSQLQLTTGNGLFLSEGLKL |
| | | | | VDKFLEDVKKLYHSEAFTVNFGDTEEAKK |
| | | | | QINDYVEKGTQGKIVDLVKELDRDTVFAL |
| | | | | VNYIFFKGKWERPFEVKDTEEEDFHVDQV |
| | | | | TTV |
| | | | | KVPMMKRLGMFNIQHCKKLSSWVLLMKY |
| | | | | LGNATAIFFLPDEGKLQHLENELTHDIITKF |
| | | | | LENEDRRSASLHLPKLSITGTYDLKSVLGQ |
| | | | | LGITKVFSNGADLSGVTEEAPLKLSKAVH |
| | | | | KAVLTIDEKGTEAAGAMFLEAIPMSIPPEV |
| | | | | KFNKPFVFLMIEQNTKSPLFMGKVVNPTQ |
| | | | | K |
| SEQ | A2MG | Alpha-2- | P01023 | MGKNKLLHPSLVLLLLVLLPTDASVSGKP |
| ID | | macroglobulin | | QYMVLVPSLLHTETTEKGCVLLSYLNETV |
| NO: | | | | TVSASLESVRGNRSLFTDLEAENDVLHCV |
| 2 | | | | AFAVPKSSSNEEVMFLTVQVKGPTQEFKK |
| | | | | RTTVMVKNEDSLVFVQTDKSIYKPGQTVK |
| | | | | FRVVSMDENFHPLNELIPLVYIQDPKGNRI |
| | | | | AQWQSFQLEGGLKQFSFPLSSEPFQGSYKV |
| | | | | VVQKKSGGRTEHPFTVEEFVLPKFEVQVT |
| | | | | VPKIITILEEEMNVSVCGLYTYGKPVPGHV |
| | | | | TVSICRKYSDASDCHGEDSQAFCEKFSGQL |
| | | | | NSHGCFYQQVKTKVFQLKRKEYEMKLHT |
| | | | | EAQIQEEGTVVELTGRQSSEITRTITKLSFV |
| | | | | KVDSHFRQGIPFFGQVRLVDGKGVPIPNKV |
| | | | | IFIRGNEANYYSNATTDEHGLVQFSINTTN |
| | | | | VMGTSLTVRVNYKDRSPCYGYQWVSEEH |
| | | | | EEAHHTAYLVFSPSKSFVHLEPMSHELPCG |
| | | | | HTQTVQAHYILNGGTLLGLKKLSFYYLIM |
| | | | | AKGGIVRTGTHGLLVKQEDMKGHFSISIPV |
| | | | | KSDIAPVARLLIYAVLPTGDVIGDSAKYDV |
| | | | | ENCLANKVDLSFSPSQSLPASHAHLRVTA |
| | | | | APQSVCALRAVDQSVLLMKPDAELSASSV |
| | | | | YNLLPEKDLTGFPGPLNDQDNEDCINRHN |
| | | | | VYINGITYTPVSSTNEKDMYSFLEDMGLK |
| | | | | AFTNSKIRKPKMCPQLQQYEMHGPEGLRV |
| | | | | GFYESDVMGRGHARLVHVEEPHTETVRK |
| | | | | YFPETWIWDLVVVNSAGVAEVGVTVPDTI |
| | | | | TEWKAGAFCLSEDAGLGISSTASLRAFQPF |
| | | | | FVELTMPYSVIRGEAFTLKATVLNYLPKCI |
| | | | | RVSVQLEASPAFLAVPVEKEQAPHCICAN |
| | | | | GRQTVSWAVTPKSLGNVNFTVSAEALESQ |
| | | | | ELCGTEVPSVPEHGRKDTVIKPLLVEPEGL |
| | | | | EKETTFNSLLCPSGGEVSEELSLKLPPNVV |
| | | | | EESARASVSVLGDILGSAMQNTQNLLQMP |
| | | | | YGCGEQNMVLFAPNIYVLDYLNETQQLTP |
| | | | | EIKSKAIGYLNTGYQRQLNYKHYDGSYST |
| | | | | FGERYGRNQGNTWLTAFVLKTFAQARAYI |
| | | | | FIDEAHITQALIWLSQRQKDNGCFRSSGSL |
| | | | | LNNAIKGGVEDEVTLSAYITIALLEIPLTVT |
| | | | | HPVVRNALFCLESAWKTAQEGDHGSHVY |
| | | | | TKALLAYAFALAGNQDKRKEVLKSLNEE |
| | | | | AVKKDNSVHWERPQKPKAPVGHFYEPQA |
| | | | | PSAEVEMTSYVLLAYLTAQPAPTSEDLTSA |
| | | | | TNIVKWITKQQNAQGGFSSTQDTVVALHA |
| | | | | LSKYGAATFTRTGKAAQVTIQSSGTFSSKF |
| | | | | QVDNNNRLLLQQVSLPELPGEYSMKVTGE |
| | | | | GCVYLQTSLKYNILPEKEEFPFALGVQTLP |
| | | | | QTCDEPKAHTSFQISLSVSYTGSRSASNMA |
| | | | | IVDVKMVSGFIPLKPTVKMLERSNHVSRTE |
| | | | | VSSNHVLIYLDKVSNQTLSLFFTVLQDVPV |
| | | | | RDLKPAIVKVYDYYETDEFAIAEYNAPCS |
| | | | | KDLGNA |
| SEQ | AGP1 | Alpha-1-acid | P02763 | MALSWVLTVLSLLPLLEAQIPLCAN |
| ID | | glycoprotein 1 | | LVPVPITNATLDQITGKWFYIASAF |
| NO: | | | | RNEEYNKSVQEIQATFFYFTPNKTE |
| 3 | | | | DTIFLREYQTRQDQCIYNTTYLNVQ |
| | | | | RENGTISRYVGGQEHFAHLLILRDT |
| | | | | KTYMLAFDVNDEKNWGLSVYADK |
| | | | | PETTKEQLGEFYEALDCLRIPKSDV |
| | | | | VYTDWKKDKCEPLEKQHEKERKQ |
| | | | | EEGES |
| SEQ | HEMO | Hemopexin | P02790 | MARVLGAPVALGLWSLCWSLAIATPLPPT |
| ID | | | | SAHGNVAEGETKPDPDVTERCSDGWSFDA |
| NO: | | | | TT |
| 4 | | | | LDDNGTMLFFKGEFVWKSHKWDRELISER |
| | | | | WKNFPSPVDAAFRQGHNSVFLIKGDKVW |
| | | | | VYP |
| | | | | PEKKEKGYPKLLQDEFPGIPSPLDAAVECH |
| | | | | RGECQAEGVLFFQGDREWFWDLATGTMK |
| | | | | ER |
| | | | | SWPAVGNCSSALRWLGRYYCFQGNQFLR |
| | | | | FDPVRGEVPPRYPRDVRDYFMPCPGRGHG |
| | | | | HRN |
| | | | | GTGHGNSTHHGPEYMRCSPHLVLSALTSD |
| | | | | NHGATYAFSGTHYWRLDTSRDGWHSWPI |
| | | | | AHQ |
| | | | | WPQGPSAVDAAFSWEEKLYLVQGTQVYV |
| | | | | FLTKGGYTLVSGYPKRLEKEVGTPHGIILD |
| | | | | SV |
| | | | | DAAFICPGSSRLHIMAGRRLWWLDLKSGA |
| | | | | QATWTELPWPHEKVDGALCMEKSLGPNS |
| | | | | CSA |
| | | | | NGPGLYLIHGPNLYCYSDVEKLNAAKALP |
| | | | | QPQNVTSLLGCTH |
| SEQ | HPT | Haptoglobin | P00738 | MSALGAVIALLLWGQLFAVDSGN |
| ID | | | | DVTDIADDGCPKPPEIAHGYVEHSV |
| NO: | | | | RYQCKNYYKLRT |
| 5 | | | | EGDGVYTLNDKKQWINKAVGDKL |
| | | | | PECEADDGCPKPPEIAHGYVEHSVR |
| | | | | YQCKNYYKLRTE |
| | | | | GDGVYTLNNEKQWINKAVGDKLP |
| | | | | ECEAVCGKPKNPANPVQRILGGHL |
| | | | | DAKGSFPWQAKMV |
| | | | | SHHNLTTGATLINEQWLLTTAKNL |
| | | | | FLNHSENATAKDIAPTLTLYVGKK |
| | | | | QLVEIEKVVLHP |
| | | | | NYSQVDIGLIKLKQKVSVNERVMPI |
| | | | | CLPSKDYAEVGRVGYVSGWGRNA |
| | | | | NFKFTDHLKYVM |
| | | | | LPVADQDQCIRHYEGSTVPEKKTP |
| | | | | KSPVGVQPILNEHTFCAGMSKYQE |
| | | | | DTCYGDAGSAFA |
| | | | | VHDLEEDTWYATGILSFDKSCAVA |
| | | | | EYGVYVKVTSIQDWVQKTIAEN |

TABLE 3
Structures associated with FIGS. 4 and 5.

| | | | Linking | Linking | |
|---|---|---|---|---|---|
| | | | Site Pos. | Site Pos. | |
| SEQ | Peptide Structure (PS) | | in Protein | in Peptide | Glycan |
| ID NO | NAME | Peptide Sequence | Sequence | Sequence | Structure |
| SEQ ID | A1AT_70_5412 | QLAHQSNSTNI | 70 | 7 | 5412 |
| NO: 6 | | FFSPVSIATAFA | | | |
| | | MLSLGTK | | | |
| SEQ ID | AGP1_103_7603 | ENGTISR | 103 | 2 | 7603 |
| NO: 7 | | | | | |
| SEQ ID | HEMO_187_5412 | SWPAVGNCSS | 187 | 7 | 5412 |
| NO: 8 | | ALR | | | |
| SEQ ID | HPT_184_5412 | MVSHHNLTTG | 184 | 6 | 5412 |
| NO: 9 | | ATLINEQWLLT | | | |
| | | TAK | | | |
| SEQ ID | HPT_207_10803 | NLFLNHSENAT | 207 & 211 | 5 & 9 | 5401 & |
| NO: 10 | | AK | | | 5402 |
| SEQ ID | HPT_207_11904 | NLFLNHSENAT | 207 & 211 | 5 & 9 | 5402 & |
| NO: 11 | | AK | | | 6502 |
| SEQ ID | HPT_207_121015 | NLFLNHSENAT | 207 & 211 | 5 & 9 | 6502 & |
| NO: 12 | | AK | | | 6513 |
| SEQ ID | HPT_241_5412 | VVLHPNYSQV | 241 | 6 | 5412 |
| NO: 13 | | DIGLIK | | | |

TABLE 4
Glycan structures associated with FIGS. 4 and 5.

| Glycan Structure GL NO. | Structure | Composition |
|---|---|---|
| 5401 | 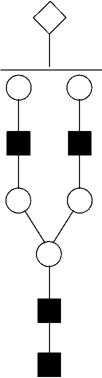 | Hex(5)HexNAc(4)Fuc(0)NeuAc(1) |
| 5402 | 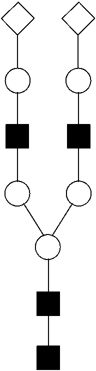 | Hex(5)HexNAc(4)Fuc(0)NeuAc(2) |
| 5412 | 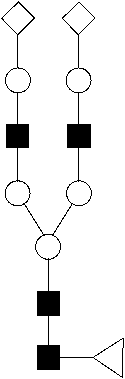 | Hex(5)HexNAc(4)Fuc(1)NeuAc(2) |
| 6502 | 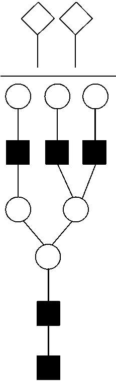 | Hex(6)HexNAc(5)Fuc(0)NeuAc(2) |
| 6513 | 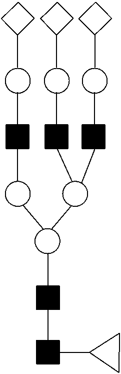 | Hex(6)HexNAc(5)Fuc(1)NeuAc(3) |
| 7603 | 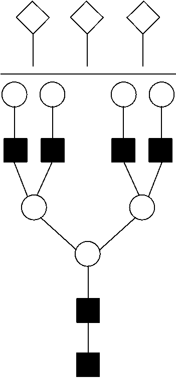 | Hex(7)HexNAc(6)Fuc(0)NeuAc(3) |

Legend for Table 4

[symbol]

Glc

[symbol]

Gal

[symbol]

Man

[symbol]

Fuc

[symbol]

NeuSAc

[symbol]

GlcNAc

[symbol]

GalNAc

[symbol]

ManNAc

### Example 3

This example demonstrates a study of the impact of trypsin amount on the resulting detected peak area for a glycopeptide.

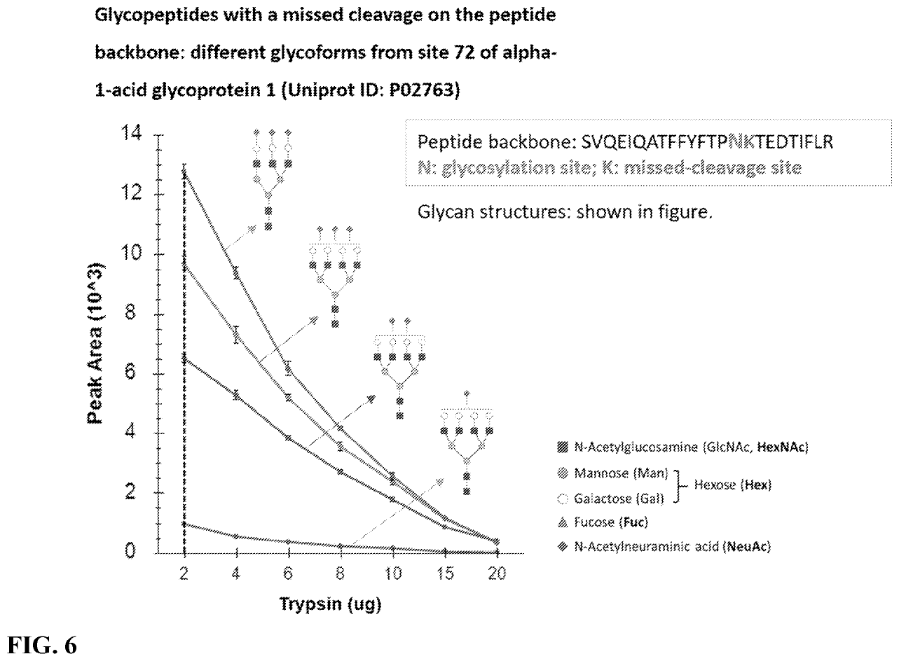

To compare the effects of increasing trypsin amounts, a serum sample was prepared as described in Example 1 and 2 with a titration of trypsin protease amounts, ranging from 2 μg to 40 μg, corresponding to an enzyme to protein weight ratio of 1:400 to 1:20, respectively. A targeted set of glycoforms (i.e., same parent peptide sequence with different glycosylation) and the expected peptide fragments were then quantified and analyzed to determine the optimal concentration for peak peptide digestion efficiency. In this example, a glycoform was chosen that had a missed trypsin cleavage. As shown in FIG. 6, different parent fragments have vastly different abundances in the presence of low amounts of trypsin protease. In the presence of higher trypsin concentrations, the parent peptides are digested to the expected peptide fragments and reduce the amount of missed cleavage for each parent peptide. The higher trypsin to protein weight ratio presumably helps overcome any steric effects caused by the glycans proximate to the cleavage site. This increase in digestion efficiency resulted in higher peptide fragment signal and increased reproducibility by reducing the amount of missed tryptic cleavages. However, at higher concentrations of trypsin, an increase in non-specific cleavage can be observed.

Thus, accounting for digestion efficiency and non-specific cleavage, this work demonstrated that an enzyme to protein ratio of about 1:40 maximized the MS signal for expected sample peptide fragments and helped reduce the amount of missed tryptic cleavages.

### Example 4

This example demonstrates context-specific use of one or more proteases and assessment for the improvement of sample signal reproducibility.

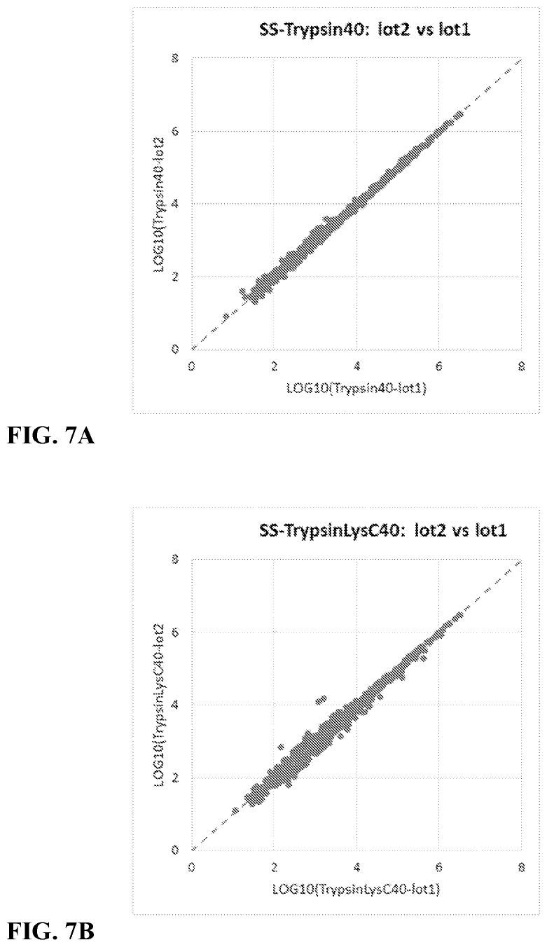

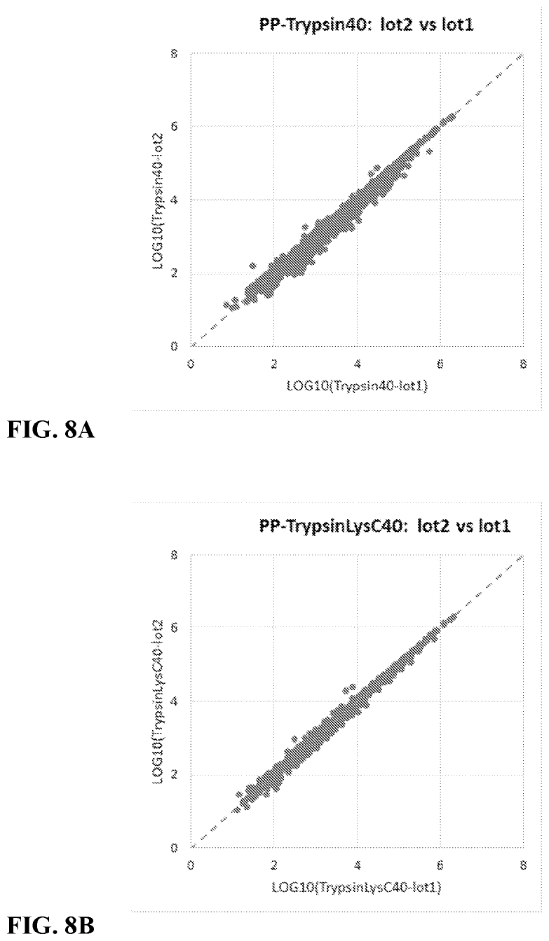

Serum sample digestion and plasma sample digestion was performed according to Examples 1 and 2 (using thermal denaturation and the diverter valve). Serum and plasma samples were digested with two lots of various proteases (Trypsin gold and Trypsin gold plus LysC) and a control protease reagent before being further processed and analyzed for sample reproducibility, as determined by the linear relationship. For serum samples, lot-to-lot reproducibility of trypsin alone was better than trypsin plus LysC, as shown in FIGS. 7A and 7B where each data point corresponds to a measured abundance value for either a peptide or glycopeptide generated with lot 1 and lot 2. The cloud of scatter points in FIG. 7A was tighter and closer to the unity line for the case where 2 lots of only methylated trypsin compared to the cloud of scatter points in FIG. 7B that was more diffuse from the unity line for the case where 2 lots of methylated trypsin plus LysC were tested for variability. For plasma samples, lot-to-lot reproducibility of trypsin plus LysC was better than trypsin alone, as shown in FIGS. 8A and 8B. The cloud of scatter points in FIG. 8A was more diffuse and away from the unity line for the case where 2 lots of only methylated trypsin compared to the cloud of scatter points in FIG. 8B that was more compact and closer to the unity line for the case where 2 lots of methylated trypsin plus LysC were tested for variability.

Thus, the use of trypsin plus LysC digestion demonstrated improved reproducibility for plasma sample analysis, but not serum sample analysis.

### Example 5

This example demonstrates a study of performing sample reduction with different DTT concentrations (10 mM or 20 mM), DTT incubation temperatures (37° C. or 60° C.), and DTT incubation duration (30 minutes or 50 minutes.

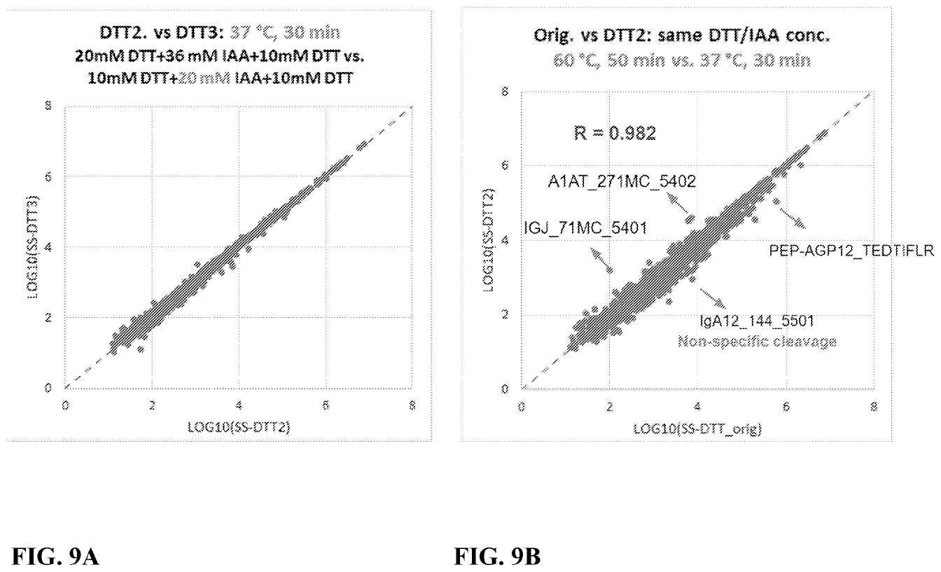

To compare the effects of varying reduction conditions, a serum sample was prepared as described in Example 1 and 2 with the reduction step containing two different reaction conditions. Standard DTT reduction practice entails incubation with 10 or 20 mM DTT at 37° C. for 30 minutes, and this was compared to 10 or 20 mM DTT at 60° C. for 50 minutes as illustrated by FIG. 9A. After sample reduction, the samples were processed and injected into the instrument for MS analysis. As shown in FIG. 9B, DTT reduction at higher temperatures and for longer times significantly reduced the amount of missed cleavage in the serum sample at both DTT concentrations. This is observed broadly across most of the quantified parent peptides and expected fragment peptides.

### Example 6

This Example demonstrates a study of sample signal when using formic acid to quench a protease, trypsin, after digestion.

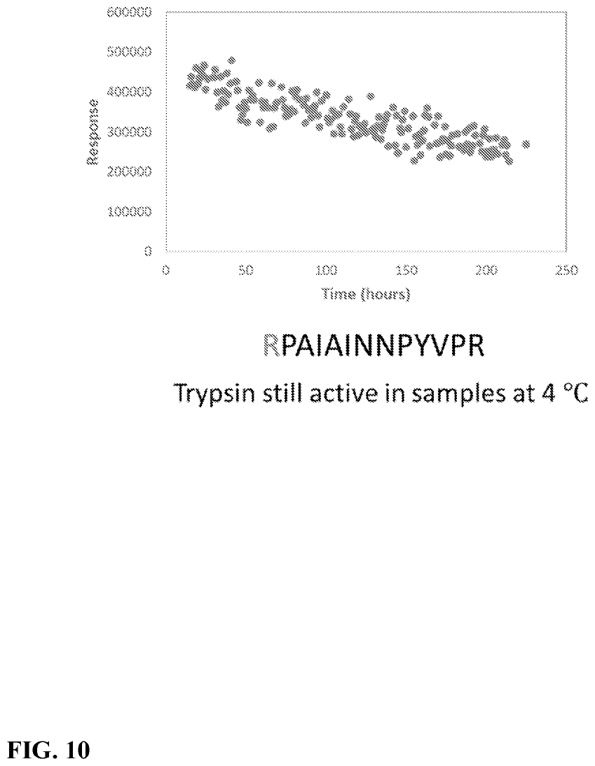

After sample digestion by trypsin protease, 20 μL of freshly prepared 9% formic acid solution was added to one of the sample wells. The other sample well was treated with a control buffer to compare the effects of trypsin quenching after sample digestion. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute. Each sample was further processed as described in Example 2 and directly injected on the UHPLC-QQQ instrument. To compare signal reproducibility of the two sample preparations, a particular peptide fragments RPAIAINNPYVPR was targeted for each sample and analyzed every hour for 10 days. As shown in FIG. 10, the peptide fragment signal obtained from the unquenched sample declined over the extended time course. This decline in signal is linear and significant effects are observed within the first 24 hours of analysis that was caused by the non-specific cleavage that occurred at the first R group adjacent to the proline residue. The quenched sample did not display this decline and the peptide fragment signal remains constant during the 10 day time course.

Thus, the complete protease inactivation by formic acid quenching demonstrates improved sample integrity for robust and reproducible signal analysis by reducing the amount of non-specific tryptic cleavages at a lysine or arginine that is adjacent to a proline.

### Example 7

This example demonstrates a method for processing a proteolytically digested sample to produce a processed sample suitable for use in a liquid chromatography-mass spectrometry (LC-MS) analysis, the method comprising subjecting a proteolytically digested sample to a solid phase extraction column comprising a C18 reversed-phase medium using a polypeptide loading amount relative to the binding capacity of the solid phase extraction column that results in improved peptide and glycopeptide analysis.

Human serum samples were subjected to a proteolytic digestion technique with trypsin. Briefly, each sample and control was gently vortexed for 10 seconds. Using a single channel pipette, 5 μL of serum sample or control was transferred into a deep-well digestion plate, wherein the plate is compatible with thermal cycling. To this, the 35 μL of 50 mM ammonium bicarbonate solution was added. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute. A separate sample plate was incubated in a thermal cycler for 5 minutes, wherein the thermal cycler was set to 100° C. with a lid temperature of 105° C. All heated plates were allowed to cool to room temperature before removing from the respective heat source and spinning at 370×g for 1 minute. After the spin, the plate seals were removed. After protein denaturation, all samples were reduced by adding 20 μL of the 50 mM DTT solution into each sample and control well. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute. Plates were then incubated in a 60° C. water bath for 50 minutes. Plates were then removed from the water bath and centrifuged at 4,800×g for 1 minute before removing the plate seals. Samples were alkylated by adding 20 μL of the 90 mM IAA solution into each sample and control well. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute. Plates were then incubated in the dark at room temperature for 30 minutes. After the incubation, plate seals were removed and 10 μL of the 50 mM DTT solution was added to quench any remaining IAA in solution. The plates were then sealed with a foil heat seal using a plate sealer and vortexed at 1400 RPM for 1 minute on a microplate mixer. Plates were centrifuged at 370×g for 1 minute and the plate seals were removed. A trypsin digestion was then performed followed by quenching of the protease via addition of an acid.

The resulting peptides were loaded onto AssayMAP C18 reversed-phase cartridges on an Agilent Bravo Platform for sample clean-up, followed by analysis on a triple quadrupole mass spectrometer in dynamic multiple reaction monitoring (dMRM) mode. 1,158 MRM transitions were used to quantify 525 glycopeptides and 151 peptides. An aliquot of the proteolytically digested was set aside to use as a control that was directly subjected to analysis on the triple quadrupole mass spectrometer.

For the AssayMap C18 workflow, which was designed to be fully automated, 30 μL of the proteolytically digested sample was transferred from a sample plate in a first position to another sample plate (e.g., Greiner clear v-bottomed plate) in a second position. The transferred sample was not mixed, and the sample plate was removed from the first position, sealed with a plate sealer, and placed in a 4° C. fridge. To a reagent plate (e.g., Greiner white u-bottomed plate, 50 μL of sample buffer (5% trifluoracetic acid (TFA) in water) was added and this plate was positioned in the first position. From this plate, 22 μL of sample buffer was transferred from the reagent plate to the sample Greiner clear v-bottomed plate in the second position. To the reagent plate, 200 μL of Mili-Q water was added. From this, 168 μL of Mili-Q water was transferred to the sample plate in the second position and mixed for 20 cycles.

For sample clean-up, the sample plate was moved from a second position to a third position, and a waste collection plate was placed in a fourth position. In a new 1.2 mL Abgene plate placed in the second position, 800 μL of equilibration/cartridge wash buffer (0.5% TFA in water) was added to each well. Then, in a second Abgene plate placed in a fifth position, 400 μL of priming/syringe wash buffer (50% Acetonitrile (ACN), 0.1% TFA in water) was added to each well. Next, the AssayMap C18 cartridges were placed onto the seating section and part 1 of the AssayMap C18 clean-up protocol was initiated. Sample loading amounts of 10 μg to 250 μg were evaluated.

First, an initial syringe wash was completed with a priming/syringe wash buffer (50% ACN, 0.1% TFA) for 4 wash cycles where the buffer does not flow through the C18 cartridge, followed by a priming step of flowing 100 μL of the priming/syringe wash buffer at 10 μL/min through the C18 cartridge, and an equilibration step of flowing 50 μL of the equilibration/cartridge wash buffer (0.5% TFA) at 10 μL/min through the C18 cartridge. Sample was then loaded onto the resin at a volume ranging from 100 μL to 200 μL at 3 μL/min. The cartridge was then subjected to an internal wash (200 μL at 3 μL/min-10 μL/min). Excess priming/syringe wash buffer was removed from the Abgene plate in the fifth position and replaced with 400 μL of fresh priming/syringe wash buffer in each well. To a new Greiner clear v-bottomed plate in a sixth position, 130 μL of elution buffer (80% ACN, 0.1% TFA in water) was added to each well. Next, a new Greiner clear v-bottomed plate was placed in a seventh position. Part 2 of the AssayMap C18 clean-up was then initiated, starting with a stringent syringe wash with 100 μL of the priming/syringe wash buffer for 2 wash cycles. Sample was then eluted with 100 μL of the elution buffer at 3 L/min so that the processed sample can be collected.

After completion of the AssayMap C18 clean-up protocol, the processed samples were dried using a SpeedVac evaporator and stored in a dried, sealed state at −20° C. until ready for LC-MS analysis. For the LC-MS analysis, samples were reconstituted and an internal standard (30 μL) was added to each sample well, which was then sealed with a thermal plate sealer. Samples were vortexed for 5 minutes on a plate shaker at 1400 RPM, sonicated for 1 minute in a water bath and centrifuged for 2 minutes at 4,700 RPM before being transferred to an Eppendorf plate for subsequent LC-MS analysis.

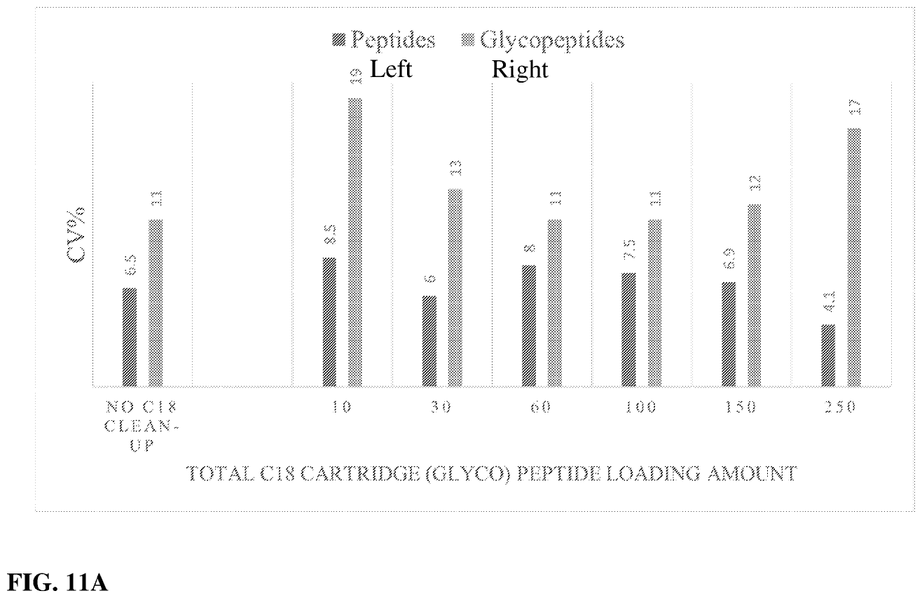

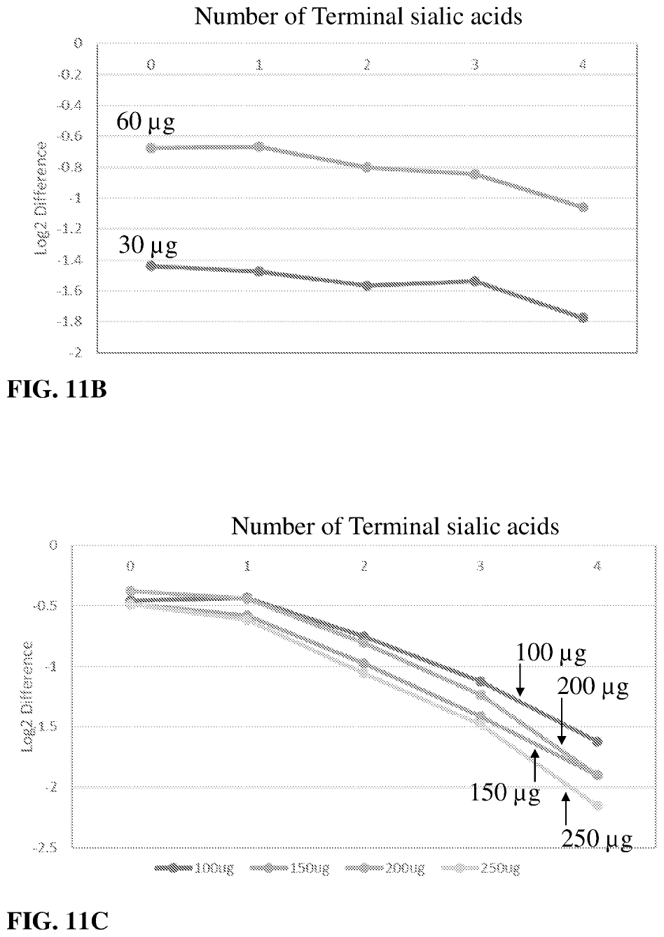

FIG. 11A shows the median CV % results for a sample not subjected to the sample clean-up procedure described above, and for sample loading amounts of 10 μg, 30 μg, 60 μg, 100 μg, 150 μg, and 250 μg. The coefficient of variation (CV) is a statistical measurement that describes the dispersion of the standard deviation in the data set, and is a key metric for assessing the quality and reproducibility of a given proteomic data set. A plurality of non-glycosylated peptides and glycopeptides were monitored via the MRM technique for sample cleaned with the C18 cartridge having different sample loading amounts. The CV % of four replicates was calculated from the area under the curve of the raw abundance peaks for the plurality of non-glycosylated peptides and glycopeptides. The CV % was less than 15% for glycopeptide sample loading between 30 μg and 150 μg which is a relatively low value that is difficult to achieve for glycopeptide analysis. For reference, recommended precision values of 15% for validating chromatographic assays can be found in “Bioanalytical Method Validation Guidance for Industry, U.S. Department of Health and Human Services, Food and Drug Administration, Center for Drug Evaluation and Research (CDER), Center for Veterinary Medicine (CVM), May 2018, Biopharmaceutics, p 24”. 60 μg and 100 μg sample loading amounts gave improved and comparable median CV % for both peptides and glycopeptides as compared to no sample clean-up and other sample loading amounts. Although the no C18 clean-up (control) showed a relatively low CV % compared to the C18 clean-up experiments at various loading amounts, it is worthwhile to note that the no C18 clean-up sample may contribute to excess salt being inputted into a MS that can cause a contamination problem resulting in operational downtime where an undesirable system maintenance procedure needs to be performed. Thus, under certain circumstances where the measurement process suffers an interference or contaminated mass spectrometer issue due to relatively low salt concentrations, the reverse-phase clean-up method described herein can have an advantage over the no C18 clean up. In addition, where the desired target glycopeptide for MS measurement is efficiently bound be the reverse-phase material and then efficiently eluted from the reverse-phase material, the reverse-phase clean-up method described herein can have an advantage over the no C18 clean up. FIGS. 11B and 11C show plots of the log₂ difference for sialylated glycopeptide species having various terminal sialic acids observed for various loading amounts. The log₂ difference corresponds to the log₂ (raw abundance values with C18 treatment—raw abundance values without C18) for all measured glycopeptides. The log₂ difference values of all the measured glycopeptides were categorized based on the number of sialic acid moieties such as 0, 1, 2, 3, or 4. A greater loss of sialylated glycopeptides was observed with increasing sample loading amounts. With the 30 μg and 60 μg sample loading amount, no significant loss of glycopeptides with less than 3 terminal sialic acids was observed when compared to non-sialylated glycopeptides.

### Example 8

This example demonstrates a method for processing a proteolytically digested sample to produce a processed sample suitable for use in a liquid chromatography-mass spectrometry (LC-MS) analysis, the method comprising subjecting a proteolytically digested sample to a solid phase extraction column comprising a C18 reversed-phase medium using a polypeptide loading concentration or a wash flow rate that results in improved peptide and glycopeptide analysis.

Proteolytic digestion of human serum samples were performed as described in Example 7. The AssayMap C18 workflow was as described in Example 7 and using the seven workflows described in Table 5. The LC-MS analysis was performed as described in Example 7. The sample loading amount of 60 μg was used for all workflows. A control (C) was performed by subjecting the proteolytically digested sample without sample clean-up to the LC-MS analysis.

TABLE 5
AssayMap workflows.

| Workflow | 1 | 2 | 3 | 4 | 5 | 6 | 7 |
|---|---|---|---|---|---|---|---|
| Sample buffer | 0.1% TFA | 0.5% TFA | 0.5% TFA | 0.5% TFA | 0.5% TFA | 0.5% TFA | 0.5% TFA |
| | | | 5% ACN | | | | |
| Equilibration buffer | 0.1% TFA | 0.1% TFA | 0.1% TFA | 0.5% TFA | 0.1% TFA | 0.1% TFA | 0.1% TFA |
| Loading volume | 100 μL | 100 μL | 100 μL | 100 μL | 200 μL | 2 × 100 μL | 100 μL |
| Loading flow rate | 3 μL/ | 3 μL/ | 3 μL/ | 3 μL/ | 3 μL/ | 3 μL/ | 3 μL/ |
| | minute | minute | minute | minute | minute | minute | minute |
| Washing flow rate | 10 μL/ | 10 μL/ | 10 μL/ | 10 μL/ | 10 μL/ | 10 μL/ | 3 μL/ |
| | minute | minute | minute | minute | minute | minute | minute |

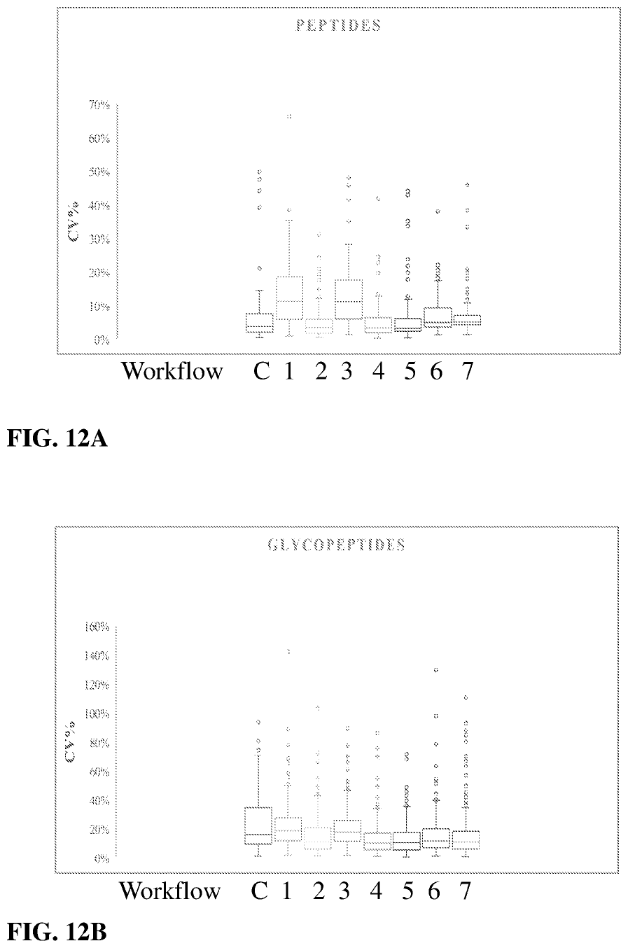

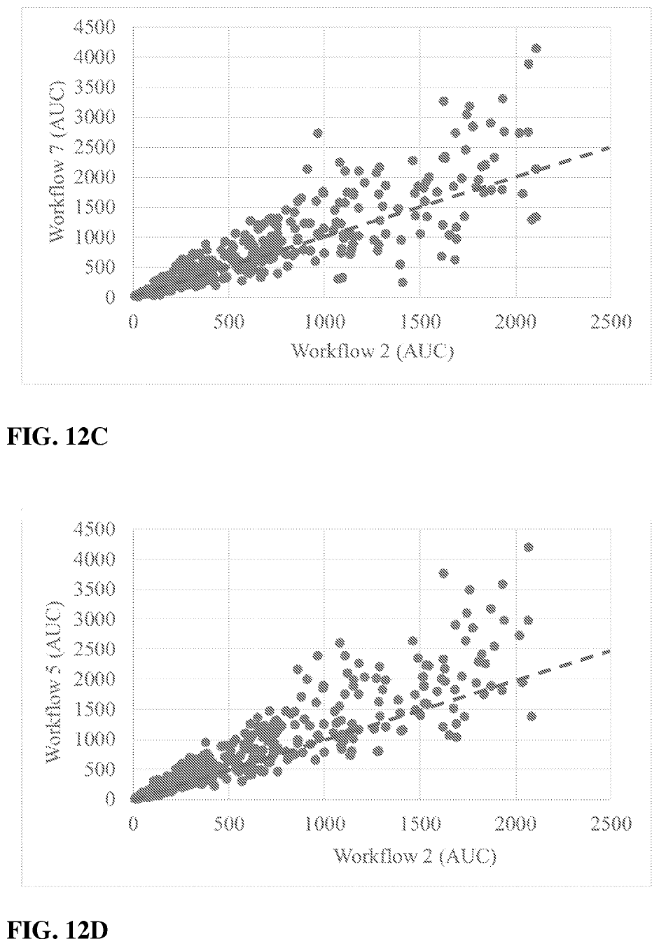

FIGS. 12A and 12B provide plots of CV % for the control (C) workflow and workflows 1-7 as observed for both peptides and glycopeptides monitored using the LC-MS analysis. For workflow 6, the 2×100 μL loading volume represents a 100 μL volume of sample flowed through the C18 cartridge, the outputted volume was collected, and then the same outputted volume was re-loaded by flowing it though the C18 cartridge. Workflows 2, 4, 5, and 7 have comparable CV % of glycopeptides and peptides to the workflow without C18 clean-up (control; C). FIGS. 12C and 12D provide unity plot comparisons between certain workflows (the unity line is indicated as a dashed line). In reference to FIG. 12C, the x-axis refers to area under the curve (AUC) values for raw abundance values measured for a panel of glycopeptides generated using Workflow 2 while the y-axis refers to AUC values measured for the same panel of glycopeptides generated using Workflow 7. Similarly, in FIG. 12D, the x-axis refers to AUC values measured for a panel of glycopeptides generated using Workflow 2 while the y-axis refers to AUC values measured for the same panel of glycopeptides generated using Workflow 5. As shown in FIG. 12C, workflow 7 provided a better recovery of low-to-mid-abundance glycopeptides as compared to workflow 2 thus demonstrating improved results with a washing flow rate of about 3 μL/minute. As shown in FIG. 12D, workflow 5 provided a better recovery of low-to-mid-abundance glycopeptides as compared to workflow 2 thus demonstrating improved results with a with a 200 μL loading volume that corresponds to a loading concentration of 0.3 μg/μL. For FIGS. 12C and 12D, the low-to-mid-abundance glycopeptides were the ones having an AUC of less than 2500 with respect to workflow 2.

### Example 9

This example demonstrates a method for processing a proteolytically digested sample to produce a processed sample suitable for use in a liquid chromatography-mass spectrometry (LC-MS) analysis, the method comprising subjecting a proteolytically digested sample to a solid phase extraction column comprising an RP-S reversed-phase medium using a polypeptide loading amount that results in improved peptide and glycopeptide analysis.

The AssayMAP 5 μL Reversed Phase (RP-S) cartridge (catalog no. G5496-60033; Agilent Technologies) was in a manner similar to Example 8. Proteolytic digestion of human serum samples were performed as described in Example 7. The LC-MS analysis was performed as described in Example 7. Sample loading amounts of 10 μg to 250 μg were evaluated.

As compared to a sample loading amount of 250 μg, 200 μg, 150 μg, and 10 μg, sample loading amounts of 100 μg and 60 μg demonstrated improved mean CV % of 13.21% and 13.66%, respectively, and improved median CV % of 9.53% and 10.25%, respectively. Table 6 provides the CV % obtained for specified sample loading amounts.

TABLE 6
CV % for sample loading amounts obtained
from RP-S sample clean-up.

| | 250 μg | 200 μg | 150 μg | 100 μg | 60 μg | 10 μg |
|---|---|---|---|---|---|---|
| Mean CV % | 16.5 | 13.49 | 16.18 | 13.21 | 13.66 | 24.39 |
| Median CV % | 12.67 | 10.08 | 13.80 | 9.53 | 10.25 | 21.73 |

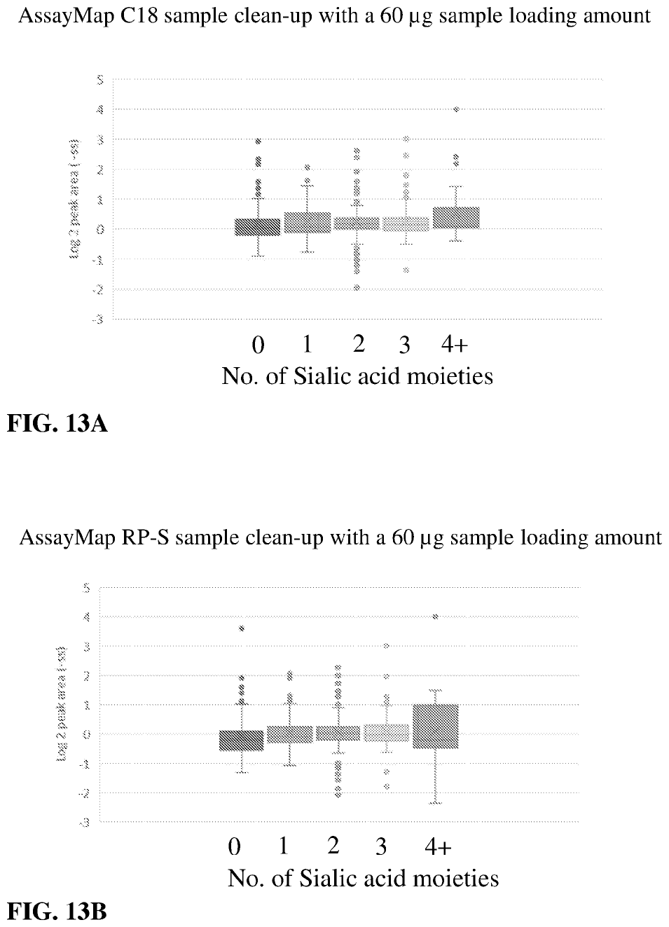

The AssayMap RP-S results obtained with a 60 μg loading amount were compared with results obtained with a 60 μg loading amount using an AssayMap C18 procedure described in Example 7. In FIG. 13A of the x-axis, the log₂ peak area corresponds to the log₂ (raw abundance peak area values with C18 treatment-raw abundance peak area values without C18) for all measured glycopeptides. In FIG. 13B of the x-axis, the log₂ peak area corresponds to the log₂ (raw abundance peak area values with RP-S treatment-raw abundance peak area values without RP-S treatment) for all measured glycopeptides. As shown in FIGS. 13A and 13B, the log₂ difference of peak area for analyzed sialyated glycopeptide species were comparable across varying numbers of sialic acid moieties in the glycan structure.

### Example 10

This example describes a method for quantifying biological markers from a serum sample comprising dried serum spot (DSS) sample collection followed by sample processing, and mass spectrometry (MS) analysis.

To compare the biological marker abundance of serum prepared from standard phlebotomy (venipuncture) and serum prepared from venipuncture blood, samples from a healthy individual were differentially prepared. Serum from finger-prick blood (˜50 μL) was spotted onto a Whatman 903 protein SaverCard (Cytiva) and allowed to dry for about >1 day at room temperature. After drying, 3 discs of 3 mm diameter were punched (i.e., removed) from each DSS card in preparation for sample extraction. DSS discs were placed in a new tube and resuspended in 100 μL of ammonium bicarbonate (50 mM) and dithiothreitol (DTT) (16.67 mM) solution. This solution was sonicated for 5 minutes before thermal denaturation, wherein samples were heated to 95° C. for 10 minutes, allowed to cool to ambient temperature for 5 minutes, and then heated to 60° C. for an additional 50 minutes. From this, 75 μL of denatured sample solution was transferred to a new tube. To this, 600 μL of ethanol (EtOH, 100%) was added and incubated overnight at −80° C. to precipitate protein content. Samples were then centrifuged at 20,000 relative centrifugal force (RCF) and dried. Ammonium bicarbonate and DTT solution was added to the dried material and incubated at 60° C. for 50 minutes to resolubilize protein content. The resulting solution was alkylated and reduced with an iodoacetamide (IAA, 90 mM) solution, followed by addition of a DTT solution. To the quenched (i.e., alkylated) biological samples and controls, 60 μL of the 0.167 μg/μL trypsin solution was added to each well. Digestion samples were incubated at 37° C. for 18 hours before formic acid was added to a 1% v/v for each sample to quench enzymatic digestion. Quenched samples were transferred to an injection plate and were subjected to LC-MS analysis, specifically MRM, to quantify peptide (i.e., marker) abundance for each processing method.

Serum derived from venipuncture or finger-prick (5 μL) was placed in a new tube. To this, 35 μL of ammonium bicarbonate (50 mM) was added. This solution was thermally denatured, wherein samples were heated to 100° C. for 5 minutes, allowed to cool to room temperature for 5 minutes, then mixed with 20 μL of 50 mM DTT, and then heated to 60° C. for an additional 50 minutes. The resulting solution was alkylated and reduced with 20 μL of 90 mM iodoacetamide (IAA) solution, followed by addition of 10 μL of a 50 mM DTT solution. To the quenched (i.e., alkylated) biological samples and controls, 60 μL of the 0.167 μg/μL trypsin solution was added to each well. Digestion samples were incubated at 37° C. for 18 hours before formic acid was added to a 1% v/v for each sample to quench enzymatic digestion. Quenched samples were transferred to an injection plate and were subjected to LC-MS analysis, specifically MRM, to quantify peptide (i.e., marker) abundance for each processing method.

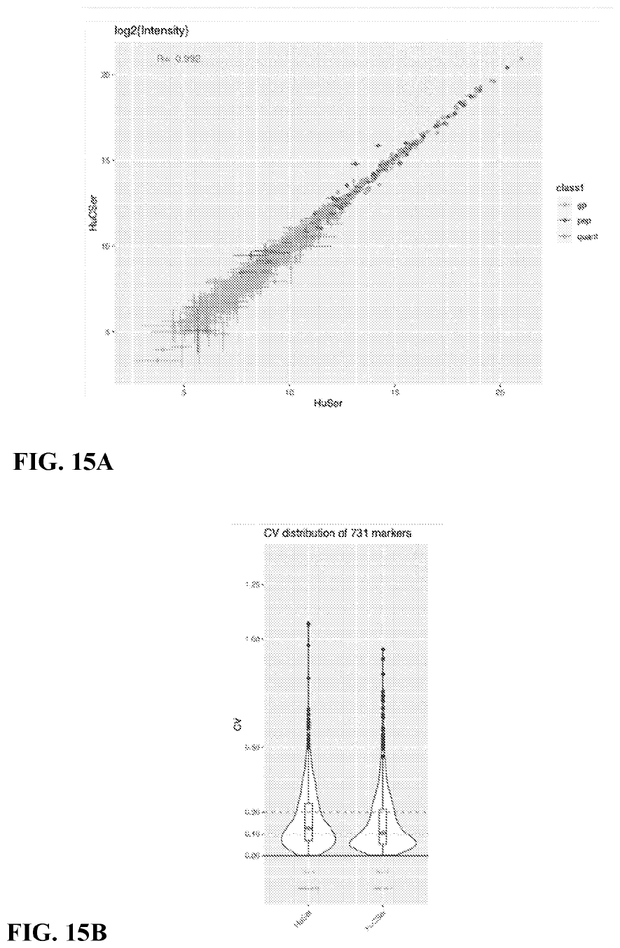

As shown in FIGS. 15A and 15B, comparison of serum prepared and processed from standard phlebotomy (venipuncture, HuSer) verses from finger-prick blood (HuCSer) showed significant correlation for peptide abundances (FIG. 15A), wherein R=0.992. In the legend, gp, pep, and quant represent glycopeptides, peptides, and other peptides, respectively. The two methods also showed comparable coefficients of variance (CV) (FIG. 15B) across the full proteomic dataset (731 markers). Thus, this work demonstrates finger-prick sample collection as a viable method for quantifying biological markers from a serum sample prepared from either a venipuncture or a finger-prick.

### Example 11

This example demonstrates a high correlation of biological marker abundance between two different serum absorbent or bibulous members, namely serum separated from finger-prick blood (HEMA; Hemaspot SE) and venous serum dried on a dried blood spot card (DSS) with capillary serum prepared from finger-prick blood and venous serum prepared from venipuncture collected blood, respectively.

All serum samples were collected from the same individual. To compare the biological marker abundance of serum prepared from serum separated from finger-prick blood (HEMA), serum prepared from capillary finger-prick blood (HuCSer), serum prepared from venous blood (HuSer), and venous serum dried on DBS card (DSS) were either spotted prior to processing or directly processed as described in Example 10.

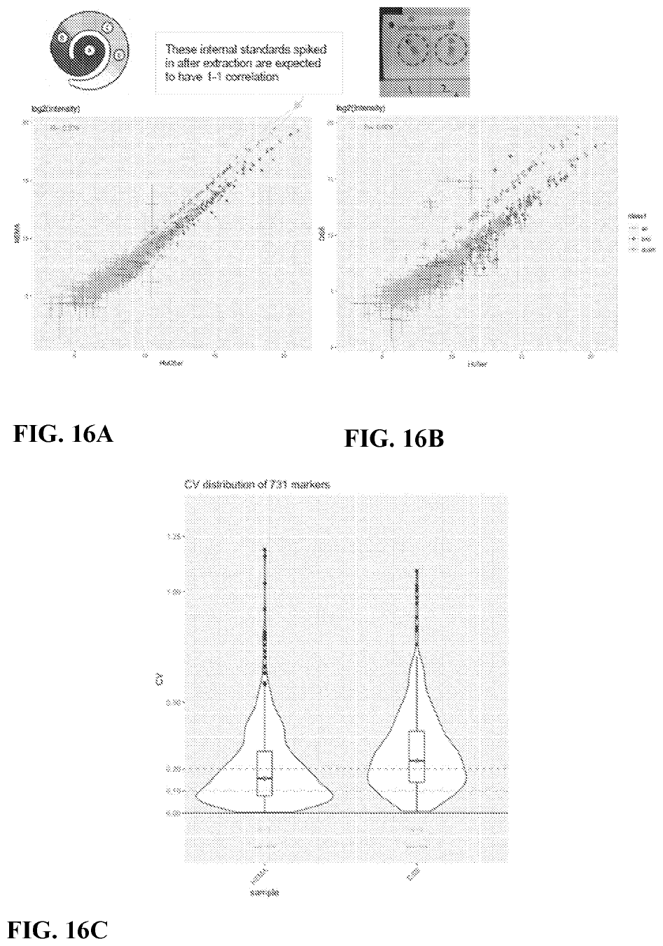

As shown in FIG. 16A, comparison of serum separated from finger-prick blood (HEMA) and serum prepared from capillary finger-prick blood (HuCSer) showed significant correlation for peptide abundances across the full proteomic dataset (Pearson's R˜0.98) and an acceptable median coefficient of variance (CV) of 0.16 between 3 replicates for 731 markers (FIG. 16C). As shown in FIG. 16B, comparison of venous serum (HuSer) and capillary serum dried on a DBS card (DSS) showed significant correlation for peptide abundances across the full proteomic dataset (Pearson's R˜0.93) and an acceptable median coefficient of variance (CV) of 0.24 between 3 replicates for 731 markers (FIG. 16C). In the legend of FIGS. 16A and 16B, gp, pep, and quant represent glycopeptides, peptides, and other peptides, respectively. Thus, this work demonstrates a high correlation of biological marker abundance between two different serum separating techniques, suggesting that DBS card separation and processing is a viable and cost-effective alternative.

### Example 12

This example demonstrates a dried blood spot (DBS) method for a LC-MS analysis of one or more glycopeptides from a blood spot card, wherein one or more extraction internal standards, including a polypeptide standard, is applied to the blood spot card prior to depositing a blood sample thereon. The polypeptide standard can include one or more polypeptides from SEQ ID NOS: 14-16, 18, and 22.

Blood spot cards are obtained having one or more extraction internal standards, including a polypeptide standard, deposited and dried thereon. The one or more extraction internal standards are applied in a known amount and in a known location on the dried blood spot card.

Whole blood samples are collected from individuals, e.g., by using a lancet to the finger or heel and applying the resulting blood to a delimited zone of a blood spot card. Replicate blood samples are obtained. After receipt of the blood spot cards, 3 or more discs of 3 mm diameter are punched and removed from each DBS card in preparation for sample extraction. DBS discs are placed in a new tube and resuspended in 100 μL of ammonium bicarbonate (50 mM) and dithiothreitol (DTT) (16.67 mM) solution. This solution is sonicated for 5 minutes before thermal denaturation, wherein samples are heated to 95° C. for 10 minutes, allowed to cool at ambient temperature for 5 minutes, and then are heated to 60° C. for an additional 50 minutes. From this, 75 μL of denatured sample solution is transferred to a new tube. To this, >600 μL of ethanol (EtOH, 100%) is added and incubated overnight to precipitate protein content. Samples are centrifuged at 20,000 RCF (10 minutes) and dried. Ammonium bicarbonate and DTT solution is added to the dried material and is incubated at 60° C. for 50 minutes to resolubilize protein content. The resulting solution is alkylated and reduced with an iodoacetamide (IAA, 90 mM) solution, followed by addition of a DTT solution. To the quenched (i.e., alkylated) biological samples, 60 μL of the 0.167 μg/μL trypsin solution is added to each well and another protein SEQ ID NO: 17. Because protein SEQ ID NO: 17. can be difficult to digest with trypsin, this protein is useful for monitoring the digestion efficiency by monitoring peptides generated from the digestion of protein SEQ ID NO: 17. Digestion samples are incubated at 37° C. for 18 hours before formic acid is added to 1% (v/v) for each sample to quench enzymatic digestion. To this, additional polypeptide standards, such as quantitative polypeptide standards, may be added. The samples are transferred to an injection plate, and are then subjected to LC-MS analysis.

### Example 13

This example demonstrates a DBS extraction and analysis method for measuring peptides and glycopeptides with LC-MS of an individual with advanced ovarian cancer and another individual with benign pelvic tumors. To compare results, serum samples were collected and analyzed in parallel using venous blood and DBS sample.

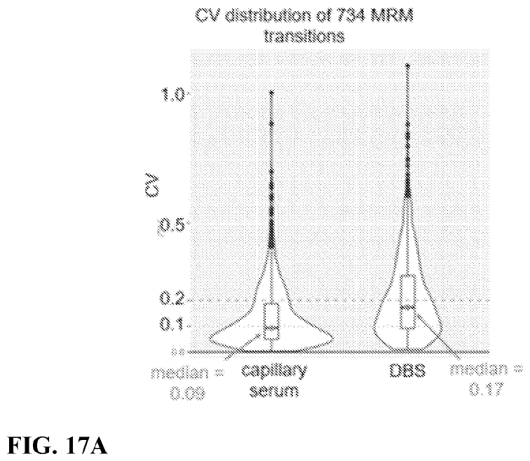

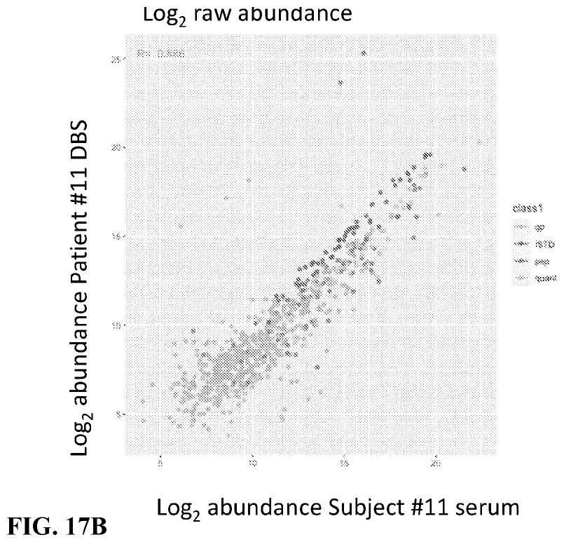

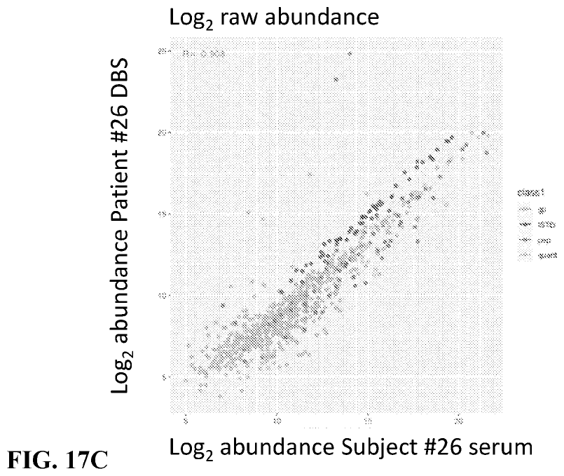

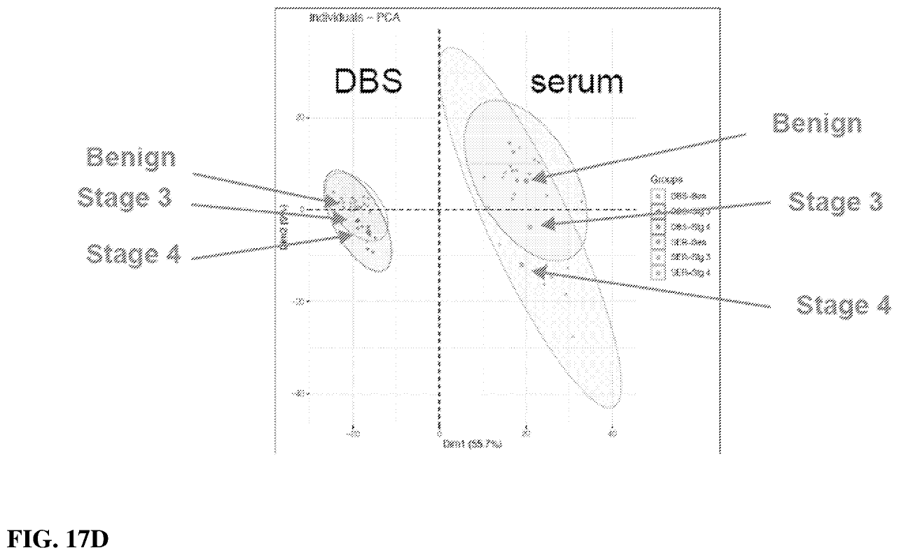

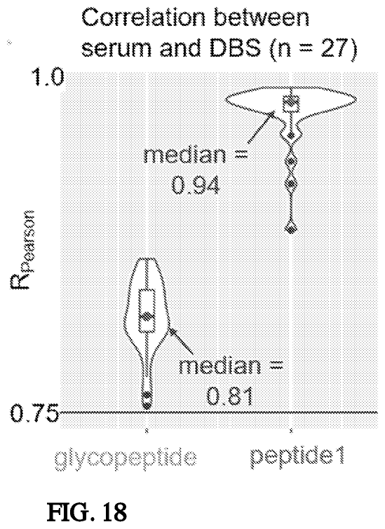

FIG. 17A shows a distribution of coefficient-of-variation (CV) values for each transition monitored for n=3 replicate digestions of capillary serum and DBS samples collected from the same individual. The median of the 734 CVs were 0.09 for capillary serum and 0.17 for DBS. Correlation amongst the sample set was performed for each marker, where 40 out of 530 analytes had R≥0.85. When considered individually for each patient sample, peptide abundances between DBS and serum samples (median R_{qpep}=0.94) are well correlated, suggesting glycoprotein extraction efficiency is not a major concern. As shown in FIG. 17B, healthy patient #11 had a correlation of Pearson R=0.89 for raw abundance, when compared to serum analysis. In FIG. 17C, advanced cancer patient #26 had a correlation of Pearson R=0.91 for raw abundance when compared to serum analysis. As shown in FIG. 18, a decreased correlation for glycopeptides (median R_{glyco}=0.81) compared to peptides may be a result of increased interference from sample matrix in DBS or lower signal-to-noise, due to lower transmission efficiency in triple quadrupole MS. Further, principal component analysis (PCA) of glycoproteomic signatures showed DBS and serum samples separated along first principal component, but also separated by malignancy along the same axis in the second component, demonstrating the ability to discriminate between benign and malignant tumor samples (FIG. 17D), using either approach.

Thus, this study demonstrates a DBS extraction and analysis method for discriminating between malignant and benign tumors based upon a glycoproteomic signature.

### Example 14

An MRM analysis was performed on DBS samples from advanced ovarian cancer (malignant pelvic tumor) and control (benign pelvic tumor) patients. Relative abundances of 17 glycopeptides were found to be significantly different between the malignant and benign pelvic tumor samples without adjusting for false discovery rate (FDR). The glycoproteins associated with these glycopeptides are summarized in Table 8. The amino acid sequences and other structural characteristics of the glycopeptides having significantly fold changes are provided in Table 9. In Table 10, the glycan structures for these glycopeptides are provided, with an associated glycan structure legend. LC-MRM-MS parameters for the peptide structures are summarized in Table 11.

TABLE 8
Glycoproteins associated with pelvic tumors

| SEQ | | | | |
|---|---|---|---|---|
| ID | Protein | Protein | Uniprot | |
| NO | Abbreviation | Name | ID | Protein Sequence |
| 23 | AACT | Alpha-1- | P01011 | MERMLPLLALGLLAAGFCPAVLCHPNSPLD |
| | | antichymotrypsin | | EENLTQENQDRGTHVDLGLASANVDFAFSL |
| | | | | YKQLVLKAPDKNVIFSPLSISTALAFLSLGAH |
| | | | | NTTLTEILKGLKFNLTETSEAEIHQSFQHLLR |
| | | | | TLNQSSDELQLSMGNAMFVKEQLSLLDRFT |
| | | | | EDAKRLYGSEAFATDFQDSAAAKKLINDYV |
| | | | | KNGTRGKITDLIKDLDSQTMMVLVNYIFFK |
| | | | | AKWEMPFDPQDTHQSRFYLSKKKWVMVP |
| | | | | MMSLHHLTIPYFRDEELSCTVVELKYTGNAS |
| | | | | ALFILPDQDKMEEVEAMLLPETLKRWRDSL |
| | | | | EFREIGELYLPKFSISRDYNLNDILLQLGIEEA |
| | | | | FTSKADLSGITGARNLAVSQVVHKAVLDVF |
| | | | | EEGTEASAATAVKITLLSALVETRTIVRFNRP |
| | | | | FLMIIVPTDTQNIFFMSKVTNPKQA |
| 24 | CLUS | Clusterin | P10909 | MMKTLLLFVGLLLTWESGQVLGDQTVSDN |
| | | | | ELQEMSNQGSKYVNKEIQNAVNGVKQIKTL |
| | | | | IEKTNEERKTLLSNLEEAKKKKEDALNETRES |
| | | | | ETKLKELPGVCNETMMALWEECKPCLKQT |
| | | | | CMKFYARVCRSGSGLVGRQLEEFLNQSSPFY |
| | | | | FWMNGDRIDSLLENDRQQTHMLDVMQDH |
| | | | | FSRASSIIDELFQDRFFTREPQDTYHYLPFSLP |
| | | | | HRRPHFFFPKSRIVRSLMPFSPYEPLNFHAM |
| | | | | FQPFLEMIHEAQQAMDIHFHSPAFQHPPTE |
| | | | | FIREGDDDRTVCREIRHNSTGCLRMKDQCD |
| | | | | KCREILSVDCSTNNPSQAKLRRELDESLQVA |
| | | | | ERLTRKYNELLKSYQWKMLNTSSLLEQLNE |
| | | | | QFNWVSRLANLTQGEDQYYLRVTTVASHT |
| | | | | SDSDVPSGVTEVVVKLFDSDPITVTVPVEVS |
| | | | | RKNPKFMETVAEKALQEYRKKHREE |
| 25 | KLKB1 | Plasma | P03952 | MILFKQATYFISLFATVSCGCLTQLYENAFFR |
| | | kallikrein | | GGDVASMYTPNAQYCQMRCTFHPRCLLFS |
| | | | | FLPASSINDMEKRFGCFLKDSVTGTLPKVHR |
| | | | | TGAVSGHSLKQCGHQISACHRDIYKGVDM |
| | | | | RGVNFNVSKVSSVEECQKRCTNNIRCQFFSY |
| | | | | ATQTFHKAEYRNNCLLKYSPGGTPTAIKVLS |
| | | | | NVESGFSLKPCALSEIGCHIMNIFQHLAFSDV |
| | | | | DVARVLTPDAFVCRTICTYHPNCLFFTFYTN |
| | | | | VWKIESQRNVCLLKTSESGTPSSSTPQENTIS |
| | | | | GYSLLTCKRTLPEPCHSKIYPGVDFGGEELN |
| | | | | VTFVKGVNVCQETCTKMIRCQFFTYSLLPE |
| | | | | DCKEEKCKCFLRLSMDGSPTRIAYGTQGSSG |
| | | | | YSLRLCNTGDNSVCTTKTSTRIVGGTNSSWG |
| | | | | EWPWQVSLQVKLTAQRHLCGGSLIGHQW |
| | | | | VLTAAHCFDGLPLQDVWRIYSGILNLSDITK |
| | | | | DTPFSQIKEIIIHQNYKVSEGNHDIALIKLQA |
| | | | | PLNYTEFQKPICLPSKGDTSTIYTNCWVTG |
| | | | | WGFSKEKGEIQNILQKVNIPLVTNEECQKR |
| | | | | YQDYKITQRMVCAGYKEGGKDACKGDSGG |
| | | | | PLVCKINGMWRLVGITSWGEGCARREQP |
| | | | | GVYTKVAEYMDWILEKTQSSDGKAQMQSP |
| | | | | A |
| 26 | KNG1 | Kininogen-1 | P01042 | MKLITILFLCSRLLLSLTQESQSEEIDCNDKDL |
| | | | | FKAVDAALKKYNSQNQSNNQFVLYRITEAT |
| | | | | KTVGSDTFYSFKYEIKEGDCPVQSGKTWQD |
| | | | | CEYKDAAKAATGECTATVGKRSSTKFSVAT |
| | | | | QTCQITPAEGPVVTAQYDCLGCVHPISTQS |
| | | | | PDLEPILRHGIQYFNNNTQHSSLFMLNEVKR |
| | | | | AQRQVVAGLNFRITYSIVQTNCSKENFLFLT |
| | | | | PDCKSLWNGDTGECTDNAYIDIQLRIASFSQ |
| | | | | NCDIYPGKDFVQPPTKICVGCPRDIPTNSPE |
| | | | | LEETLTHTITKLNAENNATFYFKIDNVKKAR |
| | | | | VQVVAGKKYFIDFVARETTCSKESNEELTES |
| | | | | CETKKLGQSLDCNAEVYVVPWEKKIYPTVN |
| | | | | CQPLGMISLMKRPPGFSPFRSSRIGEIKEETT |
| | | | | VSPPHTSMAPAQDEERDSGKEQGHTRRHD |
| | | | | WGHEKQRKHNLGHGHKHERDQGHGHQR |
| | | | | GHGLGHGHEQQHGLGHGHKFKLDDDLEH |
| | | | | QGGHVLDHGHKHKHGHGHGKHKNKGKK |
| | | | | NGKHNGWKTEHLASSSEDSTTPSAQTQEKT |
| | | | | EGPTPIPSLAKPGVTVTFSDFQDSDLIATMM |
| | | | | PPISPAPIQSDDDWIPDIQIDPNGLSFNPISDF |
| | | | | PDTTSPKCPGRPWKSVSEINPTTQMKESYYF |
| | | | | DLTDGLS |
| 27 | VTNC | Vitronectin | P04004 | MAPLRPLLILALLAWVALADQESCKGRCTE |
| | | | | GFNVDKKCQCDELCSYYQSCCTDYTAECKP |
| | | | | QVTRGDVFTMPEDEYTVYDDGEEKNNATV |
| | | | | HEQVGGPSLTSDLQAQSKGNPEQTPVLKPE |
| | | | | EEAPAPEVGASKPEGIDSRPETLHPGRPQPP |
| | | | | AEEELCSGKPFDAFTDLKNGSLFAFRGQYCY |
| | | | | ELDEKAVRPGYPKLIRDVWGIEGPIDAAFT |
| | | | | RINCQGKTYLFKGSQYWRFEDGVLDPDYPR |
| | | | | NISDGFDGIPDNVDAALALPAHSYSGRERV |
| | | | | YFFKGKQYWEYQFQHQPSQEECEGSSLSAV |
| | | | | FEHFAMMQRDSWEDIFELLFWGRTSAGTR |
| | | | | QPQFISRDWHIGVPGQVDAAMAGRIYISGM |
| | | | | APRPSLAKKQRFRHRNRKGYRSQRGHSRGR |
| | | | | NQNSRRPSRATWLSLESSEESNLGANNYDD |
| | | | | YRMDWLVPATCEPIQSVFFFSGDKYYRVNL |
| | | | | RTRRVDTVDPPYPRSIAQYWLGCPAPGHL |
| 28 | A2MG | Alpha-2- | P01023 | MGKNKLLHPSLVLLLLVLLPTDASVSGKPQY |
| | | macroglobulin | | MVLVPSLLHTETTEKGCVLLSYLNETVTVSA |
| | | | | SLESVRGNRSLFTDLEAENDVLHCVAFAVPK |
| | | | | SSSNEEVMFLTVQVKGPTQEFKKRTTVMVK |
| | | | | NEDSLVFVQTDKSIYKPGQTVKFRVVSMDE |
| | | | | NFHPLNELIPLVYIQDPKGNRIAQWQSFQLE |
| | | | | GGLKQFSFPLSSEPFQGSYKVVVQKKSGGRT |
| | | | | EHPFTVEEFVLPKFEVQVTVPKIITILEEEMN |
| | | | | VSVCGLYTYGKPVPGHVTVSICRKYSDASD |
| | | | | CHGEDSQAFCEKFSGQLNSHGCFYQQVKTK |
| | | | | VFQLKRKEYEMKLHTEAQIQEEGTVVELTG |
| | | | | RQSSEITRTITKLSFVKVDSHFRQGIPFFGQV |
| | | | | RLVDGKGVPIPNKVIFIRGNEANYYSNATTD |
| | | | | EHGLVQFSINTTNVMGTSLTVRVNYKDRSP |
| | | | | CYGYQWVSEEHEEAHHTAYLVFSPSKSFVH |
| | | | | LEPMSHELPCGHTQTVQAHYILNGGTLLGL |
| | | | | KKLSFYYLIMAKGGIVRTGTHIGLLVKQEDM |
| | | | | KGHFSISIPVKSDIAPVARLLIYAVLPTGDVI |
| | | | | GDSAKYDVENCLANKVDLSFSPSQSLPASHA |
| | | | | HLRVTAAPQSVCALRAVDQSVLLMKPDAE |
| | | | | LSASSVYNLLPEKDLTGFPGPLNDQDNEDCI |
| | | | | NRHNVYINGITYTPVSSTNEKDMYSFLEDM |
| | | | | GLKAFTNSKIRKPKMCPQLQQYEMHGPEGL |
| | | | | RVGFYESDVMGRGHARLVHVEEPHTETVR |
| | | | | KYFPETWIWDLVVVNSAGVAEVGVTVPDT |
| | | | | ITEWKAGAFCLSEDAGLGISSTASLRAFQPFF |
| | | | | VELTMPYSVIRGEAFTLKATVLNYLPKCIRV |
| | | | | SVQLEASPAFLAVPVEKEQAPHICICANGRQ |
| | | | | TVSWAVTPKSLGNVNFTVSAEALESQELCG |
| | | | | TEVPSVPEHGRKDTVIKPLLVEPEGLEKETT |
| | | | | FNSLLCPSGGEVSEELSLKLPPNVVEESARAS |
| | | | | VSVLGDILGSAMQNTQNLLQMPYGCGEQN |
| | | | | MVLFAPNIYVLDYLNETQQLTPEIKSKAIGY |
| | | | | LNTGYQRQLNYKHIYDGSYSTFGERYGRNQ |
| | | | | GNTWLTAFVLKTFAQARAYIFIDEAHITQA |
| | | | | LIWLSQRQKDNGCFRSSGSLLNNAIKGGVE |
| | | | | DEVTLSAYITIALLEIPLTVTHPVVRNALFCL |
| | | | | ESAWKTAQEGDHIGSHVYTKALLAYAFALA |
| | | | | GNQDKRKEVLKSLNEEAVKKDNSVHWERP |
| | | | | QKPKAPVGHFYEPQAPSAEVEMTSYVLLAY |
| | | | | LTAQPAPTSEDLTSATNIVKWITKQQNAQG |
| | | | | GFSSTQDTVVALHALSKYGAATFTRTGKAA |
| | | | | QVTIQSSGTFSSKFQVDNNNRLLLQQVSLPE |
| | | | | LPGEYSMKVTGEGGVYLQTSLKYNILPEKEE |
| | | | | FPFALGVQTLPQTCDEPKAHTSFQISLSVSYT |
| | | | | GSRSASNMAIVDVKMVSGFIPLKPTVKMLE |
| | | | | RSNHVSRTEVSSNHVLIYLDKVSNQTLSLFFT |
| | | | | VLQDVPVRDLKPAIVKVYDYYETDEFAIAE |
| | | | | YNAPCSKDLGNA |
| 29 | AGP12 | Alpha-1- | P02763/ | 1: |
| | | acid | P19652 | MALSWVLTVLSLLPLLEAQIPLCANLVPVPI |
| | | glycoprotein | | TNATLDQITGKWFYIASAFRNEEYNKSVQEI |
| | | 1 / 2 | | QATFFYFTPNKTEDTIFLREYQTRQDQCIYN |
| | | | | TTYLNVQRENGTISRYVGGQEHFAHLLILR |
| | | | | DTKTYMLAFDVNDEKNWGLSVYADKPETT |
| | | | | KEQLGEFYEALDCLRIPKSDVVYTDWKKDK |
| | | | | CEPLEKQHEKERKQEEGES |
| | | | | 2: |
| | | | | MALSWVLTVLSLLPLLEAQIPLCANLVPVPI |
| | | | | TNATLDRITGKWFYIASAFRNEEYNKSVQEI |
| | | | | QATFFYFTPNKTEDTIFLREYQTRQNQCFYN |
| | | | | SSYLNVQRENGTVSRYEGGREHVAHLLFLR |
| | | | | DTKTLMFGSYLDDEKNWGLSFYADKPETTK |
| | | | | EQLGEFYEALDGLCIPRSDVMYTDWKKDKC |
| | | | | EPLEKQHEKERKQEEGES |
| 30 | APOC3 | Apolipoprotein | P02656 | MQPRVLLVVALLALLASARASEAEDASLLSF |
| | | C- | | MQGYMKHATKTAKDALSSVQESQVAQQA |
| | | III | | RGWVTDGFSSLKDYWSTVKDKFSEFWDLD |
| | | | | PEVRPTSAVAA |
| 31 | CERU | Ceruloplasmin | P00450 | MKILILGIFLFLCSTPAWAKEKHYYIGIIETT |
| | | | | WDYASDHGEKKLISVDTEHSNIYLQNGPDR |
| | | | | IGRLYKKALYLQYTDETFRTTIEKPVWLGFL |
| | | | | GPIIKAETGDKVYVHLKNLASRPYTFHSHGI |
| | | | | TYYKEHEGATYPDNTTDFQRADDKVYPGE |
| | | | | QYTYMLLATEEQSPGEGDGNCVTRIYHSHI |
| | | | | DAPKDIASGLIGPLIICKKDSLDKEKEKHIDR |
| | | | | EFVVMFSVVDENFSWYLEDNIKTYCSEPEK |
| | | | | VDKDNEDFQESNRMYSVNGYTFGSLPGLSM |
| | | | | CAEDRVKWYLFGMGNEVDVHAAFFHGQA |
| | | | | LTNKNYRIDTINLFPATLFDAYMVAQNPGE |
| | | | | WMLSCQNLNHLKAGLQAFFQVQECNKSSS |
| | | | | KDNIRGKHVRHYYIAAEEIIWNYAPSGIDIF |
| | | | | TKENLTAPGSDSAVFFEQGTTRIGGSYKKLV |
| | | | | YREYTDASFTNRKERGPEEEHLGILGPVIWA |
| | | | | EVGDTIRVTFHNKGAYPLSIEPIGVRFNKNN |
| | | | | EGTYYSPNYNPQSRSVPPSASHVAPTETFTY |
| | | | | EWTVPKEVGPTNADPVCLAKMYYSAVDPT |
| | | | | KDIFTGLIGPMKICKKGSLHANGRQKDVDK |
| | | | | EFYLFPTVFDENESLLLEDNIRMFTTAPDQV |
| | | | | DKEDEDFQESNKMHSMNGFMYGNQPGLT |
| | | | | MCKGDSVVWYLFSAGNEADVHGIYFSGNT |
| | | | | YLWRGERRDTANLFPQTSLTLHMWPDTEG |
| | | | | TFNVECLTTDHYTGGMKQKYTVNQCRRQS |
| | | | | EDSTFYLGERTYYLAAVEVEWDYSPQREWE |
| | | | | KELHHLQEQNVSNAFLDKGEFYIGSKYKKV |
| | | | | VYRQYTDSTFRVPVERKAEEEHLGILGPQL |
| | | | | HADVGDKVKIIFKNMATRPYSIHAHGVQTE |
| | | | | SSTVTPTLPGETLTYVWKIPERSGAGTEDSA |
| | | | | CIPWAYYSTVDQVKDLYSGLIGPLIVCRRPY |
| | | | | LKVFNPRRKLEFALLFLVFDENESWYLDDNI |
| | | | | KTYSDHPEKVNKDDEEFIESNKMHAINGRM |
| | | | | FGNLQGLTMHVGDEVNWYLMGMGNEIDL |
| | | | | HTVHFHGHSFQYKHRGVYSSDVFDIFPGTY |
| | | | | QTLEMFPRTPGIWLLHCHVTDHIHAGMET |
| | | | | TYTVLQNEDTKSG |
| 32 | HPT | Haptoglobin | P00738 | MSALGAVIALLLWGQLFAVDSGNDVTDIA |
| | | | | DDGCPKPPEIAHGYVEHSVRYQCKNYYKLR |
| | | | | TEGDGVYTLNDKKQWINKAVGDKLPECEA |
| | | | | DDGCPKPPEIAHGYVEHSVRYQCKNYYKLR |
| | | | | TEGDGVYTLNNEKQWINKAVGDKLPECEA |
| | | | | VCGKPKNPANPVQRILGGHILDAKGSFPWQ |
| | | | | AKMVSHHNLTTGATLINEQWLLTTAKNLFL |
| | | | | NHSENATAKDIAPTLTLYVGKKQLVEIEKV |
| | | | | VLHPNYSQVDIGLIKLKQKVSVNERVMPICL |
| | | | | PSKDYAEVGRVGYVSGWGRNANFKFTDHL |
| | | | | KYVMLPVADQDQCIRHYEGSTVPEKKTPKS |
| | | | | PVGVQPILNEHITFCAGMSKYQEDTCYGDA |
| | | | | GSAFAVHDLEEDTWYATGILSFDKSCAVAE |
| | | | | YGVYVKVTSIQDWVQKTIAEN |
| 33 | IC1 | Plasma | P05155 | MASRLTLLTLLLLLLAGDRASSNPNATSSSSQ |
| | | protease | | DPESLQDRGEGKVATTVISKMLFVEPILEVSS |
| | | C1 | | LPTTNSTTNSATKITANTTDEPTTQPTTEPTT |
| | | inhibitor | | QPTIQPTQPTTQLPTDSPTQPTTGSFCPGPV |
| | | | | TLCSDLESISTEAVLGDALVDFSLKLYHAFS |
| | | | | AMKKVETNMAFSPFSIASLLTQVLLGAGEN |
| | | | | TKTNLESILSYPKDFTCVHQALKGFTTKGVT |
| | | | | SVSQIFHSPDLAIRDTFVNASRTLYSSSPRVLS |
| | | | | NNSDANLELINTWVAKNTNNKISRLLDSLPS |
| | | | | DTRLVLLNAIYLSAKWKTTFDPKKTRMEPF |
| | | | | HFKNSVIKVPMMNSKKYPVAHFIDQTLKAK |
| | | | | VGQLQLSHNLSLVILVPQNLKHRLEDMEQA |
| | | | | LSPSVFKAIMEKLEMSKFQPTLLTLPRIKVTT |
| | | | | SQDMLSIMEKLEFFDFSYDLNLCGLTEDPDL |
| | | | | QVSAMQHQTVLELTETGVEAAAASAISVAR |
| | | | | TLLVFEVQQPFLFVLWDQQHKFPVFMGRV |
| | | | | YDPRA |
| 34 | IGG2 | Immunoglobulin | P01859 | ASTKGPSVFPLAPCSRSTSESTAALGCLVKDY |
| | | heavy | | FPEPVTVSWNSGALTSGVHTFPAVLQSSGLY |
| | | constant | | SLSSVVTVPSSNFGTQTYTCNVDHKPSNTKV |
| | | gamma 2 | | DKTVERKCCVECPPCPAPPVAGPSVFLFPPK |
| | | | | PKDTLMISRTPEVTGVVVDVSHEDPEVQFN |
| | | | | WYVDGVEVHNAKTKPREEQFNSTFRVVSV |
| | | | | LTVVHQDWLNGKEYKCKVSNKGLPAPIEK |
| | | | | TISKTKGQPREPQVYTLPPSREEMTKNQVSL |
| | | | | TCLVKGFYPSDISVEWESNGQPENNYKTTP |
| | | | | PMLDSDGSFFLYSKLTVDKSRWQQGNVFSC |
| | | | | SVMHEALHNHYTQKSLSLSPGK |

TABLE 3
Peptide structures associated with pelvic tumors

| | | | Linking | Linking | |
|---|---|---|---|---|---|
| | | | Site Pos. | Site Pos. | |
| SEQ | Peptide Structure (PS) | | in Protein | in Peptide | Glycan |
| ID NO | NAME | Peptide Sequence | Sequence | Sequence | Structure |
| 35 | AACT_127_5401 | TLNQSSDELQL | 127 | 3 | 5401 |
| | | SMGNAMFVK | | | |
| 36 | AACT_127_5402 | TLNQSSDELQL | 127 | 3 | 5402 |
| | | SMGNAMFVK | | | |
| 37 | AACT_271_6503 | YTGNASALFIL | 271 | 4 | 6503 |
| | | PDQDK | | | |
| 38 | CLUS_291_5401 | HNSTGCLR | 291 | 2 | 5401 |
| 39 | KLKB1_308_5402 | IYPGVDFGGEE | 308 | 13 | 5402 |
| | | LNVTFVK | | | |
| 40 | KNG1_205_6513 | ITYSIVQTNCSK | 205 | 9 | 6513 |
| 41 | VTNC_242_6503 | NISDGFDGIPD | 242 | 1 | 6503 |
| | | NVDAALALPA | | | |
| | | HSYSGR | | | |
| 42 | VTNC_242_6513 | NISDGFDGIPD | 242 | 1 | 6513 |
| | | NVDAALALPA | | | |
| | | HSYSGR | | | |
| 43 | CERU_762_6513 | ELHHLQEQNV | 762 | 9 | 6513 |
| | | SNAFLDK | | | |
| 44 | AGP12_72_6513 | SVQEIQATFFY | 72 | 15 | 6513 |
| | | FTPNK | | | |
| 45 | IGG2_297_5410 | EEQFNSTFR | 297 | 5 | 5410 |
| 46 | A2MG_869_5200 | SLGNVNFTVSA | 869 | 6 | 5200 |
| | | EALESQELCGT | | | |
| | | EVPSVPEHGR | | | |
| 47 | AACT_106_5402 | FNLTETSEAEIH | 106 | 2 | 5402 |
| | | QSFQHLLR | | | |
| 48 | APOC3_74_1101 | FSEFWDLDPEV | 94 | 14 | 1101 |
| | | RPTSAVAA | | | |
| 49 | IC1_352_5402 | VGQLQLSHNLS | 352 | 9 | 5402 |
| | | LVILVPQNLK | | | |
| 50 | HPT_241_6513 | VVLHPNYSQV | 241 | 6 | 6513 |
| | | DIGLIK | | | |
| 51 | AGP12_72_7613 | SVQEIQATFFY | 72 | 15 | 7613 |
| | | FTPNK | | | |

TABLE 10
Glycan structure (GL NO), structure, and composition

| Glycan Structure GL NO. | Structure | Composition |
|---|---|---|
| 1101 | 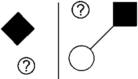 | Hex(1)HexNAc(1)Fuc(0)NeuAc(1) |
| 5200 | 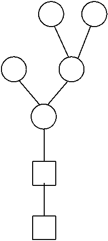 | Hex(5)HexNAc(2)Fuc(0)NeuAc(0) |
| 5401 | 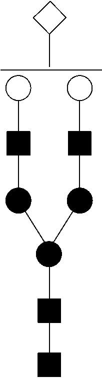 | Hex(5)HexNAc(4)Fuc(0)NeuAc(1) |
| 5402 | 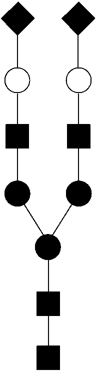 | Hex(5)HexNAc(4)Fuc(0)NeuAc(2) |
| 5410 | 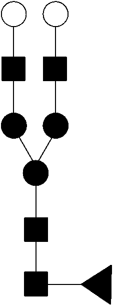 | Hex(5)HexNAc(4)Fuc(1)NeuAc(0) |
| 6503 | 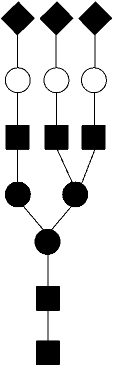 | Hex(6)HexNAc(5)Fuc(0)NeuAc(3) |
| 6513 | 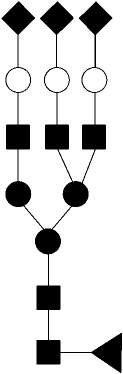 | Hex(6)HexNAc(5)Fuc(1)NeuAc(3) |
| 7613 | 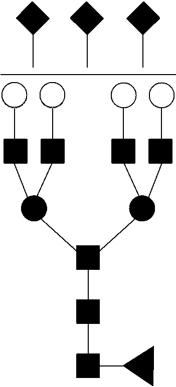 | Hex(7)HexNAc(6)Fuc(1)NeuAc(3) |

Legend for Table 10

[symbol]

Glc

[symbol]

Gal

[symbol]

Man

[symbol]

Fuc

[symbol]

NeuSAc

[symbol]

GlcNAc

[symbol]

GalNAc

[symbol]

ManNAc

TABLE 11
LC-MS parameters for MRM monitoring of peptide
structures associated with pelvic tumors

| | | Collision | | | 1^{st} | 1^{st} |
|---|---|---|---|---|---|---|
| SEQ | RT | Energy | Precursor | Precursor | product | product |
| ID NO | (min) | (V) | m/z | charge | m/z | charge |
| 35 | 33.080 | 20 | 1032.9 | 4 | 366.1 | 1 |
| 36 | 33.904 | 30 | 1105.7 | 4 | 204.1 | 1 |
| 37 | 31.496 | 25 | 1154.7 | 4 | 274.1 | 1 |
| 38 | 4.948 | 24 | 953.4 | 3 | 366.1 | 1 |
| 39 | 37.878 | 18 | 1048.4 | 4 | 1094.0 | 2 |
| 40 | 16.902 | 25 | 1106.4 | 4 | 274.1 | 1 |
| 41 | 37.750 | 25 | 1127.9 | 5 | 366.1 | 1 |
| 42 | 37.748 | 30 | 1157.1 | 5 | 274.1 | 1 |
| 43 | 20.800 | 30 | 1258.5 | 4 | 274.1 | 1 |
| 44 | 37.504 | 25 | 1324.3 | 4 | 366.1 | 1 |
| 45 | 12.728 | 20 | 976.1 | 3 | 366.1 | 1 |
| 46 | 34.426 | 23 | 1158.8 | 4 | 1206.9 | 3 |
| 47 | 37.156 | 30 | 922.2 | 5 | 204.1 | 1 |
| 48 | 37.654 | 22 | 931.8 | 3 | 274.1 | 1 |
| 49 | 39.378 | 35 | 1130.8 | 4 | 204.1 | 1 |
| 50 | 31.006 | 30 | 1201.5 | 4 | 366.1 | 1 |
| 51 | 37.504 | 25 | 1324.3 | 4 | 366.1 | 1 |

To demonstrate the statistical significance in the peptide structure relative abundance difference between the malignant and benign pelvic tumor populations, the fold changes, p-values, and false discovery rates (FDR) are provided in Table 12. Fold-changes for individual glycopeptides were calculated on normalized relative abundances of malignant vs. benign pelvic tumor samples. False discovery rate was calculated using the Benjamini-Hochberg method.

TABLE 12
Differential marker expression analysis
for malignant vs. benign pelvic tumors

| SEQ | malignant/benign | malignant/benign | malignant/benign |
|---|---|---|---|
| ID NO | fold change | p-value | FDR |
| 35 | 0.468 | 2.04e−05 | 1.08e−02 |
| 36 | 0.676 | 4.57e−04 | 6.50e−02 |
| 37 | 0.523 | 1.11e−03 | 6.50e−02 |
| 38 | 1.392 | 2.32e−03 | 7.24e−02 |
| 39 | 1.646 | 7.94e−03 | 1.00e−01 |
| 40 | 1.413 | 9.09e−03 | 1.03e−01 |
| 41 | 0.664 | 8.97e−04 | 6.50e−02 |
| 42 | 0.691 | 4.72e−02 | 2.48e−01 |
| 43 | 1.347 | 8.58e−04 | 6.50e−02 |
| 44 | 1.815 | 2.74e−03 | 7.24e−02 |
| 45 | 0.586 | 9.58e−04 | 6.50e−02 |
| 46 | 0.702 | 1.01e−03 | 6.50e−02 |
| 47 | 0.523 | 1.11e−03 | 6.50e−02 |
| 48 | 0.734 | 1.43e−03 | 6.50e−02 |
| 49 | 0.900 | 1.47e−03 | 6.50e−02 |
| 50 | 2.432 | 2.02e−03 | 7.24e−02 |
| 51 | 1.815 | 2.74e−03 | 7.24e−02 |

The quantified abundances of various peptide structures (e.g., SEQ ID NOs: 35-42 identified in Table 9) across the entire sample set were used to train a multivariate logistic regression model to generate a disease indicator for a subject. The disease indicator was generated as a score (e.g., a probability score) in which the range in which the score falls enables diagnosis of classification as a malignant or a benign pelvic tumor state. Coefficients for the multivariate logistic models are provided in Table 13.

TABLE 13
Coefficients for each marker used in the
multivariate logistic regression models

| SEQ | Coefficients |
|---|---|
| ID NO | (Disease vs. Control) |
| 35 | −213.20 |
| 36 | −3.14 |
| 37 | −26.85 |
| 38 | 65.00 |
| 39 | 2.23 |
| 40 | 141.85 |
| 41 | −2.12 |
| 42 | −0.77 |

The relative contribution of each biomarker in this model can be correlated to the magnitude (e.g., absolute value) of each logistic regression coefficient for SEQ ID NOs: 35-42, with greater magnitudes corresponding to a greater contribution to the model's predictions. Leave one out cross-validation (LOOCV) was performed on normalized relative abundances of the biomarkers of the samples from both malignant and benign pelvic tumor patients. A logistic regression model with LASSO regularization was iteratively trained on all samples except for one sample that was left out in that iteration. The trained model was then used to predict on the sample that was left out. Table 14 provides the confusion matrix for this trained model and Table 15 provides several other performance metrics for the trained model. As shown in Table 15, the area under the receiver operating characteristic curve (AUROC) for distinguishing between the benign and malignant pelvic tumor states was found to be 1 for the training set and 0.93 for the test set. The positive predictive value (PPV) was found to be 1 for the training set and 0.75 for the test set and the negative predictive value (NPV) was found to be 1 for both of the training and test sets.

These results support the use of a model trained using the relative abundance of peptide structures for the diagnosis of malignancy for pelvic tumors and demonstrate that the glycopeptides described herein can be used to differentiate benign and malignant pelvic tumors (such as ovarian cancer) using DBS samples.

TABLE 14
Confusion matrix comparing predicted outcomes with actual
malignancy status for both training and test sets

| Predicted outcome | | |
|---|---|---|
| for train/test | True benign | True malignant |
| Benign (train) | 10 | 0 |
| Malignant (train) | 0 | 8 |
| Benign (test) | 5 | 0 |
| Malignant (test) | 0 | 3 |

TABLE 15
Performance metrics for the trained multivariate logistic
regression model for pelvic tumor malignancy diagnosis

| | AUROC | Accuracy | Sensitivity | Specificity | PPV | NPV |
|---|---|---|---|---|---|---|
| Training | 1 | 1 | 1 | 1 | 1 | 1 |
| set | | | | | | |
| Test set | 0.93 | 0.875 | 1 | 0.8 | 0.75 | 1 |

### Example 15. Digestion of Samples Prior to Enrichment and Analysis

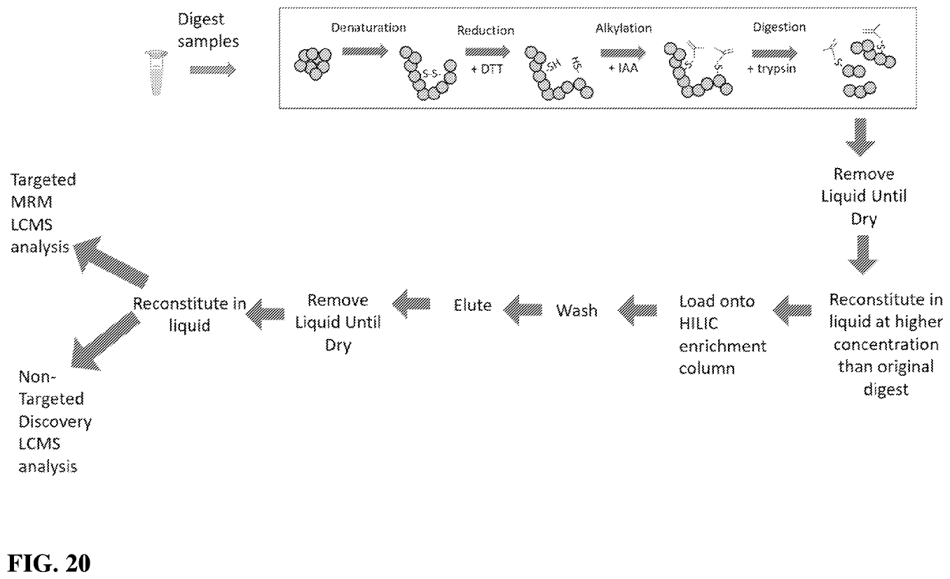

A schematic for the overall workflow for sample preparation and analysis is given in FIG. 20 for identifying new glycoproteins and glycoforms that are suitable for use as biomarkers for diagnosing a disease.

Pooled human serum for assay normalization and calibration purposes, dithiothreitol (DTT), and iodoacetamide (IAA) were purchased from Millipore Sigma (St. Louis, MO). Sequencing grade trypsin was purchased from Promega (Madison, WI). Acetonitrile (LC-MS grade) was purchased from Honeywell (Muskegon, MI). All other reagents used were procured from Millipore Sigma, VWR, and Fisher Scientific.

In the first step described for the method of this example, ammonium bicarbonate (50 mM) and dithiothreitol (DTT, 50 mM) solutions were freshly prepared. The ammonium bicarbonate solution was used to make the DTT solution. Immediately prior to transfer, each biological sample and control was gently vortexed for 10 seconds. Using a single channel pipette, 5 μL of biological sample or control (e.g., plasma or serum) was transferred into a deep-well digestion plate, wherein the plate is compatible with thermal cycling. To this, the 35 μL of 50 mM ammonium bicarbonate solution was added. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute.

The sample plate containing the sample was incubated in a thermal cycler for 5 minutes, wherein the thermal cycler was set to 100° C. with a lid temperature of 105° C. All heated plates were allowed to cool to room temperature before removing from the respective heat source and spinning at 370×g for 1 minute. After the spin, the plate seals were removed.

After protein denaturation, all samples were reduced by adding 20 μL of the 50 mM DTT solution into each sample and control well. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute. Plates were then incubated in a 60° C. water bath for 50 minutes. Plates were then removed from the water bath and centrifuged at 4,800×g for 1 minute before removing the plate seals.

Prior to the completion of this reduction incubation, a fresh 90 mM iodoacetamide (IAA) solution was prepared, and the container with the IAA solution was covered in foil. When ready, samples were alkylated by adding 20 μL of the 90 mM IAA solution into each sample and control well. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute. Plates were then incubated in the dark at room temperature for 30 minutes. After the incubation, plate seals were removed and 10 μL of the 50 mM DTT solution was added to quench any remaining IAA in solution. The plates were then sealed with a foil heat seal using a plate sealer and vortexed at 1400 RPM for 1 minute on a microplate mixer. Plates were centrifuged at 370×g for 1 minute and the plate seals were removed.

Prior to the completion of this alkylation incubation, fresh protease solutions were prepared (trypsin or trypsin/LysC combination). For example, for the trypsin solution, trypsin powder is dissolved in the 50 mM ammonium bicarbonate solution for a final concentration of 0.167 μg/μL trypsin solution. To the quenched biological samples and controls, 60 μL of the 0.167 μg/μL trypsin solution was added to each well. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute. Plates were then incubated in a 37° C. water bath for 18 hours. Plates were then removed from the water bath and centrifuged at 4,800×g for 1 minute before removing the plate seals.

20 μL of freshly prepared 9% formic acid solution was added to each well containing the proteolytic digested samples to stop the enzyme reaction and form the tryptically digested samples. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute.

### Example 16. Enrichment of Proteolytic Digest Samples

Tryptically digested samples from Example 15 were enriched for glycopeptides using a hydrophilic interaction liquid chromatography (HILIC) concentration phase. The HILIC sorbent material used in this example was the Agilent GlykoPrep Cleanup (CU) Cartridges (30 μg glycan capacity) on Agilent Bravo Platform for AssayMAP (liquid handler). This enrichment process increased the proportion of glycopeptides with respect to the peptides in the sample because of the higher affinity between the glycans and the HILIC sorbent material than peptides and the HILIC sorbent material.

For 1ss samples, 170 μL of serum digest was collected and then the liquid was removed with a SpeedVac evaporator until the sample was dry (SpeedVac was set as autorun with a vacuum pressure of 5.1 mtorr and no temperature control). The 170 μL of serum digest contains 300 μg of polypeptide content (based on a literature standard value of serum protein concentration of 60 μg/μl and the initial input of 5 μl of serum to produce the digest). Next, the dry sample was reconstituted by adding 41.8 μL of deionized water to each well of a plate and then each plate was sealed with peelable foil heat seals (BioRad) using a plat sealer (BioRad PX1 PCR Plate Sealer). After sealing, the plate was sonicated for five, 5 minutes intervals where ice was added to the sonicator in between each interval to avoid a rise in temperature. The foil was removed from the plate and 178.2 μL of 1.23% trifluoracetic acid (TFA)/Acetonitrile (ACN) was added to each well of a plate and then each plate was sealed with peelable foil heat seals. After sealing, the plate was sonicated for three, 4 minutes intervals where ice was added to the sonicator in between each interval. For 2ss, a corresponding protocol was used starting with 340 μl of serum digest containing 600 μg of polypeptide content.

The plate was centrifuged at 1300 rpm at 4° C. for 1 minute. Following centrifugation, the foil was removed and 200 μL of the reconstituted solution was removed from a well and loaded onto a dry Agilent GlykoPrep Cleanup (CU) Cartridge at a 3 μL/min flow rate. The liquid output from the cartridge during the loading process was discarded. The cartridge was washed with 200 μL of a Cartridge Wash Buffer of 1% TFA, 96% ACN in deionized water at a 3 μL/min flow rate. After washing, the cartridge was eluted with 100 μL of a Cartridge Elution Buffer of 0.1% TFA in deionized water at a 3 μL/min flow rate. The elution buffer (100 μL) outputted from the cartridge was collected and then dried with a SpeedVac evaporator to form the enriched sample. The enriched samples were stored at −20° C.

### Example 17. Mass Spectrometry of Enriched Proteolytic Digests

This example demonstrates a method for mass spectrometry analysis using the enriched samples. The dried enriched samples were reconstituted with 30 μL of 0.1% formic acid in water and then vortexed.

Once a sample was reconstituted, it was directly introduced into an LC-MS system using an LC diversion technique. All samples were subjected to the same instrument method, wherein 0-3 minutes were diverted to waste, 3-47.8 minutes were passed to the MS instrument, and 47.8-49 minutes were again diverted to waste. During this time course, a constant solvent gradient was completed. The aqueous mobile phase A was 0.1% formic acid in water (vol:vol), and the organic mobile phase B was 0.1% formic acid in acetonitrile (vol:vol). Separation of peptides and glycopeptides was performed using a binary gradient of 0.0-9.0 min, 1-10% B; 9.0-36.0 min, 10-25% B; 36.0-48.0 min, 25-44% B; 48.0-48.1 min, 44-1% B; 48.1-49.0 min, 1% B. The liquid chromatography system was an Agilent 1290 Infinity II UHPLC system that used a 20 μL loop volume, 4 μL injection volume, Waters ACQUITY UPLC Peptide HSS T3 Column, 100 Å port volume, 1.8 μm particle size, 2.1 mm×150 mm (diameter×length) with HSS T3 guard column, 2.1 mm×5 mm. The output of the chromatography column was either outputted to a waste channel or to the mass spectrometer via an electrospray ionization unit using a microprocessor-controlled valve depending on the time of the chromatography run (see Table 1).

TABLE 1
Chromatography control parameters.

| Time | Scan Type | Divert vale |
|---|---|---|
| 0-3 minutes | dMRM | To waste |
| 3-47.8 minutes | dMRM | To MS |
| 47.8-49 minutes | dMRM | To waste |

FIG. 3A shows a valve position 1 where the chromatography column output goes to waste via ports 2 and 3 of the diverter valve and FIG. 3B shows a valve position 2 where the chromatography output goes to waste via ports 2 and 3 of the diverter valve. The mass spectrometry system was an Agilent 6495C triple quadrupole mass spectrometer. Samples were introduced into the mass spectrometer using an electrospray ionization (ESI) source operated in the positive ion mode. Nitrogen drying and sheath gas temperatures were set at 290° C. and 300° C., respectively. Drying and sheath gas flow rates were set at 11 L/min and 12 L/min, respectively. The nebulizer pressure was set to 30 psi. Data acquired from the UHPLC/QqQ-MS was collected using Agilent MassHunter Workstation LC/MS Data Acquisition B10.1.67. Sample analysis was performed using a dynamic multiple reaction monitoring (dMRM) method. Collision induced dissociation was used for fragmentation.

### Example 18. Mass Spectrometry of Enriched Proteolytic Digests with Varying Sample Loading

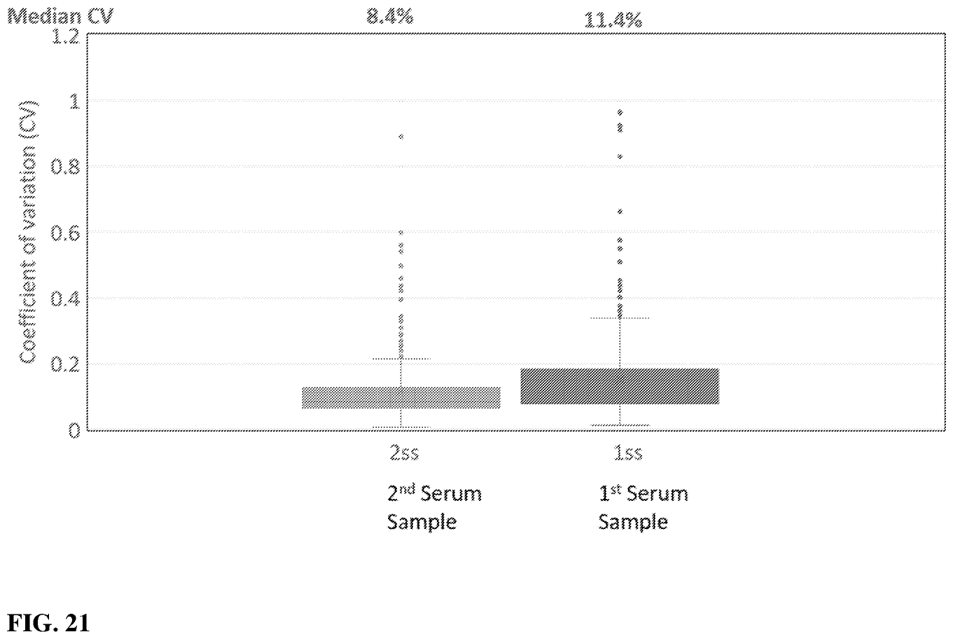

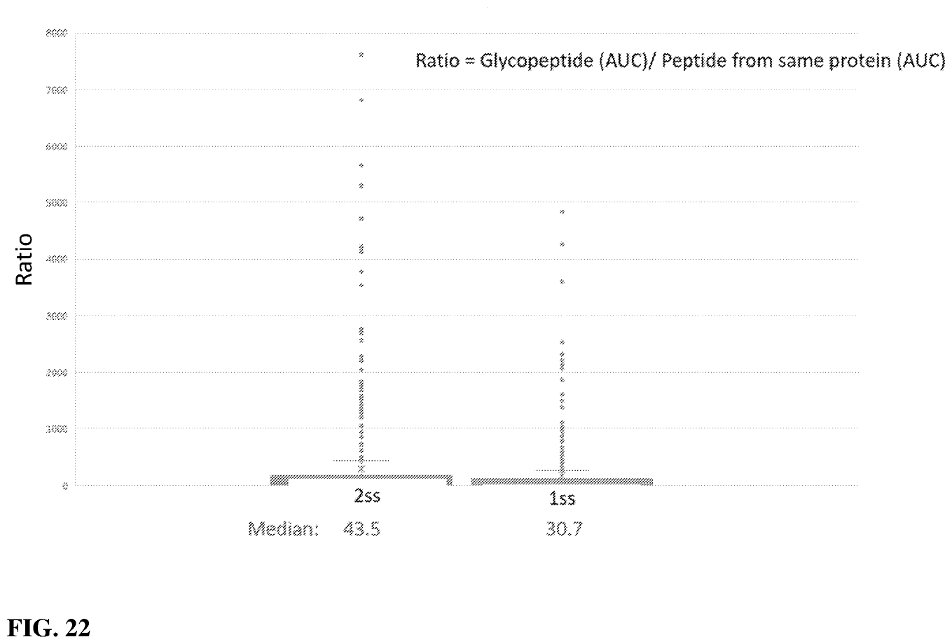

Serum samples were digested using the method of Example 15. The protein amount was varied so that either 300 μg (1ss) or 600 μg (2ss) of serum protein was enriched using the method of Example 16 to form enriched samples. 170 μl of serum digest provides the 1ss aliquot, and 340 μl of serum digest provides the 2ss aliquot. The two serum protein samples were tested with a MRM-MS method in accordance with Example 17. As illustrated in FIG. 21, the median CV values observed for the 1ss and 2ss samples were 11.4% and 8.4%, respectively. For this MRM-MS method, a panel of more than 500 glycopeptides were measured with 4 replicates. The higher 600 μg loading (2ss) was found to show an improved level of precision compared to the lower 300 μg loading (1ss). In addition, a ratio was calculated of the peak area of a glycopeptide divided by a peak area of a peptide that was attached to the same protein. As illustrated in FIG. 22, the median ratio was 30.7 and 43.5 for the 1ss and 2ss samples, respectively, indicating a significant enrichment of glycopeptides with respect to unglycosylated peptides.

### Example 19. Mass Spectrometry of Enriched Proteolytic Digests with Loading Solutions Having Different % of ACN

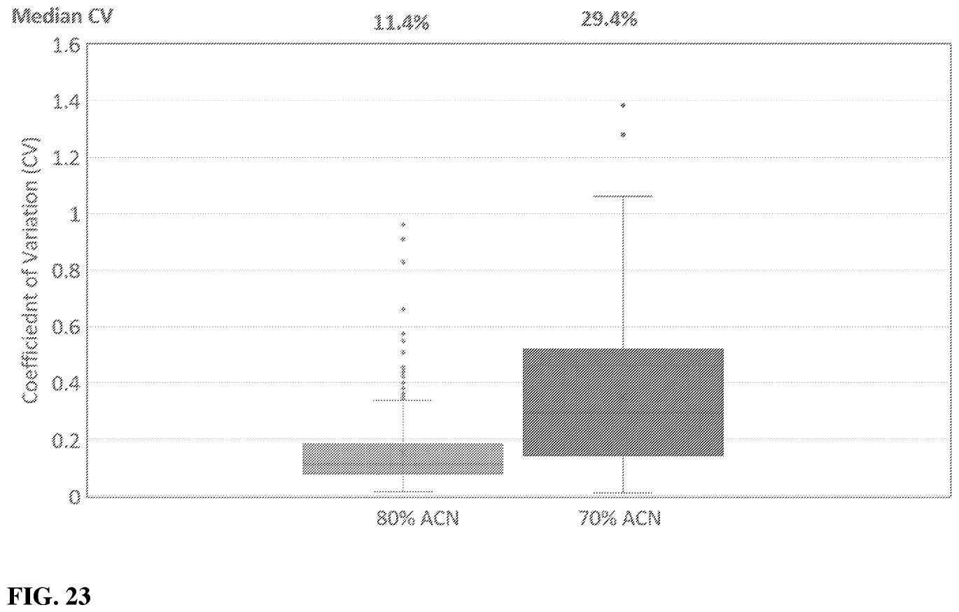

Serum samples were digested using the method of Example 15. In this example, the protein amount was selected to be 300 μg (1ss) serum protein. The samples were enriched in a manner similar to Example 16 where one sample was loaded onto the HILIC material with a 1% TFA, 80% ACN loading solution and the other sample was loaded onto the HILIC material with a 1% TFA, 70% ACN loading solution. The two sample types were tested in a LC-MS method in accordance with Example 17. As illustrated in FIG. 23, the median CV values observed for the 1% TFA, 80% ACN and 1% TFA, 70% ACN were 11.4% and 29.4%, respectively. The higher 80% ACN loading solution was found to show an improved level of precision compared to the lower 70% ACN loading solution.

### Example 20. Mass Spectrometry of Enriched Proteolytic Digests with and without HILIC Enrichment for Measuring a Glycopeptide

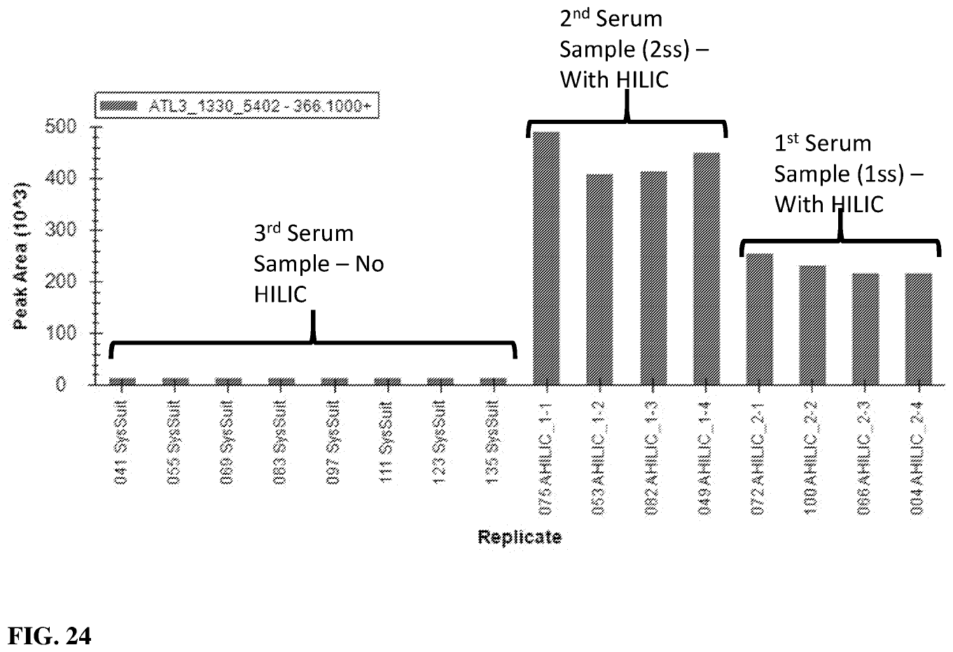

Serum samples were digested using the method of Example 15. A first and second serum protein sample of 300 μg (1ss) and 600 μg (2ss), respectively, were enriched using the method of Example 16 to form enriched samples. In this Example, a third sample was prepared with 300 μg of serum protein from Example 15, but no HILIC enrichment step was performed. The three serum protein samples were tested with a LC-MS method in accordance with Example 17. As illustrated in FIG. 24, the glycopeptide (ATL3_1330_5402-366.1000+) was measured with 4 replicates for the first and second serum sample with an average peak area of 444,797.3 (2ss) and 232,503.6 (1ss). In contrast, the glycopeptide (ATL3_1330_5402-366.1000+) was measured with 8 replicates for the third serum sample with an average peak area of 11,801.17. It should be noted that each replicate along the x-axis of FIG. 24 has a unique name for tracking purposes. The higher peak area for the first and the second serum sample showed that HILIC enrichment for a glycopeptide provided a significantly higher peak area compared to the peak area measured for the third serum sample with no HILIC enrichment. It should be noted that ATL3_1330_5402-366.1000+ represents a glycopeptide from the protein name ADAMTS-likeProtein3 (Unitprot ID P82987) where the glycan is attached at linking site position with the protein sequence of 1330. The glycopeptide ATL3_1330_5402-366.1000+ has a peptide sequence of GVPQPNITWLKR and the glycan is attached at linking site position with the peptide sequence of 6. The product ion from the glycopeptide ATL3_1330_5402-366.1000+ has a m/z ratio of 366.1. The glycopeptide ATL3_1330_5402-366.1000+ has a monoisotopic mass of 3612.571028, a collision energy of 36 V, a retention time of 21.4 minutes, and a composition of Hex(5)HexNAc(4)Fuc(0)NeuAc(2). The terms Hex, HexNAc, Fuc, and NeuAc respectively correspond to hexose, N-acetylhexosamine, fucose, and N-acetylneuraminic acid.

### Example 21. Mass Spectrometry of Enriched Proteolytic Digests with and without HILIC Enrichment for Measuring an Unglycosylated Peptide

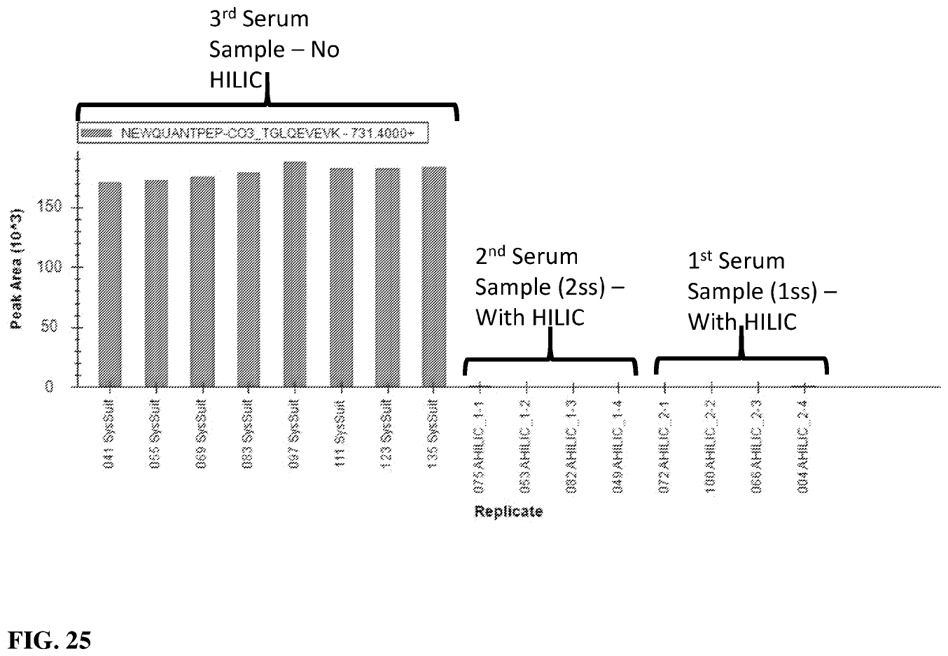

Serum samples were digested using the method of Example 15. A first and second serum protein sample of 300 μg (1ss) and 600 μg (2ss), respectively, were enriched using the method of Example 16 to form enriched samples. In this Example, a third sample was prepared with 300 μg of serum protein from Example 15, but no HILIC enrichment step was performed. The three serum protein samples were tested with a LC-MS method in accordance with Example 17. As illustrated in FIG. 25, the unglycosylated peptide TGLQEVENVK was measured with 4 replicates for the first and second serum sample with a relatively low average peak area of 891.8 and 786.5. In contrast, the unglycosylated peptide TGLQEVENVK was measured with 8 replicates for the third serum sample with a relatively high average peak area of 178,759.7. It should be noted that each replicate along the x-axis of FIG. 25 has a unique name for tracking purposes. The higher peak area for the third serum sample showed that the HILIC enrichment did not concentrate unglycosylated peptides suggesting that glycopeptides were preferentially bound to the HILIC stationary phase with respect to unglycosylated peptides.

### Example 22. Methods of Defibrination and Digestion of Plasma Samples

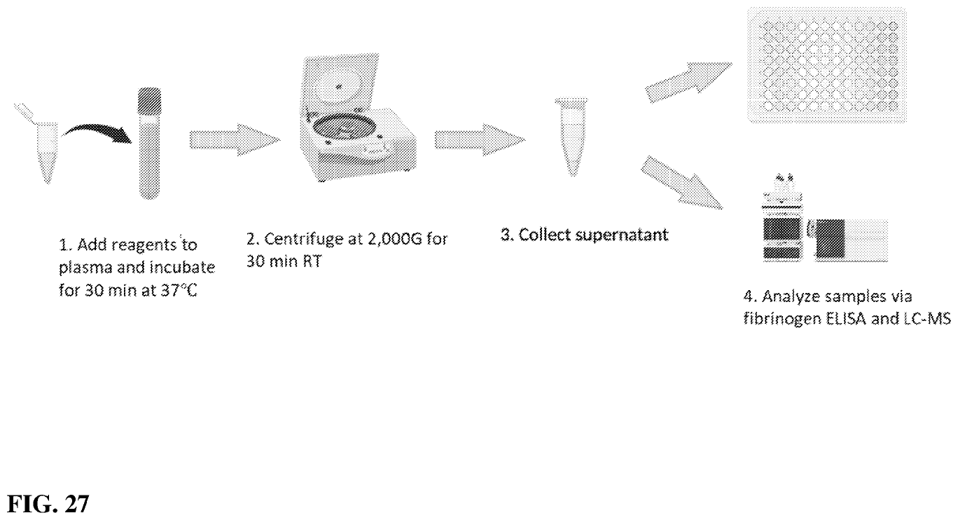

A schematic for the overall workflow for defibrination of plasma samples is given in FIG. 27. Plasma samples were first dosed with defibrination reagents. Prior to treatment, each plasma sample was about 40 μL in volume. The defibrination reagents comprised CaCl₂, thrombin, Kaolin clay, and combinations thereof. CaCl₂) was added to plasma samples to yield a final concentration between 10 and 20 mM of CaCl₂), including 10 mM, 15 mM, and 20 mM. Likewise, thrombin was added to plasma samples to yield a final concentration of 5 units/mL. Kaolin clay was added to plasma samples as a dose of between 100-400 μg of Kaolin clay per sample. After the addition of defibrination agents, plasma samples were incubated at 37° C. for 30 minutes. Following incubation, the plasma samples were centrifuged at 2,000 G for 30 minutes at room temperature. The supernatant of each plasma sample was collected after centrifugation. For samples that received a dose of the Kaolin clay, the compacted clot layer was easier to visually distinguish and the supernatant was notably easier to collect. The resulting defibrinated plasma samples were then assessed via a fibrinogen ELISA assay (Example 23) or with LC-MS (Example 24) after digestion.

Prior to any LC-MS analysis, the defibrinated plasma samples were subjected to tryptic digestion. In the first step described for the tryptic digestion method herein, ammonium bicarbonate (50 mM) and dithiothreitol (DTT) (50 mM) solutions were freshly prepared. The ammonium bicarbonate solution was used to make the DTT solution. Pre-thawed biological samples and controls were inspected for turbidity, hemolysis, clotting, and precipitation. Immediately prior to transfer, each biological sample and control was gently vortexed for 10 seconds. Using a single channel pipette, 5 μL of biological sample or control (e.g., plasma or serum) was transferred into a deep-well digestion plate, wherein the plate is compatible with thermal cycling. To this, the 35 μL of 50 mM ammonium bicarbonate solution was added. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute.

A separate sample plate was incubated in a thermal cycler for 5 minutes, wherein the thermal cycler was set to 100° C. with a lid temperature of 105° C. All heated plates were allowed to cool to room temperature before removing from the respective heat source and spinning at 370×g for 1 minute. After the spin, the plate seals were removed.

After protein denaturation, all samples were reduced by adding 20 μL of the 50 mM DTT solution into each sample and control well. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute. Plates were then incubated in a 60° C. water bath for 50 minutes. Plates were then removed from the water bath and centrifuged at 4,800×g for 1 minute before removing the plate seals.

Prior to the completion of this reduction incubation, a fresh 90 mM iodoacetamide (IAA) solution was prepared, and the container with the IAA solution was covered in foil. When ready, samples were alkylated by adding 20 μL of the 90 mM IAA solution into each sample and control well. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute. Plates were then incubated in the dark at room temperature for 30 minutes. After the incubation, plate seals were removed and 10 μL of the 50 mM DTT solution was added to quench any remaining IAA in solution. The plates were then sealed with a foil heat seal using a plate sealer and vortexed at 1400 RPM for 1 minute on a microplate mixer. Plates were centrifuged at 370×g for 1 minute and the plate seals were removed.

Prior to the completion of this alkylation incubation, fresh protease solutions were prepared (trypsin or trypsin/LysC combination). For example, for the trypsin solution, trypsin powder is dissolved in the 50 mM ammonium bicarbonate solution for a final concentration of 0.167 μg/μL trypsin solution. To the quenched biological samples and controls, 60 μL of the 0.167 μg/μL trypsin solution was added to each well. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute. Plates were then incubated in a 37° C. water bath for 18 hours. Plates were then removed from the water bath and centrifuged at 4,800×g for 1 minute before removing the plate seals.

20 μL of freshly prepared 9% formic acid solution was added to each well containing the proteolytic digested samples to stop the enzyme reaction. The plates were then sealed with a foil heat seal using a plate sealer. To ensure all samples were mixed thoroughly, the plates were vortexed at 1400 RPM for 1 minute on a microplate mixer, followed by centrifugation at 370×g for 1 minute.

### Example 23. ELISA Assays of Defibrinated Plasma Samples

Na-citrated plasma samples were defibrinated according to the defibrination procedure outlined in Example 22. Several defibrinated plasma samples were prepared according to the following formulations of defibrination reagents: (1) 10 mM CaCl₂) (Ca); (2) 15 mM CaCl₂); (3) 20 mM CaCl₂); (4) 20 mM CaCl₂) in combination with 100 μg of Kaolin clay (K); (5) 20 mM CaCl₂) in combination with 200 μg of Kaolin clay; (6) 20 mM CaCl₂) in combination with 5 units/mL thrombin (Thr); (7) 20 mM CaCl₂) in combination with 5 units/mL thrombin and 100 μg of Kaolin clay; and (8) 20 mM CaCl₂) in combination with 5 units/mL thrombin and 200 μg of Kaolin clay. Mock treated plasma and mock treated serum samples were produced by treating plasma and serum samples with deionized and filtered MilliQ water rather than being treated with any defibrination reagents to account for the volume change in comparison with treated samples.

The human fibrinogen ELISA kit used for analysis had a reported sensitivity of 29 μg/mL of fibrinogen with a range of 125-8000 μg/mL of fibrinogen. Mock treated plasma samples were diluted at a factor of 106 prior to the assay and treated plasma samples and mock treated serum samples were diluted at a factor of 2000 prior to the assay. Samples were loaded onto 96-well plates with 50 μL of sample per well and the ELISA assay was conducted to determine a concentration of fibrinogen for each sample. Mock treated plasma and mock treated serum were found to have comparable fibrinogen content as that of untreated plasma and serum samples, respectively, verifying that the experimental process does not interfere with native fibrinogen concentration. Average serum concentration of fibrinogen has been reported as 3.45 μg/mL and the reference serum concentration was determined to be around 1 μg/mL of fibrinogen. Mock treated plasma fibrinogen concentration was determined to be around 3.86 mg/mL.

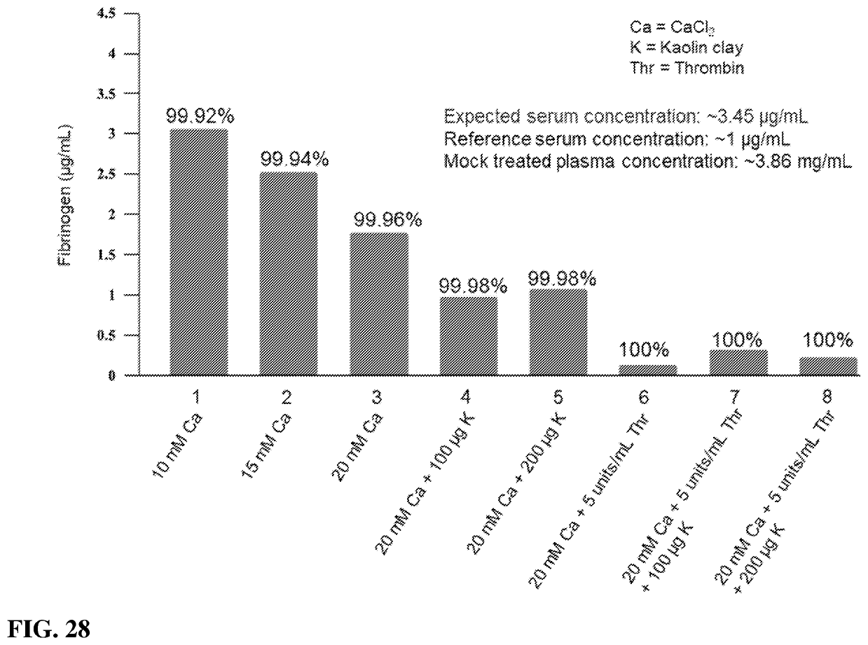

The results of the human fibrinogen ELISA assay of the defibrinated plasma samples are presented in FIG. 28. The absolute concentration of fibrinogen in the treated samples can be determined via the value of each bar plot in FIG. 28 and the relative decrease in fibrinogen content compared to mock treated plasma is listed above each treated sample's respective bar plot in FIG. 28. Each of the treated samples were determined to have 3 μg/mL or less of fibrinogen content, which corresponds to 99.92-100% removal of fibrinogen compared to mock treated plasma. CaCl₂) was found to be effective at defibrination in the range of 10-20 mM. Treatment with thrombin (Thr) yielded visual clotting of the plasma samples within 1 minute after treatment at room temperature. Although each treatment removed at least 99.92% of fibrinogen when compared to mock treated plasma, a combination of more than one reagent was found to be effective treatment for fibrinogen removal.

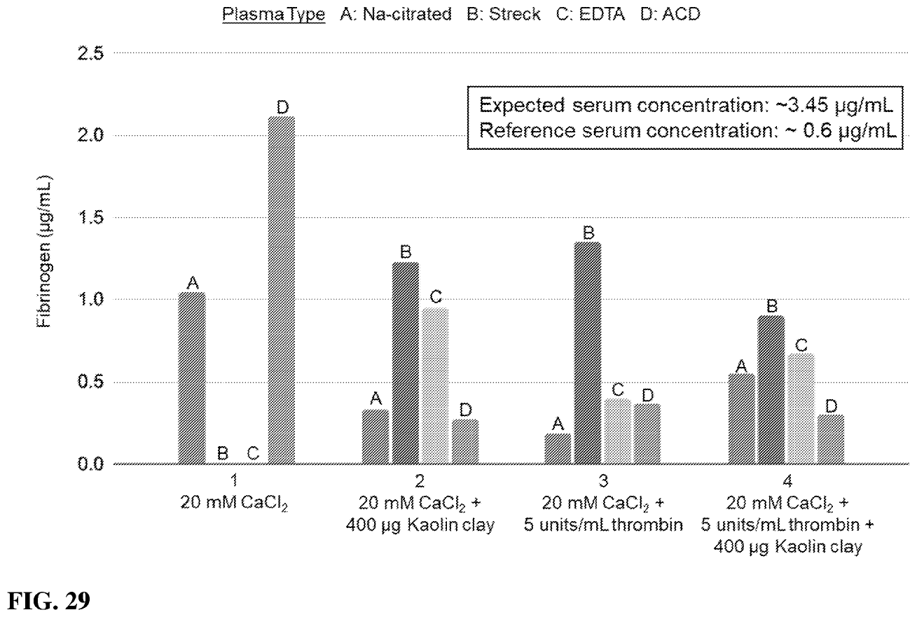

To test the efficacy of the defibrination treatment for other types of plasma than Na-citrated plasma, the human fibrinogen ELISA assay was also conducted using Streck, EDTA, and ACD plasmas. The (A) Na-citrated, (B) Streck, (C) EDTA, and (D) ACD plasmas were each defibrinated according to the defibrination procedure outlined in Example 22. Several defibrinated plasma samples were prepared according to the following formulations of defibrination reagents: (1) 20 mM CaCl₂); (2) 20 mM CaCl₂) and 400 μg of Kaolin clay; (3) 20 mM CaCl₂ and 5 units/mL thrombin; and (4) 20 mM CaCl₂) with 5 units/mL thrombin and 400 μg of Kaolin clay. Mock treated plasma and mock treated serum were prepared as in the above Na-citrated plasma ELISA study and samples were also prepared and loaded onto 96-well plates in the same fashion. The results of the ELISA assay for multiple plasma types are presented in FIG. 29. Reference serum fibrinogen concentration for this assay was determined to be around 0.6 μg/mL. The fibrinogen concentration for each treated plasma type was determined to be under 2.5 μg/mL for each of the defibrination formulation treatments, verifying the maintained performance of the defibrination procedure for various plasma types.

### Example 24. LC-MS Analysis of Defibrinated and Digested Plasma Samples

Na-citrated plasma samples were defibrinated and digested in preparation of LC-MS analysis according to the defibrination and digestion procedures outlined in Example 22. Plasma samples were treated with CaCl₂) (C), Kaolin clay (K), thrombin (T), and combinations thereof (e.g., “C-T-K” refers to treatment with a formulation of CaCl₂, thrombin, and Kaolin clay, etc.). Four treated sample types were prepared for this study: C-T-K plasma, containing 20 mM CaCl₂, 5 units/mL thrombin, and 200 μg Kaolin clay; C-K plasma, containing 20 mM CaCl₂ and 100 μg Kaolin clay; C plasma, containing 20 mM CaCl₂); and T plasma, containing 10 units/mL thrombin. Mock treated plasma and mock treated serum samples were produced by treating plasma and serum with deionized and filtered MilliQ water rather than being treated with any defibrination reagents.

All samples were subjected to the same LC-MS instrument method, wherein 0-3 minutes were diverted to waste, 3-47.8 minutes were passed to the MS instrument, and 47.8-49 minutes were again diverted to waste. During this time course, a constant solvent gradient was completed. The aqueous mobile phase A was 0.1% formic acid in water (vol:vol), and the organic mobile phase B was 0.1% formic acid in acetonitrile (vol:vol). Separation of peptides and glycopeptides was performed using a binary gradient of 0.0-9.0 min, 1-10% B; 9.0-36.0 min, 10-25% B; 36.0-48.0 min, 25-44% B; 48.0-48.1 min, 44-1% B; 48.1-49.0 min, 1% B. The liquid chromatography system was an Agilent 1290 Infinity II UHPLC system that used a 20 μL loop volume, 4 μL injection volume, Waters ACQUITY UPLC Peptide HSS T3 Column, 100 Å port volume, 1.8 μm particle size, 2.1 mm×150 mm (diameter×length) with HSS T3 guard column, 2.1 mm×5 mm. The output of the chromatography column was either outputted to a waste channel or to the mass spectrometer via an electrospray ionization unit using a microprocessor controlled valve depending on the time of the chromatography run (see Table 1).

TABLE 1
Chromatography control parameters.

| Time | Scan Type | Divert vale |
|---|---|---|
| 0-3 minutes | dMRM | To waste |
| 3-47.8 minutes | dMRM | To MS |
| 47.8-49 minutes | dMRM | To waste |

FIG. 3A shows a valve position 1 where the chromatography column output goes to waste via ports 2 and 3 of the diverter valve and FIG. 3B shows a valve position 2 where the chromatography output goes to waste via ports 2 and 3 of the diverter valve. The mass spectrometry system was an Agilent 6495C triple quadrupole mass spectrometer. Samples were introduced into the mass spectrometer using an electrospray ionization (ESI) source operated in the positive ion mode. Nitrogen drying and sheath gas temperatures were set at 290° C. and 300° C., respectively. Drying and sheath gas flow rates were set at 11 L/min and 12 L/min, respectively. The nebulizer pressure was set to 30 psi. Data acquired from the UHPLC/QqQ-MS was collected using Agilent MassHunter Workstation LC/MS Data Acquisition B10.1.67. Sample analysis was performed using a dynamic multiple reaction monitoring (dMRM) method. Collision induced dissociation was used for fragmentation.

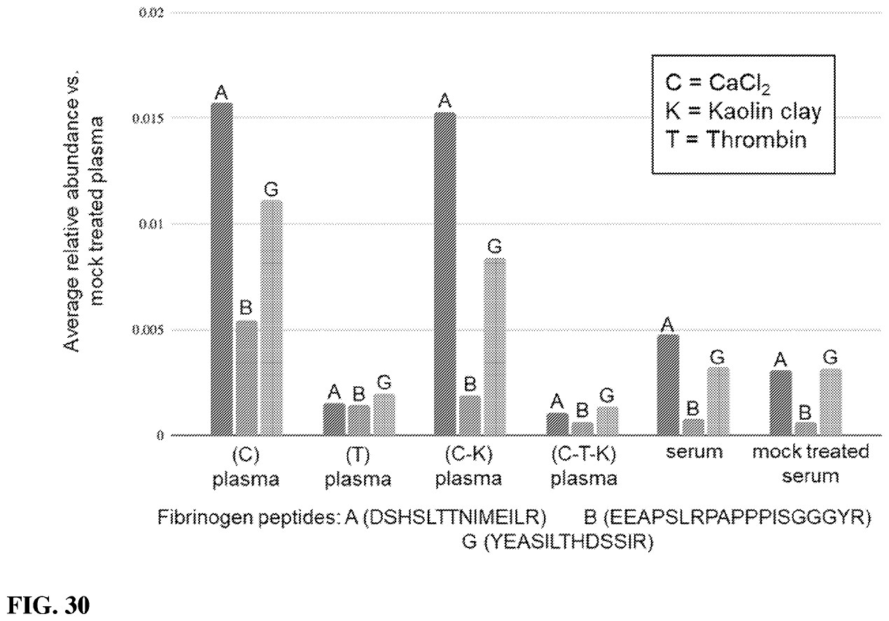

The average abundances of three fibrinogen peptides were calculated for each cohort of samples that received the same treatment relative to average abundance of the three fibrinogen peptides detected in mock treated plasma. The three analyzed fibrinogen peptides A, B, and G comprise the following amino acid sequences, respectively: DSHSLTTNIMEILR, EEAPSLRPAPPPISGGGYR, and YEASILTHDSSIR. The results of the LC-MS quantification of relative abundance for these three fibrinogen peptides are presented in FIG. 30. As seen in FIG. 30, treated plasma samples, serum samples, and mock serum samples all were found to have relative abundance of fibrinogen peptides around 0.015 and below compared to mock treated plasma.

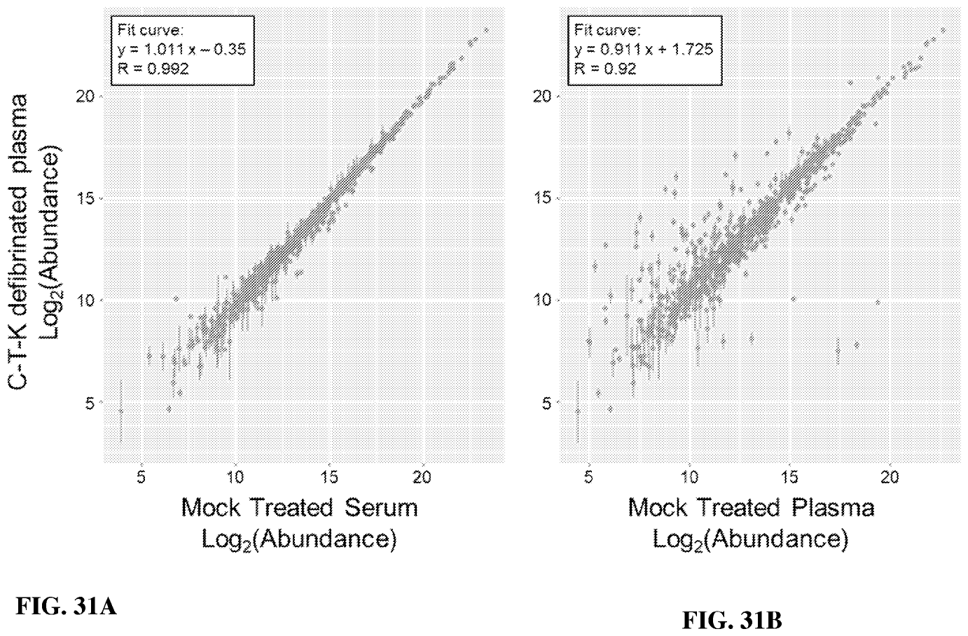

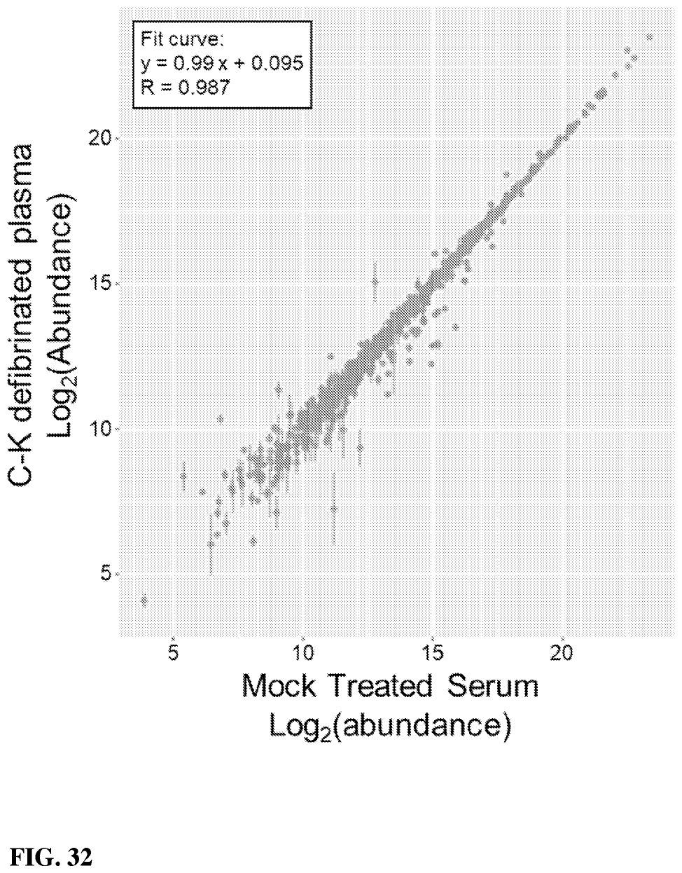

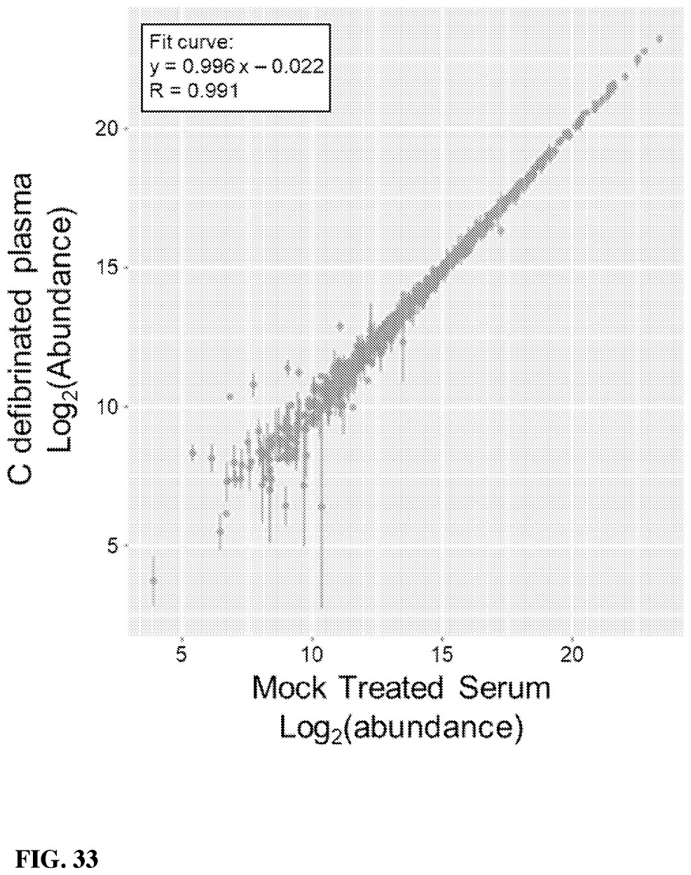

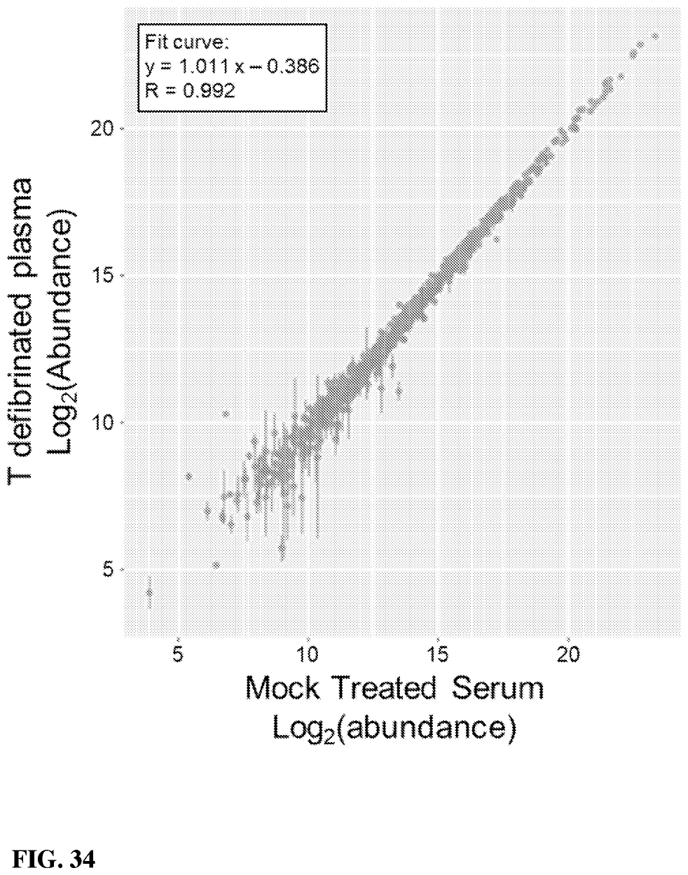

To demonstrate the effect defibrination has on plasma samples, the abundance of a plurality of peptide structures was measured in the samples using LC-MS and the log₂ (abundance) for each of the plurality of peptide structures was plotted between sample treatment types. The correlation between sample treatment types of the log₂ (abundance) for the plurality of peptide structures is presented in FIGS. 8-11 for various pairs of sample treatment types. As shown in FIGS. 31A and 31B, there is a higher Pearson's R correlation and tighter clustering between C-T-K defibrinated plasma and mock treated serum (R=0.992) than between C-T-K defibrinated plasma and mock treated plasma (R=0.92). In addition, the scatterplot of the C-T-K defibrinated plasma and mock treated serum showed a slope of one whereas the scatterplot of the C-T-K defibrinated plasma and mock treated plasma showed a slope of 0.91. Accordingly, defibrinated plasma is more closely correlated with serum than plasma. A similar performance is depicted for C-K defibrinated plasma in FIG. 32 (R=0.987), C defibrinated plasma in FIG. 33 (R=0.991), and T defibrinated plasma in FIG. 34 (R=0.992) when each is plotted against mock treated serum.

### Example 25: Defibrination of Plasma Samples Using Silica

Plasma samples were first dosed with defibrination reagents. Prior to treatment, each plasma sample was about 40 μL in volume. The defibrination reagents comprised CaCl₂, thrombin, silica particles, and combinations thereof. Prior to other defibrination reagents being added to the plasma samples, silica particles were dosed into the plasma samples to yield a final concentration of 1 mg/mL. The silica particles had a particle size of less than 150 μm and a pore size of 12 nm with hexagonal pore morphology. After the addition of silica particles, samples were incubated at 37° C. for 30 minutes with mild shaking. Following the incubation with silica, samples were brought to room temperature and additional defibrination reagents were added. CaCl₂) was added to plasma samples to yield a final concentration of 20 mM of CaCl₂). Likewise, when present, thrombin was added to plasma samples to yield a final concentration of 2 units/mL. After the addition of all defibrination agents, plasma samples were incubated at 37° C. for 30 minutes. Following incubation, the plasma samples were centrifuged at 2,000 G for 30 minutes at room temperature. The supernatant of each plasma sample was collected after centrifugation. For samples that received a dose of the silica particles, the compacted clot layer was easier to visually distinguish and the supernatant was notably easier to collect. The resulting defibrinated plasma samples were then assessed via a fibrinogen ELISA assay.

Na-citrated, Streck, K2-EDTA, and ACD plasma samples were defibrinated according to the defibrination procedure outlined in this Example above. Each type of plasma received two defibrination treatments to yield two defibrinated plasma sample types: (1) 20 mM CaCl₂) in combination with 1 mg/mL silica particles; and (2) 20 mM CaCl₂) in combination with 2 units/mL thrombin and 1 mg/mL silica particles. Mock treated plasma and mock treated serum samples were produced by treating plasma and serum samples with deionized and filtered MilliQ water rather than being treated with any defibrination reagents to account for the volume change in comparison with treated samples.

The human fibrinogen ELISA kit used for analysis had a reported sensitivity of 29 μg/mL of fibrinogen with a range of 125-8000 μg/mL of fibrinogen. Mock treated plasma samples were diluted at a factor of 106 prior to the assay and treated plasma samples and mock treated serum samples were diluted at a factor of 2000 prior to the assay. Samples were loaded onto 96-well plates with 50 μL of sample per well and the ELISA assay was conducted to determine a concentration of fibrinogen for each sample. Mock treated plasma and mock treated serum were found to have comparable fibrinogen content as that of untreated plasma and serum samples, respectively, verifying that the experimental process does not interfere with native fibrinogen concentration. Average serum concentration of fibrinogen has been reported as 3.45 μg/mL and the reference serum concentration was determined to be around 1 μg/mL of fibrinogen. Mock treated plasma fibrinogen concentration was determined to be 3.203 mg/mL.

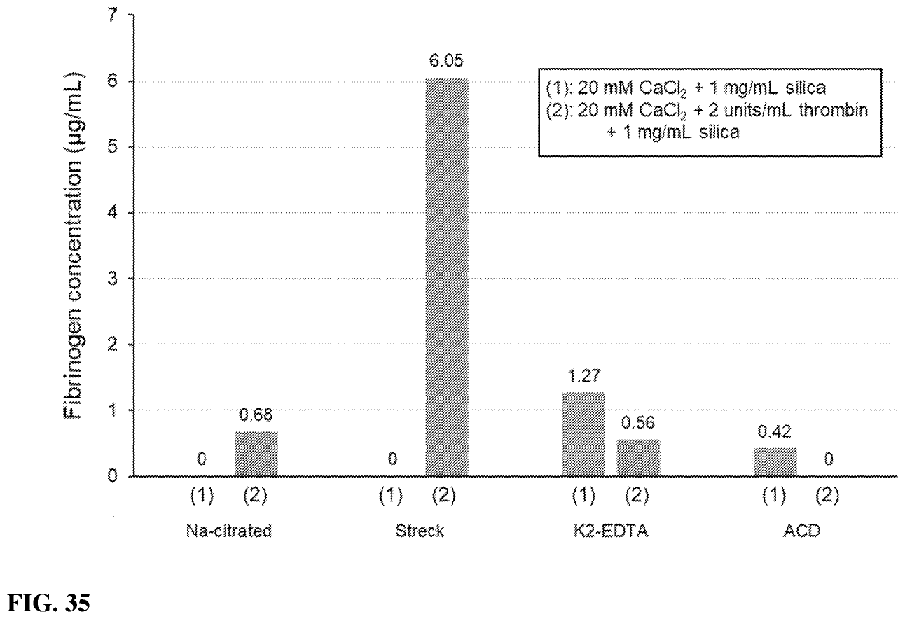

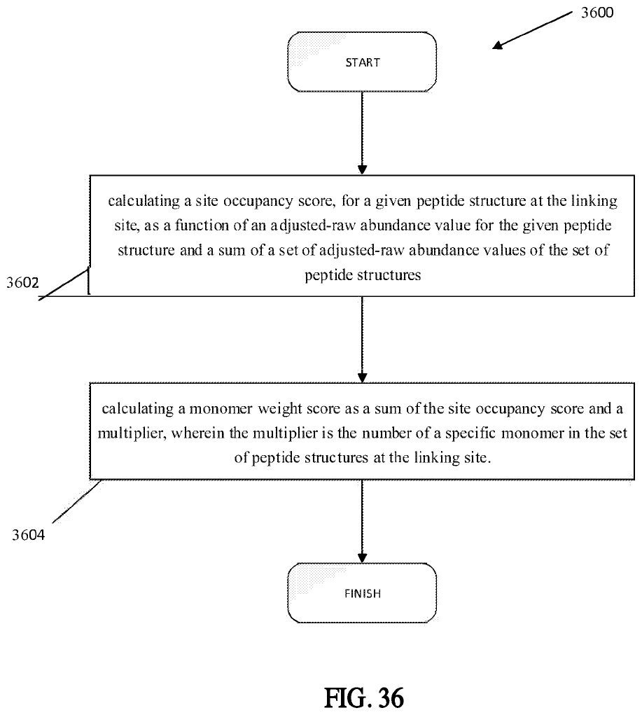

The results of the human fibrinogen ELISA assay of the defibrinated plasma samples are presented in FIG. 35. The absolute concentration in μg/mL of fibrinogen in the treated samples is provided above each bar plot in FIG. 35. A majority of the treated samples were determined to have 1.27 μg/mL or less of fibrinogen content with one outlier of 6.05 μg/mL, each of which corresponds to greater than 99% removal of fibrinogen compared to mock treated plasma.

### Example 26: Additional Methods of Defibrination of Plasma Samples

40 μL plasma samples are obtained as described in Example 22. Plasma samples are then admixed with defibrination reagents comprising one or more monovalent cations, e.g., Na⁺ or K⁺. Specifically, the defibrination reagents include combinations of one or more monovalent cation, such as Na⁺ and/or K⁺, including at concentrations of 0.2 M-0.5 M, thrombin, Kaolin clay and/or silica particles. When relevant, thrombin is added to each plasma sample to yield a final concentration of 1 unit/mL to 10 unit/mL. When relevant, Kaolin clay is added to each plasma sample at a dose of between 100-400 μg. When relevant, silica particles are added to each plasma sample to yield a final concentration of 1 mg/mL. When relevant, the addition of silica particles is the first reagent added to the plasma sample followed by incubation at 37° C. for 30 minutes with mild shaking prior to the addition of any other defibrination reagent, as specified in Example 25. After the addition of all defibrination agents, plasma samples are incubated at 37° C. for 30 minutes. Following incubation, the plasma samples are centrifuged at 2,000 G for 30 minutes at room temperature. The supernatant of each plasma sample is collected after centrifugation. After digestion and processing (as described in Example 22), the resulting defibrinated plasma samples are then assessed via a fibrinogen ELISA assay (such as described in Examples 2 or 4) or with LC-MS (such as described in Example 24).

### Additional Exemplary Embodiments

### Section 1—Proteolytic Digestion and Liquid Chromatography-Mass Spectrometry Analysis Techniques for Samples Containing a Glycosylated Polypeptide

1. A method for performing a liquid chromatography-mass spectrometry analysis of a proteolytic glycopeptide derived from a biological sample comprising a glycoprotein, the method comprising:
subjecting the biological sample to a thermal denaturation technique to produce a denatured sample followed by a proteolytic digestion technique to produce a proteolytically digested sample comprising the glycopeptide,
wherein the thermal denaturation technique subjects the biological sample to a thermal cycle comprising a thermal treatment of about 60° C. to about 100° C. with a hold time of at least about 1 minute,
wherein the lid temperature during the thermal cycle is at least about 2° C. higher than the temperature of the block temperature during the thermal cycle,
wherein the proteolytic digestion technique comprises adding an amount of one or more proteolytic enzymes and incubating for a digestion incubation time, and
wherein the digestion technique comprises quenching the one or more proteolytic enzymes following the digestion incubation time;
introducing the proteolytically digested sample to a liquid chromatography (LC) system of a LC-MS system; and
performing a LC separation to introduce the proteolytic glycopeptide to a mass spectrometer (MS) system,
wherein the LC separation comprises a period of diversion of an initial eluate comprising a salt, and
wherein the LC system comprises a reversed-phase chromatography column.
2. The method of claim 1, further comprising subjecting the denatured sample to a reduction technique followed by an alkylation technique prior to the proteolytic digestion technique.
3. The method of claim 2, wherein the reduction technique comprises subjecting the denatured sample to a reduction technique to produce a reduced sample,
wherein the reduction technique comprises adding an amount of a reducing agent to the denatured sample and incubating for a reducing incubation time.
4. The method of claim 2 or 3, wherein the alkylation technique comprises subjecting the reduced sample to an alkylation technique to produce an alkylated sample,
wherein the alkylation technique comprises adding an amount of an alkylating agent to the reduced sample and incubating substantially in in a low light condition for an alkylation incubation time, and
wherein the alkylated technique comprises quenching the alkylating agent following the alkylation incubation time.
5. A method for proteolytically digesting a biological sample comprising a glycoprotein to produce a proteolytic glycopeptide, the method comprising:
subjecting the biological sample to a thermal denaturation technique to produce a denatured sample,
wherein the thermal denaturation technique comprises subjecting the biological sample to a thermal cycle comprising a thermal treatment of about 60° C. to about 100° C. with a hold time of at least about 1 minute,
wherein the lid temperature during the thermal cycle is at least about 2° C. higher than the temperature of the block temperature during the thermal cycle;
subjecting the denatured sample to a reduction technique to produce a reduced sample,
wherein the reduction technique comprises adding an amount of a reducing agent to the denatured sample and incubating for a reducing incubation time;
subjecting the reduced sample to an alkylation technique to produce an alkylated sample,
wherein the alkylation technique comprises adding an amount of an alkylating agent to the reduced sample and incubating substantially in the dark or in a low light condition for an alkylation incubation time, and
wherein the alkylated technique comprises quenching the alkylating agent following the alkylation incubation time; and
subjecting the alkylated sample to a proteolytic digestion technique to produce a proteolytically digested sample comprising the proteolytic glycopeptide,
wherein the proteolytic digestion technique comprises adding an amount of one or more proteolytic enzymes and incubating for a digestion incubation time, and
wherein the proteolytic digestion technique comprises quenching the one or more proteolytic enzymes following the digestion incubation time.
6. The method of any one of claims 1-5, wherein the glycopeptide comprises a hydrophilic glycan portion.
7. The method of any one of claims 1-5, wherein the glycopeptide comprises a hydrophobic glycan portion.
8. The method of any one of claims 1-7, wherein the biological sample is derived from a human.
9. The method of any one of claims 1-8, wherein the biological sample is a blood sample or a derivative thereof.
10. The method of any one of claims 1-9, wherein the biological sample is a plasma sample.
11. The method of any one of claims 1-10, wherein the biological sample is a serum sample.
12. The method of any one of claims 1-11, wherein the biological sample is not subjected to a high-abundant protein depletion technique prior to the thermal denaturation technique.
13. The method of any one of claims 1-12, wherein the thermal cycle comprises a block set temperature of about 60° C. to about 100° C. with a hold time of at least about 1 minute.
14. The method of any one of claims 1-13, wherein the thermal cycle comprises a block ending temperature of about 15° C. to about 40° C.
15. The method of any one of claims 1-14, wherein the thermal cycle comprises a block starting temperature of about 15° C. to about 50° C.
16. The method of any one of claims 1-15, wherein the thermal cycle is performed in a thermal cycler comprising a lid temperature control element.
17. The method of any one of claims 1-16, wherein the thermal cycle comprises a ramp rate between the block set temperature and the block ending temperature of about 1° C./second to about 10° C./second.
18. The method of any one of claims 1-17, wherein the proteolytic digestion technique is performed at a temperature of about 20° C. to about 55° C.
19. The method of any one of claims 1-18, wherein the digestion incubation time is at least about 20 minutes.
20. The method of any one of claims 1-19, wherein the proteolytic digestion technique is performed at a temperature of about 37° C. for at least about 12 hours.
21. The method of any one of claims 18-20, wherein the proteolytic digestion technique is performed using a second thermal cycle, wherein the lid temperature during the second thermal cycle is at least about 2° C. higher than the temperature of the block temperature during the second thermal cycle.
22. The method of claim 21, wherein the second thermal cycle is performed in a thermal cycler comprising a lid temperature control element.
23. The method of any one of claims 1-22, wherein each of the one or more proteolytic enzymes is selected from the group consisting of trypsin and LysC.
24. The method of claim 23, wherein the trypsin is methylated and/or acetylated.
25. The method of any one of claims 1-23, wherein the amount of the one or more proteolytic enzymes is in a proteolytic enzyme concentration to sample protein weight ratio of about 1:20 to about 1:40.
26. The method of any one of claims 1-25, wherein quenching the one or more proteolytic enzymes is performed using an acid.
27. The method of any one of claims 1-26, wherein the acid is formic acid (FA) or trifluoroacetic acid (TFA), or a mixture thereof.
28. The method of any one of claims 2-27, wherein the reduction technique is performed at a temperature of about 35° C. to about 70° C.
29. The method of any one of claims 2-28, wherein the reduction incubation time is at least about 20 minutes.
30. The method of any one of claims 2-29, wherein the reduction technique is performed at a temperature of about 60° C. for at least about 50 minutes.
31. The method of any one of claims 2-30, wherein the reduction technique is performed using a third thermal cycle, wherein the lid temperature during the third thermal cycle is at least about 2° C. higher than the temperature of the block temperature during the third thermal cycle.
32. The method of claim 31, wherein the third thermal cycle is performed in a thermal cycler comprising a lid temperature control element.
33. The method of any one of claims 1-22, wherein the reducing agent is dithiothreitol (DTT) or tris(2-carboxyethyl) phosphine (TCEP).
34. The method of claim 33, wherein DTT is added in an amount of about 10 mM to about 100 mM.
35. The method of any one of claims 2-34, wherein the alkylation technique is performed at a temperature of about 20° C. to about 37° C.
36. The method of any one of claims 2-35, wherein the alkylation incubation time is at least about 5 minutes.
37. The method of any one of claims 2-36, wherein the alkylation technique is performed at a temperature of about 20° C. to about 25° C. for at least about 30 minutes.
38 The method of any one of claims 2-37, wherein the alkylating agent is iodoacetamide (IAA).
39. The method of claim 38, wherein IAA is added in an amount of about 10 mM to about 200 mM.
40. The method of any one of claims 4-39, wherein quenching the alkylating agent comprises use of a neutralizing agent.
41. The method of claim 40, wherein the neutralizing agent is DTT.
42. The method of any one of claims 1-4 and 6-41, wherein the proteolytically digested sample is introduced to the LC-MS system without performing an offline desalting technique.
43. The method of any one of claims 1-4 and 6-42, wherein the period of diversion of the LC separation technique comprises about 1 to about 5 column volumes of the initial eluate that are diverted to waste.
44. The method of any one of claims 1-4, and 6-43, wherein the LC-MS technique is a high pressure LC-MS technique.
45. The method of any one of claims 1-4 and 6-43, wherein the LC-MS technique comprises multiple reaction monitoring.
46. The method of any one of claims 1-4 and 6-45, further comprising adding a standard to the proteolytically digested sample prior to the LC-MS technique.
47. The method of claim 46, wherein the standard is a stable isotope-internal standard (SI-IS) peptide mixture.
48. The method of any one of claims 1-47, wherein the biological sample is admixed with a buffer prior to the thermal denaturation technique.
49. The method of claim 48, wherein the buffer is ammonium bicarbonate.
50. The method of any one of claims 1-49, wherein the proteolytic glycopeptide comprises one or more sialic acid groups.
51. The method of any one of claims 33-50, wherein the proteolytically digested sample introduced to the liquid chromatography (LC) system comprises one or more of the DTT, the IAA, the iodide, and a disulfide bonded 6-membered ring, wherein the disulfide bonded 6-membered ring is a byproduct of DTT.

### Section 2—Reversed-Phase Proteolytic Digestion Clean-Up Techniques for Samples Containing a Glycosylated Polypeptide

1. A method for processing a proteolytically digested sample to produce a processed sample suitable for use in a liquid chromatography-mass spectrometry (LC-MS) analysis,
wherein the proteolytically digested sample comprises a plurality of proteolytic polypeptides comprising at least one proteolytic glycopeptide,
the method comprising:
performing one or more of the following:
(a) subjecting the proteolytically digested sample to a solid phase extraction column comprising a reversed-phase medium according to one or more conditions to associate at least a portion of the plurality of proteolytic polypeptides with the reversed-phase medium, the one or more conditions comprising:
(i) a polypeptide loading amount of about 50% or less of a binding capacity of the reversed-phase medium,
wherein the binding capacity of the reversed-phase medium is based on an insulin load having 10% or less breakthrough; or
(ii) a polypeptide loading concentration of about 0.6 μg/μL or less; or
(b) subjecting the reversed-phase medium comprising the associated proteolytic polypeptides to a wash buffer at a wash flow rate of about 0.1 column volumes/minute to about 2 column volumes/minute; and
subjecting the reversed-phase medium comprising the associated proteolytic polypeptides to an elution buffer to produce the processed sample.
2. The method of claim 1, wherein the one or more conditions comprises the polypeptide loading amount of about 50% or less of the binding capacity of the reversed-phase medium.
3 The method of claim 1 or 2, wherein the one or more conditions comprises the polypeptide loading concentration of about 0.6 μg/μL or less.
4. The method of any one of claim 1-3, wherein the performing comprises the subjecting the reversed-phase medium comprising the associated proteolytic polypeptides to the wash flow rate of about 0.1 column volumes/minute to about 2 column volumes/minute.
5. The method of any one of claims 1-4, wherein the column comprising the reversed-phase material has a medium volume of about 1 to about 10 μL.
6. The method of any one of claims 1-5, wherein the polypeptide loading amount is about 30 μg to about 200 μg.
7. The method of any one of claims 1-6, wherein the polypeptide loading amount is contained in a solution volume of at least about 100 μL.
8. The method of any one of claims 1-7, wherein the wash flow rate ranges from about 0.5 μL/minutes to about 10 μL/minute.
9. The method of any one of claims 1-8, wherein the reversed-phase medium comprises an alkyl-based moiety covalently bound to a solid phase.
10. The method of claim 9, wherein the alkyl-based moiety comprises an octadecyl carbon functional group (C18) covalently bound to the solid phase.
11. The method of claim 9, wherein the alkyl-based moiety comprises an octa carbon functional group (C8) covalently bound to the solid phase.
12. The method of claim 9, wherein the carbon alkyl-based moiety comprises a tetra carbon functional group (C4) covalently bound to the solid phase.
13. The method of any one of claims 9-12, wherein the solid phase comprises a silica material.
14. The method of any one of claims 1-8, wherein the reversed-phase medium comprises a hydrophobic polymer material.
15. The method of claim 14, wherein the hydrophobic polymer material comprises a phenyl moiety.
16. The method of claim 15, wherein the hydrophobic polymer material comprises a reaction product of divinylbenzene.
17. The method of claim 16, wherein the hydrophobic polymer material comprises poly(styrene-co-divinylbenzene).
18. The method of any one of claims 1-17, further comprising subjecting the reversed-phase medium comprising the associated proteolytic polypeptides to a wash buffer prior to subjecting the reversed-phase medium to the elution buffer.
19. The method of any one of claims 1-18, further comprising subjecting the processed sample comprising the elution buffer to a drying technique to produce a dried sample.
20. The method of any one of claims 1-19, further comprising reconstituting the dried sample to produce a reconstituted sample and inputting the reconstituted sample into a LC chromatography system of a LC-MS system to obtain mass spectrometry data.
21. The method of claim 20, further comprising identifying a polypeptide sequence of a glycopeptide from the mass spectrometry data.
22. The method of claim 21, further comprising identifying a glycan attachment site of the glycopeptide from the mass spectrometry data.
23. The method of claim 21 or 22, further comprising identifying a glycan structure of the glycopeptide from the mass spectrometry data.
24. The method of any one of claims 1-23, wherein the at least one glycopeptide comprises a glycan structure comprising one or more sialic acid moieties.
25. The method of any one of claims 1-24, wherein the proteolytically digested sample is obtained from a method for proteolytically digesting a biological sample comprising a glycoprotein.

### Section 3—Absorbent or Bibulous Members Having a Polypeptide Standard and Configured for Deposition of a Blood Sample and LC-MS Analysis of Glycopeptides Therefrom

1. A method for performing a liquid chromatography-mass spectrometry (LC-MS) analysis of a proteolytic glycopeptide derived from a blood sample deposited on a delimited zone of an absorbent or bibulous member
wherein the blood sample comprises a plurality of polypeptides comprising at least one glycoprotein,
the method comprising:
extracting at least a portion of the plurality of polypeptides and one or more extraction internal standards from the absorbent or bibulous member to obtain an extracted sample,
wherein the absorbent or bibulous member comprises the one or more extraction internal standards prior to deposition of the blood sample within the delimited zone, and
wherein at least one of the one or more extraction internal standards comprises a polypeptide standard;
subjecting the extracted sample or a derivative thereof to a proteolytic digestion technique to produce a proteolytically digested sample comprising the proteolytic glycopeptide;
introducing at least a portion of the proteolytically digested sample to a liquid chromatography (LC) system of a LC-MS system; and
performing the LC-MS analysis on at least the proteolytic glycopeptide and the one or more extraction internal standards.
2. The method of claim 1, wherein the performing the LC-MS analysis comprises measuring an abundance signal for the proteolytic glycopeptide and an abundance signal for the one or more extraction internal standards.
3. The method of claim 2, wherein the performing the LC-MS analysis further comprises calculating a concentration of the proteolytic glycopeptide based on a concentration of the one or more extraction internal standards prior to deposition on the absorbent or bibulous member, the abundance signal for the proteolytic glycopeptide, and the abundance signal for the one or more extraction internal standards.
4. The method of claim 1, wherein the absorbent or bibulous member is a dried blood spot card.
5. The method of any one of claims 1-4, further comprising determining an extraction efficiency based on the LC-MS analysis of at least one of the one or more extraction internal standards.
6. The method of any one of claims 1-5, further comprising determining a digestion efficiency based on the LC-MS analysis of at least one of the one or more extraction internal standards.
7. The method of any one of claims 1-6, further comprising assessing a sample migration pattern based on the LC-MS analysis of at least one of the one or more extraction internal standards.
8. The method of any one of claims 1-7, wherein the one or more extraction internal standards comprise a plurality of polypeptide standards, and wherein at least two of the plurality of polypeptide standards have different amino acid lengths.
9. The method of claim 8, wherein the amino acid lengths of the plurality of polypeptide standards of the one or more extraction internal standards range from 4 amino acid to 1500 amino acids.
10. The method of any one of claims 1-9, wherein the at least one polypeptide standard of the one or more extraction internal standards comprises at least one internal enzymatic cleavage site.
11. The method of any one of claims 1-10, wherein the one or more extraction internal standards comprise a plurality of polypeptide standards, wherein at least two of the plurality of polypeptide standards have different net hydrophobicities as based on a computation tool or partition coefficient analysis.
12. The method of claim 11, wherein the plurality of polypeptide standards having different net hydrophobicities comprises a hydrophobicity range of about −0.5 to about 1 according to the Grand average of hydropathicity index (GRAVY).
13. The method of any one of claims 1-12, wherein the at least one polypeptide standard of the one or more extraction internal standards comprises a C-terminal arginine or lysine.
14. The method of any one of claims 1-13, wherein the at least one polypeptide standard of the one or more extraction internal standards comprises an amino acid sequence that does not have homology to a peptide derived from the human proteome.
15. The method of any one of claims 1-14, wherein the at least one polypeptide standard of the one or more extraction internal standards is a synthetic polypeptide.
16. The method of any one of claims 1-15, wherein the at least one polypeptide standard of the one or more extraction internal standards comprises a stable heavy isotope label.
17. The method of any one of claims 1-16, wherein the at least one polypeptide standard of the one or more extraction internal standards comprises a sequence that is non-homologous to an endogenous polypeptide of an individual from which the blood sample originates.
18. The method of any one of claims 1-17, wherein the at least one polypeptide standard of the one or more extraction internal standards is an analog of an endogenous polypeptide of an individual from which the blood sample originates.
19. The method of claim 18, wherein the analog is a stable heavy isotope labeled analog.
20 The method of any one of claims 1-19, wherein the at least one polypeptide standard of the one or more extraction internal standards is a recombinantly expressed polypeptide.
21. The method of any one of claims 1-20, wherein the at least one polypeptide standard of the one or more extraction internal standards is a glycopolypeptide.
22. The method of any one of claims 1-21, wherein the at least one polypeptide standard of the one or more extraction internal standards is a polypeptide that does not substantially interact with hemoglobin.
23. The method of any one of claims 1-22, wherein the at least one polypeptide standard of the one or more extraction internal standards comprises at least a contiguous 4 amino acid sequence from SEQ ID NOS: 14-20.
24. The method of any one of claims 1-23, wherein the at least one polypeptide standard of the one or more extraction internal standards comprises a sequence is selected from the group consisting of SEQ ID NOS: 21-22.
25. The method of any one of claims 1-24, wherein the absorbent or bibulous member comprises a known amount of each of the one or more extraction internal standards.
26. The method of claim 26, wherein the known amount of each of the one or more extraction internal standards is about 0.05 ppm to about 5 ppm.
27. The method of any one of claims 1-27, wherein the one or more extraction internal standards are deposited and dried on the absorbent or bibulous member within an area having a surface area of about 1,000 mm² or less.
28. The method of claim 27, wherein the one or more extraction internal standard are deposited and dried on the absorbent or bibulous member within the delimited zone.
29. The method of any one of claims 1-28, wherein the extracting the at least the portion of the plurality of polypeptides and the one or more extraction internal standards from the absorbent or bibulous member comprises:
separating one or more portions of the absorbent or bibulous member from the absorbent or bibulous member,
wherein the one or more portions of the absorbent or bibulous member comprise at least a portion of the blood sample and the one or more extraction internal standards;
extracting at least the portion of the plurality of polypeptides and the one or more extraction internal standards from the one or more portions of the absorbent or bibulous member into an extraction solution; and
precipitating at least the portion of the plurality of polypeptides and the one or more extraction internal standards to obtain the extracted sample.
30. The method of claim 29, wherein the separating the one or more portions of the absorbent or bibulous member comprises punching the one or more portion of the absorbent or bibulous member using a punching device.
31. The method of claim 29 or 30, wherein each of the one or more portions separated from the absorbent or bibulous member have a surface area of about 2 mm² to about 100 mm².
32. The method of any one of claims 29-31, wherein the precipitating at least the portion of the plurality of polypeptides and the one or more extraction internal standards comprises subjecting the at least the portion of the plurality of polypeptides and the one or more extraction internal standards to ethanol.
33. The method of any one of claims 1-32, further comprising adding a solution to the extracted sample to resolubilize polypeptide content therein prior to subjecting the extracted sample or the derivative thereof to the proteolytic digestion technique.
34. The method of any one of claims 1-33, wherein the proteolytic digestion technique comprises a thermal denaturation technique.
35. The method of claim 34, wherein the proteolytic digestion technique further comprises a reduction technique and an alkylation technique.
36. The method of claim 34 or 35, wherein the proteolytic digestion technique comprises the use of one or more proteases.
37 The method of claim 36, wherein the protease is trypsin.
38. The method of any one of claims 1-32, further comprising adding one or more quantification internal standards after subjecting the extracted sample or the derivative thereof to a proteolytic digestion technique and prior to introducing at least the portion of the proteolytically digested sample to the liquid chromatography LC system of the LC-MS system.
39. The method of any one of claims 1-38, wherein the LC-MS analysis comprises a multiple-reaction-monitoring (MRM) technique targeting the proteolytic glycopeptide and the one or more extraction internal standards.
40. The method of claim 38 or 39, wherein the LC-MS analysis comprises a multiple-reaction-monitoring (MRM) technique targeting the one or more quantification internal standards.
41. The method of any one of claims 1-40, wherein the absorbent or bibulous member comprises a delimited zone having a surface area of about 1,000 mm² or less.
42. The method of any one of claims 1-41, wherein the absorbent or bibulous member comprises a filter paper material.
43. The method of claim 42, wherein the filter paper material comprises a cellulose-based paper.
44. The method of claim 42 or 43, wherein the filter paper material prevents or reduces sample hemolysis.
45. The method of any one of claims 1-44, wherein the absorbent or bibulous member comprises a lateral flow material configured to separate whole blood into a portion of plasma, wherein the whole blood is deposited at the delimited zone and then a liquid portion of the whole blood laterally flows from the delimited zone to a distal zone, wherein the distal zone contains the portion of the plasma.
46. An absorbent or bibulous member comprising one or more extraction internal standard deposited thereon on a delimited zone, wherein the one or more extraction internal standards comprises at least one polypeptide standard, and wherein the absorbent or bibulous member does not comprise a blood sample deposited thereon.
47. The absorbent or bibulous member of claim 46, wherein the absorbent or bibulous member is a blood spot card.

### Section 4—Method of Diagnosing Pelvic Tumors

1. A method of classifying a biological sample obtained from a subject with respect to a plurality of states associated with a pelvic cancer, the method comprising
receiving peptide structure data corresponding to a set of glycoproteins in the biological sample;
inputting quantification data identified from the peptide structure data for a set of peptide structures into a machine-learning model trained to identify a disease indicator based on the quantification data, wherein the set of peptide structures comprises at least one peptide structure identified from a plurality of peptide structures in Table 9;
identifying, by the machine-learning model, the disease indicator; and classifying the biological sample with respect to a plurality of states associated with pelvic cancer based upon the identified disease indicator.
2. A method of detecting the presence of one of a plurality of states associated with a pelvic cancer in a subject, the method comprising
receiving peptide structure data corresponding to a set of glycoproteins in a biological sample obtained from a subject, wherein the peptide structure data comprises at least one peptide structure from Table 9;
inputting quantification data identified from the peptide structure data for a set of peptide structures into a machine-learning model trained to identify a disease indicator based on the quantification data; and
detecting the presence of a corresponding state of the plurality of states associated with the pelvic cancer in response to a determination that the identified disease indicator falls within a selected range associated with the corresponding state.
3 The method of claim 1 or 2, wherein the plurality of states comprises at least one of a malignant tumor or a benign tumor.
4. The method of anyone of claims 1-3, wherein the machine-learning model comprises a logistic regression model.
5. The method of any one of claims 1-4, further comprising administering to the subject an effective amount of an agent to treat the pelvic tumor.
6. The method of any one of claims 1-5, wherein the pelvic tumor is ovarian cancer.
7. A method of treating a pelvic tumor in a subject comprising
receiving peptide structure data corresponding to a set of glycoproteins in a biological sample obtained from a subject, wherein the peptide structure data comprises at least one peptide structure from Table 9;
inputting quantification data for the at least one peptide structure into a machine-learning model trained to generate a risk score based on the quantification data;
outputting, by the machine-learning model, the quantification data using the machine learning model to generate a risk score,
administering an effective amount of an agent to treat the pelvic cancer based upon the risk score.
8. A method of determining a diagnosis for a pelvic tumor in a subject comprising
receiving peptide structure data corresponding to a set of glycoproteins in a biological sample;
inputting quantification data identified from the peptide structure data for a set of peptide structures into a machine-learning model trained to identify a disease indicator based on the quantification data, wherein the set of peptide structure data comprises at least one peptide structure identified from a plurality of peptide structures in Table 9;
identifying, by the machine-learning model, the disease indicator; and
determining a diagnosis for the pelvic tumor based upon the identified disease indicator.
9. The method of claim 8, wherein the diagnosis is the presence of a malignant tumor or a benign tumor.
10. A method of treating a pelvic tumor in a subject comprising
receiving peptide structure data corresponding to a set of glycoproteins in a biological sample;
inputting quantification data identified from the peptide structure data for a set of peptide structures into a machine-learning model trained to identify a disease indicator based on the quantification data, wherein the peptide structure data comprises at least one peptide structure identified from a plurality of peptide structures in Table 9;
identifying, by the machine-learning model, the disease indicator;
determining a risk score the identified disease indicator; and
administering an effective amount of an agent to treat the pelvic tumor based upon the risk score.
11. A method of treating a pelvic tumor in an individual comprising detecting the presence or amount of at least one peptide structure, wherein the at least one peptide structure comprises at least one peptide structure from Table 9, and administering an effective amount of an agent to treat the pelvic tumor based upon the presence or amount of the peptide structure.
12. A method of diagnosing an individual with a benign or malignant pelvic tumor comprising detecting a presence or amount of at least one peptide structure, wherein the at least one peptide structure comprises at least one peptide structure from Table 9, and diagnosing the individual with a benign or malignant pelvic tumor based upon the presence or amount of the at least one peptide structure.
13. A method of diagnosing an individual with a pelvic tumor comprising
detecting the presence or amount of at least one peptide structure from Table 9;
inputting a quantification of the detected at least one peptide structure into a machine-learning model trained to generate a class label,
determining if the class label is above or below a threshold for a classification;
identifying a diagnostic classification for the individual based on whether the class label is above or below a threshold for the classification; and
diagnosing the individual as having a benign or malignant pelvic tumor on the diagnostic classification.
14. The method of any one of claims 1-10, further comprising detecting the presence or amount of at least one peptide structure from Table 9.
15. The method of any one of claims 11-14, wherein the presence or amount of the at least one peptide structure is detected using mass spectrometry or ELISA.
16. The method of claim 15, wherein the presence or amount of the at least one peptide structure is detected using MRM mass spectrometry.
17. The method of any one of claims 11-16, wherein the amount of at least one peptide structure is none, or below a detection limit.
18. The method of any one of claims 1-17, wherein the at least one peptide structure comprises two or more peptide structures identified in Table 9, three or more peptides structures identified in Table 9, four or more peptide structure identified in Table 9, five or more peptide structures identified in Table 9, six or more peptide structures identified in Table 9, seven or more peptide structures identified in Table 9, or eight or more peptide structure identified in Table 9.
19. The method of any one of claims 1-18 wherein the at least one peptide structure comprises the sequence set forth in SEQ ID NOs: 35-51.
20. The method of any one of claims 1-19, wherein the at least one peptide structure comprises the sequence set forth in SEQ ID NOs: 35-42.
21. The method of any one of claims 1-19, wherein the at least one peptide structure comprises the sequence set forth in SEQ ID NOs: 43-51.
22. The method of any one of claims 1-19, wherein the at least one peptide structure comprises the sequence set forth in SEQ ID NOs: 35-40.
23. The method of any one of claims 1-22, wherein the biological sample is a blood sample, a serum sample, or tumor tissue.
24. The method of claim 23, wherein the biological sample is the blood sample, wherein the blood sample is deposited on a delimited zone of an absorbent or bibulous member comprising a plurality of polypeptides comprising at least one glycoprotein.
25. The method of claim 24 further comprising
extracting at least a portion of the plurality of polypeptides and one or more extraction internal standards from the absorbent or bibulous member to obtain an extracted sample,
wherein the absorbent or bibulous member comprises the one or more extraction internal standards prior to deposition of the blood sample within the delimited zone, and
wherein at least one of the one or more extraction internal standards comprises a polypeptide standard;
subjecting the extracted sample or a derivative thereof to a proteolytic digestion technique to produce a proteolytically digested sample comprising the proteolytic glycopeptide;
introducing at least a portion of the proteolytically digested sample to a liquid chromatography (LC) system of a LC-MS system; and
performing the LC-MS analysis on at least the proteolytic glycopeptide and the one or more extraction internal standards, wherein the at least one proteolytic glycopeptide comprises at least one peptide structure set forth in Table 9.
26. A method for performing a liquid chromatography-mass spectrometry (LC-MS) analysis of a proteolytic glycopeptide derived from a blood sample from an individual deposited on a delimited zone of a blood spot card, the method comprising
obtaining a blood spot card comprising a blood sample from the individual deposited thereon, wherein the blood spot card comprises one or more extraction internal standards deposited and dried prior to deposition of the blood sample on the blood spot card, and
wherein the blood spot card comprising the blood sample contains at least a portion of the blood sample and the one or more extraction internal standards in an overlapping area of the blood spot card;
extracting at least a portion of the plurality of polypeptides and the one or more extraction internal standards from the blood spot card to obtain an extracted sample;
subjecting the extracted sample or a derivative thereof to a proteolytic digestion technique to produce a proteolytically digested sample comprising the proteolytic glycopeptide;
introducing at least a portion of the proteolytically digested sample to a liquid chromatography (LC) system of a LC-MS system; and
performing an LC-MS analysis to quantify one or more biomarkers of ovarian cancer and the one or more extraction internal standards,
wherein the one or more biomarkers comprise a polypeptide comprising a sequence of any of SEQ ID NOs: 35-51, and
wherein at least one of the one or more biomarkers is a glycopeptide.
27. The method of claim 25 or 26, wherein the at least one polypeptide standard of the one or more extraction internal standards comprises at least a contiguous 4 amino acid sequence from SEQ ID NOs: 14-20.
28. The method of any one of claims 25-27, wherein the at least one polypeptide standard of the one or more extraction internal standards comprises a sequence is selected from the group consisting of SEQ ID NOs: 21-22.
29. The method of any one of claims 25-28, wherein the absorbent or bibulous member comprises a known amount of each of the one or more extraction internal standards.
30. The method of any one of claims 25-29, wherein extracting the at least the portion of the plurality of polypeptides and the one or more extraction internal standards from the absorbent or bibulous member comprises:
separating one or more portions of the absorbent or bibulous member from the absorbent or bibulous member,
wherein the one or more portions of the absorbent or bibulous member comprise at least a portion of the blood sample and the one or more extraction internal standards;
extracting at least the portion of the plurality of polypeptides and the one or more extraction internal standards from the one or more portions of the absorbent or bibulous member into an extraction solution; and
precipitating at least the portion of the plurality of polypeptides and the one or more extraction internal standards to obtain the extracted sample.
31. The method of claim 30, wherein the precipitating at least the portion of the plurality of polypeptides and the one or more extraction internal standards comprises subjecting at least the portion of the plurality of polypeptides and the one or more extraction internal standards to ethanol.
32. The method of any one of claims 25-31, further comprising adding a solution to the extracted sample to resolubilize polypeptide content therein prior to subjecting the extracted sample or the derivative thereof to the proteolytic digestion technique.
33. The method of any one of claims 25-32, wherein the proteolytic digestion technique comprises a thermal denaturation technique.
34. The method of claim 33, wherein the proteolytic digestion technique further comprises a reduction technique and an alkylation technique.
35. The method of claim 33 or 34, wherein the proteolytic digestion technique comprises the use of one or more proteases.
36. The method of claim 35, wherein the protease is trypsin.
37. The method of any one of claims 1-36, wherein the absorbent or bibulous member comprises a filter paper material.
38. The method of claim 37, wherein the filter paper material comprises a cellulose-based paper.
39. The method of claim 37 or 38, wherein the filter paper material prevents or reduces sample hemolysis.
40. The method of any one of claims 1-39, wherein the absorbent or bibulous member comprises a lateral flow material configured to separate whole blood into a portion of plasma, wherein the whole blood is deposited at the delimited zone and then a liquid portion of the whole blood laterally flows from the delimited zone to a distal zone, wherein the distal zone contains the portion of the plasma.
41. A method of training a model to diagnose a subject with one of a plurality of states associated with a pelvic tumor, the method comprising
receiving quantification data for a panel of peptide structures for a plurality of subjects diagnosed with the plurality of states associated with a pelvic tumor
wherein the panel of peptide structures comprises at least one peptide structure set forth in Table 9; and
training a machine-learning model to determine a state of the plurality of states a biological sample from the subject based on the quantification data.
42. The method of claims 1-10 and 41, wherein the quantification data comprises at least one of an abundance, a relative abundance, a normalized abundance, a relative quantity, an adjusted quantity, a normalized quantity, a relative concentration, an adjusted concentration, or a normalized concentration.
43. The method of claim 41 or claim 42, wherein the machine-learning model is trained using random forest or logical progression training methods.
44. The method of any one of claims 41-43, wherein training the machine-learning model to determine the state of the plurality of states comprises training the machine-learning model to generate a class label for the state of the plurality of states.
45. The method of any one of claims 41-44 wherein the machine-learning model comprises a logistic regression model.
46. The method of any one of claims 1-26 and 41-45, wherein at least one of the peptide structures comprises a glycopeptide.
47. A composition comprising one or more peptide structures from Table 9.
48. A composition comprising one or more peptides comprising the sequence set forth in SEQ ID NOs: 35-51.

### Section 5—HILIC Enrichment Sample Preparation for Quantitative Mass Spectrometry

1. A method for processing a proteolytic digest sample for use in a liquid chromatography-mass spectrometry (LC-MS) analysis,
wherein the proteolytic digest sample comprises a plurality of proteolytically digested peptides comprising at least one proteolytically digested glycopeptide,
the method comprising:
(A) loading a hydrophilic interaction liquid chromatography (HILIC) load derived from the proteolytic digest sample to a solid phase extraction column comprising a HILIC medium according to one or more conditions to associate the at least one proteolytically digested glycopeptide with the HILIC medium, the one or more conditions comprising:
(1) the loading of the HILIC load to the solid phase extraction column is initiated when the HILIC medium is in a dry state;
(2) the HILIC load loaded to the solid phase extraction column has an amount of the plurality of proteolytically digested peptides characterized by one or both of:
(a) a ratio of a weight of the plurality of proteolytically digested peptides over a weight of the HILIC medium in the dry state of at least about 0.06; and/or
(b) a ratio of the weight of the plurality of proteolytically digested peptides relative to a bed volume of the HILIC medium in the dry state of at least about 40 μg/μl; or
(3) the HILIC load loaded to the solid phase extraction column has a concentration of an organic solvent of at least about 70% (v/v); and
(B) subjecting the HILIC medium to an elution liquid to obtain a HILIC eluate comprising the at least one proteolytically digested glycopeptide.
2. The method of claim 1, wherein the one or more loading conditions comprise the loading of the HILIC load to the solid phase extraction column being initiated when the HILIC medium is in the dry state.
3. The method of claim 1 or 2, wherein the HILIC load is characterized by having the ratio of the weight of the plurality of proteolytically digested peptides over the weight of the HILIC medium in the dry state of at least about 0.06.
4. The method of any one of claims 1-3, wherein the HILIC load is characterized by having the ratio of the weight of the plurality of proteolytically digested peptides relative to the bed volume of the HILIC medium in the dry state of at least about 40 μg/μl.
5. The method of any one of claims 1-4, wherein the weight of the HILIC medium in the dry state is about 3 mg or the bed volume of the HILIC medium in the dry state is about 5 μL.
6. The method of claim 5, wherein the HILIC load is characterized by having the ratio of the weight of the plurality of proteolytically digested peptides over the weight of the HILIC medium in the dry state of about 0.1.
7. The method of claim 5 or 6, wherein the HILIC load is characterized by having the ratio of the weight of the plurality of proteolytically digested peptides relative to the bed volume of the HILIC medium in the dry state of about 60 μg/μl.
8. The method of claim 5, wherein the HILIC load is characterized by having the ratio of the weight of the plurality of proteolytically digested peptides over the weight of the HILIC medium in the dry state of about 0.2.
9. The method of claim 5 or 8, wherein the HILIC load is characterized by having the ratio of the weight of the plurality of proteolytically digested peptides relative to the bed volume of the HILIC medium in the dry state of about 120 μg/μl.
10. The method of any one of claims 1-9, wherein the one or more conditions comprise the HILIC load loaded to the solid phase extraction column having the concentration of the organic solvent of at least about 70% (v/v).
11. The method of any one of claims 1-10, wherein the HILIC medium comprises less than about 5% (v/v) of a liquid at the initiation of the loading of the HILIC load to the solid phase extraction column.
12. The method of claim 11, wherein, at the initiation of the loading of the HILIC load to the HILIC medium of the solid phase extraction column, the HILIC medium is not equilibrated with an equilibration liquid.
13. The method of any one of claims 1-12, wherein the HILIC load comprises an amount of the plurality of proteolytically digested peptides of at least about 200 μg.
14. The method of any one of claims 1-13, wherein the concentration of the organic solvent in the HILIC load is at least about 80% (v/v).
15. The method of any one of claims 1-14, wherein the organic solvent comprises an aprotic solvent miscible in water.
16. The method of any one of claims 1-15, wherein the organic solvent is selected from the group consisting of acetonitrile, ethanol, methanol, tetrahydrofuran, and dioxane, or a combination thereof.
17. The method of any one of claims 1-16, further comprising obtaining the HILIC load.
18. The method of claim 17, wherein obtaining the HILIC load comprises reducing a liquid content from the proteolytic digest sample without substantial loss of the plurality of proteolytically digested peptides in the proteolytic digest sample.
19. The method of claim 18, wherein the reducing the liquid content from the proteolytic digested sample comprises performing a peptide concentrating technique with the proteolytically digested sample to obtain a precursor of the HILIC load such that (a) the precursor can be reconstituted with a reconstitution liquid comprising the organic solvent to obtain the HILIC load having a volume of 220 μL or less and a concentration of the organic solvent of at least about 70% (v/v); and (b) the resulting HILIC load comprises an amount of the plurality of proteolytically digested peptides of at least about 200 μg.
20. The method of claim 17, further comprising:
reducing a liquid content from the proteolytic digest sample to form a dried proteolytic digest sample; and
reconstituting the dried proteolytic digest sample with a reconstitution liquid comprising the organic solvent to produce the HILIC load such that (a) the HILIC load has a volume of 220 μL or less and a concentration of the organic solvent of at least about 70% (v/v); and (b) the HILIC load has an amount of the plurality of proteolytic peptides of at least about 200 μg.
21. The method of claim 20, wherein the reconstituting the dried proteolytic digest sample comprises:
mixing the dried proteolytic digest sample with an amount of water to form a water mixture: sonicating the water mixture with a sonicator;
mixing the water mixture with an amount of trifluoracetic acid (TFA) and acetonitrile (ACN), wherein the amount of TFA and ACN are such that the final concentration of TFA is 1% (v/v) and the final concentration of ACN is 80% (v/v); and
sonicating the water mixture having the amount of TFA and ACN with a sonicator to produce the HILIC load.
22. The method of claim 21, wherein the sonicating the water mixture with the sonicator comprises a water-based dissolution cycle,
wherein the water-based dissolution cycle is repeated about 2 times to about 5 times,
and wherein for each of the water-based dissolution cycles, the sonicating the water mixture is performed for about 5 minutes and a water reservoir of the sonicator is configured with ice to cool the water reservoir.
23. The method of claim 21 or 22, wherein the sonicating the water mixture having the amount of TFA and ACN with the sonicator comprises an organic-based dissolution cycle, wherein the organic-based dissolution cycle is repeated about 2 times to about 3 times, and wherein for each of the organic-based dissolution cycles, the sonicating is performed for about 4 minutes and a water reservoir of the sonicator is configured with ice to cool the water reservoir.
24 The method of any one of claims 18-23, wherein the reducing the liquid content from the proteolytic digest sample comprises removing all or substantially all of the liquid content therefrom.
25 The method of any one of claims 18-23, wherein the peptide concentrating technique comprises a vacuum evaporation technique or a lyophilization technique.
26. The method of any one of claims 1-25, wherein the volume of the HILIC load is 220 μL or less.
27. The method of any one of claims 1-26, wherein the HILIC medium comprises a solid phase or a solid phase comprising a polar functional moiety.
28. The method of claim 27, wherein the solid phase comprises a silica material.
29 The method of claim 27 or 28, wherein the polar functional moiety comprises one or more of an amino group, a cyano group, a carbamoyl group, an aminoalkyl group, alkylamide group, or a combination thereof.
30. The method of any one of claims 1-29, further comprising performing a washing step after loading the HILIC load to the solid phase extraction column and prior to the subjecting the HILIC medium to the elution liquid, wherein the washing step comprises subjecting the HILIC medium to a wash liquid.
31. The method of any one of claims 1-30, further comprising collecting the HILIC eluate, or a fraction thereof, from the solid phase extraction column, wherein the HILIC eluate comprises the at least one proteolytically digested glycopeptide.
32. The method of claim 31, wherein after the collecting the HILIC eluate from the solid phase extraction column, the method further comprises reducing a liquid content of the collected HILIC eluate.
33. The method of any one of claims 1-32, further comprising subjecting the HILIC eluate to a peptide concentrating technique to produce a dried HILIC eluate.
34. The method of claim 33, further comprising reconstituting the dried HILIC eluate to form a sample suitable for introduction to the LC-MS system.
35. The method of claim 34, further comprising injecting the sample suitable for introduction to the LC-MS system into the LC-MS system.
36. The method of any one of claims 1-35, further comprising performing a mass spectrometry technique to obtain mass spectrometry data.
37. The method of claim 36, further comprising identifying a peptide sequence of a glycopeptide from the mass spectrometry data.
38. The method of claim 37, further comprising identifying a glycan attachment site of the glycopeptide from the mass spectrometry data.
39. The method of claim 37 or 38, further comprising identifying a glycan structure of the glycopeptide from the mass spectrometry data.
40. The method of any one of claims 1-39, wherein the at least one glycopeptide comprises a glycan structure comprising one or more sialic acid moieties.
41. The method of any one of claims 1-40, wherein the proteolytic digest sample is obtained from a method for proteolytically digesting a biological sample comprising a glycoprotein.
42. The method of any one of claims 1-41, wherein a glycopeptide concentration for a glycopeptide derived from the proteolytic digest sample is enriched by a factor of 30 or greater with respect to a peptide concentration, wherein the peptide concentration represents an amount of a peptide that is associated with the same protein as the glycopeptide.
43. The method of any one of claims 1-42, further comprising:
measuring a first plurality of peak area values for a first panel of glycopeptides;
measuring a second plurality of peak area values for a second panel of unglycosylated peptides wherein each of the unglycosylated peptides of the second panel corresponds to each of the glycopeptides of the first panel by being attached to a same protein molecule before a proteolytic digestion;
calculating a plurality of ratios by dividing each of the first plurality of peak area values with each of the second plurality of peak area values, respectively; and
determining a median ratio from the plurality of ratios, wherein the median ratio is greater than 30.

### Section 6—Fibrinogen-Depletion and Use Thereof in Glycoproteomic Analysis

1. A method of processing a blood-derived sample obtained from an individual for a glycoproteomic mass spectrometry (MS) technique, the method comprising:
(a) admixing the blood-derived sample with one or more defibrination factors to promote formation of a fibrin clot, the one or more defibrination factors comprises one or more members selected from the group consisting of:
a clotting co-factor;
a clotting enzyme; and
a clotting activator and/or an exogenous surface aggregation agent;
(b) separating the formed fibrin clot from the admixed blood-derived sample to obtain a fibrinogen-depleted sample; and
(c) subjecting the fibrinogen-depleted sample to one or more MS preparation techniques to produce a test sample for the glycoproteomic mass spectrometry technique.
2. The method of claim 1, wherein the one or more defibrination factors comprises the clotting co-factor.
3. The method of claim 2, wherein the clotting co-factor comprises a divalent cation.
4. The method of claim 3, wherein the clotting co-factor comprises the divalent cation, and wherein the divalent cation is Ca²⁺, Mg²⁺, Zn²⁺, or Cu²⁺, or any combination thereof.
5. The method of claim 3 or 4, wherein the divalent cation is Ca²⁺.
6. The method of any one of claims 2-5, wherein the clotting co-factor is calcium chloride, calcium acetate, calcium carbonate, calcium citrate, or calcium gluconate, or any combination thereof.
7. The method of any one of claims 2-6, wherein, following admixing with the blood-derived sample, the clotting co-factor has a concentration of about 5 mM to about 25 mM.
8. The method of any one of claims 1-7, wherein the one or more defibrination factors comprises the clotting enzyme.
9. The method of claim 8, wherein the clotting enzyme is thrombin.
10. The method of claim 8 or 9, wherein, following admixing with the blood-derived sample, the clotting enzyme has a concentration of about 1 unit/mL to 10 units/mL.
11. The method of any one of claim 1-10, wherein the one or more defibrination factors comprises the clotting activator and/or the exogenous surface aggregation agent.
12. The method of claim 11, wherein the clotting activator and/or the exogenous surface aggregation agent is an exogenous surface aggregation agent.
13. The method of claim 12, wherein the exogenous surface aggregation agent comprises Kaolin.
14. The method of claim 11, wherein the clotting activator and/or the exogenous surface aggregation agent is a clotting activator and exogenous surface aggregation agent.
15. The method of claim 14, wherein the clotting activator and exogenous surface aggregation agent comprises a material having pores with an average size of about 2 nm to about 60 nm.
16. The method of claim 14 or 15, wherein the clotting activator and exogenous surface aggregation agent comprises a silica particle.
17. The method of claim 16, wherein the silica particle has a pore size ranging from about 2 to about 60 nm.
18. The method of any one of claims 11-17, wherein the clotting activator and/or the exogenous surface aggregation agent is admixed with the blood-derived sample at an amount of about 50 μg to about 500 μg per 40 μL of the blood-derived sample.
19. The method of any one of claims 1-18, wherein the one or more defibrination factors comprise the clotting co-factor and the clotting enzyme.
20. The method of any one of claims 1-18, wherein the one or more defibrination factors comprise the clotting co-factor and the clotting activator and/or the exogenous surface aggregation agent.
21. The method of any one of claims 1-18, wherein the one or more defibrination factors comprise the clotting enzyme and the clotting activator and/or the exogenous surface aggregation agent.
22. The method of any one of claims 1-18, wherein the one or more defibrination factors comprise the clotting co-factor, the clotting enzyme, and the clotting activator and/or the exogenous surface aggregation agent.
23. The method of any one of claims 1-22, wherein more than one defibrination factor is admixed with the blood-derived sample sequentially.
24. The method of any one of claims 1-22, wherein more than one defibrination factor is admixed with the blood-derived sample simultaneously.
25. The method of any one of claims 1-24, wherein at least one of the one or more defibrination factors is added to a vessel containing the blood-derived sample.
26. The method of any one of claims 1-25, wherein the blood-derived sample is added to a vessel containing at least one of the one or more defibrination factors.
27. The method of any one of claims 1-26, wherein the method further comprises an incubation period following the admixing of the blood-derived sample with one or more defibrination factors.
28. The method of claim 27, wherein the incubation period is about 1 minute to about 30 minutes.
29 The method of any one of claims 1-28, wherein the separating the formed fibrin clot to obtain the fibrinogen-depleted sample comprises subjecting the admixed blood-derived sample with the one or more defibrination factors to a centrifugation technique and/or a filtration technique.
30. The method of any one of claims 1-29, wherein the separating the formed fibrin clot to obtain the fibrinogen-depleted sample comprises subjecting the admixed blood-derived sample with the one or more defibrination factors to a supernatant collection technique.
31. The method of any one of claims 1-30, wherein the fibrinogen-depleted sample is depleted of at least about 80% of the fibrinogen as compared to the blood-derived sample.
32. The method of any one of claims 1-31, wherein the fibrinogen-depleted sample is depleted of at least about 99% of the fibrinogen as compared to the blood-derived sample.
33. The method of any one of claims 1-32, wherein the blood-derived sample is a plasma sample.
34. The method of claim 33, wherein the plasma sample has been treated with an anticoagulant.
35. The method of claim 33 or 34, wherein the plasma sample has been treated with any one or more of the following: a citrate, an ACD (anticoagulant citrate dextrose), Streck, EDTA (ethylenediaminetetraacetic acid), Heparin or Li-Heparin, oxalate fluoride, or a citrate phosphate dextrose adenine (CPDA).
36. The method of any one of claims 1-35, wherein the blood-derived sample is a serum sample.
37. The method of any one of claims 1-36, wherein the one or more MS preparation techniques comprises subjecting the fibrinogen-depleted sample, or a derivative thereof, to a thermal denaturation technique.
38. The method of any one of claims 1-37, wherein the one or more MS preparation techniques comprises subjecting the fibrinogen-depleted sample, or a derivative thereof, to a proteolytic digestion technique.
39. The method of claim 38, wherein the proteolytic digestion technique comprises the use of one or more proteases.
40. The method of claim 39, wherein proteolytic digestion technique comprises the use of trypsin.
41. The method of claim 39 or 40, wherein the one or more proteases are present at a weight ratio of about 1:30 or less, relative to polypeptide content of the fibrinogen-depleted sample, or a derivative thereof.
42. The method of any one of claims 1-41, wherein the one or more MS preparation techniques comprises subjecting the fibrinogen-depleted sample, or a derivative thereof, to a desalting technique.
43. The method of any one of claims 1-42, further comprising performing the glycoproteomic mass spectrometry technique.
44. The method of any one of claims 1-43, wherein the glycoproteomic mass spectrometry technique comprises a liquid chromatography-mass spectrometry (MS) (LC-MS) technique.
45. The method of claim 44, wherein the LC-MS technique comprises a period of diversion of an initial eluate comprising a salt.
46. The method of any one of claims 1-45, wherein the glycoproteomic mass spectrometry technique comprises a multiple-reaction-monitoring (MRM) technique targeting a glycopeptide.
47. A method of preparing a plasma sample obtained from an individual for a glycoproteomic mass spectrometry technique, the method comprising:
(a) admixing the plasma sample with defibrination factors to promote formation of a fibrin clot, the defibrination factors comprising:
a clotting co-factor;
a clotting enzyme; and
a clotting activator and/or an exogenous surface aggregation agent;
(b) separating the formed fibrin clot from the admixed plasma sample to obtain a fibrinogen-depleted sample; and
(c) subjecting the fibrinogen-depleted sample to one or more MS preparation techniques to produce a test sample for the glycoproteomic mass spectrometry technique.
48. The method of claim 47, wherein, after following admixing with the blood-derived sample:
the clotting co-factor comprises Ca²⁺ at a concentration of about 5 mM to about 25 mM;
the clotting enzyme comprises thrombin at a concentration of about 1 unit/mL to 10 units/mL;
and
the clotting activator and/or the exogenous surface aggregation agent is in an amount of about 50 μg to about 500 μg per 40 μL of the blood-derived sample.
49. A defibrination composition comprising:
a clotting co-factor;
a clotting enzyme; and
a clotting activator and/or an exogenous surface aggregation agent.
50. A vessel comprising a defibrination composition of claim 49.

### Section 7—Methods and Systems for Analyzing Site-Specific Monomer Composition

1. A method for analyzing a set of peptide structures comprising a linking site, the method comprising:
   A) calculating a site occupancy score, for a given peptide structure at the linking site, as a function of an adjusted-raw abundance value for the given peptide structure and a sum of a set of adjusted-raw abundance values of the set of peptide structures; and
   B) calculating a monomer weight score as a sum of the site occupancy score and a multiplier, wherein the multiplier is the number of a specific monomer in the set of peptide structures at the linking site.
2. The method of claim 1, further comprising, prior to (A), receiving a set of raw abundance values of the set of peptide structures and normalizing the set of raw abundance values to a corresponding reference run to generate the set of adjusted-raw abundance values.
3. The method of claim 2, further comprising, prior to (B), calculating a peptide structure monomer weight score as a function of the site occupancy score and the number of a specific monomer for the given peptide structure.
4. The method of claim 3, wherein the monomer weight score is a function of the peptide structure monomer weight score and the site occupancy score.
5. The method of any one of claims 1-4, wherein the set of peptide structures is from a biological sample from a subject.
6. The method of claim 5, wherein the biological sample comprises serum or plasma samples.
7. The method of claim 6, wherein the reference run comprises serum or plasma samples.
8. The method of any one of claims 1-7, further comprising:
   correlating the monomer weight score with an indication or disease state to determine a hazard ratio for the indication or disease state, wherein the hazard ratio is used to update a risk profile of the subject for the indication or disease state.
9 The method of any one of claims 1-8, further comprising:
   generating a diagnosis output for the indication or disease state for the subject, using a predictive model, as a function of the monomer weight score, wherein the diagnosis output is one of a predictive probability or a risk score.
10. The method of claim 9, wherein the predictive model is a logistic regression model, wherein the predictive model generates at least one marker that is correlated with the indication or disease state.
11. The method of any one of claims 1-10, further comprising:
   calculating a site occupancy score, for a given peptide structure at the linking site, as the quotient of the adjusted-raw abundance value for the given peptide structure over the sum of the set of adjusted-raw abundance values.
12. The method of any one of claims 1-11, further comprising:
   calculating a peptide structure monomer weight score as a product of the site occupancy score and the number of specific monomers for the given peptide structure.
13. The method of claim 12, further comprising:
   calculating a monomer weight score for the subject as a sum of peptide structure monomer weight scores for each peptide structure at the linking site.
14. The method of any one of claims 1-13, further comprising:
   generating a diagnosis output, based on the monomer weight score, for an indication or disease state, wherein the diagnosis output classifies the biological sample as evidencing a state associated with a disease state progression and/or responsiveness to a specific therapy.
15. The method of any one of claims 1-14, wherein the set of raw abundance values is generated using multiple reaction monitoring mass spectrometry (MRM-MS).
16. The method of any one of claims 1-15, further comprising:
   generating a diagnosis output based on the monomer weight score for an indication or disease state, and
   generating a treatment output based on at least one of the diagnosis output.
17. The method of claim 16, wherein the treatment output comprises at least one of an identification of a treatment to treat the subject or a treatment plan.
18. The method of claim 17, wherein the treatment comprises at least one of radiation therapy, chemoradiotherapy, surgery, immunotherapy, hormone therapy, or a targeted drug therapy.
19. The method of claim 18, wherein the treatment comprises immunotherapy, wherein the immunotherapy is immune checkpoint blockade therapy.
20. The method of claim 19 wherein the immune checkpoint blockade therapy comprises ipilimumab, nivolumab, and/or pembrolizumab.
21. The method of any one of claims 1-20, further comprising generating a diagnosis output, wherein generating the diagnosis output comprises:
   generating a report identifying that the biological sample evidences the indication or disease state.
22. The method of any one of claims 1-21, wherein the specific monomer is selected from the group consisting of hexose, HexNac, fucose, and sialic acid.
23. The method of claim 22, wherein the specific monomer is selected from the group consisting of glucose, mannose, galactose, GlcNAc, GalNAc, fucose, NeuGc, and NeuAc.
24. The method of any one of claims 1-23, further comprising calculating a second monomer weight score as a sum of the site occupancy score and a second multiplier, wherein the second multiplier is the number of a second monomer in the set of peptide structures at the linking site, wherein the second monomer is different from the specific monomer.
25. The method of any one of claims 1-24, further comprising calculating a plurality of additional monomer weight scores as functions of the site occupancy score and a plurality of additional multipliers, wherein the plurality of additional multipliers are the number of a plurality of additional monomers in the set of peptide structures at the linking site.
26. A method of classifying a biological sample with respect to risk of melanoma progression and/or responsiveness to immune checkpoint inhibitor therapy, the method comprising:
   A) analyzing one or more monomer weight scores of a set of peptide structures from a biological sample from the subject using a machine learning model to generate a disease indicator; and
   B) generating a diagnosis output based on the disease indicator that classifies the biological sample as evidencing a state associated with melanoma progression and/or responsiveness to immune checkpoint inhibitory therapy.
27. The method of claim 26, further comprising:
   receiving a set of raw abundance values of the set of peptide structures and normalizing the set of raw abundance values to a corresponding reference run to generate the set of adjusted-raw abundance values.
28. The method claim 27, further comprising:
   calculating a site occupancy score, for a given peptide structure at the linking site, as the function of the adjusted-raw abundance value for the given peptide structure and the sum of the set of adjusted-raw abundance values.
29. The method of claim 27 or 28, further comprising:
   calculating a site occupancy score, for a given peptide structure at the linking site, as the quotient of the adjusted-raw abundance value for the given peptide structure over the sum of the set of adjusted-raw abundance values.
30. The method of any one of claims 28-29, further comprising:
   calculating a peptide structure monomer weight score as a function of the site occupancy score and the number of specific monomers for the given peptide structure.
31. The method of any one of claims 28-30, further comprising:
   calculating a peptide structure monomer weight score as a product of the site occupancy score and the number of specific monomers for the given peptide structure.
32. The method of any one of claims 30-31, further comprising:
   calculating a monomer weight score of the one or more monomer weight scores as a sum of peptide structure monomer weight scores for each peptide structure at the linking site.
33. The method of any one of claims 26-32, wherein the set of peptide structures comprises post translationally modified (PTM) peptides and/or non-PTM peptides.
34. The method of any one of claims 26-33, wherein the monomer is selected from the group consisting of hexose, HexNac, fucose, and sialic acid.
35. The method of claim 34, wherein the monomer is selected from the group consisting of glucose, mannose, galactose, GlcNAc, GalNAc, fucose, NeuGc, and NeuAc.
36. The method of any one of claims 26-35, wherein the set of peptides structures comprises glycosylated peptides and non-glycosylated peptides.
37. The method of any one of claims 26-36, wherein the biological sample comprises serum or plasma samples.
38. The method of any one of claims 27-37, wherein the reference run comprises serum or plasma samples.
39. The method of any one of claims 26-38, further comprising:
   treating the biological sample to form a prepared sample comprising the set of peptide structures, the set of peptide structures comprising a set of post translationally modified (PTM) peptides and/or non-PTM peptides;
   detecting a set of product ions associated with each structure of the set of post translationally modified (PTM) peptides and/or non-PTM peptides, and
   generating the set of raw abundance values for the set of product ions.
40. The method of any one of claims 26-39, wherein the analyzing further comprises:
   correlating the monomer weight score with a melanoma disease state to determine a hazard ratio for the melanoma disease state, wherein the hazard ratio is used to update a risk profile of the subject for the melanoma disease state.
41. The method of any one of claims 26-40, further comprising:
   generating a diagnosis output based on the disease indicator that classifies the biological sample as evidencing a state associated with melanoma progression and/or responsiveness to immune checkpoint inhibitory therapy, wherein the diagnosis output is one of a predictive probability or a risk score.
42. The method of any one of claims 26-41, wherein the set of raw abundance values is generated using multiple reaction monitoring mass spectrometry (MRM-MS).
43. The method of any one of claims 26-42, further comprising:
   generating a treatment output based on at least one of the diagnosis output.
44. The method of claim 43, wherein the treatment output comprises at least one of an identification of a treatment to treat the subject or a treatment plan.
45. The method of claim 44, wherein the treatment comprises at least one of radiation therapy, chemoradiotherapy, surgery, hormone therapy, or a targeted drug therapy.
46. The method of any one of claims 26-45, wherein generating the diagnosis output comprises:
   generating a report identifying that the biological sample evidences the indication or disease state.
47. The method of any one of claims 26-46, wherein the one or more monomer weight scores correspond to at least one site monomer identified in Table 16.
48. The method of any one of claims 26-46, wherein the one or more monomer weight scores correspond to at least one site monomer identified in Table 17.
49. The method of any one of claims 26-46, wherein the one or more monomer weight scores correspond to at least one site monomer identified in Table 18.
50. The method of any one of claims 26-49, further comprising:
   training the at least one supervised machine learning model using training data,
   wherein the training data comprises a plurality of peptide structure profiles for a plurality of subjects and a plurality of subject diagnoses for the plurality of subjects.
51. The method of claim 50, wherein the plurality of subject diagnoses is selected from the group consisting of a positive diagnosis for any subject of the plurality of subjects determined to have a melanoma disease state, a negative diagnosis for any subject of the plurality of subjects determined not to have a melanoma disease state, a positive diagnosis for any subject of the plurality of subjects determined to be likely to benefit from immune checkpoint inhibitory therapy, and a negative diagnosis for any subject of the plurality of subjects determined to be unlikely to benefit from immune checkpoint inhibitory therapy.
52. The method of claim 51, wherein the plurality of subjects are separated into classes of positive and negative diagnoses using a concordance index as a cutoff between positive and negative diagnoses.
53. The method of any one of claims 50-52, further comprising:
   performing a differential expression analysis using the training data to compare a first portion of the plurality of subjects with the positive diagnosis for melanoma disease state or subjects unlikely to benefit from immune checkpoint inhibitory therapy, versus a second portion of the plurality of subjects having the negative diagnosis for melanoma disease state or subjects likely to benefit from immune checkpoint inhibitory therapy; and
   identifying a training group of peptide structures based on the differential expression analysis for use as prognostic markers for the melanoma disease state and/or responsiveness to immune checkpoint inhibitory therapy; and
   forming the training data based on the training group of peptide structures identified.
54. The method of any one of claims 26-53, wherein the at least one supervised machine learning model comprises a logistic regression model, and wherein the at least one supervised learning model compares the negative diagnosis versus the positive diagnosis, wherein the comparison can be at least one non-melanoma state vs at least one melanoma state, or the comparison can be at least one positive response to immune checkpoint inhibitory therapy vs at least one negative response to immune checkpoint inhibitory therapy.
55. A method of treating melanoma in a subject, the method comprising:
   A) analyzing one or more monomer weight scores corresponding to at least one site monomer identified in Table 16 using a machine learning model to generate a diagnosis output that classifies the biological sample as evidencing a state associated with melanoma progression, and
   B) administering a therapeutically effective amount of a treatment for melanoma.
56. The method of claim 55, further comprising:
   receiving a set of raw abundance values of the set of peptide structures and normalizing the set of raw abundance values to a corresponding reference run to generate the set of adjusted-raw abundance values.
57. The method of claim 56, further comprising:
   calculating a site occupancy score, for a given peptide structure at the linking site, as the function of the adjusted-raw abundance value for the given peptide structure and the sum of the set of adjusted-raw abundance values.
58 The method claim 56 or 57, further comprising:
   calculating a site occupancy score, for a given peptide structure at the linking site, as the quotient of the adjusted-raw abundance value for the given peptide structure over the sum of the set of adjusted-raw abundance values.
59. The method of any one of claims 57-58, further comprising:
   calculating a peptide structure monomer weight score as a function of the site occupancy score and the number of specific monomers for the given peptide structure.
60. The method of any one of claims 57-59, further comprising:
   calculating a peptide structure monomer weight score as a product of the site occupancy score and the number of specific monomers for the given peptide structure.
61. The method of claim 59 or 60, further comprising:
   calculating the a monomer weight score of the one or more monomer weight scores as a sum of peptide structure monomer weight scores for each peptide structure at the linking site.
62. The method of any one of claims 55-61, wherein the set of peptide structures comprises post translationally modified (PTM) peptides and/or non-PTM peptides.
63. The method of any one of claims 55-62, wherein the monomer is selected from the group consisting of hexose, HexNac, fucose, and sialic acid.
64. The method of claim 63, wherein the monomer is selected from the group consisting of glucose, mannose, galactose, GlcNAc, GalNAc, fucose, NeuGc, and NeuAc.
65. The method of any one of claims 55-64, wherein the set of peptides structures comprises glycosylated peptides and non-glycosylated peptides.
66. The method of any one of claims 55-65, wherein the biological sample comprises serum or plasma samples.
67. The method of any one of claims 56-66, wherein the reference run comprises serum or plasma samples.
68. The method of any one of claims 55-67, further comprising:
   treating the biological sample to form a prepared sample comprising the set of peptide structures, the set of peptide structures comprising a set of post translationally modified (PTM) peptides and/or non-PTM peptides;
   detecting a set of product ions associated with each structure of the set of post translationally modified (PTM) peptides and/or non-PTM peptides, and
   generating the set of raw abundance values for the set of product ions.
69. The method of any one of claims 55-68, wherein the analyzing further comprises:
   correlating the one or more monomer weight scores with a melanoma disease state to determine a hazard ratio for the melanoma disease state, wherein the hazard ratio is used to update a risk profile of the subject for the melanoma disease state.
70. The method of any one of claims 55-69, further comprising:
   generating a diagnosis output based on a disease indicator that classifies the biological sample as evidencing a state associated with melanoma progression, wherein the diagnosis output is one of a predictive probability or a risk score.
71. The method of any one of claims 55-70, wherein the set of raw abundance values is generated using multiple reaction monitoring mass spectrometry (MRM-MS).
72. The method of any one of claims 55-71, wherein the treatment comprises at least one of radiation therapy, chemoradiotherapy, immunotherapy, surgery, hormone therapy, or a targeted drug therapy.
73. The method of claim 72, wherein the treatment comprises immunotherapy, wherein the immunotherapy is immune checkpoint blockade therapy.
74. The method of claim 73 wherein the immune checkpoint blockade therapy comprises ipilimumab, nivolumab, and/or pembrolizumab.
75. The method of any one of claims 55-74, wherein generating the diagnosis output comprises:
   generating a report identifying that the biological sample evidences the indication or disease state.
76. The method of any one of claims 55-75, wherein the one or more monomer weight scores correspond to at least one site monomer identified in Table 17.
77. The method of any one of claims 55-75, wherein the one or more monomer weight scores correspond to at least one site monomer identified in Table 18.
78. The method of any one of claims 55-77, further comprising:
   training the at least one supervised machine learning model using training data,
   wherein the training data comprises a plurality of peptide structure profiles for a plurality of subjects and a plurality of subject diagnoses for the plurality of subjects.
79. The method of claim 78, wherein the plurality of subject diagnoses is selected from the group consisting of a positive diagnosis for any subject of the plurality of subjects determined to have a melanoma disease state, a negative diagnosis for any subject of the plurality of subjects determined not to have a melanoma disease state, a positive diagnosis for any subject of the plurality of subjects determined to be likely to benefit from immune checkpoint inhibitory therapy, and a negative diagnosis for any subject of the plurality of subjects determined to be unlikely to benefit from immune checkpoint inhibitory therapy.
80. The method of claim 79, wherein the plurality of subjects are separated into classes of positive and negative diagnoses using a concordance index as a cutoff between positive and negative diagnoses.
81. The method of any one of claims 78-80, further comprising:
   performing a differential expression analysis using the training data to compare a first portion of the plurality of subjects with the positive diagnosis for melanoma disease state or subjects unlikely to benefit from immune checkpoint inhibitory therapy, versus a second portion of the plurality of subjects having the negative diagnosis for melanoma disease state or subjects likely to benefit from immune checkpoint inhibitory therapy; and
   identifying a training group of peptide structures based on the differential expression analysis for use as prognostic markers for the melanoma disease state and/or responsiveness to immune checkpoint inhibitory therapy; and
   forming the training data based on the training group of peptide structures identified.
82. The method of any one of claims 55-81, wherein the at least one supervised machine learning model comprises a logistic regression model, and wherein the at least one supervised learning model compares the negative diagnosis versus the positive diagnosis, wherein the comparison can be at least one non-melanoma state vs at least one melanoma state, or the comparison can be at least one positive response to immune checkpoint inhibitory therapy vs at least one negative response to immune checkpoint inhibitory therapy.
83. A system comprising:
   one or more data processors; and
   a non-transitory computer readable storage medium containing instructions which, when executed on the one or more data processors, cause the one or more data processors to perform part or all of the method of any one of claims 1-82.
84. A computer-program product tangibly embodied in a non-transitory machine-readable storage medium, including instructions configured to cause one or more data processors to perform part or all of the method of any one of claims 1-82.
85. A method of monitoring a subject for a melanoma, the method comprising:
   receiving first monomer weight score data for a first biological sample obtained from a subject at a first timepoint;
   analyzing the first monomer weight score data using at least one supervised machine learning model to generate a first disease indicator based on at least one site monomer selected from a group of site monomers identified in Table 16, wherein the group of site monomers in Table 16 comprises a group of site monomers having monomer weight scores associated with melanoma;
   receiving second monomer weight score data of a second biological sample obtained from the subject at a second timepoint;
   analyzing the second monomer weight score data using the at least one supervised machine learning model to generate a second disease indicator based on the at least one site monomer selected from the group of site monomers identified in Table 16; and
   generating a diagnosis output based on the first disease indicator and the second disease indicator.
86. The method of claim 85, wherein generating the diagnosis output comprises: comparing the second disease indicator to the first disease indicator.
87. The method of claim 85 or 86, wherein the first disease indicator indicates that the first biological sample evidences a negative diagnosis for melanoma and the second biological sample evidences a positive diagnosis for melanoma.
88. The method of claim 85 or 86, wherein the first disease indicator indicates that the first biological sample evidences a melanoma that is not responsive to immunotherapy and the second biological sample evidences a melanoma that is responsive to immunotherapy.
89. The method of any one of claims 85-88, wherein the at least one supervised machine learning model comprises a logistic regression model, and wherein the at least one supervised learning model compares negative diagnoses versus positive diagnoses, wherein the comparison can be at least one healthy state versus melanoma generally, healthy state versus immunotherapy responsive melanoma, or immunotherapy nonresponsive melanoma versus immunotherapy responsive melanoma.
90. The method of any one of claims 85-89, wherein the at least one site monomer comprises at least one site monomer identified in Table 18.
91. The method of any one of claims 85-89, wherein the at least one site monomer comprises at all site monomers identified in Table 18.
92. A method of treating melanoma in a subject, the method comprising:
   determining a monomer weight score for at least one site monomer identified in Table 16 in a biological sample from the subject using a multiple reaction monitoring mass spectrometry (MRM-MS) system;
   analyzing the monomer weight score using at least one machine learning model to generate a disease indicator;
   generating a diagnosis output based on the disease indicator that classifies the biological sample as evidencing that the patient has melanoma; and
   administering to the subject a therapeutically effective amount of a melanoma therapy.
93. The method of claim 92, wherein the melanoma therapy comprises radiation therapy, chemotherapy, chemoradiotherapy, surgery, hormone therapy, immunotherapy, or a targeted drug therapy.
94. The method of claim 93, wherein the melanoma therapy comprises immunotherapy.
95. The method of claim 94, wherein the immunotherapy comprises immune checkpoint blockade therapy.
96. The method of claim 19 wherein the immune checkpoint blockade therapy comprises ipilimumab, nivolumab, and/or pembrolizumab.
97. The method of claim 92, wherein the melanoma therapy does not comprise immunotherapy.
98. The method of any one of claims 92-97, further comprising: preparing the biological sample to form a prepared sample comprising a set of peptide structures; and inputting the prepared sample into the MRM-MS system using a liquid chromatography system.
99. The method of any one of claims 92-98, wherein the at least one site monomer comprises at least one site monomer identified in Table 17.
100. The method of any one of claims 92-98, wherein the at least one site monomer comprises at least one site monomer identified in Table 18.
101. The method of any one of claims 92-98, wherein the at least one site monomer comprises at all site monomers identified in Table 18.
102. A method of treating melanoma in a subject, the method comprising:
   determining a monomer weight score for at least one site monomer identified in Table 16 in a biological sample from the subject using a multiple reaction monitoring mass spectrometry (MRM-MS) system;
   analyzing the monomer weight score using at least one machine learning model to generate a disease indicator;
   generating a diagnosis output based on the disease indicator that classifies the biological sample as evidencing that the melanoma is sensitive to immunotherapy; and
   administering to the subject a therapeutically effective amount of immunotherapy.
103. The method of claim 102, wherein the immunotherapy comprises immune checkpoint blockade therapy.
104. The method of claim 103 wherein the immune checkpoint blockade therapy comprises ipilimumab, nivolumab, and/or pembrolizumab.
105. The method of claim 102, 103, or 104, wherein the at least one site monomer comprises at least one site monomer identified in Table 17.
106. The method of claim 102, 103, or 104, wherein the at least one site monomer comprises at least one site monomer identified in Table 18.
107. The method of claim 102, 103, or 104, wherein the at least one site monomer comprises at all site monomers identified in Table 18.
108. A method of identifying a need for one or more medical tests for a subject suspected of being at risk for or having melanoma, the method comprising: subjecting the subject to the one or more medical tests in response to measuring that a biological sample obtained from the subject evidences the subject as having melanoma using part or all of the method of any one of claims 1-82.
109. The method of claim 108, wherein the one or more medical tests comprises colonoscopy, physical exam, CT scan, MRI scan, PET scan, or a combination thereof.
110. A method of designing a treatment for a subject having melanoma, the method comprising: designing a therapeutic regimen for treating the subject in response to measuring that a biological sample obtained from the subject evidences the subject as having melanoma using part or all of the method of any one of claims 1-82.
111. The method of claim 110, wherein the treatment comprises at least one of radiation therapy, chemotherapy, chemoradiotherapy, immunotherapy, surgery, hormone therapy, or a targeted drug therapy.
112. A method of treating a subject diagnosed with melanoma, the method comprising: administering to the subject immunotherapy to treat the subject based on measuring that a biological sample obtained from the subject evidences the melanoma as being sensitive to immunotherapy using part or all of the method of any one of claims 1-82.
113. The method of claim 112, wherein the immunotherapy comprises immune checkpoint blockade therapy.
114. A method of classifying a sample from an individual suspected of having, known to have, or at risk for melanoma, comprising the step of determining from the sample a monomer weight score for one or more of the site monomers in Table 16.
115. The method of claim 114, wherein the measuring identifies the individual as not having melanoma.
116. The method of claim 114, wherein the measuring identifies the individual as having melanoma.
117. The method of claim 116, further comprising administering to the individual an effective amount of at least one of radiation therapy, chemotherapy, chemoradiotherapy, immunotherapy, surgery, hormone therapy, or a targeted drug therapy.
118. The method of claim 114, wherein the measuring identifies the individual as having melanoma that is sensitive to immunotherapy.
119. The method of claim 114, wherein the measuring identifies the individual as having melanoma that is not sensitive to immunotherapy.
120. The method of any one of claims 114-119, wherein the sample comprises peripheral blood, plasma, or serum.
121. The method of any one of claims 114-120, wherein the individual is at risk for melanoma.
122. The method of any one of claims 114-121, wherein a monomer weight score is determined for one or more of the site monomers identified in Table 17.
123. The method of any one of claims 114-121, wherein a monomer weight score is determined for one or more of the site monomers identified in Table 18.
124. The method of claim 123, wherein a monomer weight score is determined for all site monomers identified in Table 18.
125. A method of predicting a risk for melanoma in a subject, the method comprising:
   determining a monomer weight score for at least one site monomer identified in Table 16 in a biological sample from the subject using a multiple reaction monitoring mass spectrometry (MRM-MS) system;
   analyzing the monomer weight score using at least one machine learning model to generate a disease indicator; and
   generating a diagnosis output based on the disease indicator that classifies the biological sample as evidencing that the patient has a risk for melanoma.
126. The method of claim 125, wherein the at least one site monomer comprises at least one site monomer identified in Table 17.
127. The method of claim 125, wherein the at least one site monomer comprises at least one site monomer identified in Table 18.
128. The method of claim 125, wherein the at least one site monomer comprises at all site monomers identified in Table 18.
129. A method of predicting immunotherapy sensitivity, the method comprising:
   determining a monomer weight score for at least one site monomer identified in Table 16 in a biological sample from the subject using a multiple reaction monitoring mass spectrometry (MRM-MS) system;
   analyzing the monomer weight score using at least one machine learning model to generate a disease indicator; and
   generating a diagnosis output based on the disease indicator that classifies the biological sample as evidencing that the patient has a risk for melanoma.
130. The method of claim 129, wherein the at least one site monomer comprises at least one site monomer identified in Table 17.
131. The method of claim 129, wherein the at least one site monomer comprises at least one site monomer identified in Table 18.
132. The method of claim 129, wherein the at least one site monomer comprises at all site monomers identified in Table 18.
133. A method of classifying a biological sample with respect to a responsiveness to immune checkpoint inhibitor therapy, the method comprising:
   A) analyzing one or more monomer weight scores of a set of peptide structures from a biological sample from the subject using a machine learning model to generate a disease indicator; and
   B) generating a diagnosis output based on the disease indicator that classifies the biological sample as evidencing the responsiveness to immune checkpoint inhibitory therapy, wherein the one or more monomer weight scores correspond to at least one site monomer identified in Table 29.
134. A method of classifying a biological sample with respect to a responsiveness to immune checkpoint inhibitor therapy, the method comprising:
   A) analyzing one or more monomer weight scores of a set of peptide structures from a biological sample from the subject using a machine learning model to generate a disease indicator;
   B) generating a diagnosis output based on the disease indicator that classifies the biological sample as evidencing the responsiveness to immune checkpoint inhibitory therapy, wherein the one or more monomer weight scores correspond to at least one site monomer identified in Table 29; and
   C) administering to the subject a therapeutically effective amount of immunotherapy.
135. A method for managing a treatment for a subject diagnosed with a melanoma, the method comprising:
receiving peptide structure data corresponding to a set of glycoproteins in a biological sample obtained from the subject;
computing a treatment score using quantification data identified from the peptide structure data for a set of peptide structures, wherein the set of peptide structures includes at least one peptide structure identified from a plurality of peptide structures listed in Table 23A;
generating a treatment output that indicates a predicted response to the treatment for the subject using the treatment score.
136. The method of claim 135, wherein the at least one peptide structure of Table 23A includes a glycan symbol structure or a glycan composition in accordance with Table 23C.

### Section 8—Methods and Systems for Analyzing Site-Specific Monomer Composition

1. A method for predicting retention times of peptides, by a computing system comprising one or more processors:
accessing a feature set corresponding to a peptide, wherein the feature set represents peptide sequence data of the peptide and corresponding physicochemical features;
sending the feature set as an input into a neural network, the neural network comprising: (1) a plurality of 1DCNN layers, (2) one or more BiLSTM layers, and (3) a multi-head attention layer;
and
obtaining, as an output from the neural network, a predicted retention time for the peptide corresponding to an estimated retention time for the peptide in a liquid chromatography mass spectrometry (LC-MS) run.
2. The method of claim 1, wherein the neural network further comprises a flatten and dense layer as a final output layer.
3. The method of claim 1, wherein the feature set for a peptide is generated by:
encoding a peptide sequence of the peptide to generate a matrix representation of the peptide; compressing the matrix representation to a vector representation; and
concatenating, to the vector representation, one or more corresponding physiochemical features that are determined to be associated with the peptide or peptide sequence.
4. The method of claim 3, wherein generating the feature set further comprises normalizing the concatenated vector representation between 0 and 1.
5. The method of claim 3, wherein the peptide sequence data is encoded using one-hot encoding.
6. The method of claim 5, wherein the matrix representation comprises:
20 columns corresponding to 20 unique amino acids, and
n rows, wherein each row corresponds to a position in a sequence of the corresponding peptide,
and wherein n corresponds to a length of the corresponding peptide.
7. The method of claim 3, wherein the peptide sequence data is encoded using BLOSUM 62.
8. The method of claim 7, wherein the encoding generates a matrix comprising:
20 columns corresponding to 20 unique amino acids;
3 columns corresponding to 3 special amino acid characters; and
1 column corresponding to a translation stop.
9. A method of training a neural network for predicting retention times of peptides, by a computing system comprising one or more processors:
accessing a plurality of feature sets corresponding to a plurality of peptides, wherein the feature set represents peptide sequence data of the peptide and corresponding physicochemical features; creating a training set comprising a subset of feature sets from the plurality of feature sets; and training a neural network using the training set, the neural network comprising: (1) a plurality of 1DCNN layers, (2) one or more BiLSTM layers, and (3) a multi-head attention layer.
10. The method of claim 9, further comprising:
creating a validation set comprising a subset of feature sets from the plurality of feature sets;
sending the validation set through the neural network; and
evaluating the outputs.
11. The method of claim 10, wherein the training set comprises 80% of the plurality of feature sets and the validation set comprises 30% of the plurality of feature sets.
12. A system for predicting retention times of peptides, the system comprising:
one or more processors; and
a non-transitory memory coupled to the processors comprising instructions executable by the processors, the processors operable when executing the instructions to:
access a feature set corresponding to a peptide, wherein the feature set represents peptide sequence data of the peptide and corresponding physicochemical features;
send the feature set as an input into a neural network, the neural network comprising: (1) a plurality of 1DCNN layers, (2) one or more BiLSTM layers, and (3) a multi-head attention layer.
obtain, as an output from the neural network, a predicted retention time for the peptide corresponding to an estimated retention time for the peptide in a liquid chromatography mass spectrometry (LC-MS) run.
13. A system for training a neural network for predicting retention times of peptides, the system comprising:
one or more processors; and
a non-transitory memory coupled to the processors comprising instructions executable by the processors, the processors operable when executing the instructions to:
access a plurality of feature sets corresponding to a plurality of peptides, wherein the feature set represents peptide sequence data of the peptide and corresponding physicochemical features;
create a training set comprising a subset of feature sets from the plurality of feature sets; and
train a neural network using the training set, the neural network comprising: (1) a plurality of 1DCNN layers, (2) one or more BiLSTM layers, and (3) a multi-head attention layer.
14. A computer-readable medium comprising instructions thereon, which when executed by a processor causes the processor to perform the method of any one of claims 1 to 11.

What is claimed is:

1. A method for performing a liquid chromatography-mass spectrometry analysis of a proteolytic glycopeptide derived from a biological sample comprising a glycoprotein, the method comprising:
subjecting the biological sample to a thermal denaturation technique to produce a denatured sample followed by a proteolytic digestion technique to produce a proteolytically digested sample comprising the proteolytic glycopeptide,
wherein the thermal denaturation technique subjects the biological sample to a thermal cycle comprising a thermal treatment of about 60° C. to about 100° C. with a hold time of at least about 1 minute,
wherein a lid temperature during the thermal cycle is at least about 2° C. higher than a temperature of a block temperature during the thermal cycle,
wherein the proteolytic digestion technique comprises adding an amount of one or more proteolytic enzymes and incubating for a digestion incubation time, and
wherein the proteolytic digestion technique comprises quenching the one or more proteolytic enzymes following the digestion incubation time;
introducing the proteolytically digested sample to a liquid chromatography (LC) system of a LC-MS system; and
performing a LC-MS technique to introduce the proteolytic glycopeptide to a mass spectrometer (MS) system,
wherein the LC-MS technique comprises a period of diversion of an initial eluate comprising a salt, and
wherein the LC system comprises a reversed-phase chromatography column.

2. The method of claim 1, further comprising subjecting the denatured sample to a reduction technique followed by an alkylation technique prior to the proteolytic digestion technique.

3. The method of claim 2, wherein the reduction technique comprises subjecting the denatured sample to the reduction technique to produce a reduced sample,
wherein the reduction technique comprises adding an amount of a reducing agent to the denatured sample and incubating for a reducing incubation time.

4. The method of claim 2 or 3, wherein the alkylation technique comprises subjecting the reduced sample to the alkylation technique to produce an alkylated sample,
wherein the alkylation technique comprises adding an amount of an alkylating agent to the reduced sample and incubating substantially in in a low light condition for an alkylation incubation time, and
wherein the alkylated technique comprises quenching the alkylating agent following the alkylation incubation time.

5. A method for proteolytically digesting a biological sample comprising a glycoprotein to produce a proteolytic glycopeptide, the method comprising:
subjecting the biological sample to a thermal denaturation technique to produce a denatured sample,
wherein the thermal denaturation technique comprises subjecting the biological sample to a thermal cycle comprising a thermal treatment of about 60° C. to about 100° C. with a hold time of at least about 1 minute,
wherein a lid temperature during the thermal cycle is at least about 2° C. higher than a temperature of a block temperature during the thermal cycle;
subjecting the denatured sample to a reduction technique to produce a reduced sample, wherein the reduction technique comprises adding an amount of a reducing agent to the denatured sample and incubating for a reducing incubation time;
subjecting the reduced sample to an alkylation technique to produce an alkylated sample, wherein the alkylation technique comprises adding an amount of an alkylating agent to the reduced sample and incubating substantially in a dark or in a low light condition for an alkylation incubation time, and
wherein the alkylated technique comprises quenching the alkylating agent following the alkylation incubation time; and
subjecting the alkylated sample to a proteolytic digestion technique to produce a proteolytically digested sample comprising the proteolytic glycopeptide,
wherein the proteolytic digestion technique comprises adding an amount of one or more proteolytic enzymes and incubating for a digestion incubation time, and
wherein the proteolytic digestion technique comprises quenching the one or more proteolytic enzymes following the digestion incubation time.

6. The method of any one of claims 1-5, wherein the proteolytic glycopeptide comprises a hydrophilic glycan portion.

7. The method of any one of claims 1-5, wherein the proteolytic glycopeptide comprises a hydrophobic glycan portion.

8. The method of any one of claims 1-7, wherein the biological sample is derived from a human.

9. The method of any one of claims 1-8, wherein the biological sample is a blood sample or a derivative thereof.

10. The method of any one of claims 1-9, wherein the biological sample is a plasma sample.

11. The method of any one of claims 1-10, wherein the biological sample is a serum sample.

12. The method of any one of claims 1-11, wherein the biological sample is not subjected to a high-abundant protein depletion technique prior to the thermal denaturation technique.

13. The method of any one of claims 1-12, wherein the thermal cycle comprises a block set temperature of about 60° C. to about 100° C. with a hold time of at least about 1 minute.

14. The method of any one of claims 1-13, wherein the thermal cycle comprises a block ending temperature of about 15° C. to about 40° C.

15. The method of any one of claims 1-14, wherein the thermal cycle comprises a block starting temperature of about 15° C. to about 50° C.

16. The method of any one of claims 1-15, wherein the thermal cycle is performed in a thermal cycler comprising a lid temperature control element.

17. The method of any one of claims 1-16, wherein the thermal cycle comprises a ramp rate between the block set temperature and the block ending temperature of about 1° C./second to about 10° C./second.

18. The method of any one of claims 1-17, wherein the proteolytic digestion technique is performed at a temperature of about 20° C. to about 55° C.

19. The method of any one of claims 1-18, wherein the digestion incubation time is at least about 20 minutes.

20. The method of any one of claims 1-19, wherein the proteolytic digestion technique is performed at a temperature of about 37° C. for at least about 12 hours.

21. The method of any one of claims 18-20, wherein the proteolytic digestion technique is performed using a second thermal cycle,
wherein the lid temperature during the second thermal cycle is at least about 2° C. higher than the temperature of the block temperature during the second thermal cycle.

22. The method of claim 21, wherein the second thermal cycle is performed in a thermal cycler comprising a lid temperature control element.

23. The method of any one of claims 1-22, wherein each of the one or more proteolytic enzymes is selected from the group consisting of trypsin and LysC.

24. The method of claim 23, wherein the trypsin is methylated and/or acetylated.

25. The method of any one of claims 1-23, wherein the amount of the one or more proteolytic enzymes is in a proteolytic enzyme concentration to sample protein weight ratio of about 1:20 to about 1:40.

26. The method of any one of claims 1-25, wherein quenching the one or more proteolytic enzymes is performed using an acid.

27. The method of any one of claims 1-26, wherein the acid is formic acid (FA) or trifluoroacetic acid (TFA), or a mixture thereof.

28. The method of any one of claims 2-27, wherein the reduction technique is performed at a temperature of about 35° C. to about 70° C.

29. The method of any one of claims 2-28, wherein the reduction incubation time is at least about 20 minutes.

30. The method of any one of claims 2-29, wherein the reduction technique is performed at a temperature of about 60° C. for at least about 50 minutes.

31. The method of any one of claims 2-30, wherein the reduction technique is performed using a third thermal cycle,
wherein the lid temperature during the third thermal cycle is at least about 2° C. higher than the temperature of the block temperature during the third thermal cycle.

32. The method of claim 31, wherein the third thermal cycle is performed in a thermal cycler comprising a lid temperature control element.

33. The method of any one of claims 1-22, wherein the reducing agent is dithiothreitol (DTT) or tris(2-carboxyethyl) phosphine (TCEP).

34. The method of claim 33, wherein DTT is added in an amount of about 10 mM to about 100 mM.

35. The method of any one of claims 2-34, wherein the alkylation technique is performed at a temperature of about 20° C. to about 37° C.

36. The method of any one of claims 2-35, wherein the alkylation incubation time is at least about 5 minutes.

37. The method of any one of claims 2-36, wherein the alkylation technique is performed at a temperature of about 20° C. to about 25° C. for at least about 30 minutes.

38. The method of any one of claims 2-37, wherein the alkylating agent is iodoacetamide (IAA).

39. The method of claim 38, wherein IAA is added in an amount of about 10 mM to about 200 mM.

40. The method of any one of claims 4-39, wherein quenching the alkylating agent comprises use of a neutralizing agent.

41. The method of claim 40, wherein the neutralizing agent is DTT.

42. The method of any one of claims 1-4 and 6-41, wherein the proteolytically digested sample is introduced to the LC-MS system without performing an offline desalting technique.

43. The method of any one of claims 1-4 and 6-42, wherein the period of diversion of the LC-MS technique comprises about 1 to about 5 column volumes of the initial eluate that are diverted to waste.

44. The method of any one of claims 1-4, and 6-43, wherein the LC-MS technique is a high pressure LC-MS technique.

45. The method of any one of claims 1-4 and 6-43, wherein the LC-MS technique comprises multiple reaction monitoring.

46. The method of any one of claims 1-4 and 6-45, further comprising adding a standard to the proteolytically digested sample prior to the LC-MS technique.

47. The method of claim 46, wherein the standard is a stable isotope-internal standard (SI-IS) peptide mixture.

48. The method of any one of claims 1-47, wherein the biological sample is admixed with a buffer prior to the thermal denaturation technique.

49. The method of claim 48, wherein the buffer is ammonium bicarbonate.

50. The method of any one of claims 1-49, wherein the proteolytic glycopeptide comprises one or more sialic acid groups.

51. The method of any one of claims 33-50, wherein the proteolytically digested sample introduced to the liquid chromatography (LC) system comprises one or more of the DTT, the IAA, the iodide, and a disulfide bonded 6-membered ring, wherein the disulfide bonded 6-membered ring is a byproduct of DTT.

52. A method for processing a proteolytically digested sample to produce a processed sample suitable for use in a liquid chromatography-mass spectrometry (LC-MS) analysis, wherein the proteolytically digested sample comprises a plurality of proteolytic polypeptides comprising at least one proteolytic glycopeptide,
the method comprising:
performing one or more of the following:
(a) subjecting the proteolytically digested sample to a solid phase extraction column comprising a reversed-phase medium according to one or more conditions to associate at least a portion of the plurality of proteolytic polypeptides with the reversed-phase medium, the one or more conditions comprising:
(i) a polypeptide loading amount of about 50% or less of a binding capacity of the reversed-phase medium,
wherein the binding capacity of the reversed-phase medium is based on an insulin load having 10% or less breakthrough; or
(ii) a polypeptide loading concentration of about 0.6 μg/μL or less; or
(b) subjecting the reversed-phase medium comprising the associated proteolytic polypeptides to a wash buffer at a wash flow rate of about 0.1 column volumes/minute to about 2 column volumes/minute; and
subjecting the reversed-phase medium comprising the associated proteolytic polypeptides to an elution buffer to produce the processed sample.

53. The method of claim 52, wherein the one or more conditions comprises the polypeptide loading amount of about 50% or less of the binding capacity of the reversed-phase medium.

54. The method of claim 52 or 53, wherein the one or more conditions comprises the polypeptide loading concentration of about 0.6 μg/μL or less.

55. The method of any one of claim 52-54, wherein the performing comprises the subjecting the reversed-phase medium comprising the associated proteolytic polypeptides to the wash flow rate of about 0.1 column volumes/minute to about 2 column volumes/minute.

56. The method of any one of claims 52-55, wherein the column comprising the reversed-phase material has a medium volume of about 1 to about 10 μL.

57. The method of any one of claims 52-56, wherein the polypeptide loading amount is about 30 μg to about 200 μg.

58. The method of any one of claims 52-57, wherein the polypeptide loading amount is contained in a solution volume of at least about 100 μL.

59. The method of any one of claims 52-58, wherein the wash flow rate ranges from about 0.5 L/minutes to about 10 μL/minute.

60. The method of any one of claims 52-59, wherein the reversed-phase medium comprises an alkyl-based moiety covalently bound to a solid phase.

61. The method of claim 60, wherein the alkyl-based moiety comprises an octadecyl carbon functional group (C18) covalently bound to the solid phase.

62. The method of claim 60, wherein the alkyl-based moiety comprises an octa carbon functional group (C8) covalently bound to the solid phase.

63. The method of claim 60, wherein the carbon alkyl-based moiety comprises a tetra carbon functional group (C4) covalently bound to the solid phase.

64. The method of any one of claims 60-63, wherein the solid phase comprises a silica material.

65. The method of any one of claims 52-59, wherein the reversed-phase medium comprises a hydrophobic polymer material.

66. The method of claim 65, wherein the hydrophobic polymer material comprises a phenyl moiety.

67. The method of claim 66, wherein the hydrophobic polymer material comprises a reaction product of divinylbenzene.

68. The method of claim 67, wherein the hydrophobic polymer material comprises poly(styrene-co-divinylbenzene).

69. The method of any one of claims 52-68, further comprising subjecting the reversed-phase medium comprising the associated proteolytic polypeptides to a wash buffer prior to subjecting the reversed-phase medium to the elution buffer.

70. The method of any one of claims 52-69, further comprising subjecting the processed sample comprising the elution buffer to a drying technique to produce a dried sample.

71. The method of any one of claims 52-70, further comprising reconstituting the dried sample to produce a reconstituted sample and inputting the reconstituted sample into a LC chromatography system of a LC-MS system to obtain mass spectrometry data.

72. The method of claim 71, further comprising identifying a polypeptide sequence of a glycopeptide from the mass spectrometry data.

73. The method of claim 72, further comprising identifying a glycan attachment site of the glycopeptide from the mass spectrometry data.

74. The method of claim 72 or 73, further comprising identifying a glycan structure of the glycopeptide from the mass spectrometry data.

75. The method of any one of claims 73-74, wherein the at least one glycopeptide comprises a glycan structure comprising one or more sialic acid moieties.

76. The method of any one of claims 52-75, wherein the proteolytically digested sample is obtained from a method for proteolytically digesting a biological sample comprising a glycoprotein.

77. A method for performing a liquid chromatography-mass spectrometry (LC-MS) analysis of a proteolytic glycopeptide derived from a blood sample deposited on a delimited zone of an absorbent or bibulous member
wherein the blood sample comprises a plurality of polypeptides comprising at least one glycoprotein,
the method comprising:
extracting at least a portion of the plurality of polypeptides and one or more extraction internal standards from the absorbent or bibulous member to obtain an extracted sample,
wherein the absorbent or bibulous member comprises the one or more extraction internal standards prior to deposition of the blood sample within the delimited zone, and
wherein at least one of the one or more extraction internal standards comprises a polypeptide standard;
subjecting the extracted sample or a derivative thereof to a proteolytic digestion technique to produce a proteolytically digested sample comprising the proteolytic glycopeptide;
introducing at least a portion of the proteolytically digested sample to a liquid chromatography (LC) system of a LC-MS system; and
performing the LC-MS analysis on at least the proteolytic glycopeptide and the one or more extraction internal standards.

78. The method of claim 77, wherein the performing the LC-MS analysis comprises measuring an abundance signal for the proteolytic glycopeptide and an abundance signal for the one or more extraction internal standards.

79. The method of claim 78, wherein the performing the LC-MS analysis further comprises calculating a concentration of the proteolytic glycopeptide based on a concentration of the one or more extraction internal standards prior to deposition on the absorbent or bibulous member, the abundance signal for the proteolytic glycopeptide, and the abundance signal for the one or more extraction internal standards.

80. The method of claim 77, wherein the absorbent or bibulous member is a dried blood spot card.

81. The method of any one of claims 77-80, further comprising determining an extraction efficiency based on the LC-MS analysis of at least one of the one or more extraction internal standards.

82. The method of any one of claims 77-81, further comprising determining a digestion efficiency based on the LC-MS analysis of at least one of the one or more extraction internal standards.

83. The method of any one of claims 77-82, further comprising assessing a sample migration pattern based on the LC-MS analysis of at least one of the one or more extraction internal standards.

84. The method of any one of claims 77-83, wherein the one or more extraction internal standards comprise a plurality of polypeptide standards, and wherein at least two of the plurality of polypeptide standards have different amino acid lengths.

85. The method of claim 84, wherein the amino acid lengths of the plurality of polypeptide standards of the one or more extraction internal standards range from 4 amino acid to 1500 amino acids.

86. The method of any one of claims 77-85, wherein the at least one polypeptide standard of the one or more extraction internal standards comprises at least one internal enzymatic cleavage site.

87. The method of any one of claims 77-86, wherein the one or more extraction internal standards comprise a plurality of polypeptide standards, wherein at least two of the plurality of polypeptide standards have different net hydrophobicities as based on a computation tool or partition coefficient analysis.

88. The method of claim 87, wherein the plurality of polypeptide standards having different net hydrophobicities comprises a hydrophobicity range of about −0.5 to about 1 according to the Grand average of hydropathicity index (GRAVY).

89. The method of any one of claims 77-88, wherein the at least one polypeptide standard of the one or more extraction internal standards comprises a C-terminal arginine or lysine.

90. The method of any one of claims 77-89, wherein the at least one polypeptide standard of the one or more extraction internal standards comprises an amino acid sequence that does not have homology to a peptide derived from the human proteome.

91. The method of any one of claims 77-90, wherein the at least one polypeptide standard of the one or more extraction internal standards is a synthetic polypeptide.

92. The method of any one of claims 77-91, wherein the at least one polypeptide standard of the one or more extraction internal standards comprises a stable heavy isotope label.

93. The method of any one of claims 77-92, wherein the at least one polypeptide standard of the one or more extraction internal standards comprises a sequence that is non-homologous to an endogenous polypeptide of an individual from which the blood sample originates.

94. The method of any one of claims 77-93, wherein the at least one polypeptide standard of the one or more extraction internal standards is an analog of an endogenous polypeptide of an individual from which the blood sample originates.

95. The method of claim 94, wherein the analog is a stable heavy isotope labeled analog.

96. The method of any one of claims 77-95, wherein the at least one polypeptide standard of the one or more extraction internal standards is a recombinantly expressed polypeptide.

97. The method of any one of claims 77-96, wherein the at least one polypeptide standard of the one or more extraction internal standards is a glycopolypeptide.

98. The method of any one of claims 77-97, wherein the at least one polypeptide standard of the one or more extraction internal standards is a polypeptide that does not substantially interact with hemoglobin.

99. The method of any one of claims 77-98, wherein the at least one polypeptide standard of the one or more extraction internal standards comprises at least a contiguous 4 amino acid sequence from SEQ ID NOS: 14-20.

100. The method of any one of claims 77-99, wherein the at least one polypeptide standard of the one or more extraction internal standards comprises a sequence is selected from the group consisting of SEQ ID NOS: 21-22.

101. The method of any one of claims 77-100, wherein the absorbent or bibulous member comprises a known amount of each of the one or more extraction internal standards.

102. The method of claim 102, wherein the known amount of each of the one or more extraction internal standards is about 0.05 ppm to about 5 ppm.

103. The method of any one of claims 77-103, wherein the one or more extraction internal standards are deposited and dried on the absorbent or bibulous member within an area having a surface area of about 1,000 mm² or less.

104. The method of claim 103, wherein the one or more extraction internal standard are deposited and dried on the absorbent or bibulous member within the delimited zone.

105. The method of any one of claims 77-104, wherein the extracting the at least the portion of the plurality of polypeptides and the one or more extraction internal standards from the absorbent or bibulous member comprises:
separating one or more portions of the absorbent or bibulous member from the absorbent or bibulous member,
wherein the one or more portions of the absorbent or bibulous member comprise at least a portion of the blood sample and the one or more extraction internal standards;
extracting at least the portion of the plurality of polypeptides and the one or more extraction internal standards from the one or more portions of the absorbent or bibulous member into an extraction solution; and
precipitating at least the portion of the plurality of polypeptides and the one or more extraction internal standards to obtain the extracted sample.

106. The method of claim 105, wherein the separating the one or more portions of the absorbent or bibulous member comprises punching the one or more portion of the absorbent or bibulous member using a punching device.

107. The method of claim 105 or 106, wherein each of the one or more portions separated from the absorbent or bibulous member have a surface area of about 2 mm² to about 100 mm².

108. The method of any one of claims 105-107, wherein the precipitating at least the portion of the plurality of polypeptides and the one or more extraction internal standards comprises subjecting the at least the portion of the plurality of polypeptides and the one or more extraction internal standards to ethanol.

109. The method of any one of claims 77-108, further comprising adding a solution to the extracted sample to resolubilize polypeptide content therein prior to subjecting the extracted sample or the derivative thereof to the proteolytic digestion technique.

110. The method of any one of claims 77-109, wherein the proteolytic digestion technique comprises a thermal denaturation technique.

111. The method of claim 110, wherein the proteolytic digestion technique further comprises a reduction technique and an alkylation technique.

112. The method of claim 110 or 111, wherein the proteolytic digestion technique comprises the use of one or more proteases.

113. The method of claim 112, wherein the protease is trypsin.

114. The method of any one of claims 77-108, further comprising adding one or more quantification internal standards after subjecting the extracted sample or the derivative thereof to a proteolytic digestion technique and prior to introducing at least the portion of the proteolytically digested sample to the liquid chromatography LC system of the LC-MS system.

115. The method of any one of claims 77-114, wherein the LC-MS analysis comprises a multiple-reaction-monitoring (MRM) technique targeting the proteolytic glycopeptide and the one or more extraction internal standards.

116. The method of claim 114 or 115, wherein the LC-MS analysis comprises a multiple-reaction-monitoring (MRM) technique targeting the one or more quantification internal standards.

117. The method of any one of claims 77-116, wherein the absorbent or bibulous member comprises a delimited zone having a surface area of about 1,000 mm² or less.

118. The method of any one of claims 77-117, wherein the absorbent or bibulous member comprises a filter paper material.

119. The method of claim 118, wherein the filter paper material comprises a cellulose-based paper.

120. The method of claim 118 or 119, wherein the filter paper material prevents or reduces sample hemolysis.

121. The method of any one of claims 77-120, wherein the absorbent or bibulous member comprises a lateral flow material configured to separate whole blood into a portion of plasma, wherein the whole blood is deposited at the delimited zone and then a liquid portion of the whole blood laterally flows from the delimited zone to a distal zone, wherein the distal zone contains the portion of the plasma.

122. An absorbent or bibulous member comprising one or more extraction internal standard deposited thereon on a delimited zone, wherein the one or more extraction internal standards comprises at least one polypeptide standard, and wherein the absorbent or bibulous member does not comprise a blood sample deposited thereon.

123. The absorbent or bibulous member of claim 122, wherein the absorbent or bibulous member is a blood spot card.

124. A method of classifying a biological sample obtained from a subject with respect to a plurality of states associated with a pelvic cancer, the method comprising receiving peptide structure data corresponding to a set of glycoproteins in the biological sample;
inputting quantification data identified from the peptide structure data for a set of peptide structures into a machine-learning model trained to identify a disease indicator based on the quantification data, wherein the set of peptide structures comprises at least one peptide structure identified from a plurality of peptide structures in Table 9;
identifying, by the machine-learning model, the disease indicator; and
classifying the biological sample with respect to a plurality of states associated with pelvic cancer based upon the identified disease indicator.

125. A method of detecting the presence of one of a plurality of states associated with a pelvic cancer in a subject, the method comprising
receiving peptide structure data corresponding to a set of glycoproteins in a biological sample obtained from a subject, wherein the peptide structure data comprises at least one peptide structure from Table 9;
inputting quantification data identified from the peptide structure data for a set of peptide structures into a machine-learning model trained to identify a disease indicator based on the quantification data; and
detecting the presence of a corresponding state of the plurality of states associated with the pelvic cancer in response to a determination that the identified disease indicator falls within a selected range associated with the corresponding state.

126. The method of claim 124 or 125, wherein the plurality of states comprises at least one of a malignant tumor or a benign tumor.

127. The method of anyone of claims 124-126, wherein the machine-learning model comprises a logistic regression model.

128. The method of any one of claims 124-127, further comprising administering to the subject an effective amount of an agent to treat the pelvic tumor.

129. The method of any one of claims 124-128, wherein the pelvic tumor is ovarian cancer.

130. A method of treating a pelvic tumor in a subject comprising
receiving peptide structure data corresponding to a set of glycoproteins in a biological sample obtained from a subject, wherein the peptide structure data comprises at least one peptide structure from Table 9;
inputting quantification data for the at least one peptide structure into a machine-learning model trained to generate a risk score based on the quantification data;
outputting, by the machine-learning model, the quantification data using the machine learning model to generate a risk score,
administering an effective amount of an agent to treat the pelvic cancer based upon the risk score.

131. A method of determining a diagnosis for a pelvic tumor in a subject comprising receiving peptide structure data corresponding to a set of glycoproteins in a biological sample;
inputting quantification data identified from the peptide structure data for a set of peptide structures into a machine-learning model trained to identify a disease indicator based on the quantification data, wherein the set of peptide structure data comprises at least one peptide structure identified from a plurality of peptide structures in Table 9;
identifying, by the machine-learning model, the disease indicator; and
determining a diagnosis for the pelvic tumor based upon the identified disease indicator.

132. The method of claim 131, wherein the diagnosis is the presence of a malignant tumor or a benign tumor.

133. A method of treating a pelvic tumor in a subject comprising
receiving peptide structure data corresponding to a set of glycoproteins in a biological sample;
inputting quantification data identified from the peptide structure data for a set of peptide structures into a machine-learning model trained to identify a disease indicator based on the quantification data, wherein the peptide structure data comprises at least one peptide structure identified from a plurality of peptide structures in Table 9;
identifying, by the machine-learning model, the disease indicator;
determining a risk score the identified disease indicator; and
administering an effective amount of an agent to treat the pelvic tumor based upon the risk score.

134. A method of treating a pelvic tumor in an individual comprising detecting the presence or amount of at least one peptide structure, wherein the at least one peptide structure comprises at least one peptide structure from Table 9, and administering an effective amount of an agent to treat the pelvic tumor based upon the presence or amount of the peptide structure.

135. A method of diagnosing an individual with a benign or malignant pelvic tumor comprising detecting a presence or amount of at least one peptide structure, wherein the at least one peptide structure comprises at least one peptide structure from Table 9, and diagnosing the individual with a benign or malignant pelvic tumor based upon the presence or amount of the at least one peptide structure.

136. A method of diagnosing an individual with a pelvic tumor comprising
detecting the presence or amount of at least one peptide structure from Table 9;
inputting a quantification of the detected at least one peptide structure into a machine-learning model trained to generate a class label,
determining if the class label is above or below a threshold for a classification;
identifying a diagnostic classification for the individual based on whether the class label is above or below a threshold for the classification; and
diagnosing the individual as having a benign or malignant pelvic tumor on the diagnostic classification.

137. The method of any one of claims 124-133, further comprising detecting the presence or amount of at least one peptide structure from Table 9.

138. The method of any one of claims 134-137, wherein the presence or amount of the at least one peptide structure is detected using mass spectrometry or ELISA.

139. The method of claim 138, wherein the presence or amount of the at least one peptide structure is detected using MRM mass spectrometry.

140. The method of any one of claims 134-139, wherein the amount of at least one peptide structure is none, or below a detection limit.

141. The method of any one of claims 124-140, wherein the at least one peptide structure comprises two or more peptide structures identified in Table 9, three or more peptides structures identified in Table 9, four or more peptide structure identified in Table 9, five or more peptide structures identified in Table 9, six or more peptide structures identified in Table 9, seven or more peptide structures identified in Table 9, or eight or more peptide structure identified in Table 9.

142. The method of any one of claims 124-141 wherein the at least one peptide structure comprises the sequence set forth in SEQ ID NOs: 35-51.

143. The method of any one of claims 124-142, wherein the at least one peptide structure comprises the sequence set forth in SEQ ID NOs: 35-42.

144. The method of any one of claims 124-142, wherein the at least one peptide structure comprises the sequence set forth in SEQ ID NOs: 43-51.

145. The method of any one of claims 124-142, wherein the at least one peptide structure comprises the sequence set forth in SEQ ID NOs: 35-40.

146. The method of any one of claims 124-145, wherein the biological sample is a blood sample, a serum sample, or tumor tissue.

147. The method of claim 146, wherein the biological sample is the blood sample, wherein the blood sample is deposited on a delimited zone of an absorbent or bibulous member comprising a plurality of polypeptides comprising at least one glycoprotein.

148. The method of claim 147 further comprising
extracting at least a portion of the plurality of polypeptides and one or more extraction internal standards from the absorbent or bibulous member to obtain an extracted sample,
wherein the absorbent or bibulous member comprises the one or more extraction internal standards prior to deposition of the blood sample within the delimited zone, and
wherein at least one of the one or more extraction internal standards comprises a polypeptide standard;
subjecting the extracted sample or a derivative thereof to a proteolytic digestion technique to produce a proteolytically digested sample comprising the proteolytic glycopeptide;
introducing at least a portion of the proteolytically digested sample to a liquid chromatography (LC) system of a LC-MS system; and
performing the LC-MS analysis on at least the proteolytic glycopeptide and the one or more extraction internal standards, wherein the at least one proteolytic glycopeptide comprises at least one peptide structure set forth in Table 9.

149. A method for performing a liquid chromatography-mass spectrometry (LC-MS) analysis of a proteolytic glycopeptide derived from a blood sample from an individual deposited on a delimited zone of a blood spot card, the method comprising
obtaining a blood spot card comprising a blood sample from the individual deposited thereon, wherein the blood spot card comprises one or more extraction internal standards deposited and dried prior to deposition of the blood sample on the blood spot card, and
wherein the blood spot card comprising the blood sample contains at least a portion of the blood sample and the one or more extraction internal standards in an overlapping area of the blood spot card;
extracting at least a portion of the plurality of polypeptides and the one or more extraction internal standards from the blood spot card to obtain an extracted sample;
subjecting the extracted sample or a derivative thereof to a proteolytic digestion technique to produce a proteolytically digested sample comprising the proteolytic glycopeptide;
introducing at least a portion of the proteolytically digested sample to a liquid chromatography (LC) system of a LC-MS system; and
performing an LC-MS analysis to quantify one or more biomarkers of ovarian cancer and the one or more extraction internal standards,
wherein the one or more biomarkers comprise a polypeptide comprising a sequence of any of SEQ ID NOs: 35-51, and
wherein at least one of the one or more biomarkers is a glycopeptide.

150. The method of claim 148 or 149, wherein the at least one polypeptide standard of the one or more extraction internal standards comprises at least a contiguous 4 amino acid sequence from SEQ ID NOs: 14-20.

151. The method of any one of claims 148-150, wherein the at least one polypeptide standard of the one or more extraction internal standards comprises a sequence is selected from the group consisting of SEQ ID NOs: 21-22.

152. The method of any one of claims 148-151, wherein the absorbent or bibulous member comprises a known amount of each of the one or more extraction internal standards.

153. The method of any one of claims 148-152, wherein extracting the at least the portion of the plurality of polypeptides and the one or more extraction internal standards from the absorbent or bibulous member comprises:
separating one or more portions of the absorbent or bibulous member from the absorbent or bibulous member,
wherein the one or more portions of the absorbent or bibulous member comprise at least a portion of the blood sample and the one or more extraction internal standards;
extracting at least the portion of the plurality of polypeptides and the one or more extraction internal standards from the one or more portions of the absorbent or bibulous member into an extraction solution; and
precipitating at least the portion of the plurality of polypeptides and the one or more extraction internal standards to obtain the extracted sample.

154. The method of claim 153, wherein the precipitating at least the portion of the plurality of polypeptides and the one or more extraction internal standards comprises subjecting at least the portion of the plurality of polypeptides and the one or more extraction internal standards to ethanol.

155. The method of any one of claims 148-154, further comprising adding a solution to the extracted sample to resolubilize polypeptide content therein prior to subjecting the extracted sample or the derivative thereof to the proteolytic digestion technique.

156. The method of any one of claims 148-155, wherein the proteolytic digestion technique comprises a thermal denaturation technique.

157. The method of claim 156, wherein the proteolytic digestion technique further comprises a reduction technique and an alkylation technique.

158. The method of claim 156 or 157, wherein the proteolytic digestion technique comprises the use of one or more proteases.

159. The method of claim 158, wherein the protease is trypsin.

160. The method of any one of claims 124-159, wherein the absorbent or bibulous member comprises a filter paper material.

161. The method of claim 160, wherein the filter paper material comprises a cellulose-based paper.

162. The method of claim 160 or 161, wherein the filter paper material prevents or reduces sample hemolysis.

163. The method of any one of claims 124-162, wherein the absorbent or bibulous member comprises a lateral flow material configured to separate whole blood into a portion of plasma, wherein the whole blood is deposited at the delimited zone and then a liquid portion of the whole blood laterally flows from the delimited zone to a distal zone, wherein the distal zone contains the portion of the plasma.

164. A method of training a model to diagnose a subject with one of a plurality of states associated with a pelvic tumor, the method comprising
receiving quantification data for a panel of peptide structures for a plurality of subjects
diagnosed with the plurality of states associated with a pelvic tumor
wherein the panel of peptide structures comprises at least one peptide structure set forth in Table 9; and
training a machine-learning model to determine a state of the plurality of states a biological sample from the subject based on the quantification data.

165. The method of claims 124-133 and 164, wherein the quantification data comprises at least one of an abundance, a relative abundance, a normalized abundance, a relative quantity, an adjusted quantity, a normalized quantity, a relative concentration, an adjusted concentration, or a normalized concentration.

166. The method of claim 164 or claim 165, wherein the machine-learning model is trained using random forest or logical progression training methods.

167. The method of any one of claims 164-166, wherein training the machine-learning model to determine the state of the plurality of states comprises training the machine-learning model to generate a class label for the state of the plurality of states.

168. The method of any one of claims 164-167 wherein the machine-learning model comprises a logistic regression model.

169. The method of any one of claims 124-149 and 164-168, wherein at least one of the peptide structures comprises a glycopeptide.

170. A composition comprising one or more peptide structures from Table 9.

171. A composition comprising one or more peptides comprising the sequence set forth in SEQ ID NOs: 35-51.

172. A method for processing a proteolytic digest sample for use in a liquid chromatography-mass spectrometry (LC-MS) analysis,
wherein the proteolytic digest sample comprises a plurality of proteolytically digested peptides comprising at least one proteolytically digested glycopeptide,
the method comprising:
(A) loading a hydrophilic interaction liquid chromatography (HILIC) load derived from the proteolytic digest sample to a solid phase extraction column comprising a HILIC medium according to one or more conditions to associate the at least one proteolytically digested glycopeptide with the HILIC medium, the one or more conditions comprising:
(1) the loading of the HILIC load to the solid phase extraction column is initiated when the HILIC medium is in a dry state;
(2) the HILIC load loaded to the solid phase extraction column has an amount of the plurality of proteolytically digested peptides characterized by one or both of:
(a) a ratio of a weight of the plurality of proteolytically digested peptides over a weight of the HILIC medium in the dry state of at least about 0.06; and/or
(b) a ratio of the weight of the plurality of proteolytically digested peptides relative to a bed volume of the HILIC medium in the dry state of at least about 40 μg/μl; or
(3) the HILIC load loaded to the solid phase extraction column has a concentration of an organic solvent of at least about 70% (v/v); and
(B) subjecting the HILIC medium to an elution liquid to obtain a HILIC eluate comprising the at least one proteolytically digested glycopeptide.

173. The method of claim 172, wherein the one or more loading conditions comprise the loading of the HILIC load to the solid phase extraction column being initiated when the HILIC medium is in the dry state.

174. The method of claim 172 or 173, wherein the HILIC load is characterized by having the ratio of the weight of the plurality of proteolytically digested peptides over the weight of the HILIC medium in the dry state of at least about 0.06.

175. The method of any one of claims 172-174, wherein the HILIC load is characterized by having the ratio of the weight of the plurality of proteolytically digested peptides relative to the bed volume of the HILIC medium in the dry state of at least about 40 μg/μl.

176. The method of any one of claims 172-175, wherein the weight of the HILIC medium in the dry state is about 3 mg or the bed volume of the HILIC medium in the dry state is about 5 UL.

177. The method of claim 176, wherein the HILIC load is characterized by having the ratio of the weight of the plurality of proteolytically digested peptides over the weight of the HILIC medium in the dry state of about 0.1.

178. The method of claim 176 or 177, wherein the HILIC load is characterized by having the ratio of the weight of the plurality of proteolytically digested peptides relative to the bed volume of the HILIC medium in the dry state of about 60 μg/μl.

179. The method of claim 176, wherein the HILIC load is characterized by having the ratio of the weight of the plurality of proteolytically digested peptides over the weight of the HILIC medium in the dry state of about 0.2.

180. The method of claim 176 or 179, wherein the HILIC load is characterized by having the ratio of the weight of the plurality of proteolytically digested peptides relative to the bed volume of the HILIC medium in the dry state of about 120 μg/μl.

181. The method of any one of claims 172-180, wherein the one or more conditions comprise the HILIC load loaded to the solid phase extraction column having the concentration of the organic solvent of at least about 70% (v/v).

182. The method of any one of claims 172-181, wherein the HILIC medium comprises less than about 5% (v/v) of a liquid at the initiation of the loading of the HILIC load to the solid phase extraction column.

183. The method of claim 182, wherein, at the initiation of the loading of the HILIC load to the HILIC medium of the solid phase extraction column, the HILIC medium is not equilibrated with an equilibration liquid.

184. The method of any one of claims 172-183, wherein the HILIC load comprises an amount of the plurality of proteolytically digested peptides of at least about 200 μg.

185. The method of any one of claims 172-184, wherein the concentration of the organic solvent in the HILIC load is at least about 80% (v/v).

186. The method of any one of claims 172-185, wherein the organic solvent comprises an aprotic solvent miscible in water.

187. The method of any one of claims 172-186, wherein the organic solvent is selected from the group consisting of acetonitrile, ethanol, methanol, tetrahydrofuran, and dioxane, or a combination thereof.

188. The method of any one of claims 172-187, further comprising obtaining the HILIC load.

189. The method of claim 188, wherein obtaining the HILIC load comprises reducing a liquid content from the proteolytic digest sample without substantial loss of the plurality of proteolytically digested peptides in the proteolytic digest sample.

190. The method of claim 189, wherein the reducing the liquid content from the proteolytic digested sample comprises performing a peptide concentrating technique with the proteolytically digested sample to obtain a precursor of the HILIC load such that (a) the precursor can be reconstituted with a reconstitution liquid comprising the organic solvent to obtain the HILIC load having a volume of 220 μL or less and a concentration of the organic solvent of at least about 70% (v/v); and (b) the resulting HILIC load comprises an amount of the plurality of proteolytically digested peptides of at least about 200 μg.

191. The method of claim 188, further comprising:
reducing a liquid content from the proteolytic digest sample to form a dried proteolytic digest sample; and
reconstituting the dried proteolytic digest sample with a reconstitution liquid comprising the organic solvent to produce the HILIC load such that (a) the HILIC load has a volume of 220 μL or less and a concentration of the organic solvent of at least about 70% (v/v); and (b) the HILIC load has an amount of the plurality of proteolytic peptides of at least about 200 μg.

192. The method of claim 191, wherein the reconstituting the dried proteolytic digest sample comprises:
mixing the dried proteolytic digest sample with an amount of water to form a water mixture:
sonicating the water mixture with a sonicator;
mixing the water mixture with an amount of trifluoracetic acid (TFA) and acetonitrile (ACN), wherein the amount of TFA and ACN are such that the final concentration of TFA is 1% (v/v) and the final concentration of ACN is 80% (v/v); and
sonicating the water mixture having the amount of TFA and ACN with a sonicator to produce the HILIC load.

193. The method of claim 192, wherein the sonicating the water mixture with the sonicator comprises a water-based dissolution cycle,
wherein the water-based dissolution cycle is repeated about 2 times to about 5 times, and wherein for each of the water-based dissolution cycles, the sonicating the water mixture is performed for about 5 minutes and a water reservoir of the sonicator is configured with ice to cool the water reservoir.

194. The method of claim 192 or 193, wherein the sonicating the water mixture having the amount of TFA and ACN with the sonicator comprises an organic-based dissolution cycle,
wherein the organic-based dissolution cycle is repeated about 2 times to about 3 times, and wherein for each of the organic-based dissolution cycles, the sonicating is performed for about 4 minutes and a water reservoir of the sonicator is configured with ice to cool the water reservoir.

195. The method of any one of claims 189-194, wherein the reducing the liquid content from the proteolytic digest sample comprises removing all or substantially all of the liquid content therefrom.

196. The method of any one of claims 189-194, wherein the peptide concentrating technique comprises a vacuum evaporation technique or a lyophilization technique.

197. The method of any one of claims 172-196, wherein the volume of the HILIC load is 220 μL or less.

198. The method of any one of claims 172-197, wherein the HILIC medium comprises a solid phase or a solid phase comprising a polar functional moiety.

199. The method of claim 150, wherein the solid phase comprises a silica material.

200. The method of claim 198 or 199, wherein the polar functional moiety comprises one or more of an amino group, a cyano group, a carbamoyl group, an aminoalkyl group, alkylamide group, or a combination thereof.

201. The method of any one of claims 172-200, further comprising performing a washing step after loading the HILIC load to the solid phase extraction column and prior to the subjecting the HILIC medium to the elution liquid, wherein the washing step comprises subjecting the HILIC medium to a wash liquid.

202. The method of any one of claims 172-201, further comprising collecting the HILIC eluate, or a fraction thereof, from the solid phase extraction column, wherein the HILIC eluate comprises the at least one proteolytically digested glycopeptide.

203. The method of claim 202, wherein after the collecting the HILIC eluate from the solid phase extraction column, the method further comprises reducing a liquid content of the collected HILIC eluate.

204. The method of any one of claims 172-203, further comprising subjecting the HILIC eluate to a peptide concentrating technique to produce a dried HILIC eluate.

205. The method of claim 204, further comprising reconstituting the dried HILIC eluate to form a sample suitable for introduction to the LC-MS system.

206. The method of claim 205, further comprising injecting the sample suitable for introduction to the LC-MS system into the LC-MS system.

207. The method of any one of claims 172-206, further comprising performing a mass spectrometry technique to obtain mass spectrometry data.

208. The method of claim 207, further comprising identifying a peptide sequence of a glycopeptide from the mass spectrometry data.

209. The method of claim 208, further comprising identifying a glycan attachment site of the glycopeptide from the mass spectrometry data.

210. The method of claim 208 or 209, further comprising identifying a glycan structure of the glycopeptide from the mass spectrometry data.

211. The method of any one of claims 172-210, wherein the at least one glycopeptide comprises a glycan structure comprising one or more sialic acid moieties.

212. The method of any one of claims 172-211, wherein the proteolytic digest sample is obtained from a method for proteolytically digesting a biological sample comprising a glycoprotein.

213. The method of any one of claims 172-212, wherein a glycopeptide concentration for a glycopeptide derived from the proteolytic digest sample is enriched by a factor of 30 or greater with respect to a peptide concentration, wherein the peptide concentration represents an amount of a peptide that is associated with the same protein as the glycopeptide.

214. The method of any one of claims 172-213, further comprising:
measuring a first plurality of peak area values for a first panel of glycopeptides;
measuring a second plurality of peak area values for a second panel of unglycosylated peptides wherein each of the unglycosylated peptides of the second panel corresponds to each of the glycopeptides of the first panel by being attached to a same protein molecule before a proteolytic digestion;
calculating a plurality of ratios by dividing each of the first plurality of peak area values with each of the second plurality of peak area values, respectively; and
determining a median ratio from the plurality of ratios, wherein the median ratio is greater than 30.

215. A method of processing a blood-derived sample obtained from an individual for a glycoproteomic mass spectrometry (MS) technique, the method comprising:
(a) admixing the blood-derived sample with one or more defibrination factors to promote formation of a fibrin clot, the one or more defibrination factors comprises one or more members selected from the group consisting of:
a clotting co-factor;
a clotting enzyme; and
a clotting activator and/or an exogenous surface aggregation agent;
(b) separating the formed fibrin clot from the admixed blood-derived sample to obtain a fibrinogen-depleted sample; and
(c) subjecting the fibrinogen-depleted sample to one or more MS preparation techniques to produce a test sample for the glycoproteomic mass spectrometry technique.

216. The method of claim 215, wherein the one or more defibrination factors comprises the clotting co-factor.

217. The method of claim 216, wherein the clotting co-factor comprises a divalent cation.

218. The method of claim 217, wherein the clotting co-factor comprises the divalent cation, and wherein the divalent cation is Ca²⁺, Mg²⁺, Zn²⁺, or Cu²⁺, or any combination thereof.

219. The method of claim 217 or 218, wherein the divalent cation is Ca²⁺.

220. The method of any one of claims 216-219, wherein the clotting co-factor is calcium chloride, calcium acetate, calcium carbonate, calcium citrate, or calcium gluconate, or any combination thereof.

221. The method of any one of claims 216-220, wherein, following admixing with the blood-derived sample, the clotting co-factor has a concentration of about 5 mM to about 25 mM.

222. The method of any one of claims 215-221, wherein the one or more defibrination factors comprises the clotting enzyme.

223. The method of claim 222, wherein the clotting enzyme is thrombin.

224. The method of claim 222 or 223, wherein, following admixing with the blood-derived sample, the clotting enzyme has a concentration of about 1 unit/mL to 10 units/mL.

225. The method of any one of claim 215-224, wherein the one or more defibrination factors comprises the clotting activator and/or the exogenous surface aggregation agent.

226. The method of claim 225, wherein the clotting activator and/or the exogenous surface aggregation agent is an exogenous surface aggregation agent.

227. The method of claim 226, wherein the exogenous surface aggregation agent comprises Kaolin.

228. The method of claim 225, wherein the clotting activator and/or the exogenous surface aggregation agent is a clotting activator and exogenous surface aggregation agent.

229. The method of claim 228, wherein the clotting activator and exogenous surface aggregation agent comprises a material having pores with an average size of about 2 nm to about 60 nm.

230. The method of claim 228 or 229, wherein the clotting activator and exogenous surface aggregation agent comprises a silica particle.

231. The method of claim 230, wherein the silica particle has a pore size ranging from about 2 to about 60 nm.

232. The method of any one of claims 225-231, wherein the clotting activator and/or the exogenous surface aggregation agent is admixed with the blood-derived sample at an amount of about 50 μg to about 500 μg per 40 μL of the blood-derived sample.

233. The method of any one of claims 215-232, wherein the one or more defibrination factors comprise the clotting co-factor and the clotting enzyme.

234. The method of any one of claims 215-232, wherein the one or more defibrination factors comprise the clotting co-factor and the clotting activator and/or the exogenous surface aggregation agent.

235. The method of any one of claims 215-232, wherein the one or more defibrination factors comprise the clotting enzyme and the clotting activator and/or the exogenous surface aggregation agent.

236. The method of any one of claims 215-232, wherein the one or more defibrination factors comprise the clotting co-factor, the clotting enzyme, and the clotting activator and/or the exogenous surface aggregation agent.

237. The method of any one of claims 215-236, wherein more than one defibrination factor is admixed with the blood-derived sample sequentially.

238. The method of any one of claims 215-236, wherein more than one defibrination factor is admixed with the blood-derived sample simultaneously.

239. The method of any one of claims 215-238, wherein at least one of the one or more defibrination factors is added to a vessel containing the blood-derived sample.

240. The method of any one of claims 215-239, wherein the blood-derived sample is added to a vessel containing at least one of the one or more defibrination factors.

241. The method of any one of claims 215-240, wherein the method further comprises an incubation period following the admixing of the blood-derived sample with one or more defibrination factors.

242. The method of claim 241, wherein the incubation period is about 1 minute to about 30 minutes.

243. The method of any one of claims 215-242, wherein the separating the formed fibrin clot to obtain the fibrinogen-depleted sample comprises subjecting the admixed blood-derived sample with the one or more defibrination factors to a centrifugation technique and/or a filtration technique.

244. The method of any one of claims 215-243, wherein the separating the formed fibrin clot to obtain the fibrinogen-depleted sample comprises subjecting the admixed blood-derived sample with the one or more defibrination factors to a supernatant collection technique.

245. The method of any one of claims 215-244, wherein the fibrinogen-depleted sample is depleted of at least about 80% of the fibrinogen as compared to the blood-derived sample.

246. The method of any one of claims 215-245, wherein the fibrinogen-depleted sample is depleted of at least about 99% of the fibrinogen as compared to the blood-derived sample.

247. The method of any one of claims 215-246, wherein the blood-derived sample is a plasma sample.

248. The method of claim 247, wherein the plasma sample has been treated with an anticoagulant.

249. The method of claim 247 or 248, wherein the plasma sample has been treated with any one or more of the following: a citrate, an ACD (anticoagulant citrate dextrose), Streck, EDTA (ethylenediaminetetraacetic acid), Heparin or Li-Heparin, oxalate fluoride, or a citrate phosphate dextrose adenine (CPDA).

250. The method of any one of claims 215-249, wherein the blood-derived sample is a serum sample.

251. The method of any one of claims 215-250, wherein the one or more MS preparation techniques comprises subjecting the fibrinogen-depleted sample, or a derivative thereof, to a thermal denaturation technique.

252. The method of any one of claims 215-251, wherein the one or more MS preparation techniques comprises subjecting the fibrinogen-depleted sample, or a derivative thereof, to a proteolytic digestion technique.

253. The method of claim 252, wherein the proteolytic digestion technique comprises the use of one or more proteases.

254. The method of claim 253, wherein proteolytic digestion technique comprises the use of trypsin.

255. The method of claim 253 or 254, wherein the one or more proteases are present at a weight ratio of about 1:30 or less, relative to polypeptide content of the fibrinogen-depleted sample, or a derivative thereof.

256. The method of any one of claims 215-255, wherein the one or more MS preparation techniques comprises subjecting the fibrinogen-depleted sample, or a derivative thereof, to a desalting technique.

257. The method of any one of claims 215-256, further comprising performing the glycoproteomic mass spectrometry technique.

258. The method of any one of claims 215-257, wherein the glycoproteomic mass spectrometry technique comprises a liquid chromatography-mass spectrometry (MS) (LC-MS) technique.

259. The method of claim 258, wherein the LC-MS technique comprises a period of diversion of an initial eluate comprising a salt.

260. The method of any one of claims 215-259, wherein the glycoproteomic mass spectrometry technique comprises a multiple-reaction-monitoring (MRM) technique targeting a glycopeptide.

261. A method of preparing a plasma sample obtained from an individual for a glycoproteomic mass spectrometry technique, the method comprising:
(a) admixing the plasma sample with defibrination factors to promote formation of a fibrin clot, the defibrination factors comprising:
a clotting co-factor;
a clotting enzyme; and
a clotting activator and/or an exogenous surface aggregation agent;
(b) separating the formed fibrin clot from the admixed plasma sample to obtain a fibrinogen-depleted sample; and
(c) subjecting the fibrinogen-depleted sample to one or more MS preparation techniques to produce a test sample for the glycoproteomic mass spectrometry technique.

262. The method of claim 261, wherein, after following admixing with the blood-derived sample:
the clotting co-factor comprises Ca²⁺ at a concentration of about 5 mM to about 25 mM;
the clotting enzyme comprises thrombin at a concentration of about 1 unit/mL to 10 units/mL; and
the clotting activator and/or the exogenous surface aggregation agent is in an amount of about 50 μg to about 500 μg per 40 μL of the blood-derived sample.

263. A defibrination composition comprising:
a clotting co-factor;
a clotting enzyme; and
a clotting activator and/or an exogenous surface aggregation agent.

264. A vessel comprising a defibrination composition of claim 263.

265. A method for analyzing a set of peptide structures comprising a linking site, the method comprising:
A) calculating a site occupancy score, for a given peptide structure at the linking site, as a function of an adjusted-raw abundance value for the given peptide structure and a sum of a set of adjusted-raw abundance values of the set of peptide structures; and
B) calculating a monomer weight score as a sum of the site occupancy score and a multiplier, wherein the multiplier is the number of a specific monomer in the set of peptide structures at the linking site.

266. The method of claim 265, further comprising, prior to (A), receiving a set of raw abundance values of the set of peptide structures and normalizing the set of raw abundance values to a corresponding reference run to generate the set of adjusted-raw abundance values.

267. The method of claim 266, further comprising, prior to (B), calculating a peptide structure monomer weight score as a function of the site occupancy score and the number of a specific monomer for the given peptide structure.

268. The method of claim 267, wherein the monomer weight score is a function of the peptide structure monomer weight score and the site occupancy score.

269. The method of any one of claims 265-268, wherein the set of peptide structures is from a biological sample from a subject.

270. The method of claim 269, wherein the biological sample comprises serum or plasma samples.

271. The method of claim 270, wherein the reference run comprises serum or plasma samples.

272. The method of any one of claims 265-271, further comprising:
correlating the monomer weight score with an indication or disease state to determine a hazard ratio for the indication or disease state, wherein the hazard ratio is used to update a risk profile of the subject for the indication or disease state.

273. The method of any one of claims 265-272, further comprising:
generating a diagnosis output for the indication or disease state for the subject, using a predictive model, as a function of the monomer weight score, wherein the diagnosis output is one of a predictive probability or a risk score.

274. The method of claim 273, wherein the predictive model is a logistic regression model, wherein the predictive model generates at least one marker that is correlated with the indication or disease state.

275. The method of any one of claims 265-274, further comprising:
calculating a site occupancy score, for a given peptide structure at the linking site, as the quotient of the adjusted-raw abundance value for the given peptide structure over the sum of the set of adjusted-raw abundance values.

276. The method of any one of claims 265-275, further comprising:
calculating a peptide structure monomer weight score as a product of the site occupancy score and the number of specific monomers for the given peptide structure.

277. The method of claim 276, further comprising:
calculating a monomer weight score for the subject as a sum of peptide structure monomer weight scores for each peptide structure at the linking site.

278. The method of any one of claims 265-277, further comprising:
generating a diagnosis output, based on the monomer weight score, for an indication or disease state, wherein the diagnosis output classifies the biological sample as evidencing a state associated with a disease state progression and/or responsiveness to a specific therapy.

279. The method of any one of claims 265-278, wherein the set of raw abundance values is generated using multiple reaction monitoring mass spectrometry (MRM-MS).

280. The method of any one of claims 265-279, further comprising:
generating a diagnosis output based on the monomer weight score for an indication or disease state, and
generating a treatment output based on at least one of the diagnosis output.

281. The method of claim 280, wherein the treatment output comprises at least one of an identification of a treatment to treat the subject or a treatment plan.

282. The method of claim 281, wherein the treatment comprises at least one of radiation therapy, chemoradiotherapy, surgery, immunotherapy, hormone therapy, or a targeted drug therapy.

283. The method of claim 282, wherein the treatment comprises immunotherapy, wherein the immunotherapy is immune checkpoint blockade therapy.

284. The method of claim 283 wherein the immune checkpoint blockade therapy comprises ipilimumab, nivolumab, and/or pembrolizumab.

285. The method of any one of claims 265-284, further comprising generating a diagnosis output, wherein generating the diagnosis output comprises:
generating a report identifying that the biological sample evidences the indication or disease state.

286. The method of any one of claims 265-285, wherein the specific monomer is selected from the group consisting of hexose, HexNac, fucose, and sialic acid.

287. The method of claim 286, wherein the specific monomer is selected from the group consisting of glucose, mannose, galactose, GlcNAc, GalNAc, fucose, NeuGc, and NeuAc.

288. The method of any one of claims 265-287, further comprising calculating a second monomer weight score as a sum of the site occupancy score and a second multiplier, wherein the second multiplier is the number of a second monomer in the set of peptide structures at the linking site, wherein the second monomer is different from the specific monomer.

289. The method of any one of claims 265-288, further comprising calculating a plurality of additional monomer weight scores as functions of the site occupancy score and a plurality of additional multipliers, wherein the plurality of additional multipliers are the number of a plurality of additional monomers in the set of peptide structures at the linking site.

290. A method of classifying a biological sample with respect to risk of melanoma progression and/or responsiveness to immune checkpoint inhibitor therapy, the method comprising:
A) analyzing one or more monomer weight scores of a set of peptide structures from a biological sample from the subject using a machine learning model to generate a disease indicator; and
B) generating a diagnosis output based on the disease indicator that classifies the biological sample as evidencing a state associated with melanoma progression and/or responsiveness to immune checkpoint inhibitory therapy.

291. The method of claim 290, further comprising:
receiving a set of raw abundance values of the set of peptide structures and normalizing the set of raw abundance values to a corresponding reference run to generate the set of adjusted-raw abundance values.

292. The method claim 291, further comprising:
calculating a site occupancy score, for a given peptide structure at the linking site, as the function of the adjusted-raw abundance value for the given peptide structure and the sum of the set of adjusted-raw abundance values.

293. The method of claim 291 or 292, further comprising:
calculating a site occupancy score, for a given peptide structure at the linking site, as the quotient of the adjusted-raw abundance value for the given peptide structure over the sum of the set of adjusted-raw abundance values.

294. The method of any one of claims 292-293, further comprising:
calculating a peptide structure monomer weight score as a function of the site occupancy score and the number of specific monomers for the given peptide structure.

295. The method of any one of claims 292-294, further comprising:
calculating a peptide structure monomer weight score as a product of the site occupancy score and the number of specific monomers for the given peptide structure.

296. The method of any one of claims 294-295, further comprising:
calculating a monomer weight score of the one or more monomer weight scores as a sum of peptide structure monomer weight scores for each peptide structure at the linking site.

297. The method of any one of claims 290-296, wherein the set of peptide structures comprises post translationally modified (PTM) peptides and/or non-PTM peptides.

298. The method of any one of claims 290-297, wherein the monomer is selected from the group consisting of hexose, HexNac, fucose, and sialic acid.

299. The method of claim 298, wherein the monomer is selected from the group consisting of glucose, mannose, galactose, GlcNAc, GalNAc, fucose, NeuGc, and NeuAc.

300. The method of any one of claims 290-299, wherein the set of peptides structures comprises glycosylated peptides and non-glycosylated peptides.

301. The method of any one of claims 290-300, wherein the biological sample comprises serum or plasma samples.

302. The method of any one of claims 291-301, wherein the reference run comprises serum or plasma samples.

303. The method of any one of claims 290-302, further comprising:
treating the biological sample to form a prepared sample comprising the set of peptide structures, the set of peptide structures comprising a set of post translationally modified (PTM) peptides and/or non-PTM peptides;
detecting a set of product ions associated with each structure of the set of post translationally modified (PTM) peptides and/or non-PTM peptides, and
generating the set of raw abundance values for the set of product ions.

304. The method of any one of claims 290-303, wherein the analyzing further comprises:
correlating the monomer weight score with a melanoma disease state to determine a hazard ratio for the melanoma disease state, wherein the hazard ratio is used to update a risk profile of the subject for the melanoma disease state.

305. The method of any one of claims 290-304, further comprising:
generating a diagnosis output based on the disease indicator that classifies the biological sample as evidencing a state associated with melanoma progression and/or responsiveness to immune checkpoint inhibitory therapy, wherein the diagnosis output is one of a predictive probability or a risk score.

306. The method of any one of claims 290-305, wherein the set of raw abundance values is generated using multiple reaction monitoring mass spectrometry (MRM-MS).

307. The method of any one of claims 290-306, further comprising:
generating a treatment output based on at least one of the diagnosis output.

308. The method of claim 307, wherein the treatment output comprises at least one of an identification of a treatment to treat the subject or a treatment plan.

309. The method of claim 308, wherein the treatment comprises at least one of radiation therapy, chemoradiotherapy, surgery, hormone therapy, or a targeted drug therapy.

310. The method of any one of claims 290-309, wherein generating the diagnosis output comprises:
generating a report identifying that the biological sample evidences the indication or disease state.

311. The method of any one of claims 290-310, wherein the one or more monomer weight scores correspond to at least one site monomer identified in Table 16.

312. The method of any one of claims 290-310, wherein the one or more monomer weight scores correspond to at least one site monomer identified in Table 17.

313. The method of any one of claims 290-310, wherein the one or more monomer weight scores correspond to at least one site monomer identified in Table 18.

314. The method of any one of claims 290-313, further comprising:
training the at least one supervised machine learning model using training data,
wherein the training data comprises a plurality of peptide structure profiles for a plurality of subjects and a plurality of subject diagnoses for the plurality of subjects.

315. The method of claim 314, wherein the plurality of subject diagnoses is selected from the group consisting of a positive diagnosis for any subject of the plurality of subjects determined to have a melanoma disease state, a negative diagnosis for any subject of the plurality of subjects determined not to have a melanoma disease state, a positive diagnosis for any subject of the plurality of subjects determined to be likely to benefit from immune checkpoint inhibitory therapy, and a negative diagnosis for any subject of the plurality of subjects determined to be unlikely to benefit from immune checkpoint inhibitory therapy.

316. The method of claim 315, wherein the plurality of subjects are separated into classes of positive and negative diagnoses using a concordance index as a cutoff between positive and negative diagnoses.

317. The method of any one of claims 314-316, further comprising:
performing a differential expression analysis using the training data to compare a first portion of the plurality of subjects with the positive diagnosis for melanoma disease state or subjects unlikely to benefit from immune checkpoint inhibitory therapy, versus a second portion of the plurality of subjects having the negative diagnosis for melanoma disease state or subjects likely to benefit from immune checkpoint inhibitory therapy; and
identifying a training group of peptide structures based on the differential expression analysis for use as prognostic markers for the melanoma disease state and/or responsiveness to immune checkpoint inhibitory therapy; and
forming the training data based on the training group of peptide structures identified.

318. The method of any one of claims 290-317, wherein the at least one supervised machine learning model comprises a logistic regression model, and wherein the at least one supervised learning model compares the negative diagnosis versus the positive diagnosis, wherein the comparison can be at least one non-melanoma state vs at least one melanoma state, or the comparison can be at least one positive response to immune checkpoint inhibitory therapy vs at least one negative response to immune checkpoint inhibitory therapy.

319. A method of treating melanoma in a subject, the method comprising:
A) analyzing one or more monomer weight scores corresponding to at least one site monomer identified in Table 16 using a machine learning model to generate a diagnosis output that classifies the biological sample as evidencing a state associated with melanoma progression, and
B) administering a therapeutically effective amount of a treatment for melanoma.

320. The method of claim 319, further comprising:
receiving a set of raw abundance values of the set of peptide structures and normalizing the set of raw abundance values to a corresponding reference run to generate the set of adjusted-raw abundance values.

321. The method of claim 320, further comprising:
calculating a site occupancy score, for a given peptide structure at the linking site, as the function of the adjusted-raw abundance value for the given peptide structure and the sum of the set of adjusted-raw abundance values.

322. The method claim 320 or 321, further comprising:
calculating a site occupancy score, for a given peptide structure at the linking site, as the quotient of the adjusted-raw abundance value for the given peptide structure over the sum of the set of adjusted-raw abundance values.

323. The method of any one of claims 321-322, further comprising:
calculating a peptide structure monomer weight score as a function of the site occupancy score and the number of specific monomers for the given peptide structure.

324. The method of any one of claims 321-323, further comprising:
calculating a peptide structure monomer weight score as a product of the site occupancy score and the number of specific monomers for the given peptide structure.

325. The method of claim 323 or 324, further comprising:
calculating the a monomer weight score of the one or more monomer weight scores as a sum of peptide structure monomer weight scores for each peptide structure at the linking site.

326. The method of any one of claims 319-325, wherein the set of peptide structures comprises post translationally modified (PTM) peptides and/or non-PTM peptides.

327. The method of any one of claims 319-326, wherein the monomer is selected from the group consisting of hexose, HexNac, fucose, and sialic acid.

328. The method of claim 327, wherein the monomer is selected from the group consisting of glucose, mannose, galactose, GlcNAc, GalNAc, fucose, NeuGc, and NeuAc.

329. The method of any one of claims 319-328, wherein the set of peptides structures comprises glycosylated peptides and non-glycosylated peptides.

330. The method of any one of claims 319-329, wherein the biological sample comprises serum or plasma samples.

331. The method of any one of claims 320-330, wherein the reference run comprises serum or plasma samples.

332. The method of any one of claims 319-331, further comprising:
treating the biological sample to form a prepared sample comprising the set of peptide structures, the set of peptide structures comprising a set of post translationally modified (PTM) peptides and/or non-PTM peptides;
detecting a set of product ions associated with each structure of the set of post translationally modified (PTM) peptides and/or non-PTM peptides, and
generating the set of raw abundance values for the set of product ions.

333. The method of any one of claims 319-332, wherein the analyzing further comprises:
correlating the one or more monomer weight scores with a melanoma disease state to determine a hazard ratio for the melanoma disease state, wherein the hazard ratio is used to update a risk profile of the subject for the melanoma disease state.

334. The method of any one of claims 319-333, further comprising:
generating a diagnosis output based on a disease indicator that classifies the biological sample as evidencing a state associated with melanoma progression, wherein the diagnosis output is one of a predictive probability or a risk score.

335. The method of any one of claims 319-334, wherein the set of raw abundance values is generated using multiple reaction monitoring mass spectrometry (MRM-MS).

336. The method of any one of claims 319-335, wherein the treatment comprises at least one of radiation therapy, chemoradiotherapy, immunotherapy, surgery, hormone therapy, or a targeted drug therapy.

337. The method of claim 336, wherein the treatment comprises immunotherapy, wherein the immunotherapy is immune checkpoint blockade therapy.

338. The method of claim 337 wherein the immune checkpoint blockade therapy comprises ipilimumab, nivolumab, and/or pembrolizumab.

339. The method of any one of claims 319-338, wherein generating the diagnosis output comprises:
generating a report identifying that the biological sample evidences the indication or disease state.

340. The method of any one of claims 319-339, wherein the one or more monomer weight scores correspond to at least one site monomer identified in Table 17.

341. The method of any one of claims 319-339, wherein the one or more monomer weight scores correspond to at least one site monomer identified in Table 18.

342. The method of any one of claims 319-341, further comprising:
training the at least one supervised machine learning model using training data,
wherein the training data comprises a plurality of peptide structure profiles for a plurality of subjects and a plurality of subject diagnoses for the plurality of subjects.

343. The method of claim 342, wherein the plurality of subject diagnoses is selected from the group consisting of a positive diagnosis for any subject of the plurality of subjects determined to have a melanoma disease state, a negative diagnosis for any subject of the plurality of subjects determined not to have a melanoma disease state, a positive diagnosis for any subject of the plurality of subjects determined to be likely to benefit from immune checkpoint inhibitory therapy, and a negative diagnosis for any subject of the plurality of subjects determined to be unlikely to benefit from immune checkpoint inhibitory therapy.

344. The method of claim 343, wherein the plurality of subjects are separated into classes of positive and negative diagnoses using a concordance index as a cutoff between positive and negative diagnoses.

345. The method of any one of claims 342-344, further comprising:
performing a differential expression analysis using the training data to compare a first portion of the plurality of subjects with the positive diagnosis for melanoma disease state or subjects unlikely to benefit from immune checkpoint inhibitory therapy, versus a second portion of the plurality of subjects having the negative diagnosis for melanoma disease state or subjects likely to benefit from immune checkpoint inhibitory therapy; and
identifying a training group of peptide structures based on the differential expression analysis for use as prognostic markers for the melanoma disease state and/or responsiveness to immune checkpoint inhibitory therapy; and
forming the training data based on the training group of peptide structures identified.

346. The method of any one of claims 319-345, wherein the at least one supervised machine learning model comprises a logistic regression model, and wherein the at least one supervised learning model compares the negative diagnosis versus the positive diagnosis, wherein the comparison can be at least one non-melanoma state vs at least one melanoma state, or the comparison can be at least one positive response to immune checkpoint inhibitory therapy vs at least one negative response to immune checkpoint inhibitory therapy.

347. A system comprising:
one or more data processors; and
a non-transitory computer readable storage medium containing instructions which, when executed on the one or more data processors, cause the one or more data processors to perform part or all of the method of any one of claims 265-346.

348. A computer-program product tangibly embodied in a non-transitory machine-readable storage medium, including instructions configured to cause one or more data processors to perform part or all of the method of any one of claims 265-346.

349. A method of monitoring a subject for a melanoma, the method comprising:
receiving first monomer weight score data for a first biological sample obtained from a subject at a first timepoint;
analyzing the first monomer weight score data using at least one supervised machine learning model to generate a first disease indicator based on at least one site monomer selected from a group of site monomers identified in Table 16, wherein the group of site monomers in Table 16 comprises a group of site monomers having monomer weight scores associated with melanoma;
receiving second monomer weight score data of a second biological sample obtained from the subject at a second timepoint;
analyzing the second monomer weight score data using the at least one supervised machine learning model to generate a second disease indicator based on the at least one site monomer selected from the group of site monomers identified in Table 16; and
generating a diagnosis output based on the first disease indicator and the second disease indicator.

350. The method of claim 349, wherein generating the diagnosis output comprises: comparing the second disease indicator to the first disease indicator.

351. The method of claim 349 or 350, wherein the first disease indicator indicates that the first biological sample evidences a negative diagnosis for melanoma and the second biological sample evidences a positive diagnosis for melanoma.

352. The method of claim 349 or 350, wherein the first disease indicator indicates that the first biological sample evidences a melanoma that is not responsive to immunotherapy and the second biological sample evidences a melanoma that is responsive to immunotherapy.

353. The method of any one of claims 349-352, wherein the at least one supervised machine learning model comprises a logistic regression model, and wherein the at least one supervised learning model compares negative diagnoses versus positive diagnoses, wherein the comparison can be at least one healthy state versus melanoma generally, healthy state versus immunotherapy responsive melanoma, or immunotherapy nonresponsive melanoma versus immunotherapy responsive melanoma.

354. The method of any one of claims 349-353, wherein the at least one site monomer comprises at least one site monomer identified in Table 18.

355. The method of any one of claims 349-353, wherein the at least one site monomer comprises at all site monomers identified in Table 18.

356. A method of treating melanoma in a subject, the method comprising:
determining a monomer weight score for at least one site monomer identified in Table 16 in a biological sample from the subject using a multiple reaction monitoring mass spectrometry (MRM-MS) system;
analyzing the monomer weight score using at least one machine learning model to generate a disease indicator;
generating a diagnosis output based on the disease indicator that classifies the biological sample as evidencing that the patient has melanoma; and
administering to the subject a therapeutically effective amount of a melanoma therapy.

357. The method of claim 356, wherein the melanoma therapy comprises radiation therapy, chemotherapy, chemoradiotherapy, surgery, hormone therapy, immunotherapy, or a targeted drug therapy.

358. The method of claim 357, wherein the melanoma therapy comprises immunotherapy.

359. The method of claim 358, wherein the immunotherapy comprises immune checkpoint blockade therapy.

360. The method of claim 283 wherein the immune checkpoint blockade therapy comprises ipilimumab, nivolumab, and/or pembrolizumab.

361. The method of claim 356, wherein the melanoma therapy does not comprise immunotherapy.

362. The method of any one of claims 356-361, further comprising: preparing the biological sample to form a prepared sample comprising a set of peptide structures; and inputting the prepared sample into the MRM-MS system using a liquid chromatography system.

363. The method of any one of claims 356-362, wherein the at least one site monomer comprises at least one site monomer identified in Table 17.

364. The method of any one of claims 356-362, wherein the at least one site monomer comprises at least one site monomer identified in Table 18.

365. The method of any one of claims 356-362, wherein the at least one site monomer comprises at all site monomers identified in Table 18.

366. A method of treating melanoma in a subject, the method comprising:
determining a monomer weight score for at least one site monomer identified in Table 16 in a biological sample from the subject using a multiple reaction monitoring mass spectrometry (MRM-MS) system;
analyzing the monomer weight score using at least one machine learning model to generate a disease indicator;
generating a diagnosis output based on the disease indicator that classifies the biological sample as evidencing that the melanoma is sensitive to immunotherapy; and
administering to the subject a therapeutically effective amount of immunotherapy.

367. The method of claim 366, wherein the immunotherapy comprises immune checkpoint blockade therapy.

368. The method of claim 367 wherein the immune checkpoint blockade therapy comprises ipilimumab, nivolumab, and/or pembrolizumab.

369. The method of claim 366, 367, or 368, wherein the at least one site monomer comprises at least one site monomer identified in Table 17.

370. The method of claim 366, 367, or 368, wherein the at least one site monomer comprises at least one site monomer identified in Table 18.

371. The method of claim 366, 367, or 368, wherein the at least one site monomer comprises at all site monomers identified in Table 18.

372. A method of identifying a need for one or more medical tests for a subject suspected of being at risk for or having melanoma, the method comprising: subjecting the subject to the one or more medical tests in response to measuring that a biological sample obtained from the subject evidences the subject as having melanoma using part or all of the method of any one of claims 265-346.

373. The method of claim 372, wherein the one or more medical tests comprises colonoscopy, physical exam, CT scan, MRI scan, PET scan, or a combination thereof.

374. A method of designing a treatment for a subject having melanoma, the method comprising:
designing a therapeutic regimen for treating the subject in response to measuring that a biological sample obtained from the subject evidences the subject as having melanoma using part or all of the method of any one of claims 265-346.

375. The method of claim 374, wherein the treatment comprises at least one of radiation therapy, chemotherapy, chemoradiotherapy, immunotherapy, surgery, hormone therapy, or a targeted drug therapy.

376. A method of treating a subject diagnosed with melanoma, the method comprising:
administering to the subject immunotherapy to treat the subject based on measuring that a biological sample obtained from the subject evidences the melanoma as being sensitive to immunotherapy using part or all of the method of any one of claims 265-346.

377. The method of claim 376, wherein the immunotherapy comprises immune checkpoint blockade therapy.

378. A method of classifying a sample from an individual suspected of having, known to have, or at risk for melanoma, comprising the step of determining from the sample a monomer weight score for one or more of the site monomers in Table 16.

379. The method of claim 378, wherein the measuring identifies the individual as not having melanoma.

380. The method of claim 378, wherein the measuring identifies the individual as having melanoma.

381. The method of claim 380, further comprising administering to the individual an effective amount of at least one of radiation therapy, chemotherapy, chemoradiotherapy, immunotherapy, surgery, hormone therapy, or a targeted drug therapy.

382. The method of claim 378, wherein the measuring identifies the individual as having melanoma that is sensitive to immunotherapy.

383. The method of claim 378, wherein the measuring identifies the individual as having melanoma that is not sensitive to immunotherapy.

384. The method of any one of claims 378-383, wherein the sample comprises peripheral blood, plasma, or serum.

385. The method of any one of claims 378-384, wherein the individual is at risk for melanoma.

386. The method of any one of claims 378-385, wherein a monomer weight score is determined for one or more of the site monomers identified in Table 17.

387. The method of any one of claims 378-385, wherein a monomer weight score is determined for one or more of the site monomers identified in Table 18.

388. The method of claim 387, wherein a monomer weight score is determined for all site monomers identified in Table 18.

389. A method of predicting a risk for melanoma in a subject, the method comprising:
determining a monomer weight score for at least one site monomer identified in Table 16 in a biological sample from the subject using a multiple reaction monitoring mass spectrometry (MRM-MS) system;
analyzing the monomer weight score using at least one machine learning model to generate a disease indicator; and
generating a diagnosis output based on the disease indicator that classifies the biological sample as evidencing that the patient has a risk for melanoma.

390. The method of claim 389, wherein the at least one site monomer comprises at least one site monomer identified in Table 17.

391. The method of claim 389, wherein the at least one site monomer comprises at least one site monomer identified in Table 18.

392. The method of claim 389, wherein the at least one site monomer comprises at all site monomers identified in Table 18.

393. A method of predicting immunotherapy sensitivity, the method comprising:
determining a monomer weight score for at least one site monomer identified in Table 16 in a biological sample from the subject using a multiple reaction monitoring mass spectrometry (MRM-MS) system;
analyzing the monomer weight score using at least one machine learning model to generate a disease indicator; and
generating a diagnosis output based on the disease indicator that classifies the biological sample as evidencing that the patient has a risk for melanoma.

394. The method of claim 393, wherein the at least one site monomer comprises at least one site monomer identified in Table 17.

395. The method of claim 393, wherein the at least one site monomer comprises at least one site monomer identified in Table 18.

396. The method of claim 393, wherein the at least one site monomer comprises at all site monomers identified in Table 18.

397. A method of classifying a biological sample with respect to a responsiveness to immune checkpoint inhibitor therapy, the method comprising:
A) analyzing one or more monomer weight scores of a set of peptide structures from a biological sample from the subject using a machine learning model to generate a disease indicator; and
B) generating a diagnosis output based on the disease indicator that classifies the biological sample as evidencing the responsiveness to immune checkpoint inhibitory therapy, wherein the one or more monomer weight scores correspond to at least one site monomer identified in Table 29.

398. A method of classifying a biological sample with respect to a responsiveness to immune checkpoint inhibitor therapy, the method comprising:
A) analyzing one or more monomer weight scores of a set of peptide structures from a biological sample from the subject using a machine learning model to generate a disease indicator;
B) generating a diagnosis output based on the disease indicator that classifies the biological sample as evidencing the responsiveness to immune checkpoint inhibitory therapy, wherein the one or more monomer weight scores correspond to at least one site monomer identified in Table 29; and
C) administering to the subject a therapeutically effective amount of immunotherapy.

399. A method for managing a treatment for a subject diagnosed with a melanoma, the method comprising:
receiving peptide structure data corresponding to a set of glycoproteins in a biological sample obtained from the subject;
computing a treatment score using quantification data identified from the peptide structure data for a set of peptide structures, wherein the set of peptide structures includes at least one peptide structure identified from a plurality of peptide structures listed in Table 23A;
generating a treatment output that indicates a predicted response to the treatment for the subject using the treatment score.

400. The method of claim 399, wherein the at least one peptide structure of Table 23A includes a glycan symbol structure or a glycan composition in accordance with Table 23C.

401. A method for predicting retention times of peptides, by a computing system comprising one or more processors:
accessing a feature set corresponding to a peptide, wherein the feature set represents peptide sequence data of the peptide and corresponding physicochemical features;
sending the feature set as an input into a neural network, the neural network comprising: (1) a plurality of 1DCNN layers, (2) one or more BiLSTM layers, and (3) a multi-head attention layer; and
obtaining, as an output from the neural network, a predicted retention time for the peptide corresponding to an estimated retention time for the peptide in a liquid chromatography mass spectrometry (LC-MS) run.

402. The method of claim 401, wherein the neural network further comprises a flatten and dense layer as a final output layer.

403. The method of claim 401, wherein the feature set for a peptide is generated by:
encoding a peptide sequence of the peptide to generate a matrix representation of the peptide;
compressing the matrix representation to a vector representation; and
concatenating, to the vector representation, one or more corresponding physiochemical features that are determined to be associated with the peptide or peptide sequence.

404. The method of claim 403, wherein generating the feature set further comprises normalizing the concatenated vector representation between 0 and 1.

405. The method of claim 403, wherein the peptide sequence data is encoded using one-hot encoding.

406. The method of claim 405, wherein the matrix representation comprises:
20 columns corresponding to 20 unique amino acids, and
n rows, wherein each row corresponds to a position in a sequence of the corresponding peptide, and wherein n corresponds to a length of the corresponding peptide.

407. The method of claim 403, wherein the peptide sequence data is encoded using BLOSUM 62.

408. The method of claim 407, wherein the encoding generates a matrix comprising:
20 columns corresponding to 20 unique amino acids;
3 columns corresponding to 3 special amino acid characters; and
1 column corresponding to a translation stop.

409. A method of training a neural network for predicting retention times of peptides, by a computing system comprising one or more processors:
accessing a plurality of feature sets corresponding to a plurality of peptides, wherein the feature set represents peptide sequence data of the peptide and corresponding physicochemical features;
creating a training set comprising a subset of feature sets from the plurality of feature sets; and
training a neural network using the training set, the neural network comprising: (1) a plurality of 1DCNN layers, (2) one or more BiLSTM layers, and (3) a multi-head attention layer.

410. The method of claim 409, further comprising:
creating a validation set comprising a subset of feature sets from the plurality of feature sets;
sending the validation set through the neural network; and
evaluating the outputs.

411. The method of claim 410, wherein the training set comprises 80% of the plurality of feature sets and the validation set comprises 30% of the plurality of feature sets.

412. A system for predicting retention times of peptides, the system comprising:
one or more processors; and
a non-transitory memory coupled to the processors comprising instructions executable by the processors, the processors operable when executing the instructions to:
access a feature set corresponding to a peptide, wherein the feature set represents peptide sequence data of the peptide and corresponding physicochemical features;
send the feature set as an input into a neural network, the neural network comprising: (1) a plurality of 1DCNN layers, (2) one or more BiLSTM layers, and (3) a multi-head attention layer. obtain, as an output from the neural network, a predicted retention time for the peptide corresponding to an estimated retention time for the peptide in a liquid chromatography mass spectrometry (LC-MS) run.

413. A system for training a neural network for predicting retention times of peptides, the system comprising:
one or more processors; and
a non-transitory memory coupled to the processors comprising instructions executable by the processors, the processors operable when executing the instructions to:
access a plurality of feature sets corresponding to a plurality of peptides, wherein the feature set represents peptide sequence data of the peptide and corresponding physicochemical features;
create a training set comprising a subset of feature sets from the plurality of feature sets; and
train a neural network using the training set, the neural network comprising: (1) a plurality of 1DCNN layers, (2) one or more BiLSTM layers, and (3) a multi-head attention layer.

414. A computer-readable medium comprising instructions thereon, which when executed by a processor causes the processor to perform the method of any one of claims 401 to 411.
